(19) United States
(12) Patent Application Publication
(10) Pub. No.: US 2018/0349549 A1
(43) Pub. Date: Dec. 6, 2018

(54) Genetic resistance prediction against antimicrobial drugs in microorganism using structural changes in the genome

(71) Applicant: ARES GENETICS GMBH, Vienna (AT)

(72) Inventors: ANDREAS KELLER, Püttlingen (DE); SUSANNE SCHMOLKE, München (DE); CORD FRIEDRICH STÄHLER, Hirschberg an der Bergstraße (DE); CHRISTINA BACKES, Saarbrücken (DE); VALENTINA GALATA, Saarbrücken (DE)

(21) Appl. No.: 15/749,384

(22) PCT Filed: Aug. 5, 2016
(86) PCT No.: PCT/EP2016/068731

§ 371 Date: Jan. 31, 2018

(30) Foreign Application Priority Data
Aug. 6, 2015 (EP) 15180042.2

(51) Int. Cl. G06F 19/16; G06F 19/24; G06F 19/18; G06F 19/22

(52) U.S. Cl. CPC G06F 19/16; G06F 19/24; C12Q 1/04; G06F 19/22; C12Q 2600/156; G06F 19/18

(57) ABSTRACT

Genetic resistance prediction against antimicrobial drugs in microorganism using structural changes in the genome

The invention relates to a method of determining an infection of a patient with at least one microorganism, particularly a bacterial microorganism, potentially resistant to antimicrobial drug treatment, a method of selecting a treatment of a patient suffering from an infection with at least one microorganism, particularly bacterial microorganism, and a method of determining structural changes of the genome of the microorganism, particularly bacterial microorganism, comprising at least one gene, as well as computer program products used in these methods.

The present invention relates to a method of determining an infection of a patient with at least one microorganism, particularly a bacterial microorganism, potentially resistant to antimicrobial drug treatment, a method of selecting a treatment of a patient suffering from an infection with at least one microorganism, particularly bacterial microorganism, and a method of determining structural changes of the genome of the microorganism, particularly bacterial microorganism, comprising at least one gene, as well as computer program products used in these methods.

Antibiotic resistance is a form of drug resistance whereby a sub-population of a microorganism, e.g. a strain of a bacterial species, can survive and multiply despite exposure to an antibiotic drug. It is a serious health concern for the individual patient as well as a major public health issue. Timely treatment of a bacterial infection requires the analysis of clinical isolates obtained from patients with regard to antibiotic resistance, in order to select an efficacious therapy. Generally, for this purpose an association of the identified resistance with a certain microorganism (i.e. ID) is necessary.

Antibacterial drug resistance (ADR) represents a major health burden. According to the World Health Organization's antimicrobial resistance global report on surveillance, ADR leads to 25,000 deaths per year in Europe and 23,000 deaths per year in the US. In Europe, 2.5 million extra hospital days lead to societal cost of 1.5 billion euro. In the US, the direct cost of 2 million illnesses leads to 20 billion dollar direct cost. The overall cost is estimated to be substantially higher, reducing the gross domestic product (GDP) by up to 1.6%.

In general the mechanisms for resistance of bacteria against antimicrobial treatments rely to a very substantial part on the organism's genetics. The respective genes or molecular mechanisms are either encoded in the genome of the bacteria or on plasmids that can be interchanged between different bacteria. The most common resistance mechanisms include:

1) Efflux pumps are high-affinity reverse transport systems located in the membrane that transports the antibiotic out of the cell, e.g. resistance to tetracycline.

2) Specific enzymes modify the antibiotic in a way that it loses its activity. In the case of streptomycin, the antibiotic is chemically modified so that it will no longer bind to the ribosome to block protein synthesis.

3) An enzyme is produced that degrades the antibiotic, thereby inactivating it. For example, the penicillinases are a group of beta-lactamase enzymes that cleave the beta lactam ring of the penicillin molecule.

In addition, some pathogens show natural resistance against drugs. For example, an organism can lack a transport system for an antibiotic or the target of the antibiotic molecule is not present in the organism.

Pathogens that are in principle susceptible to drugs can become resistant by modification of existing genetic material (e.g. spontaneous mutations for antibiotic resistance, happening in a frequency of one in about 100 mio bacteria in an infection) or the acquisition of new genetic material from another source. One example is horizontal gene transfer, a process where genetic material contained in small packets of DNA can be transferred between individual bacteria of the same species or even between different species. Horizontal gene transfer may happen by transduction, transformation or conjugation.

Generally, testing for susceptibility/resistance to antimicrobial agents is performed by culturing organisms in different concentrations of these agents.

In brief, agar plates are inoculated with patient sample (e.g. urine, sputum, blood, stool) overnight. On the next day individual colonies are used for identification of organisms, either by culturing or using mass spectroscopy. Based on the identity of organisms new plates containing increasing concentration of drugs used for the treatment of these organisms are inoculated and grown for additional 12-24 hours. The lowest drug concentration which inhibits growth (minimal inhibitory concentration—MIC) is used to determine susceptibility/resistance for tested drugs. The process takes at least 2 to 3 working days during which the patient is treated empirically. Automated systems exist from several companies, e.g. Biomeriux (Vitek), Beckman Coulter (Microscan). A significant reduction of time-to-result is needed especially in patients with life-threatening disease and to overcome the widespread misuse of antibiotics.

Recent developments include PCR based test kits for fast bacterial identification (e.g. Biomerieux Biofire Tests, Curetis Unyvero Tests). With these test the detection of selected resistance loci is possible for a very limited number of drugs, but no correlation to culture based AST is given. Mass spectroscopy is increasingly used for identification of pathogens in clinical samples (e.g. Bruker Biotyper), and research is ongoing to establish methods for the detection of susceptibility/resistance against antibiotics.

The use of molecular techniques for direct detection of MRSA has become more commonplace especially for screening purposes. Resistance to methicillin is mediated via the mec operon which is part of the staphylococcal cassette chromosome mec (SCCmec). Recently PCR tests were introduced that are based on the detection of the right extremity sequence of the SCCmec in combination with S. aureus specific marker. Initial reports exist that describe culture based susceptibility reports despite detection of the presence of a resistance conferring gene.

For some drugs such it is known that at least two targets are addressed, e.g. in case of Ciprofloxacin (drug bank ID 00537; http://www.drugbank.ca/drugs/DB00537) targets include DNA Topoisomerase IV, DNA Topoisomerase II and DNA Gyrase. It can be expected that this is also the case for other drugs although the respective secondary targets have not been identified yet. In case of a common regulation, both relevant genetic sites would naturally show a co-correlation or redundancy.

It is known that drug resistance can be associated with genetic polymorphisms. This holds for viruses, where resistance testing is established clinical practice (e.g. HIV genotyping). More recently, it has been shown that resistance has also genetic causes in bacteria and even higher organisms, such as humans where tumors resistance against certain cytostatic agents can be linked to genomic mutations.

Wozniak et al. (BMC Genomics 2012, 13(Suppl 7):S23) disclose genetic determinants of drug resistance in Staphylococcus aureus based on genotype and phenotype data. Stoesser et al. disclose prediction of antimicrobial susceptibilities for Escherichia coli and Klebsiella pneumoniae isolates using whole genomic sequence data (J Antimicrob Chemother 2013; 68: 2234-2244).

Chewapreecha et al (Chewapreecha et al (2014) Comprehensive Identification of single nucleotid polymorphisms associated with beta-lactam resistance within pneumococcal mosaic genes. PLoS Genet 10(8): e1004547) used a comparable approach to identify mutations in gram-positive Streptococcus Pneumonia.

In recent studies, genetic tests are taken into account that consider variations in the genome of a microorganism, e.g. a bacterial microorganism. In previous works it could be shown that a faster decision for a treatment could be made using changes in single bases. However, this does not necessarily apply to all antimicrobial drugs, e.g. antibiotics, tested.

The fast and accurate detection of infections with microorganisms, particularly microbial species, e.g. Staphylococcus aureus, and the prediction of response to anti-microbial therapy represent a high unmet clinical need.

This need is addressed by the present invention.

### SUMMARY OF THE INVENTION

The inventors found out that structural variations in the genome that relate to more than one base, particularly at least one gene or more genes in an open reading frame can be related to resistance/susceptibility of microorganisms, particularly bacterial microorganisms, to antimicrobial, e.g. antibiotic, drugs. For example, an efflux pump can be present on a plasmid additionally in a genome. Such efflux pump then can transport a medicine/drug like an antibiotic out of the organism, so that it cannot be effective. Thus, a bacterium having such efflux pump on a plasmid is resistant.

According to a first aspect, the present invention relates to a method of determining structural variations in a genome of a microorganism, particularly a bacterial microorganism, comprising at least a change in the genome comprising more than one base, comprising:

obtaining or providing a first data set of gene sequences of a plurality of clinical isolates of the microorganism, wherein optionally at least a part of the gene sequences of the first data set are assembled; and/or obtaining or providing a first data set of gene sequences of a plurality of clinical isolates of the microorganism and aligning the gene sequences of the first data set to at least one, preferably one, reference sequence;

analyzing the gene sequences of the first data set for structural variations of the genome comprising at least a change in the genome comprising more than one base to obtain a third data set of structural variants;

providing a second data set of antimicrobial drug, e.g. antibiotic, resistance and/or susceptibility of the plurality of clinical isolates of the microorganism;

correlating the third data set with the second data set and statistically analyzing the correlation; and

determining the structural variations in the genome of the microorganism associated with antimicrobial drug, e.g. antibiotic, resistance.

A second aspect of the present invention relates to a diagnostic method of determining an infection of a patient with a microorganism, particularly a bacterial microorganism potentially resistant to antimicrobial drug treatment, comprising the steps of:

a) obtaining or providing a sample containing or suspected of containing a microorganism, particularly a bacterial microorganism, from the patient;

b) determining the presence of at least one structural variation in at least one genetic sequence of the microorganism, particularly bacterial microorganism, as determined by the method of the first aspect, wherein the presence of said at least one structural variation is indicative of an infection with an antimicrobial drug resistant microorganism in said patient.

A third asped of the present invention relates to a method of selecting a treatment of a patient suffering from an infection with a potentially resistant microorganism, particularly bacterial microorganism, comprising the steps of:

a) obtaining or providing a sample containing or suspected of containing a microorganism, particularly a bacterial microorganism, from the patient;

b) determining the presence of at least one structural variation in at least one genetic sequence of the microorganism, particularly bacterial microorganism, as determined by the method of any one of claims 1 to 3, wherein the presence of said at least one structural variation is indicative of a resistance to one or more antimicrobial drugs;

c) identifying said at least one or more antimicrobial drugs; and

d) selecting one or more antimicrobial drugs different from the ones identified in step c) and being suitable for the treatment of the infection with the microorga

A fourth aspect of the present invention relates to a method of determining structural variations of a genome of a microorganism for a clinical isolate of the microorganism, particularly a bacterial microorganism, comprising: obtaining or providing at least one gene sequence of the clinical isolate of the microorganism, particularly the bacterial microorganism; and determining the presence of structural variations in the at least one gene sequence of the clinical isolate of the microorganism, particularly bacterial microorganism, as determined by the method of the first aspect.

In a fifth aspect the present invention relates to one or more computer program products comprising computer executable instructions which, when executed, perform a method according to any one of the first to the fourth aspect of the present invention.

Further aspects and embodiments of the invention are disclosed in the dependent claims and can be taken from the following description, figures and examples, without being limited thereto.

### FIGURES

The enclosed drawings should illustrate embodiments of the present invention and convey a further understanding thereof. In connection with the description they serve as explanation of concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIG. 1 shows schematically a read-out concept for a diagnostic test according to a method of the present invention.

### DETAILED DESCRIPTION OF THE PRESENT INVENTION

Definitions

Unless defined otherwise, technical and scientific terms used herein have the same meaning as commonly understood by one of ordinary skill in the art to which this invention belongs.

An “antimicrobial drug” in the present invention refers to a group of drugs that includes antibiotics, antifungals, antiprotozoals, and antivirals. According to certain embodiments, the antimicrobial drug is an antibiotic.

The term “nucleic acid molecule” refers to a polynucleotide molecule having a defined sequence. It comprises DNA molecules, RNA molecules, nucleotide analog molecules and combinations and derivatives thereof, such as DNA molecules or RNA molecules with incorporated nucleotide analogs or cDNA.

The term “nucleic acid sequence information” relates to an information which can be derived from the sequence of a nucleic acid molecule, such as the sequence itself or a variation in the sequence as compared to a reference sequence.

The term “mutation” relates to a variation in the sequence as compared to a reference sequence. Such a reference sequence can be a sequence determined in a predominant wild type organism or a reference organism, e.g. a defined and known bacterial strain or substrain. A mutation is for example a deletion of one or multiple nucleotides, an insertion of one or multiple nucleotides, or substitution of one or multiple nucleotides, duplication of one or a sequence of multiple nucleotides, translocation of one or a sequence of multiple nucleotides, e.g. also a single nucleotide polymorphism (SNP).

In the context of the present invention a “sample” is a sample which comprises at least one nucleic acid molecule from a bacterial microorganism. Examples for samples are: cells, tissue, body fluids, biopsy specimens, blood, urine, saliva, sputum, plasma, serum, cell culture supernatant, swab sample and others. According to certain embodiments, the sample is a patient sample (clinical isolate).

New and highly efficient methods of sequencing nucleic acids referred to as next generation sequencing have opened the possibility of large scale genomic analysis. The term “next generation sequencing” or “high throughput sequencing” refers to high-throughput sequencing technologies that parallelize the sequencing process, producing thousands or millions of sequences at once. Examples include Massively Parallel Signature Sequencing (MPSS), Polony sequencing, 454 pyrosequencing, Illumina (Solexa) sequencing, SOLiD sequencing, Ion semiconductor sequencing, DNA nanoball sequencing, Helioscope(™) single molecule sequencing, Single Molecule SMRT(™) sequencing, Single Molecule real time (RNAP) sequencing, Nanopore DNA sequencing, Sequencing By Hybridization, Amplicon Sequencing, GnuBio.

Within the present description the term “microorganism” comprises the term microbe. The type of microorganism is not particularly restricted, unless noted otherwise or obvious, and, for example, comprises bacteria, viruses, fungi, microscopic algae and protozoa, as well as combinations thereof. According to certain aspects, it refers to one or more bacterial species, being either Gram-negative or Gram-positive, particularly one or more of Acinetobacter, Escherichia, e.g. E.coli, Enterobacter, Klebsiella, Proteus, Pseudomonas, Salmonella, Serratia, Shigella and/or Staphylococcus species.

A reference to a microorganism or microorganisms in the present description comprises a reference to one microorganism as well a plurality of microorganisms, e.g. two, three, four, five, six or more microorganisms.

A vertebrate within the present invention refers to animals having a vertebrae, which includes mammals—including humans, birds, reptiles, amphibians and fishes. The present invention thus is not only suitable for human medicine, but also for veterinary medicine.

According to certain embodiments, the patient in the present methods is a vertebrate, more preferably a mammal and most preferred a human patient.

Before the invention is described in exemplary detail, it is to be understood that this invention is not limited to the particular component parts of the process steps of the methods described herein as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. For example, the term “a” as used herein can be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

Regarding the dosage of the antimicrobial, e.g. antibiotic, drugs, it is referred to the established principles of pharmacology in human and veterinary medicine. For example, Forth, Henschler, Rummel “Allgemeine and spezielle Pharmakologie and Toxikologie”, 9th edition, 2005 might be used as a guideline. Regarding the formulation of a ready-to-use medicament, reference is made to “Remington, The Science and Practice of Pharmacy”, 22^{nd} edition, 2013.

Assembling of a gene sequence can be carried out by any known method and is not particularly limited.

According to certain embodiments, mutations that were found using alignments can also be compared or matched with alignment-free methods, e.g. for detecting single base exchanges, for example based on contigs that were found by assemblies. For example, reads obtained from sequencing can be assembled to contigs and the contigs can be compared to each other.

A structural variation comprising a change in the genome comprising more than one base refers to a structural variation wherein at least two bases in a gene sequence of a genome of a microorganism that are adjacent are changed, and can refer to e.g. a deletion of multiple (2 or more) nucleotides, an insertion of multiple (2 or more) nucleotides, a substitution of multiple (2 or more) nucleotides, a duplication of a sequence of multiple (2 or more) nucleotides, or a translocation of a sequence of multiple (2 or more) nucleotide. According to certain embodiments, a structural variation can comprise bigger parts sections of the genome, e.g. at least one whole gene in the genome of the microorganism, or even more genes in an open reading frame.

In the present invention, not specific changes of single nucleotides are determined, but a determination is based on bigger parts of the genome, as above.

In the present invention, a reference sequence is not particularly limited, as long as it is useful as a reference for one or more unknown gene sequences in one or more samples, It can for example be one or more reference genomes, a pan genome or one or more centroids. According to certain embodiments, the reference sequences comprise one or more centroids, wherein a centroid is a representative of a gene group/family/cluster of a genome, e.g. of a microorganism. Centroids can be for example extracted from the database MetaRef (http://metaref.org/), which was used in the present examples, with the extraction from the data base being carried out particularly on Nov. 24, 2014. After the extraction the data from the MetaRef database can be updated continually for further experiments. A list of centroids can be extracted for each organism separately or as a whole. In the present examples a list of centroids was extracted for each organism. The centroid information, e.g. for annotation, can be extracted from databases like IMG (http://img.jgi.doe.gov/), as in the present case, or NCBI.

According to a first aspect, the present invention relates to a method of determining structural variations in a genome of a microorganism, particularly a bacterial microorganism, comprising at least a change in the genome comprising more than one base, comprising:

obtaining or providing a first data set of gene sequences of a plurality of clinical isolates of the microorganism, wherein optionally at least a part of the gene sequences of the first data set are assembled; and/or obtaining or providing a first data set of gene sequences of a plurality of clinical isolates of the microorganism and aligning the gene sequences of the first data set to at least one, preferably one, reference sequence;

analyzing the gene sequences of the first data set for structural variations of the genome comprising at least a change in the genome comprising more than one base to obtain a third data set of structural variants;

providing a second data set of antimicrobial drug, e.g. antibiotic, resistance and/or susceptibility of the plurality of clinical isolates of the microorganism;

correlating the third data set with the second data set and statistically analyzing the correlation; and

determining the structural variations in the genome of the microorganism associated with antimicrobial drug, e.g. antibiotic, resistance.

In this method, as well as the other methods of the invention, the first data set of gene sequences of a plurality of clinical isolates can be provided or obtained in any way, preferably non-invasive, and can be e.g. provided from in vitro samples.

According to certain embodiments, the obtaining or providing of gene sequences of a plurality of clinical isolates in this method—as well as the other methods of the invention—can comprise the following:

A sample of a vertebrate, e.g. a human, e.g. is provided or obtained and nucleic acid sequences, e.g. DNA or RNA sequences, are recorded by a known method for recording nucleic acid, which is not particularly limited. For example, nucleic acid can be recorded by a sequencing method, wherein any sequencing method is appropriate, particularly sequencing methods wherein a multitude of sample components, as e.g. in a blood sample, can be analyzed for nucleic acids and/or nucleic acid fragments and/or parts thereof contained therein in a short period of time, including the nucleic acids and/or nucleic acid fragments and/or parts thereof of at least one microorganism of interest, particularly a bacterial microorganism. For example, sequencing can be carried out using polymerase chain reaction (PCR), particularly multiplex PCR, or high throughput sequencing or next generation sequencing, preferably using high-throughput sequencing. For sequencing, preferably an in vitro sample is used.

The data obtained by the sequencing can be in any format, and can then be used to identify the nucleic acids of the microorganism to be identified, by known methods, e.g. fingerprinting methods, comparing genomes and/or aligning to at least one, or more, reference sequences of one or more species of the microorganism of interest, e.g. a reference genome and/or centroids, etc., forming a third data set of, optionally aligned, genes for a microorganism-discarding additional data from other sources, e.g. the vertebrate. For the present method, also the raw data can be used and/or assemblies, at least in part, can be used for forming the third data set. Thus, according to certain embodiments, at least a part of the gene sequences of the first data set can be assembled, wherein assembly can be carried out by any known method and is not particularly limited. In addition, also data from reference sequences, e.g. centroids and/or genomes of known species, e.g. from bacterial species that are already known, e.g. from databases like MetaRef and/or at the NCBI, can be used in the first data set and/or for evaluation of the first data set.

For some organisms, it might be useful in genome-wide association studies to reference the points of interest, e.g. structural variations, to one constant reference for enhanced standardization. In case of the human with a high consistency of the genome and 99% identical sequences among individuals this is easy and represents the standard, as corresponding reference genomes are available in databases.

In case of organisms that trigger infectious diseases (e.g. bacteria and viruses) this is much more difficult, though, and particularly also genetic variations that are not on genes, particularly known genes, can be missed when aligning sequence data to a reference genome. One possibility to overcome this is to fall back on a virtual pan-genome which contains all sequences of a certain genus or to perform reference free variation calling. A further possibility is the analysis of a huge amount of reference sequences, e.g. from MetaRef, and even all available references, which is much more complex. Therein all n references from a database (e.g. RefSeq) are extracted and compared with the newly sequenced bacterial genomes k. After this, matrices (% of mapped reads, % of covered genome) can be applied and the data can be compared to several reference sequences. In such a case, n×k complete alignments are carried out. Having a big number of references, stable results can be obtained.

In the present method, gene sequence of the first data set can also be assembled, at least in part, according to certain embodiments with known methods, e.g. by de-novo assembly or mapping assembly. The sequence assembly is not particularly limited, and any known genome assembler can be used, e.g. based on Sanger, 454, Solexa, Illumina, SOLid technologies, etc., as well as hybrids/mixtures thereof.

According to certain embodiments, the data of nucleic acids of different origin than the microorganism of interest, e.g. a bacterial microorganism, can be removed after the nucleic acids of interest are identified, e.g. by filtering the data out. Such data can e.g. include nucleic acids of a patient, e.g. the vertebrate, e.g. human, and/or other microorganisms, etc. This can be done by e.g. computational subtraction, as developed by Meyerson et al. 2002. For this, also aligning to the genome of the vertebrate, etc., is possible. For aligning, several alignment-tools are available. This way the original data amount from the sample can be drastically reduced.

After such removal of “excess” data, obtaining the third data set can be carried out for the microorganism, e.g. a bacterial microorganism, as described above.

Using these techniques, structural variations in the genome, e.g. in the gene sequences, of the microorganism of interest, e.g. a bacterial microorganism, can be obtained for various species.

When testing these same species for antimicrobial drug, e.g. antibiotic, susceptibility of a number of antimicrobial drugs, e.g. antibiotics, e.g. using standard culturing methods on dishes with antimicrobial drug, e.g. antibiotic, intake, as e.g. described below, the results of these antimicrobial drug, e.g. antibiotic, susceptibility tests can then be cross-referenced/correlated with the structural variations in the genome of the respective microorganism. Using several, e.g. 50 or more than 50, 100 or more than 100, 200 or more than 200, 400 or more than 400, 800 or more than 800, or 900 or more than 900 different isolates of the same or different species of a microorganism, statistical analysis can be carried out on the obtained cross-referenced data between genetic variations and antimicrobial drug, e.g. antibiotic, susceptibility for these microorganisms, using known methods.

Regarding culturing methods, samples of microorganisms can be e.g. cultured overnight. On the next day individual colonies can be used for identification of organisms, either by culturing or using mass spectroscopy. Based on the identity of organisms new plates containing increasing concentration of antibiotics used for the treatment of these organisms are inoculated and grown for additional 12-24 hours. The lowest drug concentration which inhibits growth (minimal inhibitory concentration—MIC) can be used to determine susceptibility/resistance for tested antibiotics.

Also, resistance testing can be carried out by determining e.g. known resistance genes in the different isolates, like in case of methicillin resistant Staphylococcus aureus (MRSA) and methicillin susceptible Staphylococcus aureus (MSSA). For determining resistances, respectively susceptibility, the data from culturing methods and/or from determining known resistance genes, as well as data obtained in different ways, e.g. based on mass spectrometry (possibly also in connection with culturing) can be used.

Correlation of the genetic variations with antimicrobial drug, e.g. antibiotic, resistance can be carried out in a usual way and is not particularly limited. For example, resistances can be correlated to structural variances in the whole genome of the respective microorganism or only parts thereof, for example only coding parts of the genome. In some cases even only genetic variations in genes, e.g. certain genes, or certain mutations in genes can be determined. After correlation, statistical analysis can be carried out.

According to certain embodiments, the data of the first data set can be filtered prior to a possible annotation to a pan-genome and/or reference genome(s) and the correlation with the resistance/susceptibility data.

For example, to reduce the number of similar annotations they can be filtered and aggregated by one or more of the following:
Only annotations for which the considered structural variation lies on a protein can be kept and the further data discarded
Only annotations which do not contain “hypothetical proteins” can be kept
Annotations can be sorted by identification number (ID) and gene product
For a unique pair of IDs and gene products only the first annotation can be kept, e.g in case of multiple genes in a genome

Also, according to certain embodiments, the following structural variations can be excluded:

1. Constant features and phenotypes (same value or only NA) can be removed (e.g. centroids present in all samples or phenotypes with the result “resistant” for all samples)

2. Almost constant features and phenotypes can also removed, e.g. features whose most frequent value was in >=95% of all samples, ignoring NA values, can be removed (e.g. a centroid is present in >=95% of all samples)

Also phenotypes whose most frequent value was in >=90% of all samples, ignoring NA values, can be removed (e.g. >=90% of all samples are resistant)

3. In addition, as is the case in the present examples for the Gram-positive bacteria S. aureus (Gram neg. bacteria in the Examples did not have partially missing data for phenotypes), only drugs with non-missing data for at least 10% of the samples can be kept.

For statistical analysis, as in the examples, e.g. Fisher's exact two-sided test can be applied with subsequent p-value adjustment over all phenotypes together using FDR and p-value threshold of 0.01 (1e-2). Additionally, 10 permutation tests can be performed by permuting each phenotype separately and applying Fisher's exact test to the centroid presence matrix and permuted phenotypes. The results then can be further filtered by centroid annotation, i.e.

1. Centroids without a gene product name can be removed

2. Centroids whose gene product name contains “putative”, “predicted” or “hypothetical” can be removed

3. If there are centroids with same gene product name and gene symbol than only the first one can be kept

4. Centroids without GeneBank accession can be removed

According to certain embodiments, the structural variations can be annotated to a pan-genome of the microorganism and/or annotated to one or more reference sequences, e.g. centroids, of the microorganism. The construction of a pan-genome is not particularly limited and can be done using known methods.

However, other suitable reference genomes (e.g. used in the Examples, but also for other microorganisms) can be found at publicly available data bases like at the NCBI or from MetaRef.

Statistical analysis of the correlation of the gene mutations with antimicrobial drug, e.g. antibiotic, resistance is not particularly limited and can be carried out, depending on e.g. the amount of data, in different ways, for example using analysis of variance (ANOVA), Student's t-test or Fisher's exact test, for example with a sample size n of 50, 100, 200, 300, 400, 500 or 600, and a level of significance (a-error-level) of e.g. 0.05 or smaller, e.g. 0.05, preferably 0.01 or smaller. A statistical value can be obtained for each structural variation and/or each genetic sequence in the genome as well as for all antibiotics tested, a group of antibiotics or a single antibiotic. The obtained p-values can also be adapted for statistical errors, if needed.

For statistically sound results a multitude of individuals should be sampled, with n=50, 100, 200, 300, 400, 500 or 600, and a level of significance (a-error-level) of e.g. 0.05 or smaller, e.g. 0.05, preferably 0.01 or smaller. According to certain embodiments, particularly significant results can be obtained for n=200, 300, 400, 500 or 600.

For statistically sound results a multitude of individuals should be sampled, with n=50 or more, 100 or more, 200 or more, 300 or more, 400 or more, 500 or more or 600 or more, and a level of significance (a-error-level) of e.g. 0.05 or smaller, e.g. 0.05, preferably 0.01 or smaller. According to certain embodiments, particularly significant results can be obtained for n=200 or more, 300 or more, 400 or more, 500 or more or 600 or more.

After the above procedure has been carried out for the individual strains of bacterial species in the Examples, the data disclosed in Tables 1 to 24 were obtained for the statistically best correlations between structural variations and antimicrobial drug, e.g. antibiotic, resistances. In the tables, tables with an odd number, e.g. 1, 3, 5, represent the top 50 results for the statistical analysis as described above, i.e. the structural variations with the best p-value (denoted best_pv in the tables) for at least one antibiotic, whereas the tables with an even number show the top 50 results regarding p-value with at least one drug class ratio of 1.0, so that the structural variation applies to a multitude of drugs and at least a specific drug class, wherein the following applies:

Drug class ratio=number of significant drugs of that class/number of tested drugs of that class For example, if two fluoroquinolones were tested but only one has significant p-value, then the drug class ratio for fluoroquinolones is 0.5.

Genetic variations in the gene sequences given in Tables 1 to 24, with regard to the several reference sequences as above, were proven as valid markers for antimicrobial drug, e.g. antibiotic, resistance. According to certain embodiments, structural variations in at least the gene sequences and/or relating to at least the whole sequences between the start coordinate (denoted “Start.Coord” in the tables) and the end coordinate (denoted “End.Coord” in the tables) of the genes, particularly relating to the whole sequence between the start coordinate and the end coordinate, given for consecutive numbers (“No.”) 1 to 50 with the highest p-values, as above, in Tables 1-24 are obtained by the present method.

When referring to the second data set, wherein the second data set e.g. comprises, respectively is, a set of antimicrobial drug, e.g. antibiotic, resistances of a plurality of clinical isolates, this can, within the scope of the invention, also refer to a self-learning data base that, whenever a new sample is analyzed, can take this sample into the second data set and thus expand its data base. The second data set thus does not have to be static and can be expanded, either by external input or by incorporating new data due to self-learning. This is, however, not restricted to the first aspect of the invention, but applies to other aspects of the invention that refer to a second data set, which does not necessarily have to refer to antimicrobial drug resistance. The same applies, where applicable, to the first data set, e.g. in the first aspect.

According to certain embodiments of the first aspect, the structural variations are detected alignment-free. According to certain embodiments, the structural variations are annotated to a pan-genome of the microorganism and/or annotated to one or more reference sequences.

According to certain embodiments, statistical analysis in the present methods is carried can be carried using Fisher's test with p<10⁻³, preferably p<10⁻⁶, further preferably p<10⁻⁹.

The method of the first aspect of the present invention, as well as related methods, e.g. according to the 2^{nd}, 3^{rd} and 4^{th} aspect, can, according to certain embodiments, comprise correlating different genetic sites to each other. This way even higher statistical significance can be achieved.

According to certain embodiments of the method of the first aspect and related methods-as above, the second data set can be provided by culturing the clinical isolates of the microorganism on agar plates provided with antimicrobial drugs, e.g. antibiotics, at different concentrations, and the second data can be obtained by taking the minimal concentration of the plates that inhibits growth of the respective microorganism.

According to certain embodiments of the method of the first aspect and related methods, the microorganism is a Gram positive bacterial microorganism, particularly a Staphylococcus species, particularly Staphylococcus aureus, and the antimicrobial drug, e.g. antibiotic drug, is selected from the group consisting of β-lactams, β-lactam inhibitors, quinolines and derivatives thereof, e.g. fluoroquinolones, aminoglycosides, glycopeptides, lincosamides, macrolides, nitrofuranes, oxazolidinones polyketides, respectively tetracyclines, and folate synthesis inhibitors, e.g. benzene derived/sulfonamide antibiotics, preferably from the group consisting of Amoxicillin/Clavulanate, Ampicillin, Ampicillin/Sulbactam, Azithromycin, Cefalothin, Cefazolin, Cefepime, Cefotaxime, Cefoxitin, Ceftriaxone, Cefuroxime, Chloramphenicol, Ciprofloxacin, Clindamycin, Daptomycin, Ertapenem, Erythromycin, Fosfomycin, Fusidic acid, Gentamicin, Imipenem, Levofloxacin, Linezolid, Meropenem, Methicillin, Moxifloxacin, Mupirocin, Nitrofurantoin, Norfloxacin, Ofloxacin, Oxacillin, Penicillin G, Piperacillin/Tazobactam, Quinupristin/Dalfopristin, Rifampicin, Teicoplanin, Tetracycline, Tigecycline, Tobramycin, Trimethoprim/Sulfamethoxazole, and Vancomycin, further preferably from the group consisting of Amoxicillin/Clavulanate, Ampicillin, Ampicillin/Sulbactam, Azithromycin, Cefalothin, Cefazolin, Cefepime, Cefotaxime, Cefoxitin, Ceftriaxone, Cefuroxime, Chloramphenicol, Ciprofloxacin, Clindamycin, Daptomycin, Ertapenem, Erythromycin, Fosfomycin, Fusidic acid, Gentamicin, Imipenem, Levofloxacin, Linezolid, Meropenem, Moxifloxacin, Mupirocin, Nitrofurantoin, Norfloxacin, Ofloxacin, Oxacillin, Penicillin G, Piperacillin/Tazobactam, Quinupristin/Dalfopristin, Rifampicin, Teicoplanin, Tetracycline, Tigecycline, Tobramycin, Trimethoprim/Sulfamethoxazole, and Vancomycin.

According to certain embodiments of the method of the first aspect and related methods, the microorganism is a Gram negative bacterial microorganism, particularly one or more of Acinetobacter, Escherichia, e.g. E.coli, Enterobacter, Klebsiella, Proteus, Pseudomonas, Salmonella, Serratia, and/or Shigella species, particularly as denoted in the Examples, and the antimicrobial drug, e.g. antibiotic, is at least one selected from the group of β-lactams, β-lactam inhibitors, quinolines and derivatives thereof, aminoglycosides, polyketides, respectively tetracyclines, and folate synthesis inhibitors, preferably from the group consisting of Amoxicillin/K Clavulanate (AUG), Ampicillin (AM), Aztreonam (AZT), Cefazolin (CFZ), Cefepime (CPM), Cefotaxime (CFT), Ceftazidime (CAZ), Ceftriaxone (CAX), Cefuroxime (CRM), Cephalotin (CF), Ciprofloxacin (CP), Ertapenem (ETP), Gentamicin (GM), Imipenem (IMP), Levofloxacin (LVX), Meropenem (MER), Piperacillin/Tazobactam (P/T), Ampicillin/Sulbactam (A/S), Tetracycline (TE), Tobramycin (TO), and Trimethoprim/Sulfamethoxazole (T/S).

According to certain embodiments, structural variations in at least the gene sequences and/or relating to at least the whole sequences between the start coordinate (denoted “Start.Coord” in the tables) and the end coordinate (denoted “End.Coord” in the tables) of the genes, particularly relating to the whole sequence between the start coordinate and the end coordinate, given for consecutive numbers (“No.”) 1 to 50 with the highest p-values, as above, in Tables 1-24 are obtained by the present method, and the antibiotic is at least one chosen from the class of antibiotics in the column designated “sign_phenos_class”, particularly the one in the column designated “best_pheno_class”, preferably wherein the antibiotic is at least one chosen from the ones in the column designated “sign_phenos”, particularly the one in the column designated “best_pheno”.

A second aspect of the present invention relates to a diagnostic method of determining an infection of a patient with a microorganism, particularly a bacterial microorganism potentially resistant to antimicrobial drug treatment, comprising the steps of:

a) obtaining or providing a sample containing or suspected of containing a microorganism, particularly a bacterial microorganism, from the patient;

b) determining the presence of at least one structural variation in at least one genetic sequence of the microorganism, particularly bacterial microorganism, as determined by the method of the first aspect, wherein the presence of said at least one structural variation is indicative of an infection with an antimicrobial drug resistant microorganism in said patient.

With this method, any mutations in the genome of a microorganism, e.g. the bacterial species given above and below in the Examples, e.g. a clinical isolate with an unknown strain of the microorganism, particularly bacterial microorganism, correlated with antimicrobial drug, e.g. antibiotic, resistance can be determined and a thorough antimicrobial drug, e.g. antibiotic, resistance profile can be established which can add information to a profile which considers only changes in single bases, e.g. single nucleotide polymorphisms (SNPs).

Again, the different steps can herein be carried out as described with regard to the first aspect of the present invention.

According to this aspect, an infection with a microorganism, particularly a bacterial microorganism, in a patient can be determined using sequencing methods, as well as a resistance to antimicrobial drugs, e.g. antibiotics, of the microorganism can be determined in a short amount of time compared to conventional methods.

A third asped of the present invention relates to a method of selecting a treatment of a patient suffering from an infection with a potentially resistant microorganism, particularly bacterial microorganism, comprising the steps of:

a) obtaining or providing a sample containing or suspected of containing a microorganism, particularly a bacterial microorganism, from the patient;

b) determining the presence of at least one structural variation in at least one genetic sequence of the microorganism, particularly bacterial microorganism, as determined by the method of any one of claims 1 to 3, wherein the presence of said at least one structural variation is indicative of a resistance to one or more antimicrobial drugs;

c) identifying said at least one or more antimicrobial drugs; and

d) selecting one or more antimicrobial drugs different from the ones identified in step c) and being suitable for the treatment of the infection with the microorganism, particularly the bacterial microorganism.

This method can be carried out similarly to the second aspect of the invention and enables a fast was to select a suitable treatment with antibiotics for any infection with an unknown microorganism, particularly bacterial microorganism, e.g. Staphylococcus aureus.

In this method, as well as similar ones, no aligning is necessary, as the unknown sample can be directly correlated, after the genome or genome sequences are produced, with the second data set, and thus genetic variations and antimicrobial drug, e.g. antibiotic, resistances can be determined. The first data set can be assembled, for example, using known techniques.

According to certain embodiments, statistical analysis in the present method is carried out using Fisher's test with p<10⁻³, preferably p<10⁻⁶, preferably p<10⁻⁹. Also, according to certain embodiments, the method further comprises correlating different genetic sites to each other.

A fourth aspect of the present invention relates to a method of determining structural variations of a genome of a microorganism for a clinical isolate of the microorganism, particularly a bacterial microorganism, comprising: obtaining or providing at least one gene sequence of the clinical isolate of the microorganism, particularly the bacterial microorganism; and determining the presence of structural variations in the at least one gene sequence of the clinical isolate of the microorganism, particularly bacterial microorganism, as determined by the method of the first aspect.

With this method, antimicrobial drug, e.g. antibiotic, resistances in an unknown isolate of a microorganism, e.g. Staphylococcus aureus, can be determined.

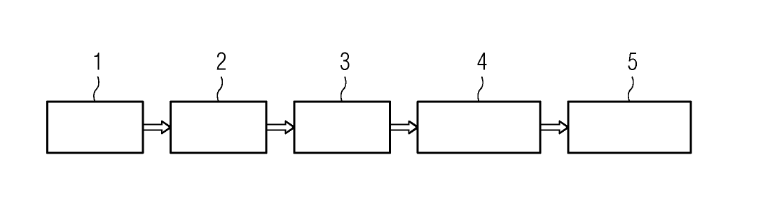

A simple read out concept for a diagnostic test as described in this aspect is shown schematically in FIG. 1.

According to FIG. 1, a sample 1, e.g. blood from a patient, is used for molecular testing 2, e.g. using next generation sequencing (NGS), and then a molecular fingerprint 3 is taken, e.g. in case of NGS a sequence of selected genomic/plasmid regions or the whole genome is assembled. This is then compared to a reference library 4 containing several reference sequences and/or a pan-genome, i.e. selected sequences or the whole sequence are/is compared to one or more reference sequences and/or a pan-genome, and structural variations (,sequence/gene additions/deletions, etc.) are correlated with susceptibility/resistance profiles of reference sequences the reference library. The reference library 4 herein contains many genomes and/or a pan-genome and is different from a reference genome. Then the result 5 is reported which can comprise ID (pathogen identification), i.e. a list of all (pathogenic) species identified in the sample, and AST (antimicrobial susceptibility testing), i.e. a list including a susceptibility/resistance profile for all species listed, based on structural variations.

According to certain embodiments, statistical analysis in the present method is carried out using Fisher's test with p<10⁻³, preferably p<10⁻⁶, preferably p<10⁻⁹. Also, according to certain embodiments, the method further comprises correlating different genetic sites to each other.

Again, in the third and fourth aspect, the different steps can herein be carried out as described with regard to the first aspect of the present invention.

In a fifth aspect the present invention relates to one or more computer program products comprising computer executable instructions which, when executed, perform a method according to any one of the first to the fourth aspect of the present invention.

In certain embodiments the computer program product is one on which program commands or program codes of a computer program for executing said method are stored. According to certain embodiments the computer program product is a storage medium. As noted above, the computer program products of the present invention can be self-learning, e.g. with respect to the first and second data sets.

In order to obtain the best possible information from the highly complex genetic data and develop an optimum model for diagnostic and therapeutical uses as well as the methods of the present invention—which can be applied stably in clinical routine—a thorough in silico analysis can be necessary. The proposed principle is based on a combination of different approaches, e.g. assembly of the gene sequences and/or genome of the microorganisms, at least in part and optionally annotating the sequences to one or more reference sequences and/or a pan-genome, and/or alignment of the sequence data of the clinical isolate to be determined with one or more reference sequences and/or a pan-genome, and correlation of structural variations found in every sample, e.g. from each patient, respectively an unknown clinical isolate, with all references and drugs, e.g. antibiotics, or only one or some of them, and search for structural variations which occur in one or several drug and one or several strains.

Using the above steps a list of structural variations s with regard to one or more reference sequences and/or a pan-genome is generated. These can be stored in databases and statistical models can be derived from the databases. The statistical models can be based on at least one or more structural variations in at least one or more sequences. Statistical models that can be trained can be combined from structural variations and sequences. Examples of algorithms that can produce such models are association Rules, Support Vector Machines, Decision Trees, Decision Forests, Discriminant-Analysis, Cluster-Methods, and many more.

The goal of the training is to allow a reproducible, standardized application during routine procedures.

For this, for example, gene sequences or parts thereof can be sequenced from a patient to be diagnosed. Afterwards, core characteristics can be derived from the sequence data which can be used to predict resistance. These are the points in the database used for the final model, i.e. at least one structural variation, but also combinations of structural variations, etc.

The corresponding characteristics can be used as input for the statistical model and thus enable a prognosis for new patients. Not only the information regarding all resistances of all microorganisms, against all or only some or one drugs, e.g. antibiotics, can be integrated in a computer decision support tool, but also corresponding directives (e.g. EUCAST) so that only treatment proposals are made that are in line with the directives.

An eighth aspect of the present invention relates to the use of the computer program product according to the fifth aspect, e.g. for determining structural variations of a genome of a microorganism for a clinical isolate of the microorganism in the fourth aspect of the invention and/or for use in the diagnostic method of the second method of the invention and/or for selecting a treatment in the third aspect of the present invention and/or in the method of the first aspect of the present invention.

A sixth aspect of the present invention discloses a diagnostic method of determining an infection of a patient with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), potentially resistant to antimicrobial drug, e.g. antibiotic, treatment, comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least two sequences from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 a to 24 a, wherein the presence of said at least two structural variations is indicative of an infection with an antimicrobial drug, e.g. antibiotic, resistant microorganism, preferably bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), in said patient.

As noted above, genetic variations in the gene sequences given in Tables 1 to 24, with regard to the several reference sequences as above, were proven as valid markers for antimicrobial drug, e.g. antibiotic, resistance. According to certain embodiments, structural variations in at least the gene sequences and/or relating to at least the whole sequences between the start coordinate (denoted “Start.Coord” in the tables) and the end coordinate (denoted “End.Coord” in the tables) of the genes, particularly relating to the whole sequence between the start coordinate and the end coordinate, given for consecutive numbers (“No.”) 1 to 50 with the highest p-values, as above, in Tables 1-24 are obtained by the present method.

According to certain embodiments, structural variations in at least the gene sequences and/or relating to at least the whole sequences between the start coordinate (denoted “Start.Coord” in the tables) and the end coordinate (denoted “End.Coord” in the tables) of the genes, particularly relating to the whole sequence between the start coordinate and the end coordinate, given for consecutive numbers (“No.”) 1 to 50 with the highest p-values, as above, in Tables 1-24 are obtained by the present method, and the antibiotic is at least one chosen from the class of antibiotics in the column designated “sign_phenos_class”, particularly the one in the column designated “best_pheno_class”, preferably wherein the antibiotic is at least one chosen from the ones in the column designated “sign_phenos”, particularly the one in the column designated “best_pheno”.

An infection of a patient with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), potentially resistant to antimicrobial drug treatment herein means an infection of a patient with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), wherein it is unclear if the microorganism, preferably bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), is susceptible to treatment with a specific antimicrobial drug or if it is resistant to the antimicrobial drug.

In step b) above, as well as corresponding steps, at least one structural variation in at least two positions is determined, so that in total at least two structural variations are determined, wherein the two structural variations are in different positions, respectively are different sequences.

In this method, as well as the other methods of the invention, the sample can be provided or obtained in any way, preferably non-invasive, and can be e.g. provided as an in vitro sample or prepared as in vitro sample.

According to certain aspects, structural variations in at least two, three, four, five, six, seven, eight, nine or ten positions, respectively sequences, are determined in any of the methods of the present invention, e.g. in at least two positions, respectively sequences, or in at least three positions, respectively sequences. Instead of testing only single positions, respectively sequences, a combination of several variant positions, respectively sequences, can improve the prediction accuracy and further reduce false positive findings that are influenced by other factors. Therefore, it is in particular preferred to determine the presence of structural variations in 2, 3, 4, 5, 6, 7, 8 or 9 (or more) sequences selected from the respective Table 1-24.

According to certain embodiments, the obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient in this method—as well as the other methods of the invention—can comprise the following:

A sample of a vertebrate, e.g. a human, e.g. is provided or obtained and nucleic acid sequences, e.g. DNA or RNA sequences, are recorded by a known method for recording nucleic acid, which is not particularly limited. For example, nucleic acid can be recorded by a sequencing method, wherein any sequencing method is appropriate, particularly sequencing methods wherein a multitude of sample components, as e.g. in a blood sample, can be analyzed for nucleic acids and/or nucleic acid fragments and/or parts thereof contained therein in a short period of time, including the nucleic acids and/or nucleic acid fragments and/or parts thereof of the microorganism. For example, sequencing can be carried out using polymerase chain reaction (PCR), particularly multiplex PCR, or high throughput sequencing or next generation sequencing, preferably using high-throughput sequencing. For sequencing, preferably an in vitro sample is used.

The data obtained by the sequencing can be in any format, and can then be analyzed as described with regard to the first to fourth aspect of the present invention.

In a seventh aspect, the present invention relates to a method of selecting a treatment of a patient suffering from an infection with a potentially resistant microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least two sequences from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 a to 24 a, wherein the presence of said at least two structural variations is indicative of a resistance to one or more antimicrobial, e.g. antibiotic, drugs;

c) identifying said at least one or more antimicrobial, e.g. antibiotic, drugs; and

d) selecting one or more antimicrobial, e.g. antibiotic, drugs different from the ones identified in step c) and being suitable for the treatment of the infection with the microorganism, preferably bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species).

In this method, the steps a) of obtaining or providing a sample and b) of determining the presence of at least one structural variation are as in the method of the sixth aspect.

The identification of the at least one or more antimicrobial, e.g. antibiotic, drug in step c) is then based on the results obtained in step b) and corresponds to the antimicrobial, e.g. antibiotic, drug(s) that correlate(s) with the structural variations. Once these antimicrobial drugs, e.g. antibiotics, are ruled out, the remaining antimicrobial drugs, e.g. antibiotic drugs/antibiotics, can be selected in step d) as being suitable for treatment.

In the description, references to the sixth and seventh aspect also apply to the 9^{th}, 10^{th}, 11^{th} and 12^{th} aspect, referring to the same positions, respectively sequences, unless clear from the context that they don't apply.

According to certain embodiments, the microorganism is a Gram positive bacterial microorganism, particularly a Staphylococcus species, particularly Staphylococcus aureus, and the antimicrobial drug, e.g. antibiotic drug, is selected from the group consisting of β-lactams, β-lactam inhibitors, quinolines and derivatives thereof, e.g. fluoroquinolones, aminoglycosides, glycopeptides, lincosamides, macrolides, nitrofuranes, oxazolidinones polyketides, respectively tetracyclines, and folate synthesis inhibitors, e.g. benzene derived/sulfonamide antibiotics, preferably from the group consisting of Amoxicillin/Clavulanate, Ampicillin, Ampicillin/Sulbactam, Azithromycin, Cefalothin, Cefazolin, Cefepime, Cefotaxime, Cefoxitin, Ceftriaxone, Cefuroxime, Chloramphenicol, Ciprofloxacin, Clindamycin, Daptomycin, Ertapenem, Erythromycin, Fosfomycin, Fusidic acid, Gentamicin, Imipenem, Levofloxacin, Linezolid, Meropenem, Methicillin, Moxifloxacin, Mupirocin, Nitrofurantoin, Norfloxacin, Ofloxacin, Oxacillin, Penicillin G, Piperacillin/Tazobactam, Quinupristin/Dalfopristin, Rifampicin, Teicoplanin, Tetracycline, Tigecycline, Tobramycin, Trimethoprim/Sulfamethoxazole, and Vancomycin, further preferably from the group consisting of Amoxicillin/Clavulanate, Ampicillin, Ampicillin/Sulbactam, Azithromycin, Cefalothin, Cefazolin, Cefepime, Cefotaxime, Cefoxitin, Ceftriaxone, Cefuroxime, Chloramphenicol, Ciprofloxacin, Clindamycin, Daptomycin, Ertapenem, Erythromycin, Fosfomycin, Fusidic acid, Gentamicin, Imipenem, Levofloxacin, Linezolid, Meropenem, Moxifloxacin, Mupirocin, Nitrofurantoin, Norfloxacin, Ofloxacin, Oxacillin, Penicillin G, Piperacillin/Tazobactam, Quinupristin/Dalfopristin, Rifampicin, Teicoplanin, Tetracycline, Tigecycline, Tobramycin, Trimethoprim/Sulfamethoxazole, and Vancomycin.

According to certain embodiments, the microorganism is a Gram negative bacterial microorganism, particularly one or more of Acinetobacter, Escherichia, e.g. E.coli, Enterobacter, Klebsiella, Proteus, Pseudomonas, Salmonella, Serratia, and/or Shigella species, particularly as denoted in the Examples, and the antimicrobial drug, e.g. antibiotic, is at least one selected from the group of β-lactams, β-lactam inhibitors, quinolines and derivatives thereof, aminoglycosides, polyketides, respectively tetracyclines, and folate synthesis inhibitors, preferably from the group consisting of Amoxicillin/K Clavulanate (AUG), Ampicillin (AM), Aztreonam (AZT), Cefazolin (CFZ), Cefepime (CPM), Cefotaxime (CFT), Ceftazidime (CAZ), Ceftriaxone (CAX), Cefuroxime (CRM), Cephalotin (CF), Ciprofloxacin (CP), Ertapenem (ETP), Gentamicin (GM), Imipenem (IMP), Levofloxacin (LVX), Meropenem (MER), Piperacillin/Tazobactam (P/T), Ampicillin/Sulbactam (A/S), Tetracycline (TE), Tobramycin (TO), and Trimethoprim/Sulfamethoxazole (T/S).

In the methods of the invention the resistance of the microorganism, preferably bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), to one or more antimicrobial, e.g. antibiotic, drugs can be determined according to certain embodiments.

According to certain embodiments of the sixth and/or seventh aspect of the invention, the resistance of the microorganism, preferably bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), against 1, 2, 3, 4, 5, 6, 7, 8, 9, 10, 11, 12, 13, 14, 15 or 16, 17, 18, 19, 20 or more antibiotic drugs is determined.

According to certain embodiments of the sixth and/or seventh aspect of the invention, determining the nucleic acid sequence information with the sequences having a structural variation or the presence of a structural variation comprises using a next generation sequencing or high throughput sequencing method. According to preferred embodiments of the sixth and/or seventh aspect of the invention, a partial or entire genome sequence of a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), is determined by using a next generation sequencing or high throughput sequencing method.

A ninth aspect of the present invention is directed to a method of treating a patient suffering from an antimicrobial drug, e.g. antibiotic, resistant infection with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least two sequences from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 to24 a, wherein the presence of said at least two structural variations is indicative of a resistance to one or more antimicrobial, e.g. antibiotic, drugs;

c) identifying said at least one or more antimicrobial, e.g. antibiotic, drugs;

d) selecting one or more antimicrobial, e.g. antibiotic, drugs different from the ones identified in step c) and being suitable for the treatment of the infection with the microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species); and

e) treating the patient with said one or more antimicrobial, e.g. antibiotic, drugs.

Herein, steps a) to d) can be carried out as described with respect to the seventh aspect. Step e) can be sufficiently carried out without being restricted and can be done e.g. non-invasively.

A tenth aspect of the present invention discloses a diagnostic method of determining an infection of a patient with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), potentially resistant to antimicrobial drug, e.g. antibiotic, treatment, comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least one sequence from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 to24 a, wherein the presence of said at least one structural variation is indicative of an infection with an antimicrobial drug, e.g. antibiotic, resistant microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), in said patient.

In an eleventh aspect, the present invention relates to a method of selecting a treatment of a patient suffering from an infection with a potentially resistant microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least one sequence from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 to24 a, wherein the presence of said at least one structural variation is indicative of a resistance to one or more antimicrobial, e.g. antibiotic, drugs;

c) identifying said at least one or more antimicrobial, e.g. antibiotic, drugs; and

d) selecting one or more antimicrobial, e.g. antibiotic, drugs different from the ones identified in step c) and being suitable for the treatment of the infection with the microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species).

Again, in the tenth and the eleventh aspect the steps correspond to those in the sixth or seventh aspect, although only a mutation in at least one gene is determined.

A twelfth aspect of the present invention is directed to a method of treating a patient suffering from an antimicrobial drug, e.g. antibiotic, resistant infection with a microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), comprising the steps of:

a) obtaining or providing a sample containing or suspected of containing at least one microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species), from the patient;

b) determining the presence of at least one structural variation in at least one sequence from the group of sequences annotated with Nos. 1-50 with regard to the reference sequences, particularly centroids (denoted “Scaffold.External.Accession”), with the sequences names, particularly centroid names (denoted “Scaffold.Name”), given in Tables 1 to 24, particularly with the reference genomes and genome names given in Tables 1 to24 a, wherein the presence of said at least one structural variation is indicative of a resistance to one or more antimicrobial, e.g. antibiotic, drugs;

c) identifying said at least one or more antimicrobial, e.g. antibiotic, drugs;

d) selecting one or more antimicrobial, e.g. antibiotic, drugs different from the ones identified in step c) and being suitable for the treatment of the infection with the microorganism, preferably a bacterial microorganism, particularly one as noted above (i.e. one or more of Acinetobacter, Escherichia, e.g. E.coli, Enterobacter, Klebsiella, Proteus, Pseudomonas, Salmonella, Serratia, Shigella and/or Staphylococcus species); and e) treating the patient with said one or more antimicrobial, e.g. antibiotic, drugs.

Also in the twelfth aspect of the invention, steps a) to d) are analogous to the steps in the method of the ninth aspect of the present invention. Step e) can again be sufficiently carried out without being restricted and can be done e.g. non-invasively.

### EXAMPLES

The present invention will now be described in detail with reference to several examples thereof. However, these examples are illustrative and do not limit the scope of the invention.

### Example 1

### Acinetobacter, Particularly Acinetobacter Baumanii

Whole genome sequencing was carried out in addition to classical antimicrobial susceptibility testing of the same isolates for a cohort of 448 specimens. This allowed performing genome wide correlation studies to find genetic variants (e.g. point mutations, small insertions and deletion, larger structural variants, plasmid copy number gains, gene dosage effects) in the genome and plasmids that are significantly correlated to the resistance against one or several drugs. The approach also allows for comparing the relevant sites in the genome to each other.

In the approach the different sources of genetic resistance regarding structural variances as well as the different ways of how bacteria can become resistant were covered. By measuring clinical isolates collected in a broad geographical area and across a broad time span of three decades a complete picture going far beyond the rather artificial step of laboratory generated resistance mechanisms was tried to be generated.

To this end, a set of 21 clinically relevant antimicrobial agents with 5 different modes of action was put together, and the minimally inhibitory concentration (MIC) of the 21 drugs for the Acinetobacter isolates was measured.

The detailed procedure is given in the following:

Bacterial Strains

The inventors selected 448 Acinetobacter strains from the microbiology strain collection at Siemens Healthcare Diagnostics (West Sacramento, Calif.) for susceptibility testing and whole genome sequencing.

Antimicrobial Susceptibility Testing (AST) Panels Frozen reference AST panels were prepared following Clinical Laboratory Standards Institute (CLSI) recommendations. The following antimicrobial agents (with μg/ml concentrations shown in parentheses) were included in the panels:

Amoxicillin/K Clavulanate (0.5/0.25-64/32), Ampicillin (0.25-128), Ampicillin/Sulbactam (0.5/0.25-64/32), Aztreonam (0.25-64), Cefazolin (0.5-32), Cefepime (0.25-64), Cefotaxime (0.25-128), Ceftazidime (0.25-64), Ceftriaxone (0.25-128), Cefuroxime (1-64), Cephalothin (1-64), Ciprofloxacin (0.015-8), Ertepenem (0.12-32), Gentamicin (0.12-32), Imipenem (0.25-32), Levofloxacin (0.25-16), Meropenem (0.12-32), Piperacillin/Tazobactam (0.25/4-256/4), Tetracycline (0.5-64), Tobramycin (0.12-32), and Trimethoprim/Sulfamethoxazole (0.25/4.7-32/608). Prior to use with clinical isolates, AST panels were tested with QC strains. AST panels were considered acceptable for testing with clinical isolates when the QC results met QC ranges described by CLSI16.

Inoculum Preparation

Isolates were cultured on trypticase soy agar with 5% sheep blood (BBL, Cockeysville, Md.) and incubated in ambient air at 35±1° C. for 18-24 h. Isolated colonies (4-5 large colonies or 5-10 small colonies) were transferred to a 3 ml Sterile Inoculum Water (Siemens) and emulsified to a final turbidity of a 0.5 McFarland standard. 2 ml of this suspension was added to 25 ml Inoculum Water with Pluronic-F (Siemens). Using the Inoculator (Siemens) specific for frozen AST panels, 5 μl of the cell suspension was transferred to each well of the AST panel. The inoculated AST panels were incubated in ambient air at 35±1° C. for 16-20 h. Panel results were read visually, and minimal inhibitory concentrations (MIC) were determined.

DNA Extraction

Four streaks of each Gram-negative bacterial isolate cultured on trypticase soy agar containing 5% sheep blood and cell suspensions were made in sterile 1.5 ml collection tubes containing 50 μl Nuclease-Free Water (AM9930, Life Technologies). Bacterial isolate samples were stored at −20° C. until nucleic acid extraction. The Tissue Preparation System (TPS) (096D0382-02_01_B, Siemens) and the VERSANT® Tissue Preparation Reagents (TPR) kit (10632404B, Siemens) were used to extract DNA from these bacterial isolates. Prior to extraction, the bacterial isolates were thawed at room temperature and were pelleted at 2000 G for 5 seconds. The DNA extraction protocol DNAext was used for complete total nucleic acid extraction of 48 isolate samples and eluates, 50 μl each, in 4 hours. The total nucleic acid eluates were then transferred into 96-Well qPCR Detection Plates (401341, Agilent Technologies) for RNase A digestion, DNA quantitation, and plate DNA concentration standardization processes. RNase A (AM2271, Life Technologies) which was diluted in nuclease-free water following manufacturer's instructions was added to 50 μl of the total nucleic acid eluate for a final working concentration of 20 μg/ml. Digestion enzyme and eluate mixture were incubated at 37° C. for 30 minutes using Siemens VERSANT® Amplification and Detection instrument. DNA from the RNase digested eluate was quantitated using the Quant-iT™ PicoGreen dsDNA Assay (P11496, Life Technologies) following the assay kit instruction, and fluorescence was determined on the Siemens VERSANT® Amplification and Detection instrument. Data analysis was performed using Microsoft® Excel 2007. 25 μl of the quantitated DNA eluates were transferred into a new 96-Well PCR plate for plate DNA concentration standardization prior to library preparation. Elution buffer from the TPR kit was used to adjust DNA concentration. The standardized DNA eluate plate was then stored at −80° C. until library preparation.

Next Generation Sequencing

Prior to library preparation, quality control of isolated bacterial DNA was conducted using a Qubit 2.0 Fluorometer (Qubit dsDNA BR Assay Kit, Life Technologies) and an Agilent 2200 TapeStation (Genomic DNA ScreenTape, Agilent Technologies). NGS libraries were prepared in 96 well format using NexteraXT DNA Sample Preparation Kit and NexteraXT Index Kit for 96 Indexes (Illumina) according to the manufacturer's protocol. The resulting sequencing libraries were quantified in a qPCR-based approach using the KAPA SYBR FAST qPCR MasterMix Kit (Peqlab) on a ViiA 7 real time PCR system (Life Technologies). 96 samples were pooled per lane for paired-end sequencing (2×100bp) on Illumina Hiseq2000 or Hiseq2500 sequencers using TruSeq PE Cluster v3 and TruSeq SBS v3 sequncing chemistry (Illumina). Basic sequencing quality parameters were determined using the FastQC quality control tool for high throughput sequence data (Babraham Bioinformatics Institute).

Data Analysis

For each organism in the examples, a list of centroids was extracted from MetaRef (http://metaref.org/). Herein, centroids were representatives of genes, etc. in a group/family/cluster. Data for information/annotation regarding the centroids was extracted from the database IMG (http://img.jgi.doe.gov/).

For Acinetobacter, 1380 centroids of A. baumannii were used as reference sequences, extracted from MetaRef.

The centroid presence in samples was evaluated as follows: Sequences of centroids were aligned against the de novo assemblies using BLASTn (http://blast.ncbi.nlm.nih.gov/Blast.cgi).

The following parameters were used:
BLAST db type: “fasta” and “nucl”
word size: 11 (default)
gapopen: 3 (default 5)
gapextend: 2 (default)
penalty: −2 (default −3)
reward: 1 (default)

A centroids was considered as present if the alignment result contained at least one hit with
min. 80% identity of assembly sequence and centroid sequence
min. 80% of centroid sequence was aligned, computed as

$$centroidcoverage = \frac{centoidalignmentend - centroidalignmentstart + 1}{centroidlength}$$

In the results, a centroid was coded as 0/1 (absent/present)

Association testing and result filtering:

The input data was further filtered as follows to reduce the amount of data:
1. Constant features and phenotypes (same value or only NA) were removed (e.g. centroids present in all samples or phenotypes with “resistant” for all samples)
2. Almost constant features and phenotypes were removed:
   Features whose most frequent value was in >=95% of all samples, ignoring NA values (e.g. a centroid is present in >=95% of all samples), were removed
   Penotypes whose most frequent value was in >=90% of all samples, ignoring NA values (e.g. >=95% of all samples are resistant), were removed 3. Particularly for S. aureus in Example 12, only drugs with non-missing data for at least 10% of the samples were kept

For statistical analysis, Fisher's exact two-sided test was applied with subsequent p-value adjustment over all phenotypes together using FDR and p-value threshold of 0.01 (1e-2). Additionally, 10 permutation tests were performed by permuting each phenotype separately and applying Fisher's exact test to the centroid presence matrix and permuted phenotypes. The results were further filtered by centroid annotation, i.e.
1. Centroids without a gene product name were removed
2. Centroids whose gene product name contains “putative”, “predicted” or “hypothetical” were removed
3. If there are centroids with same gene product name and gene symbol than only the first one was kept
4. Centroids without GeneBank accession were removed

After the above procedure has been carried out, the data disclosed in Tables 1 and 2 were obtained for the statistically best correlations between structural variations and antimicrobial drug, e.g. antibiotic, resistances. Table 1 (as well as the odd Tables 3, 5, 7, 9, 11, 13, 15, 17, 19, 21 and 23 in Examples 2 to 12) represents the top 50 results for structural variations with the best p-value (denoted best_pv in the tables) for at least one antibiotic, whereas Table 2 (as well as the even Tables 4, 6, 8, 10, 12, 14, 16, 18, 20, 22 and 24 in Examples 2 to 12) shows the top 50 results regarding p-value with at least one drug class ratio of 1.0, so that the structural variation applies to a multitude of drugs and at least a specific drug class, wherein the following applies:

Drug class ratio=number of significant drugs of that class/number of tested drugs of that class

For example, if two fluoroquinolones were tested but only one has significant p-value, then the drug class ratio for fluoroquinolones is 0.5.

In Tables 1 and 2, as well as also the following Tables 3 to 24 in Examples 2 to 12, the columns are designated as follows:
No.: Consecutive number from 1-50, corresponding to the best p-values
best_pheno: Phenotype (drug) with smallest adj. p-value
best_pheno_class: drug class of best drug
best_pv: adj. p-value of best phenotype calculated using Fisher's exact test and adjusted by FDR (Benjamini Hochberg) method (Benjamini Hochberg, 1995)
sign_phenos: names of all phenotypes with significant adj. p-value separated by “i”
sign_phenos_class: drug classes of all significant drugs
num_<drug class>: number of significant drugs of that drug class
<phenotype>_pv_adj: adjusted p-value of the phenotype calculated using Fisher's exact test and adjusted by FDR (Benjamini Hochberg) method (Benjamini Hochberg, 1995)
Centroid (gene) annotation:
   Locus.Tag: Locus of the gene in the genome
   Gene.Symbol: Gene symbol
   GeneBank.Accession: GeneBank ID for the gene
   Chromosom: If gene lies on a plasmids, this column contains plasmid name
   Start.Coord/End.Coord: Start and end coordinates of the gene
   Strand: “+” for forward and “−” for reverse strand
   Scaffold.ID: (IMG) ID of the scaffold containing the gene
   Scaffold.External.Accession: Non-IMG ID, e.g. as annotated at the NCBI, RefSeq ID
   Scaffold.Name: Genome name and scaffold external accession ID

TABLE 1a
Data for Acinetobacter, particularly Acinetobacter baumanii

| | Scaffold.External | | |
|---|---|---|---|
| No. | Accession | Scaffold.Name | best_pv |
| 1 | CP001921 | Acinetobacter baumannii 1656-2: CP001921 | 7.16783907123597e−62 |
| 2 | NZ_ACYR01000063 | Acinetobacter sp. 6013113 NZ_ACYR01000063: NZ_ACYR01000063 | 1.39202289199418e−61 |
| 3 | NC_009085 | Acinetobacter baumannii ATCC 17978: NC_009085 | 1.43720401820086e−56 |
| 4 | NC_011595 | Acinetobacter baumannii AB307-0294: NC_011595 | 5.41948460225177e−51 |
| 5 | NC_011595 | Acinetobacter baumannii AB307-0294: NC_011595 | 7.00413054521082e−47 |
| 6 | NC_011586 | Acinetobacter baumannii AB0057: NC_011586 | 2.06435524702153e−46 |
| 7 | NC_011586 | Acinetobacter baumannii AB0057: NC_011586 | 8.98962816077673e−46 |
| 8 | NC_011595 | Acinetobacter baumannii AB307-0294: NC_011595 | 2.76883973579195e−44 |
| 9 | NC_011586 | Acinetobacter baumannii AB0057: NC_011586 | 6.86281420221907e−43 |
| 10 | NZ_ABXK01000013 | Acinetobacter baumannii AB900: NZ_ABXK01000013 | 5.20862955909241e−42 |
| 11 | NZ_GG704495 | Acinetobacter sp. RUH2624 genomic scaffold supercont1.1: NZ_GG704495 | 9.06181183555984e−42 |
| 12 | NZ_GG704515 | Acinetobacter sp. RUH2624 genomic scaffold supercont1.21: NZ_GG704515 | 9.06181183555984e−42 |
| 13 | NZ_GG704515 | Acinetobacter sp. RUH2624 genomic scaffold supercont1.21: NZ_GG704515 | 9.06181183555984e−42 |
| 14 | NC_014259 | Acinetobacter sp. DR1 chromosome: NC_014259 | 9.06181183555984e−42 |
| 15 | NZ_GG704949 | Acinetobacter calcoaceticus RUH2202 genomic scaffold supercont1.1: | 9.06181183555984e−42 |
| | | NZ_GG704949 | |
| 16 | NZ_GG704515 | Acinetobacter sp. RUH2624 genomic scaffold supercont1.21: NZ_GG704515 | 9.06181183555984e−42 |
| 17 | CP002177 | Acinetobacter calcoaceticus PHEA-2: CP002177 | 9.06181183555984e−42 |
| 18 | NZ_GG704515 | Acinetobacter sp. RUH2624 genomic scaffold supercont1.21: NZ_GG704515 | 3.84309843417235e−41 |
| 19 | NZ_GG704495 | Acinetobacter sp. RUH2624 genomic scaffold supercont1.1: NZ_GG704495 | 3.89818602393582e−41 |
| 20 | NC_005966 | Acinetobacter sp. ADP1: NC_005966 | 1.28694655921313e−40 |
| 21 | NZ_GG704515 | Acinetobacter sp. RUH2624 genomic scaffold supercont1.21: NZ_GG704515 | 1.49093940399756e−40 |
| 22 | NC_011595 | Acinetobacter baumannii A3307-0294: NC_011595 | 7.70079013246134e−40 |
| 23 | CP002522 | Acinetobacter baumannii TCDC-AB0715: CP002522 | 9.96749396638303e−40 |
| 24 | NC_011595 | Acinetobacter baumannii AB307-0294: NC_011595 | 1.66049576626689e−39 |
| 25 | NC_011586 | Acinetobacter baumannii AB0057: NC_011586 | 2.08472354496451e−39 |
| 26 | NC_014259 | Acinetobacter sp. DR1 chromosome: NC_014259 | 6.40954171412503e−39 |
| 27 | NZ_GG705093 | Acinetobacter lwoffii SH145 genomic scaffold supercont1.39: NZ_GG705093 | 1.2155660857651e−38 |
| 28 | NZ_ACYQ01000024 | Acinetobacter sp. 6013150 NZ_ACYQ01000024: NZ_ACYQ01000024 | 2.19910980623625e−38 |
| 29 | NZ_GG704532 | Acinetobacter sp. RUH2624 genomic scaffold supercont1.38: NZ_GG704532 | 5.31361687748575e−38 |
| 30 | NZ_ACYS01000159 | Acinetobacter sp. 6014059 NZ_ACYS01000159: NZ_ACYS01000159 | 6.25755791192393e−38 |
| 31 | NC_010611 | Acinetobacter baumannii ACICU: NC_010611 | 1.6998208344511e−37 |
| 32 | NZ_GG753601 | Acinetobacter sp. SH024 genomic scaffold supercont1.2: NZ_GG753601 | 2.25174704599278e−37 |
| 33 | CP002522 | Acinetobacter baumannii TCDC-AB0715: CP002522 | 2.43979456355917e−37 |
| 34 | NZ_GG704979 | Acinetobacter johnsonii SH046 genomic scaffold supercont1.16: NZ_GG704979 | 3.21441196797496e−37 |
| 35 | NC_011586 | Acinetobacter baumannii AB0057: NC_011586 | 4.65662453496211e−37 |
| 36 | NC_011586 | Acinetobacter baumannii AB0057: NC_011586 | 4.97462543315347e−37 |
| 37 | NC_010611 | Acinetobacter baumannii ACICU: NC_010611 | 5.86970772802613e−37 |
| 38 | CP002522 | Acinetobacter baumannii TCDC-AB0715: CP002522 | 5.86970772802613e−37 |
| 39 | NZ_ABXK01000021 | Acinetobacter baumannii AB900: NZ_ABXK01000021 | 7.97065298524188e−37 |
| 40 | CP001921 | Acinetobacter baumannii 1656-2: CP001921 | 8.02210311363916e−37 |
| 41 | NZ_ACYQ01000067 | Acinetobacter sp. 6013150 NZ_ACYQ01000067: NZ_ACYQ01000067 | 1.04988843673196e−36 |
| 42 | NZ_GG704579 | Acinetobacter baumannii ATCC 19606 genomic scaffold supercont1.8: | 2.27416603584469e−36 |
| | | NZ_GG704579 | |
| 43 | NC_010611 | Acinetobacter baumannii ACICU: NC_010611 | 2.41369225680075e−36 |
| 44 | NZ_ADMT01000124 | Acinetobacter haemolyticus ATCC 19194 contig00185: NZ_ADMT01000124 | 8.1885528903387e−36 |
| 45 | NC_011586 | Acinetobacter baumannii AB0057: NC_011586 | 8.6668830227109e−36 |
| 46 | CP002177 | Acinetobacter calcoaceticus PHEA-2: CP002177 | 1.06688951972352e−35 |
| 47 | NZ_GG704579 | Acinetobacter baumannii ATCC 19606 genomic scaffold supercont1.8: | 1.73119071134862e−35 |
| | | NZ_GG704579 | |
| 48 | NC_011595 | Acinetobacter baumannii AB307-0294: NC_011595 | 2.17923094612776e−35 |
| 49 | NZ_GG704499 | Acinetobacter sp. RUH2624 genomic scaffold supercont1.5: NZ_GG704499 | 2.56827101997287e−35 |
| 50 | CP002522 | Acinetobacter baumannii TCDC-AB0715: CP002522 | 2.89444275071491e−35 |

TABLE 1b
Data for Acinetobacter, particularly Acinetobacter baumanii (continued)

| No. | sign_phenos | sign_phenos_class | best_pheno | best_pheno_class |
|---|---|---|---|---|
| 1 | CP; GM; IMP; LVX; MER; TO; T/S | aminoglycoside; fluoroquinolone; lactam; other | CP | fluoroquinolone |
| 2 | CP; GM; IMP; LVX; MER; TO; T/S | aminoglycoside; fluoroquinolone; lactam; other | CP | fluoroquinolone |
| 3 | CP; GM; IMP; LVX; MER; TO; T/S | aminoglycoside; fluoroquinolone; lactam; other | CP | fluoroquinolone |
| 4 | CP; GM; IMP; LVX; MER; TO; T/S | aminoglycoside; fluoroquinolone; lactam; other | CP | fluoroquinolone |
| 5 | CP; GM; IMP; LVX; MER; TO; T/S | aminoglycoside; fluoroquinolone; lactam; other | CP | fluoroquinolone |
| 6 | CP; GM; IMP; LVX; MER; TO; T/S | aminoglycoside; fluoroquinolone; lactam; other | CP | fluoroquinolone |
| 7 | CP; GM; IMP; LVX; MER; TO; T/S | aminoglycoside; fluoroquinolone; lactam; other | CP | fluoroquinolone |
| 8 | CP; GM; IMP; LVX; MER; TO; T/S | aminoglycoside; fluoroquinolone; lactam; other | CP | fluoroquinolone |
| 9 | CP; GM; IMP; LVX; MER; TO; T/S | aminoglycoside; fluoroquinolone; lactam; other | CP | fluoroquinolone |
| 10 | CP; GM; IMP; LVX; MER; TO; T/S | aminoglycoside; fluoroquinolone; lactam; other | CP | fluoroquinolone |
| 11 | CP; GM; IMP; LVX; MER; TO; T/S | aminoglycoside; fluoroquinolone; lactam; other | CP | fluoroquinolone |
| 12 | CP; GM; IMP; LVX; MER; TO; T/S | aminoglycoside; fluoroquinolone; lactam; other | CP | fluoroquinolone |
| 13 | CP; GM; IMP; LVX; MER; TO; T/S | aminoglycoside; fluoroquinolone; lactam; other | CP | fluoroquinolone |
| 14 | CP; GM; IMP; LVX; MER; TO; T/S | aminoglycoside; fluoroquinolone; lactam; other | CP | fluoroquinolone |
| 15 | CP; GM; IMP; LVX; MER; TO; T/S | aminoglycoside; fluoroquinolone; lactam; other | CP | fluoroquinolone |
| 16 | CP; GM; IMP; LVX; MER; TO; T/S | aminoglycoside; fluoroquinolone; lactam; other | CP | fluoroquinolone |
| 17 | CP; GM; IMP; LVX; MER; TO; T/S | aminoglycoside; fluoroquinolone; lactam; other | CP | fluoroquinolone |
| 18 | CP; GM; IMP; LVX; MER; TO; T/S | aminoglycoside; fluoroquinolone; lactam; other | CP | fluoroquinolone |
| 19 | CP; GM; IMP; LVX; MER; TO; T/S | aminoglycoside; fluoroquinolone; lactam; other | CP | fluoroquinolone |
| 20 | CP; GM; IMP; LVX; MER; TO; T/S | aminoglycoside; fluoroquinolone; lactam; other | CP | fluoroquinolone |
| 21 | CP; GM; IMP; LVX; MER; TO; T/S | aminoglycoside; fluoroquinolone; lactam; other | CP | fluoroquinolone |
| 22 | CP; GM; IMP; LVX; MER; TO; T/S | aminoglycoside; fluoroquinolone; lactam; other | CP | fluoroquinolone |
| 23 | CP; GM; IMP; LVX; MER; TO; T/S | aminoglycoside; fluoroquinolone; lactam; other | CP | fluoroquinolone |
| 24 | CP; GM; IMP; LVX; MER; TO; T/S | aminoglycoside; fluoroquinolone; lactam; other | CP | fluoroquinolone |
| 25 | CP; GM; IMP; LVX; MER; TO; T/S | aminoglycoside; fluoroquinolone; lactam; other | LVX | fluoroquinolone |
| 26 | CP; GM; IMP; LVX; MER; TO; T/S | aminoglycoside; fluoroquinolone; lactam; other | CP | fluoroquinolone |
| 27 | CP; GM; IMP; LVX; MER; TO; T/S | aminoglycoside; fluoroquinolone; lactam; other | LVX | fluoroquinolone |
| 28 | CP; GM; IMP; LVX; MER; TO; T/S | aminoglycoside; fluoroquinolone; lactam; other | CP | fluoroquinolone |
| 29 | CP; GM; IMP; LVX; MER; TO; T/S | aminoglycoside; fluoroquinolone; lactam; other | LVX | fluoroquinolone |
| 30 | CP; GM; IMP; LVX; MER; TO; T/S | aminoglycoside; fluoroquinolone; lactam; other | CP | fluoroquinolone |
| 31 | CP; GM; IMP; LVX; MER; TO; T/S | aminoglycoside; fluoroquinolone; lactam; other | CP | fluoroquinolone |
| 32 | CP; GM; IMP; LVX; MER; TO; T/S | aminoglycoside; fluoroquinolone; lactam; other | CP | fluoroquinolone |
| 33 | CP; GM; IMP; LVX; MER; TO; T/S | aminoglycoside; fluoroquinolone; lactam; other | CP | fluoroquinolone |
| 34 | CP; GM; IMP; LVX; MER; TO; T/S | aminoglycoside; fluoroquinolone; lactam; other | CP | fluoroquinolone |
| 35 | CP; GM; IMP; LVX; MER; TO; T/S | aminoglycoside; fluoroquinolone; lactam; other | CP | fluoroquinolone |
| 36 | CP; GM; IMP; LVX; MER; TO; T/S | aminoglycoside; fluoroquinolone; lactam; other | CP | fluoroquinolone |
| 37 | CP; GM; IMP; LVX; MER; TO; T/S | aminoglycoside; fluoroquinolone; lactam; other | CP | fluoroquinolone |
| 38 | CP; GM; IMP; LVX; MER; TO; T/S | aminoglycoside; fluoroquinolone; lactam; other | CP | fluoroquinolone |
| 39 | CP; GM; IMP; LVX; MER; TO; T/S | aminoglycoside; fluoroquinolone; lactam; other | CP | fluoroquinolone |
| 40 | CP; GM; IMP; LVX; MER; TO; T/S | aminoglycoside; fluoroquinolone; lactam; other | LVX | fluoroquinolone |
| 41 | CP; GM; IMP; LVX; MER; TO; T/S | aminoglycoside; fluoroquinolone; lactam; other | CP | fluoroquinolone |
| 42 | CP; GM; IMP; LVX; MER; TO; T/S | aminoglycoside; fluoroquinolone; lactam; other | T/S | other |
| 43 | CP; GM; IMP; LVX; MER; TO; T/S | aminoglycoside; fluoroquinolone; lactam; other | LVX | fluoroquinolone |
| 44 | CP; GM; IMP; LVX; MER; TO; T/S | aminoglycoside; fluoroquinolone; lactam; other | LVX | fluoroquinolone |
| 45 | CP; GM; IMP; LVX; MER; TO; T/S | aminoglycoside; fluoroquinolone; lactam; other | CP | fluoroquinolone |
| 46 | CP; GM; IMP; LVX; MER; TO; T/S | aminoglycoside; fluoroquinolone; lactam; other | CP | fluoroquinolone |
| 47 | CP; GM; IMP; LVX; MER; TO; T/S | aminoglycoside; fluoroquinolone; lactam; other | T/S | other |
| 48 | CP; GM; IMP; LVX; MER; TO; T/S | aminoglycoside; fluoroquinolone; lactam; other | CP | fluoroquinolone |
| 49 | CP; GM; IMP; LVX; MER; TO; T/S | aminoglycoside; fluoroquinolone; lactam; other | CP | fluoroquinolone |
| 50 | CP; GM; IMP; LVX; MER; TO; T/S | aminoglycoside; fluoroquinolone; lactam; other | CP | fluoroquinolone |

TABLE 1c
Data for Acinetobacter, particularly Acinetobacter baumanii (continued)

| No. | num_aminoglycoside | num_fluoroquinolone | num_lactam | num_other |
|---|---|---|---|---|
| 1 | 2 | 2 | 2 | 1 |
| 2 | 2 | 2 | 2 | 1 |
| 3 | 2 | 2 | 2 | 1 |
| 4 | 2 | 2 | 2 | 1 |
| 5 | 2 | 2 | 2 | 1 |
| 6 | 2 | 2 | 2 | 1 |
| 7 | 2 | 2 | 2 | 1 |
| 8 | 2 | 2 | 2 | 1 |
| 9 | 2 | 2 | 2 | 1 |
| 10 | 2 | 2 | 2 | 1 |
| 11 | 2 | 2 | 2 | 1 |
| 12 | 2 | 2 | 2 | 1 |
| 13 | 2 | 2 | 2 | 1 |
| 14 | 2 | 2 | 2 | 1 |
| 15 | 2 | 2 | 2 | 1 |
| 16 | 2 | 2 | 2 | 1 |
| 17 | 2 | 2 | 2 | 1 |
| 18 | 2 | 2 | 2 | 1 |
| 19 | 2 | 2 | 2 | 1 |
| 20 | 2 | 2 | 2 | 1 |
| 21 | 2 | 2 | 2 | 1 |
| 22 | 2 | 2 | 2 | 1 |
| 23 | 2 | 2 | 2 | 1 |
| 24 | 2 | 2 | 2 | 1 |
| 25 | 2 | 2 | 2 | 1 |
| 26 | 2 | 2 | 2 | 1 |
| 27 | 2 | 2 | 2 | 1 |
| 28 | 2 | 2 | 2 | 1 |
| 29 | 2 | 2 | 2 | 1 |
| 30 | 2 | 2 | 2 | 1 |
| 31 | 2 | 2 | 2 | 1 |
| 32 | 2 | 2 | 2 | 1 |
| 33 | 2 | 2 | 2 | 1 |
| 34 | 2 | 2 | 2 | 1 |
| 35 | 2 | 2 | 2 | 1 |
| 36 | 2 | 2 | 2 | 1 |
| 37 | 2 | 2 | 2 | 1 |
| 38 | 2 | 2 | 2 | 1 |
| 39 | 2 | 2 | 2 | 1 |
| 40 | 2 | 2 | 2 | 1 |
| 41 | 2 | 2 | 2 | 1 |
| 42 | 2 | 2 | 2 | 1 |
| 43 | 2 | 2 | 2 | 1 |
| 44 | 2 | 2 | 2 | 1 |
| 45 | 2 | 2 | 2 | 1 |
| 46 | 2 | 2 | 2 | 1 |
| 47 | 2 | 2 | 2 | 1 |
| 48 | 2 | 2 | 2 | 1 |
| 49 | 2 | 2 | 2 | 1 |
| 50 | 2 | 2 | 2 | 1 |

TABLE 1d
Data for Acinetobacter, particularly Acinetobacter baumanii (continued)

| No. | CP_pv_adj | GM_pv_adj | IMP_pv_adj | LVX_pv_adj | MER_pv_adj | TO_pv_adj | T/S_pv_adj |
|---|---|---|---|---|---|---|---|
| 1 | 7.17E−34 | 7.62E−37 | 2.99E−16 | 2.51E−58 | 1.71E−29 | 3.85E−19 | 7.12E−37 |
| 2 | 1.39E−61 | 2.93E−36 | 2.52E−16 | 1.14E−57 | 1.26E−28 | 1.01E−18 | 2.48E−36 |
| 3 | 1.44E−56 | 1.41E−30 | 7.92E−24 | 2.33E−56 | 4.63E−35 | 1.23E−15 | 1.16E−31 |
| 4 | 5.42E−51 | 1.20E−26 | 3.83E−16 | 1.97E−49 | 3.77E−19 | 6.39E−14 | 5.70E−30 |
| 5 | 7.00E−47 | 7.36E−25 | 7.07E−15 | 3.32E−45 | 1.76E−24 | 2.16E−14 | 1.06E−32 |
| 6 | 2.06E−46 | 1.72E−23 | 1.92E−08 | 3.46E−46 | 2.40E−18 | 1.64E−13 | 3.34E−31 |
| 7 | 8.99E−46 | 1.85E−24 | 2.19E−15 | 2.93E−44 | 1.16E−24 | 3.33E−14 | 3.29E−31 |
| 8 | 2.77E−44 | 1.51E−23 | 2.19E−15 | 5.38E−43 | 1.16E−24 | 3.33E−14 | 3.45E−30 |
| 9 | 6.86E−43 | 1.15E−22 | 4.78E−15 | 1.09E−41 | 3.50E−24 | 5.85E−13 | 2.90E−29 |
| 10 | 5.21E−42 | 7.13E−19 | 3.88E−19 | 3.01E−41 | 3.46E−19 | 1.35E−06 | 1.20E−22 |
| 11 | 9.06E−42 | 1.74E−21 | 5.28E−11 | 6.20E−39 | 2.05E−18 | 9.48E−14 | 2.09E−27 |
| 12 | 9.06E−42 | 1.40E−20 | 1.57E−11 | 1.29E−40 | 2.50E−19 | 2.12E−13 | 1.47E−25 |
| 13 | 9.06E−42 | 1.40E−20 | 1.57E−11 | 1.29E−40 | 2.50E−19 | 2.12E−13 | 1.47E−25 |
| 14 | 9.06E−42 | 1.26E−22 | 4.71E−10 | 1.24E−38 | 1.59E−17 | 1.36E−14 | 1.16E−28 |
| 15 | 9.06E−42 | 1.40E−20 | 1.57E−11 | 1.29E−40 | 2.50E−19 | 2.12E−13 | 1.47E−25 |
| 16 | 9.06E−42 | 1.40E−20 | 1.57E−11 | 1.29E−40 | 2.50E−19 | 2.12E−13 | 1.47E−25 |
| 17 | 9.06E−42 | 1.40E−20 | 1.57E−11 | 1.29E−40 | 2.50E−19 | 2.12E−13 | 1.47E−25 |
| 18 | 3.84E−41 | 4.26E−21 | 5.48E−11 | 2.25E−38 | 2.28E−18 | 1.85E−13 | 1.34E−28 |
| 19 | 3.90E−41 | 3.00E−22 | 8.17E−10 | 4.60E−38 | 3.07E−17 | 2.60E−14 | 3.05E−28 |
| 20 | 1.29E−40 | 2.54E−23 | 8.32E−09 | 2.28E−37 | 2.98E−16 | 7.17E−15 | 4.45E−29 |
| 21 | 1.49E−40 | 1.06E−20 | 9.78E−11 | 8.02E−38 | 4.26E−18 | 3.58E−13 | 1.41E−26 |
| 22 | 7.70E−40 | 2.19E−21 | 2.55E−13 | 8.73E−40 | 2.71E−22 | 1.30E−12 | 9.73E−29 |
| 23 | 9.97E−40 | 1.94E−20 | 5.69E−10 | 6.32E−37 | 5.09E−15 | 3.42E−13 | 4.53E−25 |
| 24 | 1.66E−39 | 2.25E−21 | 1.11E−11 | 4.74E−37 | 9.11E−19 | 4.30E−12 | 6.58E−28 |
| 25 | 6.97E−38 | 2.51E−23 | 4.39E−17 | 2.08E−39 | 2.11E−31 | 2.81E−20 | 2.62E−29 |
| 26 | 6.41E−39 | 8.82E−20 | 8.32E−09 | 2.28E−37 | 8.45E−15 | 5.48E−12 | 1.80E−23 |
| 27 | 1.21E−36 | 6.79E−22 | 2.00E−18 | 1.22E−38 | 1.37E−30 | 1.91E−20 | 1.86E−29 |
| 28 | 2.20E−38 | 6.34E−16 | 1.51E−04 | 3.00E−37 | 3.02E−15 | 2.23E−05 | 5.58E−26 |
| 29 | 2.16E−37 | 5.62E−21 | 1.99E−11 | 5.31E−38 | 4.07E−20 | 1.62E−14 | 1.79E−33 |
| 30 | 6.26E−38 | 1.31E−11 | 3.04E−05 | 7.55E−37 | 2.46E−19 | 5.99E−05 | 3.36E−19 |
| 31 | 1.70E−37 | 9.89E−20 | 4.00E−15 | 6.17E−33 | 1.59E−19 | 1.06E−07 | 5.60E−19 |
| 32 | 2.25E−37 | 3.62E−20 | 5.26E−16 | 3.31E−34 | 9.82E−23 | 4.38E−09 | 5.82E−22 |
| 33 | 2.44E−37 | 6.97E−16 | 4.33E−14 | 7.77E−37 | 9.30E−19 | 4.18E−07 | 1.39E−20 |
| 34 | 3.21E−37 | 1.12E−20 | 1.05E−11 | 3.70E−36 | 1.02E−20 | 3.75E−13 | 1.16E−24 |
| 35 | 4.66E−37 | 5.92E−20 | 3.00E−11 | 4.04E−32 | 8.17E−17 | 5.55E−10 | 2.22E−21 |
| 36 | 4.97E−37 | 9.12E−20 | 3.90E−11 | 7.27E−35 | 8.20E−18 | 2.95E−11 | 2.78E−26 |
| 37 | 5.87E−37 | 1.44E−15 | 3.36E−14 | 2.00E−36 | 3.55E−19 | 3.60E−07 | 3.09E−20 |
| 38 | 5.87E−37 | 1.44E−15 | 2.29E−13 | 2.00E−36 | 2.38E−18 | 1.01E−06 | 4.30E−21 |
| 39 | 7.97E−37 | 1.41E−20 | 4.11E−10 | 1.69E−34 | 6.22E−17 | 1.57E−12 | 9.61E−26 |
| 40 | 8.02E−36 | 1.02E−16 | 8.36E−13 | 8.02E−37 | 6.77E−18 | 2.61E−10 | 1.91E−19 |
| 41 | 1.05E−36 | 5.82E−22 | 6.09E−07 | 1.09E−33 | 3.93E−12 | 9.82E−13 | 1.13E−26 |
| 42 | 2.17E−18 | 1.05E−13 | 2.19E−05 | 7.67E−17 | 1.44E−05 | 1.58E−19 | 2.27E−36 |
| 43 | 2.04E−35 | 2.18E−16 | 4.05E−13 | 2.41E−36 | 4.89E−18 | 7.30E−10 | 2.59E−18 |
| 44 | 2.30E−34 | 4.36E−15 | 1.44E−11 | 8.19E−36 | 1.06E−16 | 9.26E−09 | 1.67E−18 |
| 45 | 8.67E−36 | 6.11E−17 | 3.23E−06 | 1.48E−32 | 1.25E−11 | 1.06E−09 | 8.79E−25 |
| 46 | 1.07E−35 | 6.07E−21 | 9.46E−09 | 3.15E−33 | 4.04E−15 | 6.09E−14 | 1.52E−24 |
| 47 | 2.15E−17 | 3.02E−13 | 2.37E−05 | 3.26E−16 | 2.56E−05 | 2.12E−19 | 1.73E−35 |
| 48 | 2.18E−35 | 2.69E−18 | 1.65E−04 | 2.17E−32 | 2.13E−10 | 9.38E−10 | 2.81E−21 |
| 49 | 2.57E−35 | 5.01E−23 | 1.62E−06 | 3.27E−32 | 1.19E−11 | 4.78E−13 | 2.98E−26 |
| 50 | 2.89E−35 | 4.58E−19 | 5.87E−06 | 4.77E−32 | 5.92E−13 | 2.05E−11 | 8.30E−23 |

TABLE 1e
Data for Acinetobacter, particularly Acinetobacter baumanii (continued)

| | | | | |
|---|---|---|---|---|
| No. | Locus.Tag | Gene.Symbol | GenBank.Accession | Chromosome |

| | | | |
|---|---|---|---|
| 1 | ABK1_1135 | | ADX02769 |
| 2 | A60131_010100011170 | | ZP_06783174 |
| 3 | A1S_0690 | | YP_001083736 |
| 4 | ABBFA_001710 | | YP_002325613 |
| 5 | ABBFA_001640 | | YP_002325543 |
| 6 | AB57_1876 | | YP_002319238 |
| 7 | AB57_2151 | | YP_002319507 |
| 8 | ABBFA_001638 | | YP_002325541 |
| 9 | AB57_2147 | | YP_002319503 |
| 10 | AbauAB_010100009672 | | ZP_04661877 |
| 11 | HMPREF0014_00181 | | ZP_05823167 |
| 12 | HMPREF0014_02450 | | ZP_05825436 |
| 13 | HMPREF0014_02452 | | ZP_05825438 |
| 14 | AOLE_17735 | | YP_003733805 |
| 15 | HMPREF0012_01352 | | ZP_06057184 |
| 16 | HMPREF0014_02453 | | ZP_05825439 |
| 17 | BDGL_001498 | ocd2 | ADY82084 |
| 18 | HMPREF0014_02489 | | ZP_05825475 |
| 19 | HMPREF0014_00004 | | ZP_05822990 |
| 20 | ACIAD0370 | rbfA | YP_045140 |
| 21 | HMPREF0014_02492 | | ZP_05825478 |
| 22 | ABBFA_001637 | | YP_002325540 |
| 23 | ABTW07_1894 | | ADX92323 |
| 24 | ABBFA_000343 | | YP_002324276 |
| 25 | AB57_2771 | | YP_002320110 |
| 26 | AOLE_17690 | | YP_003733796 |
| 27 | HMPREF0017_03097 | | ZP_06071176 |
| 28 | A6013_010100003618 | | ZP_06795427 |
| 29 | HMPREF0014_03338 | | ZP_05826324 |
| 30 | A6014_010100015962 | | ZP_06788050 |
| 31 | ACICU_00639 | filE | YP_001845298 |
| 32 | HMPREF0013_02137 | | ZP_06692288 |
| 33 | ABTW07_2865 | | ADX93289 |
| 34 | HMPREF0016_03291 | | ZP_06064821 |
| 35 | AB57_2131 | | YP_002319487 |
| 36 | AB57_3624 | pilV | YP_002320921 |
| 37 | ACICU_02614 | | YP_001847273 |
| 38 | ABTW07_2863 | | ADX93287 |
| 39 | AbauAB_010100013685 | | ZP_04662667 |
| 40 | ABK1_2278 | | ADX03912 |
| 41 | A6013_010100009420 | | ZP_06796568 |
| 42 | HMPREF0010_03320 | | ZP_05829937 |
| 43 | ACICU_01819 | | YP_001846478 |
| 44 | HMP0015_1294 | | ZP_06727085 |
| 45 | AB57_1430 | | YP_002318806 |
| 46 | BDGL_000595 | | ADY81181 |
| 47 | HMPREF0010_03319 | | ZP_05829936 |
| 48 | ABBFA_001847 | | YP_002325750 |
| 49 | HMPREF0014_00860 | | ZP_05823846 |
| 50 | ABTW07_1891 | | ADX92320 |

TABLE 1f
Data for Acinetobacter, particularly
Acinetobacter baumanii (continued)

| No. | Start.Coord | End.Coord | Strand | Scaffold.ID |
|---|---|---|---|---|
| 1 | 1235814 | 1236425 | + | 650377914 |
| 2 | 64 | 768 | + | 647008664 |
| 3 | 821275 | 822057 | + | 640069344 |
| 4 | 1834753 | 1835667 | − | 643348583 |
| 5 | 1759838 | 1761280 | + | 643348583 |
| 6 | 1966831 | 1969149 | + | 643348581 |
| 7 | 2232790 | 2233716 | − | 643348581 |
| 8 | 1757930 | 1758571 | − | 643348583 |
| 9 | 2226774 | 2228099 | + | 643348581 |
| 10 | 63870 | 65015 | − | 645061599 |
| 11 | 195165 | 195419 | + | 647535325 |
| 12 | 27528 | 28301 | − | 647535345 |
| 13 | 31818 | 33023 | − | 647535345 |
| 14 | 3783514 | 3783789 | + | 648028017 |
| 15 | 1494626 | 1495630 | + | 646207323 |
| 16 | 33027 | 34796 | − | 647535345 |
| 17 | 1603798 | 1604811 | + | 650377930 |
| 18 | 74534 | 74893 | + | 647535345 |
| 19 | 1819 | 2325 | − | 647535325 |
| 20 | 363575 | 363976 | + | 637000277 |
| 21 | 76454 | 76681 | + | 647535345 |
| 22 | 1757594 | 1757890 | − | 643348583 |
| 23 | 2004366 | 2004983 | + | 650378014 |
| 24 | 370977 | 371978 | + | 643348583 |
| 25 | 2838051 | 2838620 | − | 643348581 |
| 26 | 3773081 | 3773467 | + | 648028017 |
| 27 | 716 | 1162 | + | 646207467 |
| 28 | 6958 | 7416 | − | 647008417 |
| 29 | 12541 | 13101 | − | 647535362 |
| 30 | 7588 | 7713 | + | 647009002 |
| 31 | 705957 | 707216 | + | 642555129 |
| 32 | 856694 | 857281 | − | 647536755 |
| 33 | 3040319 | 3041707 | − | 650378014 |
| 34 | 4553 | 4768 | − | 646207353 |
| 35 | 2213458 | 2214801 | − | 643348581 |
| 36 | 3730668 | 3731222 | − | 643348581 |
| 37 | 2775538 | 2776941 | + | 642555129 |
| 38 | 3038397 | 3039893 | − | 650378014 |
| 39 | 70983 | 71513 | − | 645061607 |
| 40 | 2403829 | 2405136 | + | 650377914 |
| 41 | 2603 | 3454 | + | 647008460 |
| 42 | 159696 | 159998 | + | 647535409 |
| 43 | 1941428 | 1942675 | + | 642555129 |
| 44 | 1088 | 2629 | − | 647018944 |
| 45 | 1505450 | 1506382 | + | 643348581 |
| 46 | 622112 | 622732 | + | 650377930 |
| 47 | 158794 | 159609 | + | 647535409 |
| 48 | 1980078 | 1981988 | − | 643348583 |
| 49 | 126664 | 127845 | + | 647535329 |
| 50 | 1999835 | 2001676 | + | 650378014 |

TABLE 2a
Data for Acinetobacter, particularly Acinetobacter baumanii

| No. | Scaffold.External.Accession | Scaffold.Name | best_pv |
|---|---|---|---|
| 1 | CP001921 | Acinetobacter baumannii 1656-2: CP001921 | 7.16783907123597e−62 |
| 2 | NZ_ACYR01000063 | Acinetobacter sp. 6013113 NZ_ACYR01000063: | 1.39202289199418e−61 |
| | | NZ_ACYR01000063 | |
| 3 | NC_009085 | Acinetobacter baumannii ATCC 17978: NC_009085 | 1.43720401820086e−56 |
| 4 | NC_011595 | Acinetobacter baumannii AB307-0294: NC_011595 | 5.41948460225177e−51 |
| 5 | NC_011595 | Acinetobacter baumannii AB307-0294: NC_011595 | 7.00413054521082e−47 |
| 6 | NC_011586 | Acinetobacter baumannii AB0057: NC_011586 | 2.06435524702153e−46 |
| 7 | NC_011586 | Acinetobacter baumannii AB0057: NC_011586 | 8.98962816077673e−46 |
| 8 | NC_011595 | Acinetobacter baumannii AB307-0294: NC_011595 | 2.76883973579195e−44 |
| 9 | NC_011586 | Acinetobacter baumannii AB0057: NC_011586 | 6.86281420221907e−43 |
| 10 | NZ_ABXK01000013 | Acinetobacter baumannii AB900: NZ_ABXK01000013 | 5.20862955909241e−42 |
| 11 | NZ_GG704495 | Acinetobacter sp. RUH2624 genomic scaffold | 9.06181183555984e−42 |
| | | supercont1.1: NZ_GG704495 | |
| 12 | NZ_GG704515 | Acinetobacter sp. RUH2624 genomic scaffold | 9.06181183555984e−42 |
| | | supercont1.21: NZ_GG704515 | |
| 13 | NZ_GG704515 | Acinetobacter sp. RUH2624 genomic scaffold | 9.06181183555984e−42 |
| | | supercont1.21: NZ_GG704515 | |
| 14 | NC_014259 | Acinetobacter sp. DR1 chromosome: NC_014259 | 9.06181183555984e−42 |
| 15 | NZ_GG704949 | Acinetobacter calcoaceticus RUH2202 genomic | 9.06181183555984e−42 |
| | | scaffold supercont1.1: NZ_GG704949 | |
| 16 | NZ_GG704515 | Acinetobacter sp. RUH2624 genomic scaffold | 9.06181183555984e−42 |
| | | supercont1.21: NZ_GG704515 | |
| 17 | CP002177 | Acinetobacter calcoaceticus PHEA-2: CP002177 | 9.06181183555984e−42 |
| 18 | NZ_GG704515 | Acinetobacter sp. RUH2624 genomic scaffold | 3.84309843417235e−41 |
| | | supercont1.21: NZ_GG704515 | |
| 19 | NZ_GG704495 | Acinetobacter sp. RUH2624 genomic scaffold | 3.89818602393582e−41 |
| | | supercont1.1: NZ_GG704495 | |
| 20 | NC_005966 | Acinetobacter sp. ADP1: NC_005966 | 1.28694655921313e−40 |
| 21 | NZ_GG704515 | Acinetobacter sp. RUH2624 genomic scaffold | 1.49093940399756e−40 |
| | | supercont1.21: NZ_GG704515 | |
| 22 | NC_011595 | Acinetobacter baumannii AB307-0294: NC_011595 | 7.70079013246134e−40 |
| 23 | CP002522 | Acinetobacter baumannii TCDC-AB0715: CP002522 | 9.96749396638303e−40 |
| 24 | NC_011595 | Acinetobacter baumannii AB307-0294: NC_011595 | 1.66049576626689e−39 |
| 25 | NC_011586 | Acinetobacter baumannii AB0057: NC_011586 | 2.08472354496451e−39 |
| 26 | NC_014259 | Acinetobacter sp. DR1 chromosome: NC_014259 | 6.40954171412503e−39 |
| 27 | NZ_GG705093 | Acinetobacter lwoffii SH145 genomic scaffold | 1.2155660857651e−38 |
| | | supercont1.39: NZ_GG705093 | |
| 28 | NZ_ACYQ01000024 | Acinetobacter sp. 6013150 NZ_ACYQ01000024: | 2.19910980623625e−38 |
| | | NZ_ACYQ01000024 | |
| 29 | NZ_GG704532 | Acinetobacter sp. RUH2624 genomic scaffold | 5.31361687748575e−38 |
| | | supercont1.38: NZ_GG704532 | |
| 30 | NZ_ACYS01000159 | Acinetobacter sp. 6014059 NZ_ACYS01000159: | 6.25755791192393e−38 |
| | | NZ_ACYS01000159 | |
| 31 | NC_010611 | Acinetobacter baumannii ACICU: NC_010611 | 1.6998208344511e−37 |
| 32 | NZ_GG753601 | Acinetobacter sp. SH024 genomic scaffold | 2.25174704599278e−37 |
| | | supercont1.2: NZ_GG753601 | |
| 33 | CP002522 | Acinetobacter baumannii TCDC-AB0715: CP002522 | 2.43979456355917e−37 |
| 34 | NZ_GG704979 | Acinetobacter johnsonii SH046 genomic scaffold | 3.21441196797496e−37 |
| | | supercont1.16: NZ_GG704979 | |
| 35 | NC_011586 | Acinetobacter baumannii AB0057: NC_011586 | 4.65662453496211e−37 |
| 36 | NC_011586 | Acinetobacter baumannii AB0057: NC_011586 | 4.97462543315347e−37 |
| 37 | NC_010611 | Acinetobacter baumannii ACICU: NC_010611 | 5.86970772802613e−37 |
| 38 | CP002522 | Acinetobacter baumannii TCDC-AB0715: CP002522 | 5.86970772802613e−37 |
| 39 | NZ_ABXK01000021 | Acinetobacter baumannii AB900: NZ_ABXK01000021 | 7.97065298524188e−37 |
| 40 | CP001921 | Acinetobacter baumannii 1656-2: CP001921 | 8.02210311363916e−37 |
| 41 | NZ_ACYQ01000067 | Acinetobacter sp. 6013150 NZ_ACYQ01000067: | 1.04988843673196e−36 |
| | | NZ_ACYQ01000067 | |
| 42 | NZ_GG704579 | Acinetobacter baumannii ATCC 19606 genomic | 2.27416603584469e−36 |
| | | scaffold supercont1.8: NZ_GG704579 | |
| 43 | NC_010611 | Acinetobacter baumannii ACICU: NC_010611 | 2.41369225680075e−36 |
| 44 | NZ_ADMT01000124 | Acinetobacter haemolyticus ATCC 19194 | 8.1885528903387e−36 |
| | | contig00185: NZ_ADMT01000124 | |
| 45 | NC_011586 | Acinetobacter baumannii AB0057: NC_011586 | 8.6668830227109e−36 |
| 46 | CP002177 | Acinetobacter calcoaceticus PHEA-2: CP002177 | 1.06688951972352e−35 |
| 47 | NZ_GG704579 | Acinetobacter baumannii ATCC 19606 genomic | 1.73119071134862e−35 |
| | | scaffold supercont1.8: NZ_GG704579 | |
| 48 | NC_011595 | Acinetobacter baumannii AB307-0294: NC_011595 | 2.17923094612776e−35 |
| 49 | NZ_GG704499 | Acinetobacter sp. RUH2624 genomic scaffold | 2.56827101997287e−35 |
| | | supercont1.5: NZ_GG704499 | |
| 50 | CP002522 | Acinetobacter baumannii TCDC-AB0715: CP002522 | 2.89444275071491e−35 |

TABLE 2b
Data for Acinetobacter, particularly Acinetobacter baumanii (continued)

| No. | sign_phenos | sign_phenos_class | best_pheno | best_pheno_class |
|---|---|---|---|---|
| 1 | CP; GM; IMP; LVX; MER; TO; T/S | aminoglycoside; fluoroquinolone; lactam; other | CP | fluoroquinolone |
| 2 | CP; GM; IMP; LVX; MER; TO; T/S | aminoglycoside; fluoroquinolone; lactam; other | CP | fluoroquinolone |
| 3 | CP; GM; IMP; LVX; MER; TO; T/S | aminoglycoside; fluoroquinolone; lactam; other | CP | fluoroquinolone |
| 4 | CP; GM; IMP; LVX; MER; TO; T/S | aminoglycoside; fluoroquinolone; lactam; other | CP | fluoroquinolone |
| 5 | CP; GM; IMP; LVX; MER; TO; T/S | aminoglycoside; fluoroquinolone; lactam; other | CP | fluoroquinolone |
| 6 | CP; GM; IMP; LVX; MER; TO; T/S | aminoglycoside; fluoroquinolone; lactam; other | CP | fluoroquinolone |
| 7 | CP; GM; IMP; LVX; MER; TO; T/S | aminoglycoside; fluoroquinolone; lactam; other | CP | fluoroquinolone |
| 8 | CP; GM; IMP; LVX; MER; TO; T/S | aminoglycoside; fluoroquinolone; lactam; other | CP | fluoroquinolone |
| 9 | CP; GM; IMP; LVX; MER; TO; T/S | aminoglycoside; fluoroquinolone; lactam; other | CP | fluoroquinolone |
| 10 | CP; GM; IMP; LVX; MER; TO; T/S | aminoglycoside; fluoroquinolone; lactam; other | CP | fluoroquinolone |
| 11 | CP; GM; IMP; LVX; MER; TO; T/S | aminoglycoside; fluoroquinolone; lactam; other | CP | fluoroquinolone |
| 12 | CP; GM; IMP; LVX; MER; TO; T/S | aminoglycoside; fluoroquinolone; lactam; other | CP | fluoroquinolone |
| 13 | CP; GM; IMP; LVX; MER; TO; T/S | aminoglycoside; fluoroquinolone; lactam; other | CP | fluoroquinolone |
| 14 | CP; GM; IMP; LVX; MER; TO; T/S | aminoglycoside; fluoroquinolone; lactam; other | CP | fluoroquinolone |
| 15 | CP; GM; IMP; LVX; MER; TO; T/S | aminoglycoside; fluoroquinolone; lactam; other | CP | fluoroquinolone |
| 16 | CP; GM; IMP; LVX; MER; TO; T/S | aminoglycoside; fluoroquinolone; lactam; other | CP | fluoroquinolone |
| 17 | CP; GM; IMP; LVX; MER; TO; T/S | aminoglycoside; fluoroquinolone; lactam; other | CP | fluoroquinolone |
| 18 | CP; GM; IMP; LVX; MER; TO; T/S | aminoglycoside; fluoroquinolone; lactam; other | CP | fluoroquinolone |
| 19 | CP; GM; IMP; LVX; MER; TO; T/S | aminoglycoside; fluoroquinolone; lactam; other | CP | fluoroquinolone |
| 20 | CP; GM; IMP; LVX; MER; TO; T/S | aminoglycoside; fluoroquinolone; lactam; other | CP | fluoroquinolone |
| 21 | CP; GM; IMP; LVX; MER; TO; T/S | aminoglycoside; fluoroquinolone; lactam; other | CP | fluoroquinolone |
| 22 | CP; GM; IMP; LVX; MER; TO; T/S | aminoglycoside; fluoroquinolone; lactam; other | CP | fluoroquinolone |
| 23 | CP; GM; IMP; LVX; MER; TO; T/S | aminoglycoside; fluoroquinolone; lactam; other | CP | fluoroquinolone |
| 24 | CP; GM; IMP; LVX; MER; TO; T/S | aminoglycoside; fluoroquinolone; lactam; other | CP | fluoroquinolone |
| 25 | CP; GM; IMP; LVX; MER; TO; T/S | aminoglycoside; fluoroquinolone; lactam; other | LVX | fluoroquinolone |
| 26 | CP; GM; IMP; LVX; MER; TO; T/S | aminoglycoside; fluoroquinolone; lactam; other | CP | fluoroquinolone |
| 27 | CP; GM; IMP; LVX; MER; TO; T/S | aminoglycoside; fluoroquinolone; lactam; other | LVX | fluoroquinolone |
| 28 | CP; GM; IMP; LVX; MER; TO; T/S | aminoglycoside; fluoroquinolone; lactam; other | CP | fluoroquinolone |
| 29 | CP; GM; IMP; LVX; MER; TO; T/S | aminoglycoside; fluoroquinolone; lactam; other | LVX | fluoroquinolone |
| 30 | CP; GM; IMP; LVX; MER; TO; T/S | aminoglycoside; fluoroquinolone; lactam; other | CP | fluoroquinolone |
| 31 | CP; GM; IMP; LVX; MER; TO; T/S | aminoglycoside; fluoroquinolone; lactam; other | CP | fluoroquinolone |
| 32 | CP; GM; IMP; LVX; MER; TO; T/S | aminoglycoside; fluoroquinolone; lactam; other | CP | fluoroquinolone |
| 33 | CP; GM; IMP; LVX; MER; TO; T/S | aminoglycoside; fluoroquinolone; lactam; other | CP | fluoroquinolone |
| 34 | CP; GM; IMP; LVX; MER; TO; T/S | aminoglycoside; fluoroquinolone; lactam; other | CP | fluoroquinolone |
| 35 | CP; GM; IMP; LVX; MER; TO; T/S | aminoglycoside; fluoroquinolone; lactam; other | CP | fluoroquinolone |
| 36 | CP; GM; IMP; LVX; MER; TO; T/S | aminoglycoside; fluoroquinolone; lactam; other | CP | fluoroquinolone |
| 37 | CP; GM; IMP; LVX; MER; TO; T/S | aminoglycoside; fluoroquinolone; lactam; other | CP | fluoroquinolone |
| 38 | CP; GM; IMP; LVX; MER; TO; T/S | aminoglycoside; fluoroquinolone; lactam; other | CP | fluoroquinolone |
| 39 | CP; GM; IMP; LVX; MER; TO; T/S | aminoglycoside; fluoroquinolone; lactam; other | CP | fluoroquinolone |
| 40 | CP; GM; IMP; LVX; MER; TO; T/S | aminoglycoside; fluoroquinolone; lactam; other | LVX | fluoroquinolone |
| 41 | CP; GM; IMP; LVX; MER; TO; T/S | aminoglycoside; fluoroquinolone; lactam; other | CP | fluoroquinolone |
| 42 | CP; GM; IMP; LVX; MER; TO; T/S | aminoglycoside; fluoroquinolone; lactam; other | T/S | other |
| 43 | CP; GM; IMP; LVX; MER; TO; T/S | aminoglycoside; fluoroquinolone; lactam; other | LVX | fluoroquinolone |
| 44 | CP; GM; IMP; LVX; MER; TO; T/S | aminoglycoside; fluoroquinolone; lactam; other | LVX | fluoroquinolone |
| 45 | CP; GM; IMP; LVX; MER; TO; T/S | aminoglycoside; fluoroquinolone; lactam; other | CP | fluoroquinolone |
| 46 | CP; GM; IMP; LVX; MER; TO; T/S | aminoglycoside; fluoroquinolone; lactam; other | CP | fluoroquinolone |
| 47 | CP; GM; IMP; LVX; MER; TO; T/S | aminoglycoside; fluoroquinolone; lactam; other | T/S | other |
| 48 | CP; GM; IMP; LVX; MER; TO; T/S | aminoglycoside; fluoroquinolone; lactam; other | CP | fluoroquinolone |
| 49 | CP; GM; IMP; LVX; MER; TO; T/S | aminoglycoside; fluoroquinolone; lactam; other | CP | fluoroquinolone |
| 50 | CP; GM; IMP; LVX; MER; TO; T/S | aminoglycoside; fluoroquinolone; lactam; other | CP | fluoroquinolone |

TABLE 2c
Data for Acinetobacter, particularly Acinetobacter baumanii (continued)

| No. | num_aminoglycoside | num_fluoroquinolone | num_lactam | num_other |
|---|---|---|---|---|
| 1 | 2 | 2 | 2 | 1 |
| 2 | 2 | 2 | 2 | 1 |
| 3 | 2 | 2 | 2 | 1 |
| 4 | 2 | 2 | 2 | 1 |
| 5 | 2 | 2 | 2 | 1 |
| 6 | 2 | 2 | 2 | 1 |
| 7 | 2 | 2 | 2 | 1 |
| 8 | 2 | 2 | 2 | 1 |
| 9 | 2 | 2 | 2 | 1 |
| 10 | 2 | 2 | 2 | 1 |
| 11 | 2 | 2 | 2 | 1 |
| 12 | 2 | 2 | 2 | 1 |
| 13 | 2 | 2 | 2 | 1 |
| 14 | 2 | 2 | 2 | 1 |
| 15 | 2 | 2 | 2 | 1 |
| 16 | 2 | 2 | 2 | 1 |
| 17 | 2 | 2 | 2 | 1 |
| 18 | 2 | 2 | 2 | 1 |
| 19 | 2 | 2 | 2 | 1 |
| 20 | 2 | 2 | 2 | 1 |
| 21 | 2 | 2 | 2 | 1 |
| 22 | 2 | 2 | 2 | 1 |
| 23 | 2 | 2 | 2 | 1 |
| 24 | 2 | 2 | 2 | 1 |
| 25 | 2 | 2 | 2 | 1 |
| 26 | 2 | 2 | 2 | 1 |
| 27 | 2 | 2 | 2 | 1 |
| 28 | 2 | 2 | 2 | 1 |
| 29 | 2 | 2 | 2 | 1 |
| 30 | 2 | 2 | 2 | 1 |
| 31 | 2 | 2 | 2 | 1 |
| 32 | 2 | 2 | 2 | 1 |
| 33 | 2 | 2 | 2 | 1 |
| 34 | 2 | 2 | 2 | 1 |
| 35 | 2 | 2 | 2 | 1 |
| 36 | 2 | 2 | 2 | 1 |
| 37 | 2 | 2 | 2 | 1 |
| 38 | 2 | 2 | 2 | 1 |
| 39 | 2 | 2 | 2 | 1 |
| 40 | 2 | 2 | 2 | 1 |
| 41 | 2 | 2 | 2 | 1 |
| 42 | 2 | 2 | 2 | 1 |
| 43 | 2 | 2 | 2 | 1 |
| 44 | 2 | 2 | 2 | 1 |
| 45 | 2 | 2 | 2 | 1 |
| 46 | 2 | 2 | 2 | 1 |
| 47 | 2 | 2 | 2 | 1 |
| 48 | 2 | 2 | 2 | 1 |
| 49 | 2 | 2 | 2 | 1 |
| 50 | 2 | 2 | 2 | 1 |

TABLE 2d
Data for Acinetobacter, particularly Acinetobacter baumanii (continued)

| No. | CP_pv_adj | GM_pv_adj | IMP_pv_adj | LVX_pv_adj | MER_pv_adj | TO_pv_adj | T/S_pv_adj |
|---|---|---|---|---|---|---|---|
| 1 | 7.17E−62 | 7.62E−37 | 2.99E−16 | 2.51E−58 | 1.71E−29 | 3.85E−19 | 7.12E−37 |
| 2 | 1.39E−61 | 2.93E−36 | 2.52E−16 | 1.14E−57 | 1.26E−28 | 1.01E−18 | 2.48E−36 |
| 3 | 1.44E−56 | 1.41E−30 | 7.92E−24 | 2.33E−56 | 4.63E−35 | 1.23E−15 | 1.16E−31 |
| 4 | 5.42E−51 | 1.20E−26 | 3.83E−16 | 1.97E−49 | 3.77E−19 | 6.39E−14 | 5.70E−30 |
| 5 | 7.00E−47 | 7.36E−25 | 7.07E−15 | 3.32E−45 | 1.76E−24 | 2.16E−14 | 1.06E−32 |
| 6 | 2.06E−46 | 1.72E−23 | 1.92E−08 | 3.46E−46 | 2.40E−18 | 1.64E−13 | 3.34E−31 |
| 7 | 8.99E−46 | 1.85E−24 | 2.19E−15 | 2.93E−44 | 1.16E−24 | 3.33E−14 | 3.29E−31 |
| 8 | 2.77E−44 | 1.51E−23 | 2.19E−15 | 5.38E−43 | 1.16E−24 | 3.33E−14 | 3.45E−30 |
| 9 | 6.86E−43 | 1.15E−22 | 4.78E−15 | 1.09E−41 | 3.50E−24 | 5.85E−13 | 2.90E−29 |
| 10 | 5.21E−42 | 7.13E−19 | 3.88E−19 | 3.01E−41 | 3.46E−19 | 1.35E−06 | 1.20E−22 |
| 11 | 9.06E−42 | 1.74E−21 | 5.28E−11 | 6.20E−39 | 2.05E−18 | 9.48E−14 | 2.09E−27 |
| 12 | 9.06E−42 | 1.40E−20 | 1.57E−11 | 1.29E−40 | 2.50E−19 | 2.12E−13 | 1.47E−25 |
| 13 | 9.06E−42 | 1.40E−20 | 1.57E−11 | 1.29E−40 | 2.50E−19 | 2.12E−13 | 1.47E−25 |
| 14 | 9.06E−42 | 1.26E−22 | 4.71E−10 | 1.24E−38 | 1.59E−17 | 1.36E−14 | 1.16E−28 |
| 15 | 9.06E−42 | 1.40E−20 | 1.57E−11 | 1.29E−40 | 2.50E−19 | 2.12E−13 | 1.47E−25 |
| 16 | 9.06E−42 | 1.40E−20 | 1.57E−11 | 1.29E−40 | 2.50E−19 | 2.12E−13 | 1.47E−25 |
| 17 | 9.06E−42 | 1.40E−20 | 1.57E−11 | 1.29E−40 | 2.50E−19 | 2.12E−13 | 1.47E−25 |
| 18 | 3.84E−41 | 4.26E−21 | 5.48E−11 | 2.25E−38 | 2.28E−18 | 1.85E−13 | 1.34E−28 |
| 19 | 3.90E−41 | 3.00E−22 | 8.17E−10 | 4.60E−38 | 3.07E−17 | 2.60E−14 | 3.05E−28 |
| 20 | 1.29E−40 | 2.54E−23 | 8.32E−09 | 2.28E−37 | 2.98E−16 | 7.17E−15 | 4.45E−29 |
| 21 | 1.49E−40 | 1.06E−20 | 9.78E−11 | 8.02E−38 | 4.26E−18 | 3.58E−13 | 1.41E−26 |
| 22 | 7.70E−40 | 2.19E−21 | 2.55E−13 | 8.73E−40 | 2.71E−22 | 1.30E−12 | 9.73E−29 |
| 23 | 9.97E−40 | 1.94E−20 | 5.69E−10 | 6.32E−37 | 5.09E−15 | 3.42E−13 | 4.53E−25 |
| 24 | 1.66E−39 | 2.25E−21 | 1.11E−11 | 4.74E−37 | 9.11E−19 | 4.30E−12 | 6.58E−28 |
| 25 | 6.97E−38 | 2.51E−23 | 4.39E−17 | 2.08E−39 | 2.11E−31 | 2.81E−20 | 2.62E−29 |
| 26 | 6.41E−39 | 8.82E−20 | 8.32E−09 | 2.28E−37 | 8.45E−15 | 5.48E−12 | 1.80E−23 |
| 27 | 1.21E−36 | 6.79E−22 | 2.00E−18 | 1.22E−38 | 1.37E−30 | 1.91E−20 | 1.86E−29 |
| 28 | 2.20E−38 | 6.34E−16 | 0.00015078 | 3.00E−37 | 3.02E−15 | 2.23E−05 | 5.58E−26 |
| 29 | 2.16E−37 | 5.62E−21 | 1.99E−11 | 5.31E−38 | 4.07E−20 | 1.62E−14 | 1.79E−33 |
| 30 | 6.26E−38 | 1.31E−11 | 3.04E−05 | 7.55E−37 | 2.46E−19 | 5.99E−05 | 3.36E−19 |
| 31 | 1.70E−37 | 9.89E−20 | 4.00E−15 | 6.17E−33 | 1.59E−19 | 1.06E−07 | 5.60E−19 |
| 32 | 2.25E−37 | 3.62E−20 | 5.26E−16 | 3.31E−34 | 9.82E−23 | 4.38E−09 | 5.82E−22 |
| 33 | 2.44E−37 | 6.97E−16 | 4.33E−14 | 7.77E−37 | 9.30E−19 | 4.18E−07 | 1.39E−20 |
| 34 | 3.21E−37 | 1.12E−20 | 1.05E−11 | 3.70E−36 | 1.02E−20 | 3.75E−13 | 1.16E−24 |
| 35 | 4.66E−37 | 5.92E−20 | 3.00E−11 | 4.04E−32 | 8.17E−17 | 5.55E−10 | 2.22E−21 |
| 36 | 4.97E−37 | 9.12E−20 | 3.90E−11 | 7.27E−35 | 8.20E−18 | 2.95E−11 | 2.78E−26 |
| 37 | 5.87E−37 | 1.44E−15 | 3.36E−14 | 2.00E−36 | 3.55E−19 | 3.60E−07 | 3.09E−20 |
| 38 | 5.87E−37 | 1.44E−15 | 2.29E−13 | 2.00E−36 | 2.38E−18 | 1.01E−06 | 4.30E−21 |
| 39 | 7.97E−37 | 1.41E−20 | 4.11E−10 | 1.69E−34 | 6.22E−17 | 1.57E−12 | 9.61E−26 |
| 40 | 8.02E−36 | 1.02E−16 | 8.36E−13 | 8.02E−37 | 6.77E−18 | 2.61E−10 | 1.91E−19 |
| 41 | 1.05E−36 | 5.82E−22 | 6.09E−07 | 1.09E−33 | 3.93E−12 | 9.82E−13 | 1.13E−26 |
| 42 | 2.17E−18 | 1.05E−13 | 2.19E−05 | 7.67E−17 | 1.44E−05 | 1.58E−19 | 2.27E−36 |
| 43 | 2.04E−35 | 2.18E−16 | 4.05E−13 | 2.41E−36 | 4.89E−18 | 7.30E−10 | 2.59E−18 |
| 44 | 2.30E−34 | 4.36E−15 | 1.44E−11 | 8.19E−36 | 1.06E−16 | 9.26E−09 | 1.67E−18 |
| 45 | 8.67E−36 | 6.11E−17 | 3.23E−06 | 1.48E−32 | 1.25E−11 | 1.06E−09 | 8.79E−25 |
| 46 | 1.07E−35 | 6.07E−21 | 9.46E−09 | 3.15E−33 | 4.04E−15 | 6.09E−14 | 1.52E−24 |
| 47 | 2.15E−17 | 3.02E−13 | 2.37E−05 | 3.26E−16 | 2.56E−05 | 2.12E−19 | 1.73E−35 |
| 48 | 2.18E−35 | 2.69E−18 | 0.00016455 | 2.17E−32 | 2.13E−10 | 9.38E−10 | 2.81E−21 |
| 49 | 2.57E−35 | 5.01E−23 | 1.62E−06 | 3.27E−32 | 1.19E−11 | 4.78E−13 | 2.98E−26 |
| 50 | 2.89E−35 | 4.58E−19 | 5.87E−06 | 4.77E−32 | 5.92E−13 | 2.05E−11 | 8.30E−23 |

TABLE 2e
Data for Acinetobacter, particularly Acinetobacter baumanii (continued)

| No. | Locus.Tag | Gene.Symbol | GenBank.Accession | Chromosome |
|---|---|---|---|---|
| 1 | ABK1_1135 | | ADX02769 | |
| 2 | A60131_010100011170 | | ZP_06783174 | |
| 3 | A1S_0690 | | YP_001083736 | |
| 4 | ABBFA_001710 | | YP_002325613 | |
| 5 | ABBFA_001640 | | YP_002325543 | |
| 6 | AB57_1876 | | YP_002319238 | |
| 7 | AB57_2151 | | YP_002319507 | |
| 8 | ABBFA_001638 | | YP_002325541 | |
| 9 | AB57_2147 | | YP_002319503 | |
| 10 | AbauAB_010100009672 | | ZP_04661877 | |
| 11 | HMPREF0014_00181 | | ZP_05823167 | |
| 12 | HMPREF0014_02450 | | ZP_05825436 | |
| 13 | HMPREF0014_02452 | | ZP_05825438 | |
| 14 | AOLE_17735 | | YP_003733805 | |
| 15 | HMPREF0012_01352 | | ZP_06057184 | |
| 16 | HMPREF0014_02453 | | ZP_05825439 | |
| 17 | BDGL_001498 | ocd2 | ADY82084 | |
| 18 | HMPREF0014_02489 | | ZP_05825475 | |
| 19 | HMPREF0014_00004 | | ZP_05822990 | |
| 20 | ACIAD0370 | rbfA | YP_045140 | |
| 21 | HMPREF0014_02492 | | ZP_05825478 | |
| 22 | ABBFA_001637 | | YP_002325540 | |
| 23 | ABTW07_1894 | | ADX92323 | |
| 24 | ABBFA_000343 | | YP_002324276 | |
| 25 | AB57_2771 | | YP_002320110 | |
| 26 | AOLE_17690 | | YP_003733796 | |
| 27 | HMPREF0017_03097 | | ZP_06071176 | |
| 28 | A6013_010100003618 | | ZP_06795427 | |
| 29 | HMPREF0014_03338 | | ZP_05826324 | |
| 30 | A6014_010100015962 | | ZP_06788050 | |
| 31 | ACICU_00639 | filE | YP_001845298 | |
| 32 | HMPREF0013_02137 | | ZP_06692288 | |
| 33 | ABTW07_2865 | | ADX93289 | |
| 34 | HMPREF0016_03291 | | ZP_06064821 | |
| 35 | AB57_2131 | | YP_002319487 | |
| 36 | AB57_3624 | pilV | YP_002320921 | |
| 37 | ACICU_02614 | | YP_001847273 | |
| 38 | ABTW07_2863 | | ADX93287 | |
| 39 | AbauAB_010100013685 | | ZP_04662667 | |
| 40 | ABK1_2278 | | ADX03912 | |
| 41 | A6013_010100009420 | | ZP_06796568 | |
| 42 | HMPREF0010_03320 | | ZP_05829937 | |
| 43 | ACICU_01819 | | YP_001846478 | |
| 44 | HMP0015_1294 | | ZP_06727085 | |
| 45 | AB57_1430 | | YP_002318806 | |
| 46 | BDGL_000595 | | ADY81181 | |
| 47 | HMPREF0010_03319 | | ZP_05829936 | |
| 48 | ABBFA_001847 | | YP_002325750 | |
| 49 | HMPREF0014_00860 | | ZP_05823846 | |
| 50 | ABTW07_1891 | | ADX92320 | |

TABLE 2f
Data for Acinetobacter, particularly Acinetobacter baumanii (continued)

| No. | Start.Coord | End.Coord | Strand | Scaffold.ID |
|---|---|---|---|---|
| 1 | 1235814 | 1236425 | + | 650377914 |
| 2 | 64 | 768 | + | 647008664 |
| 3 | 821275 | 822057 | + | 640069344 |
| 4 | 1834753 | 1835667 | − | 643348583 |
| 5 | 1759838 | 1761280 | + | 643348583 |
| 6 | 1966831 | 1969149 | + | 643348581 |
| 7 | 2232790 | 2233716 | − | 643348581 |
| 8 | 1757930 | 1758571 | − | 643348583 |
| 9 | 2226774 | 2228099 | + | 643348581 |
| 10 | 63870 | 65015 | − | 645061599 |
| 11 | 195165 | 195419 | + | 647535325 |
| 12 | 27528 | 28301 | − | 647535345 |
| 13 | 31818 | 33023 | − | 647535345 |
| 14 | 3783514 | 3783789 | + | 648028017 |
| 15 | 1494626 | 1495630 | + | 646207323 |
| 16 | 33027 | 34796 | − | 647535345 |
| 17 | 1603798 | 1604811 | + | 650377930 |
| 18 | 74534 | 74893 | + | 647535345 |
| 19 | 1819 | 2325 | − | 647535325 |
| 20 | 363575 | 363976 | + | 637000277 |
| 21 | 76454 | 76681 | + | 647535345 |
| 22 | 1757594 | 1757890 | − | 643348583 |
| 23 | 2004366 | 2004983 | + | 650378014 |
| 24 | 370977 | 371978 | + | 643348583 |
| 25 | 2838051 | 2838620 | − | 643348581 |
| 26 | 3773081 | 3773467 | + | 648028017 |
| 27 | 716 | 1162 | + | 646207467 |
| 28 | 6958 | 7416 | − | 647008417 |
| 29 | 12541 | 13101 | − | 647535362 |
| 30 | 7588 | 7713 | + | 647009002 |
| 31 | 705957 | 707216 | + | 642555129 |
| 32 | 856694 | 857281 | − | 647536755 |
| 33 | 3040319 | 3041707 | − | 650378014 |
| 34 | 4553 | 4768 | − | 646207353 |
| 35 | 2213458 | 2214801 | − | 643348581 |
| 36 | 3730668 | 3731222 | − | 643348581 |
| 37 | 2775538 | 2776941 | + | 642555129 |
| 38 | 3038397 | 3039893 | − | 650378014 |
| 39 | 70983 | 71513 | − | 645061607 |
| 40 | 2403829 | 2405136 | + | 650377914 |
| 41 | 2603 | 3454 | + | 647008460 |
| 42 | 159696 | 159998 | + | 647535409 |
| 43 | 1941428 | 1942675 | + | 642555129 |
| 44 | 1088 | 2629 | − | 647018944 |
| 45 | 1505450 | 1506382 | + | 643348581 |
| 46 | 622112 | 622732 | + | 650377930 |
| 47 | 158794 | 159609 | + | 647535409 |
| 48 | 1980078 | 1981988 | − | 643348583 |
| 49 | 126664 | 127845 | + | 647535329 |
| 50 | 1999835 | 2001676 | + | 650378014 |

Examples 2 and 3: Enterobacter, particularly Enterobacter aerogenes and Enterobacter cloacae

The procedure was carried out as in Example 1, except that the following microorganisms were used:+

Bacterial Strains

The inventors selected 695 Enterobacter strains, particularly 298 for Enterobacter aerogenes and 397 for Enterobacter cloacae, from the microbiology strain collection at Siemens Healthcare Diagnostics (West Sacramento, Calif.) for susceptibility testing and whole genome sequencing.

From MetaRef, 5220 centroids of E. aerogenes were used as reference sequences for E. aerogenes, and 4734 centroids of E. cloacae were used as reference sequences for E. cloacae.

The results for Enterobacter aerogenes are shown in Tables 3 (corresponding to Table 1) and 4 (corresponding to Table 2), and the results for Enterobacter cloacae are shown in Tables 5 (corresponding to Table 1) and 6 (corresponding to Table 2).

TABLE 3a
Data for Enterobacter, particularly Enterobacter aerogenes

| | Scaffold.External. | | |
|---|---|---|---|
| No. | Accession | Scaffold.Name | best_pv |
| 1 | NC_015663 | Enterobacter aerogenes KCTC 2190 chromosome: NC_015663 | 1.01047730430126e−10 |
| 2 | NC_015663 | Enterobacter aerogenes KCTC 2190 chromosome: NC_015663 | 1.01047730430126e−10 |
| 3 | NC_015663 | Enterobacter aerogenes KCTC 2190 chromosome: NC_015663 | 2.21893843673589e−10 |
| 4 | NC_015663 | Enterobacter aerogenes KCTC 2190 chromosome: NC_015663 | 7.36056945497466e−08 |
| 5 | NC_015663 | Enterobacter aerogenes KCTC 2190 chromosome: NC_015663 | 9.81164607784395e−08 |
| 6 | NC_015663 | Enterobacter aerogenes KCTC 2190 chromosome: NC_015663 | 9.81164607784395e−08 |
| 7 | NC_015663 | Enterobacter aerogenes KCTC 2190 chromosome: NC_015663 | 3.92206261774063e−06 |
| 8 | NC_015663 | Enterobacter aerogenes KCTC 2190 chromosome: NC_015663 | 9.95623777570528e−06 |
| 9 | NC_015663 | Enterobacter aerogenes KCTC 2190 chromosome: NC_015663 | 0.000635187711382771 |
| 10 | NC_009778 | Enterobacter sakazakii ATCC BAA-894: NC_009778 | 0.000701852165838025 |
| 11 | NC_015663 | Enterobacter aerogenes KCTC 2190 chromosome: NC_015663 | 0.000717841336438945 |
| 12 | NZ_GG745509 | Klebsiella sp. 1_1_55 genomic scaffold supercont1.2: | 0.000881981942215321 |
| | | NZ_GG745509 | |
| 13 | NC_013850 | Klebsiella variicola At-22 chromosome: NC_013850 | 0.000881981942215321 |
| 14 | NC_015663 | Enterobacter aerogenes KCTC 2190 chromosome: NC_015663 | 0.000960532001115215 |
| 15 | NC_015663 | Enterobacter aerogenes KCTC 2190 chromosome: NC_015663 | 0.00108645793188005 |
| 16 | NZ_ADTJ01000031 | Escherichia coli MS 198-1 E_coli198-1-1.0_Cont45.1: | 0.00108724371491948 |
| | | NZ_ADTJ01000031 | |
| 17 | NC_015663 | Enterobacter aerogenes KCTC 2190 chromosome: NC_015663 | 0.0016524197402272 |
| 18 | NC_015663 | Enterobacter aerogenes KCTC 2190 chromosome: NC_015663 | 0.00184130315909336 |
| 19 | NC_015663 | Enterobacter aerogenes KCTC 2190 chromosome: NC_015663 | 0.00187260357604506 |
| 20 | NC_015663 | Enterobacter aerogenes KCTC 2190 chromosome: NC_015663 | 0.00191687062572367 |
| 21 | NZ_ACZD01000042 | Klebsiella pneumoniae subsp. rhinoscleromatis ATCC | 0.00208337132265191 |
| | | 13884 contig00042: NZ_ACZD01000042 | |
| 22 | NC_015663 | Enterobacter aerogenes KCTC 2190 chromosome: NC_015663 | 0.00208337132265191 |
| 23 | NZ_ADTJ01000031 | Escherichia coli MS 198-1 E_coli198-1-1.0_Cont45.1: | 0.00241352185546859 |
| | | NZ_ADTJ01000031 | |
| 24 | NZ_ADTJ01000031 | Escherichia coli MS 198-1 E_coli198-1-1.0_Cont45.1: | 0.00245969973695216 |
| | | NZ_ADTJ01000031 | |
| 25 | AM946981 | Escherichia coli BL21(DE3): AM946981 | 0.00327316953478549 |
| 26 | NC_015663 | Enterobacter aerogenes KCTC 2190 chromosome: NC_015663 | 0.00327864620009511 |
| 27 | NC_015663 | Enterobacter aerogenes KCTC 2190 chromosome: NC_015663 | 0.00334082507673483 |
| 28 | NC_015663 | Enterobacter aerogenes KCTC 2190 chromosome: NC_015663 | 0.00342044194519721 |
| 29 | NC_015663 | Enterobacter aerogenes KCTC 2190 chromosome: NC_015663 | 0.00349516820070151 |
| 30 | NC_015663 | Enterobacter aerogenes KCTC 2190 chromosome: NC_015663 | 0.00391582859992096 |
| 31 | NC_015663 | Enterobacter aerogenes KCTC 2190 chromosome: NC_015663 | 0.0039468230846432 |
| 32 | NC_015663 | Enterobacter aerogenes KCTC 2190 chromosome: NC_015663 | 0.0042274756954506 |
| 33 | NC_015663 | Enterobacter aerogenes KCTC 2190 chromosome: NC_015663 | 0.00458480687490629 |
| 34 | NC_015663 | Enterobacter aerogenes KCTC 2190 chromosome: NC_015663 | 0.00458480687490629 |
| 35 | NC_015663 | Enterobacter aerogenes KCTC 2190 chromosome: NC_015663 | 0.00458480687490629 |
| 36 | NC_012731 | Klebsiella pneumoniae NTUH-K2044: NC_012731 | 0.00458480687490629 |
| 37 | NC_015663 | Enterobacter aerogenes KCTC 2190 chromosome: NC_015663 | 0.00482300882771186 |
| 38 | NC_015663 | Enterobacter aerogenes KCTC 2190 chromosome: NC_015663 | 0.00495283743056749 |
| 39 | NC_015663 | Enterobacter aerogenes KCTC 2190 chromosome: NC_015663 | 0.00495283743056749 |
| 40 | NC_015663 | Enterobacter aerogenes KCTC 2190 chromosome: NC_015663 | 0.00503080982688659 |
| 41 | NC_015663 | Enterobacter aerogenes KCTC 2190 chromosome: NC_015663 | 0.00529748534762393 |
| 42 | NC_015663 | Enterobacter aerogenes KCTC 2190 chromosome: NC_015663 | 0.00585931541937669 |
| 43 | NC_015663 | Enterobacter aerogenes KCTC 2190 chromosome: NC_015663 | 0.00585931541937669 |
| 44 | NC_015663 | Enterobacter aerogenes KCTC 2190 chromosome: NC_015663 | 0.00585931541937669 |
| 45 | NC_015663 | Enterobacter aerogenes KCTC 2190 chromosome: NC_015663 | 0.00585931541937669 |
| 46 | NC_015663 | Enterobacter aerogenes KCTC 2190 chromosome: NC_015663 | 0.00648651344338336 |
| 47 | NC_015663 | Enterobacter aerogenes KCTC 2190 chromosome: NC_015663 | 0.00648651344338336 |
| 48 | NC_015663 | Enterobacter aerogenes KCTC 2190 chromosome: NC_015663 | 0.00648651344338336 |
| 49 | NZ_GG745510 | Klebsiella sp. 1_1_55 genomic scaffold supercont1.3: | 0.00659680763861341 |
| | | NZ_GG745510 | |
| 50 | NC_015663 | Enterobacter aerogenes KCTC 2190 chromosome: NC_015663 | 0.00667294667231188 |

TABLE 3b
Data for Enterobacter, particularly Enterobacter aerogenes (continued)

| No. | sign_phenos | sign_phenos_class | best_pheno | best_pheno_class |
|---|---|---|---|---|
| 1 | AZT; CAX; CAZ; CFT; CP; CRM; LVX; | aminoglycoside; fluoroquinolone; lactam; | CP | fluoroquinolone |
| | P/T; TO; T/S | other | | |
| 2 | AZT; CAX; CAZ; CFT; CP; CRM; | aminoglycoside; fluoroquinolone; lactam; | CP | fluoroquinolone |
| | LVX; P/T; TO; T/S | other | | |
| 3 | AZT; CAX; CAZ; CFT; CP; CRM; | aminoglycoside; fluoroquinolone; lactam; | CP | fluoroquinolone |
| | LVX; P/T; TO; T/S | other | | |
| 4 | CP; LVX; TO; T/S | aminoglycoside; fluoroquinolone; other | CP | fluoroquinolone |
| 5 | AZT; CAX; CAZ; CFT; CP; CRM; LVX; P/T; T/S | fluoroquinolone; lactam; other | CP | fluoroquinolone |
| 6 | AZT; CP; LVX; P/T; T/S | fluoroquinolone; lactam; other | CP | fluoroquinolone |
| 7 | CP; LVX; T/S | fluoroquinolone; other | CP | fluoroquinolone |
| 8 | CP; LVX; T/S | fluoroquinolone; other | CP | fluoroquinolone |
| 9 | AZT; CAX; CAZ; CFT; P/T | lactam | CFT | lactam |
| 10 | AZT; CAX; CAZ; CFT; CRM; P/T | lactam | AZT | lactam |
| 11 | CAX; CFT; CRM | lactam | CRM | lactam |
| 12 | AZT; CAX; CAZ; CFT; P/T | lactam | CAX | lactam |
| 13 | AZT; CAX; CAZ; CFT; P/T | lactam | CAX | lactam |
| 14 | TO | aminoglycoside | TO | aminoglycoside |
| 15 | AZT; CAX; CAZ; CFT; CRM; P/T | lactam | CAX | lactam |
| 16 | CP; LVX | fluoroquinolone | CP | fluoroquinolone |
| 17 | AZT; CAX; CAZ; CFT; CP; P/T | fluoroquinolone; lactam | P/T | lactam |
| 18 | AZT; CAX; CAZ; CFT; CP; P/T | fluoroquinolone; lactam | CAX | lactam |
| 19 | AZT; CAX; CAZ; CFT; CRM | lactam | CAX | lactam |
| 20 | CAX; CAZ; CFT | lactam | CAX | lactam |
| 21 | CAX; CAZ; CFT; CRM; P/T | lactam | CAX | lactam |
| 22 | CAX; CAZ; CFT; CRM; P/T | lactam | CAX | lactam |
| 23 | TO | aminoglycoside | TO | aminoglycoside |
| 24 | TO | aminoglycoside | TO | aminoglycoside |
| 25 | AZT; CAX; CAZ; CFT | lactam | CAX | lactam |
| 26 | CP | fluoroquinolone | CP | fluoroquinolone |
| 27 | AZT; CAX; CAZ; CFT; CRM; P/T | lactam | CAX | lactam |
| 28 | CAX; CAZ; CFT; CRM; P/T | lactam | CAX | lactam |
| 29 | AZT; CAX; CAZ; CFT | lactam | CAX | lactam |
| 30 | AZT; CAX; CAZ; CFT; P/T | lactam | AZT | lactam |
| 31 | AZT; CAX; CFT | lactam | CAX | lactam |
| 32 | AZT; CAX | lactam | CAX | lactam |
| 33 | CAX; CFT | lactam | CAX | lactam |
| 34 | CAX; CFT | lactam | CAX | lactam |
| 35 | CAX; CP | fluoroquinolone; lactam | CP | fluoroquinolone |
| 36 | CAX; CFT | lactam | CAX | lactam |
| 37 | CAX; CFT; CRM; P/T | lactam | CRM | lactam |
| 38 | AZT; CAX; CFT | lactam | CFT | lactam |
| 39 | AZT; CAX; CFT | lactam | CFT | lactam |
| 40 | CAX | lactam | CAX | lactam |
| 41 | CAX | lactam | CAX | lactam |
| 42 | AZT; CAX | lactam | AZT | lactam |
| 43 | CAX | lactam | CAX | lactam |
| 44 | AZT; CAX | lactam | AZT | lactam |
| 45 | AZT; CAX; CFT | lactam | CAX | lactam |
| 46 | AZT; CAX | lactam | CAX | lactam |
| 47 | AZT; CAX | lactam | CAX | lactam |
| 48 | AZT; CAX | lactam | CAX | lactam |
| 49 | CAX | lactam | CAX | lactam |
| 50 | CAX | lactam | CAX | lactam |

TABLE 3c
Data for Enterobacter, particularly Enterobacter aerogenes (continued)

| No. | num_aminoglycoside | num_fluoroquinolone | num_lactam | num_other |
|---|---|---|---|---|
| 1 | 1 | 2 | 6 | 1 |
| 2 | 1 | 2 | 6 | 1 |
| 3 | 1 | 2 | 6 | 1 |
| 4 | 1 | 2 | 0 | 1 |
| 5 | 0 | 2 | 6 | 1 |
| 6 | 0 | 2 | 2 | 1 |
| 7 | 0 | 2 | 0 | 1 |
| 8 | 0 | 2 | 0 | 1 |
| 9 | 0 | 0 | 5 | 0 |
| 10 | 0 | 0 | 6 | 0 |
| 11 | 0 | 0 | 3 | 0 |
| 12 | 0 | 0 | 5 | 0 |
| 13 | 0 | 0 | 5 | 0 |
| 14 | 1 | 0 | 0 | 0 |
| 15 | 0 | 0 | 6 | 0 |
| 16 | 0 | 2 | 0 | 0 |
| 17 | 0 | 1 | 5 | 0 |
| 18 | 0 | 1 | 5 | 0 |
| 19 | 0 | 0 | 5 | 0 |
| 20 | 0 | 0 | 3 | 0 |
| 21 | 0 | 0 | 5 | 0 |
| 22 | 0 | 0 | 5 | 0 |
| 23 | 1 | 0 | 0 | 0 |
| 24 | 1 | 0 | 0 | 0 |
| 25 | 0 | 0 | 4 | 0 |
| 26 | 0 | 1 | 0 | 0 |
| 27 | 0 | 0 | 6 | 0 |
| 28 | 0 | 0 | 5 | 0 |
| 29 | 0 | 0 | 4 | 0 |
| 30 | 0 | 0 | 5 | 0 |
| 31 | 0 | 0 | 3 | 0 |
| 32 | 0 | 0 | 2 | 0 |
| 33 | 0 | 0 | 2 | 0 |
| 34 | 0 | 0 | 2 | 0 |
| 35 | 0 | 1 | 1 | 0 |
| 36 | 0 | 0 | 2 | 0 |
| 37 | 0 | 0 | 4 | 0 |
| 38 | 0 | 0 | 3 | 0 |
| 39 | 0 | 0 | 3 | 0 |
| 40 | 0 | 0 | 1 | 0 |
| 41 | 0 | 0 | 1 | 0 |
| 42 | 0 | 0 | 2 | 0 |
| 43 | 0 | 0 | 1 | 0 |
| 44 | 0 | 0 | 2 | 0 |
| 45 | 0 | 0 | 3 | 0 |
| 46 | 0 | 0 | 2 | 0 |
| 47 | 0 | 0 | 2 | 0 |
| 48 | 0 | 0 | 2 | 0 |
| 49 | 0 | 0 | 1 | 0 |
| 50 | 0 | 0 | 1 | 0 |

TABLE 3d
Data for Enterobacter, particularly Enterobacter aerogenes (continued)

| No. | AZT_pv_adj | CAX_pv_adj | CAZ_pv_adj | CFT_pv_adj | CP_pv_adj |
|---|---|---|---|---|---|
| 1 | 8.22E−04 | 2.45E−04 | 3.83E−04 | 4.31E−04 | 1.01E−10 |
| 2 | 1.32E−03 | 4.48E−04 | 7.06E−04 | 7.76E−04 | 1.01E−10 |
| 3 | 3.27E−03 | 1.99E−03 | 4.43E−03 | 3.01E−03 | 2.22E−10 |
| 4 | 2.06E−02 | 1.86E−02 | 2.29E−02 | 2.41E−02 | 7.36E−08 |
| 5 | 4.29E−03 | 2.32E−04 | 6.91E−04 | 7.06E−04 | 9.81E−08 |
| 6 | 8.86E−03 | 1.82E−02 | 1.77E−02 | 1.84E−02 | 9.81E−08 |
| 7 | 5.47E−01 | 1.61E−02 | 4.75E−02 | 4.90E−02 | 3.92E−06 |
| 8 | 6.96E−01 | 3.29E−02 | 9.63E−02 | 9.80E−02 | 9.96E−06 |
| 9 | 4.82E−03 | 9.01E−04 | 1.84E−03 | 6.35E−04 | 3.94E−02 |
| 10 | 7.02E−04 | 3.95E−03 | 6.47E−03 | 6.18E−03 | 2.19E−02 |
| 11 | 1.74E−02 | 4.82E−03 | 1.41E−02 | 4.93E−03 | 4.78E−01 |
| 12 | 5.48E−03 | 8.82E−04 | 6.43E−03 | 2.81E−03 | 2.06E−01 |
| 13 | 5.48E−03 | 8.82E−04 | 6.43E−03 | 2.81E−03 | 2.06E−01 |
| 14 | 6.13E−01 | 3.30E−01 | 3.24E−01 | 4.36E−01 | 1.68E−02 |
| 15 | 3.27E−03 | 1.09E−03 | 2.08E−03 | 3.16E−03 | 1.86E−02 |
| 16 | 1.54E−01 | 7.15E−02 | 8.90E−02 | 1.12E−01 | 1.09E−03 |
| 17 | 3.27E−03 | 5.44E−03 | 6.66E−03 | 5.44E−03 | 6.21E−03 |
| 18 | 5.86E−03 | 1.84E−03 | 2.44E−03 | 3.43E−03 | 5.30E−03 |
| 19 | 2.04E−03 | 1.87E−03 | 6.34E−03 | 2.86E−03 | 4.83E−01 |
| 20 | 2.06E−02 | 1.92E−03 | 6.18E−03 | 3.56E−03 | 1.74E−01 |
| 21 | 2.06E−02 | 2.08E−03 | 6.49E−03 | 5.34E−03 | 4.77E−01 |
| 22 | 2.06E−02 | 2.08E−03 | 6.49E−03 | 5.34E−03 | 4.77E−01 |
| 23 | 3.45E−01 | 2.11E−01 | 2.09E−01 | 2.56E−01 | 4.61E−02 |
| 24 | 2.34E−01 | 1.31E−01 | 1.03E−01 | 1.64E−01 | 1.86E−02 |
| 25 | 3.92E−03 | 3.27E−03 | 9.44E−03 | 4.58E−03 | 6.15E−01 |
| 26 | 1.66E−02 | 3.39E−02 | 3.18E−02 | 4.22E−02 | 3.28E−03 |
| 27 | 3.46E−03 | 3.34E−03 | 5.74E−03 | 5.48E−03 | 2.58E−01 |
| 28 | 1.26E−02 | 3.42E−03 | 6.74E−03 | 5.59E−03 | 6.42E−02 |
| 29 | 5.74E−03 | 3.50E−03 | 9.56E−03 | 4.82E−03 | 4.25E−01 |
| 30 | 3.92E−03 | 5.86E−03 | 9.75E−03 | 7.85E−03 | 4.70E−01 |
| 31 | 5.99E−03 | 3.95E−03 | 1.08E−02 | 5.34E−03 | 5.16E−01 |
| 32 | 8.78E−03 | 4.23E−03 | 2.75E−02 | 1.06E−02 | 3.44E−01 |
| 33 | 2.32E−02 | 4.58E−03 | 2.06E−02 | 9.82E−03 | 6.54E−01 |
| 34 | 2.32E−02 | 4.58E−03 | 2.06E−02 | 9.82E−03 | 6.54E−01 |
| 35 | 2.01E−02 | 5.71E−03 | 1.85E−02 | 1.46E−02 | 4.58E−03 |
| 36 | 2.32E−02 | 4.58E−03 | 2.06E−02 | 9.82E−03 | 6.54E−01 |
| 37 | 3.09E−02 | 6.66E−03 | 2.19E−02 | 9.73E−03 | 4.29E−01 |
| 38 | 9.88E−03 | 8.78E−03 | 1.16E−02 | 4.95E−03 | 1.65E−01 |
| 39 | 9.88E−03 | 8.78E−03 | 1.16E−02 | 4.95E−03 | 1.65E−01 |
| 40 | 3.62E−02 | 5.03E−03 | 2.31E−02 | 1.38E−02 | 2.47E−01 |
| 41 | 1.54E−02 | 5.30E−03 | 1.43E−02 | 1.12E−02 | 6.43E−02 |
| 42 | 5.86E−03 | 9.44E−03 | 1.23E−02 | 2.06E−02 | 7.63E−02 |
| 43 | 1.15E−02 | 5.86E−03 | 1.39E−02 | 1.38E−02 | 2.62E−01 |
| 44 | 5.86E−03 | 9.44E−03 | 1.23E−02 | 2.06E−02 | 7.63E−02 |
| 45 | 9.73E−03 | 5.86E−03 | 1.64E−02 | 6.17E−03 | 4.25E−01 |
| 46 | 7.89E−03 | 6.49E−03 | 3.09E−02 | 1.64E−02 | 2.98E−01 |
| 47 | 7.89E−03 | 6.49E−03 | 3.09E−02 | 1.64E−02 | 2.98E−01 |
| 48 | 7.89E−03 | 6.49E−03 | 3.09E−02 | 1.64E−02 | 2.98E−01 |
| 49 | 1.20E−02 | 6.60E−03 | 1.12E−02 | 1.82E−02 | 2.16E−01 |
| 50 | 1.38E−02 | 6.67E−03 | 2.06E−02 | 1.64E−02 | 3.23E−01 |

TABLE 3e
Data for Enterobacter, particularly Enterobacter aerogenes (continued)

| No. | CRM_pv_adj | LVX_pv_adj | P/T_pv_adj | TO_pv_adj | T/S_pv_adj |
|---|---|---|---|---|---|
| 1 | 4.55E−04 | 5.22E−07 | 2.23E−04 | 2.03E−04 | 1.88E−06 |
| 2 | 8.93E−04 | 7.25E−07 | 2.87E−04 | 2.45E−04 | 2.53E−06 |
| 3 | 1.47E−03 | 8.82E−07 | 4.54E−03 | 2.55E−04 | 3.69E−06 |
| 4 | 1.86E−02 | 5.08E−05 | 2.73E−02 | 3.92E−03 | 3.62E−05 |
| 5 | 9.61E−04 | 7.96E−07 | 9.64E−04 | 1.26E−02 | 1.69E−03 |
| 6 | 1.46E−02 | 1.31E−04 | 8.95E−03 | 2.06E−02 | 8.55E−03 |
| 7 | 4.85E−02 | 6.35E−04 | 2.98E−01 | 9.28E−02 | 9.01E−04 |
| 8 | 1.03E−01 | 9.82E−04 | 4.28E−01 | 1.13E−01 | 1.44E−03 |
| 9 | 1.08E−02 | 2.67E−01 | 3.56E−03 | 5.09E−01 | 2.87E−01 |
| 10 | 1.66E−03 | 1.39E−01 | 5.16E−03 | 8.93E−02 | 1.08E−01 |
| 11 | 7.18E−04 | 5.91E−01 | 1.76E−02 | 7.39E−01 | 9.14E−01 |
| 12 | 7.03E−02 | 6.77E−01 | 6.85E−03 | 7.65E−01 | 9.68E−01 |
| 13 | 7.03E−02 | 6.77E−01 | 6.85E−03 | 7.65E−01 | 9.68E−01 |
| 14 | 8.23E−01 | 8.17E−02 | 2.13E−01 | 9.61E−04 | 2.24E−02 |
| 15 | 4.37E−03 | 2.22E−01 | 9.03E−03 | 2.02E−01 | 2.91E−01 |
| 16 | 4.57E−02 | 7.49E−03 | 2.26E−01 | 4.29E−01 | 4.38E−02 |
| 17 | 3.26E−02 | 1.15E−01 | 1.65E−03 | 1.93E−02 | 4.43E−02 |
| 18 | 1.64E−02 | 1.66E−01 | 9.73E−03 | 4.25E−01 | 6.31E−01 |
| 19 | 3.50E−03 | 6.96E−01 | 3.47E−02 | 1 | 1 |
| 20 | 1.20E−02 | 4.90E−01 | 1.60E−02 | 1 | 7.91E−01 |
| 21 | 2.40E−03 | 9.70E−01 | 9.40E−03 | 4.93E−01 | 6.15E−01 |
| 22 | 2.40E−03 | 9.70E−01 | 9.40E−03 | 4.93E−01 | 6.15E−01 |
| 23 | 5.50E−01 | 2.07E−01 | 1.23E−01 | 2.41E−03 | 4.88E−02 |
| 24 | 5.46E−01 | 9.58E−02 | 1.16E−01 | 2.46E−03 | 7.04E−02 |
| 25 | 1.40E−02 | 8.28E−01 | 4.99E−02 | 9.45E−01 | 8.28E−01 |
| 26 | 4.65E−02 | 5.56E−02 | 1.64E−02 | 4.80E−02 | 1.26E−02 |
| 27 | 9.73E−03 | 5.14E−01 | 9.59E−03 | 7.93E−01 | 8.06E−01 |
| 28 | 5.86E−03 | 3.56E−01 | 4.54E−03 | 3.41E−01 | 5.47E−01 |
| 29 | 2.85E−02 | 7.23E−01 | 6.42E−02 | 8.28E−01 | 6.13E−01 |
| 30 | 1.82E−02 | 6.97E−01 | 5.99E−03 | 6.87E−01 | 9.71E−01 |
| 31 | 1.59E−02 | 8.28E−01 | 7.52E−02 | 8.28E−01 | 7.23E−01 |
| 32 | 1.61E−02 | 6.13E−01 | 9.20E−02 | 9.51E−01 | 8.47E−01 |
| 33 | 8.16E−02 | 9.71E−01 | 3.93E−02 | 3.34E−01 | 7.11E−01 |
| 34 | 8.16E−02 | 9.71E−01 | 3.93E−02 | 3.34E−01 | 7.11E−01 |
| 35 | 5.70E−02 | 3.62E−02 | 1.61E−02 | 1.47E−01 | 4.48E−02 |
| 36 | 8.16E−02 | 9.71E−01 | 3.93E−02 | 3.34E−01 | 7.11E−01 |
| 37 | 4.82E−03 | 3.59E−01 | 5.54E−03 | 7.80E−01 | 2.77E−01 |
| 38 | 1.33E−02 | 4.16E−01 | 4.96E−02 | 7.39E−01 | 5.42E−01 |
| 39 | 1.33E−02 | 4.16E−01 | 4.96E−02 | 7.39E−01 | 5.42E−01 |
| 40 | 1.95E−02 | 5.02E−01 | 5.98E−02 | 2.35E−01 | 2.07E−01 |
| 41 | 4.20E−02 | 2.18E−01 | 1.19E−02 | 2.02E−01 | 4.53E−01 |
| 42 | 7.24E−02 | 4.76E−01 | 1.76E−02 | 9.62E−01 | 7.80E−01 |
| 43 | 6.56E−02 | 5.59E−01 | 9.19E−02 | 8.66E−01 | 1 |
| 44 | 7.24E−02 | 4.76E−01 | 1.76E−02 | 9.62E−01 | 7.80E−01 |
| 45 | 1.76E−02 | 7.23E−01 | 1.10E−01 | 8.28E−01 | 7.24E−01 |
| 46 | 1.84E−02 | 6.37E−01 | 9.12E−02 | 7.39E−01 | 1 |
| 47 | 1.84E−02 | 6.37E−01 | 9.12E−02 | 7.39E−01 | 1 |
| 48 | 1.84E−02 | 6.37E−01 | 9.12E−02 | 7.39E−01 | 1 |
| 49 | 7.35E−02 | 7.11E−01 | 1.15E−02 | 5.12E−01 | 3.68E−01 |
| 50 | 1.28E−01 | 6.61E−01 | 1.11E−01 | 9.60E−01 | 1 |

TABLE 3f
Data for Enterobacter, particularly Enterobacter aerogenes (continued)

| No. | Locus.Tag | Gene.Symbol | GenBank.Accession | Chromosome |
|---|---|---|---|---|
| 1 | EAE_18905 | | YP_004593967 | |
| 2 | EAE_18910 | | YP_004593968 | |
| 3 | EAE_20750 | | YP_004594334 | |
| 4 | EAE_20680 | | YP_004594322 | |
| 5 | EAE_10325 | | YP_004592260 | |
| 6 | EAE_15945 | | YP_004593379 | |
| 7 | EAE_24270 | | YP_004595026 | |
| 8 | EAE_19175 | | YP_004594021 | |
| 9 | EAE_17360 | | YP_004593662 | |
| 10 | ESA_03685 | | YP_001439725 | |
| 11 | EAE_20530 | tynA | YP_004594292 | |
| 12 | HMPREF0485_02019 | | ZP_06549619 | |
| 13 | Kvar_3843 | | YP_003440755 | |
| 14 | EAE_19000 | | YP_004593986 | |
| 15 | EAE_21240 | | YP_004594432 | |
| 16 | HMPREF9552_00257 | | ZP_07114469 | |
| 17 | EAE_11550 | | YP_004592503 | |
| 18 | EAE_21235 | | YP_004594431 | |
| 19 | EAE_24605 | flk | YP_004595093 | |
| 20 | EAE_17355 | | YP_004593661 | |
| 21 | HMPREF0484_0684 | | ZP_06013668 | |
| 22 | EAE_06855 | | YP_004591574 | |
| 23 | HMPREF9552_00255 | | ZP_07114467 | |
| 24 | HMPREF9552_00247 | | ZP_07114459 | |
| 25 | B21_02377 | ybl112 | CAQ32894 | |
| 26 | EAE_00560 | | YP_004590329 | |
| 27 | EAE_02625 | aphA | YP_004590739 | |
| 28 | EAE_10300 | | YP_004592255 | |
| 29 | EAE_23410 | | YP_004594860 | |
| 30 | EAE_02640 | | YP_004590742 | |
| 31 | EAE_23150 | | YP_004594808 | |
| 32 | EAE_23330 | | YP_004594844 | |
| 33 | EAE_06345 | | YP_004591472 | |
| 34 | EAE_06340 | | YP_004591471 | |
| 35 | EAE_23435 | | YP_004594865 | |
| 36 | KP1_5319 | wabN | YP_002921814 | |
| 37 | EAE_22395 | | YP_004594657 | |
| 38 | EAE_06770 | | YP_004591557 | |
| 39 | EAE_06775 | | YP_004591558 | |
| 40 | EAE_06430 | ligB | YP_004591489 | |
| 41 | EAE_15075 | | YP_004593207 | |
| 42 | EAE_03420 | | YP_004590898 | |
| 43 | EAE_23445 | | YP_004594867 | |
| 44 | EAE_03430 | | YP_004590900 | |
| 45 | EAE_23415 | | YP_004594861 | |
| 46 | EAE_06330 | | YP_004591469 | |
| 47 | EAE_06320 | | YP_004591467 | |
| 48 | EAE_06325 | | YP_004591468 | |
| 49 | HMPREF0485_03181 | | ZP_06550780 | |
| 50 | EAE_23450 | | YP_004594868 | |

TABLE 3g
Data for Enterobacter, particularly Enterobacter aerogenes (continued)

| No. | Start.Coord | End.Coord | Strand | Scaffold.ID |
|---|---|---|---|---|
| 1 | 4057377 | 4057994 | − | 650716122 |
| 2 | 4057994 | 4059313 | − | 650716122 |
| 3 | 4444576 | 4447206 | − | 650716122 |
| 4 | 4432502 | 4433842 | − | 650716122 |
| 5 | 2202535 | 2203893 | − | 650716122 |
| 6 | 3439556 | 3442066 | + | 650716122 |
| 7 | 5174177 | 5175436 | + | 650716122 |
| 8 | 4113374 | 4114306 | − | 650716122 |
| 9 | 3748049 | 3748279 | + | 650716122 |
| 10 | 3623778 | 3624146 | + | 640753067 |
| 11 | 4396852 | 4399119 | + | 650716122 |
| 12 | 347124 | 347486 | + | 647536390 |
| 13 | 4046094 | 4046396 | − | 646312028 |
| 14 | 4078519 | 4079427 | + | 650716122 |
| 15 | 4545796 | 4546983 | − | 650716122 |
| 16 | 14233 | 15036 | + | 648286894 |
| 17 | 2477524 | 2479728 | + | 650716122 |
| 18 | 4544187 | 4545296 | + | 650716122 |
| 19 | 5241493 | 5242410 | + | 650716122 |
| 20 | 3747138 | 3747791 | + | 650716122 |
| 21 | 5606 | 5998 | + | 647009789 |
| 22 | 1421376 | 1421699 | + | 650716122 |
| 23 | 12357 | 13376 | + | 648286894 |
| 24 | 7786 | 8685 | − | 648286894 |
| 25 | 2517630 | 2518469 | + | 646862300 |
| 26 | 114571 | 115482 | + | 650716122 |
| 27 | 557964 | 558680 | − | 650716122 |
| 28 | 2197515 | 2198873 | + | 650716122 |
| 29 | 4974131 | 4974946 | − | 650716122 |
| 30 | 560166 | 561344 | − | 650716122 |
| 31 | 4919559 | 4920611 | + | 650716122 |
| 32 | 4955309 | 4956457 | + | 650716122 |
| 33 | 1317545 | 1318672 | + | 650716122 |
| 34 | 1316472 | 1317548 | + | 650716122 |
| 35 | 4979540 | 4980544 | + | 650716122 |
| 36 | 5064271 | 5065293 | − | 646564530 |
| 37 | 4777378 | 4777638 | − | 650716122 |
| 38 | 1404935 | 1406026 | − | 650716122 |
| 39 | 1406073 | 1406483 | + | 650716122 |
| 40 | 1331758 | 1333431 | − | 650716122 |
| 41 | 3235642 | 3239709 | + | 650716122 |
| 42 | 718097 | 718942 | − | 650716122 |
| 43 | 4982582 | 4983136 | − | 650716122 |
| 44 | 720009 | 720686 | + | 650716122 |
| 45 | 4975309 | 4976445 | − | 650716122 |
| 46 | 1314302 | 1315078 | − | 650716122 |
| 47 | 1312221 | 1313129 | + | 650716122 |
| 48 | 1313194 | 1314276 | + | 650716122 |
| 49 | 538177 | 539649 | + | 647536391 |
| 50 | 4983148 | 4984038 | − | 650716122 |

TABLE 4a
Data for Enterobacter, particularly Enterobacter aerogenes

| | Scaffold.External. | | |
|---|---|---|---|
| No. | Accession | Scaffold.Name | best_pv |
| 1 | NC_015663 | Enterobacter aerogenes KCTC 2190 chromosome: NC_015663 | 1.01047730430126e−10 |
| 2 | NC_015663 | Enterobacter aerogenes KCTC 2190 chromosome: NC_015663 | 1.01047730430126e−10 |
| 3 | NC_015663 | Enterobacter aerogenes KCTC 2190 chromosome: NC_015663 | 2.21893843673589e−10 |
| 4 | NC_015663 | Enterobacter aerogenes KCTC 2190 chromosome: NC_015663 | 7.36056945497466e−08 |
| 5 | NC_015663 | Enterobacter aerogenes KCTC 2190 chromosome: NC_015663 | 9.81164607784395e−08 |
| 6 | NC_015663 | Enterobacter aerogenes KCTC 2190 chromosome: NC_015663 | 9.81164607784395e−08 |
| 7 | NC_015663 | Enterobacter aerogenes KCTC 2190 chromosome: NC_015663 | 3.92206261774063e−06 |
| 8 | NC_015663 | Enterobacter aerogenes KCTC 2190 chromosome: NC_015663 | 9.95623777570528e−06 |
| 9 | NC_009778 | Enterobacter sakazakii ATCC BAA-894: NC_009778 | 0.000701852165838025 |
| 10 | NC_015663 | Enterobacter aerogenes KCTC 2190 chromosome: NC_015663 | 0.000960532001115215 |
| 11 | NC_015663 | Enterobacter aerogenes KCTC 2190 chromosome: NC_015663 | 0.00108645793188005 |
| 12 | NZ_ADTJ01000031 | Escherichia coli MS 198-1 E_coli198-1-1.0_Cont45.1: | 0.00108724371491948 |
| | | NZ_ADTJ01000031 | |
| 13 | NZ_ADTJ01000031 | Escherichia coli MS 198-1 E_coli198-1-1.0_Cont45.1: | 0.00241352185546859 |
| | | NZ_ADTJ01000031 | |
| 14 | NZ_ADTJ01000031 | Escherichia coli MS 198-1 E_coli198-1-1.0_Cont45.1: | 0.00245969973695216 |
| | | NZ_ADTJ01000031 | |
| 15 | NC_015663 | Enterobacter aerogenes KCTC 2190 chromosome: NC_015663 | 0.00334082507673483 |
| 16 | NC_015663 | Enterobacter aerogenes KCTC 2190 chromosome: NC_015663 | 0.00795925223735345 |

TABLE 4b
Data for Enterobacter, particularly Enterobacter aerogenes (continued)

| No. | sign_phenos | sign_phenos_class | best_pheno | best_pheno_class |
|---|---|---|---|---|
| 1 | AZT; CAX; CAZ; CFT; CP; CRM; LVX; P/T; TO; T/S | aminoglycoside; fluoroquinolone; lactam; | CP | fluoroquinolone |
| | | other | | |
| 2 | AZT; CAX; CAZ; CFT; CP; CRM; LVX; P/T; TO; T/S | aminoglycoside; fluoroquinolone; lactam; | CP | fluoroquinolone |
| | | other | | |
| 3 | AZT; CAX; CAZ; CFT; CP; CRM; LVX; P/T; TO; T/S | aminoglycoside; fluoroquinolone; lactam; | CP | fluoroquinolone |
| | | other | | |
| 4 | CP; LVX; TO; T/S | aminoglycoside; fluoroquinolone; other | CP | fluoroquinolone |
| 5 | AZT; CAX; CAZ; CFT; CP; CRM; LVX; P/T; T/S | fluoroquinolone; lactam; other | CP | fluoroquinolone |
| 6 | AZT; CP; LVX; P/T; T/S | fluoroquinolone; lactam; other | CP | fluoroquinolone |
| 7 | CP; LVX; T/S | fluoroquinolone; other | CP | fluoroquinolone |
| 8 | CP; LVX; T/S | fluoroquinolone; other | CP | fluoroquinolone |
| 9 | AZT; CAX; CAZ; CFT; CRM; P/T | lactam | AZT | lactam |
| 10 | TO | aminoglycoside | TO | aminoglycoside |
| 11 | AZT; CAX; CAZ; CFT; CRM; P/T | lactam | CAX | lactam |
| 12 | CP; LVX | fluoroquinolone | CP | fluoroquinolone |
| 13 | TO | aminoglycoside | TO | aminoglycoside |
| 14 | TO | aminoglycoside | TO | aminoglycoside |
| 15 | AZT; CAX; CAZ; CFT; CRM; P/T | lactam | CAX | lactam |
| 16 | TO | aminoglycoside | TO | aminoglycoside |

TABLE 4c
Data for Enterobacter, particularly Enterobacter aerogenes (continued)

| No. | num_aminoglycoside | num_fluoroquinolone | num_lactam | num_other |
|---|---|---|---|---|
| 1 | 1 | 2 | 6 | 1 |
| 2 | 1 | 2 | 6 | 1 |
| 3 | 1 | 2 | 6 | 1 |
| 4 | 1 | 2 | 0 | 1 |
| 5 | 0 | 2 | 6 | 1 |
| 6 | 0 | 2 | 2 | 1 |
| 7 | 0 | 2 | 0 | 1 |
| 8 | 0 | 2 | 0 | 1 |
| 9 | 0 | 0 | 6 | 0 |
| 10 | 1 | 0 | 0 | 0 |
| 11 | 0 | 0 | 6 | 0 |
| 12 | 0 | 2 | 0 | 0 |
| 13 | 1 | 0 | 0 | 0 |
| 14 | 1 | 0 | 0 | 0 |
| 15 | 0 | 0 | 6 | 0 |
| 16 | 1 | 0 | 0 | 0 |

TABLE 4d
Data for Enterobacter, particularly Enterobacter aerogenes (continued)

| No. | AZT_pv_adj | CAX_pv_adj | CAZ_pv_adj | CFT_pv_adj | CP_pv_adj |
|---|---|---|---|---|---|
| 1 | 8.22E−04 | 2.45E−04 | 3.83E−04 | 4.31E−04 | 1.01E−10 |
| 2 | 1.32E−03 | 4.48E−04 | 7.06E−04 | 7.76E−04 | 1.01E−10 |
| 3 | 3.27E−03 | 1.99E−03 | 4.43E−03 | 3.01E−03 | 2.22E−10 |
| 4 | 2.06E−02 | 1.86E−02 | 2.29E−02 | 2.41E−02 | 7.36E−08 |
| 5 | 4.29E−03 | 2.32E−04 | 6.91E−04 | 7.06E−04 | 9.81E−08 |
| 6 | 8.86E−03 | 1.82E−02 | 1.77E−02 | 1.84E−02 | 9.81E−08 |
| 7 | 5.47E−01 | 1.61E−02 | 4.75E−02 | 4.90E−02 | 3.92E−06 |
| 8 | 6.96E−01 | 3.29E−02 | 9.63E−02 | 9.80E−02 | 9.96E−06 |
| 9 | 7.02E−04 | 3.95E−03 | 6.47E−03 | 6.18E−03 | 2.19E−02 |
| 10 | 6.13E−01 | 3.30E−01 | 3.24E−01 | 4.36E−01 | 1.68E−02 |
| 11 | 3.27E−03 | 1.09E−03 | 2.08E−03 | 3.16E−03 | 1.86E−02 |
| 12 | 1.54E−01 | 7.15E−02 | 8.90E−02 | 1.12E−01 | 1.09E−03 |
| 13 | 3.45E−01 | 2.11E−01 | 2.09E−01 | 2.56E−01 | 4.61E−02 |
| 14 | 2.34E−01 | 1.31E−01 | 1.03E−01 | 1.64E−01 | 1.86E−02 |
| 15 | 3.46E−03 | 3.34E−03 | 5.74E−03 | 5.48E−03 | 2.58E−01 |
| 16 | 3.30E−01 | 2.07E−01 | 1.34E−01 | 1.68E−01 | 8.94E−01 |

TABLE 4e
Data for Enterobacter, particularly Enterobacter aerogenes (continued)

| No. | CRM_pv_adj | LVX_pv_adj | P/T_pv_adj | TO_pv_adj | T/S_pv_adj |
|---|---|---|---|---|---|
| 1 | 4.55E−04 | 5.22E−07 | 2.23E−04 | 2.03E−04 | 1.88E−06 |
| 2 | 8.93E−04 | 7.25E−07 | 2.87E−04 | 2.45E−04 | 2.53E−06 |
| 3 | 1.47E−03 | 8.82E−07 | 4.54E−03 | 2.55E−04 | 3.69E−06 |
| 4 | 1.86E−02 | 5.08E−05 | 2.73E−02 | 3.92E−03 | 3.62E−05 |
| 5 | 9.61E−04 | 7.96E−07 | 9.64E−04 | 1.26E−02 | 1.69E−03 |
| 6 | 1.46E−02 | 1.31E−04 | 8.95E−03 | 2.06E−02 | 8.55E−03 |
| 7 | 4.85E−02 | 6.35E−04 | 2.98E−01 | 9.28E−02 | 9.01E−04 |
| 8 | 1.03E−01 | 9.82E−04 | 4.28E−01 | 1.13E−01 | 1.44E−03 |
| 9 | 1.66E−03 | 1.39E−01 | 5.16E−03 | 8.93E−02 | 1.08E−01 |
| 10 | 8.23E−01 | 8.17E−02 | 2.13E−01 | 9.61E−04 | 2.24E−02 |
| 11 | 4.37E−03 | 2.22E−01 | 9.03E−03 | 2.02E−01 | 2.91E−01 |
| 12 | 4.57E−02 | 7.49E−03 | 2.26E−01 | 4.29E−01 | 4.38E−02 |
| 13 | 5.50E−01 | 2.07E−01 | 1.23E−01 | 2.41E−03 | 4.88E−02 |
| 14 | 5.46E−01 | 9.58E−02 | 1.16E−01 | 2.46E−03 | 7.04E−02 |
| 15 | 9.73E−03 | 5.14E−01 | 9.59E−03 | 7.93E−01 | 8.06E−01 |
| 16 | 8.88E−01 | 8.87E−01 | 4.35E−02 | 7.96E−03 | 8.87E−01 |

TABLE 4f
Data for Enterobacter, particularly Enterobacter aerogenes (continued)

| | | | | |
|---|---|---|---|---|
| No. | Locus.Tag | Gene.Symbol | GenBank.Accession | Chromosome |

| | | | |
|---|---|---|---|
| 1 | EAE_18905 | | YP_004593967 |
| 2 | EAE_18910 | | YP_004593968 |
| 3 | EAE_20750 | | YP_004594334 |
| 4 | EAE_20680 | | YP_004594322 |
| 5 | EAE_10325 | | YP_004592260 |
| 6 | EAE_15945 | | YP_004593379 |
| 7 | EAE_24270 | | YP_004595026 |
| 8 | EAE_19175 | | YP_004594021 |
| 9 | ESA_03685 | | YP_001439725 |
| 10 | EAE_19000 | | YP_004593986 |
| 11 | EAE_21240 | | YP_004594432 |
| 12 | HMPREF9552_00257 | | ZP_07114469 |
| 13 | HMPREF9552_00255 | | ZP_07114467 |
| 14 | HMPREF9552_00247 | | ZP_07114459 |
| 15 | EAE_02625 | aphA | YP_004590739 |
| 16 | EAE_18950 | | YP_004593976 |

TABLE 4g
Data for Enterobacter, particularly
Enterobacter aerogenes (continued)

| No. | Start.Coord | End.Coord | Strand | Scaffold.ID |
|---|---|---|---|---|
| 1 | 4057377 | 4057994 | − | 650716122 |
| 2 | 4057994 | 4059313 | − | 650716122 |
| 3 | 4444576 | 4447206 | − | 650716122 |
| 4 | 4432502 | 4433842 | − | 650716122 |
| 5 | 2202535 | 2203893 | − | 650716122 |
| 6 | 3439556 | 3442066 | + | 650716122 |
| 7 | 5174177 | 5175436 | + | 650716122 |
| 8 | 4113374 | 4114306 | − | 650716122 |
| 9 | 3623778 | 3624146 | + | 640753067 |
| 10 | 4078519 | 4079427 | + | 650716122 |
| 11 | 4545796 | 4546983 | − | 650716122 |
| 12 | 14233 | 15036 | + | 648286894 |
| 13 | 12357 | 13376 | + | 648286894 |
| 14 | 7786 | 8685 | − | 648286894 |
| 15 | 557964 | 558680 | − | 650716122 |
| 16 | 4069127 | 4070251 | − | 650716122 |

TABLE 5a
Data for Enterobacter, particularly Enterobacter cloacae

| | Scaffold.External. | | |
|---|---|---|---|
| No. | Accession | Scaffold.Name | best_pv |
| 1 | NC_013509 | Edwardsiella tarda EIB202 plasmid pEIB202: NC_013509 | 5.49490970435242e−27 |
| 2 | NC_014107 | Enterobacter cloacae subsp. cloacae ATCC 13047 plasmid | 5.83313344212793e−25 |
| | | pECL_A: NC_014107 | |
| 3 | NC_009838 | Escherichia coli APEC O1 plasmid pAPEC-O1-R: NC_009838 | 5.83313344212793e−25 |
| 4 | NC_009838 | Escherichia coli APEC O1 plasmid pAPEC-O1-R: NC_009838 | 5.83313344212793e−25 |
| 5 | NC_014107 | Enterobacter cloacae subsp. cloacae ATCC 13047 plasmid | 1.25162242820201e−24 |
| | | pECL_A: NC_014107 | |
| 6 | NC_014107 | Enterobacter cloacae subsp. cloacae ATCC 13047 plasmid | 2.31689992231266e−24 |
| | | pECL_A: NC_014107 | |
| 7 | NC_014107 | Enterobacter cloacae subsp. cloacae ATCC 13047 plasmid | 2.46438038993034e−24 |
| | | pECL_A: NC_014107 | |
| 8 | NC_014107 | Enterobacter cloacae subsp. cloacae ATCC 13047 plasmid | 2.46438038993034e−24 |
| | | pECL_A: NC_014107 | |
| 9 | NZ_GG657370 | Citrobacter sp. 30_2 genomic scaffold supercont1.5: | 2.46438038993034e−24 |
| | | NZ_GG657370 | |
| 10 | NZ_GG657370 | Citrobacter sp. 30_2 genomic scaffold supercont1.5: | 1.59357817134779e−23 |
| | | NZ_GG657370 | |
| 11 | NC_009838 | Escherichia coli APEC O1 plasmid pAPEC-O1-R: NC_009838 | 1.59357817134779e−23 |
| 12 | NC_009778 | Enterobacter sakazakii ATCC BAA-894: NC_009778 | 2.83245228849161e−23 |
| 13 | NC_014107 | Enterobacter cloacae subsp. cloacae ATCC 13047 plasmid | 2.83245228849161e−23 |
| | | pECL_A: NC_014107 | |
| 14 | NZ_ADUD01000537 | Escherichia coli MS 196-1 E_coli196-1-1.0_Cont1482.1: | 9.1281268279348e−23 |
| | | NZ_ADUD01000537 | |
| 15 | NC_014121 | Enterobacter cloacae subsp. cloacae ATCC 13047 | 2.16502306009163e−22 |
| | | chromosome: NC_014121 | |
| 16 | NZ_ABFH01000001 | Salmonella enterica subsp. enterica serovar Virchow str. | 4.49434330590708e−22 |
| | | SL491, unfinished sequence: NZ_ABFH01000001 | |
| 17 | NC_004741 | Shigella flexneri 2a str. 2457T: NC_004741 | 1.02679325501045e−21 |
| 18 | NC_014121 | Enterobacter cloacae subsp. cloacae ATCC 13047 | 1.4942758061424e−21 |
| | | chromosome: NC_014121 | |
| 19 | NZ_AFBO01000168 | Klebsiella sp. MS 92-3 K_spMS92-3-1.0_Cont398.10: | 2.33876952186319e−21 |
| | | NZ_AFBO01000168 | |
| 20 | NC_014121 | Enterobacter cloacae subsp. cloacae ATCC 13047 | 7.16913512003583e−16 |
| | | chromosome: NC_014121 | |
| 21 | CP001383 | Shigella flexneri 2002017: CP001383 | 1.0532279235518e−15 |
| 22 | CP001637 | Escherichia coli DH1: CP001637 | 4.34714801916322e−15 |
| 23 | NZ_ADWT01000006 | Escherichia coli MS 124-1 E_coliMS124-1-1.0.1_Cont5.1: | 2.30933700130366e−14 |
| | | NZ_ADWT01000006 | |
| 24 | NZ_ADTR01000359 | Escherichia coli MS 21-1 E_coli21-1-1.0_Cont669.1: | 3.84987338572512e−13 |
| | | NZ_ADTR01000359 | |
| 25 | NC_014121 | Enterobacter cloacae subsp. cloacae ATCC 13047 | 5.47488441522503e−11 |
| | | chromosome: NC_014121 | |
| 26 | NZ_AAJV01000019 | Escherichia coli E22, unfinished sequence: | 9.06569667406974e−10 |
| | | NZ_AAJV01000019 | |
| 27 | FP929040 | Enterobacter cloacae subsp. cloacae NCTC 9394 draft | 1.83557061686134e−09 |
| | | genome.: FP929040 | |
| 28 | FP929040 | Enterobacter cloacae subsp. cloacae NCTC 9394 draft | 3.37768924887661e−09 |
| | | genome.: FP929040 | |
| 29 | FP929040 | Enterobacter cloacae subsp. cloacae NCTC 9394 draft | 5.84222468117604e−09 |
| | | genome.: FP929040 | |
| 30 | NZ_AFHR01000043 | Enterobacter hormaechei ATCC 49162 contig00043: | 8.80996353751187e−09 |
| | | NZ_AFHR01000043 | |
| 31 | FP929040 | Enterobacter cloacae subsp. cloacae NCTC 9394 draft | 1.29388450812614e−08 |
| | | genome.: FP929040 | |
| 32 | FP929040 | Enterobacter cloacae subsp. cloacae NCTC 9394 draft | 1.29388450812614e−08 |
| | | genome.: FP929040 | |
| 33 | FP929040 | Enterobacter cloacae subsp. cloacae NCTC 9394 draft | 1.29388450812614e−08 |
| | | genome.: FP929040 | |
| 34 | NZ_AFHR01000073 | Enterobacter hormaechei ATCC 49162 contig00073: | 1.29388450812614e−08 |
| | | NZ_AFHR01000073 | |
| 35 | NZ_AFHR01000073 | Enterobacter hormaechei ATCC 49162 contig00073: | 1.29388450812614e−08 |
| | | NZ_AFHR01000073 | |
| 36 | NZ_AFHR01000034 | Enterobacter hormaechei ATCC 49162 contig00034: | 1.29388450812614e−08 |
| | | NZ_AFHR01000034 | |
| 37 | NZ_AFHR01000073 | Enterobacter hormaechei ATCC 49162 contig00073: | 2.23051874499664e−08 |
| | | NZ_AFHR01000073 | |
| 38 | FP929040 | Enterobacter cloacae subsp. cloacae NCTC 9394 draft | 2.39066831640169e−08 |
| | | genome.: FP929040 | |
| 39 | NZ_AFHR01000062 | Enterobacter hormaechei ATCC 49162 contig00062: | 2.39066831640169e−08 |
| | | NZ_AFHR01000062 | |
| 40 | NC_014618 | Enterobacter cloacae SCF1 chromosome: NC_014618 | 2.76892470483105e−08 |
| 41 | NC_014618 | Enterobacter cloacae SCF1 chromosome: NC_014618 | 3.01843352491008e−08 |
| 42 | NZ_ABWM01000064 | Enterobacter cancerogenus ATCC 35316, unfinished | 4.40355882800235e−08 |
| | | sequence: NZ_ABWM01000064 | |
| 43 | NC_014121 | Enterobacter cloacae subsp. cloacae ATCC 13047 | 4.44394973624704e−08 |
| | | chromosome: NC_014121 | |
| 44 | NZ_GG657370 | Citrobacter sp. 30_2 genomic scaffold supercont1.5: | 5.11952538533182e−08 |
| | | NZ_GG657370 | |
| 45 | NC_014618 | Enterobacter cloacae SCF1 chromosome: NC_014618 | 5.14762193286233e−08 |
| 46 | NZ_AFHR01000015 | Enterobacter hormaechei ATCC 49162 contig00015: | 5.14762193286233e−08 |
| | | NZ_AFHR01000015 | |
| 47 | FP929040 | Enterobacter cloacae subsp. cloacae NCTC 9394 draft | 5.31046635969439e−08 |
| | | genome.: FP929040 | |
| 48 | FP929040 | Enterobacter cloacae subsp. cloacae NCTC 9394 draft | 5.31046635969439e−08 |
| | | genome.: FP929040 | |
| 49 | FP929040 | Enterobacter cloacae subsp. cloacae NCTC 9394 draft | 5.31046635969439e−08 |
| | | genome.: FP929040 | |
| 50 | FP929040 | Enterobacter cloacae subsp. cloacae NCTC 9394 draft | 5.31046635969439e−08 |
| | | genome.: FP929040 | |

TABLE 5b
Data for Enterobacter, particularly Enterobacter cloacae (continued)

| No. | sign_phenos | sign_phenos_class | best_pheno | best_pheno_class |
|---|---|---|---|---|
| 1 | AZT; CAX; CAZ; CFT; CP; CPE; CRM; ETP; GM; LVX; | aminoglycoside; fluoroquinolone; lactam; | T/S | other |
| | P/T; TO; T/S | other | | |
| 2 | AZT; CAX; CAZ; CFT; CP; CPE; CRM; GM; LVX; P/T; | aminoglycoside; fluoroquinolone; lactam; | T/S | other |
| | TO; T/S | other | | |
| 3 | AZT; CAX; CAZ; CFT; CP; CPE; CRM; GM; LVX; P/T; | aminoglycoside; fluoroquinolone; lactam; | T/S | other |
| | TO; T/S | other | | |
| 4 | AZT; CAX; CAZ; CFT; CP; CPE; CRM; GM; LVX; P/T; | aminoglycoside; fluoroquinolone; lactam; | T/S | other |
| | TO; T/S | other | | |
| 5 | AZT; CAX; CAZ; CFT; CP; CPE; CRM; GM; LVX; P/T; | aminoglycoside; fluoroquinolone; lactam; | T/S | other |
| | TO; T/S | other | | |
| 6 | AZT; CAX; CAZ; CFT; CP; CPE; ETP; GM; LVX; P/T; | aminoglycoside; fluoroquinolone; lactam; | T/S | other |
| | TO; T/S | other | | |
| 7 | AZT; CAX; CAZ; CFT; CP; CPE; GM; LVX; TO; T/S | aminoglycoside; fluoroquinolone; lactam; | T/S | other |
| | | other | | |
| 8 | AZT; CAX; CAZ; CFT; CP; CPE; GM; LVX; TO; T/S | aminoglycoside; fluoroquinolone; lactam; | T/S | other |
| | | other | | |
| 9 | AZT; CAX; CAZ; CFT; CP; CPE; CRM; GM; LVX; TO; | aminoglycoside; fluoroquinolone; lactam; | T/S | other |
| | T/S | other | | |
| 10 | AZT; CAX; CAZ; CFT; CP; CPE; CRM; GM; LVX; P/T; | aminoglycoside; fluoroquinolone; lactam; | T/S | other |
| | TO; T/S | other | | |
| 11 | AZT; CAX; CAZ; CFT; CP; CPE; GM; LVX; TO; T/S | aminoglycoside; fluoroquinolone; lactam; | T/S | other |
| | | other | | |
| 12 | AZT; CAX; CAZ; CFT; CP; CPE; CRM; GM; LVX; TO; | aminoglycoside; fluoroquinolone; lactam; | T/S | other |
| | T/S | other | | |
| 13 | AZT; CAX; CAZ; CFT; CP; CPE; GM; LVX; TO; T/S | aminoglycoside; fluoroquinolone; lactam; | T/S | other |
| | | other | | |
| 14 | AZT; CAX; CAZ; CFT; CP; CPE; CRM; ETP; GM; LVX; | aminoglycoside; fluoroquinolone; lactam; | GM | aminoglycoside |
| | P/T; TO; T/S | other | | |
| 15 | AZT; CAX; CAZ; CFT; CP; CPE; CRM; ETP; GM; LVX; | aminoglycoside; fluoroquinolone; lactam; | T/S | other |
| | P/T; TO; T/S | other | | |
| 16 | AZT; CAX; CAZ; CFT; CP; CPE; CRM; ETP; GM; LVX; | aminoglycoside; fluoroquinolone; lactam; | GM | aminoglycoside |
| | P/T; TO; T/S | other | | |
| 17 | AZT; CAX; CAZ; CFT; CP; CPE; CRM; GM; LVX; P/T; | aminoglycoside; fluoroquinolone; lactam; | GM | aminoglycoside |
| | TO; T/S | other | | |
| 18 | AZT; CAX; CAZ; CFT; CP; CPE; CRM; ETP; GM; LVX; | aminoglycoside; fluoroquinolone; lactam; | TO | aminoglycoside |
| | P/T; TO; T/S | other | | |
| 19 | AZT; CAX; CAZ; CFT; CP; CPE; CRM; ETP; GM; LVX; | aminoglycoside; fluoroquinolone; lactam; | T/S | other |
| | P/T; TO; T/S | other | | |
| 20 | AZT; CAX; CAZ; CFT; CP; CPE; CRM; GM; LVX; P/T; | aminoglycoside; fluoroquinolone; lactam; | TO | aminoglycoside |
| | TO; T/S | other | | |
| 21 | AZT; CAX; CAZ; CFT; CP; CPE; GM; LVX; P/T; TO; | aminoglycoside; fluoroquinolone; lactam; | T/S | other |
| | T/S | other | | |
| 22 | AZT; CAX; CAZ; CFT; CP; GM; LVX; P/T; TO; T/S | aminoglycoside; fluoroquinolone; lactam; | T/S | other |
| | | other | | |
| 23 | AZT; CAX; CAZ; CFT; CP; CPE; GM; LVX; P/T; TO; | aminoglycoside; fluoroquinolone; lactam; | GM | aminoglycoside |
| | T/S | other | | |
| 24 | AZT; CAX; CAZ; CFT; CP; CPE; GM; LVX; P/T; TO; | aminoglycoside; fluoroquinolone; lactam; | GM | aminoglycoside |
| | T/S | other | | |
| 25 | AZT; CAX; CAZ; CFT; CP; CPE; ETP; GM; LVX; P/T; | aminoglycoside; fluoroquinolone; lactam; | TO | aminoglycoside |
| | TO; T/S | other | | |
| 26 | AZT; CAX; CAZ; CFT; CP; CPE; ETP; GM; LVX; TO; | aminoglycoside; fluoroquinolone; lactam; | TO | aminoglycoside |
| | T/S | other | | |
| 27 | AZT; CAX; CAZ; CFT; CP; CPE; GM; LVX; P/T; TO; | aminoglycoside; fluoroquinolone; lactam; | CPE | lactam |
| | T/S | other | | |
| 28 | AZT; CAX; CAZ; CFT; CPE; GM; LVX; P/T; TO | aminoglycoside; fluoroquinolone; lactam | CPE | lactam |
| 29 | AZT; CAX; CAZ; CFT; CPE; GM; LVX; P/T; TO; T/S | aminoglycoside; fluoroquinolone; lactam; | CPE | lactam |
| | | other | | |
| 30 | AZT; CAZ; CFT; CP; CPE; GM; LVX; P/T; TO; T/S | aminoglycoside; fluoroquinolone; lactam; | CPE | lactam |
| | | other | | |
| 31 | AZT; CAX; CAZ; CPE; GM; LVX; P/T; TO; T/S | aminoglycoside; fluoroquinolone; lactam; | CPE | lactam |
| | | other | | |
| 32 | AZT; CAX; CAZ; CPE; GM; LVX; P/T; TO; T/S | aminoglycoside; fluoroquinolone; lactam; | CPE | lactam |
| | | other | | |
| 33 | AZT; CAX; CAZ; CPE; GM; LVX; P/T; TO; T/S | aminoglycoside; fluoroquinolone; lactam; | CPE | lactam |
| | | other | | |
| 34 | AZT; CAX; CAZ; CPE; GM; LVX; P/T; TO; T/S | aminoglycoside; fluoroquinolone; lactam; | CPE | lactam |
| | | other | | |
| 35 | AZT; CAX; CAZ; CPE; GM; LVX; P/T; TO; T/S | aminoglycoside; fluoroquinolone; lactam; | CPE | lactam |
| | | other | | |
| 36 | AZT; CAX; CAZ; CPE; GM; LVX; P/T; TO; T/S | aminoglycoside; fluoroquinolone; lactam; | CPE | lactam |
| | | other | | |
| 37 | AZT; CAX; CAZ; CPE; GM; LVX; P/T; TO; T/S | aminoglycoside; fluoroquinolone; lactam; | CPE | lactam |
| | | other | | |
| 38 | CP; CPE; GM; LVX; TO; T/S | aminoglycoside; fluoroquinolone; lactam; | CPE | lactam |
| | | other | | |
| 39 | CP; CPE; GM; LVX; TO; T/S | aminoglycoside; fluoroquinolone; lactam; | CPE | lactam |
| | | other | | |
| 40 | AZT; CAX; CAZ; CFT; CRM; GM; P/T; TO; T/S | aminoglycoside; lactam; other | T/S | other |
| 41 | AZT; CAX; CAZ; CFT; CP; CPE; ETP; GM; LVX; P/T; | aminoglycoside; fluoroquinolone; lactam; | CPE | lactam |
| | TO; T/S | other | | |
| 42 | CP; CPE; GM; LVX; TO; T/S | aminoglycoside; fluoroquinolone; lactam; | CPE | lactam |
| | | other | | |
| 43 | AZT; CAX; CAZ; CPE; GM; LVX; P/T; TO; T/S | aminoglycoside; fluoroquinolone; lactam; | CPE | lactam |
| | | other | | |
| 44 | AZT; CAX; CAZ; CFT; CP; GM; LVX; P/T; TO; T/S | aminoglycoside; fluoroquinolone; lactam; | T/S | other |
| | | other | | |
| 45 | CAX; CAZ; CPE; GM; LVX; P/T; TO; T/S | aminoglycoside; fluoroquinolone; lactam; | CPE | lactam |
| | | other | | |
| 46 | CP; CPE; GM; LVX; TO; T/S | aminoglycoside; fluoroquinolone; lactam; | CPE | lactam |
| | | other | | |
| 47 | AZT; CAX; CAZ; CPE; GM; LVX; P/T; TO; T/S | aminoglycoside; fluoroquinolone; lactam; | CPE | lactam |
| | | other | | |
| 48 | AZT; CAX; CAZ; CPE; GM; LVX; P/T; TO; T/S | aminoglycoside; fluoroquinolone; lactam; | CPE | lactam |
| | | other | | |
| 49 | AZT; CAX; CAZ; CPE; GM; LVX; P/T; TO; T/S | aminoglycoside; fluoroquinolone; lactam; | CPE | lactam |
| | | other | | |
| 50 | AZT; CAX; CAZ; CPE; GM; LVX; P/T; TO; T/S | aminoglycoside; fluoroquinolone; lactam; | CPE | lactam |
| | | other | | |

TABLE 5c
Data for Enterobacter, particularly Enterobacter cloacae (continued)

| No. | num_aminoglycoside | num_fluoroquinolone | num_lactam | num_other |
|---|---|---|---|---|
| 1 | 2 | 2 | 8 | 1 |
| 2 | 2 | 2 | 7 | 1 |
| 3 | 2 | 2 | 7 | 1 |
| 4 | 2 | 2 | 7 | 1 |
| 5 | 2 | 2 | 7 | 1 |
| 6 | 2 | 2 | 7 | 1 |
| 7 | 2 | 2 | 5 | 1 |
| 8 | 2 | 2 | 5 | 1 |
| 9 | 2 | 2 | 6 | 1 |
| 10 | 2 | 2 | 7 | 1 |
| 11 | 2 | 2 | 5 | 1 |
| 12 | 2 | 2 | 6 | 1 |
| 13 | 2 | 2 | 5 | 1 |
| 14 | 2 | 2 | 8 | 1 |
| 15 | 2 | 2 | 8 | 1 |
| 16 | 2 | 2 | 8 | 1 |
| 17 | 2 | 2 | 7 | 1 |
| 18 | 2 | 2 | 8 | 1 |
| 19 | 2 | 2 | 8 | 1 |
| 20 | 2 | 2 | 7 | 1 |
| 21 | 2 | 2 | 6 | 1 |
| 22 | 2 | 2 | 5 | 1 |
| 23 | 2 | 2 | 6 | 1 |
| 24 | 2 | 2 | 6 | 1 |
| 25 | 2 | 2 | 7 | 1 |
| 26 | 2 | 2 | 6 | 1 |
| 27 | 2 | 2 | 6 | 1 |
| 28 | 2 | 1 | 6 | 0 |
| 29 | 2 | 1 | 6 | 1 |
| 30 | 2 | 2 | 5 | 1 |
| 31 | 2 | 1 | 5 | 1 |
| 32 | 2 | 1 | 5 | 1 |
| 33 | 2 | 1 | 5 | 1 |
| 34 | 2 | 1 | 5 | 1 |
| 35 | 2 | 1 | 5 | 1 |
| 36 | 2 | 1 | 5 | 1 |
| 37 | 2 | 1 | 5 | 1 |
| 38 | 2 | 2 | 1 | 1 |
| 39 | 2 | 2 | 1 | 1 |
| 40 | 2 | 0 | 6 | 1 |
| 41 | 2 | 2 | 7 | 1 |
| 42 | 2 | 2 | 1 | 1 |
| 43 | 2 | 1 | 5 | 1 |
| 44 | 2 | 2 | 5 | 1 |
| 45 | 2 | 1 | 4 | 1 |
| 46 | 2 | 2 | 1 | 1 |
| 47 | 2 | 1 | 5 | 1 |
| 48 | 2 | 1 | 5 | 1 |
| 49 | 2 | 1 | 5 | 1 |
| 50 | 2 | 1 | 5 | 1 |

TABLE 5d
Data for Enterobacter, particularly Enterobacter cloacae (continued)

| No. | AZT_pv_adj | CAX_pv_adj | CAZ_pv_adj | CFT_pv_adj | CP_pv_adj |
|---|---|---|---|---|---|
| 1 | 3.58E−10 | 7.39E−09 | 2.80E−11 | 3.62E−09 | 5.27E−08 |
| 2 | 4.06E−06 | 7.65E−06 | 8.35E−07 | 8.00E−07 | 3.72E−07 |
| 3 | 4.06E−06 | 7.65E−06 | 8.35E−07 | 8.00E−07 | 3.72E−07 |
| 4 | 4.06E−06 | 7.65E−06 | 8.35E−07 | 8.00E−07 | 3.72E−07 |
| 5 | 8.70E−07 | 8.75E−06 | 5.21E−07 | 5.80E−07 | 1.18E−06 |
| 6 | 1.92E−12 | 2.08E−13 | 5.48E−12 | 1.92E−12 | 4.74E−09 |
| 7 | 2.20E−05 | 4.05E−05 | 4.94E−06 | 4.85E−06 | 7.75E−07 |
| 8 | 2.20E−05 | 4.05E−05 | 4.94E−06 | 4.85E−06 | 7.75E−07 |
| 9 | 6.62E−06 | 1.21E−05 | 1.34E−06 | 1.27E−06 | 3.72E−07 |
| 10 | 3.70E−06 | 6.73E−06 | 7.48E−07 | 7.08E−07 | 2.65E−06 |
| 11 | 3.46E−05 | 6.07E−05 | 7.99E−06 | 7.84E−06 | 7.16E−07 |
| 12 | 6.62E−06 | 1.21E−05 | 1.34E−06 | 1.27E−06 | 3.14E−06 |
| 13 | 2.20E−05 | 4.05E−05 | 4.94E−06 | 4.85E−06 | 7.75E−07 |
| 14 | 2.41E−09 | 3.66E−10 | 2.89E−10 | 1.33E−09 | 1.31E−11 |
| 15 | 6.79E−08 | 4.49E−08 | 1.13E−08 | 3.99E−08 | 4.19E−12 |
| 16 | 3.51E−09 | 1.67E−09 | 8.09E−10 | 8.35E−10 | 5.65E−12 |
| 17 | 3.11E−11 | 6.47E−11 | 7.01E−12 | 5.47E−11 | 1.53E−06 |
| 18 | 4.36E−12 | 6.39E−12 | 5.85E−13 | 5.48E−12 | 5.70E−07 |
| 19 | 6.79E−08 | 4.49E−08 | 1.13E−08 | 3.99E−08 | 3.61E−11 |
| 20 | 5.39E−09 | 1.23E−08 | 5.84E−09 | 6.70E−09 | 5.19E−07 |
| 21 | 3.36E−07 | 5.25E−07 | 5.19E−07 | 3.74E−06 | 3.91E−05 |
| 22 | 4.53E−07 | 8.07E−07 | 7.17E−07 | 6.01E−06 | 7.16E−05 |
| 23 | 1.62E−07 | 3.02E−07 | 2.83E−08 | 5.65E−08 | 9.46E−05 |
| 24 | 8.58E−09 | 1.75E−08 | 5.92E−08 | 2.01E−07 | 7.82E−07 |
| 25 | 5.28E−06 | 5.19E−07 | 1.32E−06 | 7.50E−07 | 1.27E−05 |
| 26 | 3.71E−05 | 2.06E−06 | 1.56E−05 | 9.73E−06 | 1.34E−04 |
| 27 | 2.41E−04 | 8.91E−05 | 8.91E−05 | 4.87E−04 | 5.72E−04 |
| 28 | 2.12E−03 | 7.75E−04 | 1.61E−03 | 5.63E−03 | 2.33E−02 |
| 29 | 3.60E−03 | 1.44E−03 | 1.46E−03 | 6.84E−03 | 1.58E−02 |
| 30 | 1.63E−03 | 1.30E−02 | 1.26E−03 | 9.88E−03 | 1.03E−03 |
| 31 | 8.73E−03 | 1.90E−03 | 3.60E−03 | 1.54E−02 | 2.18E−02 |
| 32 | 8.73E−03 | 1.90E−03 | 3.60E−03 | 1.54E−02 | 2.18E−02 |
| 33 | 8.73E−03 | 1.90E−03 | 3.60E−03 | 1.54E−02 | 2.18E−02 |
| 34 | 8.73E−03 | 1.90E−03 | 3.60E−03 | 1.54E−02 | 2.18E−02 |
| 35 | 8.73E−03 | 1.90E−03 | 3.60E−03 | 1.54E−02 | 2.18E−02 |
| 36 | 8.73E−03 | 1.90E−03 | 3.60E−03 | 1.54E−02 | 2.18E−02 |
| 37 | 9.06E−03 | 2.62E−03 | 4.92E−03 | 1.99E−02 | 2.22E−02 |
| 38 | 3.58E−02 | 1.04E−02 | 1.75E−02 | 5.31E−02 | 2.20E−05 |
| 39 | 5.33E−02 | 1.78E−02 | 2.85E−02 | 7.63E−02 | 6.85E−05 |
| 40 | 8.52E−05 | 3.90E−05 | 6.01E−06 | 2.49E−05 | 1.29E−02 |
| 41 | 4.11E−06 | 7.41E−04 | 4.26E−05 | 1.08E−03 | 6.16E−05 |
| 42 | 4.35E−02 | 1.36E−02 | 2.23E−02 | 6.48E−02 | 2.37E−05 |
| 43 | 8.79E−03 | 2.56E−03 | 4.80E−03 | 1.96E−02 | 2.95E−02 |
| 44 | 5.44E−03 | 1.03E−03 | 6.23E−04 | 1.01E−03 | 1.45E−04 |
| 45 | 1.49E−02 | 4.69E−03 | 8.56E−03 | 3.09E−02 | 3.86E−02 |
| 46 | 2.22E−01 | 6.69E−02 | 1.35E−01 | 3.01E−01 | 6.26E−04 |
| 47 | 8.87E−03 | 1.93E−03 | 3.69E−03 | 1.56E−02 | 3.05E−02 |
| 48 | 8.87E−03 | 1.93E−03 | 3.69E−03 | 1.56E−02 | 3.05E−02 |
| 49 | 8.87E−03 | 1.93E−03 | 3.69E−03 | 1.56E−02 | 3.05E−02 |
| 50 | 8.87E−03 | 1.93E−03 | 3.69E−03 | 1.56E−02 | 3.05E−02 |

TABLE 5e
Data for Enterobacter, particularly
Enterobacter cloacae (continued)

| No. | CPE_pv_adj | CRM_pv_adj | ETP_pv_adj | GM_pv_adj |
|---|---|---|---|---|
| 1 | 2.76E−09 | 1.20E−03 | 1.29E−04 | 3.48E−19 |
| 2 | 2.95E−05 | 2.74E−03 | 5.44E−02 | 5.95E−17 |
| 3 | 2.95E−05 | 2.74E−03 | 5.44E−02 | 5.95E−17 |
| 4 | 2.95E−05 | 2.74E−03 | 5.44E−02 | 5.95E−17 |
| 5 | 5.40E−05 | 2.87E−03 | 9.20E−02 | 6.93E−16 |
| 6 | 3.27E−08 | 3.20E−02 | 6.76E−03 | 1.07E−19 |
| 7 | 6.14E−05 | 1.06E−02 | 5.77E−02 | 2.51E−16 |
| 8 | 6.14E−05 | 1.06E−02 | 5.77E−02 | 2.51E−16 |
| 9 | 5.81E−05 | 2.77E−03 | 7.62E−02 | 4.00E−16 |
| 10 | 5.02E−05 | 3.99E−03 | 3.88E−02 | 2.55E−15 |
| 11 | 5.72E−05 | 1.10E−02 | 5.64E−02 | 1.59E−16 |
| 12 | 5.81E−05 | 6.94E−03 | 4.04E−02 | 4.15E−15 |
| 13 | 1.93E−05 | 1.06E−02 | 5.77E−02 | 2.51E−16 |
| 14 | 5.33E−09 | 5.60E−04 | 8.61E−04 | 9.13E−23 |
| 15 | 1.62E−07 | 1.89E−03 | 7.13E−04 | 4.13E−22 |
| 16 | 1.55E−08 | 1.01E−03 | 1.04E−03 | 4.49E−22 |
| 17 | 5.31E−08 | 9.22E−03 | 1.04E−02 | 1.03E−21 |
| 18 | 1.37E−08 | 1.77E−03 | 7.24E−03 | 1.52E−21 |
| 19 | 3.31E−08 | 1.89E−03 | 6.74E−03 | 5.26E−21 |
| 20 | 5.79E−05 | 2.43E−03 | 5.85E−02 | 1.07E−14 |
| 21 | 8.87E−03 | 1.21E−02 | 2.44E−01 | 2.97E−08 |
| 22 | 1.76E−02 | 1.63E−02 | 2.41E−01 | 7.76E−08 |
| 23 | 2.82E−06 | 7.01E−02 | 5.22E−02 | 2.31E−14 |
| 24 | 3.31E−04 | 2.06E−02 | 1.04E−02 | 3.85E−13 |
| 25 | 1.65E−04 | 1.14E−01 | 1.77E−03 | 5.27E−08 |
| 26 | 1.92E−04 | 5.27E−02 | 1.45E−03 | 1.24E−08 |
| 27 | 1.84E−09 | 3.12E−02 | 9.47E−02 | 3.86E−04 |
| 28 | 3.38E−09 | 2.11E−01 | 1.66E−01 | 6.74E−04 |
| 29 | 5.84E−09 | 6.96E−02 | 2.65E−01 | 9.71E−04 |
| 30 | 8.81E−09 | 1.75E−01 | 1.27E−01 | 2.57E−04 |
| 31 | 1.29E−08 | 8.58E−02 | 3.28E−01 | 1.48E−03 |
| 32 | 1.29E−08 | 8.58E−02 | 3.28E−01 | 1.48E−03 |
| 33 | 1.29E−08 | 8.58E−02 | 3.28E−01 | 1.48E−03 |
| 34 | 1.29E−08 | 8.58E−02 | 3.28E−01 | 1.48E−03 |
| 35 | 1.29E−08 | 8.58E−02 | 3.28E−01 | 1.48E−03 |
| 36 | 1.29E−08 | 8.58E−02 | 3.28E−01 | 1.48E−03 |
| 37 | 2.23E−08 | 8.52E−02 | 3.28E−01 | 1.51E−03 |
| 38 | 2.39E−08 | 4.38E−01 | 5.64E−02 | 4.16E−03 |
| 39 | 2.39E−08 | 4.38E−01 | 5.64E−02 | 4.16E−03 |
| 40 | 1.69E−01 | 6.82E−04 | 7.60E−01 | 2.98E−04 |
| 41 | 3.02E−08 | 3.74E−02 | 3.81E−04 | 1.38E−04 |
| 42 | 4.40E−08 | 3.77E−01 | 5.72E−02 | 4.38E−03 |
| 43 | 4.44E−08 | 6.75E−02 | 3.31E−01 | 2.17E−03 |
| 44 | 2.31E−02 | 4.84E−02 | 6.10E−02 | 1.15E−06 |
| 45 | 5.15E−08 | 8.55E−02 | 2.60E−01 | 3.19E−03 |
| 46 | 5.15E−08 | 7.80E−02 | 4.73E−01 | 1.13E−03 |
| 47 | 5.31E−08 | 1.08E−01 | 2.67E−01 | 2.37E−03 |
| 48 | 5.31E−08 | 1.08E−01 | 2.67E−01 | 2.37E−03 |
| 49 | 5.31E−08 | 1.08E−01 | 2.67E−01 | 2.37E−03 |
| 50 | 5.31E−08 | 1.08E−01 | 2.67E−01 | 2.37E−03 |

TABLE 5f
Data for Enterobacter, particularly
Enterobacter cloacae (continued)

| No. | LVX_pv_adj | P/T_pv_adj | TO_pv_adj | T/S_pv_adj |
|---|---|---|---|---|
| 1 | 8.81E−09 | 1.52E−04 | 2.06E−19 | 5.49E−27 |
| 2 | 2.74E−08 | 6.34E−03 | 2.69E−20 | 5.83E−25 |
| 3 | 2.74E−08 | 6.34E−03 | 2.69E−20 | 5.83E−25 |
| 4 | 2.74E−08 | 6.34E−03 | 2.69E−20 | 5.83E−25 |
| 5 | 5.07E−07 | 6.25E−03 | 9.93E−19 | 1.25E−24 |
| 6 | 1.21E−11 | 4.05E−05 | 5.70E−21 | 2.32E−24 |
| 7 | 5.15E−08 | 1.58E−02 | 1.59E−19 | 2.46E−24 |
| 8 | 5.15E−08 | 1.58E−02 | 1.59E−19 | 2.46E−24 |
| 9 | 7.06E−08 | 1.10E−02 | 1.73E−19 | 2.46E−24 |
| 10 | 3.37E−07 | 8.08E−03 | 1.06E−18 | 1.59E−23 |
| 11 | 4.09E−08 | 2.07E−02 | 9.23E−20 | 1.59E−23 |
| 12 | 3.72E−07 | 1.10E−02 | 1.95E−18 | 2.83E−23 |
| 13 | 5.15E−08 | 1.58E−02 | 1.59E−19 | 2.83E−23 |
| 14 | 1.31E−11 | 2.13E−07 | 5.43E−22 | 1.85E−22 |
| 15 | 4.19E−12 | 8.40E−07 | 8.04E−22 | 2.17E−22 |
| 16 | 5.65E−12 | 5.70E−07 | 2.63E−21 | 1.03E−21 |
| 17 | 1.00E−08 | 4.60E−04 | 1.44E−21 | 4.11E−18 |
| 18 | 2.49E−09 | 1.68E−04 | 1.49E−21 | 4.99E−18 |
| 19 | 4.19E−12 | 8.40E−07 | 7.92E−21 | 2.34E−21 |
| 20 | 6.44E−09 | 2.28E−04 | 7.17E−16 | 1.02E−15 |
| 21 | 8.28E−05 | 6.44E−04 | 3.80E−10 | 1.05E−15 |
| 22 | 1.82E−04 | 9.24E−04 | 1.96E−09 | 4.35E−15 |
| 23 | 1.55E−05 | 1.40E−04 | 3.69E−14 | 6.50E−11 |
| 24 | 7.82E−07 | 1.61E−04 | 8.30E−12 | 1.21E−11 |
| 25 | 3.34E−06 | 3.34E−03 | 5.47E−11 | 4.91E−08 |
| 26 | 6.31E−06 | 1.70E−02 | 9.07E−10 | 5.12E−08 |
| 27 | 2.79E−05 | 2.12E−03 | 1.68E−04 | 4.44E−04 |
| 28 | 2.35E−03 | 8.21E−03 | 2.41E−03 | 3.78E−02 |
| 29 | 1.41E−03 | 2.25E−03 | 9.05E−04 | 1.74E−03 |
| 30 | 1.97E−05 | 1.77E−03 | 8.04E−05 | 5.50E−04 |
| 31 | 2.08E−03 | 5.50E−03 | 1.36E−03 | 3.40E−03 |
| 32 | 2.08E−03 | 5.50E−03 | 1.36E−03 | 3.40E−03 |
| 33 | 2.08E−03 | 5.50E−03 | 1.36E−03 | 3.40E−03 |
| 34 | 2.08E−03 | 5.50E−03 | 1.36E−03 | 3.40E−03 |
| 35 | 2.08E−03 | 5.50E−03 | 1.36E−03 | 3.40E−03 |
| 36 | 2.08E−03 | 5.50E−03 | 1.36E−03 | 3.40E−03 |
| 37 | 2.10E−03 | 7.39E−03 | 1.42E−03 | 3.42E−03 |
| 38 | 1.40E−06 | 4.00E−02 | 2.39E−03 | 8.45E−04 |
| 39 | 5.74E−06 | 7.03E−02 | 2.39E−03 | 8.45E−04 |
| 40 | 1.29E−02 | 8.60E−03 | 1.87E−05 | 2.77E−08 |
| 41 | 6.73E−06 | 8.68E−06 | 4.49E−05 | 2.53E−04 |
| 42 | 1.52E−06 | 4.87E−02 | 2.44E−03 | 8.72E−04 |
| 43 | 3.07E−03 | 5.47E−03 | 1.99E−03 | 2.25E−03 |
| 44 | 1.45E−04 | 6.88E−03 | 4.69E−06 | 5.12E−08 |
| 45 | 3.22E−03 | 9.52E−03 | 2.86E−03 | 3.22E−03 |
| 46 | 6.11E−05 | 1.82E−01 | 2.53E−04 | 3.05E−04 |
| 47 | 3.31E−03 | 5.73E−03 | 2.11E−03 | 5.16E−03 |
| 48 | 3.31E−03 | 5.73E−03 | 2.11E−03 | 5.16E−03 |
| 49 | 3.31E−03 | 5.73E−03 | 2.11E−03 | 5.16E−03 |
| 50 | 3.31E−03 | 5.73E−03 | 2.11E−03 | 5.16E−03 |

TABLE 5g
Data for Enterobacter, particularly Enterobacter cloacae (continued)

| No. | Locus.Tag | Gene.Symbol | GenBank.Accession | Chromosome |
|---|---|---|---|---|
| 1 | ETAE_p041 | folP | YP_003297635 | plasmid pEIB202 |
| 2 | ECL_A197 | | YP_003602692 | plasmid pECL_A |
| 3 | APECO1_O1R60 | terY1 | YP_001481413 | plasmid pAPEC-O1-R |
| 4 | APECO1_O1R58 | terY2 | YP_001481411 | plasmid pAPEC-O1-R |
| 5 | ECL_A215 | | YP_003602710 | plasmid pECL_A |
| 6 | ECL_A245 | | YP_003602738 | plasmid pECL_A |
| 7 | ECL_A211 | | YP_003602706 | plasmid pECL_A |
| 8 | ECL_A213 | | YP_003602708 | plasmid pECL_A |
| 9 | CSAG_04328 | | ZP_04559059 | |
| 10 | CSAG_04340 | | ZP_04559071 | |
| 11 | APECO1_O1R72 | terD | YP_001481425 | plasmid pAPEC-O1-R |
| 12 | ESA_01791 | | YP_001437881 | |
| 13 | ECL_A210 | | YP_003602705 | plasmid pECL_A |
| 14 | HMPREF9551_04611 | | ZP_07191964 | |
| 15 | ECL_03822 | merP | YP_003614305 | |
| 16 | Salmoentericaenterica_010100000105 | | ZP_02702049 | |
| 17 | S2707 | parA | NP_838057 | |
| 18 | ECL_03815 | | YP_003614298 | |
| 19 | HMPREF9538_01300 | | ZP_08303641 | |
| 20 | ECL_00479 | | YP_003610994 | |
| 21 | SFxv_1737 | | ADA73942 | |
| 22 | EcDH1_2078 | | ACX39734 | |
| 23 | HMPREF9347_01086 | | ZP_07208640 | |
| 24 | HMPREF9530_03894 | | ZP_07153755 | |
| 25 | ECL_00391 | | YP_003610906 | |
| 26 | EcolE2_01002790 | | ZP_00728858 | |
| 27 | ENC_13410 | | CBK85143 | |
| 28 | ENC_03420 | | CBK84384 | |
| 29 | ENC_31170 | | CBK86512 | |
| 30 | HMPREF9086_2435 | | ZP_08498173 | |
| 31 | ENC_11900 | | CBK85029 | |
| 32 | ENC_02650 | | CBK84321 | |
| 33 | ENC_02700 | | CBK84325 | |
| 34 | HMPREF9086_4455 | amsH2 | ZP_08500191 | |
| 35 | HMPREF9086_4454 | yccY2 | ZP_08500190 | |
| 36 | HMPREF9086_2100 | yjdF | ZP_08497838 | |
| 37 | HMPREF9086_4460 | ligA2 | ZP_08500196 | |
| 38 | ENC_28850 | | CBK86326 | |
| 39 | HMPREF9086_3524 | gatAX | ZP_08499260 | |
| 40 | Entcl_2219 | | YP_003941757 | |
| 41 | Entcl_2207 | | YP_003941745 | |
| 42 | ENTCAN_03923 | | ZP_03284103 | |
| 43 | ECL_01132 | | YP_003611642 | |
| 44 | CSAG_04209 | | ZP_04558940 | |
| 45 | Entcl_0777 | | YP_003940336 | |
| 46 | HMPREF9086_0855 | | ZP_08496597 | |
| 47 | ENC_21030 | | CBK85719 | |
| 48 | ENC_20990 | | CBK85716 | |
| 49 | ENC_21000 | | CBK85717 | |
| 50 | ENC_21040 | | CBK85720 | |

TABLE 5h
Data for Enterobacter, particularly
Enterobacter cloacae (continued)

| No. | Start.Coord | End.Coord | Strand | Scaffold.ID |
|---|---|---|---|---|
| 1 | 32101 | 32916 | − | 646311959 |
| 2 | 142295 | 142936 | − | 646564617 |
| 3 | 68792 | 69511 | − | 640753093 |
| 4 | 67490 | 68131 | − | 640753093 |
| 5 | 156077 | 157318 | + | 646564617 |
| 6 | 176747 | 177670 | − | 646564617 |
| 7 | 153338 | 154378 | + | 646564617 |
| 8 | 155073 | 155648 | + | 646564617 |
| 9 | 377133 | 378173 | − | 646206736 |
| 10 | 388284 | 389441 | + | 646206736 |
| 11 | 80263 | 80841 | + | 640753093 |
| 12 | 1731242 | 1732198 | − | 640753067 |
| 13 | 152860 | 153315 | + | 646564617 |
| 14 | 97 | 531 | − | 648293053 |
| 15 | 3908948 | 3909223 | + | 646564624 |
| 16 | 16271 | 16636 | − | 641778223 |
| 17 | 2602155 | 2602892 | − | 637000219 |
| 18 | 3903410 | 3903712 | + | 646564624 |
| 19 | 6404 | 6640 | + | 651333085 |
| 20 | 482851 | 484329 | − | 646564624 |
| 21 | 1636971 | 1637258 | − | 646862363 |
| 22 | 2232606 | 2232845 | + | 646862383 |
| 23 | 2726 | 5692 | − | 648294279 |
| 24 | 1918 | 3231 | − | 648290583 |
| 25 | 409807 | 410283 | + | 646564624 |
| 26 | 14395 | 15375 | + | 638358747 |
| 27 | 1397946 | 1398188 | − | 650378029 |
| 28 | 360699 | 361856 | + | 650378029 |
| 29 | 3151035 | 3151685 | − | 650378029 |
| 30 | 192614 | 193519 | + | 651335147 |
| 31 | 1250898 | 1252163 | + | 650378029 |
| 32 | 274155 | 276332 | − | 650378029 |
| 33 | 280790 | 281458 | − | 650378029 |
| 34 | 111547 | 112680 | − | 651335177 |
| 35 | 111116 | 111559 | − | 651335177 |
| 36 | 33364 | 34074 | + | 651335138 |
| 37 | 117314 | 119053 | − | 651335177 |
| 38 | 2904606 | 2905577 | − | 650378029 |
| 39 | 8440 | 9756 | − | 651335166 |
| 40 | 2349542 | 2349967 | + | 649633020 |
| 41 | 2334593 | 2335594 | − | 649633020 |
| 42 | 28272 | 29567 | − | 643006662 |
| 43 | 1168855 | 1169388 | + | 646564624 |
| 44 | 254005 | 255187 | − | 646206736 |
| 45 | 812618 | 813094 | + | 649633020 |
| 46 | 13947 | 15560 | − | 651335119 |
| 47 | 2148896 | 2151391 | + | 650378029 |
| 48 | 2145171 | 2145458 | + | 650378029 |
| 49 | 2145486 | 2146736 | + | 650378029 |
| 50 | 2151395 | 2152267 | − | 650378029 |

TABLE 6a
Data for Enterobacter, particularly Enterobacter cloacae

| | Scaffold.external. | | |
|---|---|---|---|
| No. | Accession | Scaffold.Name | best_pv |
| 1 | NC_013509 | Edwardsiella tarda EIB202 plasmid pEIB202: NC_013509 | 5.49490970435242e−27 |
| 2 | NC_014107 | Enterobacter cloacae subsp. cloacae ATCC 13047 plasmid | 5.83313344212793e−25 |
| | | pECL_A: NC_014107 | |
| 3 | NC_009838 | Escherichia coli APEC O1 plasmid pAPEC-O1-R: NC_009838 | 5.83313344212793e−25 |
| 4 | NC_009838 | Escherichia coli APEC O1 plasmid pAPEC-O1-R: NC_009838 | 5.83313344212793e−25 |
| 5 | NC_014107 | Enterobacter cloacae subsp. cloacae ATCC 13047 plasmid | 1.25162242820201e−24 |
| | | pECL_A: NC_014107 | |
| 6 | NC_014107 | Enterobacter cloacae subsp. cloacae ATCC 13047 plasmid | 2.31689992231266e−24 |
| | | pECL_A: NC_014107 | |
| 7 | NC_014107 | Enterobacter cloacae subsp. cloacae ATCC 13047 plasmid | 2.46438038993034e−24 |
| | | pECL_A: NC_014107 | |
| 8 | NC_014107 | Enterobacter cloacae subsp. cloacae ATCC 13047 plasmid | 2.46438038993034e−24 |
| | | pECL_A: NC_014107 | |
| 9 | NZ_GG657370 | Citrobacter sp. 30_2 genomic scaffold supercont1.5: | 2.46438038993034e−24 |
| | | NZ_GG657370 | |
| 10 | NZ_GG657370 | Citrobacter sp. 30_2 genomic scaffold supercont1.5: | 1.59357817134779e−23 |
| | | NZ_GG657370 | |
| 11 | NC_009838 | Escherichia coli APEC O1 plasmid pAPEC-O1-R: NC_009838 | 1.59357817134779e−23 |
| 12 | NC_009778 | Enterobacter sakazakii ATCC BAA-894: NC_009778 | 2.83245228849161e−23 |
| 13 | NC_014107 | Enterobacter cloacae subsp. cloacae ATCC 13047 plasmid | 2.83245228849161e−23 |
| | | pECL_A: NC_014107 | |
| 14 | NZ_ADUD01000537 | Escherichia coli MS 196-1 E_coli196-1-1.0_Cont1482.1: | 9.1281268279348e−23 |
| | | NZ_ADUD01000537 | |
| 15 | NC_014121 | Enterobacter cloacae subsp. cloacae ATCC 13047 | 2.16502306009163e−22 |
| | | chromosome: NC_014121 | |
| 16 | NZ_ABFH01000001 | Salmonella enterica subsp. enterica serovar Virchow | 4.49434330590708e−22 |
| | | str. SL491, unfinished sequence: NZ_ABFH01000001 | |
| 17 | NC_004741 | Shigella flexneri 2a str. 2457T: NC_004741 | 1.02679325501045e−21 |
| 18 | NC_014121 | Enterobacter cloacae subsp. cloacae ATCC 13047 | 1.4942758061424e−21 |
| | | chromosome: NC_014121 | |
| 19 | NZ_AFBO01000168 | Klebsiella sp. MS 92-3 K_spMS92-3-1.0_Cont398.10: | 2.33876952186319e−21 |
| | | NZ_AFBO01000168 | |
| 20 | NC_014121 | Enterobacter cloacae subsp. cloacae ATCC 13047 | 7.16913512003583e−16 |
| | | chromosome: NC_014121 | |
| 21 | CP001383 | Shigella flexneri 2002017: CP001383 | 1.0532279235518e−15 |
| 22 | CP001637 | Escherichia coli DH1: CP001637 | 4.34714801916322e−15 |
| 23 | NZ_ADWT01000006 | Escherichia coli MS 124-1 E_coliMS124-1.1.0.1_Cont5.1: | 2.30933700130366e−14 |
| | | NZ_ADWT01000006 | |
| 24 | NZ_ADTR01000359 | Escherichia coli MS 21-1 E_coli21-1-1.0_Cont669.1: | 3.84987338572512e−13 |
| | | NZ_ADTR01000359 | |
| 25 | NC_014121 | Enterobacter cloacae subsp. cloacae ATCC 13047 | 5.47488441522503e−11 |
| | | chromosome: NC_014121 | |
| 26 | NZ_AAJV01000019 | Escherichia coli E22, unfinished sequence: | 9.06569667406974e−10 |
| | | NZ_AAJV01000019 | |
| 27 | FP929040 | Enterobacter cloacae subsp. cloacae NCTC 9394 draft | 1.83557061686134e−09 |
| | | genome.: FP929040 | |
| 28 | FP929040 | Enterobacter cloacae subsp. cloacae NCTC 9394 draft | 3.37768924887661e−09 |
| | | genome.: FP929040 | |
| 29 | FP929040 | Enterobacter cloacae subsp. cloacae NCTC 9394 draft | 5.84222468117604e−09 |
| | | genome.: FP929040 | |
| 30 | NZ_AFHR01000043 | Enterobacter hormaechei ATCC 49162 contig00043: | 8.80996353751187e−09 |
| | | NZ_AFHR01000043 | |
| 31 | FP929040 | Enterobacter cloacae subsp. cloacae NCTC 9394 draft | 1.29388450812614e−08 |
| | | genome.: FP929040 | |
| 32 | FP929040 | Enterobacter cloacae subsp. cloacae NCTC 9394 draft | 1.29388450812614e−08 |
| | | genome.: FP929040 | |
| 33 | FP929040 | Enterobacter cloacae subsp. cloacae NCTC 9394 draft | 1.29388450812614e−08 |
| | | genome.: FP929040 | |
| 34 | NZ_AFHR01000073 | Enterobacter hormaechei ATCC 49162 contig00073: | 1.29388450812614e−08 |
| | | NZ_AFHR01000073 | |
| 35 | NZ_AFHR01000073 | Enterobacter hormaechei ATCC 49162 contig00073: | 1.29388450812614e−08 |
| | | NZ_AFHR01000073 | |
| 36 | NZ_AFHR01000034 | Enterobacter hormaechei ATCC 49162 contig00034: | 1.29388450812614e−08 |
| | | NZ_AFHR01000034 | |
| 37 | NZ_AFHR01000073 | Enterobacter hormaechei ATCC 49162 contig00073: | 2.23051874499664e−08 |
| | | NZ_AFHR01000073 | |
| 38 | FP929040 | Enterobacter cloacae subsp. cloacae NCTC 9394 draft | 2.39066831640169e−08 |
| | | genome.: FP929040 | |
| 39 | NZ_AFHR01000062 | Enterobacter hormaechei ATCC 49162 contig00062: | 2.39066831640169e−08 |
| | | NZ_AFHR01000062 | |
| 40 | NC_014618 | Enterobacter cloacae SCF1 chromosome: NC_014618 | 2.76892470483105e−08 |
| 41 | NC_014618 | Enterobacter cloacae SCF1 chromosome: NC_014618 | 3.01843352491008e−08 |
| 42 | NZ_ABWM01000064 | Enterobacter cancerogenus ATCC 35316, unfinished | 4.40355882800235e−08 |
| | | sequence: NZ_ABWM01000064 | |
| 43 | NC_014121 | Enterobacter cloacae subsp. cloacae ATCC 13047 | 4.44394973624704e−08 |
| | | chromosome: NC_014121 | |
| 44 | NZ_GG657370 | Citrobacter sp. 30_2 genomic scaffold supercont1.5: | 5.11952538533182e−08 |
| | | NZ_GG657370 | |
| 45 | NC_014618 | Enterobacter cloacae SCF1 chromosome: NC_014618 | 5.14762193286233e−08 |
| 46 | NZ_AFHR01000015 | Enterobacter hormaechei ATCC 49162 contig00015: | 5.14762193286233e−08 |
| | | NZ_AFHR01000015 | |
| 47 | FP929040 | Enterobacter cloacae subsp. cloacae NCTC 9394 draft | 5.31046635969439e−08 |
| | | genome.: FP929040 | |
| 48 | FP929040 | Enterobacter cloacae subsp. cloacae NCTC 9394 draft | 5.31046635969439e−08 |
| | | genome.: FP929040 | |
| 49 | FP929040 | Enterobacter cloacae subsp. cloacae NCTC 9394 draft | 5.31046635969439e−08 |
| | | genome.: FP929040 | |
| 50 | FP929040 | Enterobacter cloacae subsp. cloacae NCTC 9394 draft | 5.31046635969439e−08 |
| | | genome.: FP929040 | |

TABLE 6b
Data for Enterobacter, particularly Enterobacter cloacae (continued)

| No. | sign_phenos | sign_phenos_class | best_pheno | best_pheno_class |
|---|---|---|---|---|
| 1 | AZT; CAX; CAZ; CFT; CP; CPE; CRM; ETP; GM; LVX; P/T; | aminoglycoside; fluoroquinolone; lactam; | T/S | other |
| | TO; T/S | other | | |
| 2 | AZT; CAX; CAZ; CFT; CP; CPE; CRM; GM; LVX; P/T; | aminoglycoside; fluoroquinolone; lactam; | T/S | other |
| | TO; T/S | other | | |
| 3 | AZT; CAX; CAZ; CFT; CP; CPE; CRM; GM; LVX; P/T; | aminoglycoside; fluoroquinolone; lactam; | T/S | other |
| | TO; T/S | other | | |
| 4 | AZT; CAX; CAZ; CFT; CP; CPE; CRM; GM; LVX; P/T; | aminoglycoside; fluoroquinolone; lactam; | T/S | other |
| | TO; T/S | other | | |
| 5 | AZT; CAX; CAZ; CFT; CP; CPE; CRM; GM; LVX; P/T; | aminoglycoside; fluoroquinolone; lactam; | T/S | other |
| | TO; T/S | other | | |
| 6 | AZT; CAX; CAZ; CFT; CP; CPE; ETP; GM; LVX; P/T; | aminoglycoside; fluoroquinolone; lactam; | T/S | other |
| | TO; T/S | other | | |
| 7 | AZT; CAX; CAZ; CFT; CP; CPE; GM; LVX; TO; T/S | aminoglycoside; fluoroquinolone; lactam; | T/S | other |
| | | other | | |
| 8 | AZT; CAX; CAZ; CFT; CP; CPE; GM; LVX; TO; T/S | aminoglycoside; fluoroquinolone; lactam; | T/S | other |
| | | other | | |
| 9 | AZT; CAX; CAZ; CFT; CP; CPE; CRM; GM; LVX; TO; T/S | aminoglycoside; fluoroquinolone; lactam; | T/S | other |
| | | other | | |
| 10 | AZT; CAX; CAZ; CFT; CP; CPE; CRM; GM; LVX; P/T; | aminoglycoside; fluoroquinolone; lactam; | T/S | other |
| | TO; T/S | other | | |
| 11 | AZT; CAX; CAZ; CFT; CP; CPE; GM; LVX; TO; T/S | aminoglycoside; fluoroquinolone; lactam; | T/S | other |
| | | other | | |
| 12 | AZT; CAX; CAZ; CFT; CP; CPE; CRM; GM; LVX; TO; T/S | aminoglycoside; fluoroquinolone; lactam; | T/S | other |
| | | other | | |
| 13 | AZT; CAX; CAZ; CFT; CP; CPE; GM; LVX; TO; T/S | aminoglycoside; fluoroquinolone; lactam; | T/S | other |
| | | other | | |
| 14 | AZT; CAX; CAZ; CFT; CP; CPE; CRM; ETP; GM; LVX; P/T; | aminoglycoside; fluoroquinolone; lactam; | GM | aminoglycoside |
| | TO; T/S | other | | |
| 15 | AZT; CAX; CAZ; CFT; CP; CPE; CRM; ETP; GM; LVX; P/T; | aminoglycoside; fluoroquinolone; lactam; | T/S | other |
| | TO; T/S | other | | |
| 16 | AZT; CAX; CAZ; CFT; CP; CPE; CRM; ETP; GM; LVX; P/T; | aminoglycoside; fluoroquinolone; lactam; | GM | aminoglycoside |
| | TO; T/S | other | | |
| 17 | AZT; CAX; CAZ; CFT; CP; CPE; CRM; GM; LVX; P/T; | aminoglycoside; fluoroquinolone; lactam; | GM | aminogiycoside |
| | TO; T/S | other | | |
| 18 | AZT; CAX; CAZ; CFT; CP; CPE; CRM; ETP; GM; LVX; P/T; | aminoglycoside; fluoroquinolone; lactam; | TO | aminogiycoside |
| | TO; T/S | other | | |
| 19 | AZT; CAX; CAZ; CFT; CP; CPE; CRM; ETP; GM; LVX; P/T; | aminoglycoside; fluoroquinolone; lactam; | T/S | other |
| | TO; T/S | other | | |
| 20 | AZT; CAX; CAZ; CFT; CP; CPE; CRM; GM; LVX; P/T; | aminoglycoside; fluoroquinolone; lactam; | TO | aminogiycoside |
| | TO; T/S | other | | |
| 21 | AZT; CAX; CAZ; CFT; CP; CPE; GM; LVX; P/T; TO; T/S | aminoglycoside; fluoroquinolone; lactam; | T/S | other |
| | | other | | |
| 22 | AZT; CAX; CAZ; CFT; CP; GM; LVX; P/T; TO; T/S | aminoglycoside; fluoroquinolone; lactam; | T/S | other |
| | | other | | |
| 23 | AZT; CAX; CAZ; CFT; CP; CPE; GM; LVX; P/T; TO; T/S | aminoglycoside; fluoroquinolone; lactam; | GM | aminogiycoside |
| | | other | | |
| 24 | AZT; CAX; CAZ; CFT; CP; CPE; GM; LVX; P/T; TO; T/S | aminoglycoside; fluoroquinolone; lactam; | GM | aminogiycoside |
| | | other | | |
| 25 | AZT; CAX; CAZ; CFT; CP; CPE; ETP; GM; LVX; P/T; | aminoglycoside; fluoroquinolone; lactam; | TO | aminogiycoside |
| | TO; T/S | other | | |
| 26 | AZT; CAX; CAZ; CFT; CP; CPE; ETP; GM; LVX; TO; T/S | aminoglycoside; fluoroquinolone; lactam; | TO | aminogiycoside |
| | | other | | |
| 27 | AZT; CAX; CAZ; CFT; CP; CPE; GM; LVX; P/T; TO; T/S | aminoglycoside; fluoroquinolone; lactam; | CPE | lactam |
| | | other | | |
| 28 | AZT; CAX; CAZ; CFT; CPE; GM; LVX; P/T; TO | aminoglycoside; fluoroquinolone; lactam | CPE | lactam |
| 29 | AZT; CAX; CAZ; CFT; CPE; GM; LVX; P/T; TO; T/S | aminoglycoside; fluoroquinolone; lactam; | CPE | lactam |
| | | other | | |
| 30 | AZT; CAZ; CFT; CP; CPE; GM; LVX; P/T; TO; T/S | aminoglycoside; fluoroquinolone; lactam; | CPE | lactam |
| | | other | | |
| 31 | AZT; CAX; CAZ; CPE; GM; LVX; P/T; TO; T/S | aminoglycoside; fluoroquinolone; lactam; | CPE | lactam |
| | | other | | |
| 32 | AZT; CAX; CAZ; CPE; GM; LVX; P/T; TO; T/S | aminoglycoside; fluoroquinolone; lactam; | CPE | lactam |
| | | other | | |
| 33 | AZT; CAX; CAZ; CPE; GM; LVX; P/T; TO; T/S | aminoglycoside; fluoroquinolone; lactam; | CPE | lactam |
| | | other | | |
| 34 | AZT; CAX; CAZ; CPE; GM; LVX; P/T; TO; T/S | aminoglycoside; fluoroquinolone; lactam; | CPE | lactam |
| | | other | | |
| 35 | AZT; CAX; CAZ; CPE; GM; LVX; P/T; TO; T/S | aminoglycoside; fluoroquinolone; lactam; | CPE | lactam |
| | | other | | |
| 36 | AZT; CAX; CAZ; CPE; GM; LVX; P/T; TO; T/S | aminoglycoside; fluoroquinolone; lactam; | CPE | lactam |
| | | other | | |
| 37 | AZT; CAX; CAZ; CPE; GM; LVX; P/T; TO; T/S | aminoglycoside; fluoroquinolone; lactam; | CPE | lactam |
| | | other | | |
| 38 | CP; CPE; GM; LVX; TO; T/S | aminoglycoside; fluoroquinolone; lactam; | CPE | lactam |
| | | other | | |
| 39 | CP; CPE; GM; LVX; TO; T/S | aminoglycoside; fluoroquinolone; lactam; | CPE | lactam |
| | | other | | |
| 40 | AZT; CAX; CAZ; CFT; CRM; GM; P/T; TO; T/S | aminoglycoside; lactam; other | T/S | other |
| 41 | AZT; CAX; CAZ; CFT; CP; CPE; ETP; GM; LVX; P/T; | aminoglycoside; fluoroquinolone; lactam; | CPE | lactam |
| | TO; T/S | other | | |
| 42 | CP; CPE; GM; LVX; TO; T/S | aminoglycoside; fluoroquinolone; lactam; | CPE | lactam |
| | | other | | |
| 43 | AZT; CAX; CAZ; CPE; GM; LVX; P/T; TO; T/S | aminoglycoside; fluoroquinolone; lactam; | CPE | lactam |
| | | other | | |
| 44 | AZT; CAX; CAZ; CFT; CP; GM; LVX; P/T; TO; T/S | aminoglycoside; fluoroquinolone; lactam; | T/S | other |
| | | other | | |
| 45 | CAX; CAZ; CPE; GM; LVX; P/T; TO; T/S | aminoglycoside; fluoroquinolone; lactam; | CPE | lactam |
| | | other | | |
| 46 | CP; CPE; GM; LVX; TO; T/S | aminoglycoside; fluoroquinolone; lactam; | CPE | lactam |
| | | other | | |
| 47 | AZT; CAX; CAZ; CPE; GM; LVX; P/T; TO; T/S | aminoglycoside; fluoroquinolone; lactam; | CPE | lactam |
| | | other | | |
| 48 | AZT; CAX; CAZ; CPE; GM; LVX; P/T; TO; T/S | aminoglycoside; fluoroquinolone; lactam; | CPE | lactam |
| | | other | | |
| 49 | AZT; CAX; CAZ; CPE; GM; LVX; P/T; TO; T/S | aminoglycoside; fluoroquinolone; lactam; | CPE | lactam |
| | | other | | |
| 50 | AZT; CAX; CAZ; CPE; GM; LVX; P/T; TO; T/S | aminoglycoside; fluoroquinolone; lactam; | CPE | lactam |
| | | other | | |

TABLE 6c
Data for Enterobacter, particularly Enterobacter cloacae (continued)

| No. | num_aminoglycoside | num_fluoroquinolone | num_lactam | num_other |
|---|---|---|---|---|
| 1 | 2 | 2 | 8 | 1 |
| 2 | 2 | 2 | 7 | 1 |
| 3 | 2 | 2 | 7 | 1 |
| 4 | 2 | 2 | 7 | 1 |
| 5 | 2 | 2 | 7 | 1 |
| 6 | 2 | 2 | 7 | 1 |
| 7 | 2 | 2 | 5 | 1 |
| 8 | 2 | 2 | 5 | 1 |
| 9 | 2 | 2 | 6 | 1 |
| 10 | 2 | 2 | 7 | 1 |
| 11 | 2 | 2 | 5 | 1 |
| 12 | 2 | 2 | 6 | 1 |
| 13 | 2 | 2 | 5 | 1 |
| 14 | 2 | 2 | 8 | 1 |
| 15 | 2 | 2 | 8 | 1 |
| 16 | 2 | 2 | 8 | 1 |
| 17 | 2 | 2 | 7 | 1 |
| 18 | 2 | 2 | 8 | 1 |
| 19 | 2 | 2 | 8 | 1 |
| 20 | 2 | 2 | 7 | 1 |
| 21 | 2 | 2 | 6 | 1 |
| 22 | 2 | 2 | 5 | 1 |
| 23 | 2 | 2 | 6 | 1 |
| 24 | 2 | 2 | 6 | 1 |
| 25 | 2 | 2 | 7 | 1 |
| 26 | 2 | 2 | 6 | 1 |
| 27 | 2 | 2 | 6 | 1 |
| 28 | 2 | 1 | 6 | 0 |
| 29 | 2 | 1 | 6 | 1 |
| 30 | 2 | 2 | 5 | 1 |
| 31 | 2 | 1 | 5 | 1 |
| 32 | 2 | 1 | 5 | 1 |
| 33 | 2 | 1 | 5 | 1 |
| 34 | 2 | 1 | 5 | 1 |
| 35 | 2 | 1 | 5 | 1 |
| 36 | 2 | 1 | 5 | 1 |
| 37 | 2 | 1 | 5 | 1 |
| 38 | 2 | 2 | 1 | 1 |
| 39 | 2 | 2 | 1 | 1 |
| 40 | 2 | 0 | 6 | 1 |
| 41 | 2 | 2 | 7 | 1 |
| 42 | 2 | 2 | 1 | 1 |
| 43 | 2 | 1 | 5 | 1 |
| 44 | 2 | 2 | 5 | 1 |
| 45 | 2 | 1 | 4 | 1 |
| 46 | 2 | 2 | 1 | 1 |
| 47 | 2 | 1 | 5 | 1 |
| 48 | 2 | 1 | 5 | 1 |
| 49 | 2 | 1 | 5 | 1 |
| 50 | 2 | 1 | 5 | 1 |

TABLE 6d
Data for Enterobacter, particularly Enterobacter cloacae (continued)

| No. | AZT_pv_adj | CAX_pv_adj | CAZ_pv_adj | CFT_pv_adj | CP_pv_adj |
|---|---|---|---|---|---|
| 1 | 3.58E−10 | 7.39E−09 | 2.80E−11 | 3.62E−09 | 5.27E−08 |
| 2 | 4.06E−06 | 7.65E−06 | 8.35E−07 | 8.00E−07 | 3.72E−07 |
| 3 | 4.06E−06 | 7.65E−06 | 8.35E−07 | 8.00E−07 | 3.72E−07 |
| 4 | 4.06E−06 | 7.65E−06 | 8.35E−07 | 8.00E−07 | 3.72E−07 |
| 5 | 8.70E−07 | 8.75E−06 | 5.21E−07 | 5.80E−07 | 1.18E−06 |
| 6 | 1.92E−12 | 2.08E−13 | 5.48E−12 | 1.92E−12 | 4.74E−09 |
| 7 | 2.20E−05 | 4.05E−05 | 4.94E−06 | 4.85E−06 | 7.75E−07 |
| 8 | 2.20E−05 | 4.05E−05 | 4.94E−06 | 4.85E−06 | 7.75E−07 |
| 9 | 6.62E−06 | 1.21E−05 | 1.34E−06 | 1.27E−06 | 3.72E−07 |
| 10 | 3.70E−06 | 6.73E−06 | 7.48E−07 | 7.08E−07 | 2.65E−06 |
| 11 | 3.46E−05 | 6.07E−05 | 7.99E−06 | 7.84E−06 | 7.16E−07 |
| 12 | 6.62E−06 | 1.21E−05 | 1.34E−06 | 1.27E−06 | 3.14E−06 |
| 13 | 2.20E−05 | 4.05E−05 | 4.94E−06 | 4.85E−06 | 7.75E−07 |
| 14 | 2.41E−09 | 3.66E−10 | 2.89E−10 | 1.33E−09 | 1.31E−11 |
| 15 | 6.79E−08 | 4.49E−08 | 1.13E−08 | 3.99E−08 | 4.19E−12 |
| 16 | 3.51E−09 | 1.67E−09 | 8.09E−10 | 8.35E−10 | 5.65E−12 |
| 17 | 3.11E−11 | 6.47E−11 | 7.01E−12 | 5.47E−11 | 1.53E−06 |
| 18 | 4.36E−12 | 6.39E−12 | 5.85E−13 | 5.48E−12 | 5.70E−07 |
| 19 | 6.79E−08 | 4.49E−08 | 1.13E−08 | 3.99E−08 | 3.61E−11 |
| 20 | 5.39E−09 | 1.23E−08 | 5.84E−09 | 6.70E−09 | 5.19E−07 |
| 21 | 3.36E−07 | 5.25E−07 | 5.19E−07 | 3.74E−06 | 3.91E−05 |
| 22 | 4.53E−07 | 8.07E−07 | 7.17E−07 | 6.01E−06 | 7.16E−05 |
| 23 | 1.62E−07 | 3.02E−07 | 2.83E−08 | 5.65E−08 | 9.46E−05 |
| 24 | 8.58E−09 | 1.75E−08 | 5.92E−08 | 2.01E−07 | 7.82E−07 |
| 25 | 5.28E−06 | 5.19E−07 | 1.32E−06 | 7.50E−07 | 1.27E−05 |
| 26 | 3.71E−05 | 2.06E−06 | 1.56E−05 | 9.73E−06 | 1.34E−04 |
| 27 | 2.41E−04 | 8.91E−05 | 8.91E−05 | 4.87E−04 | 5.72E−04 |
| 28 | 2.12E−03 | 7.75E−04 | 1.61E−03 | 5.63E−03 | 2.33E−02 |
| 29 | 3.60E−03 | 1.44E−03 | 1.46E−03 | 6.84E−03 | 1.58E−02 |
| 30 | 1.63E−03 | 1.30E−02 | 1.26E−03 | 9.88E−03 | 1.03E−03 |
| 31 | 8.73E−03 | 1.90E−03 | 3.60E−03 | 1.54E−02 | 2.18E−02 |
| 32 | 8.73E−03 | 1.90E−03 | 3.60E−03 | 1.54E−02 | 2.18E−02 |
| 33 | 8.73E−03 | 1.90E−03 | 3.60E−03 | 1.54E−02 | 2.18E−02 |
| 34 | 8.73E−03 | 1.90E−03 | 3.60E−03 | 1.54E−02 | 2.18E−02 |
| 35 | 8.73E−03 | 1.90E−03 | 3.60E−03 | 1.54E−02 | 2.18E−02 |
| 36 | 8.73E−03 | 1.90E−03 | 3.60E−03 | 1.54E−02 | 2.18E−02 |
| 37 | 9.06E−03 | 2.62E−03 | 4.92E−03 | 1.99E−02 | 2.22E−02 |
| 38 | 3.58E−02 | 1.04E−02 | 1.75E−02 | 5.31E−02 | 2.20E−05 |
| 39 | 5.33E−02 | 1.78E−02 | 2.85E−02 | 7.63E−02 | 6.85E−05 |
| 40 | 8.52E−05 | 3.90E−05 | 6.01E−06 | 2.49E−05 | 1.29E−02 |
| 41 | 4.11E−06 | 7.41E−04 | 4.26E−05 | 1.08E−03 | 6.16E−05 |
| 42 | 4.35E−02 | 1.36E−02 | 2.23E−02 | 6.48E−02 | 2.37E−05 |
| 43 | 8.79E−03 | 2.56E−03 | 4.80E−03 | 1.96E−02 | 2.95E−02 |
| 44 | 5.44E−03 | 1.03E−03 | 6.23E−04 | 1.01E−03 | 1.45E−04 |
| 45 | 1.49E−02 | 4.69E−03 | 8.56E−03 | 3.09E−02 | 3.86E−02 |
| 46 | 2.22E−01 | 6.69E−02 | 1.35E−01 | 3.01E−01 | 6.26E−04 |
| 47 | 8.87E−03 | 1.93E−03 | 3.69E−03 | 1.56E−02 | 3.05E−02 |
| 48 | 8.87E−03 | 1.93E−03 | 3.69E−03 | 1.56E−02 | 3.05E−02 |
| 49 | 8.87E−03 | 1.93E−03 | 3.69E−03 | 1.56E−02 | 3.05E−02 |
| 50 | 8.87E−03 | 1.93E−03 | 3.69E−03 | 1.56E−02 | 3.05E−02 |

TABLE 6e
Data for Enterobacter, particularly
Enterobacter cloacae (continued)

| No. | CPE_pv_adj | CRM_pv_adj | ETP_pv_adj | GM_pv_adj |
|---|---|---|---|---|
| 1 | 2.76E−09 | 1.20E−03 | 1.29E−04 | 3.48E−19 |
| 2 | 2.95E−05 | 2.74E−03 | 5.44E−02 | 5.95E−17 |
| 3 | 2.95E−05 | 2.74E−03 | 5.44E−02 | 5.95E−17 |
| 4 | 2.95E−05 | 2.74E−03 | 5.44E−02 | 5.95E−17 |
| 5 | 5.40E−05 | 2.87E−03 | 9.20E−02 | 6.93E−16 |
| 6 | 3.27E−08 | 3.20E−02 | 6.76E−03 | 1.07E−19 |
| 7 | 6.14E−05 | 1.06E−02 | 5.77E−02 | 2.51E−16 |
| 8 | 6.14E−05 | 1.06E−02 | 5.77E−02 | 2.51E−16 |
| 9 | 5.81E−05 | 2.77E−03 | 7.62E−02 | 4.00E−16 |
| 10 | 5.02E−05 | 3.99E−03 | 3.88E−02 | 2.55E−15 |
| 11 | 5.72E−05 | 1.10E−02 | 5.64E−02 | 1.59E−16 |
| 12 | 5.81E−05 | 6.94E−03 | 4.04E−02 | 4.15E−15 |
| 13 | 1.93E−05 | 1.06E−02 | 5.77E−02 | 2.51E−16 |
| 14 | 5.33E−09 | 5.60E−04 | 8.61E−04 | 9.13E−23 |
| 15 | 1.62E−07 | 1.89E−03 | 7.13E−04 | 4.13E−22 |
| 16 | 1.55E−08 | 1.01E−03 | 1.04E−03 | 4.49E−22 |
| 17 | 5.31E−08 | 9.22E−03 | 1.04E−02 | 1.03E−21 |
| 18 | 1.37E−08 | 1.77E−03 | 7.24E−03 | 1.52E−21 |
| 19 | 3.31E−08 | 1.89E−03 | 6.74E−03 | 5.26E−21 |
| 20 | 5.79E−05 | 2.43E−03 | 5.85E−02 | 1.07E−14 |
| 21 | 8.87E−03 | 1.21E−02 | 2.44E−01 | 2.97E−08 |
| 22 | 1.76E−02 | 1.63E−02 | 2.41E−01 | 7.76E−08 |
| 23 | 2.82E−06 | 7.01E−02 | 5.22E−02 | 2.31E−14 |
| 24 | 3.31E−04 | 2.06E−02 | 1.04E−02 | 3.85E−13 |
| 25 | 1.65E−04 | 1.14E−01 | 1.77E−03 | 5.27E−08 |
| 26 | 1.92E−04 | 5.27E−02 | 1.45E−03 | 1.24E−08 |
| 27 | 1.84E−09 | 3.12E−02 | 9.47E−02 | 3.86E−04 |
| 28 | 3.38E−09 | 2.11E−01 | 1.66E−01 | 6.74E−04 |
| 29 | 5.84E−09 | 6.96E−02 | 2.65E−01 | 9.71E−04 |
| 30 | 8.81E−09 | 1.75E−01 | 1.27E−01 | 2.57E−04 |
| 31 | 1.29E−08 | 8.58E−02 | 3.28E−01 | 1.48E−03 |
| 32 | 1.29E−08 | 8.58E−02 | 3.28E−01 | 1.48E−03 |
| 33 | 1.29E−08 | 8.58E−02 | 3.28E−01 | 1.48E−03 |
| 34 | 1.29E−08 | 8.58E−02 | 3.28E−01 | 1.48E−03 |
| 35 | 1.29E−08 | 8.58E−02 | 3.28E−01 | 1.48E−03 |
| 36 | 1.29E−08 | 8.58E−02 | 3.28E−01 | 1.48E−03 |
| 37 | 2.23E−08 | 8.52E−02 | 3.28E−01 | 1.51E−03 |
| 38 | 2.39E−08 | 4.38E−01 | 5.64E−02 | 4.16E−03 |
| 39 | 2.39E−08 | 4.38E−01 | 5.64E−02 | 4.16E−03 |
| 40 | 1.69E−01 | 6.82E−04 | 7.60E−01 | 2.98E−04 |
| 41 | 3.02E−08 | 3.74E−02 | 3.81E−04 | 1.38E−04 |
| 42 | 4.40E−08 | 3.77E−01 | 5.72E−02 | 4.38E−03 |
| 43 | 4.44E−08 | 6.75E−02 | 3.31E−01 | 2.17E−03 |
| 44 | 2.31E−02 | 4.84E−02 | 6.10E−02 | 1.15E−06 |
| 45 | 5.15E−08 | 8.55E−02 | 2.60E−01 | 3.19E−03 |
| 46 | 5.15E−08 | 7.80E−02 | 4.73E−01 | 1.13E−03 |
| 47 | 5.31E−08 | 1.08E−01 | 2.67E−01 | 2.37E−03 |
| 48 | 5.31E−08 | 1.08E−01 | 2.67E−01 | 2.37E−03 |
| 49 | 5.31E−08 | 1.08E−01 | 2.67E−01 | 2.37E−03 |
| 50 | 5.31E−08 | 1.08E−01 | 2.67E−01 | 2.37E−03 |

TABLE 6f
Data for Enterobacter, particularly
Enterobacter cloacae (continued)

| No. | LVX_pv_adj | P/T_pv_adj | TO_pv_adj | T/S_pv_adj |
|---|---|---|---|---|
| 1 | 8.81E−09 | 1.52E−04 | 2.06E−19 | 5.49E−27 |
| 2 | 2.74E−08 | 6.34E−03 | 2.69E−20 | 5.83E−25 |
| 3 | 2.74E−08 | 6.34E−03 | 2.69E−20 | 5.83E−25 |
| 4 | 2.74E−08 | 6.34E−03 | 2.69E−20 | 5.83E−25 |
| 5 | 5.07E−07 | 6.25E−03 | 9.93E−19 | 1.25E−24 |
| 6 | 1.21E−11 | 4.05E−05 | 5.70E−21 | 2.32E−24 |
| 7 | 5.15E−08 | 1.58E−02 | 1.59E−19 | 2.46E−24 |
| 8 | 5.15E−08 | 1.58E−02 | 1.59E−19 | 2.46E−24 |
| 9 | 7.06E−08 | 1.10E−02 | 1.73E−19 | 2.46E−24 |
| 10 | 3.37E−07 | 8.08E−03 | 1.06E−18 | 1.59E−23 |
| 11 | 4.09E−08 | 2.07E−02 | 9.23E−20 | 1.59E−23 |
| 12 | 3.72E−07 | 1.10E−02 | 1.95E−18 | 2.83E−23 |
| 13 | 5.15E−08 | 1.58E−02 | 1.59E−19 | 2.83E−23 |
| 14 | 1.31E−11 | 2.13E−07 | 5.43E−22 | 1.85E−22 |
| 15 | 4.19E−12 | 8.40E−07 | 8.04E−22 | 2.17E−22 |
| 16 | 5.65E−12 | 5.70E−07 | 2.63E−21 | 1.03E−21 |
| 17 | 1.00E−08 | 4.60E−04 | 1.44E−21 | 4.11E−18 |
| 18 | 2.49E−09 | 1.68E−04 | 1.49E−21 | 4.99E−18 |
| 19 | 4.19E−12 | 8.40E−07 | 7.92E−21 | 2.34E−21 |
| 20 | 6.44E−09 | 2.28E−04 | 7.17E−16 | 1.02E−15 |
| 21 | 8.28E−05 | 6.44E−04 | 3.80E−10 | 1.05E−15 |
| 22 | 1.82E−04 | 9.24E−04 | 1.96E−09 | 4.35E−15 |
| 23 | 1.55E−05 | 1.40E−04 | 3.69E−14 | 6.50E−11 |
| 24 | 7.82E−07 | 1.61E−04 | 8.30E−12 | 1.21E−11 |
| 25 | 3.34E−06 | 3.34E−03 | 5.47E−11 | 4.91E−08 |
| 26 | 6.31E−06 | 1.70E−02 | 9.07E−10 | 5.12E−08 |
| 27 | 2.79E−05 | 2.12E−03 | 1.68E−04 | 4.44E−04 |
| 28 | 2.35E−03 | 8.21E−03 | 2.41E−03 | 3.78E−02 |
| 29 | 1.41E−03 | 2.25E−03 | 9.05E−04 | 1.74E−03 |
| 30 | 1.97E−05 | 1.77E−03 | 8.04E−05 | 5.50E−04 |
| 31 | 2.08E−03 | 5.50E−03 | 1.36E−03 | 3.40E−03 |
| 32 | 2.08E−03 | 5.50E−03 | 1.36E−03 | 3.40E−03 |
| 33 | 2.08E−03 | 5.50E−03 | 1.36E−03 | 3.40E−03 |
| 34 | 2.08E−03 | 5.50E−03 | 1.36E−03 | 3.40E−03 |
| 35 | 2.08E−03 | 5.50E−03 | 1.36E−03 | 3.40E−03 |
| 36 | 2.08E−03 | 5.50E−03 | 1.36E−03 | 3.40E−03 |
| 37 | 2.10E−03 | 7.39E−03 | 1.42E−03 | 3.42E−03 |
| 38 | 1.40E−06 | 4.00E−02 | 2.39E−03 | 8.45E−04 |
| 39 | 5.74E−06 | 7.03E−02 | 2.39E−03 | 8.45E−04 |
| 40 | 1.29E−02 | 8.60E−03 | 1.87E−05 | 2.77E−08 |
| 41 | 6.73E−06 | 8.68E−06 | 4.49E−05 | 2.53E−04 |
| 42 | 1.52E−06 | 4.87E−02 | 2.44E−03 | 8.72E−04 |
| 43 | 3.07E−03 | 5.47E−03 | 1.99E−03 | 2.25E−03 |
| 44 | 1.45E−04 | 6.88E−03 | 4.69E−06 | 5.12E−08 |
| 45 | 3.22E−03 | 9.52E−03 | 2.86E−03 | 3.22E−03 |
| 46 | 6.11E−05 | 1.82E−01 | 2.53E−04 | 3.05E−04 |
| 47 | 3.31E−03 | 5.73E−03 | 2.11E−03 | 5.16E−03 |
| 48 | 3.31E−03 | 5.73E−03 | 2.11E−03 | 5.16E−03 |
| 49 | 3.31E−03 | 5.73E−03 | 2.11E−03 | 5.16E−03 |
| 50 | 3.31E−03 | 5.73E−03 | 2.11E−03 | 5.16E−03 |

TABLE 6g
Data for Enterobacter, particularly Enterobacter cloacae (continued)

| No. | Locus.Tag | Gene.Symbol | GenBank.Accession | Chromosome |
|---|---|---|---|---|
| 1 | ETAE_p041 | folP | YP_003297635 | plasmid pEIB202 |
| 2 | ECL_A197 | | YP_003602692 | plasmid pECL_A |
| 3 | APECO1_O1R60 | terY1 | YP_001481413 | plasmid pAPEC-O1-R |
| 4 | APECO1_O1R58 | terY2 | YP_001481411 | plasmid pAPEC-O1-R |
| 5 | ECL_A215 | | YP_003602710 | plasmid pECL_A |
| 6 | ECL_A245 | | YP_003602738 | plasmid pECL_A |
| 7 | ECL_A211 | | YP_003602706 | plasmid pECL_A |
| 8 | ECL_A213 | | YP_003602708 | plasmid pECL_A |
| 9 | CSAG_04328 | | ZP_04559059 | |
| 10 | CSAG_04340 | | ZP_04559071 | |
| 11 | APECO1_O1R72 | terD | YP_001481425 | plasmid pAPEC-O1-R |
| 12 | ESA_01791 | | YP_001437881 | |
| 13 | ECL_A210 | | YP_003602705 | plasmid pECL_A |
| 14 | HMPREF9551_04611 | | ZP_07191964 | |
| 15 | ECL_03822 | merP | YP_003614305 | |
| 16 | Salmoentericaenterica_010100000105 | | ZP_02702049 | |
| 17 | S2707 | parA | NP_838057 | |
| 18 | ECL_03815 | | YP_003614298 | |
| 19 | HMPREF9538_01300 | | ZP_08303641 | |
| 20 | ECL_00479 | | YP_003610994 | |
| 21 | SFxv_17317 | | ADA73942 | |
| 22 | EcDH1_2078 | | ACX39734 | |
| 23 | HMPREF9347_01086 | | ZP_07208640 | |
| 24 | HMPREF9530_03894 | | ZP_07153755 | |
| 25 | ECL_00391 | | YP_003610906 | |
| 26 | EcolE2_01002790 | | ZP_00728858 | |
| 27 | ENC_13410 | | CBK85143 | |
| 28 | ENC_03420 | | CBK84384 | |
| 29 | ENC_31170 | | CBK86512 | |
| 30 | HMPREF9086_2435 | | ZP_08498173 | |
| 31 | ENC_11900 | | CBK85029 | |
| 32 | ENC_02650 | | CBK84321 | |
| 33 | ENC_02700 | | CBK84325 | |
| 34 | HMPREF9086_4455 | amsH2 | ZP_08500191 | |
| 35 | HMPREF9086_4454 | yccY2 | ZP_08500190 | |
| 36 | HMPREF9086_2100 | yjdF | ZP_08497838 | |
| 37 | HMPREF9086_4460 | ligA2 | ZP_08500196 | |
| 38 | ENC_28850 | | CBK86326 | |
| 39 | HMPREF9086_3524 | gatAX | ZP_08499260 | |
| 40 | Entcl_2219 | | YP_003941757 | |
| 41 | Entcl_2207 | | YP_003941745 | |
| 42 | ENTCAN_03923 | | ZP_03284103 | |
| 43 | ECL_01132 | | YP_003611642 | |
| 44 | CSAG_04209 | | ZP_04558940 | |
| 45 | Entcl_0777 | | YP_003940336 | |
| 46 | HMPREF9086_0855 | | ZP_08496597 | |
| 47 | ENC_21030 | | CBK85719 | |
| 48 | ENC_20990 | | CBK85716 | |
| 49 | ENC_21000 | | CBK85717 | |
| 50 | ENC_21040 | | CBK85720 | |

TABLE 6h
Data for Enterobacter, particularly
Enterobacter cloacae (continued)

| No. | Start.Coord | End.Coord | Strand | Scaffold.ID |
|---|---|---|---|---|
| 1 | 32101 | 32916 | − | 646311959 |
| 2 | 142295 | 142936 | − | 646564617 |
| 3 | 68792 | 69511 | − | 640753093 |
| 4 | 67490 | 68131 | − | 640753093 |
| 5 | 156077 | 157318 | + | 646564617 |
| 6 | 176747 | 177670 | − | 646564617 |
| 7 | 153338 | 154378 | + | 646564617 |
| 8 | 155073 | 155648 | + | 646564617 |
| 9 | 377133 | 378173 | − | 646206736 |
| 10 | 388284 | 389441 | + | 646206736 |
| 11 | 80263 | 80841 | + | 640753093 |
| 12 | 1731242 | 1732198 | − | 640753067 |
| 13 | 152860 | 153315 | + | 646564617 |
| 14 | 97 | 531 | − | 648293053 |
| 15 | 3908948 | 3909223 | + | 646564624 |
| 16 | 16271 | 16636 | − | 641778223 |
| 17 | 2602155 | 2602892 | − | 637000219 |
| 18 | 3903410 | 3903712 | + | 646564624 |
| 19 | 6404 | 6640 | + | 651333085 |
| 20 | 482851 | 484329 | − | 646564624 |
| 21 | 1636971 | 1637258 | − | 646862363 |
| 22 | 2232606 | 2232845 | + | 646862383 |
| 23 | 2726 | 5692 | − | 648294279 |
| 24 | 1918 | 3231 | − | 648290583 |
| 25 | 409807 | 410283 | + | 646564624 |
| 26 | 14395 | 15375 | + | 638358747 |
| 27 | 1397946 | 1398188 | − | 650378029 |
| 28 | 360699 | 361856 | + | 650378029 |
| 29 | 3151035 | 3151685 | − | 650378029 |
| 30 | 192614 | 193519 | + | 651335147 |
| 31 | 1250898 | 1252163 | + | 650378029 |
| 32 | 274155 | 276332 | − | 650378029 |
| 33 | 280790 | 281458 | − | 650378029 |
| 34 | 111547 | 112680 | − | 651335177 |
| 35 | 111116 | 111559 | − | 651335177 |
| 36 | 33364 | 34074 | + | 651335138 |
| 37 | 117314 | 119053 | − | 651335177 |
| 38 | 2904606 | 2905577 | − | 650378029 |
| 39 | 8440 | 9756 | − | 651335166 |
| 40 | 2349542 | 2349967 | + | 649633020 |
| 41 | 2334593 | 2335594 | − | 649633020 |
| 42 | 28272 | 29567 | − | 643006662 |
| 43 | 1168855 | 1169388 | + | 646564624 |
| 44 | 254005 | 255187 | − | 646206736 |
| 45 | 812618 | 813094 | + | 649633020 |
| 46 | 13947 | 15560 | − | 651335119 |
| 47 | 2148896 | 2151391 | + | 650378029 |
| 48 | 2145171 | 2145458 | + | 650378029 |
| 49 | 2145486 | 2146736 | + | 650378029 |
| 50 | 2151395 | 2152267 | − | 650378029 |

### Example 4:

### Escherichia, particularly E. coli

The procedure was carried out as in Example 1, except that the following microorganisms were used:

Bacterial Strains

The inventors selected 1144 E.coli strains from the microbiology strain collection at Siemens Healthcare Diagnostics (West Sacramento, Calif.) for susceptibility testing and whole genome sequencing.

From MetaRef, 318 centroids of E.coli were used as reference sequences.

The results for E.coli are shown in Tables 7 (corresponding to Table 1) and 8 (corresponding to Table 2).

TABLE 7a
Data for Escherichia, particularly E. coli

| | Scaffold.External. | | |
|---|---|---|---|
| No. | Accession | Scaffold.Name | best_pv |
| 1 | NC_011083 | Salmonella enterica subsp. enterica serovar Heidelberg str. | 3.5041948925527e−207 |
| | | SL476: NC_011083 | |
| 2 | NZ_AAJT01000012 | Escherichia coli B7A, unfinished sequence: NZ_AAJT01000012 | 1.62252621621877e−119 |
| 3 | NZ_ADWV01000045 | Escherichia coli MS 107-1 E_coliMS107-1-1.0.1_Cont44.1: | 6.24750842070913e−97 |
| | | NZ_ADWV01000045 | |
| 4 | NC_009651 | Klebsiella pneumoniae subsp. pneumoniae MGH 78578 plasmid | 8.77735582031733e−96 |
| | | pKPN5: NC_009651 | |
| 5 | NZ_ADUB01000261 | Escherichia coli MS 175-1 E_coli175-1-1.0_Cont542.3: | 1.23709746311649e−80 |
| | | NZ_ADUB01000261 | |
| 6 | NC_011092 | Salmonella enterica subsp. enterica serovar Schwarzengrund | 2.41688550175243e−75 |
| | | str. CVM19633 plasmid pCVM19633_110: NC_011092 | |
| 7 | NZ_ADWV01000056 | Escherichia coli MS 107-1 E_coliMS107-1-1.0.1_Cont55.1: | 7.68091736117399e−72 |
| | | NZ_ADWV01000056 | |
| 8 | NZ_AAJX01000089 | Escherichia coli B171, unfinished sequence: NZ_AAJX01000089 | 1.57772112100981e−71 |
| 9 | NC_011743 | Escherichia fergusonii ATCC 35469 plasmid pEFER: NC_011743 | 1.0118246517332e−69 |
| 10 | NC_013509 | Edwardsiella tarda EIB202 plasmid pEIB202: NC_013509 | 1.23255842888535e−68 |
| 11 | NZ_GG749218 | Escherichia coli FVEC1412 genomic scaffold supercont1.13: | 6.77313303746994e−68 |
| | | NZ_GG749218 | |
| 12 | NC_013509 | Edwardsiella tarda EIB202 plasmid pEIB202: NC_013509 | 1.94619564613577e−64 |
| 13 | NZ_GG749218 | Escherichia coli FVEC1412 genomic scaffold supercont1.13: | 1.86737834552034e−63 |
| | | NZ_GG749218 | |
| 14 | NC_013717 | Citrobacter rodentium ICC168 plasmid pCROD1: NC_013717 | 7.42780365363135e−62 |
| 15 | NC_013717 | Citrobacter rodentium ICC168 plasmid pCROD1: NC_013717 | 3.07751376659308e−61 |
| 16 | NZ_ADWS01000089 | Escherichia coli MS 145-7 E_coliMS145-7-1.0.1_Cont88.1: | 1.7351795845908e−60 |
| | | NZ_ADWS01000089 | |
| 17 | NC_011751 | Escherichia coli UMN026: NC_011751 | 2.44317906034667e−60 |
| 18 | NC_011751 | Escherichia coli UMN026: NC_011751 | 6.73588199342071e−60 |
| 19 | NZ_ADWV01000056 | Escherichia coli MS 107-1 E_coliMS107-1-1.0.1_Cont55.1: | 2.62943019143703e−59 |
| | | NZ_ADWV01000056 | |
| 20 | NC_010488 | Escherichia coli SECEC SMS-3-5 plasmid pSMS35_130: NC_010488 | 3.46702381924878e−47 |
| 21 | NZ_ADTM01000311 | Escherichia coli MS 182-1 E_coli182-1-1.0_Cont460.3: | 4.65308035729244e−46 |
| | | NZ_ADTM01000311 | |
| 22 | NZ_ADWS01000095 | Escherichia coli MS 145-7 E_coliMS145-7-1.0.1_Cont94.1: | 7.71451120848483e−46 |
| | | NZ_ADWS01000095 | |
| 23 | NZ_ADTM01000311 | Escherichia coli MS 182-1 E_coli182-1-1.0_Cont460.3: | 1.08551755499227e−44 |
| | | NZ_ADTM01000311 | |
| 24 | NC_009650 | Klebsiella pneumoniae subsp. pneumoniae MGH 78578 plasmid | 4.89761264283562e−44 |
| | | pKPN4: NC_009650 | |
| 25 | NZ_ADWV01000017 | Escherichia coli MS 107-1 E_coliMS107-1-1.0.1_Cont16.1: | 6.97693050178087e−44 |
| | | NZ_ADWV01000017 | |
| 26 | NC_009650 | Klebsiella pneumoniae subsp. pneumoniae MGH 78578 plasmid | 9.68294892967391e−44 |
| | | pKPN4: NC_009650 | |
| 27 | NZ_GG665811 | Escherichia sp. 1_1_43 genomic scaffold supercont1.1: | 3.5843867496487e−43 |
| | | NZ_GG665811 | |
| 28 | NZ_ADTL01000051 | Escherichia coli MS 115-1 E_coli115-1-1.0_Cont158.2: | 3.5843867496487e−43 |
| | | NZ_ADTL01000051 | |
| 29 | AP011957 | Salmonella enterica subsp. enterica serovar Typhimurium str. | 5.53805522527039e−43 |
| | | T000240 DNA: AP011957 | |
| 30 | NC_011282 | Klebsiella pneumoniae 342 plasmid pKP187: NC_011282 | 2.71226651272988e−42 |
| 31 | NZ_ABHR01000011 | Escherichia coli O157:H7 str. EC4401, unfinished sequence: | 8.45082785112866e−42 |
| | | NZ_ABHR01000011 | |
| 32 | NC_010488 | Escherichia coli SECEC SMS-3-5 plasmid pSMS35_130: NC_010488 | 1.02749291231853e−41 |
| 33 | NZ_AFBO01000013 | Klebsiella sp. MS 92-3 K_spMS92-3-l.0_Cont24.1: | 1.46909169086738e−41 |
| | | NZ_AFBO01000013 | |
| 34 | CP002797 | Escherichia coli NA114: CP002797 | 2.43588122712712e−41 |
| 35 | NZ_ABHK01000002 | Escherichia coli O157:H7 str. EC4206, unfinished sequence: | 2.60702932643171e−41 |
| | | NZ_ABHK01000002 | |
| 36 | NZ_GG657366 | Citrobacter sp. 30_2 genomic scaffold supercont1.1: | 2.60702932643171e−41 |
| | | NZ_GG657366 | |
| 37 | NC_011353 | Escherichia coli O157:H7 str. EC4115: NC_011353 | 2.60702932643171e−41 |
| 38 | NZ_ABWL01000012 | Citrobacter youngae ATCC 29220 C_sp-1.0_Cont2.3: | 3.21133372679738e−41 |
| | | NZ_ABWL01000012 | |
| 39 | AP009378 | Escherichia coli SE15 DNA: AP009378 | 3.68640118720819e−41 |
| 40 | NZ_ACXN01000074 | Escherichia coli O157:H7 str. FRIK966: NZ_ACXN01000074 | 5.75695041652081e−41 |
| 41 | NC_007384 | Shigella sonnei Ss046: NC_007384 | 6.75951367822551e−41 |
| 42 | NZ_ABWL01000012 | Citrobacter youngae ATCC 29220 C_sp-1.0_Cont2.3: | 8.12681193093067e−41 |
| | | NZ_ABWL01000012 | |
| 43 | NZ_ADTL01000083 | Escherichia coli MS 115-1 E_coli115-1-1.0_Cont264.1: | 8.12681193093067e−41 |
| | | NZ_ADTL01000083 | |
| 44 | AC_000091 | Escherichia coli W3110 DNA: AC_000091 | 8.12681193093067e−41 |
| 45 | NZ_ADUD01000537 | Escherichia coli MS 196-1 E_colil96-1-1.0_Cont1482.1: | 1.06521634399457e−40 |
| | | NZ_ADUD01000537 | |
| 46 | NC_011083 | Salmonella enterica subsp. enterica serovar Heidelberg str. | 1.84998224240006e−40 |
| | | SL476: NC_011083 | |
| 47 | CP001383 | Shigella flexneri 2002017: CP001383 | 1.89942261631873e−40 |
| 48 | NZ_ADTR01000134 | Escherichia coli MS 21-1 E_coli21-1-1.0_Cont296.1: | 3.35939290103889e−40 |
| | | NZ_ADTR01000134 | |
| 49 | NC_009650 | Klebsiella pneumoniae subsp. pneumoniae MGH 78578 plasmid | 3.76989142366575e−40 |
| | | pKPN4: NC_009650 | |
| 50 | NC_004851 | Shigella flexneri 2a str. 301 plasmid pCP301: NC_004851 | 8.68449218790606e−39 |

TABLE 7b
Data for Escherichia, particularly E. coli (continued)

| No. | sign_phenos | sign_phenos_class | best_pheno | best_pheno_class |
|---|---|---|---|---|
| 1 | AM; A/S; AUG; CAX; CP; CRM; GM; | aminoglycoside; fluoroquinolone; lactam; other | AM | lactam |
| | LVX; TO; T/S | | | |
| 2 | AM; A/S; AUG; AZT; CAX; CFT; CP; | aminoglycoside; fluoroquinolone; lactam; other | T/S | other |
| | CRM; GM; LVX; TO; T/S | | | |
| 3 | AM; A/S; AUG; AZT; CAX; CFT; CP; | aminoglycoside; fluoroquinolone; lactam; other | T/S | other |
| | CRM; GM; LVX; TO; T/S | | | |
| 4 | AM; A/S; AUG; AZT; CAX; CFT; CP; | aminoglycoside; fluoroquinolone; lactam; other | T/S | other |
| | CRM; GM; LVX; TO; T/S | | | |
| 5 | AM; A/S; AUG; CRM | lactam | AM | lactam |
| 6 | AM; A/S; AUG | lactam | AM | lactam |
| 7 | AM; A/S; AUG; AZT; CAX; CFT; CP; | aminoglycoside; fluoroquinolone; lactam; other | T/S | other |
| | CRM; GM; LVX; TO; T/S | | | |
| 8 | AM; A/S; AUG; AZT; CAX; CFT; CP; | aminoglycoside; fluoroquinolone; lactam; other | T/S | other |
| | CRM; GM; LVX; TO; T/S | | | |
| 9 | AM; A/S; AUG; AZT; CAX; CFT; CP; | aminoglycoside; fluoroquinolone; lactam; other | T/S | other |
| | CRM; GM; LVX; TO; T/S | | | |
| 10 | AM; A/S; AUG; AZT; CAX; CFT; CP; | aminoglycoside; fluoroquinolone; lactam; other | T/S | other |
| | CRM; GM; LVX; TO; T/S | | | |
| 11 | AM; A/S; AUG; AZT; CAX; CFT; CP; | aminoglycoside; fluoroquinolone; lactam; other | T/S | other |
| | CRM; GM; LVX; TO; T/S | | | |
| 12 | AM; A/S; AUG; AZT; CAX; CFT; CP; | aminoglycoside; fluoroquinolone; lactam; other | T/S | other |
| | CRM; GM; LVX; TO; T/S | | | |
| 13 | AM; A/S; AUG; AZT; CAX; CFT; CP; | aminoglycoside; fluoroquinolone; lactam; other | T/S | other |
| | CRM; GM; LVX; TO; T/S | | | |
| 14 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 15 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 16 | AM; A/S; AUG; AZT; CAX; CFT; CP; | aminoglycoside; fluoroquinolone; lactam; other | T/S | other |
| | CRM; GM; LVX; TO; T/S | | | |
| 17 | AM; A/S; AUG; AZT; CAX; CFT; CP; | aminoglycoside; fluoroquinolone; lactam; other | T/S | other |
| | CRM; GM; LVX; TO; T/S | | | |
| 18 | AM; A/S; AUG; AZT; CAX; CFT; CP; | aminoglycoside; fluoroquinolone; lactam; other | T/S | other |
| | CRM; GM; LVX; TO; T/S | | | |
| 19 | AM; A/S; AUG; AZT; CAX; CFT; CP; | aminoglycoside; fluoroquinolone; lactam; other | T/S | other |
| | CRM; GM; LVX; TO; T/S | | | |
| 20 | AM; A/S; AUG; AZT; CAX; CFT; CP; | aminoglycoside; fluoroquinolone; lactam; other | T/S | other |
| | CRM; GM; LVX; TO; T/S | | | |
| 21 | AM; A/S; AUG; AZT; CAX; CFT; CP; | aminoglycoside; fluoroquinolone; lactam; other | CAX | lactam |
| | CRM; GM; LVX; TO; T/S | | | |
| 22 | AM; A/S; AUG; AZT; CAX; CFT; CP; | aminoglycoside; fluoroquinolone; lactam; other | T/S | other |
| | CRM; GM; LVX; TO; T/S | | | |
| 23 | AM; A/S; AUG; AZT; CAX; CFT; CP; | aminoglycoside; fluoroquinolone; lactam; other | AM | lactam |
| | CRM; GM; LVX; TO; T/S | | | |
| 24 | AM; A/S; AUG; CAX; CFT; CP; CRM; | aminoglycoside; fluoroquinolone; lactam; other | T/S | other |
| | GM; LVX; TO; T/S | | | |
| 25 | AM; A/S; AUG; AZT; CAX; CFT; CP; | aminoglycoside; fluoroquinolone; lactam; other | T/S | other |
| | CRM; GM; LVX; T/S | | | |
| 26 | AM; A/S; AUG; AZT; CAX; CFT; CP; | aminoglycoside; fluoroquinolone; lactam; other | T/S | other |
| | CRM; GM; TO; T/S | | | |
| 27 | AM; AUG; AZT; CAX; CFT; CP; CRM; | aminoglycoside; fluoroquinolone; lactam; other | LVX | fluoroquinolone |
| | GM; LVX; TO; T/S | | | |
| 28 | AM; AUG; AZT; CAX; CFT; CP; CRM; | aminoglycoside; fluoroquinolone; lactam; other | LVX | fluoroquinolone |
| | GM; LVX; TO; T/S | | | |
| 29 | AM; A/S; AUG; CAX; CFT; CP; CRM; | aminoglycoside; fluoroquinolone; lactam; other | T/S | other |
| | GM; LVX; T/S | | | |
| 30 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 31 | AM; A/S; AUG; AZT; CAX; CFT; CP; | aminoglycoside; fluoroquinolone; lactam; other | CP | fluoroquinolone |
| | CRM; GM; LVX; TO; T/S | | | |
| 32 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 33 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 34 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 35 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 36 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 37 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 38 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 39 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 40 | AM; A/S; AUG; AZT; CAX; CFT; CP; | aminoglycoside; fluoroquinolone; lactam; other | CP | fluoroquinolone |
| | CRM; GM; LVX; TO; T/S | | | |
| 41 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 42 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 43 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 44 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 45 | AM; A/S; AUG; AZT; CAX; CFT; CP; | aminoglycoside; fluoroquinolone; lactam; other | T/S | other |
| | CRM; GM; T/S | | | |
| 46 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 47 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 48 | AM; A/S; AUG; AZT; CAX; CFT; CP; | aminoglycoside; fluoroquinolone; lactam; other | LVX | fluoroquinolone |
| | CRM; GM; LVX; TO; T/S | | | |
| 49 | AM; A/S; AUG; T/S | lactam; other | T/S | other |
| 50 | AM; A/S; AUG; AZT; CAX; CFT; CP; | aminoglycoside; fluoroquinolone; lactam; other | LVX | fluoroquinolone |
| | CRM; GM; LVX; TO; T/S | | | |

TABLE 7c
Data for Escherichia, particularly E. coli (continued)

| No. | num_aminoglycoside | num_fluoroquinolone | num_lactam | num_other |
|---|---|---|---|---|
| 1 | 2 | 2 | 5 | 1 |
| 2 | 2 | 2 | 7 | 1 |
| 3 | 2 | 2 | 7 | 1 |
| 4 | 2 | 2 | 7 | 1 |
| 5 | 0 | 0 | 4 | 0 |
| 6 | 0 | 0 | 3 | 0 |
| 7 | 2 | 2 | 7 | 1 |
| 8 | 2 | 2 | 7 | 1 |
| 9 | 2 | 2 | 7 | 1 |
| 10 | 2 | 2 | 7 | 1 |
| 11 | 2 | 2 | 7 | 1 |
| 12 | 2 | 2 | 7 | 1 |
| 13 | 2 | 2 | 7 | 1 |
| 14 | 2 | 2 | 7 | 1 |
| 15 | 2 | 2 | 7 | 1 |
| 16 | 2 | 2 | 7 | 1 |
| 17 | 2 | 2 | 7 | 1 |
| 18 | 2 | 2 | 7 | 1 |
| 19 | 2 | 2 | 7 | 1 |
| 20 | 2 | 2 | 7 | 1 |
| 21 | 2 | 2 | 7 | 1 |
| 22 | 2 | 2 | 7 | 1 |
| 23 | 2 | 2 | 7 | 1 |
| 24 | 2 | 2 | 6 | 1 |
| 25 | 1 | 2 | 7 | 1 |
| 26 | 2 | 1 | 7 | 1 |
| 27 | 2 | 2 | 6 | 1 |
| 28 | 2 | 2 | 6 | 1 |
| 29 | 1 | 2 | 6 | 1 |
| 30 | 2 | 2 | 7 | 1 |
| 31 | 2 | 2 | 7 | 1 |
| 32 | 2 | 2 | 7 | 1 |
| 33 | 2 | 2 | 7 | 1 |
| 34 | 2 | 2 | 7 | 1 |
| 35 | 2 | 2 | 7 | 1 |
| 36 | 2 | 2 | 7 | 1 |
| 37 | 2 | 2 | 7 | 1 |
| 38 | 2 | 2 | 7 | 1 |
| 39 | 2 | 2 | 7 | 1 |
| 40 | 2 | 2 | 7 | 1 |
| 41 | 2 | 2 | 7 | 1 |
| 42 | 2 | 2 | 7 | 1 |
| 43 | 2 | 2 | 7 | 1 |
| 44 | 2 | 2 | 7 | 1 |
| 45 | 1 | 1 | 7 | 1 |
| 46 | 2 | 2 | 7 | 1 |
| 47 | 2 | 2 | 7 | 1 |
| 48 | 2 | 2 | 7 | 1 |
| 49 | 0 | 0 | 3 | 1 |
| 50 | 2 | 2 | 7 | 1 |

TABLE 7d
Data for Escherichia, particularly E. coli (continued)

| No. | AM_pv_adj | A/S_pv_adj | AUG_pv_adj | AZT_pv_adj |
|---|---|---|---|---|
| 1 | 3.50E−207 | 1.39E−175 | 2.23E−54 | 1.94E−02 |
| 2 | 3.12E−58 | 4.85E−48 | 1.56E−20 | 2.24E−14 |
| 3 | 2.85E−46 | 1.23E−39 | 2.49E−17 | 7.44E−15 |
| 4 | 2.62E−45 | 3.93E−39 | 1.12E−17 | 7.82E−14 |
| 5 | 1.24E−80 | 9.93E−72 | 2.04E−21 | 1.77E−02 |
| 6 | 2.42E−75 | 1.11E−66 | 8.58E−21 | 6.08E−02 |
| 7 | 1.01E−26 | 2.48E−22 | 5.21E−09 | 2.11E−04 |
| 8 | 4.88E−38 | 1.20E−35 | 3.67E−16 | 6.67E−10 |
| 9 | 2.35E−59 | 7.04E−49 | 2.73E−24 | 1.89E−06 |
| 10 | 2.35E−59 | 7.04E−49 | 1.31E−23 | 5.63E−06 |
| 11 | 1.84E−24 | 1.82E−23 | 1.33E−10 | 1.05E−06 |
| 12 | 2.51E−64 | 6.77E−56 | 3.88E−22 | 9.19E−04 |
| 13 | 2.79E−25 | 9.09E−23 | 1.32E−08 | 1.48E−03 |
| 14 | 2.94E−45 | 2.43E−34 | 1.15E−22 | 2.63E−14 |
| 15 | 1.58E−45 | 1.72E−34 | 1.75E−22 | 1.91E−14 |
| 16 | 3.26E−28 | 1.85E−21 | 2.00E−08 | 1.16E−05 |
| 17 | 1.44E−20 | 2.94E−18 | 5.11E−07 | 1.25E−04 |
| 18 | 6.63E−29 | 5.38E−22 | 1.16E−08 | 1.11E−05 |
| 19 | 6.41E−30 | 1.52E−22 | 9.58E−09 | 6.45E−06 |
| 20 | 4.62E−35 | 3.20E−30 | 5.19E−17 | 2.18E−07 |
| 21 | 1.82E−09 | 8.62E−11 | 2.50E−17 | 1.74E−39 |
| 22 | 3.88E−27 | 4.06E−27 | 4.62E−12 | 9.38E−06 |
| 23 | 1.09E−44 | 2.09E−39 | 1.05E−16 | 2.10E−06 |
| 24 | 2.75E−28 | 8.80E−22 | 3.79E−08 | 1.79E−02 |
| 25 | 6.10E−28 | 3.56E−20 | 2.25E−09 | 8.28E−04 |
| 26 | 5.97E−23 | 1.19E−15 | 1.39E−06 | 1.28E−03 |
| 27 | 4.17E−03 | 3.66E−02 | 4.90E−07 | 1.65E−14 |
| 28 | 4.17E−03 | 3.66E−02 | 4.90E−07 | 1.65E−14 |
| 29 | 1.37E−28 | 1.45E−21 | 8.41E−09 | 2.36E−02 |
| 30 | 5.88E−10 | 1.33E−07 | 1.85E−09 | 7.58E−11 |
| 31 | 1.14E−08 | 2.28E−05 | 5.66E−08 | 3.43E−16 |
| 32 | 1.96E−09 | 3.67E−07 | 4.28E−09 | 1.44E−10 |
| 33 | 2.88E−09 | 5.08E−07 | 4.50E−09 | 1.51E−10 |
| 34 | 1.07E−10 | 2.85E−08 | 1.31E−06 | 4.19E−06 |
| 35 | 4.32E−08 | 6.04E−05 | 1.49E−07 | 4.44E−16 |
| 36 | 8.33E−08 | 9.79E−05 | 2.97E−07 | 4.44E−16 |
| 37 | 8.33E−08 | 9.79E−05 | 2.97E−07 | 4.44E−16 |
| 38 | 3.07E−08 | 4.70E−05 | 1.02E−07 | 7.64E−16 |
| 39 | 4.14E−12 | 7.23E−10 | 1.20E−05 | 1.17E−03 |
| 40 | 1.57E−07 | 1.55E−04 | 4.17E−07 | 7.80E−16 |
| 41 | 2.15E−07 | 1.95E−04 | 5.61E−07 | 8.13E−16 |
| 42 | 1.55E−07 | 1.54E−04 | 3.95E−07 | 8.67E−16 |
| 43 | 1.55E−07 | 1.54E−04 | 3.95E−07 | 8.67E−16 |
| 44 | 1.55E−07 | 1.54E−04 | 3.95E−07 | 8.67E−16 |
| 45 | 3.13E−28 | 1.97E−20 | 2.60E−09 | 3.55E−04 |
| 46 | 1.16E−10 | 3.41E−08 | 3.47E−07 | 2.45E−06 |
| 47 | 2.72E−09 | 3.18E−07 | 2.44E−07 | 1.12E−10 |
| 48 | 2.66E−09 | 8.84E−08 | 1.21E−05 | 1.64E−08 |
| 49 | 4.47E−24 | 6.61E−18 | 2.77E−06 | 2.45E−01 |
| 50 | 2.16E−06 | 8.47E−04 | 1.36E−06 | 7.02E−16 |

TABLE 7e
Data for Escherichia, particularly E. coli (continued)

| No. | CAX_pv_adj | CFT_pv_adj | CP_pv_adj | CRM_pv_adj |
|---|---|---|---|---|
| 1 | 9.08E−03 | 5.54E−02 | 2.29E−17 | 2.63E−05 |
| 2 | 4.55E−21 | 9.31E−20 | 3.14E−43 | 1.15E−19 |
| 3 | 4.33E−19 | 1.87E−17 | 1.53E−32 | 5.07E−17 |
| 4 | 6.85E−18 | 2.28E−16 | 6.99E−31 | 7.62E−16 |
| 5 | 1.28E−02 | 7.14E−02 | 5.78E−01 | 3.21E−04 |
| 6 | 1.41E−01 | 3.53E−01 | 2.23E−01 | 3.75E−02 |
| 7 | 2.25E−06 | 3.51E−06 | 1.44E−38 | 1.55E−08 |
| 8 | 3.18E−15 | 1.35E−14 | 4.48E−11 | 1.70E−12 |
| 9 | 2.34E−05 | 5.15E−06 | 1.74E−15 | 7.72E−10 |
| 10 | 5.80E−05 | 9.14E−06 | 6.46E−15 | 2.18E−09 |
| 11 | 6.82E−10 | 3.64E−09 | 1.69E−49 | 1.37E−10 |
| 12 | 5.59E−03 | 1.88E−03 | 1.12E−09 | 2.39E−05 |
| 13 | 9.17E−05 | 2.37E−04 | 1.28E−36 | 6.73E−06 |
| 14 | 8.04E−16 | 5.19E−14 | 7.03E−61 | 9.05E−21 |
| 15 | 5.55E−16 | 2.31E−14 | 2.47E−60 | 6.03E−21 |
| 16 | 5.30E−07 | 2.10E−06 | 1.38E−39 | 8.69E−08 |
| 17 | 1.67E−06 | 3.95E−06 | 1.23E−38 | 5.25E−08 |
| 18 | 4.67E−07 | 1.87E−06 | 6.39E−39 | 5.20E−08 |
| 19 | 4.17E−07 | 1.68E−06 | 2.84E−39 | 4.28E−08 |
| 20 | 5.95E−11 | 4.86E−10 | 4.79E−07 | 1.46E−09 |
| 21 | 4.65E−46 | 9.26E−45 | 3.20E−19 | 1.06E−36 |
| 22 | 5.55E−08 | 2.77E−08 | 8.59E−09 | 1.64E−09 |
| 23 | 4.89E−09 | 9.33E−07 | 3.24E−25 | 8.32E−08 |
| 24 | 8.50E−03 | 9.80E−03 | 5.28E−05 | 4.13E−03 |
| 25 | 4.82E−05 | 7.11E−05 | 5.83E−03 | 5.89E−04 |
| 26 | 1.31E−04 | 1.77E−04 | 7.59E−03 | 2.84E−03 |
| 27 | 2.32E−15 | 2.75E−17 | 6.79E−43 | 2.56E−16 |
| 28 | 2.32E−15 | 2.75E−17 | 6.79E−43 | 2.56E−16 |
| 29 | 3.66E−03 | 4.47E−03 | 4.75E−03 | 2.48E−03 |
| 30 | 6.25E−11 | 6.54E−10 | 4.02E−42 | 2.80E−12 |
| 31 | 6.60E−17 | 2.67E−17 | 8.45E−42 | 2.23E−17 |
| 32 | 7.29E−11 | 7.76E−10 | 1.57E−41 | 5.31E−12 |
| 33 | 7.91E−11 | 8.46E−10 | 2.18E−41 | 1.70E−11 |
| 34 | 4.29E−06 | 1.52E−06 | 1.28E−40 | 1.80E−07 |
| 35 | 1.31E−16 | 5.36E−17 | 3.96E−41 | 4.26E−17 |
| 36 | 1.31E−16 | 5.36E−17 | 3.96E−41 | 4.26E−17 |
| 37 | 1.31E−16 | 5.36E−17 | 3.96E−41 | 4.26E−17 |
| 38 | 1.43E−16 | 5.77E−17 | 4.56E−41 | 1.17E−17 |
| 39 | 6.46E−05 | 1.74E−04 | 2.25E−40 | 1.80E−06 |
| 40 | 2.55E−16 | 1.06E−16 | 5.76E−41 | 7.39E−17 |
| 41 | 2.58E−16 | 1.06E−16 | 1.05E−40 | 7.84E−17 |
| 42 | 2.68E−16 | 1.10E−16 | 1.19E−40 | 2.23E−17 |
| 43 | 2.68E−16 | 1.10E−16 | 1.19E−40 | 2.23E−17 |
| 44 | 2.68E−16 | 1.10E−16 | 1.19E−40 | 2.23E−17 |
| 45 | 1.90E−05 | 2.89E−05 | 6.00E−03 | 1.99E−04 |
| 46 | 1.84E−06 | 5.25E−07 | 1.05E−39 | 6.76E−08 |
| 47 | 8.60E−10 | 4.43E−09 | 9.49E−40 | 8.85E−14 |
| 48 | 2.36E−11 | 1.27E−10 | 4.92E−38 | 2.22E−11 |
| 49 | 1.07E−01 | 1.14E−01 | 1.50E−02 | 6.49E−02 |
| 50 | 4.02E−16 | 1.67E−16 | 1.34E−38 | 7.42E−18 |

TABLE 7f
Data for Escherichia, particularly E. coli (continued)

| No. | GM_pv_adj | LVX_pv_adj | TO_pv_adj | T/S_pv_adj |
|---|---|---|---|---|
| 1 | 2.40E−14 | 2.96E−16 | 6.56E−10 | 5.59E−55 |
| 2 | 9.02E−31 | 5.84E−43 | 3.02E−26 | 1.62E−119 |
| 3 | 3.09E−28 | 1.25E−33 | 1.52E−27 | 6.25E−97 |
| 4 | 1.19E−27 | 5.56E−32 | 5.30E−27 | 8.78E−96 |
| 5 | 5.50E−01 | 4.74E−01 | 9.96E−02 | 3.77E−02 |
| 6 | 8.08E−01 | 2.88E−01 | 8.73E−01 | 1.21E−01 |
| 7 | 2.33E−13 | 1.57E−38 | 6.37E−15 | 7.68E−72 |
| 8 | 4.17E−11 | 5.22E−11 | 1.79E−08 | 1.58E−71 |
| 9 | 7.23E−11 | 6.17E−14 | 8.00E−05 | 1.01E−69 |
| 10 | 2.80E−10 | 1.39E−13 | 8.00E−05 | 1.23E−68 |
| 11 | 1.18E−18 | 2.49E−49 | 2.67E−22 | 6.77E−68 |
| 12 | 7.65E−07 | 1.10E−08 | 7.07E−03 | 1.95E−64 |
| 13 | 1.21E−16 | 1.92E−37 | 4.13E−18 | 1.87E−63 |
| 14 | 4.86E−26 | 7.43E−62 | 5.25E−32 | 2.58E−39 |
| 15 | 2.72E−26 | 3.08E−61 | 2.92E−32 | 4.30E−39 |
| 16 | 1.39E−16 | 1.68E−39 | 1.59E−17 | 1.74E−60 |
| 17 | 5.78E−18 | 2.01E−39 | 1.91E−19 | 2.44E−60 |
| 18 | 1.02E−16 | 7.87E−39 | 1.17E−17 | 6.74E−60 |
| 19 | 7.38E−17 | 3.56E−39 | 8.56E−18 | 2.63E−59 |
| 20 | 6.73E−07 | 9.82E−07 | 5.94E−07 | 3.47E−47 |
| 21 | 5.34E−12 | 1.45E−19 | 7.00E−09 | 2.74E−06 |
| 22 | 3.89E−07 | 1.14E−08 | 2.09E−05 | 7.71E−46 |
| 23 | 6.04E−13 | 2.10E−24 | 3.47E−12 | 1.02E−37 |
| 24 | 8.52E−06 | 1.56E−04 | 7.54E−03 | 4.90E−44 |
| 25 | 9.39E−05 | 9.80E−03 | 1.28E−02 | 6.98E−44 |
| 26 | 2.16E−04 | 1.65E−02 | 9.49E−03 | 9.68E−44 |
| 27 | 1.81E−12 | 3.58E−43 | 2.05E−13 | 2.13E−06 |
| 28 | 1.81E−12 | 3.58E−43 | 2.05E−13 | 2.13E−06 |
| 29 | 1.08E−04 | 8.15E−03 | 5.44E−02 | 5.54E−43 |
| 30 | 1.81E−12 | 2.71E−42 | 4.67E−11 | 6.64E−17 |
| 31 | 3.65E−19 | 9.37E−42 | 2.80E−17 | 3.20E−18 |
| 32 | 3.61E−12 | 1.03E−41 | 9.37E−11 | 2.18E−16 |
| 33 | 3.86E−12 | 1.47E−41 | 9.73E−11 | 2.45E−16 |
| 34 | 1.03E−13 | 2.44E−41 | 2.22E−16 | 4.82E−04 |
| 35 | 4.79E−19 | 2.61E−41 | 5.83E−17 | 9.95E−18 |
| 36 | 4.79E−19 | 2.61E−41 | 5.83E−17 | 3.03E−17 |
| 37 | 4.79E−19 | 2.61E−41 | 5.83E−17 | 3.03E−17 |
| 38 | 8.47E−19 | 3.21E−41 | 6.04E−17 | 3.36E−18 |
| 39 | 3.56E−12 | 3.69E−41 | 8.59E−15 | 1.04E−04 |
| 40 | 8.74E−19 | 6.20E−41 | 6.42E−17 | 5.21E−17 |
| 41 | 9.35E−19 | 6.76E−41 | 6.96E−17 | 8.31E−17 |
| 42 | 1.03E−18 | 8.13E−41 | 1.27E−16 | 2.95E−17 |
| 43 | 1.03E−18 | 8.13E−41 | 1.27E−16 | 2.95E−17 |
| 44 | 1.03E−18 | 8.13E−41 | 1.27E−16 | 2.95E−17 |
| 45 | 9.79E−05 | 1.29E−02 | 1.33E−02 | 1.07E−40 |
| 46 | 1.98E−13 | 1.85E−40 | 4.73E−16 | 4.17E−04 |
| 47 | 2.68E−16 | 1.90E−40 | 1.10E−17 | 2.80E−08 |
| 48 | 1.27E−13 | 3.36E−40 | 7.14E−14 | 3.41E−07 |
| 49 | 1.15E−02 | 2.48E−02 | 4.44E−01 | 3.77E−40 |
| 50 | 1.50E−18 | 8.68E−39 | 9.76E−17 | 2.56E−15 |

TABLE 7g
Data for Escherichia, particularly E. coli (continued)

| No. | Locus.Tag | Gene.Symbol | GenBank.Accession | Chromosome |
|---|---|---|---|---|
| 1 | SeHA_C1580 | blaT | YP_002045448 | |
| 2 | EcolB7_01001258 | | ZP_00717086 | |
| 3 | HMPREF9345_05066 | | ZP_07100147 | |
| 4 | KPN_pKPN5p08201 | | YP_001338811 | plasmid pKPN5 |
| 5 | HMPREF9547_03104 | | ZP_07169558 | |
| 6 | SeSA_B0079 | | YP_002112958 | plasmid |
| | | | | pCVM19633_110 |
| 7 | HMPREF9345_05232 | | ZP_07100311 | |
| 8 | EcolB_01004576 | | ZP_00708527 | |
| 9 | pEFER_0049 | | YP_002394596 | plasmid pEFER |
| 10 | ETAE_p040 | | YP_003297634 | plasmid pEIB202 |
| 11 | ECGG_04429 | | ZP_06648541 | |
| 12 | ETAE_p041 | folP | YP_003297635 | plasmid pEIB202 |
| 13 | ECGG_04434 | | ZP_06648545 | |
| 14 | ROD_p1051 | pemI | YP_003368450 | plasmid pCROD1 |
| 15 | ROD_p1061 | pemK | YP_003368451 | plasmid pCROD1 |
| 16 | HMPREF9348_05540 | | ZP_07692671 | |
| 17 | ECUMN_4816 | aadA | YP_002415408 | |
| 18 | ECUMN_4827 | mphA | YP_002415417 | |
| 19 | HMPREF9345_05223 | | ZP_07100302 | |
| 20 | EcSMS35_A0150 | | YP_001740030 | plasmid pSMS35_130 |
| 21 | HMPREF9548_03778 | | ZP_07141582 | |
| 22 | HMPREF9348_05561 | | ZP_07692692 | |
| 23 | HMPREF9548_03784 | | ZP_07141588 | |
| 24 | KPN_pKPN4p07064 | | YP_001338673 | plasmid pKPN4 |
| 25 | HMPREF9345_03525 | | ZP_07098656 | |
| 26 | KPN_pKPN4p07068 | merC | YP_001338677 | plasmid pKPN4 |
| 27 | ESCG_01010 | | ZP_04871321 | |
| 28 | HMPREF9540_00466 | | ZP_07133313 | |
| 29 | STMDT12_C39340 | | BAJ38877 | |
| 30 | KPK_A0022 | sopB | YP_002235674 | plasmid pKP187 |
| 31 | EcoliO157_010100014809 | | ZP_02780941 | |
| 32 | EcSMS35_A0072 | sopB | YP_001739960 | plasmid pSMS35_130 |
| 33 | HMPREF9538_00090 | | ZP_08302456 | |
| 34 | ECNA114_2444 | | AEG37278 | |
| 35 | EschericcoliO157_010100006568 | | ZP_02749755 | |
| 36 | CSAG_00128 | | ZP_04560798 | |
| 37 | ECH74115_0422 | mhpA | YP_002268988 | |
| 38 | CIT292_01335 | | ZP_03835561 | |
| 39 | ECSF_1165 | | BAI54705 | |
| 40 | EscherichiacoliO157EcO_010100009454 | | ZP_05948572 | |
| 41 | SSON_0329 | mhpD | YP_309341 | |
| 42 | CIT292_01336 | | ZP_03835562 | |
| 43 | HMPREF9540_00984 | | ZP_07133822 | |
| 44 | Missing | mhpE | AP_001004 | |
| | locus_tag:AC_000091.cds340.373092..374105 | | | |
| 45 | HMPREF9551_04611 | | ZP_07191964 | |
| 46 | SeHA_C2638 | | YP_002046450 | |
| 47 | SFxv_0758 | | ADA73054 | |
| 48 | HMPREF9530_01431 | | ZP_07151340 | |
| 49 | KPN_pKPN4p07069 | merA | YP_001338678 | plasmid pKPN4 |
| 50 | CP0077 | | NP_858210 | plasmid pCP301 |

TABLE 7h
Data for Escherichia, particularly E. coli (continued)

| No. | Start.Coord | End.Coord | Strand | Scaffold.ID |
|---|---|---|---|---|
| 1 | 1525960 | 1526820 | + | 642555218 |
| 2 | 77515 | 77910 | + | 638358454 |
| 3 | 4715 | 5554 | − | 648294624 |
| 4 | 34908 | 35255 | − | 640753021 |
| 5 | 3248 | 3841 | + | 648291919 |
| 6 | 68933 | 70240 | + | 642555219 |
| 7 | 5252 | 5572 | − | 648294635 |
| 8 | 3555 | 4568 | + | 638359041 |
| 9 | 48658 | 49503 | + | 643692014 |
| 10 | 31237 | 32040 | − | 646311959 |
| 11 | 19278 | 19826 | + | 647536609 |
| 12 | 32101 | 32916 | − | 646311959 |
| 13 | 22618 | 23823 | + | 647536609 |
| 14 | 3339 | 3596 | + | 646311988 |
| 15 | 3529 | 3930 | + | 646311988 |
| 16 | 461 | 1045 | − | 649994953 |
| 17 | 4994882 | 4995670 | + | 644736335 |
| 18 | 5003053 | 5003958 | − | 644736335 |
| 19 | 1167 | 2405 | + | 648294635 |
| 20 | 118542 | 119474 | + | 641522668 |
| 21 | 4493 | 5755 | + | 648288593 |
| 22 | 209 | 661 | + | 649994959 |
| 23 | 8819 | 9535 | + | 648288593 |
| 24 | 23310 | 24614 | − | 640753020 |
| 25 | 1071 | 1307 | − | 648294596 |
| 26 | 25970 | 27664 | − | 640753020 |
| 27 | 241682 | 242032 | + | 646206991 |
| 28 | 192 | 443 | + | 648287910 |
| 29 | 4084530 | 4085237 | − | 651053007 |
| 30 | 12106 | 13071 | − | 643348532 |
| 31 | 123136 | 123969 | − | 641781714 |
| 32 | 53664 | 54635 | − | 641522668 |
| 33 | 5657 | 6832 | + | 651332930 |
| 34 | 2476906 | 2477910 | − | 651053098 |
| 35 | 369676 | 370620 | + | 641780393 |
| 36 | 146099 | 147763 | + | 646206732 |
| 37 | 430810 | 432474 | + | 643348548 |
| 38 | 25505 | 26449 | + | 643891503 |
| 39 | 1248345 | 1249289 | + | 646862305 |
| 40 | 20015 | 20965 | + | 645964942 |
| 41 | 356486 | 357301 | + | 640427102 |
| 42 | 26463 | 27344 | + | 643891503 |
| 43 | 7204 | 8217 | + | 648287942 |
| 44 | 373092 | 374105 | + | 637000000 |
| 45 | 97 | 531 | − | 648293053 |
| 46 | 2550781 | 2552010 | + | 642555218 |
| 47 | 734541 | 734921 | + | 646862363 |
| 48 | 8 | 643 | − | 648290358 |
| 49 | 27716 | 28177 | − | 640753020 |
| 50 | 61397 | 61579 | + | 637000224 |

TABLE 8a
Data for Escherichia, particularly E. coli

| | Scaffold.External. | | |
|---|---|---|---|
| No. | Accession | Scaffold.Name | best_pv |
| 1 | NC_011083 | Salmonella enterica subsp. enterica serovar Heidelberg | 3.5041948925527e−207 |
| | | str. SL476: NC_011083 | |
| 2 | NZ_AAJT01000012 | Escherichia coli B7A, unfinished sequence: NZ_AAJT01000012 | 1.62252621621877e−119 |
| 3 | NZ_ADWV01000045 | Escherichia coli MS 107-1 E_coliMS107-1-1.0.1_Cont44.1: | 6.24750842070913e−97 |
| | | NZ_ADWV01000045 | |
| 4 | NC_009651 | Klebsiella pneumoniae subsp. pneumoniae MGH 78578 plasmid | 8.77735582031733e−96 |
| | | pKPN5: NC_009651 | |
| 5 | NZ_ADWV01000056 | Escherichia coli MS 107-1 E_coliMS107-1-1.0.1_Cont55.1: | 7.68091736117399e−72 |
| | | NZ_ADWV01000056 | |
| 6 | NZ_AAJX01000089 | Escherichia coli B171, unfinished sequence: | 1.57772112100981e−71 |
| | | NZ_AAJX01000089 | |
| 7 | NC_011743 | Escherichia fergusonii ATCC 35469 plasmid pEFER: NC_011743 | 1.0118246517332e−69 |
| 8 | NC_013509 | Edwardsiella tarda EIB202 plasmid pEIB202: NC_013509 | 1.23255842888535e−68 |
| 9 | NZ_GG749218 | Escherichia coli FVEC1412 genomic scaffold supercont1.13: | 6.77313303746994e−68 |
| | | NZ_GG749218 | |
| 10 | NC_013509 | Edwardsiella tarda EIB202 plasmid pEIB202: NC_013509 | 1.94619564613577e−64 |
| 11 | NZ_GG749218 | Escherichia coli FVEC1412 genomic scaffold supercont1.13: | 1.86737834552034e−63 |
| | | NZ_GG749218 | |
| 12 | NC_013717 | Citrobacter rodentium ICC168 plasmid pCROD1: NC_013717 | 7.42780365363135e−62 |
| 13 | NC_013717 | Citrobacter rodentium ICC168 plasmid pCROD1: NC_013717 | 3.07751376659308e−61 |
| 14 | NZ_ADWS01000089 | Escherichia coli MS 145-7 E_coliMS145-7-1.0.1_Cont88.1: | 1.7351795845908e−60 |
| | | NZ_ADWS01000089 | |
| 15 | NC_011751 | Escherichia coli UMN026: NC_011751 | 2.44317906034667e−60 |
| 16 | NC_011751 | Escherichia coli UMN026: NC_011751 | 6.73588199342071e−60 |
| 17 | NZ_ADWV01000056 | Escherichia coli MS 107-1 E_coliMS107-1-1.0.1_Cont55.1: | 2.62943019143703e−59 |
| | | NZ_ADWV01000056 | |
| 18 | NC_010488 | Escherichia coli SECEC SMS-3-5 plasmid pSMS35_130: | 3.46702381924878e−47 |
| | | NC_010488 | |
| 19 | NZ_ADTM01000311 | Escherichia coli MS 182-1 E_coli182-1-1.0_Cont460.3: | 4.65308035729244e−46 |
| | | NZ_ADTM01000311 | |
| 20 | NZ_ADWS01000095 | Escherichia coli MS 145-7 E_coliMS145-7-1.0.1_Cont94.1: | 7.71451120848483e−46 |
| | | NZ_ADWS01000095 | |
| 21 | NZ_ADTM01000311 | Escherichia coli MS 182-1 E_coli182-1-1.0_Cont460.3: | 1.08551755499227e−44 |
| | | NZ_ADTM01000311 | |
| 22 | NC_009650 | Klebsiella pneumoniae subsp. pneumoniae MGH 78578 plasmid | 4.89761264283562e−44 |
| | | pKPN4: NC_009650 | |
| 23 | NZ_ADWV01000017 | Escherichia coli MS 107-1 E_coliMS107-1-1.0.1_Cont16.1: | 6.97693050178087e−44 |
| | | NZ_ADWV01000017 | |
| 24 | NC_009650 | Klebsiella pneumoniae subsp. pneumoniae MGH 78578 plasmid | 9.68294892967391e−44 |
| | | pKPN4: NC_009650 | |
| 25 | NZ_GG665811 | Escherichia sp. 1_1_43 genomic scaffold supercont1.1: | 3.5843867496487e−43 |
| | | NZ_GG665811 | |
| 26 | NZ_ADTL01000051 | Escherichia coli MS 115-1 E_coli115-1-1.0_Cont158.2: | 3.5843867496487e−43 |
| | | NZ_ADTL01000051 | |
| 27 | AP011957 | Salmonella enterica subsp. enterica serovar Typhimurium | 5.53805522527039e−43 |
| | | str. T000240 DNA: AP011957 | |
| 28 | NC_011282 | Klebsiella pneumoniae 342 plasmid pKP187: NC_011282 | 2.71226651272988e−42 |
| 29 | NZ_ABHR01000011 | Escherichia coli O157:H7 str. EC4401, unfinished sequence: | 8.45082785112866e−42 |
| | | NZ_ABHR01000011 | |
| 30 | NC_010488 | Escherichia coli SECEC SMS-3-5 plasmid pSMS35_130: | 1.02749291231853e−41 |
| | | NC_010488 | |
| 31 | NZ_AFBO01000013 | Klebsiella sp. MS 92-3 K_spMS92-3-l.0_Cont24.1: | 1.46909169086738e−41 |
| | | NZ_AFBO01000013 | |
| 32 | CP002797 | Escherichia coli NA114: CP002797 | 2.43588122712712e−41 |
| 33 | NZ_ABHK01000002 | Escherichia coli O157:H7 str. EC4206, unfinished sequence: | 2.60702932643171e−41 |
| | | NZ_ABHK01000002 | |
| 34 | NZ_GG657366 | Citrobacter sp. 30_2 genomic scaffold supercont1.1: | 2.60702932643171e−41 |
| | | NZ_GG657366 | |
| 35 | NC_011353 | Escherichia coli O157:H7 str. EC4115: NC_011353 | 2.60702932643171e−41 |
| 36 | NZ_ABWL01000012 | Citrobacter youngae ATCC 29220 C_sp-1.0_Cont2.3: | 3.21133372679738e−41 |
| | | NZ_ABWL01000012 | |
| 37 | AP009378 | Escherichia coli SE15 DNA: AP009378 | 3.68640118720819e−41 |
| 38 | NZ_ACXN01000074 | Escherichia coli O157:H7 str. FRIK966: NZ_ACXN01000074 | 5.75695041652081e−41 |
| 39 | NC_007384 | Shigella sonnei Ss046: NC_007384 | 6.75951367822551e−41 |
| 40 | NZ_ABWL01000012 | Citrobacter youngae ATCC 29220 C_sp-1.0_Cont2.3: | 8.12681193093067e−41 |
| | | NZ_ABWL01000012 | |
| 41 | NZ_ADTL01000083 | Escherichia coli MS 115-1 E_coli115-1-1.0_Cont264.1: | 8.12681193093067e−41 |
| | | NZ_ADTL01000083 | |
| 42 | AC_000091 | Escherichia coli W3110 DNA: AC_000091 | 8.12681193093067e−41 |
| 43 | NZ_ADUD01000537 | Escherichia coli MS 196-1 E_coli196-1-1.0_Cont1482.1: | 1.06521634399457e−40 |
| | | NZ_ADUD01000537 | |
| 44 | NC_011083 | Salmonella enterica subsp. enterica serovar Heidelberg | 1.84998224240006e−40 |
| | | str. SL476: NC_011083 | |
| 45 | CP001383 | Shigella flexneri 2002017: CP001383 | 1.89942261631873e−40 |
| 46 | NZ_ADTR01000134 | Escherichia coli MS 21-1 E_coli21-1-1.0_Cont296.1: | 3.35939290103889e−40 |
| | | NZ_ADTR01000134 | |
| 47 | NC_009650 | Klebsiella pneumoniae subsp. pneumoniae MGH 78578 plasmid | 3.76989142366575e−40 |
| | | pKPN4: NC_009650 | |
| 48 | NC_004851 | Shigella flexneri 2a str. 301 plasmid pCP301: NC_004851 | 8.68449218790606e−39 |
| 49 | NC_007607 | Shigella dysenteriae Sd197 plasmid pSD1_197: NC_007607 | 2.27746826869945e−38 |
| 50 | NZ_ADTL01000280 | Escherichia coli MS 115-1 E_coli115-1-1.0_Cont677.5: | 4.92053652141975e−38 |
| | | NZ_ADTL01000280 | |

TABLE 8b
Data for Escherichia, particularly E. coli (continued)

| No. | sign_phenos | sign_phenos_class | best_pheno | best_pheno_class |
|---|---|---|---|---|
| 1 | AM; A/S; AUG; CAX; CP; CRM; GM; LVX; TO; T/S | aminoglycoside; fluoroquinolone; lactam; other | AM | lactam |
| 2 | AM; A/S; AUG; AZ T; CAX; CFT; CP; CRM; GM; LVX; | aminoglycoside; fluoroquinolone; lactam; other | T/S | other |
| | TO; T/S | | | |
| 3 | AM; A/S; AUG; AZT; CAX; CFT; CP; CRM; GM; LVX; | aminoglycoside; fluoroquinolone; lactam; other | T/S | other |
| | TO; T/S | | | |
| 4 | AM; A/S; AUG; AZT; CAX; CFT; CP; CRM; GM; LVX; | aminoglycoside; fluoroquinolone; lactam; other | T/S | other |
| | TO; T/S | | | |
| 5 | AM; A/S; AUG; AZT; CAX; CFT; CP; CRM; GM; LVX; | aminoglycoside; fluoroquinolone; lactam; other | T/S | other |
| | TO; T/S | | | |
| 6 | AM; A/S; AUG; AZT; CAX; CFT; CP; CRM; GM; LVX; | aminoglycoside; fluoroquinolone; lactam; other | T/S | other |
| | TO; T/S | | | |
| 7 | AM; A/S; AUG; AZT; CAX; CFT; CP; CRM; GM; LVX; | aminoglycoside; fluoroquinolone; lactam; other | T/S | other |
| | TO; T/S | | | |
| 8 | AM; A/S; AUG; AZT; CAX; CFT; CP; CRM; GM; LVX; | aminoglycoside; fluoroquinolone; lactam; other | T/S | other |
| | TO; T/S | | | |
| 9 | AM; A/S; AUG; AZT; CAX; CFT; CP; CRM; GM; LVX; | aminoglycoside; fluoroquinolone; lactam; other | T/S | other |
| | TO; T/S | | | |
| 10 | AM; A/S; AUG; AZT; CAX; CFT; CP; CRM; GM; LVX; | aminoglycoside; fluoroquinolone; lactam; other | T/S | other |
| | TO; T/S | | | |
| 11 | AM; A/S; AUG; AZT; CAX; CFT; CP; CRM; GM; LVX; | aminoglycoside; fluoroquinolone; lactam; other | T/S | other |
| | TO; T/S | | | |
| 12 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 13 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 14 | AM; A/S; AUG; AZT; CAX; CFT; CP; CRM; GM; LVX; | aminoglycoside; fluoroquinolone; lactam; other | T/S | other |
| | TO; T/S | | | |
| 15 | AM; A/S; AUG; AZT; CAX; CFT; CP; CRM; GM; LVX; | aminoglycoside; fluoroquinolone; lactam; other | T/S | other |
| | TO; T/S | | | |
| 16 | AM; A/S; AUG; AZT; CAX; CFT; CP; CRM; GM; LVX; | aminoglycoside; fluoroquinolone; lactam; other | T/S | other |
| | TO; T/S | | | |
| 17 | AM; A/S; AUG; AZT; CAX; CFT; CP; CRM; GM; LVX; | aminoglycoside; fluoroquinolone; lactam; other | T/S | other |
| | TO; T/S | | | |
| 18 | AM; A/S; AUG; AZT; CAX; CFT; CP; CRM; GM; LVX; | aminoglycoside; fluoroquinolone; lactam; other | T/S | other |
| | TO; T/S | | | |
| 19 | AM; A/S; AUG; AZT; CAX; CFT; CP; CRM; GM; LVX; | aminoglycoside; fluoroquinolone; lactam; other | CAX | lactam |
| | TO; T/S | | | |
| 20 | AM; A/S; AUG; AZT; CAX; CFT; CP; CRM; GM; LVX; | aminoglycoside; fluoroquinolone; lactam; other | T/S | other |
| | TO; T/S | | | |
| 21 | AM; A/S; AUG; AZT; CAX; CFT; CP; CRM; GM; LVX; | aminoglycoside; fluoroquinolone; lactam; other | AM | lactam |
| | TO; T/S | | | |
| 22 | AM; A/S; AUG; CAX; CFT; CP; CRM; GM; LVX; TO; | aminoglycoside; fluoroquinolone; lactam; other | T/S | other |
| | T/S | | | |
| 23 | AM; A/S; AUG; AZT; CAX; CFT; CP; CRM; GM; LVX; | aminoglycoside; fluoroquinolone; lactam; other | T/S | other |
| | T/S | | | |
| 24 | AM; A/S; AUG; AZT; CAX; CFT; CP; CRM; GM; TO; | aminoglycoside; fluoroquinolone; lactam; other | T/S | other |
| | T/S | | | |
| 25 | AM; AUG; AZT; CAX; CFT; CP; CRM; GM; LVX; TO; | aminoglycoside; fluoroquinolone; lactam; other | LVX | fluoroquinolone |
| | T/S | | | |
| 26 | AM; AUG; AZT; CAX; CFT; CP; CRM; GM; LVX; TO; | aminoglycoside; fluoroquinolone; lactam; other | LVX | fluoroquinolone |
| | T/S | | | |
| 27 | AM; A/S; AUG; CAX; CFT; CP; CRM; GM; LVX; T/S | aminoglycoside; fluoroquinolone; lactam; other | T/S | other |
| 28 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 29 | AM; A/S; AUG; AZT; CAX; CFT; CP; CRM; GM; LVX; | aminoglycoside; fluoroquinolone; lactam; other | CP | fluoroquinolone |
| | TO; T/S | | | |
| 30 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 31 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 32 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 33 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 34 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 35 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 36 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 37 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 38 | AM; A/S; AUG; AZT; CAX; CFT; CP; CRM; GM; LVX; | aminoglycoside; fluoroquinolone; lactam; other | CP | fluoroquinolone |
| | TO; T/S | | | |
| 39 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 40 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 41 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 42 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 43 | AM; A/S; AUG; AZT; CAX; CFT; CP; CRM; GM; T/S | aminoglycoside; fluoroquinolone; lactam; other | T/S | other |
| 44 | AM; A/S; AUG; AZT; CAX; CFT; CP; C RM; GM; LVX; | aminoglycoside; fluoroquinolone; lactam; other | LVX | fluoroquinolone |
| | TO; T/S | | | |
| 45 | AM; A/S; AUG; AZT; CAX; CFT; CP; C RM; GM; LVX; | aminoglycoside; fluoroquinolone; lactam; other | LVX | fluoroquinolone |
| | TO; T/S | | | |
| 46 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 47 | AM; A/S; AUG; T/S | lactam; other | T/S | other |
| 48 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 49 | AM; A/S; AUG; AZT; CAX; CFT; CP; CRM; GM; LVX; | aminoglycoside; fluoroquinolone; lactam; other | LVX | fluoroquinolone |
| | TO; T/S | | | |
| 50 | AM; A/S; AUG; AZT; CAX; CFT; CP; CRM; GM; LVX; | aminoglycoside; fluoroquinolone; lactam; other | LVX | fluoroquinolone |
| | TO; T/S | | | |

TABLE 8c
Data for Escherichia, particularly E. coli (continued)

| No. | num_aminoglycoside | num_fluoroquinolone | num_lactam | num_other |
|---|---|---|---|---|
| 1 | 2 | 2 | 5 | 1 |
| 2 | 2 | 2 | 7 | 1 |
| 3 | 2 | 2 | 7 | 1 |
| 4 | 2 | 2 | 7 | 1 |
| 5 | 2 | 2 | 7 | 1 |
| 6 | 2 | 2 | 7 | 1 |
| 7 | 2 | 2 | 7 | 1 |
| 8 | 2 | 2 | 7 | 1 |
| 9 | 2 | 2 | 7 | 1 |
| 10 | 2 | 2 | 7 | 1 |
| 11 | 2 | 2 | 7 | 1 |
| 12 | 2 | 2 | 7 | 1 |
| 13 | 2 | 2 | 7 | 1 |
| 14 | 2 | 2 | 7 | 1 |
| 15 | 2 | 2 | 7 | 1 |
| 16 | 2 | 2 | 7 | 1 |
| 17 | 2 | 2 | 7 | 1 |
| 18 | 2 | 2 | 7 | 1 |
| 19 | 2 | 2 | 7 | 1 |
| 20 | 2 | 2 | 7 | 1 |
| 21 | 2 | 2 | 7 | 1 |
| 22 | 2 | 2 | 6 | 1 |
| 23 | 1 | 2 | 7 | 1 |
| 24 | 2 | 1 | 7 | 1 |
| 25 | 2 | 2 | 6 | 1 |
| 26 | 2 | 2 | 6 | 1 |
| 27 | 1 | 2 | 6 | 1 |
| 28 | 2 | 2 | 7 | 1 |
| 29 | 2 | 2 | 7 | 1 |
| 30 | 2 | 2 | 7 | 1 |
| 31 | 2 | 2 | 7 | 1 |
| 32 | 2 | 2 | 7 | 1 |
| 33 | 2 | 2 | 7 | 1 |
| 34 | 2 | 2 | 7 | 1 |
| 35 | 2 | 2 | 7 | 1 |
| 36 | 2 | 2 | 7 | 1 |
| 37 | 2 | 2 | 7 | 1 |
| 38 | 2 | 2 | 7 | 1 |
| 39 | 2 | 2 | 7 | 1 |
| 40 | 2 | 2 | 7 | 1 |
| 41 | 2 | 2 | 7 | 1 |
| 42 | 2 | 2 | 7 | 1 |
| 43 | 1 | 1 | 7 | 1 |
| 44 | 2 | 2 | 7 | 1 |
| 45 | 2 | 2 | 7 | 1 |
| 46 | 2 | 2 | 7 | 1 |
| 47 | 0 | 0 | 3 | 1 |
| 48 | 2 | 2 | 7 | 1 |
| 49 | 2 | 2 | 7 | 1 |
| 50 | 2 | 2 | 7 | 1 |

TABLE 8d
Data for Escherichia, particularly E. coli (continued)

| No. | AM_pv_adj | A/S_pv_adj | AUG_pv_adj | AZT_pv_adj |
|---|---|---|---|---|
| 1 | 3.50E−207 | 1.39E−175 | 2.23E−54 | 1.94E−02 |
| 2 | 3.12E−58 | 4.85E−48 | 1.56E−20 | 2.24E−14 |
| 3 | 2.85E−46 | 1.23E−39 | 2.49E−17 | 7.44E−15 |
| 4 | 2.62E−45 | 3.93E−39 | 1.12E−17 | 7.82E−14 |
| 5 | 1.01E−26 | 2.48E−22 | 5.21E−09 | 2.11E−04 |
| 6 | 4.88E−38 | 1.20E−35 | 3.67E−16 | 6.67E−10 |
| 7 | 2.35E−59 | 7.04E−49 | 2.73E−24 | 1.89E−06 |
| 8 | 2.35E−59 | 7.04E−49 | 1.31E−23 | 5.63E−06 |
| 9 | 1.84E−24 | 1.82E−23 | 1.33E−10 | 1.05E−06 |
| 10 | 2.51E−64 | 6.77E−56 | 3.88E−22 | 9.19E−04 |
| 11 | 2.79E−25 | 9.09E−23 | 1.32E−08 | 1.48E−03 |
| 12 | 2.94E−45 | 2.43E−34 | 1.15E−22 | 2.63E−14 |
| 13 | 1.58E−45 | 1.72E−34 | 1.75E−22 | 1.91E−14 |
| 14 | 3.26E−28 | 1.85E−21 | 2.00E−08 | 1.16E−05 |
| 15 | 1.44E−20 | 2.94E−18 | 5.11E−07 | 1.25E−04 |
| 16 | 6.63E−29 | 5.38E−22 | 1.16E−08 | 1.11E−05 |
| 17 | 6.41E−30 | 1.52E−22 | 9.58E−09 | 6.45E−06 |
| 18 | 4.62E−35 | 3.20E−30 | 5.19E−17 | 2.18E−07 |
| 19 | 1.82E−09 | 8.62E−11 | 2.50E−17 | 1.74E−39 |
| 20 | 3.88E−27 | 4.06E−27 | 4.62E−12 | 9.38E−06 |
| 21 | 1.09E−44 | 2.09E−39 | 1.05E−16 | 2.10E−06 |
| 22 | 2.75E−28 | 8.80E−22 | 3.79E−08 | 1.79E−02 |
| 23 | 6.10E−28 | 3.56E−20 | 2.25E−09 | 8.28E−04 |
| 24 | 5.97E−23 | 1.19E−15 | 1.39E−06 | 1.28E−03 |
| 25 | 4.17E−03 | 3.66E−02 | 4.90E−07 | 1.65E−14 |
| 26 | 4.17E−03 | 3.66E−02 | 4.90E−07 | 1.65E−14 |
| 27 | 1.37E−28 | 1.45E−21 | 8.41E−09 | 2.36E−02 |
| 28 | 5.88E−10 | 1.33E−07 | 1.85E−09 | 7.58E−11 |
| 29 | 1.14E−08 | 2.28E−05 | 5.66E−08 | 3.43E−16 |
| 30 | 1.96E−09 | 3.67E−07 | 4.28E−09 | 1.44E−10 |
| 31 | 2.88E−09 | 5.08E−07 | 4.50E−09 | 1.51E−10 |
| 32 | 1.07E−10 | 2.85E−08 | 1.31E−06 | 4.19E−06 |
| 33 | 4.32E−08 | 6.04E−05 | 1.49E−07 | 4.44E−16 |
| 34 | 8.33E−08 | 9.79E−05 | 2.97E−07 | 4.44E−16 |
| 35 | 8.33E−08 | 9.79E−05 | 2.97E−07 | 4.44E−16 |
| 36 | 3.07E−08 | 4.70E−05 | 1.02E−07 | 7.64E−16 |
| 37 | 4.14E−12 | 7.23E−10 | 1.20E−05 | 1.17E−03 |
| 38 | 1.57E−07 | 1.55E−04 | 4.17E−07 | 7.80E−16 |
| 39 | 2.15E−07 | 1.95E−04 | 5.61E−07 | 8.13E−16 |
| 40 | 1.55E−07 | 1.54E−04 | 3.95E−07 | 8.67E−16 |
| 41 | 1.55E−07 | 1.54E−04 | 3.95E−07 | 8.67E−16 |
| 42 | 1.55E−07 | 1.54E−04 | 3.95E−07 | 8.67E−16 |
| 43 | 3.13E−28 | 1.97E−20 | 2.60E−09 | 3.55E−04 |
| 44 | 1.16E−10 | 3.41E−08 | 3.47E−07 | 2.45E−06 |
| 45 | 2.72E−09 | 3.18E−07 | 2.44E−07 | 1.12E−10 |
| 46 | 2.66E−09 | 8.84E−08 | 1.21E−05 | 1.64E−08 |
| 47 | 4.47E−24 | 6.61E−18 | 2.77E−06 | 2.45E−01 |
| 48 | 2.16E−06 | 8.47E−04 | 1.36E−06 | 7.02E−16 |
| 49 | 4.99E−06 | 1.53E−03 | 2.46E−06 | 1.26E−15 |
| 50 | 1.11E−05 | 1.86E−03 | 1.74E−06 | 1.63E−14 |

TABLE 8e
Data for Escherichia, particularly E. coli (continued)

| No. | CAX_pv_adj | CFT_pv_adj | CP_pv_adj | CRM_pv_adj |
|---|---|---|---|---|
| 1 | 9.08E−03 | 5.54E−02 | 2.29E−17 | 2.63E−05 |
| 2 | 4.55E−21 | 9.31E−20 | 3.14E−43 | 1.15E−19 |
| 3 | 4.33E−19 | 1.87E−17 | 1.53E−32 | 5.07E−17 |
| 4 | 6.85E−18 | 2.28E−16 | 6.99E−31 | 7.62E−16 |
| 5 | 2.25E−06 | 3.51E−06 | 1.44E−38 | 1.55E−08 |
| 6 | 3.18E−15 | 1.35E−14 | 4.48E−11 | 1.70E−12 |
| 7 | 2.34E−05 | 5.15E−06 | 1.74E−15 | 7.72E−10 |
| 8 | 5.80E−05 | 9.14E−06 | 6.46E−15 | 2.18E−09 |
| 9 | 6.82E−10 | 3.64E−09 | 1.69E−49 | 1.37E−10 |
| 10 | 5.59E−03 | 1.88E−03 | 1.12E−09 | 2.39E−05 |
| 11 | 9.17E−05 | 2.37E−04 | 1.28E−36 | 6.73E−06 |
| 12 | 8.04E−16 | 5.19E−14 | 7.03E−61 | 9.05E−21 |
| 13 | 5.55E−16 | 2.31E−14 | 2.47E−60 | 6.03E−21 |
| 14 | 5.30E−07 | 2.10E−06 | 1.38E−39 | 8.69E−08 |
| 15 | 1.67E−06 | 3.95E−06 | 1.23E−38 | 5.25E−08 |
| 16 | 4.67E−07 | 1.87E−06 | 6.39E−39 | 5.20E−08 |
| 17 | 4.17E−07 | 1.68E−06 | 2.84E−39 | 4.28E−08 |
| 18 | 5.95E−11 | 4.86E−10 | 4.79E−07 | 1.46E−09 |
| 19 | 4.65E−46 | 9.26E−45 | 3.20E−19 | 1.06E−36 |
| 20 | 5.55E−08 | 2.77E−08 | 8.59E−09 | 1.64E−09 |
| 21 | 4.89E−09 | 9.33E−07 | 3.24E−25 | 8.32E−08 |
| 22 | 8.50E−03 | 9.80E−03 | 5.28E−05 | 4.13E−03 |
| 23 | 4.82E−05 | 7.11E−05 | 5.83E−03 | 5.89E−04 |
| 24 | 1.31E−04 | 1.77E−04 | 7.59E−03 | 2.84E−03 |
| 25 | 2.32E−15 | 2.75E−17 | 6.79E−43 | 2.56E−16 |
| 26 | 2.32E−15 | 2.75E−17 | 6.79E−43 | 2.56E−16 |
| 27 | 3.66E−03 | 4.47E−03 | 4.75E−03 | 2.48E−03 |
| 28 | 6.25E−11 | 6.54E−10 | 4.02E−42 | 2.80E−12 |
| 29 | 6.60E−17 | 2.67E−17 | 8.45E−42 | 2.23E−17 |
| 30 | 7.29E−11 | 7.76E−10 | 1.57E−41 | 5.31E−12 |
| 31 | 7.91E−11 | 8.46E−10 | 2.18E−41 | 1.70E−11 |
| 32 | 4.29E−06 | 1.52E−06 | 1.28E−40 | 1.80E−07 |
| 33 | 1.31E−16 | 5.36E−17 | 3.96E−41 | 4.26E−17 |
| 34 | 1.31E−16 | 5.36E−17 | 3.96E−41 | 4.26E−17 |
| 35 | 1.31E−16 | 5.36E−17 | 3.96E−41 | 4.26E−17 |
| 36 | 1.43E−16 | 5.77E−17 | 4.56E−41 | 1.17E−17 |
| 37 | 6.46E−05 | 1.74E−04 | 2.25E−40 | 1.80E−06 |
| 38 | 2.55E−16 | 1.06E−16 | 5.76E−41 | 7.39E−17 |
| 39 | 2.58E−16 | 1.06E−16 | 1.05E−40 | 7.84E−17 |
| 40 | 2.68E−16 | 1.10E−16 | 1.19E−40 | 2.23E−17 |
| 41 | 2.68E−16 | 1.10E−16 | 1.19E−40 | 2.23E−17 |
| 42 | 2.68E−16 | 1.10E−16 | 1.19E−40 | 2.23E−17 |
| 43 | 1.90E−05 | 2.89E−05 | 6.00E−03 | 1.99E−04 |
| 44 | 1.84E−06 | 5.25E−07 | 1.05E−39 | 6.76E−08 |
| 45 | 8.60E−10 | 4.43E−09 | 9.49E−40 | 8.85E−14 |
| 46 | 2.36E−11 | 1.27E−10 | 4.92E−38 | 2.22E−11 |
| 47 | 1.07E−01 | 1.14E−01 | 1.50E−02 | 6.49E−02 |
| 48 | 4.02E−16 | 1.67E−16 | 1.34E−38 | 7.42E−18 |
| 49 | 4.46E−16 | 1.95E−16 | 3.53E−38 | 1.42E−17 |
| 50 | 4.97E−15 | 2.04E−15 | 7.83E−38 | 2.27E−17 |

TABLE 8f
Data for Escherichia, particularly E. coli (continued)

| No. | GM_pv_adj | LVX_pv_adj | TO_pv_adj | T/S_pv_adj |
|---|---|---|---|---|
| 1 | 2.40E−14 | 2.96E−16 | 6.56E−10 | 5.59E−55 |
| 2 | 9.02E−31 | 5.84E−43 | 3.02E−26 | 1.62E−119 |
| 3 | 3.09E−28 | 1.25E−33 | 1.52E−27 | 6.25E−97 |
| 4 | 1.19E−27 | 5.56E−32 | 5.30E−27 | 8.78E−96 |
| 5 | 2.33E−13 | 1.57E−38 | 6.37E−15 | 7.68E−72 |
| 6 | 4.17E−11 | 5.22E−11 | 1.79E−08 | 1.58E−71 |
| 7 | 7.23E−11 | 6.17E−14 | 8.00E−05 | 1.01E−69 |
| 8 | 2.80E−10 | 1.39E−13 | 8.00E−05 | 1.23E−68 |
| 9 | 1.18E−18 | 2.49E−49 | 2.67E−22 | 6.77E−68 |
| 10 | 7.65E−07 | 1.10E−08 | 7.07E−03 | 1.95E−64 |
| 11 | 1.21E−16 | 1.92E−37 | 4.13E−18 | 1.87E−63 |
| 12 | 4.86E−26 | 7.43E−62 | 5.25E−32 | 2.58E−39 |
| 13 | 2.72E−26 | 3.08E−61 | 2.92E−32 | 4.30E−39 |
| 14 | 1.39E−16 | 1.68E−39 | 1.59E−17 | 1.74E−60 |
| 15 | 5.78E−18 | 2.01E−39 | 1.91E−19 | 2.44E−60 |
| 16 | 1.02E−16 | 7.87E−39 | 1.17E−17 | 6.74E−60 |
| 17 | 7.38E−17 | 3.56E−39 | 8.56E−18 | 2.63E−59 |
| 18 | 6.73E−07 | 9.82E−07 | 5.94E−07 | 3.47E−47 |
| 19 | 5.34E−12 | 1.45E−19 | 7.00E−09 | 2.74E−06 |
| 20 | 3.89E−07 | 1.14E−08 | 2.09E−05 | 7.71E−46 |
| 21 | 6.04E−13 | 2.10E−24 | 3.47E−12 | 1.02E−37 |
| 22 | 8.52E−06 | 1.56E−04 | 7.54E−03 | 4.90E−44 |
| 23 | 9.39E−05 | 9.80E−03 | 1.28E−02 | 6.98E−44 |
| 24 | 2.16E−04 | 1.65E−02 | 9.49E−03 | 9.68E−44 |
| 25 | 1.81E−12 | 3.58E−43 | 2.05E−13 | 2.13E−06 |
| 26 | 1.81E−12 | 3.58E−43 | 2.05E−13 | 2.13E−06 |
| 27 | 1.08E−04 | 8.15E−03 | 5.44E−02 | 5.54E−43 |
| 28 | 1.81E−12 | 2.71E−42 | 4.67E−11 | 6.64E−17 |
| 29 | 3.65E−19 | 9.37E−42 | 2.80E−17 | 3.20E−18 |
| 30 | 3.61E−12 | 1.03E−41 | 9.37E−11 | 2.18E−16 |
| 31 | 3.86E−12 | 1.47E−41 | 9.73E−11 | 2.45E−16 |
| 32 | 1.03E−13 | 2.44E−41 | 2.22E−16 | 4.82E−04 |
| 33 | 4.79E−19 | 2.61E−41 | 5.83E−17 | 9.95E−18 |
| 34 | 4.79E−19 | 2.61E−41 | 5.83E−17 | 3.03E−17 |
| 35 | 4.79E−19 | 2.61E−41 | 5.83E−17 | 3.03E−17 |
| 36 | 8.47E−19 | 3.21E−41 | 6.04E−17 | 3.36E−18 |
| 37 | 3.56E−12 | 3.69E−41 | 8.59E−15 | 1.04E−04 |
| 38 | 8.74E−19 | 6.20E−41 | 6.42E−17 | 5.21E−17 |
| 39 | 9.35E−19 | 6.76E−41 | 6.96E−17 | 8.31E−17 |
| 40 | 1.03E−18 | 8.13E−41 | 1.27E−16 | 2.95E−17 |
| 41 | 1.03E−18 | 8.13E−41 | 1.27E−16 | 2.95E−17 |
| 42 | 1.03E−18 | 8.13E−41 | 1.27E−16 | 2.95E−17 |
| 43 | 9.79E−05 | 1.29E−02 | 1.33E−02 | 1.07E−40 |
| 44 | 1.98E−13 | 1.85E−40 | 4.73E−16 | 4.17E−04 |
| 45 | 2.68E−16 | 1.90E−40 | 1.10E−17 | 2.80E−08 |
| 46 | 1.27E−13 | 3.36E−40 | 7.14E−14 | 3.41E−07 |
| 47 | 1.15E−02 | 2.48E−02 | 4.44E−01 | 3.77E−40 |
| 48 | 1.50E−18 | 8.68E−39 | 9.76E−17 | 2.56E−15 |
| 49 | 3.08E−18 | 2.28E−38 | 1.98E−16 | 1.91E−14 |
| 50 | 2.46E−16 | 4.92E−38 | 8.76E−15 | 4.31E−13 |

TABLE 8g
Data for Escherichia, particularly E. coli (continued)

| No. | Locus.Tag | Gene.Symbol | GenBank.Accession | Chromosome |
|---|---|---|---|---|
| 1 | SeHA_C1580 | blaT | YP_002045448 | |
| 2 | EcolB7_01001258 | | ZP_00717086 | |
| 3 | HMPREF9345_05066 | | ZP_07100147 | |
| 4 | KPN_pKPN5p08201 | | YP_001338811 | plasmid pKPN5 |
| 5 | HMPREF9345_05232 | | ZP_07100311 | |
| 6 | EcolB_01004576 | | ZP_00708527 | |
| 7 | pEFER_0049 | | YP_002394596 | plasmid pEFER |
| 8 | ETAE_p040 | | YP_003297634 | plasmid pEIB202 |
| 9 | ECGG_04429 | | ZP_06648541 | |
| 10 | ETAE_p041 | folP | YP_003297635 | plasmid pEIB202 |
| 11 | ECGG_04434 | | ZP_06648545 | |
| 12 | ROD_p1051 | pemI | YP_003368450 | plasmid pCROD1 |
| 13 | ROD_p1061 | pemK | YP_003368451 | plasmid pCROD1 |
| 14 | HMPREF9348_05540 | | ZP_07692671 | |
| 15 | ECUMN_4816 | aadA | YP_002415408 | |
| 16 | ECUMN_4827 | mphA | YP_002415417 | |
| 17 | HMPREF9345_05223 | | ZP_07100302 | |
| 18 | EcSMS35_A0150 | | YP_001740030 | plasmid pSMS35_130 |
| 19 | HMPREF9548_03778 | | ZP_07141582 | |
| 20 | HMPREF9348_05561 | | ZP_07692692 | |
| 21 | HMPREF9548_03784 | | ZP_07141588 | |
| 22 | KPN_pKPN4p07064 | | YP_001338673 | plasmid pKPN4 |
| 23 | HMPREF9345_03525 | | ZP_07098656 | |
| 24 | KPN_pKPN4p07068 | merC | YP_001338677 | plasmid pKPN4 |
| 25 | ESCG_01010 | | ZP_04871321 | |
| 26 | HMPREF9540_00466 | | ZP_07133313 | |
| 27 | STMDT12_C39340 | | BAJ38877 | |
| 28 | KPK_A0022 | sopB | YP_002235674 | plasmid pKP187 |
| 29 | EcoliO157_010100014809 | | ZP_02780941 | |
| 30 | EcSMS35_A0072 | sopB | YP_001739960 | plasmid pSMS35_130 |
| 31 | HMPREF9538_00090 | | ZP_08302456 | |
| 32 | ECNA114_2444 | | AEG37278 | |
| 33 | EschericcoliO157_010100006568 | | ZP_02749755 | |
| 34 | CSAG_00128 | | ZP_04560798 | |
| 35 | ECH74115_0422 | mhpA | YP_002268988 | |
| 36 | CIT292_01335 | | ZP_03835561 | |
| 37 | ECSF_1165 | | BAI54705 | |
| 38 | EscherichiacoliO157EcO_010100009454 | | ZP_05948572 | |
| 39 | SSON_0329 | mhpD | YP_309341 | |
| 40 | CIT292_01336 | | ZP_03835562 | |
| 41 | HMPREF9540_00984 | | ZP_07133822 | |
| 42 | Missing | mhpE | AP_001004 | |
| | locus_tag:AC_000091.cds340.373092..374105 | | | |
| 43 | HMPREF9551_04611 | | ZP_07191964 | |
| 44 | SeHA_C2638 | | YP_002046450 | |
| 45 | SFxv_0758 | | ADA73054 | |
| 46 | HMPREF9530_01431 | | ZP_07151340 | |
| 47 | KPN_pKPN4p07069 | merA | YP_001338678 | plasmid pKPN4 |
| 48 | CP0077 | | NP_858210 | plasmid pCP301 |
| 49 | SDY_P083 | | YP_406084 | plasmid pSD1_197 |
| 50 | HMPREF9540_03398 | | ZP_07136184 | |

TABLE 8h
Data for Escherichia, particularly E. coli (continued)

| No. | Start.Coord | End.Coord | Strand | Scaffold.ID |
|---|---|---|---|---|
| 1 | 1525960 | 1526820 | + | 642555218 |
| 2 | 77515 | 77910 | + | 638358454 |
| 3 | 4715 | 5554 | − | 648294624 |
| 4 | 34908 | 35255 | − | 640753021 |
| 5 | 5252 | 5572 | − | 648294635 |
| 6 | 3555 | 4568 | + | 638359041 |
| 7 | 48658 | 49503 | + | 643692014 |
| 8 | 31237 | 32040 | − | 646311959 |
| 9 | 19278 | 19826 | + | 647536609 |
| 10 | 32101 | 32916 | − | 646311959 |
| 11 | 22618 | 23823 | + | 647536609 |
| 12 | 3339 | 3596 | + | 646311988 |
| 13 | 3529 | 3930 | + | 646311988 |
| 14 | 461 | 1045 | − | 649994953 |
| 15 | 4994882 | 4995670 | + | 644736335 |
| 16 | 5003053 | 5003958 | − | 644736335 |
| 17 | 1167 | 2405 | + | 648294635 |
| 18 | 118542 | 119474 | + | 641522668 |
| 19 | 4493 | 5755 | + | 648288593 |
| 20 | 209 | 661 | + | 649994959 |
| 21 | 8819 | 9535 | + | 648288593 |
| 22 | 23310 | 24614 | − | 640753020 |
| 23 | 1071 | 1307 | − | 648294596 |
| 24 | 25970 | 27664 | − | 640753020 |
| 25 | 241682 | 242032 | + | 646206991 |
| 26 | 192 | 443 | + | 648287910 |
| 27 | 4084530 | 4085237 | − | 651053007 |
| 28 | 12106 | 13071 | − | 643348532 |
| 29 | 123136 | 123969 | − | 641781714 |
| 30 | 53664 | 54635 | − | 641522668 |
| 31 | 5657 | 6832 | + | 651332930 |
| 32 | 2476906 | 2477910 | − | 651053098 |
| 33 | 369676 | 370620 | + | 641780393 |
| 34 | 146099 | 147763 | + | 646206732 |
| 35 | 430810 | 432474 | + | 643348548 |
| 36 | 25505 | 26449 | + | 643891503 |
| 37 | 1248345 | 1249289 | + | 646862305 |
| 38 | 20015 | 20965 | + | 645964942 |
| 39 | 356486 | 357301 | + | 640427102 |
| 40 | 26463 | 27344 | + | 643891503 |
| 41 | 7204 | 8217 | + | 648287942 |
| 42 | 373092 | 374105 | + | 637000000 |
| 43 | 97 | 531 | − | 648293053 |
| 44 | 2550781 | 2552010 | + | 642555218 |
| 45 | 734541 | 734921 | + | 646862363 |
| 46 | 8 | 643 | − | 648290358 |
| 47 | 27716 | 28177 | − | 640753020 |
| 48 | 61397 | 61579 | + | 637000224 |
| 49 | 69343 | 69615 | − | 640427105 |
| 50 | 3415 | 4356 | − | 648288139 |

### Examples 5 and 6

### Klebsiella, particularly K. oxytoca and K. pneumoniae

The procedure was carried out as in Example 1, except that the following microorganisms were used:

Bacterial Strains

The inventors selected 1568 Klebsiella strains, particularly 1179 for Klebsiella pneumonia and 389 for Klebsiella oxytoca, from the microbiology strain collection at Siemens Healthcare Diagnostics (West Sacramento, Calif.) for susceptibility testing and whole genome sequencing.

From MetaRef, 1640 centroids of Klebsiella species were used as reference sequences for K. oxytoca, and 1641 centroids of K. pneumoniae were uses as reference sequences of K. pneumoniae.

The results for K. oxytoca are shown in Tables 9 (corresponding to Table 1) and 10 (corresponding to Table 2), and the results for K. pneumonia are shown in Tables 11 (corresponding to Table 1) and 12 (corresponding to Table 2).

TABLE 9a
Data for Klebsiella, particularly K. oxytoca

| No. | Scaffold.External.Accession | Scaffold.Name | best_pv |
|---|---|---|---|
| 1 | NC_011283 | Klebsiella pneumoniae 342: NC_011283 | 7.24545119859105e−19 |
| 2 | NZ_GG745508 | Klebsiella sp. 1_1_55 genomic scaffold supercont1.1: | 7.24545119859105e−19 |
| | | NZ_GG745508 | |
| 3 | NC_011283 | Klebsiella pneumoniae 342: NC_011283 | 1.17347446332913e−17 |
| 4 | NC_015663 | Enterobacter aerogenes KCTC 2190 chromosome: NC_015663 | 9.82145388713083e−17 |
| 5 | NC_013850 | Klebsiella variicola At-22 chromosome: NC_013850 | 4.06768464186916e−16 |
| 6 | NZ_ACZD01000230 | Klebsiella pneumoniae subsp. rhinoscleromatis ATCC 13884 | 4.06768464186916e−16 |
| | | contig00265: NZ_ACZD01000230 | |
| 7 | NC_013850 | Klebsiella variicola At-22 chromosome: NC_013850 | 6.05465029985694e−16 |
| 8 | CP002910 | Klebsiella pneumoniae KCTC 2242: CP002910 | 6.05465029985694e−16 |
| 9 | CP002910 | Klebsiella pneumoniae KCTC 2242: CP002910 | 6.05465029985694e−16 |
| 10 | NZ_GG745509 | Klebsiella sp. 1_1_55 genomic scaffold supercont1.2: | 6.05465029985694e−16 |
| | | NZ_GG745509 | |
| 11 | NZ_GG745508 | Klebsiella sp. 1_1_55 genomic scaffold supercont1.1: | 6.05465029985694e−16 |
| | | NZ_GG745508 | |
| 12 | NC_011283 | Klebsiella pneumoniae 342: NC_011283 | 6.05465029985694e−16 |
| 13 | NC_013850 | Klebsiella variicola At-22 chromosome: NC_013850 | 6.05465029985694e−16 |
| 14 | NC_013850 | Klebsiella variicola At-22 chromosome: NC_013850 | 6.05465029985694e−16 |
| 15 | NC_013850 | Klebsiella variicola At-22 chromosome: NC_013850 | 6.05465029985694e−16 |
| 16 | NC_013850 | Klebsiella variicola At-22 chromosome: NC_013850 | 6.05465029985694e−16 |
| 17 | NC_013850 | Klebsiella variicola At-22 chromosome: NC_013850 | 6.05465029985694e−16 |
| 18 | NZ_GG745512 | Klebsiella sp. 1_1_55 genomic scaffold supercont1.5: | 6.05465029985694e−16 |
| | | NZ_GG745512 | |
| 19 | NC_013850 | Klebsiella variicola At-22 chromosome: NC_013850 | 6.05465029985694e−16 |
| 20 | NC_013850 | Klebsiella variicola At-22 chromosome: NC_013850 | 6.05465029985694e−16 |
| 21 | NZ_GG745508 | Klebsiella sp. 1_1_55 genomic scaffold supercont1.1: | 6.05465029985694e−16 |
| | | NZ_GG745508 | |
| 22 | NC_009648 | Klebsiella pneumoniae subsp. pneumoniae MGH 78578: | 6.05465029985694e−16 |
| | | NC_009648 | |
| 23 | NC_013850 | Klebsiella variicola At-22 chromosome: NC_013850 | 6.05465029985694e−16 |
| 24 | NC_013850 | Klebsiella variicola At-22 chromosome: NC_013850 | 6.05465029985694e−16 |
| 25 | NC_012731 | Klebsiella pneumoniae NTUH-K2044: NC_012731 | 6.05465029985694e−16 |
| 26 | NC_013850 | Klebsiella variicola At-22 chromosome: NC_013850 | 6.05465029985694e−16 |
| 27 | NC_013850 | Klebsiella variicola At-22 chromosome: NC_013850 | 6.05465029985694e−16 |
| 28 | NZ_GG745508 | Klebsiella sp. 1_1_55 genomic scaffold supercont1.1: | 6.05465029985694e−16 |
| | | NZ_GG745508 | |
| 29 | NC_013850 | Klebsiella variicola At-22 chromosome: NC_013850 | 6.05465029985694e−16 |
| 30 | NC_013850 | Klebsiella variicola At-22 chromosome: NC_013850 | 6.05465029985694e−16 |
| 31 | NC_013850 | Klebsiella variicola At-22 chromosome: NC_013850 | 6.05465029985694e−16 |
| 32 | NZ_GG745508 | Klebsiella sp. 1_1_55 genomic scaffold supercont1.1: | 6.05465029985694e−16 |
| | | NZ_GG745508 | |
| 33 | NZ_GG745508 | Klebsiella sp. 1_1_55 genomic scaffold supercont1.1: | 6.05465029985694e−16 |
| | | NZ_GG745508 | |
| 34 | NC_012731 | Klebsiella pneumoniae NTUH-K2044: NC_012731 | 6.05465029985694e−16 |
| 35 | CP002910 | Klebsiella pneumoniae KCTC 2242: CP002910 | 6.05465029985694e−16 |
| 36 | NC_011283 | Klebsiella pneumoniae 342: NC_011283 | 6.05465029985694e−16 |
| 37 | NZ_AFBO01000503 | Klebsiella sp. MS 92-3 K_spMS92-3-1.0_Cont988.13: | 6.05465029985694e−16 |
| | | NZ_AFBO01000503 | |
| 38 | NZ_GG745509 | Klebsiella sp. 1_1_55 genomic scaffold supercont1.2: | 6.05465029985694e−16 |
| | | NZ_GG745509 | |
| 39 | NZ_GG745509 | Klebsiella sp. 1_1_55 genomic scaffold supercont1.2: | 6.05465029985694e−16 |
| | | NZ_GG745509 | |
| 40 | NC_013850 | Klebsiella variicola At-22 chromosome: NC_013850 | 6.05465029985694e−16 |
| 41 | NC_013850 | Klebsiella variicola At-22 chromosome: NC_013850 | 6.05465029985694e−16 |
| 42 | NC_013850 | Klebsiella variicola At-22 chromosome: NC_013850 | 6.05465029985694e−16 |
| 43 | NC_009648 | Klebsiella pneumoniae subsp. pneumoniae MGH 78578: | 6.05465029985694e−16 |
| | | NC_009648 | |
| 44 | NZ_GG745509 | Klebsiella sp. 1_1_55 genomic scaffold supercont1.2: | 6.05465029985694e−16 |
| | | NZ_GG745509 | |
| 45 | NC_013850 | Klebsiella variicola At-22 chromosome: NC_013850 | 6.05465029985694e−16 |
| 46 | NC_013850 | Klebsiella variicola At-22 chromosome: NC_013850 | 6.05465029985694e−16 |
| 47 | NZ_AFBO01000685 | Klebsiella sp. MS 92-3 K_spMS92-3-1.0_Cont1649.3: | 6.05465029985694e−16 |
| | | NZ_AFBO01000685 | |
| 48 | CP002910 | Klebsiella pneumoniae KCTC 2242: CP002910 | 6.05465029985694e−16 |
| 49 | NC_013850 | Klebsiella variicola At-22 chromosome: NC_013850 | 6.05465029985694e−16 |
| 50 | NC_013850 | Klebsiella variicola At-22 chromosome: NC_013850 | 6.05465029985694e−16 |

TABLE 9b
Data for Klebsiella, particularly K. oxytoca (continued)

| No. | sign_phenos | sign_phenos_class | best_pheno | best_pheno_class |
|---|---|---|---|---|
| 1 | A/S; AUG; CP; CRM; LVX; P/T | fluoroquinolone; lactam | A/S | lactam |
| 2 | A/S; AUG; CP; CRM; LVX; P/T | fluoroquinolone; lactam | A/S | lactam |
| 3 | A/S; AUG; CRM; LVX; P/T | fluoroquinolone; lactam | A/S | lactam |
| 4 | A/S; AUG; CP; CRM; LVX; P/T | fluoroquinolone; lactam | A/S | lactam |
| 5 | A/S; CP; LVX | fluoroquinolone; lactam | A/S | lactam |
| 6 | A/S; AUG; CP; CRM; LVX; P/T | fluoroquinolone; lactam | A/S | lactam |
| 7 | A/S; AUG; CP; CRM; LVX | fluoroquinolone; lactam | A/S | lactam |
| 8 | A/S; AUG; CP; CRM; LVX | fluoroquinolone; lactam | A/S | lactam |
| 9 | A/S; CRM; LVX | fluoroquinolone; lactam | A/S | lactam |
| 10 | A/S; AUG; CP; LVX | fluoroquinolone; lactam | A/S | lactam |
| 11 | A/S; AUG; CP; CRM; LVX | fluoroquinolone; lactam | A/S | lactam |
| 12 | A/S; AUG; CP; CRM; LVX | fluoroquinolone; lactam | A/S | lactam |
| 13 | A/S; AUG; CP; CRM; LVX | fluoroquinolone; lactam | A/S | lactam |
| 14 | A/S; AUG; CP; CRM; LVX | fluoroquinolone; lactam | A/S | lactam |
| 15 | A/S; CP; LVX | fluoroquinolone; lactam | A/S | lactam |
| 16 | A/S; AUG; CP; CRM; LVX; P/T | fluoroquinolone; lactam | A/S | lactam |
| 17 | A/S; AUG; CP; CRM; LVX | fluoroquinolone; lactam | A/S | lactam |
| 18 | A/S; AUG; CP; CRM; LVX | fluoroquinolone; lactam | A/S | lactam |
| 19 | A/S; AUG; CP; CRM; LVX | fluoroquinolone; lactam | A/S | lactam |
| 20 | A/S; AUG; CP; CRM; LVX | fluoroquinolone; lactam | A/S | lactam |
| 21 | A/S; AUG; CP; CRM; LVX | fluoroquinolone; lactam | A/S | lactam |
| 22 | A/S; AUG; CP; CRM; LVX | fluoroquinolone; lactam | A/S | lactam |
| 23 | A/S; AUG; CP; CRM; LVX | fluoroquinolone; lactam | A/S | lactam |
| 24 | A/S; AUG; CP; CRM; LVX | fluoroquinolone; lactam | A/S | lactam |
| 25 | A/S; AUG; CP; CRM; LVX | fluoroquinolone; lactam | A/S | lactam |
| 26 | A/S; AUG; CP; CRM; LVX | fluoroquinolone; lactam | A/S | lactam |
| 27 | A/S; AUG; CP; CRM; LVX | fluoroquinolone; lactam | A/S | lactam |
| 28 | A/S; AUG; CP; CRM; LVX | fluoroquinolone; lactam | A/S | lactam |
| 29 | A/S; AUG; CP; CRM; LVX | fluoroquinolone; lactam | A/S | lactam |
| 30 | A/S; AUG; CP; CRM; LVX | fluoroquinolone; lactam | A/S | lactam |
| 31 | A/S; AUG; CP; CRM; LVX | fluoroquinolone; lactam | A/S | lactam |
| 32 | A/S; AUG; CP; CRM; LVX | fluoroquinolone; lactam | A/S | lactam |
| 33 | A/S; AUG; CP; CRM; LVX | fluoroquinolone; lactam | A/S | lactam |
| 34 | A/S; AUG; CP; CRM; LVX | fluoroquinolone; lactam | A/S | lactam |
| 35 | A/S; AUG; CP; CRM; LVX | fluoroquinolone; lactam | A/S | lactam |
| 36 | A/S; AUG; CP; CRM; LVX | fluoroquinolone; lactam | A/S | lactam |
| 37 | A/S; AUG; CP; CRM; LVX | fluoroquinolone; lactam | A/S | lactam |
| 38 | A/S; AUG; CP; CRM; LVX | fluoroquinolone; lactam | A/S | lactam |
| 39 | A/S; AUG; CP; CRM; LVX | fluoroquinolone; lactam | A/S | lactam |
| 40 | A/S; CRM; LVX | fluoroquinolone; lactam | A/S | lactam |
| 41 | A/S; AUG; CP; CRM; LVX | fluoroquinolone; lactam | A/S | lactam |
| 42 | A/S; AUG; CP; CRM; LVX | fluoroquinolone; lactam | A/S | lactam |
| 43 | A/S; AUG; CP; CRM; LVX | fluoroquinolone; lactam | A/S | lactam |
| 44 | A/S; CRM; LVX | fluoroquinolone; lactam | A/S | lactam |
| 45 | A/S; AUG; CP; CRM; LVX | fluoroquinolone; lactam | A/S | lactam |
| 46 | A/S; AUG; CP; CRM; LVX | fluoroquinolone; lactam | A/S | lactam |
| 47 | A/S; CP; LVX | fluoroquinolone; lactam | A/S | lactam |
| 48 | A/S; CRM; LVX | fluoroquinolone; lactam | A/S | lactam |
| 49 | A/S; AUG; CP; CRM; LVX | fluoroquinolone; lactam | A/S | lactam |
| 50 | A/S; CRM; LVX | fluoroquinolone; lactam | A/S | lactam |

TABLE 9c
Data for Klebsiella, particularly K. oxytoca (continued)

| No. | num_fluoroquinolone | num_lactam |
|---|---|---|
| 1 | 2 | 4 |
| 2 | 2 | 4 |
| 3 | 1 | 4 |
| 4 | 2 | 4 |
| 5 | 2 | 1 |
| 6 | 2 | 4 |
| 7 | 2 | 3 |
| 8 | 2 | 3 |
| 9 | 1 | 2 |
| 10 | 2 | 2 |
| 11 | 2 | 3 |
| 12 | 2 | 3 |
| 13 | 2 | 3 |
| 14 | 2 | 3 |
| 15 | 2 | 1 |
| 16 | 2 | 4 |
| 17 | 2 | 3 |
| 18 | 2 | 3 |
| 19 | 2 | 3 |
| 20 | 2 | 3 |
| 21 | 2 | 3 |
| 22 | 2 | 3 |
| 23 | 2 | 3 |
| 24 | 2 | 3 |
| 25 | 2 | 3 |
| 26 | 2 | 3 |
| 27 | 2 | 3 |
| 28 | 2 | 3 |
| 29 | 2 | 3 |
| 30 | 2 | 3 |
| 31 | 2 | 3 |
| 32 | 2 | 3 |
| 33 | 2 | 3 |
| 34 | 2 | 3 |
| 35 | 2 | 3 |
| 36 | 2 | 3 |
| 37 | 2 | 3 |
| 38 | 2 | 3 |
| 39 | 2 | 3 |
| 40 | 1 | 2 |
| 41 | 2 | 3 |
| 42 | 2 | 3 |
| 43 | 2 | 3 |
| 44 | 1 | 2 |
| 45 | 2 | 3 |
| 46 | 2 | 3 |
| 47 | 2 | 1 |
| 48 | 1 | 2 |
| 49 | 2 | 3 |
| 50 | 1 | 2 |

TABLE 9d
Data for Klebsiella, particularly K. oxytoca (continued)

| No. | A/S_pv_adj | AUG_pv_adj | AZT_pv_adj | CAX_pv_adj | CFT_pv_adj |
|---|---|---|---|---|---|
| 1 | 7.25E−19 | 1.72E−03 | 1.02E−01 | 7.63E−02 | 1.14E−01 |
| 2 | 7.25E−19 | 1.72E−03 | 1.02E−01 | 7.63E−02 | 1.14E−01 |
| 3 | 1.17E−17 | 2.11E−03 | 1.25E−02 | 1.67E−02 | 3.76E−02 |
| 4 | 9.82E−17 | 1.48E−03 | 1.97E−02 | 1.37E−02 | 6.22E−02 |
| 5 | 4.07E−16 | 1.01E−02 | 2.64E−02 | 3.46E−02 | 3.40E−02 |
| 6 | 4.07E−16 | 2.92E−04 | 3.51E−02 | 2.48E−02 | 4.69E−02 |
| 7 | 6.05E−16 | 7.34E−03 | 3.46E−02 | 2.50E−02 | 2.46E−02 |
| 8 | 6.05E−16 | 7.34E−03 | 3.46E−02 | 2.50E−02 | 2.46E−02 |
| 9 | 6.05E−16 | 1.93E−02 | 7.20E−02 | 7.25E−02 | 5.85E−02 |
| 10 | 6.05E−16 | 9.84E−03 | 4.51E−02 | 3.38E−02 | 2.50E−02 |
| 11 | 6.05E−16 | 7.34E−03 | 3.46E−02 | 2.50E−02 | 2.46E−02 |
| 12 | 6.05E−16 | 7.34E−03 | 3.46E−02 | 2.50E−02 | 2.46E−02 |
| 13 | 6.05E−16 | 7.34E−03 | 3.46E−02 | 2.50E−02 | 2.46E−02 |
| 14 | 6.05E−16 | 7.34E−03 | 3.46E−02 | 2.50E−02 | 2.46E−02 |
| 15 | 6.05E−16 | 1.01E−02 | 4.61E−02 | 3.45E−02 | 3.46E−02 |
| 16 | 6.05E−16 | 5.13E−03 | 2.58E−02 | 1.89E−02 | 1.75E−02 |
| 17 | 6.05E−16 | 7.34E−03 | 3.46E−02 | 2.50E−02 | 2.46E−02 |
| 18 | 6.05E−16 | 7.34E−03 | 3.46E−02 | 2.50E−02 | 2.46E−02 |
| 19 | 6.05E−16 | 7.34E−03 | 3.46E−02 | 2.50E−02 | 2.46E−02 |
| 20 | 6.05E−16 | 7.34E−03 | 3.46E−02 | 2.50E−02 | 2.46E−02 |
| 21 | 6.05E−16 | 7.34E−03 | 3.46E−02 | 2.50E−02 | 2.46E−02 |
| 22 | 6.05E−16 | 7.34E−03 | 3.46E−02 | 2.50E−02 | 2.46E−02 |
| 23 | 6.05E−16 | 7.34E−03 | 3.46E−02 | 2.50E−02 | 2.46E−02 |
| 24 | 6.05E−16 | 7.34E−03 | 3.46E−02 | 2.50E−02 | 2.46E−02 |
| 25 | 6.05E−16 | 5.42E−03 | 3.46E−02 | 1.98E−02 | 2.46E−02 |
| 26 | 6.05E−16 | 7.34E−03 | 3.46E−02 | 2.50E−02 | 2.46E−02 |
| 27 | 6.05E−16 | 7.34E−03 | 3.46E−02 | 2.50E−02 | 2.46E−02 |
| 28 | 6.05E−16 | 7.34E−03 | 3.46E−02 | 2.50E−02 | 2.46E−02 |
| 29 | 6.05E−16 | 7.34E−03 | 3.46E−02 | 2.50E−02 | 2.46E−02 |
| 30 | 6.05E−16 | 7.34E−03 | 3.46E−02 | 2.50E−02 | 2.46E−02 |
| 31 | 6.05E−16 | 7.34E−03 | 3.46E−02 | 2.50E−02 | 2.46E−02 |
| 32 | 6.05E−16 | 7.34E−03 | 3.46E−02 | 2.50E−02 | 2.46E−02 |
| 33 | 6.05E−16 | 7.34E−03 | 3.46E−02 | 2.50E−02 | 2.46E−02 |
| 34 | 6.05E−16 | 7.34E−03 | 3.46E−02 | 2.50E−02 | 2.46E−02 |
| 35 | 6.05E−16 | 7.34E−03 | 3.46E−02 | 2.50E−02 | 2.46E−02 |
| 36 | 6.05E−16 | 7.34E−03 | 3.46E−02 | 2.50E−02 | 2.46E−02 |
| 37 | 6.05E−16 | 7.34E−03 | 3.46E−02 | 2.50E−02 | 2.46E−02 |
| 38 | 6.05E−16 | 7.34E−03 | 3.46E−02 | 2.50E−02 | 2.46E−02 |
| 39 | 6.05E−16 | 7.34E−03 | 3.46E−02 | 2.50E−02 | 2.46E−02 |
| 40 | 6.05E−16 | 1.96E−02 | 7.24E−02 | 9.30E−02 | 5.85E−02 |
| 41 | 6.05E−16 | 7.42E−03 | 3.51E−02 | 2.53E−02 | 2.48E−02 |
| 42 | 6.05E−16 | 7.34E−03 | 3.46E−02 | 2.50E−02 | 2.46E−02 |
| 43 | 6.05E−16 | 7.42E−03 | 3.51E−02 | 2.53E−02 | 2.48E−02 |
| 44 | 6.05E−16 | 1.96E−02 | 7.24E−02 | 9.30E−02 | 5.85E−02 |
| 45 | 6.05E−16 | 7.34E−03 | 3.46E−02 | 2.50E−02 | 2.46E−02 |
| 46 | 6.05E−16 | 7.42E−03 | 3.51E−02 | 2.53E−02 | 2.48E−02 |
| 47 | 6.05E−16 | 1.84E−02 | 7.73E−02 | 5.74E−02 | 4.41E−02 |
| 48 | 6.05E−16 | 1.96E−02 | 7.24E−02 | 9.30E−02 | 5.85E−02 |
| 49 | 6.05E−16 | 7.34E−03 | 3.46E−02 | 2.50E−02 | 2.46E−02 |
| 50 | 6.05E−16 | 1.96E−02 | 7.24E−02 | 9.30E−02 | 5.85E−02 |

TABLE 9e
Data for Klebsiella, particularly K. oxytoca (continued)

| No. | CP_pv_adj | CRM_pv_adj | LVX_pv_adj | P/T_pv_adj |
|---|---|---|---|---|
| 1 | 4.47E−03 | 3.22E−03 | 3.33E−05 | 4.21E−03 |
| 2 | 4.47E−03 | 3.22E−03 | 3.33E−05 | 4.21E−03 |
| 3 | 4.19E−02 | 3.50E−03 | 2.38E−03 | 7.73E−03 |
| 4 | 2.12E−04 | 4.16E−03 | 5.03E−07 | 3.38E−03 |
| 5 | 2.11E−03 | 1.38E−02 | 6.28E−05 | 1.93E−02 |
| 6 | 6.71E−04 | 1.62E−03 | 8.24E−07 | 2.85E−03 |
| 7 | 1.30E−03 | 7.85E−03 | 5.53E−05 | 1.37E−02 |
| 8 | 1.30E−03 | 7.85E−03 | 5.53E−05 | 1.37E−02 |
| 9 | 6.62E−02 | 2.94E−03 | 1.50E−03 | 3.78E−02 |
| 10 | 1.98E−03 | 1.03E−02 | 5.68E−05 | 1.89E−02 |
| 11 | 1.30E−03 | 7.85E−03 | 5.53E−05 | 1.37E−02 |
| 12 | 1.30E−03 | 7.85E−03 | 5.53E−05 | 1.37E−02 |
| 13 | 1.30E−03 | 7.85E−03 | 5.53E−05 | 1.37E−02 |
| 14 | 1.30E−03 | 7.85E−03 | 5.53E−05 | 1.37E−02 |
| 15 | 1.26E−03 | 1.04E−02 | 3.08E−05 | 1.93E−02 |
| 16 | 7.58E−04 | 6.15E−03 | 5.93E−05 | 9.93E−03 |
| 17 | 1.30E−03 | 7.85E−03 | 5.53E−05 | 1.37E−02 |
| 18 | 1.30E−03 | 7.85E−03 | 5.53E−05 | 1.37E−02 |
| 19 | 1.30E−03 | 7.85E−03 | 5.53E−05 | 1.37E−02 |
| 20 | 1.30E−03 | 7.85E−03 | 5.53E−05 | 1.37E−02 |
| 21 | 1.30E−03 | 7.85E−03 | 5.53E−05 | 1.37E−02 |
| 22 | 1.30E−03 | 7.85E−03 | 5.53E−05 | 1.37E−02 |
| 23 | 1.30E−03 | 7.85E−03 | 5.53E−05 | 1.37E−02 |
| 24 | 1.30E−03 | 7.85E−03 | 5.53E−05 | 1.37E−02 |
| 25 | 3.39E−03 | 6.15E−03 | 1.55E−04 | 1.03E−02 |
| 26 | 1.30E−03 | 7.85E−03 | 5.53E−05 | 1.37E−02 |
| 27 | 1.30E−03 | 7.85E−03 | 5.53E−05 | 1.37E−02 |
| 28 | 1.30E−03 | 7.85E−03 | 5.53E−05 | 1.37E−02 |
| 29 | 1.30E−03 | 7.85E−03 | 5.53E−05 | 1.37E−02 |
| 30 | 1.30E−03 | 7.85E−03 | 5.53E−05 | 1.37E−02 |
| 31 | 1.30E−03 | 7.85E−03 | 5.53E−05 | 1.37E−02 |
| 32 | 1.30E−03 | 7.85E−03 | 5.53E−05 | 1.37E−02 |
| 33 | 1.30E−03 | 7.85E−03 | 5.53E−05 | 1.37E−02 |
| 34 | 1.30E−03 | 7.85E−03 | 5.53E−05 | 1.37E−02 |
| 35 | 1.30E−03 | 7.85E−03 | 5.53E−05 | 1.37E−02 |
| 36 | 1.30E−03 | 7.85E−03 | 5.53E−05 | 1.37E−02 |
| 37 | 1.30E−03 | 7.85E−03 | 5.53E−05 | 1.37E−02 |
| 38 | 1.30E−03 | 7.85E−03 | 5.53E−05 | 1.37E−02 |
| 39 | 1.30E−03 | 7.85E−03 | 5.53E−05 | 1.37E−02 |
| 40 | 9.00E−02 | 4.08E−03 | 1.50E−03 | 4.97E−02 |
| 41 | 1.97E−03 | 7.93E−03 | 5.53E−05 | 1.39E−02 |
| 42 | 1.30E−03 | 7.85E−03 | 5.53E−05 | 1.37E−02 |
| 43 | 1.97E−03 | 7.93E−03 | 5.53E−05 | 1.39E−02 |
| 44 | 9.00E−02 | 4.08E−03 | 1.50E−03 | 4.97E−02 |
| 45 | 1.30E−03 | 7.85E−03 | 5.53E−05 | 1.37E−02 |
| 46 | 1.97E−03 | 7.93E−03 | 5.53E−05 | 1.39E−02 |
| 47 | 7.43E−03 | 1.01E−02 | 2.47E−04 | 3.31E−02 |
| 48 | 9.00E−02 | 4.08E−03 | 1.50E−03 | 4.97E−02 |
| 49 | 1.30E−03 | 7.85E−03 | 5.53E−05 | 1.37E−02 |
| 50 | 9.00E−02 | 4.08E−03 | 1.50E−03 | 4.97E−02 |

TABLE 9f
Data for Klebsiella, particularly K. oxytoca (continued)

| No. | Locus.Tag | Gene.Symbol | GenBank.Accession | Chromosome |
|---|---|---|---|---|
| 1 | KPK_2590 | | YP_002238421 | |
| 2 | HMPREF0485_01216 | | ZP_06548816 | |
| 3 | KPK_0292 | | YP_002236174 | |
| 4 | EAE_11985 | | YP_004592590 | |
| 5 | Kvar_2784 | | YP_003439702 | |
| 6 | HMPREF0484_4675 | add | ZP_06017656 | |
| 7 | Kvar_2381 | | YP_003439305 | |
| 8 | KPN2242_04295 | | AEJ96779 | |
| 9 | KPN2242_08680 | | AEJ97652 | |
| 10 | HMPREF0485_01916 | | ZP_06549516 | |
| 11 | HMPREF0485_01247 | | ZP_06548847 | |
| 12 | KPK_2622 | | YP_002238453 | |
| 13 | Kvar_2568 | | YP_003439490 | |
| 14 | Kvar_0816 | | YP_003437758 | |
| 15 | Kvar_4643 | | YP_003441547 | |
| 16 | Kvar_3771 | | YP_003440683 | |
| 17 | Kvar_1596 | | YP_003438533 | |
| 18 | HMPREF0485_03998 | | ZP_06551594 | |
| 19 | Kvar_2140 | | YP_003439069 | |
| 20 | Kvar_3380 | | YP_003440292 | |
| 21 | HMPREF0485_00446 | | ZP_06548046 | |
| 22 | KPN_00275 | | YP_001333958 | |
| 23 | Kvar_1589 | | YP_003438526 | |
| 24 | Kvar_1856 | | YP_003438789 | |
| 25 | KP1_3016 | | YP_002919724 | |
| 26 | Kvar_5099 | | YP_003442002 | |
| 27 | Kvar_0479 | | YP_003437421 | |
| 28 | HMPREF0485_00450 | | ZP_06548050 | |
| 29 | Kvar_1586 | | YP_003438523 | |
| 30 | Kvar_1598 | | YP_003438535 | |
| 31 | Kvar_1597 | | YP_003438534 | |
| 32 | HMPREF0485_00443 | | ZP_06548043 | |
| 33 | HMPREF0485_00453 | | ZP_06548053 | |
| 34 | KP1_2851 | | YP_002919569 | |
| 35 | KPN2242_01120 | | AEJ96149 | |
| 36 | KPK_2936 | | YP_002238762 | |
| 37 | HMPREF9538_03901 | | ZP_08306208 | |
| 38 | HMPREF0485_02477 | | ZP_06550077 | |
| 39 | HMPREF0485_01831 | | ZP_06549431 | |
| 40 | Kvar_3258 | | YP_003440171 | |
| 41 | Kvar_3395 | | YP_003440307 | |
| 42 | Kvar_3210 | | YP_003440127 | |
| 43 | KPN_00277 | | YP_001333960 | |
| 44 | HMPREF0485_02605 | | ZP_06550204 | |
| 45 | Kvar_1604 | | YP_003438541 | |
| 46 | Kvar_4545 | | YP_003441451 | |
| 47 | HMPREF9538_05113 | | ZP_08307402 | |
| 48 | KPN2242_08695 | | AEJ97655 | |
| 49 | Kvar_4742 | | YP_003441646 | |
| 50 | Kvar_3261 | | YP_003440174 | |

TABLE 9g
Data for Klebsiella, particularly K. oxytoca (continued)

| No. | Start.Coord | End.Coord | Strand | Scaffold.ID |
|---|---|---|---|---|
| 1 | 2648871 | 2649476 | + | 643348533 |
| 2 | 1355295 | 1356167 | − | 647536389 |
| 3 | 321413 | 321955 | − | 643348533 |
| 4 | 2576463 | 2577677 | − | 650716122 |
| 5 | 2889784 | 2890686 | − | 646312028 |
| 6 | 12390 | 13076 | + | 647009977 |
| 7 | 2489613 | 2491040 | + | 646312028 |
| 8 | 925860 | 926771 | − | 651053122 |
| 9 | 1837699 | 1838718 | + | 651053122 |
| 10 | 237063 | 237326 | + | 647536390 |
| 11 | 1387256 | 1388314 | + | 647536389 |
| 12 | 2680912 | 2681727 | + | 643348533 |
| 13 | 2679911 | 2680687 | + | 646312028 |
| 14 | 851698 | 852411 | + | 646312028 |
| 15 | 4934923 | 4936932 | + | 646312028 |
| 16 | 3964905 | 3966425 | + | 646312028 |
| 17 | 1721712 | 1722182 | − | 646312028 |
| 18 | 140912 | 141796 | + | 647536393 |
| 19 | 2255010 | 2255996 | + | 646312028 |
| 20 | 3519306 | 3520322 | − | 646312028 |
| 21 | 523538 | 524377 | − | 647536389 |
| 22 | 311624 | 312142 | + | 640753018 |
| 23 | 1716249 | 1716779 | + | 646312028 |
| 24 | 1971206 | 1973212 | − | 646312028 |
| 25 | 2878963 | 2879583 | + | 646564530 |
| 26 | 5455245 | 5456423 | − | 646312028 |
| 27 | 509672 | 510736 | + | 646312028 |
| 28 | 528513 | 529175 | − | 647536389 |
| 29 | 1711303 | 1712709 | − | 646312028 |
| 30 | 1723564 | 1724937 | − | 646312028 |
| 31 | 1722169 | 1723554 | − | 646312028 |
| 32 | 520902 | 521159 | − | 647536389 |
| 33 | 531062 | 532510 | − | 647536389 |
| 34 | 2717504 | 2718439 | + | 646564530 |
| 35 | 230663 | 231895 | − | 651053122 |
| 36 | 2983904 | 2984626 | + | 643348533 |
| 37 | 15395 | 16450 | + | 651333420 |
| 38 | 872347 | 873972 | + | 647536390 |
| 39 | 149922 | 150884 | − | 647536390 |
| 40 | 3387706 | 3389424 | − | 646312028 |
| 41 | 3535340 | 3535786 | + | 646312028 |
| 42 | 3340882 | 3341376 | − | 646312028 |
| 43 | 312969 | 315452 | + | 640753018 |
| 44 | 1009891 | 1010898 | + | 647536390 |
| 45 | 1730820 | 1734335 | + | 646312028 |
| 46 | 4822901 | 4823620 | + | 646312028 |
| 47 | 52 | 390 | − | 651333602 |
| 48 | 1839612 | 1840562 | + | 651053122 |
| 49 | 5039066 | 5040004 | + | 646312028 |
| 50 | 3391540 | 3392502 | + | 646312028 |

TABLE 10a
Data for Klebsiella, particularly K. oxytoca

| | Scaffold.External. | | |
|---|---|---|---|
| No. | Accession | Scaffold.Name | best_pv |
| 1 | NC_011283 | Klebsiella pneumoniae 342: NC_011283 | 7.24545119859105e−19 |
| 2 | NZ_GG745508 | Klebsiella sp. 1_1_55 genomic scaffold supercont1.1: NZ_GG745508 | 7.24545119859105e−19 |
| 3 | NC_015663 | Enterobacter aerogenes KCTC 2190 chromosome: NC_015663 | 9.82145388713083e−17 |
| 4 | NC_013850 | Klebsiella variicola At-22 chromosome: NC_013850 | 4.06768464186916e−16 |
| 5 | NZ_ACZD01000230 | Klebsiella pneumoniae subsp. rhinoscleromatis ATCC 13884 | 4.06768464186916e−16 |
| | | contig00265: NZ_ACZD01000230 | |
| 6 | NC_013850 | Klebsiella variicola At-22 chromosome: NC_013850 | 6.05465029985694e−16 |
| 7 | CP002910 | Klebsiella pneumoniae KCTC 2242: CP002910 | 6.05465029985694e−16 |
| 8 | NZ_GG745509 | Klebsiella sp. 1_1_55 genomic scaffold supercont1.2: NZ_GG745509 | 6.05465029985694e−16 |
| 9 | NZ_GG745508 | Klebsiella sp. 1_1_55 genomic scaffold supercont1.1: NZ_GG745508 | 6.05465029985694e−16 |
| 10 | NC_011283 | Klebsiella pneumoniae 342: NC_011283 | 6.05465029985694e−16 |
| 11 | NC_013850 | Klebsiella variicola At-22 chromosome: NC_013850 | 6.05465029985694e−16 |
| 12 | NC_013850 | Klebsiella variicola At-22 chromosome: NC_013850 | 6.05465029985694e−16 |
| 13 | NC_013850 | Klebsiella variicola At-22 chromosome: NC_013850 | 6.05465029985694e−16 |
| 14 | NC_013850 | Klebsiella variicola At-22 chromosome: NC_013850 | 6.05465029985694e−16 |
| 15 | NC_013850 | Klebsiella variicola At-22 chromosome: NC_013850 | 6.05465029985694e−16 |
| 16 | NZ_GG745512 | Klebsiella sp. 1_1_55 genomic scaffold supercont1.5: NZ_GG745512 | 6.05465029985694e−16 |
| 17 | NC_013850 | Klebsiella variicola At-22 chromosome: NC_013850 | 6.05465029985694e−16 |
| 18 | NC_013850 | Klebsiella variicola At-22 chromosome: NC_013850 | 6.05465029985694e−16 |
| 19 | NZ_GG745508 | Klebsiella sp. 1_1_55 genomic scaffold supercont1.1: NZ_GG745508 | 6.05465029985694e−16 |
| 20 | NC_009648 | Klebsiella pneumoniae subsp. pneumoniae MGH 78578: NC_009648 | 6.05465029985694e−16 |
| 21 | NC_013850 | Klebsiella variicola At-22 chromosome: NC_013850 | 6.05465029985694e−16 |
| 22 | NC_013850 | Klebsiella variicola At-22 chromosome: NC_013850 | 6.05465029985694e−16 |
| 23 | NC_012731 | Klebsiella pneumoniae NTUH-K2044: NC_012731 | 6.05465029985694e−16 |
| 24 | NC_013850 | Klebsiella variicola At-22 chromosome: NC_013850 | 6.05465029985694e−16 |
| 25 | NC_013850 | Klebsiella variicola At-22 chromosome: NC_013850 | 6.05465029985694e−16 |
| 26 | NZ_GG745508 | Klebsiella sp. 1_1_55 genomic scaffold supercont1.1: NZ_GG745508 | 6.05465029985694e−16 |
| 27 | NC_013850 | Klebsiella variicola At-22 chromosome: NC_013850 | 6.05465029985694e−16 |
| 28 | NC_013850 | Klebsiella variicola At-22 chromosome: NC_013850 | 6.05465029985694e−16 |
| 29 | NC_013850 | Klebsiella variicola At-22 chromosome: NC_013850 | 6.05465029985694e−16 |
| 30 | NZ_GG745508 | Klebsiella sp. 1_1_55 genomic scaffold supercont1.1: NZ_GG745508 | 6.05465029985694e−16 |
| 31 | NZ_GG745508 | Klebsiella sp. 1_1_55 genomic scaffold supercont1.1: NZ_GG745508 | 6.05465029985694e−16 |
| 32 | NC_012731 | Klebsiella pneumoniae NTUH-K2044: NC_012731 | 6.05465029985694e−16 |
| 33 | CP002910 | Klebsiella pneumoniae KCTC 2242: CP002910 | 6.05465029985694e−16 |
| 34 | NC_011283 | Klebsiella pneumoniae 342: NC_011283 | 6.05465029985694e−16 |
| 35 | NZ_AFBO01000503 | Klebsiella sp. MS 92-3 K_spMS92-3-1.0_Cont988.13: | 6.05465029985694e−16 |
| | | NZ_AFBO01000503 | |
| 36 | NZ_GG745509 | Klebsiella sp. 1_1_55 genomic scaffold supercont1.2: NZ_GG745509 | 6.05465029985694e−16 |
| 37 | NZ_GG745509 | Klebsiella sp. 1_1_55 genomic scaffold supercont1.2: NZ_GG745509 | 6.05465029985694e−16 |
| 38 | NC_013850 | Klebsiella variicola At-22 chromosome: NC_013850 | 6.05465029985694e−16 |
| 39 | NC_013850 | Klebsiella variicola At-22 chromosome: NC_013850 | 6.05465029985694e−16 |
| 40 | NC_009648 | Klebsiella pneumoniae subsp. pneumoniae MGH 78578: NC_009648 | 6.05465029985694e−16 |
| 41 | NC_013850 | Klebsiella variicola At-22 chromosome: NC_013850 | 6.05465029985694e−16 |
| 42 | NC_013850 | Klebsiella variicola At-22 chromosome: NC_013850 | 6.05465029985694e−16 |
| 43 | NZ_AFBO01000685 | Klebsiella sp. MS 92-3 K_spMS92-3-1.0_Cont1649.3: | 6.05465029985694e−16 |
| | | NZ_AFBO01000685 | |
| 44 | NC_013850 | Klebsiella variicola At-22 chromosome: NC_013850 | 6.05465029985694e−16 |
| 45 | NC_013850 | Klebsiella variicola At-22 chromosome: NC_013850 | 6.05465029985694e−16 |
| 46 | NC_013850 | Klebsiella variicola At-22 chromosome: NC_013850 | 6.05465029985694e−16 |
| 47 | NC_009648 | Klebsiella pneumoniae subsp. pneumoniae MGH 78578: NC_009648 | 6.05465029985694e−16 |
| 48 | NC_011283 | Klebsiella pneumoniae 342: NC_011283 | 6.05465029985694e−16 |
| 49 | NC_014500 | Dickeya dadantii 3937 chromosome: NC_014500 | 6.05465029985694e−16 |
| 50 | NC_011283 | Klebsiella pneumoniae 342: NC_011283 | 6.05465029985694e−16 |

TABLE 10b
Data for Klebsiella, particularly K. oxytoca (continued)

| No. | sign_phenos | sign_phenos_class | best_pheno | best_pheno_class |
|---|---|---|---|---|
| 1 | A/S; AUG; CP; CRM; LVX; P/T | fluoroquinolone; lactam | A/S | lactam |
| 2 | A/S; AUG; CP; CRM; LVX; P/T | fluoroquinolone; lactam | A/S | lactam |
| 3 | A/S; AUG; CP; CRM; LVX; P/T | fluoroquinolone; lactam | A/S | lactam |
| 4 | A/S; CP; LVX | fluoroquinolone; lactam | A/S | lactam |
| 5 | A/S; AUG; CP; CRM; LVX; P/T | fluoroquinolone; lactam | A/S | lactam |
| 6 | A/S; AUG; CP; CRM; LVX | fluoroquinolone; lactam | A/S | lactam |
| 7 | A/S; AUG; CP; CRM; LVX | fluoroquinolone; lactam | A/S | lactam |
| 8 | A/S; AUG; CP; LVX | fluoroquinolone; lactam | A/S | lactam |
| 9 | A/S; AUG; CP; CRM; LVX | fluoroquinolone; lactam | A/S | lactam |
| 10 | A/S; AUG; CP; CRM; LVX | fluoroquinolone; lactam | A/S | lactam |
| 11 | A/S; AUG; CP; CRM; LVX | fluoroquinolone; lactam | A/S | lactam |
| 12 | A/S; AUG; CP; CRM; LVX | fluoroquinolone; lactam | A/S | lactam |
| 13 | A/S; CP; LVX | fluoroquinolone; lactam | A/S | lactam |
| 14 | A/S; AUG; CP; CRM; LVX; P/T | fluoroquinolone; lactam | A/S | lactam |
| 15 | A/S; AUG; CP; CRM; LVX | fluoroquinolone; lactam | A/S | lactam |
| 16 | A/S; AUG; CP; CRM; LVX | fluoroquinolone; lactam | A/S | lactam |
| 17 | A/S; AUG; CP; CRM; LVX | fluoroquinolone; lactam | A/S | lactam |
| 18 | A/S; AUG; CP; CRM; LVX | fluoroquinolone; lactam | A/S | lactam |
| 19 | A/S; AUG; CP; CRM; LVX | fluoroquinolone; lactam | A/S | lactam |
| 20 | A/S; AUG; CP; CRM; LVX | fluoroquinolone; lactam | A/S | lactam |
| 21 | A/S; AUG; CP; CRM; LVX | fluoroquinolone; lactam | A/S | lactam |
| 22 | A/S; AUG; CP; CRM; LVX | fluoroquinolone; lactam | A/S | lactam |
| 23 | A/S; AUG; CP; CRM; LVX | fluoroquinolone; lactam | A/S | lactam |
| 24 | A/S; AUG; CP; CRM; LVX | fluoroquinolone; lactam | A/S | lactam |
| 25 | A/S; AUG; CP; CRM; LVX | fluoroquinolone; lactam | A/S | lactam |
| 26 | A/S; AUG; CP; CRM; LVX | fluoroquinolone; lactam | A/S | lactam |
| 27 | A/S; AUG; CP; CRM; LVX | fluoroquinolone; lactam | A/S | lactam |
| 28 | A/S; AUG; CP; CRM; LVX | fluoroquinolone; lactam | A/S | lactam |
| 29 | A/S; AUG; CP; CRM; LVX | fluoroquinolone; lactam | A/S | lactam |
| 30 | A/S; AUG; CP; CRM; LVX | fluoroquinolone; lactam | A/S | lactam |
| 31 | A/S; AUG; CP; CRM; LVX | fluoroquinolone; lactam | A/S | lactam |
| 32 | A/S; AUG; CP; CRM; LVX | fluoroquinolone; lactam | A/S | lactam |
| 33 | A/S; AUG; CP; CRM; LVX | fluoroquinolone; lactam | A/S | lactam |
| 34 | A/S; AUG; CP; CRM; LVX | fluoroquinolone; lactam | A/S | lactam |
| 35 | A/S; AUG; CP; CRM; LVX | fluoroquinolone; lactam | A/S | lactam |
| 36 | A/S; AUG; CP; CRM; LVX | fluoroquinolone; lactam | A/S | lactam |
| 37 | A/S; AUG; CP; CRM; LVX | fluoroquinolone; lactam | A/S | lactam |
| 38 | A/S; AUG; CP; CRM; LVX | fluoroquinolone; lactam | A/S | lactam |
| 39 | A/S; AUG; CP; CRM; LVX | fluoroquinolone; lactam | A/S | lactam |
| 40 | A/S; AUG; CP; CRM; LVX | fluoroquinolone; lactam | A/S | lactam |
| 41 | A/S; AUG; CP; CRM; LVX | fluoroquinolone; lactam | A/S | lactam |
| 42 | A/S; AUG; CP; CRM; LVX | fluoroquinolone; lactam | A/S | lactam |
| 43 | A/S; CP; LVX | fluoroquinolone; lactam | A/S | lactam |
| 44 | A/S; AUG; CP; CRM; LVX | fluoroquinolone; lactam | A/S | lactam |
| 45 | A/S; AUG; CP; CRM; LVX | fluoroquinolone; lactam | A/S | lactam |
| 46 | A/S; AUG; CP; CRM; LVX | fluoroquinolone; lactam | A/S | lactam |
| 47 | A/S; AUG; CP; CRM; LVX | fluoroquinolone; lactam | A/S | lactam |
| 48 | A/S; AUG; CP; CRM; LVX | fluoroquinolone; lactam | A/S | lactam |
| 49 | A/S; AUG; CP; CRM; LVX | fluoroquinolone; lactam | A/S | lactam |
| 50 | A/S; AUG; CP; CRM; LVX | fluoroquinolone; lactam | A/S | lactam |

TABLE 10c
Data for Klebsiella, particularly K. oxytoca (continued)

| No. | num_fluoroquinolone | num_lactam |
|---|---|---|
| 1 | 2 | 4 |
| 2 | 2 | 4 |
| 3 | 2 | 4 |
| 4 | 2 | 1 |
| 5 | 2 | 4 |
| 6 | 2 | 3 |
| 7 | 2 | 3 |
| 8 | 2 | 2 |
| 9 | 2 | 3 |
| 10 | 2 | 3 |
| 11 | 2 | 3 |
| 12 | 2 | 3 |
| 13 | 2 | 1 |
| 14 | 2 | 4 |
| 15 | 2 | 3 |
| 16 | 2 | 3 |
| 17 | 2 | 3 |
| 18 | 2 | 3 |
| 19 | 2 | 3 |
| 20 | 2 | 3 |
| 21 | 2 | 3 |
| 22 | 2 | 3 |
| 23 | 2 | 3 |
| 24 | 2 | 3 |
| 25 | 2 | 3 |
| 26 | 2 | 3 |
| 27 | 2 | 3 |
| 28 | 2 | 3 |
| 29 | 2 | 3 |
| 30 | 2 | 3 |
| 31 | 2 | 3 |
| 32 | 2 | 3 |
| 33 | 2 | 3 |
| 34 | 2 | 3 |
| 35 | 2 | 3 |
| 36 | 2 | 3 |
| 37 | 2 | 3 |
| 38 | 2 | 3 |
| 39 | 2 | 3 |
| 40 | 2 | 3 |
| 41 | 2 | 3 |
| 42 | 2 | 3 |
| 43 | 2 | 1 |
| 44 | 2 | 3 |
| 45 | 2 | 3 |
| 46 | 2 | 3 |
| 47 | 2 | 3 |
| 48 | 2 | 3 |
| 49 | 2 | 3 |
| 50 | 2 | 3 |

TABLE 10d
Data for Klebsiella, particularly K. oxytoca (continued)

| No. | A/S_pv_adj | AUG_pv_adj | AZT_pv_adj | CAX_pv_adj | CFT_pv_adj |
|---|---|---|---|---|---|
| 1 | 7.25E−19 | 1.72E−03 | 1.02E−01 | 7.63E−02 | 1.14E−01 |
| 2 | 7.25E−19 | 1.72E−03 | 1.02E−01 | 7.63E−02 | 1.14E−01 |
| 3 | 9.82E−17 | 1.48E−03 | 1.97E−02 | 1.37E−02 | 6.22E−02 |
| 4 | 4.07E−16 | 1.01E−02 | 2.64E−02 | 3.46E−02 | 3.40E−02 |
| 5 | 4.07E−16 | 2.92E−04 | 3.51E−02 | 2.48E−02 | 4.69E−02 |
| 6 | 6.05E−16 | 7.34E−03 | 3.46E−02 | 2.50E−02 | 2.46E−02 |
| 7 | 6.05E−16 | 7.34E−03 | 3.46E−02 | 2.50E−02 | 2.46E−02 |
| 8 | 6.05E−16 | 9.84E−03 | 4.51E−02 | 3.38E−02 | 2.50E−02 |
| 9 | 6.05E−16 | 7.34E−03 | 3.46E−02 | 2.50E−02 | 2.46E−02 |
| 10 | 6.05E−16 | 7.34E−03 | 3.46E−02 | 2.50E−02 | 2.46E−02 |
| 11 | 6.05E−16 | 7.34E−03 | 3.46E−02 | 2.50E−02 | 2.46E−02 |
| 12 | 6.05E−16 | 7.34E−03 | 3.46E−02 | 2.50E−02 | 2.46E−02 |
| 13 | 6.05E−16 | 1.01E−02 | 4.61E−02 | 3.45E−02 | 3.46E−02 |
| 14 | 6.05E−16 | 5.13E−03 | 2.58E−02 | 1.89E−02 | 1.75E−02 |
| 15 | 6.05E−16 | 7.34E−03 | 3.46E−02 | 2.50E−02 | 2.46E−02 |
| 16 | 6.05E−16 | 7.34E−03 | 3.46E−02 | 2.50E−02 | 2.46E−02 |
| 17 | 6.05E−16 | 7.34E−03 | 3.46E−02 | 2.50E−02 | 2.46E−02 |
| 18 | 6.05E−16 | 7.34E−03 | 3.46E−02 | 2.50E−02 | 2.46E−02 |
| 19 | 6.05E−16 | 7.34E−03 | 3.46E−02 | 2.50E−02 | 2.46E−02 |
| 20 | 6.05E−16 | 7.34E−03 | 3.46E−02 | 2.50E−02 | 2.46E−02 |
| 21 | 6.05E−16 | 7.34E−03 | 3.46E−02 | 2.50E−02 | 2.46E−02 |
| 22 | 6.05E−16 | 7.34E−03 | 3.46E−02 | 2.50E−02 | 2.46E−02 |
| 23 | 6.05E−16 | 5.42E−03 | 3.46E−02 | 1.98E−02 | 2.46E−02 |
| 24 | 6.05E−16 | 7.34E−03 | 3.46E−02 | 2.50E−02 | 2.46E−02 |
| 25 | 6.05E−16 | 7.34E−03 | 3.46E−02 | 2.50E−02 | 2.46E−02 |
| 26 | 6.05E−16 | 7.34E−03 | 3.46E−02 | 2.50E−02 | 2.46E−02 |
| 27 | 6.05E−16 | 7.34E−03 | 3.46E−02 | 2.50E−02 | 2.46E−02 |
| 28 | 6.05E−16 | 7.34E−03 | 3.46E−02 | 2.50E−02 | 2.46E−02 |
| 29 | 6.05E−16 | 7.34E−03 | 3.46E−02 | 2.50E−02 | 2.46E−02 |
| 30 | 6.05E−16 | 7.34E−03 | 3.46E−02 | 2.50E−02 | 2.46E−02 |
| 31 | 6.05E−16 | 7.34E−03 | 3.46E−02 | 2.50E−02 | 2.46E−02 |
| 32 | 6.05E−16 | 7.34E−03 | 3.46E−02 | 2.50E−02 | 2.46E−02 |
| 33 | 6.05E−16 | 7.34E−03 | 3.46E−02 | 2.50E−02 | 2.46E−02 |
| 34 | 6.05E−16 | 7.34E−03 | 3.46E−02 | 2.50E−02 | 2.46E−02 |
| 35 | 6.05E−16 | 7.34E−03 | 3.46E−02 | 2.50E−02 | 2.46E−02 |
| 36 | 6.05E−16 | 7.34E−03 | 3.46E−02 | 2.50E−02 | 2.46E−02 |
| 37 | 6.05E−16 | 7.34E−03 | 3.46E−02 | 2.50E−02 | 2.46E−02 |
| 38 | 6.05E−16 | 7.42E−03 | 3.51E−02 | 2.53E−02 | 2.48E−02 |
| 39 | 6.05E−16 | 7.34E−03 | 3.46E−02 | 2.50E−02 | 2.46E−02 |
| 40 | 6.05E−16 | 7.42E−03 | 3.51E−02 | 2.53E−02 | 2.48E−02 |
| 41 | 6.05E−16 | 7.34E−03 | 3.46E−02 | 2.50E−02 | 2.46E−02 |
| 42 | 6.05E−16 | 7.42E−03 | 3.51E−02 | 2.53E−02 | 2.48E−02 |
| 43 | 6.05E−16 | 1.84E−02 | 7.73E−02 | 5.74E−02 | 4.41E−02 |
| 44 | 6.05E−16 | 7.34E−03 | 3.46E−02 | 2.50E−02 | 2.46E−02 |
| 45 | 6.05E−16 | 7.34E−03 | 3.46E−02 | 2.50E−02 | 2.46E−02 |
| 46 | 6.05E−16 | 7.34E−03 | 3.46E−02 | 2.50E−02 | 2.46E−02 |
| 47 | 6.05E−16 | 7.34E−03 | 3.46E−02 | 2.50E−02 | 2.46E−02 |
| 48 | 6.05E−16 | 7.42E−03 | 3.51E−02 | 2.53E−02 | 2.48E−02 |
| 49 | 6.05E−16 | 7.34E−03 | 3.46E−02 | 2.50E−02 | 2.46E−02 |
| 50 | 6.05E−16 | 7.34E−03 | 3.46E−02 | 2.50E−02 | 2.46E−02 |

TABLE 10e
Data for Klebsiella, particularly K. oxytoca (continued)

| No. | CP_pv_adj | CRM_pv_adj | LVX_pv_adj | P/T_pv_adj |
|---|---|---|---|---|
| 1 | 4.47E−03 | 3.22E−03 | 3.33E−05 | 4.21E−03 |
| 2 | 4.47E−03 | 3.22E−03 | 3.33E−05 | 4.21E−03 |
| 3 | 2.12E−04 | 4.16E−03 | 5.03E−07 | 3.38E−03 |
| 4 | 2.11E−03 | 1.38E−02 | 6.28E−05 | 1.93E−02 |
| 5 | 6.71E−04 | 1.62E−03 | 8.24E−07 | 2.85E−03 |
| 6 | 1.30E−03 | 7.85E−03 | 5.53E−05 | 1.37E−02 |
| 7 | 1.30E−03 | 7.85E−03 | 5.53E−05 | 1.37E−02 |
| 8 | 1.98E−03 | 1.03E−02 | 5.68E−05 | 1.89E−02 |
| 9 | 1.30E−03 | 7.85E−03 | 5.53E−05 | 1.37E−02 |
| 10 | 1.30E−03 | 7.85E−03 | 5.53E−05 | 1.37E−02 |
| 11 | 1.30E−03 | 7.85E−03 | 5.53E−05 | 1.37E−02 |
| 12 | 1.30E−03 | 7.85E−03 | 5.53E−05 | 1.37E−02 |
| 13 | 1.26E−03 | 1.04E−02 | 3.08E−05 | 1.93E−02 |
| 14 | 7.58E−04 | 6.15E−03 | 5.93E−05 | 9.93E−03 |
| 15 | 1.30E−03 | 7.85E−03 | 5.53E−05 | 1.37E−02 |
| 16 | 1.30E−03 | 7.85E−03 | 5.53E−05 | 1.37E−02 |
| 17 | 1.30E−03 | 7.85E−03 | 5.53E−05 | 1.37E−02 |
| 18 | 1.30E−03 | 7.85E−03 | 5.53E−05 | 1.37E−02 |
| 19 | 1.30E−03 | 7.85E−03 | 5.53E−05 | 1.37E−02 |
| 20 | 1.30E−03 | 7.85E−03 | 5.53E−05 | 1.37E−02 |
| 21 | 1.30E−03 | 7.85E−03 | 5.53E−05 | 1.37E−02 |
| 22 | 1.30E−03 | 7.85E−03 | 5.53E−05 | 1.37E−02 |
| 23 | 3.39E−03 | 6.15E−03 | 1.55E−04 | 1.03E−02 |
| 24 | 1.30E−03 | 7.85E−03 | 5.53E−05 | 1.37E−02 |
| 25 | 1.30E−03 | 7.85E−03 | 5.53E−05 | 1.37E−02 |
| 26 | 1.30E−03 | 7.85E−03 | 5.53E−05 | 1.37E−02 |
| 27 | 1.30E−03 | 7.85E−03 | 5.53E−05 | 1.37E−02 |
| 28 | 1.30E−03 | 7.85E−03 | 5.53E−05 | 1.37E−02 |
| 29 | 1.30E−03 | 7.85E−03 | 5.53E−05 | 1.37E−02 |
| 30 | 1.30E−03 | 7.85E−03 | 5.53E−05 | 1.37E−02 |
| 31 | 1.30E−03 | 7.85E−03 | 5.53E−05 | 1.37E−02 |
| 32 | 1.30E−03 | 7.85E−03 | 5.53E−05 | 1.37E−02 |
| 33 | 1.30E−03 | 7.85E−03 | 5.53E−05 | 1.37E−02 |
| 34 | 1.30E−03 | 7.85E−03 | 5.53E−05 | 1.37E−02 |
| 35 | 1.30E−03 | 7.85E−03 | 5.53E−05 | 1.37E−02 |
| 36 | 1.30E−03 | 7.85E−03 | 5.53E−05 | 1.37E−02 |
| 37 | 1.30E−03 | 7.85E−03 | 5.53E−05 | 1.37E−02 |
| 38 | 1.97E−03 | 7.93E−03 | 5.53E−05 | 1.39E−02 |
| 39 | 1.30E−03 | 7.85E−03 | 5.53E−05 | 1.37E−02 |
| 40 | 1.97E−03 | 7.93E−03 | 5.53E−05 | 1.39E−02 |
| 41 | 1.30E−03 | 7.85E−03 | 5.53E−05 | 1.37E−02 |
| 42 | 1.97E−03 | 7.93E−03 | 5.53E−05 | 1.39E−02 |
| 43 | 7.43E−03 | 1.01E−02 | 2.47E−04 | 3.31E−02 |
| 44 | 1.30E−03 | 7.85E−03 | 5.53E−05 | 1.37E−02 |
| 45 | 1.30E−03 | 7.85E−03 | 5.53E−05 | 1.37E−02 |
| 46 | 1.30E−03 | 7.85E−03 | 5.53E−05 | 1.37E−02 |
| 47 | 1.30E−03 | 7.85E−03 | 5.53E−05 | 1.37E−02 |
| 48 | 1.97E−03 | 4.47E−03 | 5.53E−05 | 1.39E−02 |
| 49 | 1.30E−03 | 7.85E−03 | 5.53E−05 | 1.37E−02 |
| 50 | 1.30E−03 | 7.85E−03 | 5.53E−05 | 1.37E−02 |

TABLE 10f
Data for Klebsiella, particularly K. oxytoca (continued)

| | | | | |
|---|---|---|---|---|
| No. | Locus.Tag | Gene.Symbol | GenBank.Accession | Chromosome |

| | | | |
|---|---|---|---|
| 1 | KPK_2590 | | YP_002238421 |
| 2 | HMPREF0485_01216 | | ZP_06548816 |
| 3 | EAE_11985 | | YP_004592590 |
| 4 | Kvar_2784 | | YP_003439702 |
| 5 | HMPREF0484_4675 | add | ZP_06017656 |
| 6 | Kvar_2381 | | YP_003439305 |
| 7 | KPN2242_04295 | | AEJ96779 |
| 8 | HMPREF0485_01916 | | ZP_06549516 |
| 9 | HMPREF0485_01247 | | ZP_06548847 |
| 10 | KPK_2622 | | YP_002238453 |
| 11 | Kvar_2568 | | YP_003439490 |
| 12 | Kvar_0816 | | YP_003437758 |
| 13 | Kvar_4643 | | YP_003441547 |
| 14 | Kvar_3771 | | YP_003440683 |
| 15 | Kvar_1596 | | YP_003438533 |
| 16 | HMPREF0485_03998 | | ZP_06551594 |
| 17 | Kvar_2140 | | YP_003439069 |
| 18 | Kvar_3380 | | YP_003440292 |
| 19 | HMPREF0485_00446 | | ZP_06548046 |
| 20 | KPN_00275 | | YP_001333958 |
| 21 | Kvar_1589 | | YP_003438526 |
| 22 | Kvar_1856 | | YP_003438789 |
| 23 | KP1_3016 | | YP_002919724 |
| 24 | Kvar_5099 | | YP_003442002 |
| 25 | Kvar_0479 | | YP_003437421 |
| 26 | HMPREF0485_00450 | | ZP_06548050 |
| 27 | Kvar_1586 | | YP_003438523 |
| 28 | Kvar_1598 | | YP_003438535 |
| 29 | Kvar_1597 | | YP_003438534 |
| 30 | HMPREF0485_00443 | | ZP_06548043 |
| 31 | HMPREF0485_00453 | | ZP_06548053 |
| 32 | KP1_2851 | | YP_002919569 |
| 33 | KPN2242_01120 | | AEJ96149 |
| 34 | KPK_2936 | | YP_002238762 |
| 35 | HMPREF9538_03901 | | ZP_08306208 |
| 36 | HMPREF0485_02477 | | ZP_06550077 |
| 37 | HMPREF0485_01831 | | ZP_06549431 |
| 38 | Kvar_3395 | | YP_003440307 |
| 39 | Kvar_3210 | | YP_003440127 |
| 40 | KPN_00277 | | YP_001333960 |
| 41 | Kvar_1604 | | YP_003438541 |
| 42 | Kvar_4545 | | YP_003441451 |
| 43 | HMPREF9538_05113 | | ZP_08307402 |
| 44 | Kvar_4742 | | YP_003441646 |
| 45 | Kvar_2570 | | YP_003439492 |
| 46 | Kvar_2569 | | YP_003439491 |
| 47 | KPN_04505 | melA | YP_001338127 |
| 48 | KPK_1698 | nifA | YP_002237549 |
| 49 | Dda3937_02155 | nifH | YP_003884784 |
| 50 | KPK_1712 | nifK | YP_002237563 |

TABLE 10g
Data for Klebsiella, particularly K. oxytoca (continued)

| No. | Start.Coord | End.Coord | Strand | Scaffold.ID |
|---|---|---|---|---|
| 1 | 2648871 | 2649476 | + | 643348533 |
| 2 | 1355295 | 1356167 | − | 647536389 |
| 3 | 2576463 | 2577677 | − | 650716122 |
| 4 | 2889784 | 2890686 | − | 646312028 |
| 5 | 12390 | 13076 | + | 647009977 |
| 6 | 2489613 | 2491040 | + | 646312028 |
| 7 | 925860 | 926771 | − | 651053122 |
| 8 | 237063 | 237326 | + | 647536390 |
| 9 | 1387256 | 1388314 | + | 647536389 |
| 10 | 2680912 | 2681727 | + | 643348533 |
| 11 | 2679911 | 2680687 | + | 646312028 |
| 12 | 851698 | 852411 | + | 646312028 |
| 13 | 4934923 | 4936932 | + | 646312028 |
| 14 | 3964905 | 3966425 | + | 646312028 |
| 15 | 1721712 | 1722182 | − | 646312028 |
| 16 | 140912 | 141796 | + | 647536393 |
| 17 | 2255010 | 2255996 | + | 646312028 |
| 18 | 3519306 | 3520322 | − | 646312028 |
| 19 | 523538 | 524377 | − | 647536389 |
| 20 | 311624 | 312142 | + | 640753018 |
| 21 | 1716249 | 1716779 | + | 646312028 |
| 22 | 1971206 | 1973212 | − | 646312028 |
| 23 | 2878963 | 2879583 | + | 646564530 |
| 24 | 5455245 | 5456423 | − | 646312028 |
| 25 | 509672 | 510736 | + | 646312028 |
| 26 | 528513 | 529175 | − | 647536389 |
| 27 | 1711303 | 1712709 | − | 646312028 |
| 28 | 1723564 | 1724937 | − | 646312028 |
| 29 | 1722169 | 1723554 | − | 646312028 |
| 30 | 520902 | 521159 | − | 647536389 |
| 31 | 531062 | 532510 | − | 647536389 |
| 32 | 2717504 | 2718439 | + | 646564530 |
| 33 | 230663 | 231895 | − | 651053122 |
| 34 | 2983904 | 2984626 | + | 643348533 |
| 35 | 15395 | 16450 | + | 651333420 |
| 36 | 872347 | 873972 | + | 647536390 |
| 37 | 149922 | 150884 | − | 647536390 |
| 38 | 3535340 | 3535786 | + | 646312028 |
| 39 | 3340882 | 3341376 | − | 646312028 |
| 40 | 312969 | 315452 | + | 640753018 |
| 41 | 1730820 | 1734335 | + | 646312028 |
| 42 | 4822901 | 4823620 | + | 646312028 |
| 43 | 52 | 390 | − | 651333602 |
| 44 | 5039066 | 5040004 | + | 646312028 |
| 45 | 2681431 | 2682066 | + | 646312028 |
| 46 | 2680692 | 2681420 | + | 646312028 |
| 47 | 4930190 | 4931542 | + | 640753018 |
| 48 | 1752865 | 1754439 | − | 643348533 |
| 49 | 4321869 | 4322750 | − | 648028072 |
| 50 | 1766440 | 1768002 | − | 643348533 |

TABLE 11a
Data for Klebsiella, particularly K. pneumoniae

| | Scaffold.External. | | |
|---|---|---|---|
| No. | Accession | Scaffold.Name | best_pv |
| 1 | NC_007384 | Shigella sonnei Ss046: NC_007384 | 1.21395823682916e−119 |
| 2 | NC_011083 | Salmonella enterica subsp. enterica serovar Heidelberg str. SL476: | 1.70517984237646e−115 |
| | | NC_011083 | |
| 3 | NZ_ADWV01000045 | Escherichia coli MS 107-1 E_coliMS107-1-1.0.1_Cont44.1: | 3.12333368944279e−110 |
| | | NZ_ADWV01000045 | |
| 4 | NC_009651 | Klebsiella pneumoniae subsp. pneumoniae MGH 78578 plasmid pKPN5: | 1.8490215255217e−103 |
| | | NC_009651 | |
| 5 | NZ_ABFH01000001 | Salmonella enterica subsp. enterica serovar Virchow str. SL491, | 4.16211077482132e−97 |
| | | unfinished sequence: NZ_ABFH01000001 | |
| 6 | CP002910 | Klebsiella pneumoniae KCTC 2242: CP002910 | 1.34397014479151e−80 |
| 7 | CP002910 | Klebsiella pneumoniae KCTC 2242: CP002910 | 1.21342816034335e−71 |
| 8 | NZ_ACZD01000070 | Klebsiella pneumoniae subsp. rhinoscleromatis ATCC 13884 | 4.42089689687353e−61 |
| | | contig00070: NZ_ACZD01000070 | |
| 9 | NC_013850 | Klebsiella variicola At-22 chromosome: NC_013850 | 8.41004168548543e−61 |
| 10 | NC_011282 | Klebsiella pneumoniae 342 plasmid pKP187: NC_011282 | 8.41004168548543e−61 |
| 11 | NZ_ACZD01000070 | Klebsiella pneumoniae subsp. rhinoscleromatis ATCC 13884 | 8.41004168548543e−61 |
| | | contig00070: NZ_ACZD01000070 | |
| 12 | NZ_ADTP01000323 | Escherichia coli MS 69-1 E_coli69-1-1.0_Cont630.1: NZ_ADTP01000323 | 4.94539115664213e−60 |
| 13 | NC_009793 | Citrobacter koseri ATCC BAA-895 plasmid pCK03: NC_009793 | 5.71878021049123e−59 |
| 14 | NZ_ACZD01000005 | Klebsiella pneumoniae subsp. rhinoscleromatis ATCC 13884 | 1.22977045856913e−58 |
| | | contig00005: NZ_ACZD01000005 | |
| 15 | NZ_ACZD01000127 | Klebsiella pneumoniae subsp. rhinoscleromatis ATCC 13884 | 1.49068550300511e−58 |
| | | contig00129: NZ_ACZD01000127 | |
| 16 | NZ_GG745515 | Klebsiella sp. 1_1_55 genomic scaffold supercont1.8: NZ_GG745515 | 7.09566707309011e−58 |
| 17 | NZ_AAJX01000089 | Escherichia coli B171, unfinished sequence: NZ_AAJX01000089 | 1.65409147045657e−56 |
| 18 | NZ_ACZD01000005 | Klebsiella pneumoniae subsp. rhinoscleromatis ATCC 13884 | 7.54090407593199e−55 |
| | | contig00005: NZ_ACZD01000005 | |
| 19 | NZ_AFBO01000042 | Klebsiella sp. MS 92-3 K_spMS92-3-1.0_Cont91.1: NZ_AFBO01000042 | 2.8348333685311e−54 |
| 20 | NC_009649 | Klebsiella pneumoniae subsp. pneumoniae MGH 78578 plasmid pKPN3: | 2.8348333685311e−54 |
| | | NC_009649 | |
| 21 | CP002910 | Klebsiella pneumoniae KCTC 2242: CP002910 | 2.83530891190408e−54 |
| 22 | NC_009649 | Klebsiella pneumoniae subsp. pneumoniae MGH 78578 plasmid pKPN3: | 4.89541334527925e−54 |
| | | NC_009649 | |
| 23 | NZ_AFBO01000042 | Klebsiella sp. MS 92-3 K_spMS92-3-1.0_Cont91.1: NZ_AFBO01000042 | 2.18592863711471e−53 |
| 24 | NC_009649 | Klebsiella pneumoniae subsp. pneumoniae MGH 78578 plasmid pKPN3: | 2.18592863711471e−53 |
| | | NC_009649 | |
| 25 | NZ_ACZD01000127 | Klebsiella pneumoniae subsp. rhinoscleromatis ATCC 13884 | 1.08576020331202e−52 |
| | | contig00129: NZ_ACZD01000127 | |
| 26 | NZ_ACZD01000127 | Klebsiella pneumoniae subsp. rhinoscleromatis ATCC 13884 | 1.37521017661703e−52 |
| | | contig00129: NZ_ACZD01000127 | |
| 27 | NZ_ACZD01000127 | Klebsiella pneumoniae subsp. rhinoscleromatis ATCC 13884 | 2.1362800317976e−52 |
| | | contig00129: NZ_ACZD01000127 | |
| 28 | NC_014121 | Enterobacter cloacae subsp. cloacae ATCC 13047 chromosome: NC_014121 | 2.21381524453565e−51 |
| 29 | CP002910 | Klebsiella pneumoniae KCTC 2242: CP002910 | 6.53846908249994e−51 |
| 30 | NZ_GG745512 | Klebsiella sp. 1_1_55 genomic scaffold supercont1.5: NZ_GG745512 | 2.78992804201171e−50 |
| 31 | NZ_CAAZ01000154 | Salmonella enterica subsp. enterica serovar Typhi str. E98-3139, | 4.10919139010377e−50 |
| | | unfinished sequence: NZ_CAAZ01000154 | |
| 32 | NZ_ACZD01000127 | Klebsiella pneumoniae subsp. rhinoscleromatis ATCC 13884 | 6.10670380866266e−50 |
| | | contig00129: NZ_ACZD01000127 | |
| 33 | NZ_ACZD01000127 | Klebsiella pneumoniae subsp. rhinoscleromatis ATCC 13884 | 1.00996840886331e−49 |
| | | contig00129: NZ_ACZD01000127 | |
| 34 | CP002910 | Klebsiella pneumoniae KCTC 2242: CP002910 | 1.33344542353295e−48 |
| 35 | CP002910 | Klebsiella pneumoniae KCTC 2242: CP002910 | 3.00269800428115e−48 |
| 36 | CP002910 | Klebsiella pneumoniae KCTC 2242: CP002910 | 3.13126971411145e−48 |
| 37 | NZ_ACZD01000127 | Klebsiella pneumoniae subsp. rhinoscleromatis ATCC 13884 | 3.13126971411145e−48 |
| | | contig00129: NZ_ACZD01000127 | |
| 38 | NC_014121 | Enterobacter cloacae subsp. cloacae ATCC 13047 chromosome: NC_014121 | 3.79149056816214e−48 |
| 39 | NZ_AFBO01000625 | Klebsiella sp. MS 92-3 K_spMS92-3-1.0_Cont1175.2: NZ_AFBO01000625 | 2.5975548233422e−47 |
| 40 | NZ_ACZD01000127 | Klebsiella pneumoniae subsp. rhinoscleromatis ATCC 13884 | 1.05391699509183e−46 |
| | | contig00129: NZ_ACZD01000127 | |
| 41 | NC_011092 | Salmonella enterica subsp. enterica serovar Schwarzengrund str. | 4.42178242563847e−46 |
| | | CVM19633 plasmid pCVM19633_110: NC_011092 | |
| 42 | NC_013850 | Klebsiella variicola At-22 chromosome: NC_013850 | 6.65483065085068e−46 |
| 43 | NC_012912 | Dickeya zeae Ech1591: NC_012912 | 1.05262166415392e−45 |
| 44 | NC_013509 | Edwardsiella tarda EIB202 plasmid pEIB202: NC_013509 | 1.24074600175825e−45 |
| 45 | AP011957 | Salmonella enterica subsp. enterica serovar Typhimurium str. T000240 | 2.46270503642371e−45 |
| | | DNA: AP011957 | |
| 46 | NC_006856 | Salmonella enterica subsp. enterica serovar Choleraesuis str. SC-B67 | 9.02631418377704e−45 |
| | | plasmid pSC138: NC_006856 | |
| 47 | AP011957 | Salmonella enterica subsp. enterica serovar Typhimurium str. T000240 | 3.37821430101762e−44 |
| | | DNA: AP011957 | |
| 48 | NZ_AFBO01000232 | Klebsiella sp. MS 92-3 K_spMS92-3-1.0_Cont505.1: NZ_AFBO01000232 | 4.87973844618098e−43 |
| 49 | NC_004631 | Salmonella enterica subsp. enterica serovar Typhi Ty2: NC_004631 | 1.54269244019591e−42 |
| 50 | NC_006625 | Klebsiella pneumoniae NTUH-K2044 plasmid pK2044: NC_006625 | 1.89874529968327e−42 |

TABLE 11b
Data for Klebsiella, particularly K. pneumoniae (continued)

| No. | sign_phenos | sign_phenos_class | best_pheno | best_pheno_class |
|---|---|---|---|---|
| 1 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 2 | A/S; AUG; AZT; CAX; CAZ; CFT; CP; CPE; CRM; | aminoglycoside; fluoroquinolone; lactam; other | AUG | lactam |
| | ETP; GM; LVX; P/T; TO; T/S | | | |
| 3 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 4 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 5 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 6 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 7 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 8 | A/S; AUG; AZT; CAX; CAZ; CFT; CP; CPE; CRM; | aminoglycoside; fluoroquinolone; lactam; other | ETP | lactam |
| | ETP; CM; LVX; P/T; TO; T/S | | | |
| 9 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 10 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 11 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 12 | A/S; AUG; AZT; CAX; CAZ; CFT; CP; CPE; CRM; | aminoglycoside; fluoroquinolone; lactam; other | CAZ | lactam |
| | ETP; GM; LVX; P/T; TO; T/S | | | |
| 13 | A/S; AUG; AZT; CAX; CAZ; CFT; CP; CPE; CRM; | aminoglycoside; fluoroquinolone; lactam; other | AUG | lactam |
| | ETP; GM; LVX; P/T; TO; T/S | | | |
| 14 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 15 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 16 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 17 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 18 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 19 | A/S; AUG; AZT; CAX; CAZ; CFT; CP; CPE; CRM; | aminoglycoside; fluoroquinolone; lactam; other | P/T | lactam |
| | ETP; GM; LVX; P/T; TO; T/S | | | |
| 20 | A/S; AUG; AZT; CAX; CAZ; CFT; CP; CPE; CRM; | aminoglycoside; fluoroquinolone; lactam; other | P/T | lactam |
| | ETP; GM; LVX; P/T; TO; T/S | | | |
| 21 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 22 | A/S; AUG; AZT; CAX; CAZ; CFT; CP; CPE; CRM; | aminoglycoside; fluoroquinolone; lactam; other | P/T | lactam |
| | ETP; GM; LVX; P/T; TO; T/S | | | |
| 23 | A/S; AUG; AZT; CAX; CAZ; CFT; CP; CPE; CRM; | aminoglycoside; fluoroquinolone; lactam; other | P/T | lactam |
| | ETP; GM; LVX; P/T; TO; T/S | | | |
| 24 | A/S; AUG; AZT; CAX; CAZ; CFT; CP; CPE; CRM; | aminoglycoside; fluoroquinolone; lactam; other | P/T | lactam |
| | ETP; GM; LVX; P/T; TO; T/S | | | |
| 25 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 26 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 27 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 28 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 29 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 30 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 31 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 32 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 33 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 34 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 35 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 36 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 37 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 38 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 39 | A/S; AUG; AZT; CAX; CAZ; CFT; CP; CPE; CRM; | aminoglycoside; fluoroquinolone; lactam; other | T/S | other |
| | CM; LVX; P/T; TO; T/S | | | |
| 40 | A/S; AUG; AZT; CAX; CAZ; CFT; CP; CPE; CRM; | aminoglycoside; fluoroquinolone; lactam; other | CP | fluoroquinolone |
| | ETP; GM; LVX; P/T; TO; T/S | | | |
| 41 | A/S; AUG; AZT; CAX; CAZ; CFT; CP; CPE; CRM; | aminoglycoside; fluoroquinolone; lactam; other | AUG | lactam |
| | ETP; GM; LVX; P/T; TO; T/S | | | |
| 42 | AUG; AZT; CAX; CAZ; CFT; CP; CPE; CRM; ETP ; | aminoglycoside; fluoroquinolone; lactam; other | ETP | lactam |
| | LVX; P/T; TO; T/S | | | |
| 43 | AUG; AZT; CAX; CAZ; CFT; CP; CPE; CRM; ETP ; | aminoglycoside; fluoroquinolone; lactam; other | ETP | lactam |
| | LVX; P/T; TO; T/S | | | |
| 44 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 45 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 46 | A/S; AUG; AZT; CAX; CAZ; CFT; CP; CPE; CRM; | aminoglycoside; fluoroquinolone; lactam; other | TO | aminoglycoside |
| | ETP; GM; LVX; P/T; TO; T/S | | | |
| 47 | A/S; AUG; AZT; CAX; CAZ; CFT; CP; CPE; CRM; | aminoglycoside; fluoroquinolone; lactam; other | AUG | lactam |
| | ETP; GM; LVX; P/T; TO; T/S | | | |
| 48 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |
| 49 | A/S; AUG; AZT; CAX; CAZ; CFT; CP; CPE; CRM; | aminoglycoside; fluoroquinolone; lactam; other | ETP | lactam |
| | ETP; GM; LVX; P/T; TO; T/S | | | |
| 50 | A/S; AUG; AZT; CAX; CAZ; CFT; CP; CPE; CRM; | aminoglycoside; fluoroquinolone; lactam; other | T/S | other |
| | ETP; GM; LVX; P/T; TO; T/S | | | |

TABLE 11c
Data for Klebsiella, particularly K. pneumoniae (continued)

| No. | num_aminoglycoside | num_fluoroquinolone | num_lactam | num_other |
|---|---|---|---|---|
| 1 | 2 | 2 | 10 | 1 |
| 2 | 2 | 2 | 10 | 1 |
| 3 | 2 | 2 | 10 | 1 |
| 4 | 2 | 2 | 10 | 1 |
| 5 | 2 | 2 | 10 | 1 |
| 6 | 2 | 2 | 10 | 1 |
| 7 | 2 | 2 | 10 | 1 |
| 8 | 2 | 2 | 10 | 1 |
| 9 | 2 | 2 | 10 | 1 |
| 10 | 2 | 2 | 10 | 1 |
| 11 | 2 | 2 | 10 | 1 |
| 12 | 2 | 2 | 10 | 1 |
| 13 | 2 | 2 | 10 | 1 |
| 14 | 2 | 2 | 10 | 1 |
| 15 | 2 | 2 | 10 | 1 |
| 16 | 2 | 2 | 10 | 1 |
| 17 | 2 | 2 | 10 | 1 |
| 18 | 2 | 2 | 10 | 1 |
| 19 | 2 | 2 | 10 | 1 |
| 20 | 2 | 2 | 10 | 1 |
| 21 | 2 | 2 | 10 | 1 |
| 22 | 2 | 2 | 10 | 1 |
| 23 | 2 | 2 | 10 | 1 |
| 24 | 2 | 2 | 10 | 1 |
| 25 | 2 | 2 | 10 | 1 |
| 26 | 2 | 2 | 10 | 1 |
| 27 | 2 | 2 | 10 | 1 |
| 28 | 2 | 2 | 10 | 1 |
| 29 | 2 | 2 | 10 | 1 |
| 30 | 2 | 2 | 10 | 1 |
| 31 | 2 | 2 | 10 | 1 |
| 32 | 2 | 2 | 10 | 1 |
| 33 | 2 | 2 | 10 | 1 |
| 34 | 2 | 2 | 10 | 1 |
| 35 | 2 | 2 | 10 | 1 |
| 36 | 2 | 2 | 10 | 1 |
| 37 | 2 | 2 | 10 | 1 |
| 38 | 2 | 2 | 10 | 1 |
| 39 | 2 | 2 | 9 | 1 |
| 40 | 2 | 2 | 10 | 1 |
| 41 | 2 | 2 | 10 | 1 |
| 42 | 1 | 2 | 9 | 1 |
| 43 | 1 | 2 | 9 | 1 |
| 44 | 2 | 2 | 10 | 1 |
| 45 | 2 | 2 | 10 | 1 |
| 46 | 2 | 2 | 10 | 1 |
| 47 | 2 | 2 | 10 | 1 |
| 48 | 2 | 2 | 10 | 1 |
| 49 | 2 | 2 | 10 | 1 |
| 50 | 2 | 2 | 10 | 1 |

TABLE 11d
Data for Klebsiella, particularly K. pneumoniae (continued)

| No. | A/S_pv_adj | AUG_pv_adj | AZT_pv_adj | CAX_pv_adj | CAZ_pv_adj |
|---|---|---|---|---|---|
| 1 | 1.28E−76 | 1.24E−105 | 4.06E−95 | 3.39E−95 | 5.99E−93 |
| 2 | 1.18E−98 | 1.71E−115 | 9.09E−91 | 6.55E−88 | 1.66E−96 |
| 3 | 1.08E−81 | 5.85E−86 | 2.76E−94 | 4.16E−97 | 6.84E−100 |
| 4 | 5.55E−76 | 3.05E−84 | 1.38E−91 | 2.64E−96 | 5.33E−94 |
| 5 | 1.22E−70 | 3.41E−71 | 1.41E−80 | 1.50E−83 | 6.22E−90 |
| 6 | 7.70E−21 | 1.03E−41 | 8.20E−48 | 2.50E−44 | 2.69E−46 |
| 7 | 8.54E−16 | 4.34E−29 | 4.54E−38 | 5.50E−34 | 1.34E−34 |
| 8 | 2.62E−12 | 1.74E−24 | 1.49E−26 | 2.74E−25 | 2.84E−24 |
| 9 | 5.20E−12 | 2.50E−24 | 3.69E−26 | 7.31E−25 | 7.66E−24 |
| 10 | 5.20E−12 | 2.50E−24 | 3.69E−26 | 7.31E−25 | 7.66E−24 |
| 11 | 5.20E−12 | 2.50E−24 | 3.69E−26 | 7.31E−25 | 7.66E−24 |
| 12 | 3.32E−43 | 1.75E−59 | 1.48E−54 | 9.28E−55 | 4.95E−60 |
| 13 | 2.41E−43 | 5.72E−59 | 4.20E−53 | 2.96E−53 | 2.14E−58 |
| 14 | 2.47E−16 | 8.57E−27 | 1.49E−34 | 1.96E−31 | 8.83E−33 |
| 15 | 1.72E−35 | 6.40E−55 | 1.25E−44 | 4.12E−43 | 1.61E−46 |
| 16 | 3.04E−18 | 5.19E−32 | 4.83E−36 | 2.23E−35 | 1.91E−32 |
| 17 | 1.02E−47 | 9.19E−45 | 1.85E−48 | 9.17E−53 | 6.18E−56 |
| 18 | 3.51E−17 | 3.04E−23 | 8.29E−30 | 1.21E−27 | 9.29E−29 |
| 19 | 1.03E−40 | 7.90E−52 | 1.41E−37 | 4.81E−39 | 9.97E−38 |
| 20 | 1.03E−40 | 7.90E−52 | 1.41E−37 | 4.81E−39 | 9.97E−38 |
| 21 | 8.56E−34 | 4.84E−53 | 8.75E−43 | 2.41E−41 | 9.43E−45 |
| 22 | 5.13E−42 | 1.10E−52 | 3.02E−38 | 9.55E−40 | 3.15E−38 |
| 23 | 1.11E−41 | 3.84E−52 | 1.00E−37 | 3.11E−39 | 8.03E−38 |
| 24 | 1.11E−41 | 3.84E−52 | 1.00E−37 | 3.11E−39 | 8.03E−38 |
| 25 | 8.85E−33 | 1.54E−51 | 2.83E−42 | 1.24E−40 | 2.88E−44 |
| 26 | 1.96E−32 | 2.54E−52 | 2.98E−42 | 1.41E−40 | 3.55E−44 |
| 27 | 5.12E−32 | 6.70E−50 | 1.84E−40 | 2.34E−38 | 1.33E−42 |
| 28 | 1.65E−30 | 3.00E−47 | 1.96E−38 | 1.37E−36 | 4.94E−39 |
| 29 | 4.43E−31 | 1.45E−47 | 2.29E−39 | 1.06E−37 | 3.63E−40 |
| 30 | 6.99E−12 | 2.37E−26 | 2.77E−24 | 5.28E−24 | 2.55E−26 |
| 31 | 2.78E−31 | 7.62E−46 | 1.11E−38 | 5.40E−37 | 1.95E−39 |
| 32 | 9.62E−32 | 1.45E−47 | 2.29E−39 | 1.06E−37 | 3.63E−40 |
| 33 | 2.27E−31 | 2.28E−47 | 3.20E−39 | 1.66E−37 | 5.77E−40 |
| 34 | 9.74E−28 | 9.40E−43 | 1.44E−35 | 7.30E−34 | 3.24E−36 |
| 35 | 1.57E−28 | 2.77E−42 | 6.21E−36 | 1.59E−34 | 2.14E−36 |
| 36 | 3.41E−27 | 2.34E−43 | 7.21E−36 | 4.53E−33 | 9.07E−37 |
| 37 | 3.41E−27 | 2.34E−43 | 7.21E−36 | 4.53E−33 | 9.07E−37 |
| 38 | 2.00E−30 | 7.61E−47 | 1.00E−38 | 1.06E−37 | 3.63E−40 |
| 39 | 8.29E−47 | 2.48E−33 | 3.67E−37 | 5.57E−42 | 2.74E−43 |
| 40 | 1.53E−27 | 6.97E−43 | 2.91E−36 | 2.70E−35 | 5.96E−38 |
| 41 | 1.75E−44 | 4.42E−46 | 2.16E−37 | 1.21E−36 | 1.12E−42 |
| 42 | 4.51E−02 | 4.66E−12 | 9.77E−17 | 3.13E−16 | 9.90E−15 |
| 43 | 5.37E−02 | 5.73E−12 | 1.27E−16 | 6.11E−16 | 1.15E−14 |
| 44 | 6.65E−31 | 4.17E−32 | 1.40E−25 | 3.69E−27 | 1.78E−29 |
| 45 | 6.15E−34 | 5.78E−41 | 2.29E−36 | 9.94E−38 | 1.48E−38 |
| 46 | 3.01E−32 | 3.12E−38 | 7.22E−37 | 3.96E−36 | 6.00E−37 |
| 47 | 4.35E−41 | 3.38E−44 | 1.49E−30 | 1.42E−33 | 1.82E−29 |
| 48 | 6.41E−31 | 1.63E−41 | 3.24E−33 | 2.26E−36 | 1.97E−31 |
| 49 | 2.54E−11 | 2.61E−18 | 8.71E−21 | 1.23E−18 | 1.87E−19 |
| 50 | 2.38E−23 | 3.76E−29 | 2.21E−27 | 4.97E−29 | 1.37E−29 |

TABLE 11e
Data for Klebsiella, particularly K. pneumoniae (continued)

| No. | CFT_pv_adj | CP_pv_adj | CPE_pv_adj | CRM_pv_adj | ETP_pv_adj |
|---|---|---|---|---|---|
| 1 | 2.17E−99 | 2.40E−85 | 2.31E−68 | 1.21E−82 | 1.87E−58 |
| 2 | 2.52E−83 | 1.28E−61 | 4.61E−66 | 7.83E−57 | 8.20E−51 |
| 3 | 3.93E−93 | 2.17E−58 | 2.89E−43 | 3.32E−73 | 3.54E−24 |
| 4 | 1.52E−90 | 4.25E−60 | 1.75E−43 | 2.59E−71 | 1.63E−23 |
| 5 | 3.61E−77 | 1.76E−47 | 1.47E−36 | 5.14E−62 | 6.31E−21 |
| 6 | 2.84E−46 | 1.53E−54 | 2.89E−63 | 1.06E−36 | 1.34E−80 |
| 7 | 8.43E−36 | 1.96E−43 | 3.06E−55 | 3.95E−29 | 1.21E−71 |
| 8 | 5.55E−27 | 2.63E−33 | 6.90E−40 | 1.01E−20 | 4.42E−61 |
| 9 | 1.58E−26 | 5.49E−33 | 1.32E−39 | 2.40E−20 | 8.41E−61 |
| 10 | 1.58E−26 | 5.49E−33 | 1.32E−39 | 2.40E−20 | 8.41E−61 |
| 11 | 1.58E−26 | 5.49E−33 | 1.32E−39 | 4.98E−21 | 8.41E−61 |
| 12 | 4.39E−54 | 4.96E−60 | 1.80E−51 | 7.75E−50 | 7.02E−53 |
| 13 | 1.26E−52 | 1.35E−58 | 1.72E−50 | 4.57E−49 | 3.71E−52 |
| 14 | 1.28E−31 | 2.00E−30 | 6.27E−42 | 1.20E−29 | 1.23E−58 |
| 15 | 2.05E−41 | 2.56E−52 | 7.76E−46 | 3.74E−32 | 5.20E−48 |
| 16 | 1.52E−36 | 2.10E−37 | 5.58E−48 | 5.62E−28 | 7.10E−58 |
| 17 | 9.26E−49 | 4.16E−22 | 1.22E−19 | 9.42E−36 | 5.55E−05 |
| 18 | 6.27E−28 | 1.02E−25 | 4.83E−36 | 7.62E−29 | 7.54E−55 |
| 19 | 9.92E−40 | 2.65E−39 | 2.28E−41 | 4.71E−28 | 1.10E−32 |
| 20 | 9.92E−40 | 2.65E−39 | 2.28E−41 | 4.71E−28 | 1.10E−32 |
| 21 | 1.47E−40 | 1.18E−49 | 1.74E−45 | 2.18E−32 | 1.46E−46 |
| 22 | 2.00E−40 | 6.22E−40 | 7.04E−42 | 1.08E−28 | 5.10E−33 |
| 23 | 6.77E−40 | 2.32E−39 | 3.78E−41 | 2.22E−28 | 3.70E−32 |
| 24 | 6.77E−40 | 2.32E−39 | 3.78E−41 | 2.22E−28 | 3.70E−32 |
| 25 | 4.69E−40 | 3.62E−49 | 1.06E−44 | 1.70E−33 | 2.11E−45 |
| 26 | 4.89E−40 | 1.97E−49 | 1.88E−45 | 2.34E−32 | 8.59E−46 |
| 27 | 5.94E−39 | 1.13E−47 | 2.46E−43 | 3.44E−33 | 1.93E−49 |
| 28 | 3.32E−36 | 2.21E−51 | 1.87E−44 | 1.56E−31 | 1.83E−41 |
| 29 | 2.69E−37 | 6.54E−51 | 4.48E−44 | 2.19E−30 | 3.44E−41 |
| 30 | 1.39E−24 | 6.75E−31 | 1.12E−35 | 5.11E−15 | 2.79E−50 |
| 31 | 1.34E−36 | 4.11E−50 | 1.67E−43 | 1.45E−31 | 8.85E−41 |
| 32 | 2.69E−37 | 6.11E−50 | 4.48E−44 | 2.19E−30 | 3.44E−41 |
| 33 | 6.74E−37 | 1.01E−49 | 6.91E−44 | 1.07E−30 | 4.69E−41 |
| 34 | 3.28E−34 | 1.33E−48 | 1.41E−40 | 1.52E−30 | 7.40E−43 |
| 35 | 3.72E−36 | 3.00E−48 | 1.67E−40 | 8.05E−31 | 2.56E−43 |
| 36 | 1.04E−34 | 3.13E−48 | 5.88E−41 | 1.06E−29 | 3.84E−43 |
| 37 | 1.04E−34 | 3.13E−48 | 5.88E−41 | 1.06E−29 | 3.84E−43 |
| 38 | 2.69E−37 | 3.79E−48 | 4.21E−43 | 9.89E−32 | 5.95E−40 |
| 39 | 2.84E−38 | 3.78E−14 | 1.15E−05 | 9.70E−31 | 9.25E−01 |
| 40 | 1.88E−36 | 1.05E−46 | 3.80E−41 | 1.01E−30 | 2.77E−43 |
| 41 | 4.01E−36 | 1.78E−19 | 2.93E−16 | 1.27E−21 | 2.46E−10 |
| 42 | 5.45E−16 | 4.08E−18 | 6.25E−28 | 7.56E−08 | 6.65E−46 |
| 43 | 1.13E−15 | 5.57E−18 | 9.82E−28 | 1.16E−07 | 1.05E−45 |
| 44 | 2.55E−25 | 6.58E−14 | 7.47E−22 | 3.89E−16 | 2.18E−20 |
| 45 | 5.14E−35 | 2.41E−27 | 5.02E−15 | 4.61E−29 | 2.22E−12 |
| 46 | 1.40E−36 | 2.78E−33 | 1.06E−29 | 5.12E−30 | 2.25E−28 |
| 47 | 3.46E−32 | 1.18E−32 | 1.62E−32 | 2.49E−29 | 1.62E−26 |
| 48 | 6.74E−36 | 1.36E−34 | 2.38E−34 | 1.27E−25 | 6.79E−29 |
| 49 | 9.64E−20 | 3.40E−22 | 1.36E−30 | 8.87E−18 | 1.54E−42 |
| 50 | 1.19E−26 | 1.97E−18 | 9.42E−19 | 1.46E−19 | 1.52E−12 |

TABLE 11f
Data for Klebsiella, particularly K. pneumoniae (continued)

| No. | GM_pv_adj | LVX_pv_adj | P/T_pv_adj | TO_pv_adj | T/S_pv_adj |
|---|---|---|---|---|---|
| 1 | 6.24E−60 | 3.97E−80 | 7.18E−96 | 1.21E−19 | 1.12E−106 |
| 2 | 6.10E−67 | 3.76E−55 | 1.20E−67 | 2.77E−05 | 3.59E−83 |
| 3 | 6.00E−68 | 3.14E−50 | 5.58E−70 | 1.37E−91 | 3.12E−110 |
| 4 | 3.06E−63 | 4.39E−52 | 7.17E−70 | 6.65E−91 | 1.85E−103 |
| 5 | 5.29E−59 | 6.80E−39 | 4.00E−51 | 1.40E−82 | 4.16E−97 |
| 6 | 5.64E−09 | 3.19E−59 | 3.01E−52 | 7.11E−48 | 1.50E−31 |
| 7 | 1.61E−05 | 6.12E−48 | 2.20E−40 | 1.45E−34 | 2.10E−20 |
| 8 | 1.57E−06 | 6.85E−37 | 6.06E−35 | 1.79E−29 | 2.02E−18 |
| 9 | 2.46E−06 | 1.41E−36 | 1.30E−34 | 3.62E−29 | 2.62E−18 |
| 10 | 2.46E−06 | 1.41E−36 | 1.30E−34 | 3.62E−29 | 2.62E−18 |
| 11 | 2.46E−06 | 1.41E−36 | 1.30E−34 | 3.62E−29 | 2.62E−18 |
| 12 | 2.30E−25 | 1.32E−53 | 6.01E−52 | 5.28E−59 | 5.07E−39 |
| 13 | 1.61E−25 | 2.14E−52 | 1.19E−50 | 1.22E−58 | 6.30E−38 |
| 14 | 6.89E−05 | 6.38E−32 | 9.66E−29 | 7.19E−31 | 3.05E−15 |
| 15 | 1.55E−23 | 1.89E−48 | 1.36E−48 | 1.49E−58 | 1.23E−46 |
| 16 | 7.14E−10 | 1.99E−38 | 1.30E−34 | 9.13E−37 | 1.47E−21 |
| 17 | 6.27E−40 | 9.96E−18 | 4.98E−31 | 1.50E−43 | 1.65E−56 |
| 18 | 7.35E−05 | 4.57E−28 | 1.61E−25 | 2.68E−27 | 2.50E−15 |
| 19 | 1.16E−19 | 5.28E−36 | 2.83E−54 | 2.59E−42 | 9.74E−48 |
| 20 | 1.16E−19 | 5.28E−36 | 2.83E−54 | 2.59E−42 | 9.74E−48 |
| 21 | 6.44E−22 | 5.93E−45 | 3.28E−47 | 2.84E−54 | 2.84E−46 |
| 22 | 6.19E−20 | 1.49E−36 | 4.90E−54 | 5.19E−43 | 1.61E−47 |
| 23 | 4.80E−20 | 5.67E−36 | 2.19E−53 | 1.90E−42 | 7.35E−49 |
| 24 | 1.78E−19 | 5.67E−36 | 2.19E−53 | 1.90E−42 | 6.83E−48 |
| 25 | 1.71E−21 | 3.92E−44 | 2.20E−46 | 1.09E−52 | 8.26E−45 |
| 26 | 1.49E−20 | 1.39E−44 | 1.02E−46 | 1.38E−52 | 9.52E−46 |
| 27 | 9.23E−19 | 4.68E−44 | 1.13E−48 | 2.14E−52 | 7.18E−42 |
| 28 | 1.04E−18 | 2.43E−47 | 2.68E−42 | 8.73E−48 | 1.22E−43 |
| 29 | 1.81E−19 | 6.48E−47 | 5.36E−42 | 7.69E−49 | 5.28E−43 |
| 30 | 1.67E−04 | 1.19E−33 | 1.48E−27 | 1.33E−24 | 2.01E−19 |
| 31 | 2.71E−19 | 3.53E−46 | 2.81E−41 | 2.57E−47 | 3.11E−43 |
| 32 | 3.53E−20 | 6.26E−46 | 5.36E−42 | 7.69E−49 | 5.57E−44 |
| 33 | 4.08E−20 | 9.51E−46 | 1.44E−41 | 1.11E−48 | 8.48E−44 |
| 34 | 9.00E−16 | 3.30E−45 | 1.53E−41 | 3.99E−46 | 1.50E−39 |
| 35 | 4.69E−14 | 4.82E−45 | 1.45E−39 | 3.68E−47 | 5.85E−39 |
| 36 | 6.72E−15 | 8.68E−45 | 4.34E−42 | 8.00E−45 | 1.33E−38 |
| 37 | 6.72E−15 | 8.68E−45 | 4.34E−42 | 8.00E−45 | 1.33E−38 |
| 38 | 4.79E−19 | 4.19E−45 | 5.36E−42 | 3.42E−47 | 5.28E−43 |
| 39 | 1.94E−40 | 1.06E−10 | 6.27E−21 | 4.23E−35 | 2.60E−47 |
| 40 | 1.53E−15 | 3.13E−43 | 2.72E−42 | 3.02E−45 | 1.22E−39 |
| 41 | 9.94E−38 | 3.55E−16 | 4.46E−22 | 7.61E−44 | 1.24E−41 |
| 42 | 2.34E−02 | 4.47E−21 | 3.44E−16 | 3.10E−15 | 2.24E−09 |
| 43 | 2.39E−02 | 1.02E−20 | 4.32E−16 | 6.38E−15 | 3.86E−09 |
| 44 | 3.44E−22 | 5.51E−13 | 6.56E−18 | 4.39E−24 | 1.24E−45 |
| 45 | 1.36E−21 | 1.14E−24 | 2.38E−33 | 2.46E−45 | 1.53E−29 |
| 46 | 1.36E−19 | 4.39E−32 | 1.03E−35 | 9.03E−45 | 5.01E−44 |
| 47 | 1.18E−14 | 5.29E−30 | 3.76E−40 | 3.55E−44 | 5.56E−38 |
| 48 | 4.91E−17 | 8.23E−33 | 1.06E−40 | 6.11E−39 | 4.88E−43 |
| 49 | 1.35E−04 | 2.25E−22 | 1.78E−19 | 5.28E−22 | 2.63E−12 |
| 50 | 8.68E−26 | 1.45E−15 | 1.73E−22 | 6.68E−34 | 1.90E−42 |

TABLE 11g
Data for Klebsiella, particularly K. pneumoniae (continued)

| No. | Locus.Tag | Gene.Symbol | GenBank.Accession | Chromosome |
|---|---|---|---|---|
| 1 | SSON_3896 | aadA | YP_312670 | |
| 2 | SeHA_C1580 | blaT | YP_002045448 | |
| 3 | HMPREF9345_05066 | | ZP_07100147 | |
| 4 | KPN_pKPN5p08201 | | YP_001338811 | plasmid pKPN5 |
| 5 | Salmoentericaenterica_010100000245 | | ZP_02702077 | |
| 6 | KPN2242_07235 | | AEJ97365 | |
| 7 | KPN2242_00750 | | AEJ96075 | |
| 8 | HMPREF0484_1390 | mgtA | ZP_06014374 | |
| 9 | Kvar_3592 | | YP_003440504 | |
| 10 | KPK_A0103 | | YP_002235751 | plasmid pKP187 |
| 11 | HMPREF0484_1392 | pacL | ZP_06014376 | |
| 12 | HMPREF9534_04427 | | ZP_07188952 | |
| 13 | CKO_pCKO3p06155 | | YP_001456633 | plasmid pCKO3 |
| 14 | HMPREF0484_0044 | vmtV | ZP_06013029 | |
| 15 | HMPREF0484_2697 | | ZP_06015679 | |
| 16 | HMPREF0485_04762 | | ZP_06552358 | |
| 17 | EcolB_01004576 | | ZP_00708527 | |
| 18 | HMPREF0484_0046 | | ZP_06013031 | |
| 19 | HMPREF9538_00440 | | ZP_08302804 | |
| 20 | KPN_pKPN3p05893 | ptxE | YP_001338502 | plasmid pKPN3 |
| 21 | KPN2242_07425 | | AEJ97403 | |
| 22 | KPN_pKPN3p05890 | ptxB | YP_001338499 | plasmid pKPN3 |
| 23 | HMPREF9538_00443 | | ZP_08302807 | |
| 24 | KPN_pKPN3p05891 | ptxC | YP_001338500 | plasmid pKPN3 |
| 25 | HMPREF0484_2686 | | ZP_06015668 | |
| 26 | HMPREF0484_2687 | gpw | ZP_06015669 | |
| 27 | HMPREF0484_2684 | | ZP_06015666 | |
| 28 | ECL_04426 | | YP_003614904 | |
| 29 | KPN2242_07415 | | AEJ97401 | |
| 30 | HMPREF0485_03923 | | ZP_06551519 | |
| 31 | Salmonellentericaenterica_010100016432 | | ZP_03412648 | |
| 32 | HMPREF0484_2698 | | ZP_06015680 | |
| 33 | HMPREF0484_2694 | fii | ZP_06015676 | |
| 34 | KPN2242_07345 | | AEJ97387 | |
| 35 | KPN2242_07330 | | AEJ97384 | |
| 36 | KPN2242_07325 | | AEJ97383 | |
| 37 | HMPREF0484_2676 | | ZP_06015658 | |
| 38 | ECL_04434 | | YP_003614912 | |
| 39 | HMPREF9538_04737 | | ZP_08307036 | |
| 40 | HMPREF0484_2673 | | ZP_06015655 | |
| 41 | SeSA_B0079 | | YP_002112958 | plasmid |
| | | | | pCVM19633_110 |
| 42 | Kvar_2527 | | YP_003439449 | |
| 43 | Dd1591_2579 | | YP_003004893 | |
| 44 | ETAE_p041 | folP | YP_003297635 | plasmid pEIB202 |
| 45 | STMDT12_C39340 | | BAJ38877 | |
| 46 | SC026 | aph | YP_209331 | plasmid pSC138 |
| 47 | STMDT12_C39170 | | BAJ38860 | |
| 48 | HMPREF9538_01820 | | ZP_08304144 | |
| 49 | t1924 | exo | NP_805691 | |
| 50 | pK2044_00525 | | YP_001687935 | plasmid pK2044 |

TABLE 11h
Data for Klebsiella, particularly K. pneumoniae (continued)

| No. | Start.Coord | End.Coord | Strand | Scaffold.ID |
|---|---|---|---|---|
| 1 | 4110458 | 4111246 | − | 640427102 |
| 2 | 1525960 | 1526820 | + | 642555218 |
| 3 | 4715 | 5554 | − | 648294624 |
| 4 | 34908 | 35255 | − | 640753021 |
| 5 | 36757 | 37062 | + | 641778223 |
| 6 | 1535907 | 1536932 | − | 651053122 |
| 7 | 159235 | 160521 | − | 651053122 |
| 8 | 2865 | 3848 | − | 647009817 |
| 9 | 3765814 | 3768501 | − | 646312028 |
| 10 | 67987 | 70674 | + | 643348532 |
| 11 | 4691 | 5551 | − | 647009817 |
| 12 | 2120 | 2368 | + | 648289812 |
| 13 | 8632 | 8931 | + | 640753081 |
| 14 | 12911 | 13663 | + | 647009752 |
| 15 | 20402 | 22975 | + | 647009874 |
| 16 | 93128 | 94558 | + | 647536396 |
| 17 | 3555 | 4568 | + | 638359041 |
| 18 | 14573 | 15328 | + | 647009752 |
| 19 | 933 | 1943 | − | 651332959 |
| 20 | 38514 | 39383 | + | 640753019 |
| 21 | 1563447 | 1563746 | + | 651053122 |
| 22 | 35802 | 36665 | + | 640753019 |
| 23 | 3648 | 4475 | − | 651332959 |
| 24 | 36662 | 37501 | + | 640753019 |
| 25 | 11204 | 11776 | + | 647009874 |
| 26 | 11773 | 12132 | + | 647009874 |
| 27 | 10265 | 10696 | + | 647009874 |
| 28 | 4537618 | 4539336 | + | 646564624 |
| 29 | 1561697 | 1562869 | + | 651053122 |
| 30 | 51251 | 51370 | − | 647536393 |
| 31 | 154 | 357 | − | 643042634 |
| 32 | 23022 | 23267 | + | 647009874 |
| 33 | 19408 | 19923 | + | 647009874 |
| 34 | 1552497 | 1552712 | + | 651053122 |
| 35 | 1551079 | 1551729 | + | 651053122 |
| 36 | 1550023 | 1551075 | + | 651053122 |
| 37 | 6165 | 7229 | + | 647009874 |
| 38 | 4543715 | 4546123 | − | 646564624 |
| 39 | 1555 | 2055 | − | 651333542 |
| 40 | 2380 | 3411 | − | 647009874 |
| 41 | 68933 | 70240 | + | 642555219 |
| 42 | 2637299 | 2637790 | + | 646312028 |
| 43 | 2932956 | 2933891 | + | 644736389 |
| 44 | 32101 | 32916 | − | 646311959 |
| 45 | 4084530 | 4085237 | − | 651053007 |
| 46 | 21137 | 21952 | − | 637000354 |
| 47 | 4067283 | 4067942 | + | 651053007 |
| 48 | 2070 | 2927 | − | 651333149 |
| 49 | 1967759 | 1968439 | + | 637000205 |
| 50 | 78317 | 81286 | − | 646564501 |

TABLE 12a
Data for Klebsiella, particularly K. pneumoniae

| | Scaffold.External. | | |
|---|---|---|---|
| No. | Accession | Scaffold.Name | best_pv |
| 1 | NC_007384 | Shigella sonnei Ss046: NC_007384 | 1.21395823682916e−119 |
| 2 | NC_011083 | Salmonella enterica subsp. enterica serovar Heidelberg str. SL476: | 1.70517984237646e−115 |
| | | NC_011083 | |
| 3 | NZ_ADWV01000045 | Escherichia coli MS 107-1 E_coliMS107-1-1.0.1_Cont44.1: | 3.12333368944279e−110 |
| | | NZ_ADWV01000045 | |
| 4 | NC_009651 | Klebsiella pneumoniae subsp. pneumoniae MGH 78578 plasmid pKPN5: | 1.8490215255217e−103 |
| | | NC_009651 | |
| 5 | NZ_ABFH01000001 | Salmonella enterica subsp. enterica serovar Virchow str. SL491, | 4.16211077482132e−97 |
| | | unfinished sequence: NZ_ABFH01000001 | |
| 6 | CP002910 | Klebsiella pneumoniae KCTC 2242: CP002910 | 1.34397014479151e−80 |
| 7 | CP002910 | Klebsiella pneumoniae KCTC 2242: CP002910 | 1.21342816034335e−71 |
| 8 | NZ_ACZD01000070 | Klebsiella pneumoniae subsp. rhinoscleromatis ATCC 13884 | 4.42089689687353e−61 |
| | | contig00070: NZ_ACZD01000070 | |
| 9 | NC_013850 | Klebsiella variicola At-22 chromosome: NC_013850 | 8.41004168548543e−61 |
| 10 | NC_011282 | Klebsiella pneumoniae 342 plasmid pKP187: NC_011282 | 8.41004168548543e−61 |
| 11 | NZ_ACZD01000070 | Klebsiella pneumoniae subsp. rhinoscleromatis ATCC 13884 | 8.41004168548543e−61 |
| | | contig00070: NZ_ACZD01000070 | |
| 12 | NZ_ADTP01000323 | Escherichia coli MS 69-1 E_coli69-1-1.0_Cont630.1: NZ_ADTP01000323 | 4.94539115664213e−60 |
| 13 | NC_009793 | Citrobacter koseri ATCC BAA-895 plasmid pCKO3: NC_009793 | 5.71878021049123e−59 |
| 14 | NZ_ACZD01000005 | Klebsiella pneumoniae subsp. rhinoscleromatis ATCC 13884 | 1.22977045856913e−58 |
| | | contig00005: NZ_ACZD01000005 | |
| 15 | NZ_ACZD01000127 | Klebsiella pneumoniae subsp. rhinoscleromatis ATCC 13884 | 1.49068550300511e−58 |
| | | contig00129: NZ_ACZD01000127 | |
| 16 | NZ_GG745515 | Klebsiella sp. 1_1_55 genomic scaffold supercont1.8: NZ_GG745515 | 7.09566707309011e−58 |
| 17 | NZ_AAJX01000089 | Escherichia coli B171, unfinished sequence: NZ_AAJX01000089 | 1.65409147045657e−56 |
| 18 | NZ_ACZD01000005 | Klebsiella pneumoniae subsp. rhinoscleromatis ATCC 13884 | 7.54090407593199e−55 |
| | | contig00005: NZ_ACZD01000005 | |
| 19 | NZ_AFBO01000042 | Klebsiella sp. MS 92-3 K_spMS92-3-l.0_Cont91.1: NZ_AFBO01000042 | 2.8348333685311e−54 |
| 20 | NC_009649 | Klebsiella pneumoniae subsp. pneumoniae MGH 78578 plasmid pKPN3: | 2.8348333685311e−54 |
| | | NC_009649 | |
| 21 | CP002910 | Klebsiella pneumoniae KCTC 2242: CP002910 | 2.83530891190408e−54 |
| 22 | NC_009649 | Klebsiella pneumoniae subsp. pneumoniae MGH 78578 plasmid pKPN3: | 4.89541334527925e−54 |
| | | NC_009649 | |
| 23 | NZ_AFBO01000042 | Klebsiella sp. MS 92-3 K_spMS92-3-l.0_Cont91.1: NZ_AFBO01000042 | 2.18592863711471e−53 |
| 24 | NC_009649 | Klebsiella pneumoniae subsp. pneumoniae MGH 78578 plasmid pKPN3: | 2.18592863711471e−53 |
| | | NC_009649 | |
| 25 | NZ_ACZD01000127 | Klebsiella pneumoniae subsp. rhinoscleromatis ATCC 13884 | 1.08576020331202e−52 |
| | | contig00129: NZ_ACZD01000127 | |
| 26 | NZ_ACZD01000127 | Klebsiella pneumoniae subsp. rhinoscleromatis ATCC 13884 | 1.37521017661703e−52 |
| | | contig00129: NZ_ACZD01000127 | |
| 27 | NZ_ACZD01000127 | Klebsiella pneumoniae subsp. rhinoscleromatis ATCC 13884 | 2.1362800317976e−52 |
| | | contig00129: NZ_ACZD01000127 | |
| 28 | NC_014121 | Enterobacter cloacae subsp. cloacae ATCC 13047 chromosome: | 2.21381524453565e−51 |
| | | NC_014121 | |
| 29 | CP002910 | Klebsiella pneumoniae KCTC 2242: CP002910 | 6.53846908249994e−51 |
| 30 | NZ_GG745512 | Klebsiella sp. 1_1_55 genomic scaffold supercont1.5: NZ_GG745512 | 2.78992804201171e−50 |
| 31 | NZ_CAAZ01000154 | Salmonella enterica subsp. enterica serovar Typhi str. E98-3139, | 4.10919139010377e−50 |
| | | unfinished sequence: NZ_CAAZ01000154 | |
| 32 | NZ_ACZD01000127 | Klebsiella pneumoniae subsp. rhinoscleromatis ATCC 13884 | 6.10670380866266e−50 |
| | | contig00129: NZ_ACZD01000127 | |
| 33 | NZ_ACZD01000127 | Klebsiella pneumoniae subsp. rhinoscleromatis ATCC 13884 | 1.00996840886331e−49 |
| | | contig00129: NZ_ACZD01000127 | |
| 34 | CP002910 | Klebsiella pneumoniae KCTC 2242: CP002910 | 1.33344542353295e−48 |
| 35 | CP002910 | Klebsiella pneumoniae KCTC 2242: CP002910 | 3.00269800428115e−48 |
| 36 | CP002910 | Klebsiella pneumoniae KCTC 2242: CP002910 | 3.13126971411145e−48 |
| 37 | NZ_ACZD01000127 | Klebsiella pneumoniae subsp. rhinoscleromatis ATCC 13884 | 3.13126971411145e−48 |
| | | contig00129: NZ_ACZD01000127 | |
| 38 | NC_014121 | Enterobacter cloacae subsp. cloacae ATCC 13047 chromosome: | 3.79149056816214e−48 |
| | | NC_014121 | |
| 39 | NZ_AFBO01000625 | Klebsiella sp. MS 92-3 K_spMS92-3-1.0_Cont1175.2: NZ_AFBO01000625 | 2.5975548233422e−47 |
| 40 | NZ_ACZD01000127 | Klebsiella pneumoniae subsp. rhinoscleromatis ATCC 13884 | 1.05391699509183e−46 |
| | | contig00129: NZ_ACZD01000127 | |
| 41 | NC_011092 | Salmonella enterica subsp. enterica serovar Schwarzengrund str. | 4.42178242563847e−46 |
| | | CVM19633 plasmid pCVM19633_110: NC_011092 | |
| 42 | NC_013850 | Klebsiella variicola At-22 chromosome: NC_013850 | 6.65483065085068e−46 |
| 43 | NC_012912 | Dickeya zeae Ech1591: NC_012912 | 1.05262166415392e−45 |
| 44 | NC_013509 | Edwardsiella tarda EIB202 plasmid pEIB202: NC_013509 | 1.24074600175825e−45 |
| 45 | AP011957 | Salmonella enterica subsp. enterica serovar Typhimurium str. | 2.46270503642371e−45 |
| | | T000240 DNA: AP011957 | |
| 46 | NC_006856 | Salmonella enterica subsp. enterica serovar Choleraesuis str. SC- | 9.02631418377704e−45 |
| | | B67 plasmid pSC138: NC_006856 | |
| 47 | AP011957 | Salmonella enterica subsp. enterica serovar Typhimurium str. | 3.37821430101762e−44 |
| | | T000240 DNA: AP011957 | |
| 48 | NZ_AFBO01000232 | Klebsiella sp. MS 92-3 K_spMS92-3-1.0_Cont505.1: NZ_AFBO01000232 | 4.87973844618098e−43 |
| 49 | NC_004631 | Salmonella enterica subsp. enterica serovar Typhi Ty2: NC_004631 | 1.54269244019591e−42 |
| 50 | NC_006625 | Klebsiella pneumoniae NTUH-K2044 plasmid pK2044: NC_006625 | 1.89874529968327e−42 |

TABLE 12b
Data for Klebsiella, particularly K. pneumoniae (continued)

| No. | sign_phenos | sign_phenos_class | best_pheno | best_pheno_class |
|---|---|---|---|---|
| 1 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 2 | A/S; AUG; AZT; CAX; CAZ; CFT; CP; CPE; | aminoglycoside; fluoroquinolone; lactam; other | AUG | lactam |
| | CRM; ETP; GM; LVX; P/T; TO; T/S | | | |
| 3 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 4 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 5 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 6 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 7 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 8 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 9 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 10 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 11 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 12 | A/S; AUG; AZT; CAX; CAZ; CFT; CP; CPE; | aminoglycoside; fluoroquinolone; lactam; other | CAZ | lactam |
| | CRM; ETP; GM; LVX; P/T; TO; T/S | | | |
| 13 | A/S; AUG; AZT; CAX; CAZ; CFT; CP; CPE; | aminoglycoside; fluoroquinolone; lactam; other | AUG | lactam |
| | CRM; ETP; GM; LVX; P/T; TO; T/S | | | |
| 14 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 15 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 16 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 17 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 18 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 19 | A/S; AUG; AZT; CAX; CAZ; CFT; CP; CPE; | aminoglycoside; fluoroquinolone; lactam; other | P/T | lactam |
| | CRM; ETP; GM; LVX; P/T; TO; T/S | | | |
| 20 | A/S; AUG; AZT; CAX; CAZ; CFT; CP; CPE; | aminoglycoside; fluoroquinolone; lactam; other | P/T | lactam |
| | CRM; ETP; GM; LVX; P/T; TO; T/S | | | |
| 21 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 22 | A/S; AUG; AZT; CAX; CAZ; CFT; CP; CPE; | aminoglycoside; fluoroquinolone; lactam; other | P/T | lactam |
| | CRM; ETP; GM; LVX; P/T; TO; T/S | | | |
| 23 | A/S; AUG; AZT; CAX; CAZ; CFT; CP; CPE; | aminoglycoside; fluoroquinolone; lactam; other | P/T | lactam |
| | CRM; ETP; GM; LVX; P/T; TO; T/S | | | |
| 24 | A/S; AUG; AZT; CAX; CAZ; CFT; CP; CPE; | aminoglycoside; fluoroquinolone; lactam; other | P/T | lactam |
| | CRM; ETP; GM; LVX; P/T; TO; T/S | | | |
| 25 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 26 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 27 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 28 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 29 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 30 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 31 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 32 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 33 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 34 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 35 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 36 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 37 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 38 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 39 | A/S; AUG; AZT; CAX; CAZ; CFT; CP; CPE; | aminoglycoside; fluoroquinolone; lactam; other | T/S | other |
| | CRM; GM; LVX; P/T; TO; T/S | | | |
| 40 | A/S; AUG; AZT; CAX; CAZ; CFT; CP; CPE; | aminoglycoside; fluoroquinolone; lactam; other | CP | fluoroquinolone |
| | CRM; ETP; GM; LVX; P/T; TO; T/S | | | |
| 41 | A/S; AUG; AZT; CAX; CAZ; CFT; CP; CPE; | aminoglycoside; fluoroquinolone; lactam; other | AUG | lactam |
| | CRM; ETP; GM; LVX; P/T; TO; T/S | | | |
| 42 | AUG; AZT; CAX; CAZ; CFT; CP; CPE; CRM; | aminoglycoside; fluoroquinolone; lactam; other | ETP | lactam |
| | ETP; LVX; P/T; TO; T/S | | | |
| 43 | AUG; AZT; CAX; CAZ; CFT; CP; CPE; CRM; | aminoglycoside; fluoroquinolone; lactam; other | ETP | lactam |
| | ETP; LVX; P/T; TO; T/S | | | |
| 44 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 45 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 46 | A/S; AUG; AZT; CAX; CAZ; CFT; CP; CPE; | aminoglycoside; fluoroquinolone; lactam; other | TO | aminoglycoside |
| | CRM; ETP; GM; LVX; P/T; TO; T/S | | | |
| 47 | A/S; AUG; AZT; CAX; CAZ; CFT; CP; CPE; | aminoglycoside; fluoroquinolone; lactam; other | AUG | lactam |
| | CRM; ETP; GM; LVX; P/T; TO; T/S | | | |
| 48 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |
| 49 | A/S; AUG; AZT; CAX; CAZ; CFT; CP; CPE; | aminoglycoside; fluoroquinolone; lactam; other | ETP | lactam |
| | CRM; ETP; GM; LVX; P/T; TO; T/S | | | |
| 50 | A/S; AUG; AZT; CAX; CAZ; CFT; CP; CPE; | aminoglycoside; fluoroquinolone; lactam; other | T/S | other |
| | CRM; ETP; GM; LVX; P/T; TO; T/S | | | |

TABLE 12c
Data for Klebsiella, particularly K. pneumoniae (continued)

| No. | num_aminoglycoside | num_fluoroquinolone | num_lactam | num_other |
|---|---|---|---|---|
| 1 | 2 | 2 | 10 | 1 |
| 2 | 2 | 2 | 10 | 1 |
| 3 | 2 | 2 | 10 | 1 |
| 4 | 2 | 2 | 10 | 1 |
| 5 | 2 | 2 | 10 | 1 |
| 6 | 2 | 2 | 10 | 1 |
| 7 | 2 | 2 | 10 | 1 |
| 8 | 2 | 2 | 10 | 1 |
| 9 | 2 | 2 | 10 | 1 |
| 10 | 2 | 2 | 10 | 1 |
| 11 | 2 | 2 | 10 | 1 |
| 12 | 2 | 2 | 10 | 1 |
| 13 | 2 | 2 | 10 | 1 |
| 14 | 2 | 2 | 10 | 1 |
| 15 | 2 | 2 | 10 | 1 |
| 16 | 2 | 2 | 10 | 1 |
| 17 | 2 | 2 | 10 | 1 |
| 18 | 2 | 2 | 10 | 1 |
| 19 | 2 | 2 | 10 | 1 |
| 20 | 2 | 2 | 10 | 1 |
| 21 | 2 | 2 | 10 | 1 |
| 22 | 2 | 2 | 10 | 1 |
| 23 | 2 | 2 | 10 | 1 |
| 24 | 2 | 2 | 10 | 1 |
| 25 | 2 | 2 | 10 | 1 |
| 26 | 2 | 2 | 10 | 1 |
| 27 | 2 | 2 | 10 | 1 |
| 28 | 2 | 2 | 10 | 1 |
| 29 | 2 | 2 | 10 | 1 |
| 30 | 2 | 2 | 10 | 1 |
| 31 | 2 | 2 | 10 | 1 |
| 32 | 2 | 2 | 10 | 1 |
| 33 | 2 | 2 | 10 | 1 |
| 34 | 2 | 2 | 10 | 1 |
| 35 | 2 | 2 | 10 | 1 |
| 36 | 2 | 2 | 10 | 1 |
| 37 | 2 | 2 | 10 | 1 |
| 38 | 2 | 2 | 10 | 1 |
| 39 | 2 | 2 | 9 | 1 |
| 40 | 2 | 2 | 10 | 1 |
| 41 | 2 | 2 | 10 | 1 |
| 42 | 1 | 2 | 9 | 1 |
| 43 | 1 | 2 | 9 | 1 |
| 44 | 2 | 2 | 10 | 1 |
| 45 | 2 | 2 | 10 | 1 |
| 46 | 2 | 2 | 10 | 1 |
| 47 | 2 | 2 | 10 | 1 |
| 48 | 2 | 2 | 10 | 1 |
| 49 | 2 | 2 | 10 | 1 |
| 50 | 2 | 2 | 10 | 1 |

TABLE 12d
Data for Klebsiella, particularly K. pneumoniae (continued)

| No. | A/S_pv_adj | AUG_pv_adj | AZT_pv_adj | CAX_pv_adj | CAZ_pv_adj |
|---|---|---|---|---|---|
| 1 | 1.28E−76 | 1.24E−105 | 4.06E−95 | 3.39E−95 | 5.99E−93 |
| 2 | 1.18E−98 | 1.71E−115 | 9.09E−91 | 6.55E−88 | 1.66E−96 |
| 3 | 1.08E−81 | 5.85E−86 | 2.76E−94 | 4.16E−97 | 6.84E−100 |
| 4 | 5.55E−76 | 3.05E−84 | 1.38E−91 | 2.64E−96 | 5.33E−94 |
| 5 | 1.22E−70 | 3.41E−71 | 1.41E−80 | 1.50E−83 | 6.22E−90 |
| 6 | 7.70E−21 | 1.03E−41 | 8.20E−48 | 2.50E−44 | 2.69E−46 |
| 7 | 8.54E−16 | 4.34E−29 | 4.54E−38 | 5.50E−34 | 1.34E−34 |
| 8 | 2.62E−12 | 1.74E−24 | 1.49E−26 | 2.74E−25 | 2.84E−24 |
| 9 | 5.20E−12 | 2.50E−24 | 3.69E−26 | 7.31E−25 | 7.66E−24 |
| 10 | 5.20E−12 | 2.50E−24 | 3.69E−26 | 7.31E−25 | 7.66E−24 |
| 11 | 5.20E−12 | 2.50E−24 | 3.69E−26 | 7.31E−25 | 7.66E−24 |
| 12 | 3.32E−43 | 1.75E−59 | 1.48E−54 | 9.28E−55 | 4.95E−60 |
| 13 | 2.41E−43 | 5.72E−59 | 4.20E−53 | 2.96E−53 | 2.14E−58 |
| 14 | 2.47E−16 | 8.57E−27 | 1.49E−34 | 1.96E−31 | 8.83E−33 |
| 15 | 1.72E−35 | 6.40E−55 | 1.25E−44 | 4.12E−43 | 1.61E−46 |
| 16 | 3.04E−18 | 5.19E−32 | 4.83E−36 | 2.23E−35 | 1.91E−32 |
| 17 | 1.02E−47 | 9.19E−45 | 1.85E−48 | 9.17E−53 | 6.18E−56 |
| 18 | 3.51E−17 | 3.04E−23 | 8.29E−30 | 1.21E−27 | 9.29E−29 |
| 19 | 1.03E−40 | 7.90E−52 | 1.41E−37 | 4.81E−39 | 9.97E−38 |
| 20 | 1.03E−40 | 7.90E−52 | 1.41E−37 | 4.81E−39 | 9.97E−38 |
| 21 | 8.56E−34 | 4.84E−53 | 8.75E−43 | 2.41E−41 | 9.43E−45 |
| 22 | 5.13E−42 | 1.10E−52 | 3.02E−38 | 9.55E−40 | 3.15E−38 |
| 23 | 1.11E−41 | 3.84E−52 | 1.00E−37 | 3.11E−39 | 8.03E−38 |
| 24 | 1.11E−41 | 3.84E−52 | 1.00E−37 | 3.11E−39 | 8.03E−38 |
| 25 | 8.85E−33 | 1.54E−51 | 2.83E−42 | 1.24E−40 | 2.88E−44 |
| 26 | 1.96E−32 | 2.54E−52 | 2.98E−42 | 1.41E−40 | 3.55E−44 |
| 27 | 5.12E−32 | 6.70E−50 | 1.84E−40 | 2.34E−38 | 1.33E−42 |
| 28 | 1.65E−30 | 3.00E−47 | 1.96E−38 | 1.37E−36 | 4.94E−39 |
| 29 | 4.43E−31 | 1.45E−47 | 2.29E−39 | 1.06E−37 | 3.63E−40 |
| 30 | 6.99E−12 | 2.37E−26 | 2.77E−24 | 5.28E−24 | 2.55E−26 |
| 31 | 2.78E−31 | 7.62E−46 | 1.11E−38 | 5.40E−37 | 1.95E−39 |
| 32 | 9.62E−32 | 1.45E−47 | 2.29E−39 | 1.06E−37 | 3.63E−40 |
| 33 | 2.27E−31 | 2.28E−47 | 3.20E−39 | 1.66E−37 | 5.77E−40 |
| 34 | 9.74E−28 | 9.40E−43 | 1.44E−35 | 7.30E−34 | 3.24E−36 |
| 35 | 1.57E−28 | 2.77E−42 | 6.21E−36 | 1.59E−34 | 2.14E−36 |
| 36 | 3.41E−27 | 2.34E−43 | 7.21E−36 | 4.53E−33 | 9.07E−37 |
| 37 | 3.41E−27 | 2.34E−43 | 7.21E−36 | 4.53E−33 | 9.07E−37 |
| 38 | 2.00E−30 | 7.61E−47 | 1.00E−38 | 1.06E−37 | 3.63E−40 |
| 39 | 8.29E−47 | 2.48E−33 | 3.67E−37 | 5.57E−42 | 2.74E−43 |
| 40 | 1.53E−27 | 6.97E−43 | 2.91E−36 | 2.70E−35 | 5.96E−38 |
| 41 | 1.75E−44 | 4.42E−46 | 2.16E−37 | 1.21E−36 | 1.12E−42 |
| 42 | 4.51E−02 | 4.66E−12 | 9.77E−17 | 3.13E−16 | 9.90E−15 |
| 43 | 5.37E−02 | 5.73E−12 | 1.27E−16 | 6.11E−16 | 1.15E−14 |
| 44 | 6.65E−31 | 4.17E−32 | 1.40E−25 | 3.69E−27 | 1.78E−29 |
| 45 | 6.15E−34 | 5.78E−41 | 2.29E−36 | 9.94E−38 | 1.48E−38 |
| 46 | 3.01E−32 | 3.12E−38 | 7.22E−37 | 3.96E−36 | 6.00E−37 |
| 47 | 4.35E−41 | 3.38E−44 | 1.49E−30 | 1.42E−33 | 1.82E−29 |
| 48 | 6.41E−31 | 1.63E−41 | 3.24E−33 | 2.26E−36 | 1.97E−31 |
| 49 | 2.54E−11 | 2.61E−18 | 8.71E−21 | 1.23E−18 | 1.87E−19 |
| 50 | 2.38E−23 | 3.76E−29 | 2.21E−27 | 4.97E−29 | 1.37E−29 |

TABLE 12e
Data for Klebsiella, particularly K. pneumoniae (continued)

| No. | CFT_pv_adj | CP_pv_adj | CPE_pv_adj | CRM_pv_adj | ETP_pv_adj |
|---|---|---|---|---|---|
| 1 | 2.17E−99 | 2.40E−85 | 2.31E−68 | 1.21E−82 | 1.87E−58 |
| 2 | 2.52E−83 | 1.28E−61 | 4.61E−66 | 7.83E−57 | 8.20E−51 |
| 3 | 3.93E−93 | 2.17E−58 | 2.89E−43 | 3.32E−73 | 3.54E−24 |
| 4 | 1.52E−90 | 4.25E−60 | 1.75E−43 | 2.59E−71 | 1.63E−23 |
| 5 | 3.61E−77 | 1.76E−47 | 1.47E−36 | 5.14E−62 | 6.31E−21 |
| 6 | 2.84E−46 | 1.53E−54 | 2.89E−63 | 1.06E−36 | 1.34E−80 |
| 7 | 8.43E−36 | 1.96E−43 | 3.06E−55 | 3.95E−29 | 1.21E−71 |
| 8 | 5.55E−27 | 2.63E−33 | 6.90E−40 | 1.01E−20 | 4.42E−61 |
| 9 | 1.58E−26 | 5.49E−33 | 1.32E−39 | 2.40E−20 | 8.41E−61 |
| 10 | 1.58E−26 | 5.49E−33 | 1.32E−39 | 2.40E−20 | 8.41E−61 |
| 11 | 1.58E−26 | 5.49E−33 | 1.32E−39 | 4.98E−21 | 8.41E−61 |
| 12 | 4.39E−54 | 4.96E−60 | 1.80E−51 | 7.75E−50 | 7.02E−53 |
| 13 | 1.26E−52 | 1.35E−58 | 1.72E−50 | 4.57E−49 | 3.71E−52 |
| 14 | 1.28E−31 | 2.00E−30 | 6.27E−42 | 1.20E−29 | 1.23E−58 |
| 15 | 2.05E−41 | 2.56E−52 | 7.76E−46 | 3.74E−32 | 5.20E−48 |
| 16 | 1.52E−36 | 2.10E−37 | 5.58E−48 | 5.62E−28 | 7.10E−58 |
| 17 | 9.26E−49 | 4.16E−22 | 1.22E−19 | 9.42E−36 | 5.55E−05 |
| 18 | 6.27E−28 | 1.02E−25 | 4.83E−36 | 7.62E−29 | 7.54E−55 |
| 19 | 9.92E−40 | 2.65E−39 | 2.28E−41 | 4.71E−28 | 1.10E−32 |
| 20 | 9.92E−40 | 2.65E−39 | 2.28E−41 | 4.71E−28 | 1.10E−32 |
| 21 | 1.47E−40 | 1.18E−49 | 1.74E−45 | 2.18E−32 | 1.46E−46 |
| 22 | 2.00E−40 | 6.22E−40 | 7.04E−42 | 1.08E−28 | 5.10E−33 |
| 23 | 6.77E−40 | 2.32E−39 | 3.78E−41 | 2.22E−28 | 3.70E−32 |
| 24 | 6.77E−40 | 2.32E−39 | 3.78E−41 | 2.22E−28 | 3.70E−32 |
| 25 | 4.69E−40 | 3.62E−49 | 1.06E−44 | 1.70E−33 | 2.11E−45 |
| 26 | 4.89E−40 | 1.97E−49 | 1.88E−45 | 2.34E−32 | 8.59E−46 |
| 27 | 5.94E−39 | 1.13E−47 | 2.46E−43 | 3.44E−33 | 1.93E−49 |
| 28 | 3.32E−36 | 2.21E−51 | 1.87E−44 | 1.56E−31 | 1.83E−41 |
| 29 | 2.69E−37 | 6.54E−51 | 4.48E−44 | 2.19E−30 | 3.44E−41 |
| 30 | 1.39E−24 | 6.75E−31 | 1.12E−35 | 5.11E−15 | 2.79E−50 |
| 31 | 1.34E−36 | 4.11E−50 | 1.67E−43 | 1.45E−31 | 8.85E−41 |
| 32 | 2.69E−37 | 6.11E−50 | 4.48E−44 | 2.19E−30 | 3.44E−41 |
| 33 | 6.74E−37 | 1.01E−49 | 6.91E−44 | 1.07E−30 | 4.69E−41 |
| 34 | 3.28E−34 | 1.33E−48 | 1.41E−40 | 1.52E−30 | 7.40E−43 |
| 35 | 3.72E−36 | 3.00E−48 | 1.67E−40 | 8.05E−31 | 2.56E−43 |
| 36 | 1.04E−34 | 3.13E−48 | 5.88E−41 | 1.06E−29 | 3.84E−43 |
| 37 | 1.04E−34 | 3.13E−48 | 5.88E−41 | 1.06E−29 | 3.84E−43 |
| 38 | 2.69E−37 | 3.79E−48 | 4.21E−43 | 9.89E−32 | 5.95E−40 |
| 39 | 2.84E−38 | 3.78E−14 | 1.15E−05 | 9.70E−31 | 9.25E−01 |
| 40 | 1.88E−36 | 1.05E−46 | 3.80E−41 | 1.01E−30 | 2.77E−43 |
| 41 | 4.01E−36 | 1.78E−19 | 2.93E−16 | 1.27E−21 | 2.46E−10 |
| 42 | 5.45E−16 | 4.08E−18 | 6.25E−28 | 7.56E−08 | 6.65E−46 |
| 43 | 1.13E−15 | 5.57E−18 | 9.82E−28 | 1.16E−07 | 1.05E−45 |
| 44 | 2.55E−25 | 6.58E−14 | 7.47E−22 | 3.89E−16 | 2.18E−20 |
| 45 | 5.14E−35 | 2.41E−27 | 5.02E−15 | 4.61E−29 | 2.22E−12 |
| 46 | 1.40E−36 | 2.78E−33 | 1.06E−29 | 5.12E−30 | 2.25E−28 |
| 47 | 3.46E−32 | 1.18E−32 | 1.62E−32 | 2.49E−29 | 1.62E−26 |
| 48 | 6.74E−36 | 1.36E−34 | 2.38E−34 | 1.27E−25 | 6.79E−29 |
| 49 | 9.64E−20 | 3.40E−22 | 1.36E−30 | 8.87E−18 | 1.54E−42 |
| 50 | 1.19E−26 | 1.97E−18 | 9.42E−19 | 1.46E−19 | 1.52E−12 |

TABLE 12f
Data for Klebsiella, particularly K. pneumoniae (continued)

| No. | GM_pv_adj | LVX_pv_adj | P/T_pv_adj | TO_pv_adj | T/S_pv_adj |
|---|---|---|---|---|---|
| 1 | 6.24E−60 | 3.97E−80 | 7.18E−96 | 1.21E−119 | 1.12E−106 |
| 2 | 6.10E−67 | 3.76E−55 | 1.20E−67 | 2.77E−105 | 3.59E−83 |
| 3 | 6.00E−68 | 3.14E−50 | 5.58E−70 | 1.37E−91 | 3.12E−110 |
| 4 | 3.06E−63 | 4.39E−52 | 7.17E−70 | 6.65E−91 | 1.85E−103 |
| 5 | 5.29E−59 | 6.80E−39 | 4.00E−51 | 1.40E−82 | 4.16E−97 |
| 6 | 5.64E−09 | 3.19E−59 | 3.01E−52 | 7.11E−48 | 1.50E−31 |
| 7 | 1.61E−05 | 6.12E−48 | 2.20E−40 | 1.45E−34 | 2.10E−20 |
| 8 | 1.57E−06 | 6.85E−37 | 6.06E−35 | 1.79E−29 | 2.02E−18 |
| 9 | 2.46E−06 | 1.41E−36 | 1.30E−34 | 3.62E−29 | 2.62E−18 |
| 10 | 2.46E−06 | 1.41E−36 | 1.30E−34 | 3.62E−29 | 2.62E−18 |
| 11 | 2.46E−06 | 1.41E−36 | 1.30E−34 | 3.62E−29 | 2.62E−18 |
| 12 | 2.30E−25 | 1.32E−53 | 6.01E−52 | 5.28E−59 | 5.07E−39 |
| 13 | 1.61E−25 | 2.14E−52 | 1.19E−50 | 1.22E−58 | 6.30E−38 |
| 14 | 6.89E−05 | 6.38E−32 | 9.66E−29 | 7.19E−31 | 3.05E−15 |
| 15 | 1.55E−23 | 1.89E−48 | 1.36E−48 | 1.49E−58 | 1.23E−46 |
| 16 | 7.14E−10 | 1.99E−38 | 1.30E−34 | 9.13E−37 | 1.47E−21 |
| 17 | 6.27E−40 | 9.96E−18 | 4.98E−31 | 1.50E−43 | 1.65E−56 |
| 18 | 7.35E−05 | 4.57E−28 | 1.61E−25 | 2.68E−27 | 2.50E−15 |
| 19 | 1.16E−19 | 5.28E−36 | 2.83E−54 | 2.59E−42 | 9.74E−48 |
| 20 | 1.16E−19 | 5.28E−36 | 2.83E−54 | 2.59E−42 | 9.74E−48 |
| 21 | 6.44E−22 | 5.93E−45 | 3.28E−47 | 2.84E−54 | 2.84E−46 |
| 22 | 6.19E−20 | 1.49E−36 | 4.90E−54 | 5.19E−43 | 1.61E−47 |
| 23 | 4.80E−20 | 5.67E−36 | 2.19E−53 | 1.90E−42 | 7.35E−49 |
| 24 | 1.78E−19 | 5.67E−36 | 2.19E−53 | 1.90E−42 | 6.83E−48 |
| 25 | 1.71E−21 | 3.92E−44 | 2.20E−46 | 1.09E−52 | 8.26E−45 |
| 26 | 1.49E−20 | 1.39E−44 | 1.02E−46 | 1.38E−52 | 9.52E−46 |
| 27 | 9.23E−19 | 4.68E−44 | 1.13E−48 | 2.14E−52 | 7.18E−42 |
| 28 | 1.04E−18 | 2.43E−47 | 2.68E−42 | 8.73E−48 | 1.22E−43 |
| 29 | 1.81E−19 | 6.48E−47 | 5.36E−42 | 7.69E−49 | 5.28E−43 |
| 30 | 1.67E−04 | 1.19E−33 | 1.48E−27 | 1.33E−24 | 2.01E−19 |
| 31 | 2.71E−19 | 3.53E−46 | 2.81E−41 | 2.57E−47 | 3.11E−43 |
| 32 | 3.53E−20 | 6.26E−46 | 5.36E−42 | 7.69E−49 | 5.57E−44 |
| 33 | 4.08E−20 | 9.51E−46 | 1.44E−41 | 1.11E−48 | 8.48E−44 |
| 34 | 9.00E−16 | 3.30E−45 | 1.53E−41 | 3.99E−46 | 1.50E−39 |
| 35 | 4.69E−14 | 4.82E−45 | 1.45E−39 | 3.68E−47 | 5.85E−39 |
| 36 | 6.72E−15 | 8.68E−45 | 4.34E−42 | 8.00E−45 | 1.33E−38 |
| 37 | 6.72E−15 | 8.68E−45 | 4.34E−42 | 8.00E−45 | 1.33E−38 |
| 38 | 4.79E−19 | 4.19E−45 | 5.36E−42 | 3.42E−47 | 5.28E−43 |
| 39 | 1.94E−40 | 1.06E−10 | 6.27E−21 | 4.23E−35 | 2.60E−47 |
| 40 | 1.53E−15 | 3.13E−43 | 2.72E−42 | 3.02E−45 | 1.22E−39 |
| 41 | 9.94E−38 | 3.55E−16 | 4.46E−22 | 7.61E−44 | 1.24E−41 |
| 42 | 2.34E−02 | 4.47E−21 | 3.44E−16 | 3.10E−15 | 2.24E−09 |
| 43 | 2.39E−02 | 1.02E−20 | 4.32E−16 | 6.38E−15 | 3.86E−09 |
| 44 | 3.44E−22 | 5.51E−13 | 6.56E−18 | 4.39E−24 | 1.24E−45 |
| 45 | 1.36E−21 | 1.14E−24 | 2.38E−33 | 2.46E−45 | 1.53E−29 |
| 46 | 1.36E−19 | 4.39E−32 | 1.03E−35 | 9.03E−45 | 5.01E−44 |
| 47 | 1.18E−14 | 5.29E−30 | 3.76E−40 | 3.55E−44 | 5.56E−38 |
| 48 | 4.91E−17 | 8.23E−33 | 1.06E−40 | 6.11E−39 | 4.88E−43 |
| 49 | 1.35E−04 | 2.25E−22 | 1.78E−19 | 5.28E−22 | 2.63E−12 |
| 50 | 8.68E−26 | 1.45E−15 | 1.73E−22 | 6.68E−34 | 1.90E−42 |

TABLE 12g
Data for Klebsiella, particularly K. pneumoniae (continued)

| No. | Locus.Tag | Gene.Symbol | GenBank.Accession | Chromosome |
|---|---|---|---|---|
| 1 | SSON_3896 | aadA | YP_312670 | |
| 2 | SeHA_C1580 | blaT | YP_002045448 | |
| 3 | HMPREF9345_05066 | | ZP_07100147 | |
| 4 | KPN_pKPN5p08201 | | YP_001338811 | plasmid pKPN5 |
| 5 | Salmoentericaenterica_010100000245 | | ZP_02702077 | |
| 6 | KPN2242_07235 | | AEJ97365 | |
| 7 | KPN2242_00750 | | AEJ96075 | |
| 8 | HMPREF0484_1390 | mgtA | ZP_06014374 | |
| 9 | Kvar_3592 | | YP_003440504 | |
| 10 | KPK_A0103 | | YP_002235751 | plasmid pKP187 |
| 11 | HMPREF0484_1392 | pacL | ZP_06014376 | |
| 12 | HMPREF9534_04427 | | ZP_07188952 | |
| 13 | CKO_pCKO3p06155 | | YP_001456633 | plasmid pCKO3 |
| 14 | HMPREF0484_0044 | vmtV | ZP_06013029 | |
| 15 | HMPREF0484_2697 | | ZP_06015679 | |
| 16 | HMPREF0485_04762 | | ZP_06552358 | |
| 17 | EcolB_01004576 | | ZP_00708527 | |
| 18 | HMPREF0484_0046 | | ZP_06013031 | |
| 19 | HMPREF9538_00440 | | ZP_08302804 | |
| 20 | KPN_pKPN3p05893 | ptxE | YP_001338502 | plasmid pKPN3 |
| 21 | KPN2242_07425 | | AEJ97403 | |
| 22 | KPN_pKPN3p05890 | ptxB | YP_001338499 | plasmid pKPN3 |
| 23 | HMPREF9538_00443 | | ZP_08302807 | |
| 24 | KPN_pKPN3p05891 | ptxC | YP_001338500 | plasmid pKPN3 |
| 25 | HMPREF0484_2686 | | ZP_06015668 | |
| 26 | HMPREF0484_2687 | gpw | ZP_06015669 | |
| 27 | HMPREF0484_2684 | | ZP_06015666 | |
| 28 | ECL_04426 | | YP_003614904 | |
| 29 | KPN2242_07415 | | AEJ97401 | |
| 30 | HMPREF0485_03923 | | ZP_06551519 | |
| 31 | Salmonellentericaenterica_010100016432 | | ZP_03412648 | |
| 32 | HMPREF0484_2698 | | ZP_06015680 | |
| 33 | HMPREF0484_2694 | fii | ZP_06015676 | |
| 34 | KPN2242_07345 | | AEJ97387 | |
| 35 | KPN2242_07330 | | AEJ97384 | |
| 36 | KPN2242_07325 | | AEJ97383 | |
| 37 | HMPREF0484_2676 | | ZP_06015658 | |
| 38 | ECL_04434 | | YP_003614912 | |
| 39 | HMPREF9538_04737 | | ZP_08307036 | |
| 40 | HMPREF0484_2673 | | ZP_06015655 | |
| 41 | SeSA_B0079 | | YP_002112958 | plasmid |
| | | | | pCVM19633_110 |
| 42 | Kvar_2527 | | YP_003439449 | |
| 43 | Dd1591_2579 | | YP_003004893 | |
| 44 | ETAE_p041 | folP | YP_003297635 | plasmid pEIB202 |
| 45 | STMDT12_C39340 | | BAJ38877 | |
| 46 | SC026 | aph | YP_209331 | plasmid pSC138 |
| 47 | STMDT12_C39170 | | BAJ38860 | |
| 48 | HMPREF9538_01820 | | ZP_08304144 | |
| 49 | t1924 | exo | NP_805691 | |
| 50 | pK2044_00525 | | YP_001687935 | plasmid pK2044 |

TABLE 12h
Data for Klebsiella, particularly K. pneumoniae (continued)

| No. | Start.Coord | End.Coord | Strand | Scaffold.ID |
|---|---|---|---|---|
| 1 | 4110458 | 4111246 | − | 640427102 |
| 2 | 1525960 | 1526820 | + | 642555218 |
| 3 | 4715 | 5554 | − | 648294624 |
| 4 | 34908 | 35255 | − | 640753021 |
| 5 | 36757 | 37062 | + | 641778223 |
| 6 | 1535907 | 1536932 | − | 651053122 |
| 7 | 159235 | 160521 | − | 651053122 |
| 8 | 2865 | 3848 | − | 647009817 |
| 9 | 3765814 | 3768501 | − | 646312028 |
| 10 | 67987 | 70674 | + | 643348532 |
| 11 | 4691 | 5551 | − | 647009817 |
| 12 | 2120 | 2368 | + | 648289812 |
| 13 | 8632 | 8931 | + | 640753081 |
| 14 | 12911 | 13663 | + | 647009752 |
| 15 | 20402 | 22975 | + | 647009874 |
| 16 | 93128 | 94558 | + | 647536396 |
| 17 | 3555 | 4568 | + | 638359041 |
| 18 | 14573 | 15328 | + | 647009752 |
| 19 | 933 | 1943 | − | 651332959 |
| 20 | 38514 | 39383 | + | 640753019 |
| 21 | 1563447 | 1563746 | + | 651053122 |
| 22 | 35802 | 36665 | + | 640753019 |
| 23 | 3648 | 4475 | − | 651332959 |
| 24 | 36662 | 37501 | + | 640753019 |
| 25 | 11204 | 11776 | + | 647009874 |
| 26 | 11773 | 12132 | + | 647009874 |
| 27 | 10265 | 10696 | + | 647009874 |
| 28 | 4537618 | 4539336 | + | 646564624 |
| 29 | 1561697 | 1562869 | + | 651053122 |
| 30 | 51251 | 51370 | − | 647536393 |
| 31 | 154 | 357 | − | 643042634 |
| 32 | 23022 | 23267 | + | 647009874 |
| 33 | 19408 | 19923 | + | 647009874 |
| 34 | 1552497 | 1552712 | + | 651053122 |
| 35 | 1551079 | 1551729 | + | 651053122 |
| 36 | 1550023 | 1551075 | + | 651053122 |
| 37 | 6165 | 7229 | + | 647009874 |
| 38 | 4543715 | 4546123 | − | 646564624 |
| 39 | 1555 | 2055 | − | 651333542 |
| 40 | 2380 | 3411 | − | 647009874 |
| 41 | 68933 | 70240 | + | 642555219 |
| 42 | 2637299 | 2637790 | + | 646312028 |
| 43 | 2932956 | 2933891 | + | 644736389 |
| 44 | 32101 | 32916 | − | 646311959 |
| 45 | 4084530 | 4085237 | − | 651053007 |
| 46 | 21137 | 21952 | − | 637000354 |
| 47 | 4067283 | 4067942 | + | 651053007 |
| 48 | 2070 | 2927 | − | 651333149 |
| 49 | 1967759 | 1968439 | + | 637000205 |
| 50 | 78317 | 81286 | − | 646564501 |

Example 7: Proteus, particularly Proteus mirabilis, Proteus penneri and/or Proteus vulgaris

The procedure was carried out as in Example 1, except that the following microorganisms were used:

Bacterial Strains

The inventors selected 582 Proteus strains from the microbiology strain collection at Siemens Healthcare Diagnostics (West Sacramento, Calif.) for susceptibility testing and whole genome sequencing.

From MetaRef, 2318 centroids of Proteus species were used as reference sequences.

The results for Proteus are shown in Tables 13 (corresponding to Table 1) and 14 (corresponding to Table 2).

TABLE 13a
Data for Proteus species

| | Scaffold.External. | | |
|---|---|---|---|
| No. | Accession | Scaffold.Name | best_pv |
| 1 | NZ_ABVP01000020 | Proteus penneri ATCC 35198 P_penneri-1.1.1_Cont338: NZ_ABVP01000020 | 9.1377556355866E−62 |
| 2 | NZ_ABVP01000025 | Proteus penneri ATCC 35198 P_penneri-1.1.1_Cont344: NZ_ABVP01000025 | 9.1377556355866E−62 |
| 3 | NZ_ABVP01000025 | Proteus penneri ATCC 35198 P_penneri-1.1.1_Cont344: NZ_ABVP01000025 | 9.1377556355866E−62 |
| 4 | NZ_ABVP01000023 | Proteus penneri ATCC 35198 P_penneri-1.1.1_Cont336: NZ_ABVP01000023 | 9.1377556355866E−62 |
| 5 | NZ_ABVP01000026 | Proteus penneri ATCC 35198 P_penneri-1.1.1_Cont348: NZ_ABVP01000026 | 9.1377556355866E−62 |
| 6 | NZ_ABVP01000019 | Proteus penneri ATCC 35198 P_penneri-1.1.1_Cont6.1: NZ_ABVP01000019 | 9.1377556355866E−62 |
| 7 | NZ_ABVP01000021 | Proteus penneri ATCC 35198 P_penneri-1.1.1_Cont356: NZ_ABVP01000021 | 9.1377556355866E−62 |
| 8 | NC_010554 | Proteus mirabilis HI4320: NC_010554 | 9.1377556355866E−62 |
| 9 | NZ_ABVP01000022 | Proteus penneri ATCC 35198 P_penneri-1.1.1_Cont341: NZ_ABVP01000022 | 9.1377556355866E−62 |
| 10 | NZ_ABVP01000026 | Proteus penneri ATCC 35198 P_penneri-1.1.1_Cont348: NZ_ABVP01000026 | 9.1377556355866E−62 |
| 11 | NZ_ABVP01000026 | Proteus penneri ATCC 35198 P_penneri-1.1.1_Cont348: NZ_ABVP01000026 | 9.1377556355866E−62 |
| 12 | NZ_ABVP01000014 | Proteus penneri ATCC 35198 P_penneri-1.1.1_Cont350: NZ_ABVP01000014 | 9.1377556355866E−62 |
| 13 | NZ_ABVP01000026 | Proteus penneri ATCC 35198 P_penneri-1.1.1_Cont348: NZ_ABVP01000026 | 9.1377556355866E−62 |
| 14 | NZ_ABVP01000025 | Proteus penneri ATCC 35198 P_penneri-1.1.1_Cont344: NZ_ABVP01000025 | 9.1377556355866E−62 |
| 15 | NZ_ABVP01000025 | Proteus penneri ATCC 35198 P_penneri-1.1.1_Cont344: NZ_ABVP01000025 | 9.1377556355866E−62 |
| 16 | NC_010554 | Proteus mirabilis HI4320: NC_010554 | 9.1377556355866E−62 |
| 17 | NZ_ABVP01000022 | Proteus penneri ATCC 35198 P_penneri-1.1.1_Cont341: NZ_ABVP01000022 | 9.1377556355866E−62 |
| 18 | NZ_ACLE01000064 | Proteus mirabilis ATCC 29906 contig00088: NZ_ACLE01000064 | 9.1377556355866E−62 |
| 19 | NZ_ABVP01000026 | Proteus penneri ATCC 35198 P_penneri-1.1.1_Cont348: NZ_ABVP01000026 | 9.1377556355866E−62 |
| 20 | NZ_ABVP01000026 | Proteus penneri ATCC 35198 P_penneri-1.1.1_Cont348: NZ_ABVP01000026 | 9.1377556355866E−62 |
| 21 | NZ_ABVP01000026 | Proteus penneri ATCC 35198 P_penneri-1.1.1_Cont348: NZ_ABVP01000026 | 9.1377556355866E−62 |
| 22 | NZ_ABVP01000022 | Proteus penneri ATCC 35198 P_penneri-1.1.1_Cont341: NZ_ABVP01000022 | 9.1377556355866E−62 |
| 23 | NZ_ABVP01000025 | Proteus penneri ATCC 35198 P_penneri-1.1.1_Cont344: NZ_ABVP01000025 | 9.1377556355866E−62 |
| 24 | NZ_ABVP01000025 | Proteus penneri ATCC 35198 P_penneri-1.1.1_Cont344: NZ_ABVP01000025 | 9.1377556355866E−62 |
| 25 | NZ_ABVP01000025 | Proteus penneri ATCC 35198 P_penneri-1.1.1_Cont344: NZ_ABVP01000025 | 9.1377556355866E−62 |
| 26 | NZ_ABVP01000025 | Proteus penneri ATCC 35198 P_penneri-1.1.1_Cont344: NZ_ABVP01000025 | 9.1377556355866E−62 |
| 27 | NZ_ABVP01000025 | Proteus penneri ATCC 35198 P_penneri-1.1.1_Cont344: NZ_ABVP01000025 | 9.1377556355866E−62 |
| 28 | NZ_ABVP01000025 | Proteus penneri ATCC 35198 P_penneri-1.1.1_Cont344: NZ_ABVP01000025 | 9.1377556355866E−62 |
| 29 | NC_010554 | Proteus mirabilis HI4320: NC_010554 | 9.1377556355866E−62 |
| 30 | NZ_ABVP01000021 | Proteus penneri ATCC 35198 P_penneri-1.1.1_Cont356: NZ_ABVP01000021 | 9.1377556355866E−62 |
| 31 | NZ_ABVP01000020 | Proteus penneri ATCC 35198 P_penneri-1.1.1_Cont338: NZ_ABVP01000020 | 9.1377556355866E−62 |
| 32 | NZ_ABVP01000025 | Proteus penneri ATCC 35198 P_penneri-1.1.1_Cont344: NZ_ABVP01000025 | 9.1377556355866E−62 |
| 33 | NZ_ABVP01000025 | Proteus penneri ATCC 35198 P_penneri-1.1.1_Cont344: NZ_ABVP01000025 | 9.1377556355866E−62 |
| 34 | NZ_ABVP01000023 | Proteus penneri ATCC 35198 P_penneri-1.1.1_Cont336: NZ_ABVP01000023 | 9.1377556355866E−62 |
| 35 | NZ_ABVP01000022 | Proteus penneri ATCC 35198 P_penneri-1.1.1_Cont341: NZ_ABVP01000022 | 9.1377556355866E−62 |
| 36 | NZ_ABVP01000025 | Proteus penneri ATCC 35198 P_penneri-1.1.1_Cont344: NZ_ABVP01000025 | 9.1377556355866E−62 |
| 37 | NZ_ABVP01000024 | Proteus penneri ATCC 35198 P_penneri-1.1.1_Cont334: NZ_ABVP01000024 | 9.1377556355866E−62 |
| 38 | NZ_ABVP01000025 | Proteus penneri ATCC 35198 P_penneri-1.1.1_Cont344: NZ_ABVP01000025 | 9.1377556355866E−62 |
| 39 | NC_010554 | Proteus mirabilis HI4320: NC_010554 | 9.1377556355866E−62 |
| 40 | NZ_ABVP01000019 | Proteus penneri ATCC 35198 P_penneri-1.1.1_Cont6.1: NZ_ABVP01000019 | 9.1377556355866E−62 |
| 41 | NZ_ABVP01000019 | Proteus penneri ATCC 35198 P_penneri-1.1.1_Cont6.1: NZ_ABVP01000019 | 9.1377556355866E−62 |
| 42 | NZ_ABVP01000025 | Proteus penneri ATCC 35198 P_penneri-1.1.1_Cont344: NZ_ABVP01000025 | 9.1377556355866E−62 |
| 43 | NZ_ABVP01000020 | Proteus penneri ATCC 35198 P_penneri-1.1.1_Cont338: NZ_ABVP01000020 | 9.1377556355866E−62 |
| 44 | NZ_ABVP01000023 | Proteus penneri ATCC 35198 P_penneri-1.1.1_Cont336: NZ_ABVP01000023 | 9.1377556355866E−62 |
| 45 | NZ_ABVP01000026 | Proteus penneri ATCC 35198 P_penneri-1.1.1_Cont348: NZ_ABVP01000026 | 9.1377556355866E−62 |
| 46 | NZ_ABVP01000025 | Proteus penneri ATCC 35198 P_penneri-1.1.1_Cont344: NZ_ABVP01000025 | 9.1377556355866E−62 |
| 47 | NZ_ABVP01000021 | Proteus penneri ATCC 35198 P_penneri-1.1.1_Cont356: NZ_ABVP01000021 | 9.1377556355866E−62 |
| 48 | NZ_ACLE01000079 | Proteus mirabilis ATCC 29906 contig00105: NZ_ACLE01000079 | 9.1377556355866E−62 |
| 49 | NC_010554 | Proteus mirabilis HI4320: NC_010554 | 9.1377556355866E−62 |
| 50 | NZ_ABVP01000023 | Proteus penneri ATCC 35198 P_penneri-1.1.1_Cont336: NZ_ABVP01000023 | 9.1377556355866E−62 |

TABLE 13b
Data for Proteus species (continued)

| No. | sign_phenos | sign_phenos_class | best_pheno | best_pheno_class |
|---|---|---|---|---|
| 1 | AM; CAX; CP; CRM; GM; LVX; TO; T/S | aminoglycoside; fluoroquinolone; lactam; other | CRM | lactam |
| 2 | AM; CAX; CP; CRM; GM; LVX; TO; T/S | aminoglycoside; fluoroquinolone; lactam; other | CRM | lactam |
| 3 | AM; CAX; CP; CRM; GM; LVX; TO; T/S | aminoglycoside; fluoroquinolone; lactam; other | CRM | lactam |
| 4 | AM; CAX; CP; CRM; GM; LVX; TO; T/S | aminoglycoside; fluoroquinolone; lactam; other | CRM | lactam |
| 5 | AM; CAX; CP; CRM; GM; LVX; TO; T/S | aminoglycoside; fluoroquinolone; lactam; other | CRM | lactam |
| 6 | AM; CAX; CP; CRM; GM; LVX; TO; T/S | aminoglycoside; fluoroquinolone; lactam; other | CRM | lactam |
| 7 | AM; CAX; CP; CRM; GM; LVX; TO; T/S | aminoglycoside; fluoroquinolone; lactam; other | CRM | lactam |
| 8 | AM; CAX; CP; CRM; GM; LVX; TO; T/S | aminoglycoside; fluoroquinolone; lactam; other | CRM | lactam |
| 9 | AM; CAX; CP; CRM; GM; LVX; TO; T/S | aminoglycoside; fluoroquinolone; lactam; other | CRM | lactam |
| 10 | AM; CAX; CP; CRM; GM; LVX; TO; T/S | aminoglycoside; fluoroquinolone; lactam; other | CRM | lactam |
| 11 | AM; CAX; CP; CRM; GM; LVX; TO; T/S | aminoglycoside; fluoroquinolone; lactam; other | CRM | lactam |
| 12 | AM; CAX; CP; CRM; GM; LVX; TO; T/S | aminoglycoside; fluoroquinolone; lactam; other | CRM | lactam |
| 13 | AM; CAX; CP; CRM; GM; LVX; TO; T/S | aminoglycoside; fluoroquinolone; lactam; other | CRM | lactam |
| 14 | AM; CAX; CP; CRM; GM; LVX; TO; T/S | aminoglycoside; fluoroquinolone; lactam; other | CRM | lactam |
| 15 | AM; CAX; CP; CRM; GM; LVX; TO; T/S | aminoglycoside; fluoroquinolone; lactam; other | CRM | lactam |
| 16 | AM; CAX; CP; CRM; GM; LVX; TO; T/S | aminoglycoside; fluoroquinolone; lactam; other | CRM | lactam |
| 17 | AM; CAX; CP; CRM; GM; LVX; TO; T/S | aminoglycoside; fluoroquinolone; lactam; other | CRM | lactam |
| 18 | AM; CAX; CP; CRM; GM; LVX; TO; T/S | aminoglycoside; fluoroquinolone; lactam; other | CRM | lactam |
| 19 | AM; CAX; CP; CRM; GM; LVX; TO; T/S | aminoglycoside; fluoroquinolone; lactam; other | CRM | lactam |
| 20 | AM; CAX; CP; CRM; GM; LVX; TO; T/S | aminoglycoside; fluoroquinolone; lactam; other | CRM | lactam |
| 21 | AM; CAX; CP; CRM; GM; LVX; TO; T/S | aminoglycoside; fluoroquinolone; lactam; other | CRM | lactam |
| 22 | AM; CAX; CP; CRM; GM; LVX; TO; T/S | aminoglycoside; fluoroquinolone; lactam; other | CRM | lactam |
| 23 | AM; CAX; CP; CRM; GM; LVX; TO; T/S | aminoglycoside; fluoroquinolone; lactam; other | CRM | lactam |
| 24 | AM; CAX; CP; CRM; GM; LVX; TO; T/S | aminoglycoside; fluoroquinolone; lactam; other | CRM | lactam |
| 25 | AM; CAX; CP; CRM; GM; LVX; TO; T/S | aminoglycoside; fluoroquinolone; lactam; other | CRM | lactam |
| 26 | AM; CAX; CP; CRM; GM; LVX; TO; T/S | aminoglycoside; fluoroquinolone; lactam; other | CRM | lactam |
| 27 | AM; CAX; CP; CRM; GM; LVX; TO; T/S | aminoglycoside; fluoroquinolone; lactam; other | CRM | lactam |
| 28 | AM; CAX; CP; CRM; GM; LVX; TO; T/S | aminoglycoside; fluoroquinolone; lactam; other | CRM | lactam |
| 29 | AM; CAX; CP; CRM; GM; LVX; TO; T/S | aminoglycoside; fluoroquinolone; lactam; other | CRM | lactam |
| 30 | AM; CAX; CP; CRM; GM; LVX; TO; T/S | aminoglycoside; fluoroquinolone; lactam; other | CRM | lactam |
| 31 | AM; CAX; CP; CRM; GM; LVX; TO; T/S | aminoglycoside; fluoroquinolone; lactam; other | CRM | lactam |
| 32 | AM; CAX; CP; CRM; GM; LVX; TO; T/S | aminoglycoside; fluoroquinolone; lactam; other | CRM | lactam |
| 33 | AM; CAX; CP; CRM; GM; LVX; TO; T/S | aminoglycoside; fluoroquinolone; lactam; other | CRM | lactam |
| 34 | AM; CAX; CP; CRM; GM; LVX; TO; T/S | aminoglycoside; fluoroquinolone; lactam; other | CRM | lactam |
| 35 | AM; CAX; CP; CRM; GM; LVX; TO; T/S | aminoglycoside; fluoroquinolone; lactam; other | CRM | lactam |
| 36 | AM; CAX; CP; CRM; GM; LVX; TO; T/S | aminoglycoside; fluoroquinolone; lactam; other | CRM | lactam |
| 37 | AM; CAX; CP; CRM; GM; LVX; TO; T/S | aminoglycoside; fluoroquinolone; lactam; other | CRM | lactam |
| 38 | AM; CAX; CP; CRM; GM; LVX; TO; T/S | aminoglycoside; fluoroquinolone; lactam; other | CRM | lactam |
| 39 | AM; CAX; CP; CRM; GM; LVX; TO; T/S | aminoglycoside; fluoroquinolone; lactam; other | CRM | lactam |
| 40 | AM; CAX; CP; CRM; GM; LVX; TO; T/S | aminoglycoside; fluoroquinolone; lactam; other | CRM | lactam |
| 41 | AM; CAX; CP; CRM; GM; LVX; TO; T/S | aminoglycoside; fluoroquinolone; lactam; other | CRM | lactam |
| 42 | AM; CAX; CP; CRM; GM; LVX; TO; T/S | aminoglycoside; fluoroquinolone; lactam; other | CRM | lactam |
| 43 | AM; CAX; CP; CRM; GM; LVX; TO; T/S | aminoglycoside; fluoroquinolone; lactam; other | CRM | lactam |
| 44 | AM; CAX; CP; CRM; GM; LVX; TO; T/S | aminoglycoside; fluoroquinolone; lactam; other | CRM | lactam |
| 45 | AM; CAX; CP; CRM; GM; LVX; TO; T/S | aminoglycoside; fluoroquinolone; lactam; other | CRM | lactam |
| 46 | AM; CAX; CP; CRM; GM; LVX; TO; T/S | aminoglycoside; fluoroquinolone; lactam; other | CRM | lactam |
| 47 | AM; CAX; CP; CRM; GM; LVX; TO; T/S | aminoglycoside; fluoroquinolone; lactam; other | CRM | lactam |
| 48 | AM; CAX; CP; CRM; GM; LVX; TO; T/S | aminoglycoside; fluoroquinolone; lactam; other | CRM | lactam |
| 49 | AM; CAX; CP; CRM; GM; LVX; TO; T/S | aminoglycoside; fluoroquinolone; lactam; other | CRM | lactam |
| 50 | AM; CAX; CP; CRM; GM; LVX; TO; T/S | aminoglycoside; fluoroquinolone; lactam; other | CRM | lactam |

TABLE 13c
Data for Proteus species (continued)

| No. | num_aminoglycoside | num_fluoroquinolone | num_lactam | num_other |
|---|---|---|---|---|
| 1 | 2 | 2 | 3 | 1 |
| 2 | 2 | 2 | 3 | 1 |
| 3 | 2 | 2 | 3 | 1 |
| 4 | 2 | 2 | 3 | 1 |
| 5 | 2 | 2 | 3 | 1 |
| 6 | 2 | 2 | 3 | 1 |
| 7 | 2 | 2 | 3 | 1 |
| 8 | 2 | 2 | 3 | 1 |
| 9 | 2 | 2 | 3 | 1 |
| 10 | 2 | 2 | 3 | 1 |
| 11 | 2 | 2 | 3 | 1 |
| 12 | 2 | 2 | 3 | 1 |
| 13 | 2 | 2 | 3 | 1 |
| 14 | 2 | 2 | 3 | 1 |
| 15 | 2 | 2 | 3 | 1 |
| 16 | 2 | 2 | 3 | 1 |
| 17 | 2 | 2 | 3 | 1 |
| 18 | 2 | 2 | 3 | 1 |
| 19 | 2 | 2 | 3 | 1 |
| 20 | 2 | 2 | 3 | 1 |
| 21 | 2 | 2 | 3 | 1 |
| 22 | 2 | 2 | 3 | 1 |
| 23 | 2 | 2 | 3 | 1 |
| 24 | 2 | 2 | 3 | 1 |
| 25 | 2 | 2 | 3 | 1 |
| 26 | 2 | 2 | 3 | 1 |
| 27 | 2 | 2 | 3 | 1 |
| 28 | 2 | 2 | 3 | 1 |
| 29 | 2 | 2 | 3 | 1 |
| 30 | 2 | 2 | 3 | 1 |
| 31 | 2 | 2 | 3 | 1 |
| 32 | 2 | 2 | 3 | 1 |
| 33 | 2 | 2 | 3 | 1 |
| 34 | 2 | 2 | 3 | 1 |
| 35 | 2 | 2 | 3 | 1 |
| 36 | 2 | 2 | 3 | 1 |
| 37 | 2 | 2 | 3 | 1 |
| 38 | 2 | 2 | 3 | 1 |
| 39 | 2 | 2 | 3 | 1 |
| 40 | 2 | 2 | 3 | 1 |
| 41 | 2 | 2 | 3 | 1 |
| 42 | 2 | 2 | 3 | 1 |
| 43 | 2 | 2 | 3 | 1 |
| 44 | 2 | 2 | 3 | 1 |
| 45 | 2 | 2 | 3 | 1 |
| 46 | 2 | 2 | 3 | 1 |
| 47 | 2 | 2 | 3 | 1 |
| 48 | 2 | 2 | 3 | 1 |
| 49 | 2 | 2 | 3 | 1 |
| 50 | 2 | 2 | 3 | 1 |

TABLE 13d
Data for Proteus species (continued)

| No. | AM_pv_adj | A/S_pv_adj | CAX_pv_adj | CP_pv_adj | CRM_pv_adj |
|---|---|---|---|---|---|
| 1 | 3.94E−36 | 1 | 1.38E−16 | 1.03E−08 | 9.14E−62 |
| 2 | 3.94E−36 | 1 | 1.38E−16 | 1.03E−08 | 9.14E−62 |
| 3 | 3.94E−36 | 1 | 1.38E−16 | 1.03E−08 | 9.14E−62 |
| 4 | 3.94E−36 | 1 | 1.38E−16 | 1.03E−08 | 9.14E−62 |
| 5 | 3.94E−36 | 1 | 1.38E−16 | 1.03E−08 | 9.14E−62 |
| 6 | 3.94E−36 | 1 | 1.38E−16 | 1.03E−08 | 9.14E−62 |
| 7 | 3.94E−36 | 1 | 1.38E−16 | 1.03E−08 | 9.14E−62 |
| 8 | 5.45E−37 | 9.36E−01 | 4.09E−17 | 7.21E−08 | 9.14E−62 |
| 9 | 3.94E−36 | 1 | 1.38E−16 | 1.03E−08 | 9.14E−62 |
| 10 | 3.94E−36 | 1 | 1.38E−16 | 1.03E−08 | 9.14E−62 |
| 11 | 3.94E−36 | 1 | 1.38E−16 | 1.03E−08 | 9.14E−62 |
| 12 | 3.94E−36 | 1 | 1.38E−16 | 1.03E−08 | 9.14E−62 |
| 13 | 3.94E−36 | 1 | 1.38E−16 | 1.03E−08 | 9.14E−62 |
| 14 | 3.94E−36 | 1 | 1.38E−16 | 1.03E−08 | 9.14E−62 |
| 15 | 3.94E−36 | 1 | 1.38E−16 | 1.03E−08 | 9.14E−62 |
| 16 | 3.94E−36 | 1 | 1.38E−16 | 1.03E−08 | 9.14E−62 |
| 17 | 3.94E−36 | 1 | 1.38E−16 | 1.03E−08 | 9.14E−62 |
| 18 | 3.94E−36 | 1 | 1.38E−16 | 1.03E−08 | 9.14E−62 |
| 19 | 3.94E−36 | 1 | 1.38E−16 | 1.03E−08 | 9.14E−62 |
| 20 | 3.94E−36 | 1 | 1.38E−16 | 1.03E−08 | 9.14E−62 |
| 21 | 1.07E−35 | 9.36E−01 | 8.67E−17 | 1.70E−08 | 9.14E−62 |
| 22 | 3.94E−36 | 1 | 1.38E−16 | 1.03E−08 | 9.14E−62 |
| 23 | 3.94E−36 | 1 | 1.38E−16 | 1.03E−08 | 9.14E−62 |
| 24 | 3.94E−36 | 1 | 1.38E−16 | 1.03E−08 | 9.14E−62 |
| 25 | 3.94E−36 | 1 | 1.38E−16 | 1.03E−08 | 9.14E−62 |
| 26 | 3.94E−36 | 1 | 1.38E−16 | 1.03E−08 | 9.14E−62 |
| 27 | 3.94E−36 | 1 | 1.38E−16 | 1.03E−08 | 9.14E−62 |
| 28 | 3.94E−36 | 1 | 1.38E−16 | 1.03E−08 | 9.14E−62 |
| 29 | 3.94E−36 | 1 | 1.38E−16 | 1.03E−08 | 9.14E−62 |
| 30 | 3.94E−36 | 1 | 1.38E−16 | 1.03E−08 | 9.14E−62 |
| 31 | 3.94E−36 | 1 | 1.38E−16 | 1.03E−08 | 9.14E−62 |
| 32 | 3.94E−36 | 1 | 1.38E−16 | 1.03E−08 | 9.14E−62 |
| 33 | 3.94E−36 | 1 | 1.38E−16 | 1.03E−08 | 9.14E−62 |
| 34 | 3.94E−36 | 1 | 1.38E−16 | 1.03E−08 | 9.14E−62 |
| 35 | 3.94E−36 | 1 | 1.38E−16 | 1.03E−08 | 9.14E−62 |
| 36 | 3.94E−36 | 1 | 1.38E−16 | 1.03E−08 | 9.14E−62 |
| 37 | 3.94E−36 | 1 | 1.38E−16 | 1.03E−08 | 9.14E−62 |
| 38 | 3.94E−36 | 1 | 1.38E−16 | 1.03E−08 | 9.14E−62 |
| 39 | 5.45E−37 | 9.36E−01 | 4.09E−17 | 6.06E−09 | 9.14E−62 |
| 40 | 3.94E−36 | 1 | 1.38E−16 | 1.03E−08 | 9.14E−62 |
| 41 | 3.94E−36 | 1 | 1.38E−16 | 1.03E−08 | 9.14E−62 |
| 42 | 3.94E−36 | 1 | 1.38E−16 | 1.03E−08 | 9.14E−62 |
| 43 | 3.94E−36 | 1 | 1.38E−16 | 1.03E−08 | 9.14E−62 |
| 44 | 3.94E−36 | 1 | 1.38E−16 | 1.03E−08 | 9.14E−62 |
| 45 | 3.94E−36 | 1 | 1.38E−16 | 1.03E−08 | 9.14E−62 |
| 46 | 3.94E−36 | 1 | 1.38E−16 | 1.03E−08 | 9.14E−62 |
| 47 | 1.07E−35 | 9.36E−01 | 8.67E−17 | 1.70E−08 | 9.14E−62 |
| 48 | 6.31E−38 | 5.69E−01 | 1.13E−16 | 4.44E−07 | 9.14E−62 |
| 49 | 1.56E−36 | 8.45E−01 | 2.42E−17 | 1.26E−07 | 9.14E−62 |
| 50 | 3.94E−36 | 1 | 1.38E−16 | 1.03E−08 | 9.14E−62 |

TABLE 13e
Data for Proteus species (continued)

| No. | GM_pv_adj | LVX_pv_adj | TO_pv_adj | T/S_pv_adj |
|---|---|---|---|---|
| 1 | 3.39E−06 | 1.43E−06 | 6.08E−05 | 2.82E−06 |
| 2 | 3.39E−06 | 1.43E−06 | 6.08E−05 | 2.82E−06 |
| 3 | 3.39E−06 | 1.43E−06 | 6.08E−05 | 2.82E−06 |
| 4 | 3.39E−06 | 1.43E−06 | 6.08E−05 | 2.82E−06 |
| 5 | 3.39E−06 | 1.43E−06 | 6.08E−05 | 2.82E−06 |
| 6 | 3.39E−06 | 1.43E−06 | 6.08E−05 | 2.82E−06 |
| 7 | 3.39E−06 | 1.43E−06 | 6.08E−05 | 2.82E−06 |
| 8 | 4.54E−05 | 4.64E−06 | 3.83E−05 | 1.26E−05 |
| 9 | 3.39E−06 | 1.43E−06 | 6.08E−05 | 2.82E−06 |
| 10 | 3.39E−06 | 1.43E−06 | 6.08E−05 | 2.82E−06 |
| 11 | 3.39E−06 | 1.43E−06 | 6.08E−05 | 2.82E−06 |
| 12 | 3.39E−06 | 1.43E−06 | 6.08E−05 | 2.82E−06 |
| 13 | 3.39E−06 | 1.43E−06 | 6.08E−05 | 2.82E−06 |
| 14 | 3.39E−06 | 1.43E−06 | 6.08E−05 | 2.82E−06 |
| 15 | 3.39E−06 | 1.43E−06 | 6.08E−05 | 2.82E−06 |
| 16 | 3.39E−06 | 1.43E−06 | 6.08E−05 | 2.82E−06 |
| 17 | 3.39E−06 | 1.43E−06 | 6.08E−05 | 2.82E−06 |
| 18 | 3.39E−06 | 1.43E−06 | 6.08E−05 | 2.82E−06 |
| 19 | 3.39E−06 | 1.43E−06 | 6.08E−05 | 2.82E−06 |
| 20 | 3.39E−06 | 1.43E−06 | 6.08E−05 | 2.82E−06 |
| 21 | 5.45E−06 | 1.43E−06 | 6.08E−05 | 4.40E−06 |
| 22 | 3.39E−06 | 1.43E−06 | 6.08E−05 | 2.82E−06 |
| 23 | 3.39E−06 | 1.43E−06 | 6.08E−05 | 2.82E−06 |
| 24 | 3.39E−06 | 1.43E−06 | 6.08E−05 | 2.82E−06 |
| 25 | 3.39E−06 | 1.43E−06 | 6.08E−05 | 2.82E−06 |
| 26 | 3.39E−06 | 1.43E−06 | 6.08E−05 | 2.82E−06 |
| 27 | 3.39E−06 | 1.43E−06 | 6.08E−05 | 2.82E−06 |
| 28 | 3.39E−06 | 1.43E−06 | 6.08E−05 | 2.82E−06 |
| 29 | 3.39E−06 | 1.43E−06 | 6.08E−05 | 2.82E−06 |
| 30 | 3.39E−06 | 1.43E−06 | 6.08E−05 | 2.82E−06 |
| 31 | 3.39E−06 | 1.43E−06 | 6.08E−05 | 2.82E−06 |
| 32 | 3.39E−06 | 1.43E−06 | 6.08E−05 | 2.82E−06 |
| 33 | 3.39E−06 | 1.43E−06 | 6.08E−05 | 2.82E−06 |
| 34 | 3.39E−06 | 1.43E−06 | 6.08E−05 | 2.82E−06 |
| 35 | 3.39E−06 | 1.43E−06 | 6.08E−05 | 2.82E−06 |
| 36 | 3.39E−06 | 1.43E−06 | 6.08E−05 | 2.82E−06 |
| 37 | 3.39E−06 | 1.43E−06 | 6.08E−05 | 2.82E−06 |
| 38 | 3.39E−06 | 1.43E−06 | 6.08E−05 | 2.82E−06 |
| 39 | 4.54E−05 | 8.81E−07 | 4.61E−04 | 1.26E−05 |
| 40 | 3.39E−06 | 1.43E−06 | 6.08E−05 | 2.82E−06 |
| 41 | 3.39E−06 | 1.43E−06 | 6.08E−05 | 2.82E−06 |
| 42 | 3.39E−06 | 1.43E−06 | 6.08E−05 | 2.82E−06 |
| 43 | 3.39E−06 | 1.43E−06 | 6.08E−05 | 2.82E−06 |
| 44 | 3.39E−06 | 1.43E−06 | 6.08E−05 | 2.82E−06 |
| 45 | 3.39E−06 | 1.43E−06 | 6.08E−05 | 2.82E−06 |
| 46 | 3.39E−06 | 1.43E−06 | 6.08E−05 | 2.82E−06 |
| 47 | 5.45E−06 | 1.43E−06 | 6.08E−05 | 4.40E−06 |
| 48 | 1.60E−03 | 2.08E−05 | 4.47E−04 | 7.97E−06 |
| 49 | 3.39E−06 | 7.10E−06 | 6.19E−05 | 2.82E−06 |
| 50 | 3.39E−06 | 1.43E−06 | 6.08E−05 | 2.82E−06 |

TABLE 13f
Data for Proteus species (continued)

| | | | | |
|---|---|---|---|---|
| No. | Locus.Tag | Gene.Symbol | GenBank.Accession | Chromosome |

| | | |
|---|---|---|
| 1 | PROPEN_00658 | ZP_03802316 |
| 2 | PROPEN_02821 | ZP_03804437 |
| 3 | PROPEN_02837 | ZP_03804453 |
| 4 | PROPEN_01744 | ZP_03803381 |
| 5 | PROPEN_04772 | ZP_03806369 |
| 6 | PROPEN_00566 | ZP_03802226 |
| 7 | PROPEN_00937 | ZP_03802594 |
| 8 | PMI0230 | YP_002150011 |
| 9 | PROPEN_01382 | ZP_03803029 |
| 10 | PROPEN_04308 | ZP_03805908 |
| 11 | PROPEN_03726 | ZP_03805332 |
| 12 | PROPEN_00113 | ZP_03801788 |
| 13 | PROPEN_04501 | ZP_03806101 |
| 14 | PROPEN_02952 | ZP_03804567 |
| 15 | PROPEN_03365 | ZP_03804978 |
| 16 | PMI3610 | YP_002153285 |
| 17 | PROPEN_01429 | ZP_03803076 |
| 18 | HMPREF0693_2570 | ZP_03841679 |
| 19 | PROPEN_04448 | ZP_03806048 |
| 20 | PROPEN_04169 | ZP_03805774 |
| 21 | PROPEN_04170 | ZP_03805775 |
| 22 | PROPEN_01358 | ZP_03803005 |
| 23 | PROPEN_02831 | ZP_03804447 |
| 24 | PROPEN_03253 | ZP_03804866 |
| 25 | PROPEN_03536 | ZP_03805144 |
| 26 | PROPEN_03368 | ZP_03804981 |
| 27 | PROPEN_03235 | ZP_03804848 |
| 28 | PROPEN_03484 | ZP_03805093 |
| 29 | PMI3521 | YP_002153198 |
| 30 | PROPEN_00908 | ZP_03802565 |
| 31 | PROPEN_00654 | ZP_03802312 |
| 32 | PROPEN_03299 | ZP_03804912 |
| 33 | PROPEN_03648 | ZP_03805254 |
| 34 | PROPEN_01652 | ZP_03803297 |
| 35 | PROPEN_01314 | ZP_03802961 |
| 36 | PROPEN_02647 | ZP_03804265 |
| 37 | PROPEN_02468 | ZP_03804091 |
| 38 | PROPEN_03281 | ZP_03804894 |
| 39 | PMI0234 | YP_002150015 |
| 40 | PROPEN_00544 | ZP_03802209 |
| 41 | PROPEN_00500 | ZP_03802168 |
| 42 | PROPEN_02930 | ZP_03804546 |
| 43 | PROPEN_00758 | ZP_03802416 |
| 44 | PROPEN_01810 | ZP_03803447 |
| 45 | PROPEN_04133 | ZP_03805738 |
| 46 | PROPEN_02599 | ZP_03804222 |
| 47 | PROPEN_01149 | ZP_03802800 |
| 48 | HMPREF0693_3224 | ZP_03842333 |
| 49 | PMI3023 | YP_002152722 |
| 50 | PROPEN_01623 | ZP_03803268 |

TABLE 13g
Data for Proteus species (continued)

| No. | Start.Coord | End.Coord | Strand | Scaffold.ID |
|---|---|---|---|---|
| 1 | 42080 | 42412 | + | 643889140 |
| 2 | 185454 | 186779 | − | 643889145 |
| 3 | 201544 | 202038 | + | 643889145 |
| 4 | 122639 | 124333 | − | 643889143 |
| 5 | 853119 | 853544 | + | 643889146 |
| 6 | 57621 | 58505 | − | 643889139 |
| 7 | 44265 | 45014 | + | 643889141 |
| 8 | 273673 | 274467 | + | 642555123 |
| 9 | 115768 | 116316 | − | 643889142 |
| 10 | 495434 | 496444 | − | 643889146 |
| 11 | 53270 | 53875 | − | 643889146 |
| 12 | 28708 | 29421 | − | 643889134 |
| 13 | 639163 | 640215 | − | 643889146 |
| 14 | 285467 | 286066 | − | 643889145 |
| 15 | 601755 | 602153 | − | 643889145 |
| 16 | 3948188 | 3948604 | − | 642555123 |
| 17 | 160792 | 161340 | + | 643889142 |
| 18 | 122510 | 123433 | − | 643905827 |
| 19 | 593048 | 593632 | − | 643889146 |
| 20 | 390159 | 391736 | + | 643889146 |
| 21 | 391736 | 392329 | + | 643889146 |
| 22 | 99554 | 100063 | − | 643889142 |
| 23 | 195059 | 197509 | − | 643889145 |
| 24 | 511254 | 511508 | − | 643889145 |
| 25 | 711617 | 713044 | + | 643889145 |
| 26 | 603518 | 605086 | − | 643889145 |
| 27 | 500249 | 500962 | + | 643889145 |
| 28 | 676951 | 677898 | − | 643889145 |
| 29 | 3840767 | 3841663 | − | 642555123 |
| 30 | 23566 | 24027 | − | 643889141 |
| 31 | 39429 | 39599 | + | 643889140 |
| 32 | 548639 | 549616 | − | 643889145 |
| 33 | 797158 | 797820 | + | 643889145 |
| 34 | 48570 | 49238 | − | 643889143 |
| 35 | 63033 | 63515 | + | 643889142 |
| 36 | 56690 | 57196 | − | 643889145 |
| 37 | 284519 | 287461 | + | 643889144 |
| 38 | 535246 | 536289 | + | 643889145 |
| 39 | 279605 | 281008 | + | 642555123 |
| 40 | 44077 | 44439 | − | 643889139 |
| 41 | 12698 | 14710 | − | 643889139 |
| 42 | 268387 | 269874 | − | 643889145 |
| 43 | 106241 | 106837 | − | 643889140 |
| 44 | 175312 | 176142 | + | 643889143 |
| 45 | 362809 | 363438 | + | 643889146 |
| 46 | 23652 | 24185 | + | 643889145 |
| 47 | 200043 | 201293 | − | 643889141 |
| 48 | 37235 | 38317 | − | 643905842 |
| 49 | 3320488 | 3321087 | + | 642555123 |
| 50 | 30478 | 31134 | + | 643889143 |

TABLE 14a
Data for Proteus species

| | Scaffold.External. | | |
|---|---|---|---|
| No. | Accession | Scaffold.Name | best_pv |
| 1 | NZ_ABVP01000020 | Proteus penneri ATCC 35198 P_penneri-1.1.1_Cont338: NZ_ABVP01000020 | 9.1377556355866e−62 |
| 2 | NZ_ABVP01000025 | Proteus penneri ATCC 35198 P_penneri-1.1.1_Cont344: NZ_ABVP01000025 | 9.1377556355866e−62 |
| 3 | NZ_ABVP01000025 | Proteus penneri ATCC 35198 P_penneri-1.1.1_Cont344: NZ_ABVP01000025 | 9.1377556355866e−62 |
| 4 | NZ_ABVP01000023 | Proteus penneri ATCC 35198 P_penneri-1.1.1_Cont336: NZ_ABVP01000023 | 9.1377556355866e−62 |
| 5 | NZ_ABVP01000026 | Proteus penneri ATCC 35198 P_penneri-1.1.1_Cont348: NZ_ABVP01000026 | 9.1377556355866e−62 |
| 6 | NZ_ABVP01000019 | Proteus penneri ATCC 35198 P_penneri-1.1.1_Cont6.1: NZ_ABVP01000019 | 9.1377556355866e−62 |
| 7 | NZ_ABVP01000021 | Proteus penneri ATCC 35198 P_penneri-1.1.1_Cont356: NZ_ABVP01000021 | 9.1377556355866e−62 |
| 8 | NC_010554 | Proteus mirabilis HI4320: NC_010554 | 9.1377556355866e−62 |
| 9 | NZ_ABVP01000022 | Proteus penneri ATCC 35198 P_penneri-1.1.1_Cont341: NZ_ABVP01000022 | 9.1377556355866e−62 |
| 10 | NZ_ABVP01000026 | Proteus penneri ATCC 35198 P_penneri-1.1.1_Cont348: NZ_ABVP01000026 | 9.1377556355866e−62 |
| 11 | NZ_ABVP01000026 | Proteus penneri ATCC 35198 P_penneri-1.1.1_Cont348: NZ_ABVP01000026 | 9.1377556355866e−62 |
| 12 | NZ_ABVP01000014 | Proteus penneri ATCC 35198 P_penneri-1.1.1_Cont350: NZ_ABVP01000014 | 9.1377556355866e−62 |
| 13 | NZ_ABVP01000026 | Proteus penneri ATCC 35198 P_penneri-1.1.1_Cont348: NZ_ABVP01000026 | 9.1377556355866e−62 |
| 14 | NZ_ABVP01000025 | Proteus penneri ATCC 35198 P_penneri-1.1.1_Cont344: NZ_ABVP01000025 | 9.1377556355866e−62 |
| 15 | NZ_ABVP01000025 | Proteus penneri ATCC 35198 P_penneri-1.1.1_Cont344: NZ_ABVP01000025 | 9.1377556355866e−62 |
| 16 | NC_010554 | Proteus mirabilis HI4320: NC_010554 | 9.1377556355866e−62 |
| 17 | NZ_ABVP01000022 | Proteus penneri ATCC 35198 P_penneri-1.1.1_Cont341: NZ_ABVP01000022 | 9.1377556355866e−62 |
| 18 | NZ_ACLE01000064 | Proteus mirabilis ATCC 29906 contig00088: NZ_ACLE01000064 | 9.1377556355866e−62 |
| 19 | NZ_ABVP01000026 | Proteus penneri ATCC 35198 P_penneri-1.1.1_Cont348: NZ_ABVP01000026 | 9.1377556355866e−62 |
| 20 | NZ_ABVP01000026 | Proteus penneri ATCC 35198 P_penneri-1.1.1_Cont348: NZ_ABVP01000026 | 9.1377556355866e−62 |
| 21 | NZ_ABVP01000026 | Proteus penneri ATCC 35198 P_penneri-1.1.1_Cont348: NZ_ABVP01000026 | 9.1377556355866e−62 |
| 22 | NZ_ABVP01000022 | Proteus penneri ATCC 35198 P_penneri-1.1.1_Cont341: NZ_ABVP01000022 | 9.1377556355866e−62 |
| 23 | NZ_ABVP01000025 | Proteus penneri ATCC 35198 P_penneri-1.1.1_Cont344: NZ_ABVP01000025 | 9.1377556355866e−62 |
| 24 | NZ_ABVP01000025 | Proteus penneri ATCC 35198 P_penneri-1.1.1_Cont344: NZ_ABVP01000025 | 9.1377556355866e−62 |
| 25 | NZ_ABVP01000025 | Proteus penneri ATCC 35198 P_penneri-1.1.1_Cont344: NZ_ABVP01000025 | 9.1377556355866e−62 |
| 26 | NZ_ABVP01000025 | Proteus penneri ATCC 35198 P_penneri-1.1.1_Cont344: NZ_ABVP01000025 | 9.1377556355866e−62 |
| 27 | NZ_ABVP01000025 | Proteus penneri ATCC 35198 P_penneri-1.1.1_Cont344: NZ_ABVP01000025 | 9.1377556355866e−62 |
| 28 | NZ_ABVP01000025 | Proteus penneri ATCC 35198 P_penneri-1.1.1_Cont344: NZ_ABVP01000025 | 9.1377556355866e−62 |
| 29 | NC_010554 | Proteus mirabilis HI4320: NC_010554 | 9.1377556355866e−62 |
| 30 | NZ_ABVP01000021 | Proteus penneri ATCC 35198 P_penneri-1.1.1_Cont356: NZ_ABVP01000021 | 9.1377556355866e−62 |
| 31 | NZ_ABVP01000020 | Proteus penneri ATCC 35198 P_penneri-1.1.1_Cont338: NZ_ABVP01000020 | 9.1377556355866e−62 |
| 32 | NZ_ABVP01000025 | Proteus penneri ATCC 35198 P_penneri-1.1.1_Cont344: NZ_ABVP01000025 | 9.1377556355866e−62 |
| 33 | NZ_ABVP01000025 | Proteus penneri ATCC 35198 P_penneri-1.1.1_Cont344: NZ_ABVP01000025 | 9.1377556355866e−62 |
| 34 | NZ_ABVP01000023 | Proteus penneri ATCC 35198 P_penneri-1.1.1_Cont336: NZ_ABVP01000023 | 9.1377556355866e−62 |
| 35 | NZ_ABVP01000022 | Proteus penneri ATCC 35198 P_penneri-1.1.1_Cont341: NZ_ABVP01000022 | 9.1377556355866e−62 |
| 36 | NZ_ABVP01000025 | Proteus penneri ATCC 35198 P_penneri-1.1.1_Cont344: NZ_ABVP01000025 | 9.1377556355866e−62 |
| 37 | NZ_ABVP01000024 | Proteus penneri ATCC 35198 P_penneri-1.1.1_Cont334: NZ_ABVP01000024 | 9.1377556355866e−62 |
| 38 | NZ_ABVP01000025 | Proteus penneri ATCC 35198 P_penneri-1.1.1_Cont344: NZ_ABVP01000025 | 9.1377556355866e−62 |
| 39 | NC_010554 | Proteus mirabilis HI4320: NC_010554 | 9.1377556355866e−62 |
| 40 | NZ_ABVP01000019 | Proteus penneri ATCC 35198 P_penneri-1.1.1_Cont6.1: NZ_ABVP01000019 | 9.1377556355866e−62 |
| 41 | NZ_ABVP01000019 | Proteus penneri ATCC 35198 P_penneri-1.1.1_Cont6.1: NZ_ABVP01000019 | 9.1377556355866e−62 |
| 42 | NZ_ABVP01000025 | Proteus penneri ATCC 35198 P_penneri-1.1.1_Cont344: NZ_ABVP01000025 | 9.1377556355866e−62 |
| 43 | NZ_ABVP01000020 | Proteus penneri ATCC 35198 P_penneri-1.1.1_Cont338: NZ_ABVP01000020 | 9.1377556355866e−62 |
| 44 | NZ_ABVP01000023 | Proteus penneri ATCC 35198 P_penneri-1.1.1_Cont336: NZ_ABVP01000023 | 9.1377556355866e−62 |
| 45 | NZ_ABVP01000026 | Proteus penneri ATCC 35198 P_penneri-1.1.1_Cont348: NZ_ABVP01000026 | 9.1377556355866e−62 |
| 46 | NZ_ABVP01000025 | Proteus penneri ATCC 35198 P_penneri-1.1.1_Cont344: NZ_ABVP01000025 | 9.1377556355866e−62 |
| 47 | NZ_ABVP01000021 | Proteus penneri ATCC 35198 P_penneri-1.1.1_Cont356: NZ_ABVP01000021 | 9.1377556355866e−62 |
| 48 | NZ_ACLE01000079 | Proteus mirabilis ATCC 29906 contig00105: NZ_ACLE01000079 | 9.1377556355866e−62 |
| 49 | NC_010554 | Proteus mirabilis HI4320: NC_010554 | 9.1377556355866e−62 |
| 50 | NZ_ABVP01000023 | Proteus penneri ATCC 35198 P_penneri-1.1.1_Cont336: NZ_ABVP01000023 | 9.1377556355866e−62 |

TABLE 14b
Data for Proteus species (continued)

| No. | sign_phenos | sign_phenos_class | best_pheno | best_pheno_class |
|---|---|---|---|---|
| 1 | AM; CAX; CP; CRM; GM; LVX; TO; T/S | aminoglycoside; fluoroquinolone; lactam; other | CRM | lactam |
| 2 | AM; CAX; CP; CRM; GM; LVX; TO; T/S | aminoglycoside; fluoroquinolone; lactam; other | CRM | lactam |
| 3 | AM; CAX; CP; CRM; GM; LVX; TO; T/S | aminoglycoside; fluoroquinolone; lactam; other | CRM | lactam |
| 4 | AM; CAX; CP; CRM; GM; LVX; TO; T/S | aminoglycoside; fluoroquinolone; lactam; other | CRM | lactam |
| 5 | AM; CAX; CP; CRM; GM; LVX; TO; T/S | aminoglycoside; fluoroquinolone; lactam; other | CRM | lactam |
| 6 | AM; CAX; CP; CRM; GM; LVX; TO; T/S | aminoglycoside; fluoroquinolone; lactam; other | CRM | lactam |
| 7 | AM; CAX; CP; CRM; GM; LVX; TO; T/S | aminoglycoside; fluoroquinolone; lactam; other | CRM | lactam |
| 8 | AM; CAX; CP; CRM; GM; LVX; TO; T/S | aminoglycoside; fluoroquinolone; lactam; other | CRM | lactam |
| 9 | AM; CAX; CP; CRM; GM; LVX; TO; T/S | aminoglycoside; fluoroquinolone; lactam; other | CRM | lactam |
| 10 | AM; CAX; CP; CRM; GM; LVX; TO; T/S | aminoglycoside; fluoroquinolone; lactam; other | CRM | lactam |
| 11 | AM; CAX; CP; CRM; GM; LVX; TO; T/S | aminoglycoside; fluoroquinolone; lactam; other | CRM | lactam |
| 12 | AM; CAX; CP; CRM; GM; LVX; TO; T/S | aminoglycoside; fluoroquinolone; lactam; other | CRM | lactam |
| 13 | AM; CAX; CP; CRM; GM; LVX; TO; T/S | aminoglycoside; fluoroquinolone; lactam; other | CRM | lactam |
| 14 | AM; CAX; CP; CRM; GM; LVX; TO; T/S | aminoglycoside; fluoroquinolone; lactam; other | CRM | lactam |
| 15 | AM; CAX; CP; CRM; GM; LVX; TO; T/S | aminoglycoside; fluoroquinolone; lactam; other | CRM | lactam |
| 16 | AM; CAX; CP; CRM; GM; LVX; TO; T/S | aminoglycoside; fluoroquinolone; lactam; other | CRM | lactam |
| 17 | AM; CAX; CP; CRM; GM; LVX; TO; T/S | aminoglycoside; fluoroquinolone; lactam; other | CRM | lactam |
| 18 | AM; CAX; CP; CRM; GM; LVX; TO; T/S | aminoglycoside; fluoroquinolone; lactam; other | CRM | lactam |
| 19 | AM; CAX; CP; CRM; GM; LVX; TO; T/S | aminoglycoside; fluoroquinolone; lactam; other | CRM | lactam |
| 20 | AM; CAX; CP; CRM; GM; LVX; TO; T/S | aminoglycoside; fluoroquinolone; lactam; other | CRM | lactam |
| 21 | AM; CAX; CP; CRM; GM; LVX; TO; T/S | aminoglycoside; fluoroquinolone; lactam; other | CRM | lactam |
| 22 | AM; CAX; CP; CRM; GM; LVX; TO; T/S | aminoglycoside; fluoroquinolone; lactam; other | CRM | lactam |
| 23 | AM; CAX; CP; CRM; GM; LVX; TO; T/S | aminoglycoside; fluoroquinolone; lactam; other | CRM | lactam |
| 24 | AM; CAX; CP; CRM; GM; LVX; TO; T/S | aminoglycoside; fluoroquinolone; lactam; other | CRM | lactam |
| 25 | AM; CAX; CP; CRM; GM; LVX; TO; T/S | aminoglycoside; fluoroquinolone; lactam; other | CRM | lactam |
| 26 | AM; CAX; CP; CRM; GM; LVX; TO; T/S | aminoglycoside; fluoroquinolone; lactam; other | CRM | lactam |
| 27 | AM; CAX; CP; CRM; GM; LVX; TO; T/S | aminoglycoside; fluoroquinolone; lactam; other | CRM | lactam |
| 28 | AM; CAX; CP; CRM; GM; LVX; TO; T/S | aminoglycoside; fluoroquinolone; lactam; other | CRM | lactam |
| 29 | AM; CAX; CP; CRM; GM; LVX; TO; T/S | aminoglycoside; fluoroquinolone; lactam; other | CRM | lactam |
| 30 | AM; CAX; CP; CRM; GM; LVX; TO; T/S | aminoglycoside; fluoroquinolone; lactam; other | CRM | lactam |
| 31 | AM; CAX; CP; CRM; GM; LVX; TO; T/S | aminoglycoside; fluoroquinolone; lactam; other | CRM | lactam |
| 32 | AM; CAX; CP; CRM; GM; LVX; TO; T/S | aminoglycoside; fluoroquinolone; lactam; other | CRM | lactam |
| 33 | AM; CAX; CP; CRM; GM; LVX; TO; T/S | aminoglycoside; fluoroquinolone; lactam; other | CRM | lactam |
| 34 | AM; CAX; CP; CRM; GM; LVX; TO; T/S | aminoglycoside; fluoroquinolone; lactam; other | CRM | lactam |
| 35 | AM; CAX; CP; CRM; GM; LVX; TO; T/S | aminoglycoside; fluoroquinolone; lactam; other | CRM | lactam |
| 36 | AM; CAX; CP; CRM; GM; LVX; TO; T/S | aminoglycoside; fluoroquinolone; lactam; other | CRM | lactam |
| 37 | AM; CAX; CP; CRM; GM; LVX; TO; T/S | aminoglycoside; fluoroquinolone; lactam; other | CRM | lactam |
| 38 | AM; CAX; CP; CRM; GM; LVX; TO; T/S | aminoglycoside; fluoroquinolone; lactam; other | CRM | lactam |
| 39 | AM; CAX; CP; CRM; GM; LVX; TO; T/S | aminoglycoside; fluoroquinolone; lactam; other | CRM | lactam |
| 40 | AM; CAX; CP; CRM; GM; LVX; TO; T/S | aminoglycoside; fluoroquinolone; lactam; other | CRM | lactam |
| 41 | AM; CAX; CP; CRM; GM; LVX; TO; T/S | aminoglycoside; fluoroquinolone; lactam; other | CRM | lactam |
| 42 | AM; CAX; CP; CRM; GM; LVX; TO; T/S | aminoglycoside; fluoroquinolone; lactam; other | CRM | lactam |
| 43 | AM; CAX; CP; CRM; GM; LVX; TO; T/S | aminoglycoside; fluoroquinolone; lactam; other | CRM | lactam |
| 44 | AM; CAX; CP; CRM; GM; LVX; TO; T/S | aminoglycoside; fluoroquinolone; lactam; other | CRM | lactam |
| 45 | AM; CAX; CP; CRM; GM; LVX; TO; T/S | aminoglycoside; fluoroquinolone; lactam; other | CRM | lactam |
| 46 | AM; CAX; CP; CRM; GM; LVX; TO; T/S | aminoglycoside; fluoroquinolone; lactam; other | CRM | lactam |
| 47 | AM; CAX; CP; CRM; GM; LVX; TO; T/S | aminoglycoside; fluoroquinolone; lactam; other | CRM | lactam |
| 48 | AM; CAX; CP; CRM; GM; LVX; TO; T/S | aminoglycoside; fluoroquinolone; lactam; other | CRM | lactam |
| 49 | AM; CAX; CP; CRM; GM; LVX; TO; T/S | aminoglycoside; fluoroquinolone; lactam; other | CRM | lactam |
| 50 | AM; CAX; CP; CRM; GM; LVX; TO; T/S | aminoglycoside; fluoroquinolone; lactam; other | CRM | lactam |

TABLE 14c
Data for Proteus species (continued)

| No. | num_aminoglycoside | num_fluoroquinolone | num_lactam | num_other |
|---|---|---|---|---|
| 1 | 2 | 2 | 3 | 1 |
| 2 | 2 | 2 | 3 | 1 |
| 3 | 2 | 2 | 3 | 1 |
| 4 | 2 | 2 | 3 | 1 |
| 5 | 2 | 2 | 3 | 1 |
| 6 | 2 | 2 | 3 | 1 |
| 7 | 2 | 2 | 3 | 1 |
| 8 | 2 | 2 | 3 | 1 |
| 9 | 2 | 2 | 3 | 1 |
| 10 | 2 | 2 | 3 | 1 |
| 11 | 2 | 2 | 3 | 1 |
| 12 | 2 | 2 | 3 | 1 |
| 13 | 2 | 2 | 3 | 1 |
| 14 | 2 | 2 | 3 | 1 |
| 15 | 2 | 2 | 3 | 1 |
| 16 | 2 | 2 | 3 | 1 |
| 17 | 2 | 2 | 3 | 1 |
| 18 | 2 | 2 | 3 | 1 |
| 19 | 2 | 2 | 3 | 1 |
| 20 | 2 | 2 | 3 | 1 |
| 21 | 2 | 2 | 3 | 1 |
| 22 | 2 | 2 | 3 | 1 |
| 23 | 2 | 2 | 3 | 1 |
| 24 | 2 | 2 | 3 | 1 |
| 25 | 2 | 2 | 3 | 1 |
| 26 | 2 | 2 | 3 | 1 |
| 27 | 2 | 2 | 3 | 1 |
| 28 | 2 | 2 | 3 | 1 |
| 29 | 2 | 2 | 3 | 1 |
| 30 | 2 | 2 | 3 | 1 |
| 31 | 2 | 2 | 3 | 1 |
| 32 | 2 | 2 | 3 | 1 |
| 33 | 2 | 2 | 3 | 1 |
| 34 | 2 | 2 | 3 | 1 |
| 35 | 2 | 2 | 3 | 1 |
| 36 | 2 | 2 | 3 | 1 |
| 37 | 2 | 2 | 3 | 1 |
| 38 | 2 | 2 | 3 | 1 |
| 39 | 2 | 2 | 3 | 1 |
| 40 | 2 | 2 | 3 | 1 |
| 41 | 2 | 2 | 3 | 1 |
| 42 | 2 | 2 | 3 | 1 |
| 43 | 2 | 2 | 3 | 1 |
| 44 | 2 | 2 | 3 | 1 |
| 45 | 2 | 2 | 3 | 1 |
| 46 | 2 | 2 | 3 | 1 |
| 47 | 2 | 2 | 3 | 1 |
| 48 | 2 | 2 | 3 | 1 |
| 49 | 2 | 2 | 3 | 1 |
| 50 | 2 | 2 | 3 | 1 |

TABLE 14d
Data for Proteus species (continued)

| No. | AM_pv_adj | A/S_pv_adj | CAX_pv_adj | CP_pv_adj | CRM_pv_adj |
|---|---|---|---|---|---|
| 1 | 3.94E−36 | 1 | 1.38E−16 | 1.03E−08 | 9.14E−62 |
| 2 | 3.94E−36 | 1 | 1.38E−16 | 1.03E−08 | 9.14E−62 |
| 3 | 3.94E−36 | 1 | 1.38E−16 | 1.03E−08 | 9.14E−62 |
| 4 | 3.94E−36 | 1 | 1.38E−16 | 1.03E−08 | 9.14E−62 |
| 5 | 3.94E−36 | 1 | 1.38E−16 | 1.03E−08 | 9.14E−62 |
| 6 | 3.94E−36 | 1 | 1.38E−16 | 1.03E−08 | 9.14E−62 |
| 7 | 3.94E−36 | 1 | 1.38E−16 | 1.03E−08 | 9.14E−62 |
| 8 | 5.45E−37 | 9.36E−01 | 4.09E−17 | 7.21E−08 | 9.14E−62 |
| 9 | 3.94E−36 | 1 | 1.38E−16 | 1.03E−08 | 9.14E−62 |
| 10 | 3.94E−36 | 1 | 1.38E−16 | 1.03E−08 | 9.14E−62 |
| 11 | 3.94E−36 | 1 | 1.38E−16 | 1.03E−08 | 9.14E−62 |
| 12 | 3.94E−36 | 1 | 1.38E−16 | 1.03E−08 | 9.14E−62 |
| 13 | 3.94E−36 | 1 | 1.38E−16 | 1.03E−08 | 9.14E−62 |
| 14 | 3.94E−36 | 1 | 1.38E−16 | 1.03E−08 | 9.14E−62 |
| 15 | 3.94E−36 | 1 | 1.38E−16 | 1.03E−08 | 9.14E−62 |
| 16 | 3.94E−36 | 1 | 1.38E−16 | 1.03E−08 | 9.14E−62 |
| 17 | 3.94E−36 | 1 | 1.38E−16 | 1.03E−08 | 9.14E−62 |
| 18 | 3.94E−36 | 1 | 1.38E−16 | 1.03E−08 | 9.14E−62 |
| 19 | 3.94E−36 | 1 | 1.38E−16 | 1.03E−08 | 9.14E−62 |
| 20 | 3.94E−36 | 1 | 1.38E−16 | 1.03E−08 | 9.14E−62 |
| 21 | 1.07E−35 | 9.36E−01 | 8.67E−17 | 1.70E−08 | 9.14E−62 |
| 22 | 3.94E−36 | 1 | 1.38E−16 | 1.03E−08 | 9.14E−62 |
| 23 | 3.94E−36 | 1 | 1.38E−16 | 1.03E−08 | 9.14E−62 |
| 24 | 3.94E−36 | 1 | 1.38E−16 | 1.03E−08 | 9.14E−62 |
| 25 | 3.94E−36 | 1 | 1.38E−16 | 1.03E−08 | 9.14E−62 |
| 26 | 3.94E−36 | 1 | 1.38E−16 | 1.03E−08 | 9.14E−62 |
| 27 | 3.94E−36 | 1 | 1.38E−16 | 1.03E−08 | 9.14E−62 |
| 28 | 3.94E−36 | 1 | 1.38E−16 | 1.03E−08 | 9.14E−62 |
| 29 | 3.94E−36 | 1 | 1.38E−16 | 1.03E−08 | 9.14E−62 |
| 30 | 3.94E−36 | 1 | 1.38E−16 | 1.03E−08 | 9.14E−62 |
| 31 | 3.94E−36 | 1 | 1.38E−16 | 1.03E−08 | 9.14E−62 |
| 32 | 3.94E−36 | 1 | 1.38E−16 | 1.03E−08 | 9.14E−62 |
| 33 | 3.94E−36 | 1 | 1.38E−16 | 1.03E−08 | 9.14E−62 |
| 34 | 3.94E−36 | 1 | 1.38E−16 | 1.03E−08 | 9.14E−62 |
| 35 | 3.94E−36 | 1 | 1.38E−16 | 1.03E−08 | 9.14E−62 |
| 36 | 3.94E−36 | 1 | 1.38E−16 | 1.03E−08 | 9.14E−62 |
| 37 | 3.94E−36 | 1 | 1.38E−16 | 1.03E−08 | 9.14E−62 |
| 38 | 3.94E−36 | 1 | 1.38E−16 | 1.03E−08 | 9.14E−62 |
| 39 | 5.45E−37 | 9.36E−01 | 4.09E−17 | 6.06E−09 | 9.14E−62 |
| 40 | 3.94E−36 | 1 | 1.38E−16 | 1.03E−08 | 9.14E−62 |
| 41 | 3.94E−36 | 1 | 1.38E−16 | 1.03E−08 | 9.14E−62 |
| 42 | 3.94E−36 | 1 | 1.38E−16 | 1.03E−08 | 9.14E−62 |
| 43 | 3.94E−36 | 1 | 1.38E−16 | 1.03E−08 | 9.14E−62 |
| 44 | 3.94E−36 | 1 | 1.38E−16 | 1.03E−08 | 9.14E−62 |
| 45 | 3.94E−36 | 1 | 1.38E−16 | 1.03E−08 | 9.14E−62 |
| 46 | 3.94E−36 | 1 | 1.38E−16 | 1.03E−08 | 9.14E−62 |
| 47 | 1.07E−35 | 9.36E−01 | 8.67E−17 | 1.70E−08 | 9.14E−62 |
| 48 | 6.31E−38 | 5.69E−01 | 1.13E−16 | 4.44E−07 | 9.14E−62 |
| 49 | 1.56E−36 | 8.45E−01 | 2.42E−17 | 1.26E−07 | 9.14E−62 |
| 50 | 3.94E−36 | 1 | 1.38E−16 | 1.03E−08 | 9.14E−62 |

TABLE 14e
Data for Proteus species (continued)

| No. | GM_pv_adj | LVX_pv_adj | TO_pv_adj | T/S_pv_adj |
|---|---|---|---|---|
| 1 | 3.39E−06 | 1.43E−06 | 6.08E−05 | 2.82E−06 |
| 2 | 3.39E−06 | 1.43E−06 | 6.08E−05 | 2.82E−06 |
| 3 | 3.39E−06 | 1.43E−06 | 6.08E−05 | 2.82E−06 |
| 4 | 3.39E−06 | 1.43E−06 | 6.08E−05 | 2.82E−06 |
| 5 | 3.39E−06 | 1.43E−06 | 6.08E−05 | 2.82E−06 |
| 6 | 3.39E−06 | 1.43E−06 | 6.08E−05 | 2.82E−06 |
| 7 | 3.39E−06 | 1.43E−06 | 6.08E−05 | 2.82E−06 |
| 8 | 4.54E−05 | 4.64E−06 | 3.83E−05 | 1.26E−05 |
| 9 | 3.39E−06 | 1.43E−06 | 6.08E−05 | 2.82E−06 |
| 10 | 3.39E−06 | 1.43E−06 | 6.08E−05 | 2.82E−06 |
| 11 | 3.39E−06 | 1.43E−06 | 6.08E−05 | 2.82E−06 |
| 12 | 3.39E−06 | 1.43E−06 | 6.08E−05 | 2.82E−06 |
| 13 | 3.39E−06 | 1.43E−06 | 6.08E−05 | 2.82E−06 |
| 14 | 3.39E−06 | 1.43E−06 | 6.08E−05 | 2.82E−06 |
| 15 | 3.39E−06 | 1.43E−06 | 6.08E−05 | 2.82E−06 |
| 16 | 3.39E−06 | 1.43E−06 | 6.08E−05 | 2.82E−06 |
| 17 | 3.39E−06 | 1.43E−06 | 6.08E−05 | 2.82E−06 |
| 18 | 3.39E−06 | 1.43E−06 | 6.08E−05 | 2.82E−06 |
| 19 | 3.39E−06 | 1.43E−06 | 6.08E−05 | 2.82E−06 |
| 20 | 3.39E−06 | 1.43E−06 | 6.08E−05 | 2.82E−06 |
| 21 | 5.45E−06 | 1.43E−06 | 6.08E−05 | 4.40E−06 |
| 22 | 3.39E−06 | 1.43E−06 | 6.08E−05 | 2.82E−06 |
| 23 | 3.39E−06 | 1.43E−06 | 6.08E−05 | 2.82E−06 |
| 24 | 3.39E−06 | 1.43E−06 | 6.08E−05 | 2.82E−06 |
| 25 | 3.39E−06 | 1.43E−06 | 6.08E−05 | 2.82E−06 |
| 26 | 3.39E−06 | 1.43E−06 | 6.08E−05 | 2.82E−06 |
| 27 | 3.39E−06 | 1.43E−06 | 6.08E−05 | 2.82E−06 |
| 28 | 3.39E−06 | 1.43E−06 | 6.08E−05 | 2.82E−06 |
| 29 | 3.39E−06 | 1.43E−06 | 6.08E−05 | 2.82E−06 |
| 30 | 3.39E−06 | 1.43E−06 | 6.08E−05 | 2.82E−06 |
| 31 | 3.39E−06 | 1.43E−06 | 6.08E−05 | 2.82E−06 |
| 32 | 3.39E−06 | 1.43E−06 | 6.08E−05 | 2.82E−06 |
| 33 | 3.39E−06 | 1.43E−06 | 6.08E−05 | 2.82E−06 |
| 34 | 3.39E−06 | 1.43E−06 | 6.08E−05 | 2.82E−06 |
| 35 | 3.39E−06 | 1.43E−06 | 6.08E−05 | 2.82E−06 |
| 36 | 3.39E−06 | 1.43E−06 | 6.08E−05 | 2.82E−06 |
| 37 | 3.39E−06 | 1.43E−06 | 6.08E−05 | 2.82E−06 |
| 38 | 3.39E−06 | 1.43E−06 | 6.08E−05 | 2.82E−06 |
| 39 | 4.54E−05 | 8.81E−07 | 4.61E−04 | 1.26E−05 |
| 40 | 3.39E−06 | 1.43E−06 | 6.08E−05 | 2.82E−06 |
| 41 | 3.39E−06 | 1.43E−06 | 6.08E−05 | 2.82E−06 |
| 42 | 3.39E−06 | 1.43E−06 | 6.08E−05 | 2.82E−06 |
| 43 | 3.39E−06 | 1.43E−06 | 6.08E−05 | 2.82E−06 |
| 44 | 3.39E−06 | 1.43E−06 | 6.08E−05 | 2.82E−06 |
| 45 | 3.39E−06 | 1.43E−06 | 6.08E−05 | 2.82E−06 |
| 46 | 3.39E−06 | 1.43E−06 | 6.08E−05 | 2.82E−06 |
| 47 | 5.45E−06 | 1.43E−06 | 6.08E−05 | 4.40E−06 |
| 48 | 1.60E−03 | 2.08E−05 | 4.47E−04 | 7.97E−06 |
| 49 | 3.39E−06 | 7.10E−06 | 6.19E−05 | 2.82E−06 |
| 50 | 3.39E−06 | 1.43E−06 | 6.08E−05 | 2.82E−06 |

TABLE 14f
Data for Proteus species (continued)

| | | | | |
|---|---|---|---|---|
| No. | Locus.Tag | Gene.Symbol | GenBank.Accession | Chromosome |

| | | |
|---|---|---|
| 1 | PROPEN_00658 | ZP_03802316 |
| 2 | PROPEN_02821 | ZP_03804437 |
| 3 | PROPEN_02837 | ZP_03804453 |
| 4 | PROPEN_01744 | ZP_03803381 |
| 5 | PROPEN_04772 | ZP_03806369 |
| 6 | PROPEN_00566 | ZP_03802226 |
| 7 | PROPEN_00937 | ZP_03802594 |
| 8 | PMI0230 | YP_002150011 |
| 9 | PROPEN_01382 | ZP_03803029 |
| 10 | PROPEN_04308 | ZP_03805908 |
| 11 | PROPEN_03726 | ZP_03805332 |
| 12 | PROPEN_00113 | ZP_03801788 |
| 13 | PROPEN_04501 | ZP_03806101 |
| 14 | PROPEN_02952 | ZP_03804567 |
| 15 | PROPEN_03365 | ZP_03804978 |
| 16 | PMI3610 | YP_002153285 |
| 17 | PROPEN_01429 | ZP_03803076 |
| 18 | HMPREF0693_2570 | ZP_03841679 |
| 19 | PROPEN_04448 | ZP_03806048 |
| 20 | PROPEN_04169 | ZP_03805774 |
| 21 | PROPEN_04170 | ZP_03805775 |
| 22 | PROPEN_01358 | ZP_03803005 |
| 23 | PROPEN_02831 | ZP_03804447 |
| 24 | PROPEN_03253 | ZP_03804866 |
| 25 | PROPEN_03536 | ZP_03805144 |
| 26 | PROPEN_03368 | ZP_03804981 |
| 27 | PROPEN_03235 | ZP_03804848 |
| 28 | PROPEN_03484 | ZP_03805093 |
| 29 | PMI3521 | YP_002153198 |
| 30 | PROPEN_00908 | ZP_03802565 |
| 31 | PROPEN_00654 | ZP_03802312 |
| 32 | PROPEN_03299 | ZP_03804912 |
| 33 | PROPEN_03648 | ZP_03805254 |
| 34 | PROPEN_01652 | ZP_03803297 |
| 35 | PROPEN_01314 | ZP_03802961 |
| 36 | PROPEN_02647 | ZP_03804265 |
| 37 | PROPEN_02468 | ZP_03804091 |
| 38 | PROPEN_03281 | ZP_03804894 |
| 39 | PMI0234 | YP_002150015 |
| 40 | PROPEN_00544 | ZP_03802209 |
| 41 | PROPEN_00500 | ZP_03802168 |
| 42 | PROPEN_02930 | ZP_03804546 |
| 43 | PROPEN_00758 | ZP_03802416 |
| 44 | PROPEN_01810 | ZP_03803447 |
| 45 | PROPEN_04133 | ZP_03805738 |
| 46 | PROPEN_02599 | ZP_03804222 |
| 47 | PROPEN_01149 | ZP_03802800 |
| 48 | HMPREF0693_3224 | ZP_03842333 |
| 49 | PMI3023 | YP_002152722 |
| 50 | PROPEN_01623 | ZP_03803268 |

TABLE 14g
Data for Proteus species (continued)

| No. | Start.Coord | End.Coord | Strand | Scaffold.ID |
|---|---|---|---|---|
| 1 | 42080 | 42412 | + | 643889140 |
| 2 | 185454 | 186779 | − | 643889145 |
| 3 | 201544 | 202038 | + | 643889145 |
| 4 | 122639 | 124333 | − | 643889143 |
| 5 | 853119 | 853544 | + | 643889146 |
| 6 | 57621 | 58505 | − | 643889139 |
| 7 | 44265 | 45014 | + | 643889141 |
| 8 | 273673 | 274467 | + | 642555123 |
| 9 | 115768 | 116316 | − | 643889142 |
| 10 | 495434 | 496444 | − | 643889146 |
| 11 | 53270 | 53875 | − | 643889146 |
| 12 | 28708 | 29421 | − | 643889134 |
| 13 | 639163 | 640215 | − | 643889146 |
| 14 | 285467 | 286066 | − | 643889145 |
| 15 | 601755 | 602153 | − | 643889145 |
| 16 | 3948188 | 3948604 | − | 642555123 |
| 17 | 160792 | 161340 | + | 643889142 |
| 18 | 122510 | 123433 | − | 643905827 |
| 19 | 593048 | 593632 | − | 643889146 |
| 20 | 390159 | 391736 | + | 643889146 |
| 21 | 391736 | 392329 | + | 643889146 |
| 22 | 99554 | 100063 | − | 643889142 |
| 23 | 195059 | 197509 | − | 643889145 |
| 24 | 511254 | 511508 | − | 643889145 |
| 25 | 711617 | 713044 | + | 643889145 |
| 26 | 603518 | 605086 | − | 643889145 |
| 27 | 500249 | 500962 | + | 643889145 |
| 28 | 676951 | 677898 | − | 643889145 |
| 29 | 3840767 | 3841663 | − | 642555123 |
| 30 | 23566 | 24027 | − | 643889141 |
| 31 | 39429 | 39599 | + | 643889140 |
| 32 | 548639 | 549616 | − | 643889145 |
| 33 | 797158 | 797820 | + | 643889145 |
| 34 | 48570 | 49238 | − | 643889143 |
| 35 | 63033 | 63515 | + | 643889142 |
| 36 | 56690 | 57196 | − | 643889145 |
| 37 | 284519 | 287461 | + | 643889144 |
| 38 | 535246 | 536289 | + | 643889145 |
| 39 | 279605 | 281008 | + | 642555123 |
| 40 | 44077 | 44439 | − | 643889139 |
| 41 | 12698 | 14710 | − | 643889139 |
| 42 | 268387 | 269874 | − | 643889145 |
| 43 | 106241 | 106837 | − | 643889140 |
| 44 | 175312 | 176142 | + | 643889143 |
| 45 | 362809 | 363438 | + | 643889146 |
| 46 | 23652 | 24185 | + | 643889145 |
| 47 | 200043 | 201293 | − | 643889141 |
| 48 | 37235 | 38317 | − | 643905842 |
| 49 | 3320488 | 3321087 | + | 642555123 |
| 50 | 30478 | 31134 | + | 643889143 |

### Example 8

### Pseudomonas, particularly P. aeruginosa

The procedure was carried out as in Example 1, except that the following microorganisms were used:

Bacterial Strains

The inventors selected 1042 Pseudomonas strains, particularly Pseudomonas aeruginosa, from the microbiology strain collection at Siemens Healthcare Diagnostics (West Sacramento, Calif.) for susceptibility testing and whole genome sequencing.

From MetaRef, 640 centroids of Pseudomonas aeruginosa were used as reference sequences.

The results for P. aeruginosa are shown in Tables 15 (corresponding to Table 1) and 16 (corresponding to Table 2).

TABLE 15a
Data for Pseudomonas, particularly Pseudomonas aeruginosa

| | Scaffold.External. | | |
|---|---|---|---|
| No. | Accession | Scaffold.Name | best_pv |
| 1 | NC_009656 | Pseudomonas aeruginosa PA7: NC_009656 | 4.52939243363163e−78 |
| 2 | NC_009656 | Pseudomonas aeruginosa PA7: NC_009656 | 2.75541175953672e−58 |
| 3 | NZ_AEEX01000100 | Pseudomonas aeruginosa 39016 contig00116: NZ_AEEX01000100 | 4.23483908607088e−28 |
| 4 | NC_010501 | Pseudomonas putida W619: NC_010501 | 2.9087092265635e−26 |
| 5 | NC_009656 | Pseudomonas aeruginosa PA7: NC_009656 | 5.61705273260437e−22 |
| 6 | NC_015740 | Pseudomonas stutzeri ATCC 17588 = LMG 11199 chromosome: NC_015740 | 5.61705273260437e−22 |
| 7 | NC_015733 | Pseudomonas putida S16 chromosome: NC_015733 | 1.91188334380809e−20 |
| 8 | NZ_AEEX01000148 | Pseudomonas aeruginosa 39016 contig00164: NZ_AEEX01000148 | 4.25099609715652e−20 |
| 9 | NZ_AAKV01000030 | Pseudomonas aeruginosa C3719, unfinished sequence: NZ_AAKV01000030 | 2.79764535555464e−19 |
| 10 | NZ_AEEX01000098 | Pseudomonas aeruginosa 39016 contig00114: NZ_AEEX01000098 | 4.15595600170243e−19 |
| 11 | NZ_AEEX01000163 | Pseudomonas aeruginosa 39016 contig00179: NZ_AEEX01000163 | 4.72113166087411e−19 |
| 12 | NZ_AAKW01000042 | Pseudomonas aeruginosa 2192, unfinished sequence: NZ_AAKW01000042 | 2.33370938002383e−17 |
| 13 | NZ_AEEX01000009 | Pseudomonas aeruginosa 39016 contig00011: NZ_AEEX01000009 | 3.60022886576205e−17 |
| 14 | NZ_AAKW01000075 | Pseudomonas aeruginosa 2192, unfinished sequence: NZ_AAKW01000075 | 1.13322970340506e−14 |
| 15 | NC_009656 | Pseudomonas aeruginosa PA7: NC_009656 | 2.33016029331653e−14 |
| 16 | NC_009656 | Pseudomonas aeruginosa PA7: NC_009656 | 2.33822187391836e−14 |
| 17 | NZ_AEEX01000098 | Pseudomonas aeruginosa 39016 contig00114: NZ_AEEX01000098 | 2.91419614855849e−14 |
| 18 | NC_009656 | Pseudomonas aeruginosa PA7: NC_009656 | 5.5018212149287e−14 |
| 19 | NC_010501 | Pseudomonas putida W619: NC_010501 | 6.06970436387873e−14 |
| 20 | NC_009656 | Pseudomonas aeruginosa PA7: NC_009656 | 7.75377932078369e−14 |
| 21 | NZ_AAKW01000022 | Pseudomonas aeruginosa 2192, unfinished sequence: NZ_AAKW01000022 | 3.34337634494896e−13 |
| 22 | NZ_ABKZ01000250 | Pseudomonas aeruginosa PAb1 NZ_ABKZ01000250: NZ_ABKZ01000250 | 6.17495854397665e−13 |
| 23 | NC_008463 | Pseudomonas aeruginosa UCBPP-PA14: NC_008463 | 6.42896969501816e−13 |
| 24 | NZ_AAKW01000022 | Pseudomonas aeruginosa 2192, unfinished sequence: NZ_AAKW01000022 | 1.20774339924326e−12 |
| 25 | NZ_AAQW01000001 | Pseudomonas aeruginosa PACS2, unfinished sequence: NZ_AAQW01000001 | 1.22221957191107e−12 |
| 26 | NC_011770 | Pseudomonas aeruginosa LESB58: NC_011770 | 1.79185026742242e−12 |
| 27 | NZ_AAQW01000001 | Pseudomonas aeruginosa PACS2, unfinished sequence: NZ_AAQW01000001 | 2.12378785336506e−12 |
| 28 | NZ_AAKW01000022 | Pseudomonas aeruginosa 2192, unfinished sequence: NZ_AAKW01000022 | 2.4812070369021e−12 |
| 29 | NC_009656 | Pseudomonas aeruginosa PA7: NC_009656 | 3.74499379826048e−12 |
| 30 | NZ_AEEX01000148 | Pseudomonas aeruginosa 39016 contig00164: NZ_AEEX01000148 | 4.42748938639645e−12 |
| 31 | NC_008463 | Pseudomonas aeruginosa UCBPP-PA14: NC_008463 | 5.32658560222593e−12 |
| 32 | NZ_AAQW01000001 | Pseudomonas aeruginosa PACS2, unfinished sequence: NZ_AAQW01000001 | 5.99725940121888e−12 |
| 33 | NC_009656 | Pseudomonas aeruginosa PA7: NC_009656 | 7.28057447109574e−12 |
| 34 | NC_009656 | Pseudomonas aeruginosa PA7: NC_009656 | 7.48144363663932e−12 |
| 35 | NZ_AAKW01000022 | Pseudomonas aeruginosa 2192, unfinished sequence: NZ_AAKW01000022 | 9.72376870484031e−12 |
| 36 | NZ_AEEX01000085 | Pseudomonas aeruginosa 39016 contig00101: NZ_AEEX01000085 | 9.72376870484031e−12 |
| 37 | NZ_AAQW01000001 | Pseudomonas aeruginosa PACS2, unfinished sequence: NZ_AAQW01000001 | 9.72376870484031e−12 |
| 38 | NZ_AAQW01000001 | Pseudomonas aeruginosa PACS2, unfinished sequence: NZ_AAQW01000001 | 9.72376870484031e−12 |
| 39 | NZ_AAKW01000022 | Pseudomonas aeruginosa 2192, unfinished sequence: NZ_AAKW01000022 | 9.73896479060769e−12 |
| 40 | NC_011770 | Pseudomonas aeruginosa LESB58: NC_011770 | 9.73896479060769e−12 |
| 41 | CP002622 | Pseudomonas stutzeri DSM 4166: CP002622 | 9.73896479060769e−12 |
| 42 | NZ_AAQW01000001 | Pseudomonas aeruginosa PACS2, unfinished sequence: NZ_AAQW01000001 | 9.73896479060769e−12 |
| 43 | NZ_AEEX01000149 | Pseudomonas aeruginosa 39016 contig00165: NZ_AEEX01000149 | 1.05759735249337e−11 |
| 44 | NZ_AEEX01000149 | Pseudomonas aeruginosa 39016 contig00165: NZ_AEEX01000149 | 1.05759735249337e−11 |
| 45 | NC_008463 | Pseudomonas aeruginosa UCBPP-PA14: NC_008463 | 1.05759735249337e−11 |
| 46 | NZ_AAKW01000024 | Pseudomonas aeruginosa 2192, unfinished sequence: NZ_AAKW01000024 | 1.13412272447338e−11 |
| 47 | NZ_AAKW01000022 | Pseudomonas aeruginosa 2192, unfinished sequence: NZ_AAKW01000022 | 1.64096469464127e−11 |
| 48 | NC_012560 | Azotobacter vinelandii DJ: NC_012560 | 1.85224764530357e−11 |
| 49 | NZ_AEEX01000149 | Pseudomonas aeruginosa 39016 contig00165: NZ_AEEX01000149 | 1.86265894165925e−11 |
| 50 | NC_009656 | Pseudomonas aeruginosa PA7: NC_009656 | 1.8721278417616e−11 |

TABLE 15b
Data for Pseudomonas, particularly Pseudomonas aeruginosa (continued)

| No. | sign_phenos | sign_phenos_class | best_pheno | best_pheno_class |
|---|---|---|---|---|
| 1 | AZT; CAZ; CP; CPE; GM; IMP; LVX; MER; | aminoglycoside; fluoroquinolone; lactam | TO | aminoglycoside |
| | P/T; TO | | | |
| 2 | AZT; CAZ; CP; CPE; GM; IMP; LVX; MER; | aminoglycoside; fluoroquinolone; lactam | TO | aminoglycoside |
| | P/T; TO | | | |
| 3 | CP; CPE; GM; LVX; P/T; TO | aminoglycoside; fluoroquinolone; lactam | TO | aminoglycoside |
| 4 | CP; CPE; GM; LVX; P/T; TO | aminoglycoside; fluoroquinolone; lactam | TO | aminoglycoside |
| 5 | CP; GM; LVX; TO | aminoglycoside; fluoroquinolone | TO | aminoglycoside |
| 6 | CP; GM; LVX; TO | aminoglycoside; fluoroquinolone | TO | aminoglycoside |
| 7 | CP; GM; LVX; TO | aminoglycoside; fluoroquinolone | TO | aminoglycoside |
| 8 | CP; GM; LVX; MER; P/T; TO | aminoglycoside; fluoroquinolone; lactam | TO | aminoglycoside |
| 9 | CAZ; CP; CPE; GM; LVX; MER; P/T; TO | aminoglycoside; fluoroquinolone; lactam | TO | aminoglycoside |
| 10 | CP; GM; TO | aminoglycoside; fluoroquinolone | TO | aminoglycoside |
| 11 | AZT; CAZ; CP; CPE; GM; LVX; P/T; TO | aminoglycoside; fluoroquinolone; lactam | TO | aminoglycoside |
| 12 | CAZ; CP; CPE; GM; LVX; P/T; TO | aminoglycoside; fluoroquinolone; lactam | TO | aminoglycoside |
| 13 | AZT; CAZ; CP; CPE; GM; LVX; P/T; TO | aminoglycoside; fluoroquinolone; lactam | TO | aminoglycoside |
| 14 | CP; GM; LVX; TO | aminoglycoside; fluoroquinolone | TO | aminoglycoside |
| 15 | CAZ; CP; CPE; GM; LVX; P/T; TO | aminoglycoside; fluoroquinolone; lactam | TO | aminoglycoside |
| 16 | CP; GM; IMP; LVX; MER; TO | aminoglycoside; fluoroquinolone; lactam | TO | aminoglycoside |
| 17 | CP; GM; TO | aminoglycoside; fluoroquinolone | TO | aminoglycoside |
| 18 | CAZ; CP; CPE; GM; LVX; P/T; TO | aminoglycoside; fluoroquinolone; lactam | TO | aminoglycoside |
| 19 | CP; GM; LVX; TO | aminoglycoside; fluoroquinolone | TO | aminoglycoside |
| 20 | CP; GM; LVX; TO | aminoglycoside; fluoroquinolone | TO | aminoglycoside |
| 21 | CAZ; CP; GM; MER; P/T; TO | aminoglycoside; fluoroquinolone; lactam | TO | aminoglycoside |
| 22 | CP; GM; IMP; LVX; MER; TO | aminoglycoside; fluoroquinolone; lactam | TO | aminoglycoside |
| 23 | CP; GM; TO | aminoglycoside; fluoroquinolone | TO | aminoglycoside |
| 24 | CAZ; CP; GM; MER; P/T; TO | aminoglycoside; fluoroquinolone; lactam | TO | aminoglycoside |
| 25 | CAZ; CP; GM; LVX; MER; P/T; TO | aminoglycoside; fluoroquinolone; lactam | TO | aminoglycoside |
| 26 | CAZ; CP; GM; LVX; MER; P/T; TO | aminoglycoside; fluoroquinolone; lactam | TO | aminoglycoside |
| 27 | CAZ; CP; GM; LVX; MER; P/T; TO | aminoglycoside; fluoroquinolone; lactam | TO | aminoglycoside |
| 28 | CP; GM; TO | aminoglycoside; fluoroquinolone | TO | aminoglycoside |
| 29 | CP; CPE; GM; LVX; P/T; TO | aminoglycoside; fluoroquinolone; lactam | TO | aminoglycoside |
| 30 | CP; GM; IMP; LVX; MER; TO | aminoglycoside; fluoroquinolone; lactam | TO | aminoglycoside |
| 31 | AZT; CAZ; CP; CPE; GM; LVX; P/T; TO | aminoglycoside; fluoroquinolone; lactam | TO | aminoglycoside |
| 32 | CAZ; CP; GM; LVX; MER; TO | aminoglycoside; fluoroquinolone; lactam | TO | aminoglycoside |
| 33 | CP; GM; IMP; LVX; MER; TO | aminoglycoside; fluoroquinolone; lactam | TO | aminoglycoside |
| 34 | CAZ; CP; CPE; GM; P/T; TO | aminoglycoside; fluoroquinolone; lactam | TO | aminoglycoside |
| 35 | CAZ; CP; GM; LVX; MER; P/T; TO | aminoglycoside; fluoroquinolone; lactam | TO | aminoglycoside |
| 36 | CAZ; CP; GM; LVX; MER; P/T; TO | aminoglycoside; fluoroquinolone; lactam | TO | aminoglycoside |
| 37 | CAZ; CP; GM; LVX; MER; P/T; TO | aminoglycoside; fluoroquinolone; lactam | TO | aminoglycoside |
| 38 | CAZ; CP; GM; LVX; MER; TO | aminoglycoside; fluoroquinolone; lactam | TO | aminoglycoside |
| 39 | CAZ; CP; GM; LVX; MER; P/T; TO | aminoglycoside; fluoroquinolone; lactam | TO | aminoglycoside |
| 40 | CAZ; CP; GM; LVX; MER; TO | aminoglycoside; fluoroquinolone; lactam | TO | aminoglycoside |
| 41 | CAZ; CP; GM; LVX; MER; P/T; TO | aminoglycoside; fluoroquinolone; lactam | TO | aminoglycoside |
| 42 | CAZ; CP; GM; LVX; MER; P/T; TO | aminoglycoside; fluoroquinolone; lactam | TO | aminoglycoside |
| 43 | CP; GM; IMP; LVX; TO | aminoglycoside; fluoroquinolone; lactam | TO | aminoglycoside |
| 44 | CP; GM; IMP; LVX; MER; TO | aminoglycoside; fluoroquinolone; lactam | TO | aminoglycoside |
| 45 | CP; GM; IMP; LVX; MER; TO | aminoglycoside; fluoroquinolone; lactam | TO | aminoglycoside |
| 46 | CP; GM; IMP; LVX; MER; TO | aminoglycoside; fluoroquinolone; lactam | TO | aminoglycoside |
| 47 | CAZ; CP; GM; LVX; MER; TO | aminoglycoside; fluoroquinolone; lactam | TO | aminoglycoside |
| 48 | CP; GM; IMP; LVX; MER; TO | aminoglycoside; fluoroquinolone; lactam | TO | aminoglycoside |
| 49 | CP; GM; IMP; LVX; MER; TO | aminoglycoside; fluoroquinolone; lactam | TO | aminoglycoside |
| 50 | CP; GM; IMP; LVX; TO | aminoglycoside; fluoroquinolone; lactam | TO | aminoglycoside |

TABLE 15c
Data for Pseudomonas, particularly Pseudomonas aeruginosa
(continued)

| No. | num_aminoglycoside | num_fluoroquinolone | num_lactam |
|---|---|---|---|
| 1 | 2 | 2 | 6 |
| 2 | 2 | 2 | 6 |
| 3 | 2 | 2 | 2 |
| 4 | 2 | 2 | 2 |
| 5 | 2 | 2 | 0 |
| 6 | 2 | 2 | 0 |
| 7 | 2 | 2 | 0 |
| 8 | 2 | 2 | 2 |
| 9 | 2 | 2 | 4 |
| 10 | 2 | 1 | 0 |
| 11 | 2 | 2 | 4 |
| 12 | 2 | 2 | 3 |
| 13 | 2 | 2 | 4 |
| 14 | 2 | 2 | 0 |
| 15 | 2 | 2 | 3 |
| 16 | 2 | 2 | 2 |
| 17 | 2 | 1 | 0 |
| 18 | 2 | 2 | 3 |
| 19 | 2 | 2 | 0 |
| 20 | 2 | 2 | 0 |
| 21 | 2 | 1 | 3 |
| 22 | 2 | 2 | 2 |
| 23 | 2 | 1 | 0 |
| 24 | 2 | 1 | 3 |
| 25 | 2 | 2 | 3 |
| 26 | 2 | 2 | 3 |
| 27 | 2 | 2 | 3 |
| 28 | 2 | 1 | 0 |
| 29 | 2 | 2 | 2 |
| 30 | 2 | 2 | 2 |
| 31 | 2 | 2 | 4 |
| 32 | 2 | 2 | 2 |
| 33 | 2 | 2 | 2 |
| 34 | 2 | 1 | 3 |
| 35 | 2 | 2 | 3 |
| 36 | 2 | 2 | 3 |
| 37 | 2 | 2 | 3 |
| 38 | 2 | 2 | 2 |
| 39 | 2 | 2 | 3 |
| 40 | 2 | 2 | 2 |
| 41 | 2 | 2 | 3 |
| 42 | 2 | 2 | 3 |
| 43 | 2 | 2 | 1 |
| 44 | 2 | 2 | 2 |
| 45 | 2 | 2 | 2 |
| 46 | 2 | 2 | 2 |
| 47 | 2 | 2 | 2 |
| 48 | 2 | 2 | 2 |
| 49 | 2 | 2 | 2 |
| 50 | 2 | 2 | 1 |

TABLE 15d
Data for Pseudomonas, particularly Pseudomonas aeruginosa (continued)

| No. | AZT_pv_adj | CAZ_pv_adj | CP_pv_adj | CPE_pv_adj | GM_pv_adj |
|---|---|---|---|---|---|
| 1 | 4.68E−03 | 2.31E−09 | 4.27E−32 | 5.83E−17 | 1.14E−56 |
| 2 | 1.88E−03 | 1.86E−08 | 3.37E−28 | 4.30E−16 | 2.05E−42 |
| 3 | 4.88E−01 | 1.79E−02 | 4.13E−10 | 4.41E−04 | 7.91E−18 |
| 4 | 5.97E−01 | 1.23E−02 | 2.07E−09 | 2.52E−04 | 9.24E−17 |
| 5 | 5.49E−01 | 5.49E−01 | 9.07E−06 | 1.26E−01 | 1.33E−11 |
| 6 | 5.49E−01 | 5.49E−01 | 9.07E−06 | 1.26E−01 | 1.33E−11 |
| 7 | 6.66E−01 | 3.70E−01 | 9.33E−05 | 1.41E−01 | 5.53E−11 |
| 8 | 9.33E−01 | 6.81E−02 | 1.30E−09 | 3.20E−02 | 9.74E−12 |
| 9 | 2.57E−01 | 3.98E−05 | 1.63E−09 | 1.56E−07 | 6.24E−16 |
| 10 | 7.47E−01 | 4.36E−01 | 2.70E−04 | 2.27E−01 | 5.68E−10 |
| 11 | 4.31E−03 | 4.22E−08 | 3.91E−13 | 2.77E−09 | 7.84E−16 |
| 12 | 3.06E−02 | 6.75E−05 | 1.29E−09 | 9.32E−06 | 1.77E−14 |
| 13 | 2.11E−03 | 3.40E−07 | 3.34E−10 | 7.91E−07 | 4.31E−14 |
| 14 | 6.90E−01 | 3.88E−02 | 8.36E−06 | 2.05E−02 | 1.97E−09 |
| 15 | 1.08E−01 | 8.48E−04 | 2.11E−07 | 1.99E−04 | 4.46E−13 |
| 16 | 8.07E−01 | 4.16E−02 | 3.85E−08 | 3.59E−02 | 1.19E−09 |
| 17 | 6.52E−01 | 4.40E−01 | 1.90E−04 | 5.44E−01 | 1.07E−07 |
| 18 | 1.20E−02 | 1.84E−06 | 1.64E−08 | 1.64E−07 | 4.99E−11 |
| 19 | 4.08E−01 | 1.28E−02 | 2.37E−05 | 3.15E−02 | 2.20E−09 |
| 20 | 9.48E−01 | 6.78E−02 | 9.58E−07 | 3.17E−02 | 1.32E−09 |
| 21 | 5.47E−01 | 9.24E−03 | 2.24E−04 | 5.63E−02 | 4.24E−08 |
| 22 | 8.08E−01 | 1.43E−01 | 2.42E−06 | 2.48E−01 | 1.01E−07 |
| 23 | 8.44E−01 | 2.48E−01 | 4.61E−04 | 3.25E−01 | 9.75E−07 |
| 24 | 4.14E−01 | 4.37E−03 | 1.78E−04 | 5.65E−02 | 1.49E−07 |
| 25 | 3.25E−01 | 1.51E−03 | 6.15E−05 | 2.63E−02 | 9.67E−08 |
| 26 | 6.54E−01 | 5.11E−03 | 1.38E−05 | 1.82E−02 | 5.63E−08 |
| 27 | 1.63E−01 | 1.65E−04 | 7.86E−05 | 1.59E−02 | 1.34E−09 |
| 28 | 7.69E−01 | 2.48E−01 | 7.44E−04 | 3.25E−01 | 2.80E−06 |
| 29 | 4.44E−01 | 6.54E−02 | 4.51E−05 | 8.97E−06 | 1.56E−07 |
| 30 | 9.82E−01 | 1.63E−01 | 3.28E−06 | 2.73E−01 | 2.89E−08 |
| 31 | 7.11E−03 | 2.08E−05 | 1.73E−08 | 1.31E−06 | 7.90E−09 |
| 32 | 4.78E−01 | 7.49E−03 | 4.49E−05 | 2.57E−02 | 2.96E−07 |
| 33 | 6.55E−01 | 1.81E−01 | 8.24E−06 | 2.97E−01 | 2.97E−07 |
| 34 | 5.00E−01 | 5.68E−03 | 8.53E−03 | 7.20E−03 | 1.63E−11 |
| 35 | 4.77E−01 | 4.27E−03 | 6.00E−05 | 2.17E−02 | 9.67E−08 |
| 36 | 4.77E−01 | 4.27E−03 | 6.00E−05 | 2.17E−02 | 9.67E−08 |
| 37 | 4.77E−01 | 4.27E−03 | 6.00E−05 | 2.17E−02 | 9.67E−08 |
| 38 | 5.46E−01 | 9.06E−03 | 6.00E−05 | 3.01E−02 | 2.03E−07 |
| 39 | 4.78E−01 | 4.34E−03 | 1.28E−04 | 2.20E−02 | 1.35E−07 |
| 40 | 5.47E−01 | 9.22E−03 | 7.62E−05 | 3.04E−02 | 2.83E−07 |
| 41 | 4.78E−01 | 4.34E−03 | 1.28E−04 | 2.20E−02 | 1.35E−07 |
| 42 | 4.78E−01 | 4.34E−03 | 1.28E−04 | 2.20E−02 | 1.35E−07 |
| 43 | 1 | 1.81E−01 | 8.55E−06 | 2.01E−01 | 4.71E−07 |
| 44 | 9.49E−01 | 1.43E−01 | 2.47E−06 | 2.48E−01 | 2.19E−07 |
| 45 | 1 | 1.12E−01 | 4.65E−06 | 2.01E−01 | 2.19E−07 |
| 46 | 9.82E−01 | 4.90E−02 | 6.77E−07 | 7.55E−02 | 2.07E−07 |
| 47 | 4.78E−01 | 7.49E−03 | 7.54E−05 | 2.57E−02 | 2.96E−07 |
| 48 | 9.49E−01 | 1.44E−01 | 6.20E−06 | 2.48E−01 | 4.67E−07 |
| 49 | 9.82E−01 | 1.43E−01 | 2.41E−06 | 1.62E−01 | 6.60E−07 |
| 50 | 7.30E−01 | 2.71E−01 | 1.97E−05 | 4.14E−01 | 1.33E−06 |

TABLE 15e
Data for Pseudomonas, particularly Pseudomonas aeruginosa (continued)

| No. | IMP_pv_adj | LVX_pv_adj | MER_pv_adj | P/T_pv_adj | TO_pv_adj |
|---|---|---|---|---|---|
| 1 | 1.35E−04 | 9.50E−27 | 2.29E−10 | 3.68E−18 | 4.53E−78 |
| 2 | 3.88E−03 | 4.29E−23 | 1.88E−05 | 3.33E−17 | 2.76E−58 |
| 3 | 4.33E−01 | 1.56E−06 | 1.57E−01 | 7.34E−04 | 4.23E−28 |
| 4 | 3.93E−01 | 4.01E−06 | 1.33E−01 | 9.25E−04 | 2.91E−26 |
| 5 | 2.74E−01 | 1.86E−03 | 8.61E−02 | 3.50E−01 | 5.62E−22 |
| 6 | 2.74E−01 | 1.86E−03 | 8.61E−02 | 3.50E−01 | 5.62E−22 |
| 7 | 5.97E−01 | 7.47E−03 | 1.01E−01 | 4.09E−01 | 1.91E−20 |
| 8 | 4.04E−02 | 2.96E−08 | 8.12E−03 | 3.01E−03 | 4.25E−20 |
| 9 | 4.48E−01 | 4.75E−07 | 8.35E−03 | 1.95E−05 | 2.80E−19 |
| 10 | 8.37E−01 | 1.48E−02 | 2.27E−01 | 4.37E−01 | 4.16E−19 |
| 11 | 1 | 7.77E−10 | 2.74E−02 | 2.06E−06 | 4.72E−19 |
| 12 | 8.04E−01 | 2.34E−06 | 1.05E−01 | 5.83E−04 | 2.33E−17 |
| 13 | 1 | 1.18E−06 | 1.82E−01 | 5.92E−05 | 3.60E−17 |
| 14 | 2.13E−02 | 2.85E−04 | 4.99E−01 | 1.04E−02 | 1.13E−14 |
| 15 | 4.16E−01 | 1.60E−05 | 4.41E−01 | 1.42E−03 | 2.33E−14 |
| 16 | 1.11E−03 | 7.44E−05 | 3.68E−04 | 1.33E−02 | 2.34E−14 |
| 17 | 4.55E−01 | 3.37E−02 | 3.01E−01 | 5.40E−01 | 2.91E−14 |
| 18 | 9.48E−01 | 9.19E−07 | 1.68E−01 | 5.53E−05 | 5.50E−14 |
| 19 | 1 | 1.24E−03 | 2.34E−01 | 1.09E−01 | 6.07E−14 |
| 20 | 1.05E−02 | 1.87E−04 | 2.63E−01 | 3.01E−02 | 7.75E−14 |
| 21 | 1.13E−01 | 1.21E−02 | 5.24E−03 | 6.95E−03 | 3.34E−13 |
| 22 | 2.50E−03 | 3.35E−04 | 2.39E−03 | 1.14E−01 | 6.17E−13 |
| 23 | 9.76E−02 | 4.12E−02 | 1.60E−01 | 4.20E−01 | 6.43E−13 |
| 24 | 1.75E−01 | 1.21E−02 | 8.59E−03 | 4.17E−03 | 1.21E−12 |
| 25 | 1.31E−01 | 7.32E−03 | 6.68E−03 | 1.51E−03 | 1.22E−12 |
| 26 | 9.53E−02 | 5.07E−03 | 6.50E−03 | 9.57E−03 | 1.79E−12 |
| 27 | 1.89E−02 | 8.78E−03 | 1.36E−04 | 3.10E−04 | 2.12E−12 |
| 28 | 1.14E−01 | 4.77E−02 | 1.85E−01 | 4.19E−01 | 2.48E−12 |
| 29 | 3.21E−02 | 1.60E−05 | 3.06E−01 | 2.70E−04 | 3.74E−12 |
| 30 | 4.18E−03 | 1.07E−03 | 6.72E−03 | 1.01E−01 | 4.43E−12 |
| 31 | 8.38E−01 | 7.16E−05 | 2.64E−01 | 1.39E−03 | 5.33E−12 |
| 32 | 6.86E−02 | 3.50E−03 | 3.93E−03 | 1.13E−02 | 6.00E−12 |
| 33 | 5.18E−03 | 1.07E−03 | 8.59E−03 | 1.13E−01 | 7.28E−12 |
| 34 | 9.58E−01 | 1.06E−02 | 1.47E−01 | 6.26E−03 | 7.48E−12 |
| 35 | 8.10E−02 | 4.28E−03 | 4.05E−03 | 8.08E−03 | 9.72E−12 |
| 36 | 8.10E−02 | 4.28E−03 | 4.05E−03 | 8.08E−03 | 9.72E−12 |
| 37 | 8.10E−02 | 4.28E−03 | 4.05E−03 | 8.08E−03 | 9.72E−12 |
| 38 | 8.10E−02 | 4.28E−03 | 4.05E−03 | 1.56E−02 | 9.72E−12 |
| 39 | 8.13E−02 | 7.24E−03 | 5.04E−03 | 9.57E−03 | 9.74E−12 |
| 40 | 8.13E−02 | 5.09E−03 | 5.04E−03 | 1.83E−02 | 9.74E−12 |
| 41 | 8.13E−02 | 7.24E−03 | 5.04E−03 | 9.57E−03 | 9.74E−12 |
| 42 | 8.13E−02 | 7.24E−03 | 5.04E−03 | 9.57E−03 | 9.74E−12 |
| 43 | 4.13E−03 | 1.98E−03 | 1.07E−02 | 1.29E−01 | 1.06E−11 |
| 44 | 2.49E−03 | 1.32E−03 | 4.07E−03 | 1.01E−01 | 1.06E−11 |
| 45 | 2.49E−03 | 1.98E−03 | 4.07E−03 | 7.86E−02 | 1.06E−11 |
| 46 | 6.61E−03 | 1.62E−03 | 6.66E−03 | 3.47E−02 | 1.13E−11 |
| 47 | 6.86E−02 | 5.04E−03 | 3.93E−03 | 1.56E−02 | 1.64E−11 |
| 48 | 1.92E−03 | 1.62E−03 | 5.24E−03 | 1.28E−01 | 1.85E−11 |
| 49 | 3.22E−03 | 1.08E−03 | 6.76E−03 | 7.83E−02 | 1.86E−11 |
| 50 | 6.52E−03 | 4.34E−03 | 1.32E−02 | 2.40E−01 | 1.87E−11 |

TABLE 15f
Data for Pseudomonas, particularly Pseudomonas aeruginosa (continued)

| | | | | |
|---|---|---|---|---|
| No. | Locus.Tag | Gene.Symbol | GenBank.Accession | Chromosome |

| | | | |
|---|---|---|---|
| 1 | PSPA7_5342 | | YP_001350673 |
| 2 | PSPA7_5335 | | YP_001350666 |
| 3 | PA39016_001160000 | | ZP_07794022 |
| 4 | PputW619_2322 | | YP_001749191 |
| 5 | PSPA7_0102 | | YP_001345498 |
| 6 | PSTAB_1238 | merR | YP_004713608 |
| 7 | PPS_5233 | | YP_004704640 |
| 8 | PA39016_001640030 | | ZP_07794960 |
| 9 | PaerC_01001712 | | ZP_00968897 |
| 10 | PA39016_001140033 | | ZP_07794005 |
| 11 | PA39016_001790028 | | ZP_07795388 |
| 12 | Paer2_01003738 | | ZP_00973073 |
| 13 | PA39016_000110208 | | ZP_07792174 |
| 14 | Paer2_01005870 | | ZP_00970967 |
| 15 | PSPA7_0117 | | YP_001345513 |
| 16 | PSPA7_3727 | | YP_001349081 |
| 17 | PA39016_001140034 | | ZP_07794006 |
| 18 | PSPA7_5254 | | YP_001350586 |
| 19 | PputW619_2321 | | YP_001749190 |
| 20 | PSPA7_4822 | | YP_001350163 |
| 21 | Paer2_01002125 | | ZP_00974743 |
| 22 | PaerPAb_010100012576 | | ZP_06878458 |
| 23 | PA14_15470 | merP | YP_789375 |
| 24 | Paer2_01002118 | | ZP_00974736 |
| 25 | PaerPA_01003088 | | ZP_01365958 |
| 26 | PLES_26911 | | YP_002440283 |
| 27 | PaerPA_01003092 | | ZP_01365962 |
| 28 | Paer2_01002142 | | ZP_00974760 |
| 29 | PSPA7_0357 | | YP_001345752 |
| 30 | PA39016_001640027 | | ZP_07794957 |
| 31 | PA14_49520 | pyoS3A | YP_792115 |
| 32 | PaerPA_01003114 | | ZP_01365983 |
| 33 | PSPA7_3718 | | YP_001349072 |
| 34 | PSPA7_0113 | merP1 | YP_001345509 |
| 35 | Paer2_01002136 | | ZP_00974754 |
| 36 | PA39016_001010047 | | ZP_07793693 |
| 37 | PaerPA_01003095 | | ZP_01365965 |
| 38 | PaerPA_01003132 | | ZP_01366001 |
| 39 | Paer2_01002121 | | ZP_00974739 |
| 40 | PLES_26591 | | YP_002440252 |
| 41 | PSTAA_1280 | | AEA83190 |
| 42 | PaerPA_01003094 | | ZP_01365964 |
| 43 | PA39016_001650006 | | ZP_07794967 |
| 44 | PA39016_001650001 | | ZP_07794962 |
| 45 | PA14_30900 | | YP_790630 |
| 46 | Paer2_01002387 | | ZP_00974497 |
| 47 | Paer2_01002180 | | ZP_00974798 |
| 48 | Avin_35690 | trbE | YP_002800691 |
| 49 | PA39016_001650003 | | ZP_07794964 |
| 50 | PSPA7_3698 | | YP_001349052 |

TABLE 15g
Data for Pseudomonas, particularly Pseudomonas aeruginosa (continued)

| No. | Start.Coord | End.Coord | Strand | Scaffold.ID |
|---|---|---|---|---|
| 1 | 5509888 | 5510901 | + | 640753026 |
| 2 | 5497616 | 5498380 | − | 640753026 |
| 3 | 269 | 829 | + | 649999021 |
| 4 | 2600850 | 2601410 | − | 641522670 |
| 5 | 112316 | 112732 | + | 640753026 |
| 6 | 1298354 | 1298788 | + | 650716170 |
| 7 | 5930458 | 5930823 | − | 650716164 |
| 8 | 29403 | 29642 | − | 649999069 |
| 9 | 9109 | 10122 | − | 638362473 |
| 10 | 35241 | 35606 | − | 649999019 |
| 11 | 25607 | 28270 | + | 649999084 |
| 12 | 18240 | 19688 | − | 638362609 |
| 13 | 212874 | 213821 | − | 649998930 |
| 14 | 22370 | 34666 | + | 638362642 |
| 15 | 122819 | 123664 | + | 640753026 |
| 16 | 3864574 | 3865185 | − | 640753026 |
| 17 | 35624 | 37309 | − | 649999019 |
| 18 | 5409448 | 5411001 | − | 640753026 |
| 19 | 2597880 | 2600846 | − | 641522670 |
| 20 | 4961575 | 4962753 | − | 640753026 |
| 21 | 167495 | 167935 | + | 638362589 |
| 22 | 28 | 241 | − | 647002430 |
| 23 | 1310460 | 1310735 | − | 639279323 |
| 24 | 161059 | 161934 | + | 638362589 |
| 25 | 3500513 | 3501292 | + | 638372930 |
| 26 | 2885013 | 2885837 | − | 643348637 |
| 27 | 3505406 | 3506134 | + | 638372930 |
| 28 | 182786 | 184474 | + | 638362589 |
| 29 | 366452 | 367480 | − | 640753026 |
| 30 | 26408 | 27688 | − | 649999069 |
| 31 | 4403159 | 4405534 | − | 639279323 |
| 32 | 3528459 | 3529049 | + | 638372930 |
| 33 | 3855983 | 3856195 | − | 640753026 |
| 34 | 121405 | 121680 | − | 640753026 |
| 35 | 179158 | 179895 | + | 638362589 |
| 36 | 48194 | 50227 | + | 649999006 |
| 37 | 3507452 | 3509473 | + | 638372930 |
| 38 | 3547764 | 3548102 | + | 638372930 |
| 39 | 163874 | 164434 | + | 638362589 |
| 40 | 2856655 | 2857038 | − | 643348637 |
| 41 | 1367161 | 1367601 | − | 651053081 |
| 42 | 3506732 | 3507172 | + | 638372930 |
| 43 | 6653 | 8650 | − | 649999070 |
| 44 | 2001 | 4451 | − | 649999070 |
| 45 | 2683111 | 2683836 | − | 639279323 |
| 46 | 138340 | 138654 | − | 638362591 |
| 47 | 214185 | 216386 | + | 638362589 |
| 48 | 3640230 | 3642680 | − | 643692060 |
| 49 | 4730 | 5116 | − | 649999070 |
| 50 | 3833901 | 3834893 | − | 640753026 |

TABLE 16a
Data for Pseudomonas, particularly Pseudomonas aeruginosa

| No. | Scaffold.External.Accession | Scaffold.Name | best_pv |
|---|---|---|---|
| 1 | NC_009656 | Pseudomonas aeruginosa PA7: NC_009656 | 4.52939243363163e−78 |
| 2 | NC_009656 | Pseudomonas aeruginosa PA7: NC_009656 | 2.75541175953672e−58 |
| 3 | NZ_AEEX01000100 | Pseudomonas aeruginosa 39016 contig00116: NZ_AEEX01000100 | 4.23483908607088e−28 |
| 4 | NC_010501 | Pseudomonas putida W619: NC_010501 | 2.9087092265635e−26 |
| 5 | NC_009656 | Pseudomonas aeruginosa PA7: NC_009656 | 5.61705273260437e−22 |
| 6 | NC_015740 | Pseudomonas stutzeri ATCC 17588 = LMG 11199 chromosome: NC_015740 | 5.61705273260437e−22 |
| 7 | NC_015733 | Pseudomonas putida S16 chromosome: NC_015733 | 1.91188334380809e−20 |
| 8 | NZ_AEEX01000148 | Pseudomonas aeruginosa 39016 contig00164: NZ_AEEX01000148 | 4.25099609715652e−20 |
| 9 | NZ_AAKV01000030 | Pseudomonas aeruginosa C3719, unfinished sequence: NZ_AAKV01000030 | 2.79764535555464e−19 |
| 10 | NZ_AEEX01000098 | Pseudomonas aeruginosa 39016 contig00114: NZ_AEEX01000098 | 4.15595600170243e−19 |
| 11 | NZ_AEEX01000163 | Pseudomonas aeruginosa 39016 contig00179: NZ_AEEX01000163 | 4.72113166087411e−19 |
| 12 | NZ_AAKW01000042 | Pseudomonas aeruginosa 2192, unfinished sequence: NZ_AAKW01000042 | 2.33370938002383e−17 |
| 13 | NZ_AEEX01000009 | Pseudomonas aeruginosa 39016 contig00011: NZ_AEEX01000009 | 3.60022886576205e−17 |
| 14 | NZ_AAKW01000075 | Pseudomonas aeruginosa 2192, unfinished sequence: NZ_AAKW01000075 | 1.13322970340506e−14 |
| 15 | NC_009656 | Pseudomonas aeruginosa PA7: NC_009656 | 2.33016029331653e−14 |
| 16 | NC_009656 | Pseudomonas aeruginosa PA7: NC_009656 | 2.33822187391836e−14 |
| 17 | NZ_AEEX01000098 | Pseudomonas aeruginosa 39016 contig00114: NZ_AEEX01000098 | 2.91419614855849e−14 |
| 18 | NC_009656 | Pseudomonas aeruginosa PA7: NC_009656 | 5.5018212149287e−14 |
| 19 | NC_010501 | Pseudomonas putida W619: NC_010501 | 6.06970436387873e−14 |
| 20 | NC_009656 | Pseudomonas aeruginosa PA7: NC_009656 | 7.75377932078369e−14 |
| 21 | NZ_AAKW01000022 | Pseudomonas aeruginosa 2192, unfinished sequence: NZ_AAKW01000022 | 3.34337634494896e−13 |
| 22 | NZ_ABKZ01000250 | Pseudomonas aeruginosa PAb1 NZ_ABKZ01000250: NZ_ABKZ01000250 | 6.17495854397665e−13 |
| 23 | NC_008463 | Pseudomonas aeruginosa UCBPP-PA14: NC_008463 | 6.42896969501816e−13 |
| 24 | NZ_AAKW01000022 | Pseudomonas aeruginosa 2192, unfinished sequence: NZ_AAKW01000022 | 1.20774339924326e−12 |
| 25 | NZ_AAQW01000001 | Pseudomonas aeruginosa PACS2, unfinished sequence: NZ_AAQW01000001 | 1.22221957191107e−12 |
| 26 | NC_011770 | Pseudomonas aeruginosa LESB58: NC_011770 | 1.79185026742242e−12 |
| 27 | NZ_AAQW01000001 | Pseudomonas aeruginosa PACS2, unfinished sequence: NZ_AAQW01000001 | 2.12378785336506e−12 |
| 28 | NZ_AAKW01000022 | Pseudomonas aeruginosa 2192, unfinished sequence: NZ_AAKW01000022 | 2.4812070369021e−12 |
| 29 | NC_009656 | Pseudomonas aeruginosa PA7: NC_009656 | 3.74499379826048e−12 |
| 30 | NZ_AEEX01000148 | Pseudomonas aeruginosa 39016 contig00164: NZ_AEEX01000148 | 4.42748938639645e−12 |
| 31 | NC_008463 | Pseudomonas aeruginosa UCBPP-PA14: NC_008463 | 5.32658560222593e−12 |
| 32 | NZ_AAQW01000001 | Pseudomonas aeruginosa PACS2, unfinished sequence: NZ_AAQW01000001 | 5.99725940121888e−12 |
| 33 | NC_009656 | Pseudomonas aeruginosa PA7: NC_009656 | 7.28057447109574e−12 |
| 34 | NC_009656 | Pseudomonas aeruginosa PA7: NC_009656 | 7.48144363663932e−12 |
| 35 | NZ_AAKW01000022 | Pseudomonas aeruginosa 2192, unfinished sequence: NZ_AAKW01000022 | 9.72376870484031e−12 |
| 36 | NZ_AEEX01000085 | Pseudomonas aeruginosa 39016 contig00101: NZ_AEEX01000085 | 9.72376870484031e−12 |
| 37 | NZ_AAQW01000001 | Pseudomonas aeruginosa PACS2, unfinished sequence: NZ_AAQW01000001 | 9.72376870484031e−12 |
| 38 | NZ_AAQW01000001 | Pseudomonas aeruginosa PACS2, unfinished sequence: NZ_AAQW01000001 | 9.72376870484031e−12 |
| 39 | NZ_AAKW01000022 | Pseudomonas aeruginosa 2192, unfinished sequence: NZ_AAKW01000022 | 9.73896479060769e−12 |
| 40 | NC_011770 | Pseudomonas aeruginosa LESB58: NC_011770 | 9.73896479060769e−12 |
| 41 | CP002622 | Pseudomonas stutzeri DSM 4166: CP002622 | 9.73896479060769e−12 |
| 42 | NZ_AAQW01000001 | Pseudomonas aeruginosa PACS2, unfinished sequence: NZ_AAQW01000001 | 9.73896479060769e−12 |
| 43 | NZ_AEEX01000149 | Pseudomonas aeruginosa 39016 contig00165: NZ_AEEX01000149 | 1.05759735249337e−11 |
| 44 | NZ_AEEX01000149 | Pseudomonas aeruginosa 39016 contig00165: NZ_AEEX01000149 | 1.05759735249337e−11 |
| 45 | NC_008463 | Pseudomonas aeruginosa UCBPP-PA14: NC_008463 | 1.05759735249337e−11 |
| 46 | NZ_AAKW01000024 | Pseudomonas aeruginosa 2192, unfinished sequence: NZ_AAKW01000024 | 1.13412272447338e−11 |
| 47 | NZ_AAKW01000022 | Pseudomonas aeruginosa 2192, unfinished sequence: NZ_AAKW01000022 | 1.64096469464127e−11 |
| 48 | NC_012560 | Azotobacter vinelandii DJ: NC_012560 | 1.85224764530357e−11 |
| 49 | NZ_AEEX01000149 | Pseudomonas aeruginosa 39016 contig00165: NZ_AEEX01000149 | 1.86265894165925e−11 |
| 50 | NC_009656 | Pseudomonas aeruginosa PA7: NC_009656 | 1.8721278417616e−11 |

TABLE 16b
Data for Pseudomonas, particularly Pseudomonas aeruginosa (continued)

| No. | sign_phenos | sign_phenos_class | best_pheno | best_pheno_class |
|---|---|---|---|---|
| 1 | AZT; CAZ; CP; CPE; GM; IMP; LVX; MER; | aminoglycoside; fluoroquinolone; lactam | TO | aminoglycoside |
| | P/T; TO | | | |
| 2 | AZT; CAZ; CP; CPE; GM; IMP; LVX; MER; | aminoglycoside; fluoroquinolone; lactam | TO | aminoglycoside |
| | P/T; TO | | | |
| 3 | CP; CPE; GM; LVX; P/T; TO | aminoglycoside; fluoroquinolone; lactam | TO | aminoglycoside |
| 4 | CP; CPE; GM; LVX; P/T; TO | aminoglycoside; fluoroquinolone; lactam | TO | aminoglycoside |
| 5 | CP; GM; LVX; TO | aminoglycoside; fluoroquinolone | TO | aminoglycoside |
| 6 | CP; GM; LVX; TO | aminoglycoside; fluoroquinolone | TO | aminoglycoside |
| 7 | CP; GM; LVX; TO | aminoglycoside; fluoroquinolone | TO | aminoglycoside |
| 8 | CP; GM; LVX; MER; P/T; TO | aminoglycoside; fluoroquinolone; lactam | TO | aminoglycoside |
| 9 | CAZ; CP; CPE; GM; LVX; MER; P/T; TO | aminoglycoside; fluoroquinolone; lactam | TO | aminoglycoside |
| 10 | CP; GM; TO | aminoglycoside; fluoroquinolone | TO | aminoglycoside |
| 11 | AZT; CAZ; CP; CPE; GM; LVX; P/T; TO | aminoglycoside; fluoroquinolone; lactam | TO | aminoglycoside |
| 12 | CAZ; CP; CPE; GM; LVX; P/T; TO | aminoglycoside; fluoroquinolone; lactam | TO | aminoglycoside |
| 13 | AZT; CAZ; CP; CPE; GM; LVX; P/T; TO | aminoglycoside; fluoroquinolone; lactam | TO | aminoglycoside |
| 14 | CP; GM; LVX; TO | aminoglycoside; fluoroquinolone | TO | aminoglycoside |
| 15 | CAZ; CP; CPE; GM; LVX; P/T; TO | aminoglycoside; fluoroquinolone; lactam | TO | aminoglycoside |
| 16 | CP; GM; IMP; LVX; MER; TO | aminoglycoside; fluoroquinolone; lactam | TO | aminoglycoside |
| 17 | CP; GM; TO | aminoglycoside; fluoroquinolone | TO | aminoglycoside |
| 18 | CAZ; CP; CPE; GM; LVX; P/T; TO | aminoglycoside; fluoroquinolone; lactam | TO | aminoglycoside |
| 19 | CP; GM; LVX; TO | aminoglycoside; fluoroquinolone | TO | aminoglycoside |
| 20 | CP; GM; LVX; TO | aminoglycoside; fluoroquinolone | TO | aminoglycoside |
| 21 | CAZ; CP; GM; MER; P/T; TO | aminoglycoside; fluoroquinolone; lactam | TO | aminoglycoside |
| 22 | CP; GM; IMP; LVX; MER; TO | aminoglycoside; fluoroquinolone; lactam | TO | aminoglycoside |
| 23 | CP; GM; TO | aminoglycoside; fluoroquinolone | TO | aminoglycoside |
| 24 | CAZ; CP; GM; MER; P/T; TO | aminoglycoside; fluoroquinolone; lactam | TO | aminoglycoside |
| 25 | CAZ; CP; GM; LVX; MER; P/T; TO | aminoglycoside; fluoroquinolone; lactam | TO | aminoglycoside |
| 26 | CAZ; CP; GM; LVX; MER; P/T; TO | aminoglycoside; fluoroquinolone; lactam | TO | aminoglycoside |
| 27 | CAZ; CP; GM; LVX; MER; P/T; TO | aminoglycoside; fluoroquinolone; lactam | TO | aminoglycoside |
| 28 | CP; GM; TO | aminoglycoside; fluoroquinolone | TO | aminoglycoside |
| 29 | CP; CPE; GM; LVX; P/T; TO | aminoglycoside; fluoroquinolone; lactam | TO | aminoglycoside |
| 30 | CP; GM; IMP; LVX; MER; TO | aminoglycoside; fluoroquinolone; lactam | TO | aminoglycoside |
| 31 | AZT; CAZ; CP; CPE; GM; LVX; P/T; TO | aminoglycoside; fluoroquinolone; lactam | TO | aminoglycoside |
| 32 | CAZ; CP; GM; LVX; MER; TO | aminoglycoside; fluoroquinolone; lactam | TO | aminoglycoside |
| 33 | CP; GM; IMP; LVX; MER; TO | aminoglycoside; fluoroquinolone; lactam | TO | aminoglycoside |
| 34 | CAZ; CP; CPE; GM; P/T; TO | aminoglycoside; fluoroquinolone; lactam | TO | aminoglycoside |
| 35 | CAZ; CP; GM; LVX; MER; P/T; TO | aminoglycoside; fluoroquinolone; lactam | TO | aminoglycoside |
| 36 | CAZ; CP; GM; LVX; MER; P/T; TO | aminoglycoside; fluoroquinolone; lactam | TO | aminoglycoside |
| 37 | CAZ; CP; GM; LVX; MER; P/T; TO | aminoglycoside; fluoroquinolone; lactam | TO | aminoglycoside |
| 38 | CAZ; CP; GM; LVX; MER; TO | aminoglycoside; fluoroquinolone; lactam | TO | aminoglycoside |
| 39 | CAZ; CP; GM; LVX; MER; P/T; TO | aminoglycoside; fluoroquinolone; lactam | TO | aminoglycoside |
| 40 | CAZ; CP; GM; LVX; MER; TO | aminoglycoside; fluoroquinolone; lactam | TO | aminoglycoside |
| 41 | CAZ; CP; GM; LVX; MER; P/T; TO | aminoglycoside; fluoroquinolone; lactam | TO | aminoglycoside |
| 42 | CAZ; CP; GM; LVX; MER; P/T; TO | aminoglycoside; fluoroquinolone; lactam | TO | aminoglycoside |
| 43 | CP; GM; IMP; LVX; TO | aminoglycoside; fluoroquinolone; lactam | TO | aminoglycoside |
| 44 | CP; GM; IMP; LVX; MER; TO | aminoglycoside; fluoroquinolone; lactam | TO | aminoglycoside |
| 45 | CP; GM; IMP; LVX; MER; TO | aminoglycoside; fluoroquinolone; lactam | TO | aminoglycoside |
| 46 | CP; GM; IMP; LVX; MER; TO | aminoglycoside; fluoroquinolone; lactam | TO | aminoglycoside |
| 47 | CAZ; CP; GM; LVX; MER; TO | aminoglycoside; fluoroquinolone; lactam | TO | aminoglycoside |
| 48 | CP; GM; IMP; LVX; MER; TO | aminoglycoside; fluoroquinolone; lactam | TO | aminoglycoside |
| 49 | CP; GM; IMP; LVX; MER; TO | aminoglycoside; fluoroquinolone; lactam | TO | aminoglycoside |
| 50 | CP; GM; IMP; LVX; TO | aminoglycoside; fluoroquinolone; lactam | TO | aminoglycoside |

TABLE 16c
Data for Pseudomonas, particularly Pseudomonas aeruginosa (continued)

| No. | num_aminoglycoside | num_fluoroquinolone | num_lactam |
|---|---|---|---|
| 1 | 2 | 2 | 6 |
| 2 | 2 | 2 | 6 |
| 3 | 2 | 2 | 2 |
| 4 | 2 | 2 | 2 |
| 5 | 2 | 2 | 0 |
| 6 | 2 | 2 | 0 |
| 7 | 2 | 2 | 0 |
| 8 | 2 | 2 | 2 |
| 9 | 2 | 2 | 4 |
| 10 | 2 | 1 | 0 |
| 11 | 2 | 2 | 4 |
| 12 | 2 | 2 | 3 |
| 13 | 2 | 2 | 4 |
| 14 | 2 | 2 | 0 |
| 15 | 2 | 2 | 3 |
| 16 | 2 | 2 | 2 |
| 17 | 2 | 1 | 0 |
| 18 | 2 | 2 | 3 |
| 19 | 2 | 2 | 0 |
| 20 | 2 | 2 | 0 |
| 21 | 2 | 1 | 3 |
| 22 | 2 | 2 | 2 |
| 23 | 2 | 1 | 0 |
| 24 | 2 | 1 | 3 |
| 25 | 2 | 2 | 3 |
| 26 | 2 | 2 | 3 |
| 27 | 2 | 2 | 3 |
| 28 | 2 | 1 | 0 |
| 29 | 2 | 2 | 2 |
| 30 | 2 | 2 | 2 |
| 31 | 2 | 2 | 4 |
| 32 | 2 | 2 | 2 |
| 33 | 2 | 2 | 2 |
| 34 | 2 | 1 | 3 |
| 35 | 2 | 2 | 3 |
| 36 | 2 | 2 | 3 |
| 37 | 2 | 2 | 3 |
| 38 | 2 | 2 | 2 |
| 39 | 2 | 2 | 3 |
| 40 | 2 | 2 | 2 |
| 41 | 2 | 2 | 3 |
| 42 | 2 | 2 | 3 |
| 43 | 2 | 2 | 1 |
| 44 | 2 | 2 | 2 |
| 45 | 2 | 2 | 2 |
| 46 | 2 | 2 | 2 |
| 47 | 2 | 2 | 2 |
| 48 | 2 | 2 | 2 |
| 49 | 2 | 2 | 2 |
| 50 | 2 | 2 | 1 |

TABLE 16d
Data for Pseudomonas, particularly Pseudomonas aeruginosa (continued)

| No. | AZT_pv_adj | CAZ_pv_adj | CP_pv_adj | CPE_pv_adj | GM_pv_adj |
|---|---|---|---|---|---|
| 1 | 4.68E−03 | 2.31E−09 | 4.27E−32 | 5.83E−17 | 1.14E−56 |
| 2 | 1.88E−03 | 1.86E−08 | 3.37E−28 | 4.30E−16 | 2.05E−42 |
| 3 | 4.88E−01 | 1.79E−02 | 4.13E−10 | 4.41E−04 | 7.91E−18 |
| 4 | 5.97E−01 | 1.23E−02 | 2.07E−09 | 2.52E−04 | 9.24E−17 |
| 5 | 5.49E−01 | 5.49E−01 | 9.07E−06 | 1.26E−01 | 1.33E−11 |
| 6 | 5.49E−01 | 5.49E−01 | 9.07E−06 | 1.26E−01 | 1.33E−11 |
| 7 | 6.66E−01 | 3.70E−01 | 9.33E−05 | 1.41E−01 | 5.53E−11 |
| 8 | 9.33E−01 | 6.81E−02 | 1.30E−09 | 3.20E−02 | 9.74E−12 |
| 9 | 2.57E−01 | 3.98E−05 | 1.63E−09 | 1.56E−07 | 6.24E−16 |
| 10 | 7.47E−01 | 4.36E−01 | 2.70E−04 | 2.27E−01 | 5.68E−10 |
| 11 | 4.31E−03 | 4.22E−08 | 3.91E−13 | 2.77E−09 | 7.84E−16 |
| 12 | 3.06E−02 | 6.75E−05 | 1.29E−09 | 9.32E−06 | 1.77E−14 |
| 13 | 2.11E−03 | 3.40E−07 | 3.34E−10 | 7.91E−07 | 4.31E−14 |
| 14 | 6.90E−01 | 3.88E−02 | 8.36E−06 | 2.05E−02 | 1.97E−09 |
| 15 | 1.08E−01 | 8.48E−04 | 2.11E−07 | 1.99E−04 | 4.46E−13 |
| 16 | 8.07E−01 | 4.16E−02 | 3.85E−08 | 3.59E−02 | 1.19E−09 |
| 17 | 6.52E−01 | 4.40E−01 | 1.90E−04 | 5.44E−01 | 1.07E−07 |
| 18 | 1.20E−02 | 1.84E−06 | 1.64E−08 | 1.64E−07 | 4.99E−11 |
| 19 | 4.08E−01 | 1.28E−02 | 2.37E−05 | 3.15E−02 | 2.20E−09 |
| 20 | 9.48E−01 | 6.78E−02 | 9.58E−07 | 3.17E−02 | 1.32E−09 |
| 21 | 5.47E−01 | 9.24E−03 | 2.24E−04 | 5.63E−02 | 4.24E−08 |
| 22 | 8.08E−01 | 1.43E−01 | 2.42E−06 | 2.48E−01 | 1.01E−07 |
| 23 | 8.44E−01 | 2.48E−01 | 4.61E−04 | 3.25E−01 | 9.75E−07 |
| 24 | 4.14E−01 | 4.37E−03 | 1.78E−04 | 5.65E−02 | 1.49E−07 |
| 25 | 3.25E−01 | 1.51E−03 | 6.15E−05 | 2.63E−02 | 9.67E−08 |
| 26 | 6.54E−01 | 5.11E−03 | 1.38E−05 | 1.82E−02 | 5.63E−08 |
| 27 | 1.63E−01 | 1.65E−04 | 7.86E−05 | 1.59E−02 | 1.34E−09 |
| 28 | 7.69E−01 | 2.48E−01 | 7.44E−04 | 3.25E−01 | 2.80E−06 |
| 29 | 4.44E−01 | 6.54E−02 | 4.51E−05 | 8.97E−06 | 1.56E−07 |
| 30 | 9.82E−01 | 1.63E−01 | 3.28E−06 | 2.73E−01 | 2.89E−08 |
| 31 | 7.11E−03 | 2.08E−05 | 1.73E−08 | 1.31E−06 | 7.90E−09 |
| 32 | 4.78E−01 | 7.49E−03 | 4.49E−05 | 2.57E−02 | 2.96E−07 |
| 33 | 6.55E−01 | 1.81E−01 | 8.24E−06 | 2.97E−01 | 2.97E−07 |
| 34 | 5.00E−01 | 5.68E−03 | 8.53E−03 | 7.20E−03 | 1.63E−11 |
| 35 | 4.77E−01 | 4.27E−03 | 6.00E−05 | 2.17E−02 | 9.67E−08 |
| 36 | 4.77E−01 | 4.27E−03 | 6.00E−05 | 2.17E−02 | 9.67E−08 |
| 37 | 4.77E−01 | 4.27E−03 | 6.00E−05 | 2.17E−02 | 9.67E−08 |
| 38 | 5.46E−01 | 9.06E−03 | 6.00E−05 | 3.01E−02 | 2.03E−07 |
| 39 | 4.78E−01 | 4.34E−03 | 1.28E−04 | 2.20E−02 | 1.35E−07 |
| 40 | 5.47E−01 | 9.22E−03 | 7.62E−05 | 3.04E−02 | 2.83E−07 |
| 41 | 4.78E−01 | 4.34E−03 | 1.28E−04 | 2.20E−02 | 1.35E−07 |
| 42 | 4.78E−01 | 4.34E−03 | 1.28E−04 | 2.20E−02 | 1.35E−07 |
| 43 | 1 | 1.81E−01 | 8.55E−06 | 2.01E−01 | 4.71E−07 |
| 44 | 9.49E−01 | 1.43E−01 | 2.47E−06 | 2.48E−01 | 2.19E−07 |
| 45 | 1 | 1.12E−01 | 4.65E−06 | 2.01E−01 | 2.19E−07 |
| 46 | 9.82E−01 | 4.90E−02 | 6.77E−07 | 7.55E−02 | 2.07E−07 |
| 47 | 4.78E−01 | 7.49E−03 | 7.54E−05 | 2.57E−02 | 2.96E−07 |
| 48 | 9.49E−01 | 1.44E−01 | 6.20E−06 | 2.48E−01 | 4.67E−07 |
| 49 | 9.82E−01 | 1.43E−01 | 2.41E−06 | 1.62E−01 | 6.60E−07 |
| 50 | 7.30E−01 | 2.71E−01 | 1.97E−05 | 4.14E−01 | 1.33E−06 |

TABLE 16e
Data for Pseudomonas, particularly Pseudomonas aeruginosa (continued)

| No. | IMP_pv_adj | LVX_pv_adj | MER_pv_adj | P/T_pv_adj | TO_pv_adj |
|---|---|---|---|---|---|
| 1 | 1.35E−04 | 9.50E−27 | 2.29E−10 | 3.68E−18 | 4.53E−78 |
| 2 | 3.88E−03 | 4.29E−23 | 1.88E−05 | 3.33E−17 | 2.76E−58 |
| 3 | 4.33E−01 | 1.56E−06 | 1.57E−01 | 7.34E−04 | 4.23E−28 |
| 4 | 3.93E−01 | 4.01E−06 | 1.33E−01 | 9.25E−04 | 2.91E−26 |
| 5 | 2.74E−01 | 1.86E−03 | 8.61E−02 | 3.50E−01 | 5.62E−22 |
| 6 | 2.74E−01 | 1.86E−03 | 8.61E−02 | 3.50E−01 | 5.62E−22 |
| 7 | 5.97E−01 | 7.47E−03 | 1.01E−01 | 4.09E−01 | 1.91E−20 |
| 8 | 4.04E−02 | 2.96E−08 | 8.12E−03 | 3.01E−03 | 4.25E−20 |
| 9 | 4.48E−01 | 4.75E−07 | 8.35E−03 | 1.95E−05 | 2.80E−19 |
| 10 | 8.37E−01 | 1.48E−02 | 2.27E−01 | 4.37E−01 | 4.16E−19 |
| 11 | 1 | 7.77E−10 | 2.74E−02 | 2.06E−06 | 4.72E−19 |
| 12 | 8.04E−01 | 2.34E−06 | 1.05E−01 | 5.83E−04 | 2.33E−17 |
| 13 | 1 | 1.18E−06 | 1.82E−01 | 5.92E−05 | 3.60E−17 |
| 14 | 2.13E−02 | 2.85E−04 | 4.99E−01 | 1.04E−02 | 1.13E−14 |
| 15 | 4.16E−01 | 1.60E−05 | 4.41E−01 | 1.42E−03 | 2.33E−14 |
| 16 | 1.11E−03 | 7.44E−05 | 3.68E−04 | 1.33E−02 | 2.34E−14 |
| 17 | 4.55E−01 | 3.37E−02 | 3.01E−01 | 5.40E−01 | 2.91E−14 |
| 18 | 9.48E−01 | 9.19E−07 | 1.68E−01 | 5.53E−05 | 5.50E−14 |
| 19 | 1 | 1.24E−03 | 2.34E−01 | 1.09E−01 | 6.07E−14 |
| 20 | 1.05E−02 | 1.87E−04 | 2.63E−01 | 3.01E−02 | 7.75E−14 |
| 21 | 1.13E−01 | 1.21E−02 | 5.24E−03 | 6.95E−03 | 3.34E−13 |
| 22 | 2.50E−03 | 3.35E−04 | 2.39E−03 | 1.14E−01 | 6.17E−13 |
| 23 | 9.76E−02 | 4.12E−02 | 1.60E−01 | 4.20E−01 | 6.43E−13 |
| 24 | 1.75E−01 | 1.21E−02 | 8.59E−03 | 4.17E−03 | 1.21E−12 |
| 25 | 1.31E−01 | 7.32E−03 | 6.68E−03 | 1.51E−03 | 1.22E−12 |
| 26 | 9.53E−02 | 5.07E−03 | 6.50E−03 | 9.57E−03 | 1.79E−12 |
| 27 | 1.89E−02 | 8.78E−03 | 1.36E−04 | 3.10E−04 | 2.12E−12 |
| 28 | 1.14E−01 | 4.77E−02 | 1.85E−01 | 4.19E−01 | 2.48E−12 |
| 29 | 3.21E−02 | 1.60E−05 | 3.06E−01 | 2.70E−04 | 3.74E−12 |
| 30 | 4.18E−03 | 1.07E−03 | 6.72E−03 | 1.01E−01 | 4.43E−12 |
| 31 | 8.38E−01 | 7.16E−05 | 2.64E−01 | 1.39E−03 | 5.33E−12 |
| 32 | 6.86E−02 | 3.50E−03 | 3.93E−03 | 1.13E−02 | 6.00E−12 |
| 33 | 5.18E−03 | 1.07E−03 | 8.59E−03 | 1.13E−01 | 7.28E−12 |
| 34 | 9.58E−01 | 1.06E−02 | 1.47E−01 | 6.26E−03 | 7.48E−12 |
| 35 | 8.10E−02 | 4.28E−03 | 4.05E−03 | 8.08E−03 | 9.72E−12 |
| 36 | 8.10E−02 | 4.28E−03 | 4.05E−03 | 8.08E−03 | 9.72E−12 |
| 37 | 8.10E−02 | 4.28E−03 | 4.05E−03 | 8.08E−03 | 9.72E−12 |
| 38 | 8.10E−02 | 4.28E−03 | 4.05E−03 | 1.56E−02 | 9.72E−12 |
| 39 | 8.13E−02 | 7.24E−03 | 5.04E−03 | 9.57E−03 | 9.74E−12 |
| 40 | 8.13E−02 | 5.09E−03 | 5.04E−03 | 1.83E−02 | 9.74E−12 |
| 41 | 8.13E−02 | 7.24E−03 | 5.04E−03 | 9.57E−03 | 9.74E−12 |
| 42 | 8.13E−02 | 7.24E−03 | 5.04E−03 | 9.57E−03 | 9.74E−12 |
| 43 | 4.13E−03 | 1.98E−03 | 1.07E−02 | 1.29E−01 | 1.06E−11 |
| 44 | 2.49E−03 | 1.32E−03 | 4.07E−03 | 1.01E−01 | 1.06E−11 |
| 45 | 2.49E−03 | 1.98E−03 | 4.07E−03 | 7.86E−02 | 1.06E−11 |
| 46 | 6.61E−03 | 1.62E−03 | 6.66E−03 | 3.47E−02 | 1.13E−11 |
| 47 | 6.86E−02 | 5.04E−03 | 3.93E−03 | 1.56E−02 | 1.64E−11 |
| 48 | 1.92E−03 | 1.62E−03 | 5.24E−03 | 1.28E−01 | 1.85E−11 |
| 49 | 3.22E−03 | 1.08E−03 | 6.76E−03 | 7.83E−02 | 1.86E−11 |
| 50 | 6.52E−03 | 4.34E−03 | 1.32E−02 | 2.40E−01 | 1.87E−11 |

TABLE 16f
Data for Pseudomonas, particularly Pseudomonas aeruginosa (continued)

| | | | | |
|---|---|---|---|---|
| No. | Locus.Tag | Gene.Symbol | GenBank.Accession | Chromosome |

| | | | |
|---|---|---|---|
| 1 | PSPA7_5342 | | YP_001350673 |
| 2 | PSPA7_5335 | | YP_001350666 |
| 3 | PA39016_001160000 | | ZP_07794022 |
| 4 | PputW619_2322 | | YP_001749191 |
| 5 | PSPA7_0102 | | YP_001345498 |
| 6 | PSTAB_1238 | merR | YP_004713608 |
| 7 | PPS_5233 | | YP_004704640 |
| 8 | PA39016_001640030 | | ZP_07794960 |
| 9 | PaerC_01001712 | | ZP_00968897 |
| 10 | PA39016_001140033 | | ZP_07794005 |
| 11 | PA39016_001790028 | | ZP_07795388 |
| 12 | Paer2_01003738 | | ZP_00973073 |
| 13 | PA39016_000110208 | | ZP_07792174 |
| 14 | Paer2_01005870 | | ZP_00970967 |
| 15 | PSPA7_0117 | | YP_001345513 |
| 16 | PSPA7_3727 | | YP_001349081 |
| 17 | PA39016_001140034 | | ZP_07794006 |
| 18 | PSPA7_5254 | | YP_001350586 |
| 19 | PputW619_2321 | | YP_001749190 |
| 20 | PSPA7_4822 | | YP_001350163 |
| 21 | Paer2_01002125 | | ZP_00974743 |
| 22 | PaerPAb_010100012576 | | ZP_06878458 |
| 23 | PA14_15470 | merP | YP_789375 |
| 24 | Paer2_01002118 | | ZP_00974736 |
| 25 | PaerPA_01003088 | | ZP_01365958 |
| 26 | PLES_26911 | | YP_002440283 |
| 27 | PaerPA_01003092 | | ZP_01365962 |
| 28 | Paer2_01002142 | | ZP_00974760 |
| 29 | PSPA7_0357 | | YP_001345752 |
| 30 | PA39016_001640027 | | ZP_07794957 |
| 31 | PA14_49520 | pyoS3A | YP_792115 |
| 32 | PaerPA_01003114 | | ZP_01365983 |
| 33 | PSPA7_3718 | | YP_001349072 |
| 34 | PSPA7_0113 | merP1 | YP_001345509 |
| 35 | Paer2_01002136 | | ZP_00974754 |
| 36 | PA39016_001010047 | | ZP_07793693 |
| 37 | PaerPA_01003095 | | ZP_01365965 |
| 38 | PaerPA_01003132 | | ZP_01366001 |
| 39 | Paer2_01002121 | | ZP_00974739 |
| 40 | PLES_26591 | | YP_002440252 |
| 41 | PSTAA_1280 | | AEA83190 |
| 42 | PaerPA_01003094 | | ZP_01365964 |
| 43 | PA39016_001650006 | | ZP_07794967 |
| 44 | PA39016_001650001 | | ZP_07794962 |
| 45 | PA14_30900 | | YP_790630 |
| 46 | Paer2_01002387 | | ZP_00974497 |
| 47 | Paer2_01002180 | | ZP_00974798 |
| 48 | Avin_35690 | trbE | YP_002800691 |
| 49 | PA39016_001650003 | | ZP_07794964 |
| 50 | PSPA7_3698 | | YP_001349052 |

TABLE 16g
Data for Pseudomonas, particularly
Pseudomonas aeruginosa (continued)

| No. | Start.Coord | End.Coord | Strand | Scaffold.ID |
|---|---|---|---|---|
| 1 | 5509888 | 5510901 | + | 640753026 |
| 2 | 5497616 | 5498380 | − | 640753026 |
| 3 | 269 | 829 | + | 649999021 |
| 4 | 2600850 | 2601410 | − | 641522670 |
| 5 | 112316 | 112732 | + | 640753026 |
| 6 | 1298354 | 1298788 | + | 650716170 |
| 7 | 5930458 | 5930823 | − | 650716164 |
| 8 | 29403 | 29642 | − | 649999069 |
| 9 | 9109 | 10122 | − | 638362473 |
| 10 | 35241 | 35606 | − | 649999019 |
| 11 | 25607 | 28270 | + | 649999084 |
| 12 | 18240 | 19688 | − | 638362609 |
| 13 | 212874 | 213821 | − | 649998930 |
| 14 | 22370 | 34666 | + | 638362642 |
| 15 | 122819 | 123664 | + | 640753026 |
| 16 | 3864574 | 3865185 | − | 640753026 |
| 17 | 35624 | 37309 | − | 649999019 |
| 18 | 5409448 | 5411001 | − | 640753026 |
| 19 | 2597880 | 2600846 | − | 641522670 |
| 20 | 4961575 | 4962753 | − | 640753026 |
| 21 | 167495 | 167935 | + | 638362589 |
| 22 | 28 | 241 | − | 647002430 |
| 23 | 1310460 | 1310735 | − | 639279323 |
| 24 | 161059 | 161934 | + | 638362589 |
| 25 | 3500513 | 3501292 | + | 638372930 |
| 26 | 2885013 | 2885837 | − | 643348637 |
| 27 | 3505406 | 3506134 | + | 638372930 |
| 28 | 182786 | 184474 | + | 638362589 |
| 29 | 366452 | 367480 | − | 640753026 |
| 30 | 26408 | 27688 | − | 649999069 |
| 31 | 4403159 | 4405534 | − | 639279323 |
| 32 | 3528459 | 3529049 | + | 638372930 |
| 33 | 3855983 | 3856195 | − | 640753026 |
| 34 | 121405 | 121680 | − | 640753026 |
| 35 | 179158 | 179895 | + | 638362589 |
| 36 | 48194 | 50227 | + | 649999006 |
| 37 | 3507452 | 3509473 | + | 638372930 |
| 38 | 3547764 | 3548102 | + | 638372930 |
| 39 | 163874 | 164434 | + | 638362589 |
| 40 | 2856655 | 2857038 | − | 643348637 |
| 41 | 1367161 | 1367601 | − | 651053081 |
| 42 | 3506732 | 3507172 | + | 638372930 |
| 43 | 6653 | 8650 | − | 649999070 |
| 44 | 2001 | 4451 | − | 649999070 |
| 45 | 2683111 | 2683836 | − | 639279323 |
| 46 | 138340 | 138654 | − | 638362591 |
| 47 | 214185 | 216386 | + | 638362589 |
| 48 | 3640230 | 3642680 | − | 643692060 |
| 49 | 4730 | 5116 | − | 649999070 |
| 50 | 3833901 | 3834893 | − | 640753026 |

### Example 9

### Salmonella species

The procedure was carried out as in Example 1, except that the following microorganisms were used:

Bacterial Strains

The inventors selected 634 Salmonella strains, particularly from Salmonella_choleraesuis, Salmonella_dublin, Salmonella_enterica_ssp_arizonae, Salmonella_enterica_ssp_diarizoniae, Salmonella_enteritidis, Salmonella_gallinarum, Salmonella Group_A, Salmonella_Group_B, Salmonella_Group_C, Salmonella_Group_D, Salmonella_heidelberg, Salmonella_miami, Salmonella_newport, Salmonella_panama, Salmonella_parahaemolyticus_A, Salmonella_paratyphi_A, Salmonella_paratyphi_B, Salmonella_pullorum, Salmonella_senfienberg, Salmonella species, Salmonella_species_Lac_−−,_ONPG_+, Salmonella_species_Lac_+,_ONPG_+, Salmonella subgenus_I, Salmonella_subgenus_II, Salmonella_subgenus_IV, Salmonella subgroup_I_Suc+, Salmonella_tennessee, and Salmonella_typhi, from the microbiology strain collection at Siemens Healthcare Diagnostics (West Sacramento, Calif.) for susceptibility testing and whole genome sequencing.

From MetaRef, 713 centroids of Salmonella species were used as reference sequences.

The results for Salmonella species are shown in Tables 17 (corresponding to Table 1) and 18 (corresponding to Table 2).

TABLE 17a
Data for Salmonella species

| | Scaffold.External. | | |
|---|---|---|---|
| No. | Accession | Scaffold.Name | best_pv |
| 1 | NC_011083 | Salmonella enterica subsp. enterica serovar Heidelberg str. SL476: | 1.09817680047592e−59 |
| | | NC_011083 | |
| 2 | NC_011092 | Salmonella enterica subsp. enterica serovar Schwarzengrund str. | 3.48405564494782e−42 |
| | | CVM19633 plasmid pCVM19633_110: NC_011092 | |
| 3 | NZ_ADUB01000261 | Escherichia coli MS 175-1 E_coli175-1-1.0_Cont542.3: NZ_ADUB01000261 | 2.50701590062067e−24 |
| 4 | NC_009651 | Klebsiella pneumoniae subsp. pneumoniae MGH 78578 plasmid pKPN5: | 3.64875434048827e−22 |
| | | NC_009651 | |
| 5 | NC_013509 | Edwardsiella tarda EIB202 plasmid pEIB202: NC_013509 | 1.44445847616457e−21 |
| 6 | NZ_ABFH01000001 | Salmonella enterica subsp. enterica serovar Virchow str. SL491, | 3.48310596778991e−21 |
| | | unfinished sequence: NZ_ABFH01000001 | |
| 7 | NC_011743 | Escherichia fergusonii ATCC 35469 plasmid pEFER: NC_011743 | 3.48310596778991e−21 |
| 8 | NZ_ADWV01000045 | Escherichia coli MS 107-1 E_coliMS107-1-1.0.1_Cont44.1: | 1.74745925214862e−20 |
| | | NZ_ADWV01000045 | |
| 9 | NC_013509 | Edwardsiella tarda EIB202 plasmid pEIB202: NC_013509 | 3.3130773442785e−20 |
| 10 | NZ_AAJX01000089 | Escherichia coli B171, unfinished sequence: NZ_AAJX01000089 | 5.485221922018e−20 |
| 11 | NZ_AFBO01000625 | Klebsiella sp. MS 92-3 K_spMS92-3-1.0_Cont1175.2: NZ_AFBO01000625 | 7.41649956052229e−17 |
| 12 | NZ_ADTM01000311 | Escherichia coli MS 182-1 E_coli182-1-1.0_Cont460.3: NZ_ADTM01000311 | 8.87457605302667e−17 |
| 13 | CP001383 | Shigella flexneri 2002017: CP001383 | 5.28841759700461e−14 |
| 14 | NC_007384 | Shigella sonnei Ss046: NC_007384 | 5.8995130852235e−14 |
| 15 | NZ_ADUD01000537 | Escherichia coli MS 196-1 E_coli196-1-1.0_Cont1482.1: | 1.65276555063276e−13 |
| | | NZ_ADUD01000537 | |
| 16 | NZ_ABFH01000001 | Salmonella enterica subsp. enterica serovar Virchow str. SL491, | 1.65276555063276e−13 |
| | | unfinished sequence: NZ_ABFH01000001 | |
| 17 | NC_009650 | Klebsiella pneumoniae subsp. pneumoniae MGH 78578 plasmid pKPN4: | 3.61473686772869e−13 |
| | | NC_009650 | |
| 18 | NZ_ABFH01000001 | Salmonella enterica subsp. enterica serovar Virchow str. SL491, | 5.18796174101329e−13 |
| | | unfinished sequence: NZ_ABFH01000001 | |
| 19 | NZ_ADWV01000017 | Escherichia coli MS 107-1 E_coliMS107-1-1.0.1_Cont16.1: | 3.37689513091676e−12 |
| | | NZ_ADWV01000017 | |
| 20 | NZ_ADWT01000152 | Escherichia coli MS 124-1 E_coliMS124-1-1.0.1_Cont151.1: | 5.32683257781776e−12 |
| | | NZ_ADWT01000152 | |
| 21 | CP001383 | Shigella flexneri 2002017: CP001383 | 9.59412420701724e−12 |
| 22 | NC_011081 | Salmonella enterica subsp. enterica serovar Heidelberg str. SL476 | 1.80214883490127e−11 |
| | | plasmid pSL476_91: NC_011081 | |
| 23 | AP011957 | Salmonella enterica subsp. enterica serovar Typhimurium str. T000240 | 2.02729069966733e−11 |
| | | DNA: AP011957 | |
| 24 | NC_009650 | Klebsiella pneumoniae subsp. pneumoniae MGH 78578 plasmid pKPN4: | 2.02729069966733e−11 |
| | | NC_009650 | |
| 25 | NC_011092 | Salmonella enterica subsp. enterica serovar Schwarzengrund str. | 3.62500325966221e−11 |
| | | CVM19633 plasmid pCVM19633_110: NC_011092 | |
| 26 | NZ_ABKX01000001 | Escherichia albertii TW07627, unfinished sequence: NZ_ABKX01000001 | 5.50212585421897e−11 |
| 27 | CP002490 | Salmonella enterica subsp. enterica serovar Typhimurium str. 4/74 | 1.68214594408478e−10 |
| | | plasmid TY474p3: CP002490 | |
| 28 | NC_012124 | Salmonella enterica subsp. enterica serovar Paratyphi C strain | 8.57828076933948e−10 |
| | | RKS4594 plasmid pSPCV: NC_012124 | |
| 29 | NC_007385 | Shigella sonnei Ss046 plasmid pSS_046: NC_007385 | 1.06330274297554e−09 |
| 30 | NC_004851 | Shigella flexneri 2a str. 301 plasmid pCP301: NC_004851 | 2.2269317280314e−09 |
| 31 | NC_003277 | Salmonella typhimurium LT2 plasmid pSLT: NC_003277 | 2.2269317280314e−09 |
| 32 | CP001362 | Salmonella enterica subsp. enterica serovar Typhimurium str. 14028S | 1.72272938882188e−08 |
| | | plasmid: CP001362 | |
| 33 | NZ_ABKX01000001 | Escherichia albertii TW07627, unfinished sequence: NZ_ABKX01000001 | 1.8184047282463e−08 |
| 34 | NC_012124 | Salmonella enterica subsp. enterica serovar Paratyphi C strain | 3.5232749457209e−08 |
| | | RKS4594 plasmid pSPCV: NC_012124 | |
| 35 | NC_012125 | Salmonella enterica subsp. enterica serovar Paratyphi C strain | 4.21301623299358e−08 |
| | | RKS4594: NC_012125 | |
| 36 | CP002487 | Salmonella enterica subsp. enterica serovar Typhimurium str. 4/74: | 5.49621728459414e−08 |
| | | CP002487 | |
| 37 | NZ_ABEL01000010 | Salmonella enterica subsp. enterica serovar Heidelberg str. SL486, | 9.94168564125988e−08 |
| | | unfinished sequence: NZ_ABEL01000010 | |
| 38 | NC_012124 | Salmonella enterica subsp. enterica serovar Paratyphi C strain | 1.81471391611537e−07 |
| | | RKS4594 plasmid pSPCV: NC_012124 | |
| 39 | NC_012125 | Salmonella enterica subsp. enterica serovar Paratyphi C strain | 1.9714526298817e−07 |
| | | RKS4594: NC_012125 | |
| 40 | NC_003197 | Salmonella typhimurium LT2: NC_003197 | 2.16264005553646e−07 |
| 41 | CP002488 | Salmonella enterica subsp. enterica serovar Typhimurium str. 4/74 | 2.16264005553646e−07 |
| | | plasmid TY474p1: CP002488 | |
| 42 | NZ_ABAM01000046 | Salmonella enterica subsp. enterica serovar Saintpaul str. SARA23, | 2.29779633234294e−07 |
| | | unfinished sequence: NZ_ABAM01000046 | |
| 43 | NC_010102 | Salmonella enterica subsp. enterica serovar Paratyphi B str. SPB7: | 3.05541326329248e−07 |
| | | NC_010102 | |
| 44 | AP011957 | Salmonella enterica subsp. enterica serovar Typhimurium str. T000240 | 3.48861961356893e−07 |
| | | DNA: AP011957 | |
| 45 | NC_010067 | Salmonella enterica subsp. arizonae serovar 62:z4,z23:--: NC_010067 | 4.35325104483177e−07 |
| 46 | NC_010067 | Salmonella enterica subsp. arizonae serovar 62:z4,z23:--: NC_010067 | 4.35325104483177e−07 |
| 47 | NC_015761 | Salmonella bongori NCTC 12419: NC_015761 | 4.35325104483177e−07 |
| 48 | NZ_ABEL01000010 | Salmonella enterica subsp. enterica serovar Heidelberg str. SL486, | 7.61833017744037e−07 |
| | | unfinished sequence: NZ_ABEL01000010 | |
| 49 | NC_012125 | Salmonella enterica subsp. enterica serovar Paratyphi C strain | 8.71713912669417e−07 |
| | | RKS4594: NC_012125 | |
| 50 | NZ_ABEJ01000032 | Salmonella enterica subsp. enterica serovar Schwarzengrund str. | 8.71713912669417e−07 |
| | | SL480, unfinished sequence: NZ_ABEJ01000032 | |

TABLE 17b
Data for Salmonella species (continued)

| No. | sign_phenos | sign_phenos_class | best_pheno | best_pheno_class |
|---|---|---|---|---|
| 1 | AM; A/S | lactam | AM | lactam |
| 2 | AM; A/S | lactam | AM | lactam |
| 3 | AM; A/S | lactam | AM | lactam |
| 4 | AM; A/S | lactam | A/S | lactam |
| 5 | AM; A/S | lactam | AM | lactam |
| 6 | AM; A/S | lactam | A/S | lactam |
| 7 | AM; A/S | lactam | AM | lactam |
| 8 | AM; A/S | lactam | A/S | lactam |
| 9 | AM; A/S | lactam | AM | lactam |
| 10 | AM; A/S | lactam | A/S | lactam |
| 11 | AM; A/S | lactam | A/S | lactam |
| 12 | AM; A/S | lactam | A/S | lactam |
| 13 | AM; A/S | lactam | AM | lactam |
| 14 | AM; A/S | lactam | A/S | lactam |
| 15 | AM; A/S | lactam | A/S | lactam |
| 16 | AM; A/S | lactam | A/S | lactam |
| 17 | AM; A/S | lactam | A/S | lactam |
| 18 | AM; A/S | lactam | A/S | lactam |
| 19 | AM; A/S | lactam | A/S | lactam |
| 20 | AM; A/S | lactam | AM | lactam |
| 21 | AM; A/S | lactam | AM | lactam |
| 22 | AM; A/S | lactam | AM | lactam |
| 23 | AM; A/S | lactam | A/S | lactam |
| 24 | AM; A/S | lactam | A/S | lactam |
| 25 | AM; A/S | lactam | A/S | lactam |
| 26 | AM; A/S | lactam | AM | lactam |
| 27 | AM; A/S | lactam | AM | lactam |
| 28 | AM; A/S | lactam | AM | lactam |
| 29 | AM; A/S | lactam | AM | lactam |
| 30 | AM; A/S | lactam | AM | lactam |
| 31 | AM; A/S | lactam | AM | lactam |
| 32 | AM; A/S | lactam | AM | lactam |
| 33 | AM; A/S | lactam | AM | lactam |
| 34 | AM; A/S | lactam | AM | lactam |
| 35 | AM; A/S | lactam | AM | lactam |
| 36 | AM; A/S | lactam | AM | lactam |
| 37 | AM; A/S | lactam | A/S | lactam |
| 38 | AM; A/S | lactam | AM | lactam |
| 39 | AM; A/S | lactam | AM | lactam |
| 40 | AM; A/S | lactam | AM | lactam |
| 41 | AM; A/S | lactam | AM | lactam |
| 42 | AM; A/S | lactam | A/S | lactam |
| 43 | AM; A/S | lactam | AM | lactam |
| 44 | AM; A/S | lactam | A/S | lactam |
| 45 | AM; A/S | lactam | A/S | lactam |
| 46 | AM; A/S | lactam | A/S | lactam |
| 47 | AM; A/S | lactam | A/S | lactam |
| 48 | AM; A/S | lactam | A/S | lactam |
| 49 | AM; A/S | lactam | A/S | lactam |
| 50 | AM; A/S | lactam | A/S | lactam |

TABLE 17c
Data for Salmonella species (continued)

| No. | num_lactam | AM_pv_adj | A/S_pv_adj |
|---|---|---|---|
| 1 | 2 | 1.10E−59 | 1.70E−55 |
| 2 | 2 | 3.48E−42 | 1.81E−40 |
| 3 | 2 | 2.51E−24 | 2.30E−22 |
| 4 | 2 | 1.08E−21 | 3.65E−22 |
| 5 | 2 | 1.44E−21 | 6.88E−20 |
| 6 | 2 | 1.05E−20 | 3.48E−21 |
| 7 | 2 | 3.48E−21 | 1.05E−20 |
| 8 | 2 | 5.22E−20 | 1.75E−20 |
| 9 | 2 | 3.31E−20 | 1.15E−19 |
| 10 | 2 | 1.65E−19 | 5.49E−20 |
| 11 | 2 | 1.74E−16 | 7.42E−17 |
| 12 | 2 | 2.98E−16 | 8.87E−17 |
| 13 | 2 | 5.29E−14 | 1.45E−13 |
| 14 | 2 | 1.45E−13 | 5.90E−14 |
| 15 | 2 | 3.62E−13 | 1.65E−13 |
| 16 | 2 | 3.62E−13 | 1.65E−13 |
| 17 | 2 | 6.38E−13 | 3.61E−13 |
| 18 | 2 | 1.10E−12 | 5.19E−13 |
| 19 | 2 | 6.91E−12 | 3.38E−12 |
| 20 | 2 | 5.33E−12 | 3.21E−11 |
| 21 | 2 | 9.59E−12 | 8.71E−10 |
| 22 | 2 | 1.80E−11 | 6.37E−10 |
| 23 | 2 | 3.64E−11 | 2.03E−11 |
| 24 | 2 | 3.64E−11 | 2.03E−11 |
| 25 | 2 | 6.67E−11 | 3.63E−11 |
| 26 | 2 | 5.50E−11 | 4.78E−09 |
| 27 | 2 | 1.68E−10 | 1.06E−09 |
| 28 | 2 | 8.58E−10 | 2.16E−08 |
| 29 | 2 | 1.06E−09 | 2.10E−08 |
| 30 | 2 | 2.23E−09 | 3.77E−08 |
| 31 | 2 | 2.23E−09 | 3.77E−08 |
| 32 | 2 | 1.72E−08 | 2.02E−07 |
| 33 | 2 | 1.82E−08 | 1.76E−06 |
| 34 | 2 | 3.52E−08 | 8.59E−08 |
| 35 | 2 | 4.21E−08 | 6.33E−08 |
| 36 | 2 | 5.50E−08 | 7.64E−08 |
| 37 | 2 | 1.40E−07 | 9.94E−08 |
| 38 | 2 | 1.81E−07 | 3.88E−07 |
| 39 | 2 | 1.97E−07 | 2.71E−07 |
| 40 | 2 | 2.16E−07 | 2.91E−07 |
| 41 | 2 | 2.16E−07 | 7.75E−07 |
| 42 | 2 | 7.51E−07 | 2.30E−07 |
| 43 | 2 | 3.06E−07 | 8.87E−07 |
| 44 | 2 | 2.22E−06 | 3.49E−07 |
| 45 | 2 | 1.28E−06 | 4.35E−07 |
| 46 | 2 | 1.28E−06 | 4.35E−07 |
| 47 | 2 | 1.25E−06 | 4.35E−07 |
| 48 | 2 | 2.83E−06 | 7.62E−07 |
| 49 | 2 | 1.23E−06 | 8.72E−07 |
| 50 | 2 | 4.30E−06 | 8.72E−07 |

TABLE 17d
Data for Salmonella species (continued)

| No. | Locus.Tag | Gene.Symbol | GenBank.Accession | Chromosome |
|---|---|---|---|---|
| 1 | SeHA_C1580 | blaT | YP_002045448 | |
| 2 | SeSA_B0079 | | YP_002112958 | plasmid pCVM19633_110 |
| 3 | HMPREF9547_03104 | | ZP_07169558 | |
| 4 | KPN_pKPN5p08201 | | YP_001338811 | plasmid pKPN5 |
| 5 | ETAE_p041 | folP | YP_003297635 | plasmid pEIB202 |
| 6 | Salmoentericaenterica_010100000245 | | ZP_02702077 | |
| 7 | pEFER_0049 | | YP_002394596 | plasmid pEFER |
| 8 | HMPREF9345_05066 | | ZP_07100147 | |
| 9 | ETAE_p040 | | YP_003297634 | plasmid pEIB202 |
| 10 | EcolB_01004576 | | ZP_00708527 | |
| 11 | HMPREF9538_04737 | | ZP_08307036 | |
| 12 | HMPREF9548_03784 | | ZP_07141588 | |
| 13 | SFxv_1142 | | ADA73400 | |
| 14 | SSON_3896 | aadA | YP_312670 | |
| 15 | HMPREF9551_04611 | | ZP_07191964 | |
| 16 | Salmoentericaenterica_010100000130 | | ZP_02702054 | |
| 17 | KPN_pKPN4p07064 | | YP_001338673 | plasmid pKPN4 |
| 18 | Salmoentericaenterica_010100000105 | | ZP_02702049 | |
| 19 | HMPREF9345_03525 | | ZP_07098656 | |
| 20 | HMPREF9347_06051 | | ZP_07213485 | |
| 21 | SFxv_1141 | | ADA73399 | |
| 22 | SeHA_A0031 | | YP_002043869 | plasmid pSL476_91 |
| 23 | STMDT12_C39390 | | BAJ38882 | |
| 24 | KPN_pKPN4p07068 | merC | YP_001338677 | plasmid pKPN4 |
| 25 | SeSA_B0082 | | YP_002112961 | plasmid pCVM19633_110 |
| 26 | ESCAB7627_0644 | | ZP_02900823 | |
| 27 | STM474_p303 | repA | ADX20580 | plasmid TY474p3 |
| 28 | SPC_p013 | rsdB | YP_002635587 | plasmid pSPCV |
| 29 | SSO_P190 | traD | YP_313445 | plasmid pSS_046 |
| 30 | CP0245 | mvpA | NP_858378 | plasmid pCP301 |
| 31 | PSLT105 | trbH | NP_490589 | plasmid pSLT |
| 32 | STM14_5622 | traD | ACY86531 | |
| 33 | ESCAB7627_0650 | | ZP_02900555 | |
| 34 | SPC_p042 | traX | YP_002635616 | plasmid pSPCV |
| 35 | SPC_2707 | | YP_002638248 | |
| 36 | STM474_1038 | | ADX16744 | |
| 37 | Senterienterica_010100019467 | | ZP_02668762 | |
| 38 | SPC_p041 | finO | YP_002635615 | plasmid pSPCV |
| 39 | SPC_2708 | | YP_002638249 | |
| 40 | STM1046 | | NP_460021 | |
| 41 | STM474_p1093 | ccdB | ADX20463 | plasmid TY474p1 |
| 42 | Sentent_010100022581 | | ZP_02341659 | |
| 43 | SPAB_05276 | | YP_001591385 | |
| 44 | STMDT12_C10780 | | BAJ36021 | |
| 45 | SARI_01515 | | YP_001570553 | |
| 46 | SARI_01514 | | YP_001570552 | |
| 47 | SBG_1292 | malR | YP_004730162 | |
| 48 | Senterienterica_010100019192 | | ZP_02668707 | |
| 49 | SPC_0340 | | YP_002635966 | |
| 50 | Senteenterica_010100025269 | | ZP_02664569 | |

TABLE 17e
Data for Salmonella species (continued)

| No. | Start.Coord | End.Coord | Strand | Scaffold.ID |
|---|---|---|---|---|
| 1 | 1525960 | 1526820 | + | 642555218 |
| 2 | 68933 | 70240 | + | 642555219 |
| 3 | 3248 | 3841 | + | 648291919 |
| 4 | 34908 | 35255 | − | 640753021 |
| 5 | 32101 | 32916 | − | 646311959 |
| 6 | 36757 | 37062 | + | 641778223 |
| 7 | 48658 | 49503 | + | 643692014 |
| 8 | 4715 | 5554 | − | 648294624 |
| 9 | 31237 | 32040 | − | 646311959 |
| 10 | 3555 | 4568 | + | 638359041 |
| 11 | 1555 | 2055 | − | 651333542 |
| 12 | 8819 | 9535 | + | 648288593 |
| 13 | 1108351 | 1108854 | − | 646862363 |
| 14 | 4110458 | 4111246 | − | 640427102 |
| 15 | 97 | 531 | − | 648293053 |
| 16 | 19257 | 19691 | + | 641778223 |
| 17 | 23310 | 24614 | − | 640753020 |
| 18 | 16271 | 16636 | − | 641778223 |
| 19 | 1071 | 1307 | − | 648294596 |
| 20 | 812 | 1663 | − | 648294425 |
| 21 | 1107342 | 1107968 | + | 646862363 |
| 22 | 16416 | 17195 | + | 642555216 |
| 23 | 4088051 | 4088326 | − | 651053007 |
| 24 | 25970 | 27664 | − | 640753020 |
| 25 | 71061 | 71411 | − | 642555219 |
| 26 | 233492 | 234697 | − | 641784575 |
| 27 | 1988 | 2827 | − | 650378006 |
| 28 | 10060 | 10842 | − | 643692029 |
| 29 | 166425 | 168626 | + | 640427103 |
| 30 | 198117 | 198515 | − | 637000224 |
| 31 | 86566 | 87285 | + | 637000134 |
| 32 | 84297 | 86459 | + | 646862361 |
| 33 | 238788 | 239996 | − | 641784575 |
| 34 | 28413 | 29153 | − | 643692029 |
| 35 | 2745099 | 2745596 | − | 643692030 |
| 36 | 1088568 | 1089272 | + | 650378003 |
| 37 | 62341 | 62874 | − | 641777794 |
| 38 | 27795 | 28358 | − | 643692029 |
| 39 | 2745695 | 2746027 | − | 643692030 |
| 40 | 1131381 | 1132118 | + | 637000122 |
| 41 | 73221 | 73526 | − | 650378004 |
| 42 | 23575 | 23910 | − | 641746440 |
| 43 | 4445787 | 4448009 | + | 641228523 |
| 44 | 1141208 | 1141399 | − | 651053007 |
| 45 | 1476854 | 1478026 | + | 641228515 |
| 46 | 1475252 | 1476844 | + | 641228515 |
| 47 | 1414087 | 1415115 | + | 650716177 |
| 48 | 26832 | 28046 | + | 641777794 |
| 49 | 367421 | 368839 | + | 643692030 |
| 50 | 35858 | 36535 | − | 641777693 |

TABLE 18a
Data for Salmonella species

| | Scaffold.External. | | |
|---|---|---|---|
| No. | Accession | Scaffold.Name | best_pv |
| 1 | NC_011083 | Salmonella enterica subsp. enterica serovar Heidelberg str. SL476: | 1.09817680047592e−59 |
| | | NC_011083 | |
| 2 | NC_011092 | Salmonella enterica subsp. enterica serovar Schwarzengrund str. | 3.48405564494782e−42 |
| | | CVM19633 plasmid pCVM19633_110: NC_011092 | |
| 3 | NZ_ADUB01000261 | Escherichia coli MS 175-1 E_coli175-1-1.0_Cont542.3: NZ_ADUB01000261 | 2.50701590062067e−24 |
| 4 | NC_009651 | Klebsiella pneumoniae subsp. pneumoniae MGH 78578 plasmid pKPN5: | 3.64875434048827e−22 |
| | | NC_009651 | |
| 5 | NC_013509 | Edwardsiella tarda EIB202 plasmid pEIB202: NC_013509 | 1.44445847616457e−21 |
| 6 | NZ_ABFH01000001 | Salmonella enterica subsp. enterica serovar Virchow str. SL491, | 3.48310596778991e−21 |
| | | unfinished sequence: NZ_ABFH01000001 | |
| 7 | NC_011743 | Escherichia fergusonii ATCC 35469 plasmid pEFER: NC_011743 | 3.48310596778991e−21 |
| 8 | NZ_ADWV01000045 | Escherichia coli MS 107-1 E_coliMS107-1-1.0.1_Cont44.1: | 1.74745925214862e−20 |
| | | NZ_ADWV01000045 | |
| 9 | NC_013509 | Edwardsiella tarda EIB202 plasmid pEIB202: NC_013509 | 3.3130773442785e−20 |
| 10 | NZ_AAJX01000089 | Escherichia coli B171, unfinished sequence: NZ_AAJX01000089 | 5.485221922018e−20 |
| 11 | NZ_AFBO01000625 | Klebsiella sp. MS 92-3 K_spMS92-3-1.0_Cont1175.2: NZ_AFBO01000625 | 7.41649956052229e−17 |
| 12 | NZ_ADTM01000311 | Escherichia coli MS 182-1 E_coli182-1-1.0_Cont460.3: NZ_ADTM01000311 | 8.87457605302667e−17 |
| 13 | CP001383 | Shigella flexneri 2002017: CP001383 | 5.28841759700461e−14 |
| 14 | NC_007384 | Shigella sonnei Ss046: NC_007384 | 5.8995130852235e−14 |
| 15 | NZ_ADUD01000537 | Escherichia coli MS 196-1 E_coli196-1-1.0_Cont1482.1: NZ_ADUD01000537 | 1.65276555063276e−13 |
| 16 | NZ_ABFH01000001 | Salmonella enterica subsp. enterica serovar Virchow str. SL491, | 1.65276555063276e−13 |
| | | unfinished sequence: NZ_ABFH01000001 | |
| 17 | NC_009650 | Klebsiella pneumoniae subsp. pneumoniae MGH 78578 plasmid pKPN4: | 3.61473686772869e−13 |
| | | NC_009650 | |
| 18 | NZ_ABFH01000001 | Salmonella enterica subsp. enterica serovar Virchow str. SL491, | 5.18796174101329e−13 |
| | | unfinished sequence: NZ_ABFH01000001 | |
| 19 | NZ_ADWV01000017 | Escherichia coli MS 107-1 E_coliMS107-1-1.0.1_Cont16.1: | 3.37689513091676e−12 |
| | | NZ_ADWV01000017 | |
| 20 | NZ_ADWT01000152 | Escherichia coli MS 124-1 E_coliMS124-1-1.0.1_Cont151.1: | 5.32683257781776e−12 |
| | | NZ_ADWT01000152 | |
| 21 | CP001383 | Shigella flexneri 2002017: CP001383 | 9.59412420701724e−12 |
| 22 | NC_011081 | Salmonella enterica subsp. enterica serovar Heidelberg str. SL476 | 1.80214883490127e−11 |
| | | plasmid pSL476_91: NC_011081 | |
| 23 | AP011957 | Salmonella enterica subsp. enterica serovar Typhimurium str. T000240 | 2.02729069966733e−11 |
| | | DNA: AP011957 | |
| 24 | NC_009650 | Klebsiella pneumoniae subsp. pneumoniae MGH 78578 plasmid pKPN4: | 2.02729069966733e−11 |
| | | NC_009650 | |
| 25 | NC_011092 | Salmonella enterica subsp. enterica serovar Schwarzengrund str. | 3.62500325966221e−11 |
| | | CVM19633 plasmid pCVM19633_110: NC_011092 | |
| 26 | NZ_ABKX01000001 | Escherichia albertii TW07627, unfinished sequence: NZ_ABKX01000001 | 5.50212585421897e−11 |
| 27 | CP002490 | Salmonella enterica subsp. enterica serovar Typhimurium str. 4/74 | 1.68214594408478e−10 |
| | | plasmid TY474p3: CP002490 | |
| 28 | NC_012124 | Salmonella enterica subsp. enterica serovar Paratyphi C strain RKS4594 | 8.57828076933948e−10 |
| | | plasmid pSPCV: NC_012124 | |
| 29 | NC_007385 | Shigella sonnei Ss046 plasmid pSS_046: NC_007385 | 1.06330274297554e−09 |
| 30 | NC_004851 | Shigella flexneri 2a str. 301 plasmid pCP301: NC_004851 | 2.2269317280314e−09 |
| 31 | NC_003277 | Salmonella typhimurium LT2 plasmid pSLT: NC_003277 | 2.2269317280314e−09 |
| 32 | CP001362 | Salmonella enterica subsp. enterica serovar Typhimurium str. 14028S | 1.72272938882188e−08 |
| | | plasmid: CP001362 | |
| 33 | NZ_ABKX01000001 | Escherichia albertii TW07627, unfinished sequence: NZ_ABKX01000001 | 1.8184047282463e−08 |
| 34 | NC_012124 | Salmonella enterica subsp. enterica serovar Paratyphi C strain RKS4594 | 3.5232749457209e−08 |
| | | plasmid pSPCV: NC_012124 | |
| 35 | NC_012125 | Salmonella enterica subsp. enterica serovar Paratyphi C strain RKS4594: | 4.21301623299358e−08 |
| | | NC_012125 | |
| 36 | CP002487 | Salmonella enterica subsp. enterica serovar Typhimurium str. 4/74: | 5.49621728459414e−08 |
| | | CP002487 | |
| 37 | NZ_ABEL01000010 | Salmonella enterica subsp. enterica serovar Heidelberg str. SL486, | 9.94168564125988e−08 |
| | | unfinished sequence: NZ_ABEL01000010 | |
| 38 | NC_012124 | Salmonella enterica subsp. enterica serovar Paratyphi C strain RKS4594 | 1.81471391611537e−07 |
| | | plasmid pSPCV: NC_012124 | |
| 39 | NC_012125 | Salmonella enterica subsp. enterica serovar Paratyphi C strain RKS4594: | 1.9714526298817e−07 |
| | | NC_012125 | |
| 40 | NC_003197 | Salmonella typhimurium LT2: NC_003197 | 2.16264005553646e−07 |
| 41 | CP002488 | Salmonella enterica subsp. enterica serovar Typhimurium str. 4/74 | 2.16264005553646e−07 |
| | | plasmid TY474p1: CP002488 | |
| 42 | NZ_ABAM01000046 | Salmonella enterica subsp. enterica serovar Saintpaul str. SARA23, | 2.29779633234294e−07 |
| | | unfinished sequence: NZ_ABAM01000046 | |
| 43 | NC_010102 | Salmonella enterica subsp. enterica serovar Paratyphi B str. SPB7: | 3.05541326329248e−07 |
| | | NC_010102 | |
| 44 | AP011957 | Salmonella enterica subsp. enterica serovar Typhimurium str. T000240 | 3.48861961356893e−07 |
| | | DNA: AP011957 | |
| 45 | NC_010067 | Salmonella enterica subsp. arizonae serovar 62:z4,z23:--: NC_010067 | 4.35325104483177e−07 |
| 46 | NC_010067 | Salmonella enterica subsp. arizonae serovar 62:z4,z23:--: NC_010067 | 4.35325104483177e−07 |
| 47 | NC_015761 | Salmonella bongori NCTC 12419: NC_015761 | 4.35325104483177e−07 |
| 48 | NZ_ABEL01000010 | Salmonella enterica subsp. enterica serovar Heidelberg str. SL486, | 7.61833017744037e−07 |
| | | unfinished sequence: NZ_ABEL01000010 | |
| 49 | NC_012125 | Salmonella enterica subsp. enterica serovar Paratyphi C strain RKS4594: | 8.71713912669417e−07 |
| | | NC_012125 | |
| 50 | NZ_ABEJ01000032 | Salmonella enterica subsp. enterica serovar Schwarzengrund str. SL480, | 8.71713912669417e−07 |
| | | unfinished sequence: NZ_ABEJ01000032 | |

TABLE 18b
Data for Salmonella species (continued)

| No. | sign_phenos | sign_phenos_class | best_pheno | best_pheno_class |
|---|---|---|---|---|
| 1 | AM; A/S | lactam | AM | lactam |
| 2 | AM; A/S | lactam | AM | lactam |
| 3 | AM; A/S | lactam | AM | lactam |
| 4 | AM; A/S | lactam | A/S | lactam |
| 5 | AM; A/S | lactam | AM | lactam |
| 6 | AM; A/S | lactam | A/S | lactam |
| 7 | AM; A/S | lactam | AM | lactam |
| 8 | AM; A/S | lactam | A/S | lactam |
| 9 | AM; A/S | lactam | AM | lactam |
| 10 | AM; A/S | lactam | A/S | lactam |
| 11 | AM; A/S | lactam | A/S | lactam |
| 12 | AM; A/S | lactam | A/S | lactam |
| 13 | AM; A/S | lactam | AM | lactam |
| 14 | AM; A/S | lactam | A/S | lactam |
| 15 | AM; A/S | lactam | A/S | lactam |
| 16 | AM; A/S | lactam | A/S | lactam |
| 17 | AM; A/S | lactam | A/S | lactam |
| 18 | AM; A/S | lactam | A/S | lactam |
| 19 | AM; A/S | lactam | A/S | lactam |
| 20 | AM; A/S | lactam | AM | lactam |
| 21 | AM; A/S | lactam | AM | lactam |
| 22 | AM; A/S | lactam | AM | lactam |
| 23 | AM; A/S | lactam | A/S | lactam |
| 24 | AM; A/S | lactam | A/S | lactam |
| 25 | AM; A/S | lactam | A/S | lactam |
| 26 | AM; A/S | lactam | AM | lactam |
| 27 | AM; A/S | lactam | AM | lactam |
| 28 | AM; A/S | lactam | AM | lactam |
| 29 | AM; A/S | lactam | AM | lactam |
| 30 | AM; A/S | lactam | AM | lactam |
| 31 | AM; A/S | lactam | AM | lactam |
| 32 | AM; A/S | lactam | AM | lactam |
| 33 | AM; A/S | lactam | AM | lactam |
| 34 | AM; A/S | lactam | AM | lactam |
| 35 | AM; A/S | lactam | AM | lactam |
| 36 | AM; A/S | lactam | AM | lactam |
| 37 | AM; A/S | lactam | A/S | lactam |
| 38 | AM; A/S | lactam | AM | lactam |
| 39 | AM; A/S | lactam | AM | lactam |
| 40 | AM; A/S | lactam | AM | lactam |
| 41 | AM; A/S | lactam | AM | lactam |
| 42 | AM; A/S | lactam | A/S | lactam |
| 43 | AM; A/S | lactam | AM | lactam |
| 44 | AM; A/S | lactam | A/S | lactam |
| 45 | AM; A/S | lactam | A/S | lactam |
| 46 | AM; A/S | lactam | A/S | lactam |
| 47 | AM; A/S | lactam | A/S | lactam |
| 48 | AM; A/S | lactam | A/S | lactam |
| 49 | AM; A/S | lactam | A/S | lactam |
| 50 | AM; A/S | lactam | A/S | lactam |

TABLE 18c
Data for Salmonella species (continued)

| No. | num_lactam | AM_pv_adj | A/S_pv_adj |
|---|---|---|---|
| 1 | 2 | 1.10E−59 | 1.70E−55 |
| 2 | 2 | 3.48E−42 | 1.81E−40 |
| 3 | 2 | 2.51E−24 | 2.30E−22 |
| 4 | 2 | 1.08E−21 | 3.65E−22 |
| 5 | 2 | 1.44E−21 | 6.88E−20 |
| 6 | 2 | 1.05E−20 | 3.48E−21 |
| 7 | 2 | 3.48E−21 | 1.05E−20 |
| 8 | 2 | 5.22E−20 | 1.75E−20 |
| 9 | 2 | 3.31E−20 | 1.15E−19 |
| 10 | 2 | 1.65E−19 | 5.49E−20 |
| 11 | 2 | 1.74E−16 | 7.42E−17 |
| 12 | 2 | 2.98E−16 | 8.87E−17 |
| 13 | 2 | 5.29E−14 | 1.45E−13 |
| 14 | 2 | 1.45E−13 | 5.90E−14 |
| 15 | 2 | 3.62E−13 | 1.65E−13 |
| 16 | 2 | 3.62E−13 | 1.65E−13 |
| 17 | 2 | 6.38E−13 | 3.61E−13 |
| 18 | 2 | 1.10E−12 | 5.19E−13 |
| 19 | 2 | 6.91E−12 | 3.38E−12 |
| 20 | 2 | 5.33E−12 | 3.21E−11 |
| 21 | 2 | 9.59E−12 | 8.71E−10 |
| 22 | 2 | 1.80E−11 | 6.37E−10 |
| 23 | 2 | 3.64E−11 | 2.03E−11 |
| 24 | 2 | 3.64E−11 | 2.03E−11 |
| 25 | 2 | 6.67E−11 | 3.63E−11 |
| 26 | 2 | 5.50E−11 | 4.78E−09 |
| 27 | 2 | 1.68E−10 | 1.06E−09 |
| 28 | 2 | 8.58E−10 | 2.16E−08 |
| 29 | 2 | 1.06E−09 | 2.10E−08 |
| 30 | 2 | 2.23E−09 | 3.77E−08 |
| 31 | 2 | 2.23E−09 | 3.77E−08 |
| 32 | 2 | 1.72E−08 | 2.02E−07 |
| 33 | 2 | 1.82E−08 | 1.76E−06 |
| 34 | 2 | 3.52E−08 | 8.59E−08 |
| 35 | 2 | 4.21E−08 | 6.33E−08 |
| 36 | 2 | 5.50E−08 | 7.64E−08 |
| 37 | 2 | 1.40E−07 | 9.94E−08 |
| 38 | 2 | 1.81E−07 | 3.88E−07 |
| 39 | 2 | 1.97E−07 | 2.71E−07 |
| 40 | 2 | 2.16E−07 | 2.91E−07 |
| 41 | 2 | 2.16E−07 | 7.75E−07 |
| 42 | 2 | 7.51E−07 | 2.30E−07 |
| 43 | 2 | 3.06E−07 | 8.87E−07 |
| 44 | 2 | 2.22E−06 | 3.49E−07 |
| 45 | 2 | 1.28E−06 | 4.35E−07 |
| 46 | 2 | 1.28E−06 | 4.35E−07 |
| 47 | 2 | 1.25E−06 | 4.35E−07 |
| 48 | 2 | 2.83E−06 | 7.62E−07 |
| 49 | 2 | 1.23E−06 | 8.72E−07 |
| 50 | 2 | 4.30E−06 | 8.72E−07 |

TABLE 18d
Data for Salmonella species (continued)

| No. | Locus.Tag | Gene.Symbol | GenBank.Accession | Chromosome |
|---|---|---|---|---|
| 1 | SeHA_C1580 | blaT | YP_002045448 | |
| 2 | SeSA_B0079 | | YP_002112958 | plasmid pCVM19633_110 |
| 3 | HMPREF9547_03104 | | ZP_07169558 | |
| 4 | KPN_pKPN5p08201 | | YP_001338811 | plasmid pKPN5 |
| 5 | ETAE_p041 | folP | YP_003297635 | plasmid pEIB202 |
| 6 | Salmoentericaenterica_010100000245 | | ZP_02702077 | |
| 7 | pEFER_0049 | | YP_002394596 | plasmid pEFER |
| 8 | HMPREF9345_05066 | | ZP_07100147 | |
| 9 | ETAE_p040 | | YP_003297634 | plasmid pEIB202 |
| 10 | EcolB_01004576 | | ZP_00708527 | |
| 11 | HMPREF9538_04737 | | ZP_08307036 | |
| 12 | HMPREF9548_03784 | | ZP_07141588 | |
| 13 | SFxv_1142 | | ADA73400 | |
| 14 | SSON_3896 | aadA | YP_312670 | |
| 15 | HMPREF9551_04611 | | ZP_07191964 | |
| 16 | Salmoentericaenterica_010100000130 | | ZP_02702054 | |
| 17 | KPN_pKPN4p07064 | | YP_001338673 | plasmid pKPN4 |
| 18 | Salmoentericaenterica_010100000105 | | ZP_02702049 | |
| 19 | HMPREF9345_03525 | | ZP_07098656 | |
| 20 | HMPREF9347_06051 | | ZP_07213485 | |
| 21 | SFxv_1141 | | ADA73399 | |
| 22 | SeHA_A0031 | | YP_002043869 | plasmid pSL476_91 |
| 23 | STMDT12_C39390 | | BAJ38882 | |
| 24 | KPN_pKPN4p07068 | merC | YP_001338677 | plasmid pKPN4 |
| 25 | SeSA_B0082 | | YP_002112961 | plasmid pCVM19633_110 |
| 26 | ESCAB7627_0644 | | ZP_02900823 | |
| 27 | STM474_p303 | repA | ADX20580 | plasmid TY474p3 |
| 28 | SPC_p013 | rsdB | YP_002635587 | plasmid pSPCV |
| 29 | SSO_P190 | traD | YP_313445 | plasmid pSS_046 |
| 30 | CP0245 | mvpA | NP_858378 | plasmid pCP301 |
| 31 | PSLT105 | trbH | NP_490589 | plasmid pSLT |
| 32 | STM14_5622 | traD | ACY86531 | |
| 33 | ESCAB7627_0650 | | ZP_02900555 | |
| 34 | SPC_p042 | traX | YP_002635616 | plasmid pSPCV |
| 35 | SPC_2707 | | YP_002638248 | |
| 36 | STM474_1038 | | ADX16744 | |
| 37 | Senterienterica_010100019467 | | ZP_02668762 | |
| 38 | SPC_p041 | finO | YP_002635615 | plasmid pSPCV |
| 39 | SPC_2708 | | YP_002638249 | |
| 40 | STM1046 | | NP_460021 | |
| 41 | STM474_p1093 | ccdB | ADX20463 | plasmid TY474p1 |
| 42 | Sentent_010100022581 | | ZP_02341659 | |
| 43 | SPAB_05276 | | YP_001591385 | |
| 44 | STMDT12_C10780 | | BAJ36021 | |
| 45 | SARI_01515 | | YP_001570553 | |
| 46 | SARI_01514 | | YP_001570552 | |
| 47 | SBG_1292 | malR | YP_004730162 | |
| 48 | Senterienterica_010100019192 | | ZP_02668707 | |
| 49 | SPC_0340 | | YP_002635966 | |
| 50 | Senteenterica_010100025269 | | ZP_02664569 | |

TABLE 18e
Data for Salmonella species (continued)

| No. | Start.Coord | End.Coord | Strand | Scaffold.ID |
|---|---|---|---|---|
| 1 | 1525960 | 1526820 | + | 642555218 |
| 2 | 68933 | 70240 | + | 642555219 |
| 3 | 3248 | 3841 | + | 648291919 |
| 4 | 34908 | 35255 | − | 640753021 |
| 5 | 32101 | 32916 | − | 646311959 |
| 6 | 36757 | 37062 | + | 641778223 |
| 7 | 48658 | 49503 | + | 643692014 |
| 8 | 4715 | 5554 | − | 648294624 |
| 9 | 31237 | 32040 | − | 646311959 |
| 10 | 3555 | 4568 | + | 638359041 |
| 11 | 1555 | 2055 | − | 651333542 |
| 12 | 8819 | 9535 | + | 648288593 |
| 13 | 1108351 | 1108854 | − | 646862363 |
| 14 | 4110458 | 4111246 | − | 640427102 |
| 15 | 97 | 531 | − | 648293053 |
| 16 | 19257 | 19691 | + | 641778223 |
| 17 | 23310 | 24614 | − | 640753020 |
| 18 | 16271 | 16636 | − | 641778223 |
| 19 | 1071 | 1307 | − | 648294596 |
| 20 | 812 | 1663 | − | 648294425 |
| 21 | 1107342 | 1107968 | + | 646862363 |
| 22 | 16416 | 17195 | + | 642555216 |
| 23 | 4088051 | 4088326 | − | 651053007 |
| 24 | 25970 | 27664 | − | 640753020 |
| 25 | 71061 | 71411 | − | 642555219 |
| 26 | 233492 | 234697 | − | 641784575 |
| 27 | 1988 | 2827 | − | 650378006 |
| 28 | 10060 | 10842 | − | 643692029 |
| 29 | 166425 | 168626 | + | 640427103 |
| 30 | 198117 | 198515 | − | 637000224 |
| 31 | 86566 | 87285 | + | 637000134 |
| 32 | 84297 | 86459 | + | 646862361 |
| 33 | 238788 | 239996 | − | 641784575 |
| 34 | 28413 | 29153 | − | 643692029 |
| 35 | 2745099 | 2745596 | − | 643692030 |
| 36 | 1088568 | 1089272 | + | 650378003 |
| 37 | 62341 | 62874 | − | 641777794 |
| 38 | 27795 | 28358 | − | 643692029 |
| 39 | 2745695 | 2746027 | − | 643692030 |
| 40 | 1131381 | 1132118 | + | 637000122 |
| 41 | 73221 | 73526 | − | 650378004 |
| 42 | 23575 | 23910 | − | 641746440 |
| 43 | 4445787 | 4448009 | + | 641228523 |
| 44 | 1141208 | 1141399 | − | 651053007 |
| 45 | 1476854 | 1478026 | + | 641228515 |
| 46 | 1475252 | 1476844 | + | 641228515 |
| 47 | 1414087 | 1415115 | + | 650716177 |
| 48 | 26832 | 28046 | + | 641777794 |
| 49 | 367421 | 368839 | + | 643692030 |
| 50 | 35858 | 36535 | − | 641777693 |

### Example 10

### Serratia, particularly Serratia marcescens

The procedure was carried out as in Example 1, except that the following microorganisms were used:

Bacterial Strains

The inventors selected 437 Serratia strains from the microbiology strain collection at Siemens Healthcare Diagnostics (West Sacramento, Calif.) for susceptibility testing and whole genome sequencing.

From MetaRef, 1671 centroids of Serratia species were used as reference sequences.

The results for Serratia are shown in Tables 19 (corresponding to Table 1) and 20 (corresponding to Table 2).

TABLE 19a
Data for Serratia, particularly Serratia marcescens

| | Scaffold.External. | | |
|---|---|---|---|
| No. | Accession | Scaffold.Name | best_pv |
| 1 | NC_009832 | Serratia proteamaculans 568: NC_009832 | 3.70422109249874e−06 |
| 2 | NZ_ADBY01000058 | Serratia odorifera DSM 4582 contig00061: NZ_ADBY01000058 | 6.00624466630552e−06 |
| 3 | NZ_ADBY01000056 | Serratia odorifera DSM 4582 contig00059: NZ_ADBY01000056 | 6.00624466630552e−06 |
| 4 | NZ_ADBY01000031 | Serratia odorifera DSM 4582 contig00034: NZ_ADBY01000031 | 8.61505367610747e−06 |
| 5 | NZ_GL636422 | Serratia symbiotica str. Tucson genomic scaffold contig09350: | 8.61505367610747e−06 |
| | | NZ_GL636422 | |
| 6 | NZ_ADBX01000013 | Serratia odorifera 4Rx13 SODm: NZ_ADBX01000013 | 8.61505367610747e−06 |
| 7 | NZ_ADBX01000004 | Serratia odorifera 4Rx13 SODd: NZ_ADBX01000004 | 1.33354783010227e−05 |
| 8 | NC_009832 | Serratia proteamaculans 568: NC_009832 | 1.37889112422256e−05 |
| 9 | NZ_ADBX01000010 | Serratia odorifera 4Rx13 SODj: NZ_ADBX01000010 | 1.55998636550055e−05 |
| 10 | NZ_ADBX01000013 | Serratia odorifera 4Rx13 SODm: NZ_ADBX01000013 | 1.55998636550055e−05 |
| 11 | NZ_GL636105 | Serratia symbiotica str. Tucson genomic scaffold scaffold00209: | 1.55998636550055e−05 |
| | | NZ_GL636105 | |
| 12 | NC_014837 | Pantoea sp. At-9b chromosome: NC_014837 | 1.55998636550055e−05 |
| 13 | NZ_GL636364 | Serratia symbiotica str. Tucson genomic scaffold contig09039: | 1.55998636550055e−05 |
| | | NZ_GL636364 | |
| 14 | NC_009832 | Serratia proteamaculans 568: NC_009832 | 1.55998636550055e−05 |
| 15 | NZ_ADBY01000048 | Serratia odorifera DSM 4582 contig00051: NZ_ADBY01000048 | 2.17438866604963e−05 |
| 16 | NZ_ADBX01000002 | Serratia odorifera 4Rx13 SODb: NZ_ADBX01000002 | 2.94809945975299e−05 |
| 17 | NZ_ADBY01000031 | Serratia odorifera DSM 4582 contig00034: NZ_ADBY01000031 | 3.13750499443641e−05 |
| 18 | NZ_ADBY01000045 | Serratia odorifera DSM 4582 contig00048: NZ_ADBY01000045 | 3.13750499443641e−05 |
| 19 | NZ_ADBY01000031 | Serratia odorifera DSM 4582 contig00034: NZ_ADBY01000031 | 3.13750499443641e−05 |
| 20 | NZ_ADBY01000058 | Serratia odorifera DSM 4582 contig00061: NZ_ADBY01000058 | 3.13750499443641e−05 |
| 21 | NZ_ADBX01000002 | Serratia odorifera 4Rx13 SODb: NZ_ADBX01000002 | 3.43966250408489e−05 |
| 22 | NZ_ADBY01000050 | Serratia odorifera DSM 4582 contig00053: NZ_ADBY01000050 | 3.66332257629745e−05 |
| 23 | NZ_ADBY01000013 | Serratia odorifera DSM 4582 contig00016: NZ_ADBY01000013 | 3.7147650177074e−05 |
| 24 | NZ_ADBY01000056 | Serratia odorifera DSM 4582 contig00059: NZ_ADBY01000056 | 3.7147650177074e−05 |
| 25 | NZ_ADBY01000026 | Serratia odorifera DSM 4582 contig00029: NZ_ADBY01000026 | 3.7147650177074e−05 |
| 26 | NZ_ADBX01000001 | Serratia odorifera 4Rx13 SODa: NZ_ADBX01000001 | 3.77977323028027e−05 |
| 27 | NZ_ADBY01000057 | Serratia odorifera DSM 4582 contig00060: NZ_ADBY01000057 | 4.06693999812307e−05 |
| 28 | NC_009832 | Serratia proteamaculans 568: NC_009832 | 4.06693999812307e−05 |
| 29 | NZ_ADBY01000057 | Serratia odorifera DSM 4582 contig00060: NZ_ADBY01000057 | 4.06693999812307e−05 |
| 30 | NZ_ADBX01000013 | Serratia odorifera 4Rx13 SODm: NZ_ADBX01000013 | 4.06693999812307e−05 |
| 31 | NZ_ADBY01000031 | Serratia odorifera DSM 4582 contig00034: NZ_ADBY01000031 | 4.20000578456372e−05 |
| 32 | NZ_ADBX01000002 | Serratia odorifera 4Rx13 SODb: NZ_ADBX01000002 | 5.02288669357547e−05 |
| 33 | NZ_ADBX01000002 | Serratia odorifera 4Rx13 SODb: NZ_ADBX01000002 | 5.02288669357547e−05 |
| 34 | NZ_ADBX01000003 | Serratia odorifera 4Rx13 SODc: NZ_ADBX01000003 | 5.02288669357547e−05 |
| 35 | NC_009832 | Serratia proteamaculans 568: NC_009832 | 5.02288669357547e−05 |
| 36 | NC_009832 | Serratia proteamaculans 568: NC_009832 | 5.02288669357547e−05 |
| 37 | NC_009832 | Serratia proteamaculans 568: NC_009832 | 5.02288669357547e−05 |
| 38 | NZ_ADBX01000004 | Serratia odorifera 4Rx13 SODd: NZ_ADBX01000004 | 5.02288669357547e−05 |
| 39 | NZ_GG657374 | Citrobacter sp. 30_2 genomic scaffold supercont1.9: NZ_GG657374 | 5.02288669357547e−05 |
| 40 | NC_009832 | Serratia proteamaculans 568: NC_009832 | 5.02288669357547e−05 |
| 41 | NC_009832 | Serratia proteamaculans 568: NC_009832 | 5.02288669357547e−05 |
| 42 | NC_009832 | Serratia proteamaculans 568: NC_009832 | 5.02288669357547e−05 |
| 43 | NZ_ADBX01000001 | Serratia odorifera 4Rx13 SODa: NZ_ADBX01000001 | 5.02288669357547e−05 |
| 44 | NZ_ADBX01000004 | Serratia odorifera 4Rx13 SODd: NZ_ADBX01000004 | 5.02288669357547e−05 |
| 45 | NZ_ADBX01000002 | Serratia odorifera 4Rx13 SODb: NZ_ADBX01000002 | 5.02288669357547e−05 |
| 46 | NZ_ADBY01000056 | Serratia odorifera DSM 4582 contig00059: NZ_ADBY01000056 | 5.02288669357547e−05 |
| 47 | NZ_ADBY01000032 | Serratia odorifera DSM 4582 contig00035: NZ_ADBY01000032 | 5.02288669357547e−05 |
| 48 | NZ_ADBY01000013 | Serratia odorifera DSM 4582 contig00016: NZ_ADBY01000013 | 5.88044402302405e−05 |
| 49 | NC_009832 | Serratia proteamaculans 568: NC_009832 | 6.62251962114689e−05 |
| 50 | NC_009832 | Serratia proteamaculans 568: NC_009832 | 7.15986854143193e−05 |

TABLE 19b
Data for Serratia, particularly Serratia marcescens (continued)

| No. | sign_phenos | sign_phenos_class | best_pheno | best_pheno_class |
|---|---|---|---|---|
| 1 | AZT; CAX; CAZ; CFT; CP; GM; LVX; P/T; TO; T/S | aminoglycoside; fluoroquinolone; lactam; other | LVX | fluoroquinolone |
| 2 | CP; LVX; T/S | fluoroquinolone; other | CP | fluoroquinolone |
| 3 | AZT; CAX; CP; LVX; T/S | fluoroquinolone; lactam; other | LVX | fluoroquinolone |
| 4 | AZT; CAX; CFT; CP; LVX; P/T; T/S | fluoroquinolone; lactam; other | LVX | fluoroquinolone |
| 5 | CAX; CAZ; CFT; CP; LVX; P/T | fluoroquinolone; lactam | CP | fluoroquinolone |
| 6 | AZT; CAX; CAZ; CFT; CP; LVX; P/T | fluoroquinolone; lactam | CP | fluoroquinolone |
| 7 | CAX; CFT; CP; LVX | fluoroquinolone; lactam | LVX | fluoroquinolone |
| 8 | CAX; CFT; CP; LVX; T/S | fluoroquinolone; lactam; other | LVX | fluoroquinolone |
| 9 | CAX; CFT; CP; LVX; TO | aminoglycoside; fluoroquinolone; lactam | LVX | fluoroquinolone |
| 10 | AZT; CAX; CAZ; CFT; CP; LVX; P/T | fluoroquinolone; lactam | CP | fluoroquinolone |
| 11 | AZT; CAX; CAZ; CFT; CP; P/T | fluoroquinolone; lactam | CAX | lactam |
| 12 | CAX; CFT; CP; GM; LVX; P/T; TO; T/S | aminoglycoside; fluoroquinolone; lactam; other | LVX | fluoroquinolone |
| 13 | AZT; CAX; CAZ; CFT; CP; P/T | fluoroquinolone; lactam | CAX | lactam |
| 14 | CP; LVX | fluoroquinolone | LVX | fluoroquinolone |
| 15 | AZT; CAX; CFT; CP; GM; LVX; P/T; TO; T/S | aminoglycoside; fluoroquinolone; lactam; other | LVX | fluoroquinolone |
| 16 | AZT; CAX; CFT; CP; GM; LVX; P/T; TO; T/S | aminoglycoside; fluoroquinolone; lactam; other | LVX | fluoroquinolone |
| 17 | AZT; CAX; CFT; CP; LVX; P/T; T/S | fluoroquinolone; lactam; other | LVX | fluoroquinolone |
| 18 | CAX; CFT; CP; GM; LVX; P/T; TO; T/S | aminoglycoside; fluoroquinolone; lactam; other | LVX | fluoroquinolone |
| 19 | AZT; CAX; CFT; CP; LVX; P/T; T/S | fluoroquinolone; lactam; other | LVX | fluoroquinolone |
| 20 | AZT; CAX; CFT; CP; GM; LVX; P/T; TO; T/S | aminoglycoside; fluoroquinolone; lactam; other | LVX | fluoroquinolone |
| 21 | CAX; CP; LVX; T/S | fluoroquinolone; lactam; other | LVX | fluoroquinolone |
| 22 | AZT; CAX; CFT; CP; GM; LVX; P/T; TO; T/S | aminoglycoside; fluoroquinolone; lactam; other | LVX | fluoroquinolone |
| 23 | CAX; CFT; CP; GM; LVX; P/T; TO; T/S | aminoglycoside; fluoroquinolone; lactam; other | CP | fluoroquinolone |
| 24 | AZT; CAX; CAZ; CFT; P/T | lactam | CAZ | lactam |
| 25 | CAX; CFT; CP; GM; LVX; P/T | aminoglycoside; fluoroquinolone; lactam | LVX | fluoroquinolone |
| 26 | AZT; CAX; CFT; CP; GM; LVX; P/T; TO; T/S | aminoglycoside; fluoroquinolone; lactam; other | LVX | fluoroquinolone |
| 27 | AZT; CAZ; CFT; CP; TO | aminoglycoside; fluoroquinolone; lactam | CP | fluoroquinolone |
| 28 | CAX; CP; LVX | fluoroquinolone; lactam | LVX | fluoroquinolone |
| 29 | AZT; CAZ; CFT; CP; TO | aminoglycoside; fluoroquinolone; lactam | CP | fluoroquinolone |
| 30 | AZT; CAZ; CFT; CP; TO | aminoglycoside; fluoroquinolone; lactam | CP | fluoroquinolone |
| 31 | CAX; CFT; CP; LVX; P/T; T/S | fluoroquinolone; lactam; other | P/T | lactam |
| 32 | AZT; CAX; CFT; CP; GM; LVX; P/T; TO; T/S | aminoglycoside; fluoroquinolone; lactam; other | LVX | fluoroquinolone |
| 33 | CAX; CFT; CP; LVX; T/S | fluoroquinolone; lactam; other | LVX | fluoroquinolone |
| 34 | CAX; CFT; CP; LVX; T/S | fluoroquinolone; lactam; other | LVX | fluoroquinolone |
| 35 | AZT; CAX; CFT; CP; GM; LVX; P/T; TO; T/S | aminoglycoside; fluoroquinolone; lactam; other | LVX | fluoroquinolone |
| 36 | AZT; CAX; CFT; CP; LVX; T/S | fluoroquinolone; lactam; other | CP | fluoroquinolone |
| 37 | AZT; CAX; CFT; CP; GM; LVX; P/T; TO; T/S | aminoglycoside; fluoroquinolone; lactam; other | LVX | fluoroquinolone |
| 38 | CAX; CFT; CP; LVX; P/T; T/S | fluoroquinolone; lactam; other | LVX | fluoroquinolone |
| 39 | CP; GM; LVX; TO; T/S | aminoglycoside; fluoroquinolone; other | LVX | fluoroquinolone |
| 40 | CAX; CFT; CP; LVX; P/T; T/S | fluoroquinolone; lactam; other | LVX | fluoroquinolone |
| 41 | AZT; CAX; CFT; CP; GM; LVX; P/T; TO; T/S | aminoglycoside; fluoroquinolone; lactam; other | LVX | fluoroquinolone |
| 42 | AZT; CAX; CFT; CP; GM; LVX; P/T; TO; T/S | aminoglycoside; fluoroquinolone; lactam; other | LVX | fluoroquinolone |
| 43 | CP; LVX; P/T | fluoroquinolone; lactam | LVX | fluoroquinolone |
| 44 | CAX; CFT; CP; LVX; T/S | fluoroquinolone; lactam; other | LVX | fluoroquinolone |
| 45 | AZT; CAX; CFT; CP; GM; LVX; P/T; TO; T/S | aminoglycoside; fluoroquinolone; lactam; other | LVX | fluoroquinolone |
| 46 | AZT; CAX; CAZ | lactam | CAZ | lactam |
| 47 | AZT; CAX; CFT; CP; GM; LVX; TO; T/S | aminoglycoside; fluoroquinolone; lactam; other | LVX | fluoroquinolone |
| 48 | CAX; CFT; CP; LVX; P/T | fluoroquinolone; lactam | LVX | fluoroquinolone |
| 49 | CAX; CFT; CP; LVX | fluoroquinolone; lactam | LVX | fluoroquinolone |
| 50 | AZT; CAX; CFT; CP; GM; LVX; P/T; TO; T/S | aminoglycoside; fluoroquinolone; lactam; other | LVX | fluoroquinolone |

TABLE 19c
Data for Serratia, particularly Serratia marcescens (continued)

| No. | num_aminoglycoside | num_fluoroquinolone | num_lactam | num_other |
|---|---|---|---|---|
| 1 | 2 | 2 | 5 | 1 |
| 2 | 0 | 2 | 0 | 1 |
| 3 | 0 | 2 | 2 | 1 |
| 4 | 0 | 2 | 4 | 1 |
| 5 | 0 | 2 | 4 | 0 |
| 6 | 0 | 2 | 5 | 0 |
| 7 | 0 | 2 | 2 | 0 |
| 8 | 0 | 2 | 2 | 1 |
| 9 | 1 | 2 | 2 | 0 |
| 10 | 0 | 2 | 5 | 0 |
| 11 | 0 | 1 | 5 | 0 |
| 12 | 2 | 2 | 3 | 1 |
| 13 | 0 | 1 | 5 | 0 |
| 14 | 0 | 2 | 0 | 0 |
| 15 | 2 | 2 | 4 | 1 |
| 16 | 2 | 2 | 4 | 1 |
| 17 | 0 | 2 | 4 | 1 |
| 18 | 2 | 2 | 3 | 1 |
| 19 | 0 | 2 | 4 | 1 |
| 20 | 2 | 2 | 4 | 1 |
| 21 | 0 | 2 | 1 | 1 |
| 22 | 2 | 2 | 4 | 1 |
| 23 | 2 | 2 | 3 | 1 |
| 24 | 0 | 0 | 5 | 0 |
| 25 | 1 | 2 | 3 | 0 |
| 26 | 2 | 2 | 4 | 1 |
| 27 | 1 | 1 | 3 | 0 |
| 28 | 0 | 2 | 1 | 0 |
| 29 | 1 | 1 | 3 | 0 |
| 30 | 1 | 1 | 3 | 0 |
| 31 | 0 | 2 | 3 | 1 |
| 32 | 2 | 2 | 4 | 1 |
| 33 | 0 | 2 | 2 | 1 |
| 34 | 0 | 2 | 2 | 1 |
| 35 | 2 | 2 | 4 | 1 |
| 36 | 0 | 2 | 3 | 1 |
| 37 | 2 | 2 | 4 | 1 |
| 38 | 0 | 2 | 3 | 1 |
| 39 | 2 | 2 | 0 | 1 |
| 40 | 0 | 2 | 3 | 1 |
| 41 | 2 | 2 | 4 | 1 |
| 42 | 2 | 2 | 4 | 1 |
| 43 | 0 | 2 | 1 | 0 |
| 44 | 0 | 2 | 2 | 1 |
| 45 | 2 | 2 | 4 | 1 |
| 46 | 0 | 0 | 3 | 0 |
| 47 | 2 | 2 | 3 | 1 |
| 48 | 0 | 2 | 3 | 0 |
| 49 | 0 | 2 | 2 | 0 |
| 50 | 2 | 2 | 4 | 1 |

TABLE 19d
Data for Serratia, particularly Serratia marcescens (continued)

| No. | AZT_pv_adj | CAX_pv_adj | CAZ_pv_adj | CFT_pv_adj | CP_pv_adj |
|---|---|---|---|---|---|
| 1 | 4.82E−04 | 5.24E−04 | 6.41E−03 | 7.29E−04 | 2.27E−05 |
| 2 | 1.58E−01 | 1.39E−01 | 2.26E−01 | 3.68E−02 | 6.01E−06 |
| 3 | 6.77E−04 | 8.30E−03 | 2.51E−02 | 1.65E−02 | 2.17E−05 |
| 4 | 8.92E−03 | 3.01E−03 | 3.16E−01 | 7.90E−03 | 2.95E−05 |
| 5 | 1.60E−02 | 4.77E−04 | 5.25E−03 | 1.36E−04 | 8.62E−06 |
| 6 | 1.22E−03 | 1.22E−03 | 1.24E−03 | 4.08E−04 | 8.62E−06 |
| 7 | 3.82E−02 | 3.55E−03 | 4.51E−02 | 2.17E−03 | 7.86E−05 |
| 8 | 1.48E−01 | 5.09E−03 | 6.93E−02 | 4.93E−03 | 1.10E−04 |
| 9 | 4.53E−02 | 5.02E−03 | 3.12E−01 | 2.64E−03 | 5.13E−04 |
| 10 | 6.43E−04 | 1.61E−04 | 7.67E−04 | 8.45E−05 | 1.56E−05 |
| 11 | 3.67E−05 | 1.56E−05 | 1.56E−05 | 5.02E−05 | 6.54E−03 |
| 12 | 1.44E−02 | 6.36E−03 | 4.83E−02 | 3.41E−03 | 2.36E−04 |
| 13 | 3.67E−05 | 1.56E−05 | 1.56E−05 | 5.02E−05 | 6.54E−03 |
| 14 | 4.07E−01 | 5.36E−02 | 7.62E−01 | 4.93E−02 | 5.86E−04 |
| 15 | 1.11E−03 | 7.67E−04 | 4.28E−02 | 1.04E−03 | 4.94E−05 |
| 16 | 6.43E−04 | 1.24E−03 | 2.38E−02 | 2.68E−03 | 1.82E−04 |
| 17 | 4.26E−03 | 1.84E−03 | 6.64E−02 | 4.93E−03 | 1.22E−04 |
| 18 | 1.73E−02 | 1.76E−04 | 2.04E−02 | 5.24E−04 | 6.30E−04 |
| 19 | 4.26E−03 | 1.84E−03 | 6.64E−02 | 4.93E−03 | 1.22E−04 |
| 20 | 9.20E−04 | 1.82E−04 | 2.34E−02 | 1.48E−04 | 1.69E−04 |
| 21 | 2.19E−02 | 6.70E−03 | 1 | 2.46E−02 | 7.82E−04 |
| 22 | 1.55E−03 | 4.30E−04 | 6.08E−02 | 5.90E−04 | 1.52E−04 |
| 23 | 4.85E−02 | 7.20E−04 | 1.01E−01 | 6.90E−04 | 3.71E−05 |
| 24 | 2.28E−03 | 2.41E−03 | 3.71E−05 | 4.23E−03 | 1.66E−01 |
| 25 | 1.01E−02 | 3.27E−04 | 5.44E−02 | 4.95E−04 | 7.62E−04 |
| 26 | 8.18E−04 | 3.20E−04 | 4.45E−02 | 3.27E−04 | 1.53E−04 |
| 27 | 5.89E−04 | 1.07E−02 | 1.19E−03 | 3.35E−03 | 4.07E−05 |
| 28 | 2.82E−02 | 6.08E−03 | 2.88E−01 | 4.51E−02 | 1.10E−04 |
| 29 | 5.89E−04 | 1.07E−02 | 1.19E−03 | 3.35E−03 | 4.07E−05 |
| 30 | 5.89E−04 | 1.07E−02 | 1.19E−03 | 3.35E−03 | 4.07E−05 |
| 31 | 5.92E−02 | 3.15E−04 | 6.50E−01 | 1.11E−03 | 6.89E−03 |
| 32 | 1.66E−03 | 6.30E−04 | 8.28E−02 | 6.43E−04 | 3.33E−04 |
| 33 | 5.14E−02 | 1.60E−03 | 5.77E−02 | 2.64E−03 | 1.17E−03 |
| 34 | 5.14E−02 | 1.60E−03 | 5.77E−02 | 2.64E−03 | 1.17E−03 |
| 35 | 1.17E−03 | 3.15E−04 | 5.90E−02 | 3.27E−04 | 3.15E−04 |
| 36 | 1.07E−03 | 3.99E−03 | 2.05E−02 | 2.84E−03 | 5.02E−05 |
| 37 | 1.16E−03 | 5.89E−04 | 5.88E−02 | 6.07E−04 | 2.32E−04 |
| 38 | 4.11E−02 | 1.55E−03 | 5.76E−02 | 2.12E−03 | 1.15E−03 |
| 39 | 2.08E−02 | 2.04E−02 | 2.72E−01 | 3.09E−02 | 3.33E−04 |
| 40 | 5.14E−02 | 1.13E−03 | 5.80E−02 | 1.53E−03 | 1.50E−03 |
| 41 | 1.17E−03 | 3.15E−04 | 5.90E−02 | 3.27E−04 | 3.15E−04 |
| 42 | 4.30E−03 | 1.43E−03 | 1.74E−01 | 2.47E−03 | 9.38E−04 |
| 43 | 8.63E−02 | 1.24E−02 | 2.31E−01 | 1.12E−02 | 7.34E−04 |
| 44 | 5.14E−02 | 2.07E−03 | 5.80E−02 | 2.73E−03 | 1.50E−03 |
| 45 | 1.17E−03 | 3.15E−04 | 5.90E−02 | 3.27E−04 | 3.15E−04 |
| 46 | 2.22E−03 | 6.95E−03 | 5.02E−05 | 1.63E−02 | 4.21E−01 |
| 47 | 7.04E−03 | 6.21E−03 | 3.82E−02 | 3.31E−03 | 1.04E−04 |
| 48 | 3.16E−01 | 1.58E−03 | 5.58E−01 | 1.57E−03 | 7.78E−04 |
| 49 | 2.46E−02 | 8.70E−03 | 2.31E−01 | 5.07E−03 | 1.80E−03 |
| 50 | 1.55E−03 | 7.82E−04 | 5.97E−02 | 8.10E−04 | 3.15E−04 |

TABLE 19e
Data for Serratia, particularly Serratia marcescens (continued)

| No. | GM_pv_adj | LVX_pv_adj | P/T_pv_adj | TO_pv_adj | T/S_pv_adj |
|---|---|---|---|---|---|
| 1 | 7.29E−04 | 3.70E−06 | 4.19E−03 | 5.65E−04 | 2.79E−04 |
| 2 | 3.92E−02 | 8.62E−06 | 5.32E−01 | 5.08E−02 | 9.68E−03 |
| 3 | 3.60E−02 | 6.01E−06 | 4.96E−02 | 9.86E−02 | 1.55E−03 |
| 4 | 2.38E−01 | 8.62E−06 | 4.51E−04 | 1.33E−01 | 5.10E−03 |
| 5 | 2.87E−01 | 4.05E−04 | 5.63E−05 | 3.66E−02 | 2.11E−02 |
| 6 | 9.01E−01 | 8.10E−04 | 6.41E−03 | 4.07E−02 | 1.63E−01 |
| 7 | 3.66E−02 | 1.33E−05 | 1.49E−02 | 1.15E−02 | 2.81E−02 |
| 8 | 1.16E−01 | 1.38E−05 | 1.31E−02 | 6.43E−02 | 5.52E−03 |
| 9 | 1.25E−02 | 1.56E−05 | 5.88E−02 | 1.37E−03 | 3.00E−02 |
| 10 | 2.58E−01 | 1.22E−04 | 9.05E−05 | 4.31E−02 | 3.97E−02 |
| 11 | 2.06E−01 | 3.60E−01 | 3.67E−05 | 1.87E−01 | 3.72E−01 |
| 12 | 5.33E−03 | 1.56E−05 | 6.87E−03 | 3.46E−03 | 8.20E−03 |
| 13 | 2.06E−01 | 3.60E−01 | 3.67E−05 | 1.87E−01 | 3.72E−01 |
| 14 | 4.95E−02 | 1.56E−05 | 1.34E−01 | 1.43E−01 | 2.07E−02 |
| 15 | 2.56E−03 | 2.17E−05 | 2.35E−03 | 3.96E−03 | 4.94E−04 |
| 16 | 3.89E−04 | 2.95E−05 | 1.52E−03 | 3.51E−03 | 2.43E−03 |
| 17 | 1.48E−01 | 3.14E−05 | 2.32E−04 | 8.78E−02 | 5.89E−04 |
| 18 | 3.24E−03 | 3.14E−05 | 2.41E−03 | 4.32E−03 | 4.63E−03 |
| 19 | 1.48E−01 | 3.14E−05 | 2.32E−04 | 8.78E−02 | 5.89E−04 |
| 20 | 2.78E−03 | 3.14E−05 | 1.10E−03 | 2.42E−03 | 6.75E−04 |
| 21 | 2.77E−02 | 3.44E−05 | 5.49E−02 | 1.12E−01 | 1.40E−03 |
| 22 | 5.63E−03 | 3.66E−05 | 3.16E−03 | 5.49E−03 | 3.24E−03 |
| 23 | 3.72E−03 | 4.07E−05 | 1.76E−03 | 4.82E−03 | 3.60E−03 |
| 24 | 2.22E−01 | 1 | 9.74E−03 | 1.18E−01 | 2.81E−01 |
| 25 | 4.82E−03 | 3.71E−05 | 7.82E−04 | 2.06E−02 | 1.08E−02 |
| 26 | 1.24E−03 | 3.78E−05 | 1.36E−03 | 2.34E−03 | 1.55E−03 |
| 27 | 6.23E−01 | 1.02E−02 | 5.43E−02 | 8.38E−03 | 8.82E−02 |
| 28 | 5.36E−02 | 4.07E−05 | 4.63E−02 | 3.92E−02 | 1.38E−02 |
| 29 | 6.23E−01 | 1.02E−02 | 5.43E−02 | 8.38E−03 | 8.82E−02 |
| 30 | 6.23E−01 | 1.02E−02 | 5.43E−02 | 8.38E−03 | 8.82E−02 |
| 31 | 1.41E−01 | 7.82E−04 | 4.20E−05 | 3.04E−02 | 5.10E−03 |
| 32 | 2.77E−03 | 5.02E−05 | 1.37E−03 | 5.69E−03 | 2.21E−03 |
| 33 | 1.47E−02 | 5.02E−05 | 1.03E−02 | 3.27E−02 | 6.75E−03 |
| 34 | 1.47E−02 | 5.02E−05 | 1.03E−02 | 3.27E−02 | 6.75E−03 |
| 35 | 1.76E−03 | 5.02E−05 | 1.07E−03 | 4.09E−03 | 2.26E−03 |
| 36 | 2.27E−02 | 3.89E−04 | 4.16E−02 | 1.11E−02 | 6.71E−03 |
| 37 | 1.76E−03 | 5.02E−05 | 1.80E−03 | 3.17E−03 | 2.24E−03 |
| 38 | 1.48E−02 | 5.02E−05 | 8.05E−03 | 2.57E−02 | 6.70E−03 |
| 39 | 2.77E−03 | 5.02E−05 | 1.42E−02 | 2.41E−03 | 2.10E−04 |
| 40 | 1.48E−02 | 5.02E−05 | 5.52E−03 | 3.27E−02 | 6.89E−03 |
| 41 | 1.76E−03 | 5.02E−05 | 1.07E−03 | 4.09E−03 | 2.26E−03 |
| 42 | 1.31E−03 | 5.02E−05 | 5.93E−03 | 3.06E−03 | 2.21E−03 |
| 43 | 3.13E−02 | 5.02E−05 | 5.52E−03 | 1.02E−02 | 9.79E−02 |
| 44 | 1.48E−02 | 5.02E−05 | 1.06E−02 | 3.27E−02 | 6.89E−03 |
| 45 | 1.76E−03 | 5.02E−05 | 1.07E−03 | 4.09E−03 | 2.26E−03 |
| 46 | 2.06E−01 | 1 | 7.15E−02 | 3.33E−01 | 1.57E−01 |
| 47 | 9.28E−04 | 5.02E−05 | 6.47E−02 | 2.47E−03 | 1.76E−03 |
| 48 | 1.83E−02 | 5.88E−05 | 1.62E−03 | 1.34E−02 | 1.37E−02 |
| 49 | 9.13E−01 | 6.62E−05 | 6.12E−02 | 1.22E−01 | 1.54E−01 |
| 50 | 1.80E−03 | 7.16E−05 | 2.42E−03 | 4.20E−03 | 2.34E−03 |

TABLE 19f
Data for Serratia, particularly Serratia marcescens (continued)

| No. | Locus.Tag | Gene.Symbol | GenBank.Accession | Chromosome |
|---|---|---|---|---|
| 1 | Spro_0657 | | YP_001476891 | |
| 2 | HMPREF0758_4528 | | ZP_06641192 | |
| 3 | HMPREF0758_4282 | wrbA | ZP_06640946 | |
| 4 | HMPREF0758_1761 | | ZP_06638425 | |
| 5 | SSYM_0272 | | ZP_08040542 | |
| 6 | SOD_m00050 | | ZP_06193534 | |
| 7 | SOD_d02380 | | ZP_06191491 | |
| 8 | Spro_3910 | | YP_001480133 | |
| 9 | SOD_j00160 | | ZP_06193066 | |
| 10 | SOD_m00140 | | ZP_06193543 | |
| 11 | SSYM_0779 | | ZP_08038782 | |
| 12 | Pat9b_2844 | | YP_004116700 | |
| 13 | SSYM_0215 | | ZP_08040491 | |
| 14 | Spro_1286 | | YP_001477518 | |
| 15 | HMPREF0758_3087 | paaY | ZP_06639751 | |
| 16 | SOD_b04490 | | ZP_06190513 | |
| 17 | HMPREF0758_1765 | | ZP_06638429 | |
| 18 | HMPREF0758_2630 | deoR | ZP_06639294 | |
| 19 | HMPREF0758_1762 | vtaK | ZP_06638426 | |
| 20 | HMPREF0758_4768 | yedI | ZP_06641432 | |
| 21 | SOD_b01290 | | ZP_06190194 | |
| 22 | HMPREF0758_3449 | mdtJ | ZP_06640113 | |
| 23 | HMPREF0758_0340 | | ZP_06637004 | |
| 24 | HMPREF0758_4122 | dnaC | ZP_06640786 | |
| 25 | HMPREF0758_1641 | pepE | ZP_06638305 | |
| 26 | SOD_a01780 | | ZP_06189226 | |
| 27 | HMPREF0758_4410 | | ZP_06641074 | |
| 28 | Spro_0998 | | YP_001477230 | |
| 29 | HMPREF0758_4409 | | ZP_06641073 | |
| 30 | SOD_m00010 | | ZP_06193530 | |
| 31 | HMPREF0758_1747 | | ZP_06638411 | |
| 32 | SOD_b01600 | | ZP_06190225 | |
| 33 | SOD_b01460 | | ZP_06190211 | |
| 34 | SOD_c02470 | | ZP_06190898 | |
| 35 | Spro_2856 | | YP_001479085 | |
| 36 | Spro_1467 | | YP_001477699 | |
| 37 | Spro_1011 | | YP_001477243 | |
| 38 | SOD_d01640 | | ZP_06191418 | |
| 39 | CSAG_04704 | | ZP_04558356 | |
| 40 | Spro_0906 | | YP_001477140 | |
| 41 | Spro_2853 | | YP_001479082 | |
| 42 | Spro_4574 | | YP_001480795 | |
| 43 | SOD_a08590 | | ZP_06189897 | |
| 44 | SOD_d03490 | | ZP_06191602 | |
| 45 | SOD_b05060 | | ZP_06190570 | |
| 46 | HMPREF0758_4124 | cpaE | ZP_06640788 | |
| 47 | HMPREF0758_1879 | yhcC | ZP_06638543 | |
| 48 | HMPREF0758_0325 | apbE | ZP_06636989 | |
| 49 | Spro_2867 | | YP_001479096 | |
| 50 | Spro_0904 | | YP_001477138 | |

TABLE 19g
Data for Serratia, particularly Serratia marcescens (continued)

| No. | Start.Coord | End.Coord | Strand | Scaffold.ID |
|---|---|---|---|---|
| 1 | 721013 | 721786 | + | 640753091 |
| 2 | 113063 | 113683 | + | 647012891 |
| 3 | 196203 | 196802 | + | 647012889 |
| 4 | 14537 | 15286 | + | 647012864 |
| 5 | 1 | 622 | + | 651285392 |
| 6 | 5289 | 5756 | − | 647012829 |
| 7 | 276617 | 277270 | − | 647012820 |
| 8 | 4328564 | 4329223 | + | 640753091 |
| 9 | 10640 | 11029 | − | 647012826 |
| 10 | 12102 | 12680 | − | 647012829 |
| 11 | 54235 | 55095 | − | 651285075 |
| 12 | 3117915 | 3119003 | − | 649633106 |
| 13 | 1 | 203 | + | 651285334 |
| 14 | 1415512 | 1416153 | − | 640753091 |
| 15 | 166028 | 166624 | − | 647012881 |
| 16 | 563304 | 564224 | − | 647012818 |
| 17 | 17164 | 19063 | + | 647012864 |
| 18 | 1577 | 1921 | + | 647012878 |
| 19 | 15286 | 15990 | + | 647012864 |
| 20 | 357014 | 357931 | − | 647012891 |
| 21 | 207219 | 208373 | − | 647012818 |
| 22 | 144943 | 145311 | + | 647012883 |
| 23 | 46700 | 47314 | − | 647012846 |
| 24 | 40618 | 41358 | − | 647012889 |
| 25 | 66823 | 67548 | + | 647012859 |
| 26 | 183381 | 184292 | + | 647012817 |
| 27 | 12440 | 13084 | − | 647012890 |
| 28 | 1101888 | 1102271 | + | 640753091 |
| 29 | 11210 | 12427 | − | 647012890 |
| 30 | 189 | 1409 | − | 647012829 |
| 31 | 1316 | 1822 | + | 647012864 |
| 32 | 243135 | 244313 | − | 647012818 |
| 33 | 227360 | 228310 | + | 647012818 |
| 34 | 276786 | 277940 | − | 647012819 |
| 35 | 3134756 | 3135724 | − | 640753091 |
| 36 | 1599118 | 1600077 | + | 640753091 |
| 37 | 1112389 | 1113570 | − | 640753091 |
| 38 | 196893 | 197789 | − | 647012820 |
| 39 | 17332 | 17544 | − | 646206740 |
| 40 | 1005118 | 1006293 | + | 640753091 |
| 41 | 3131862 | 3132830 | − | 640753091 |
| 42 | 5048349 | 5049131 | − | 640753091 |
| 43 | 927700 | 928224 | + | 647012817 |
| 44 | 395510 | 396043 | + | 647012820 |
| 45 | 616091 | 617041 | − | 647012818 |
| 46 | 42335 | 42577 | − | 647012889 |
| 47 | 107941 | 108870 | + | 647012865 |
| 48 | 30480 | 31511 | − | 647012846 |
| 49 | 3146974 | 3147780 | − | 640753091 |
| 50 | 1003277 | 1004080 | + | 640753091 |

TABLE 20a
Data for Serratia, particularly Serratia marcescens

| | Scaffold.External. | | |
|---|---|---|---|
| No. | Accession | Scaffold.Name | best_pv |
| 1 | NC_009832 | Serratia proteamaculans 568: NC_009832 | 3.70422109249874e−06 |
| 2 | NZ_ADBY01000058 | Serratia odorifera DSM 4582 contig00061: NZ_ADBY01000058 | 6.00624466630552e−06 |
| 3 | NZ_ADBY01000056 | Serratia odorifera DSM 4582 contig00059: NZ_ADBY01000056 | 6.00624466630552e−06 |
| 4 | NZ_ADBY01000031 | Serratia odorifera DSM 4582 contig00034: NZ_ADBY01000031 | 8.61505367610747e−06 |
| 5 | NZ_GL636422 | Serratia symbiotica str. Tucson genomic scaffold contig09350: | 8.61505367610747e−06 |
| | | NZ_GL636422 | |
| 6 | NZ_ADBX01000013 | Serratia odorifera 4Rx13 SODm: NZ_ADBX01000013 | 8.61505367610747e−06 |
| 7 | NZ_ADBX01000004 | Serratia odorifera 4Rx13 SODd: NZ_ADBX01000004 | 1.33354783010227e−05 |
| 8 | NC_009832 | Serratia proteamaculans 568: NC_009832 | 1.37889112422256e−05 |
| 9 | NZ_ADBX01000010 | Serratia odorifera 4Rx13 SODj: NZ_ADBX01000010 | 1.55998636550055e−05 |
| 10 | NZ_ADBX01000013 | Serratia odorifera 4Rx13 SODm: NZ_ADBX01000013 | 1.55998636550055e−05 |
| 11 | NZ_GL636105 | Serratia symbiotica str. Tucson genomic scaffold scaffold00209: | 1.55998636550055e−05 |
| | | NZ_GL636105 | |
| 12 | NC_014837 | Pantoea sp. At-9b chromosome: NC_014837 | 1.55998636550055e−05 |
| 13 | NZ_GL636364 | Serratia symbiotica str. Tucson genomic scaffold contig09039: | 1.55998636550055e−05 |
| | | NZ_GL636364 | |
| 14 | NC_009832 | Serratia proteamaculans 568: NC_009832 | 1.55998636550055e−05 |
| 15 | NZ_ADBY01000048 | Serratia odorifera DSM 4582 contig00051: NZ_ADBY01000048 | 2.17438866604963e−05 |
| 16 | NZ_ADBX01000002 | Serratia odorifera 4Rx13 SODb: NZ_ADBX01000002 | 2.94809945975299e−05 |
| 17 | NZ_ADBY01000031 | Serratia odorifera DSM 4582 contig00034: NZ_ADBY01000031 | 3.13750499443641e−05 |
| 18 | NZ_ADBY01000045 | Serratia odorifera DSM 4582 contig00048: NZ_ADBY01000045 | 3.13750499443641e−05 |
| 19 | NZ_ADBY01000031 | Serratia odorifera DSM 4582 contig00034: NZ_ADBY01000031 | 3.13750499443641e−05 |
| 20 | NZ_ADBY01000058 | Serratia odorifera DSM 4582 contig00061: NZ_ADBY01000058 | 3.13750499443641e−05 |
| 21 | NZ_ADBX01000002 | Serratia odorifera 4Rx13 SODb: NZ_ADBX01000002 | 3.43966250408489e−05 |
| 22 | NZ_ADBY01000050 | Serratia odorifera DSM 4582 contig00053: NZ_ADBY01000050 | 3.66332257629745e−05 |
| 23 | NZ_ADBY01000013 | Serratia odorifera DSM 4582 contig00016: NZ_ADBY01000013 | 3.7147650177074e−05 |
| 24 | NZ_ADBY01000056 | Serratia odorifera DSM 4582 contig00059: NZ_ADBY01000056 | 3.7147650177074e−05 |
| 25 | NZ_ADBY01000026 | Serratia odorifera DSM 4582 contig00029: NZ_ADBY01000026 | 3.7147650177074e−05 |
| 26 | NZ_ADBX01000001 | Serratia odorifera 4Rx13 SODa: NZ_ADBX01000001 | 3.77977323028027e−05 |
| 27 | NC_009832 | Serratia proteamaculans 568: NC_009832 | 4.06693999812307e−05 |
| 28 | NZ_ADBY01000031 | Serratia odorifera DSM 4582 contig00034: NZ_ADBY01000031 | 4.20000578456372e−05 |
| 29 | NZ_ADBX01000002 | Serratia odorifera 4Rx13 SODb: NZ_ADBX01000002 | 5.02288669357547e−05 |
| 30 | NZ_ADBX01000002 | Serratia odorifera 4Rx13 SODb: NZ_ADBX01000002 | 5.02288669357547e−05 |
| 31 | NZ_ADBX01000003 | Serratia odorifera 4Rx13 SODc: NZ_ADBX01000003 | 5.02288669357547e−05 |
| 32 | NC_009832 | Serratia proteamaculans 568: NC_009832 | 5.02288669357547e−05 |
| 33 | NC_009832 | Serratia proteamaculans 568: NC_009832 | 5.02288669357547e−05 |
| 34 | NC_009832 | Serratia proteamaculans 568: NC_009832 | 5.02288669357547e−05 |
| 35 | NZ_ADBX01000004 | Serratia odorifera 4Rx13 SODd: NZ_ADBX01000004 | 5.02288669357547e−05 |
| 36 | NZ_GG657374 | Citrobacter sp. 30_2 genomic scaffold supercont1.9: NZ_GG657374 | 5.02288669357547e−05 |
| 37 | NC_009832 | Serratia proteamaculans 568: NC_009832 | 5.02288669357547e−05 |
| 38 | NC_009832 | Serratia proteamaculans 568: NC_009832 | 5.02288669357547e−05 |
| 39 | NC_009832 | Serratia proteamaculans 568: NC_009832 | 5.02288669357547e−05 |
| 40 | NZ_ADBX01000001 | Serratia odorifera 4Rx13 SODa: NZ_ADBX01000001 | 5.02288669357547e−05 |
| 41 | NZ_ADBX01000004 | Serratia odorifera 4Rx13 SODd: NZ_ADBX01000004 | 5.02288669357547e−05 |
| 42 | NZ_ADBX01000002 | Serratia odorifera 4Rx13 SODb: NZ_ADBX01000002 | 5.02288669357547e−05 |
| 43 | NZ_ADBY01000032 | Serratia odorifera DSM 4582 contig00035: NZ_ADBY01000032 | 5.02288669357547e−05 |
| 44 | NZ_ADBY01000013 | Serratia odorifera DSM 4582 contig00016: NZ_ADBY01000013 | 5.88044402302405e−05 |
| 45 | NC_009832 | Serratia proteamaculans 568: NC_009832 | 6.62251962114689e−05 |
| 46 | NC_009832 | Serratia proteamaculans 568: NC_009832 | 7.15986854143193e−05 |
| 47 | NZ_ADBX01000002 | Serratia odorifera 4Rx13 SODb: NZ_ADBX01000002 | 7.1811098690049e−05 |
| 48 | NC_009832 | Serratia proteamaculans 568: NC_009832 | 7.1811098690049e−05 |
| 49 | NC_009832 | Serratia proteamaculans 568: NC_009832 | 7.1811098690049e−05 |
| 50 | NZ_ADBY01000036 | Serratia odorifera DSM 4582 contig00039: NZ_ADBY01000036 | 7.1811098690049e−05 |

TABLE 20b
Data for Serratia, particularly Serratia marcescens (continued)

| No. | sign_phenos | sign_phenos_class | best_pheno | best_pheno_class |
|---|---|---|---|---|
| 1 | AZT; CAX; CAZ; CFT; CP; GM; LVX; P/T; TO; T/S | aminoglycoside; fluoroquinolone; lactam; other | LVX | fluoroquinolone |
| 2 | CP; LVX; T/S | fluoroquinolone; other | CP | fluoroquinolone |
| 3 | AZT; CAX; CP; LVX; T/S | fluoroquinolone; lactam; other | LVX | fluoroquinolone |
| 4 | AZT; CAX; CFT; CP; LVX; P/T; T/S | fluoroquinolone; lactam; other | LVX | fluoroquinolone |
| 5 | CAX; CAZ; CFT; CP; LVX; P/T | fluoroquinolone; lactam | CP | fluoroquinolone |
| 6 | AZT; CAX; CAZ; CFT; CP; LVX; P/T | fluoroquinolone; lactam | CP | fluoroquinolone |
| 7 | CAX; CFT; CP; LVX | fluoroquinolone; lactam | LVX | fluoroquinolone |
| 8 | CAX; CFT; CP; LVX; T/S | fluoroquinolone; lactam; other | LVX | fluoroquinolone |
| 9 | CAX; CFT; CP; LVX; TO | aminoglycoside; fluoroquinolone; lactam | LVX | fluoroquinolone |
| 10 | AZT; CAX; CAZ; CFT; CP; LVX; P/T | fluoroquinolone; lactam | CP | fluoroquinolone |
| 11 | AZT; CAX; CAZ; CFT; CP; P/T | fluoroquinolone; lactam | CAX | lactam |
| 12 | CAX; CFT; CP; GM; LVX; P/T; TO; T/S | aminoglycoside; fluoroquinolone; lactam; other | LVX | fluoroquinolone |
| 13 | AZT; CAX; CAZ; CFT; CP; P/T | fluoroquinolone; lactam | CAX | lactam |
| 14 | CP; LVX | fluoroquinolone | LVX | fluoroquinolone |
| 15 | AZT; CAX; CFT; CP; GM; LVX; P/T; TO; T/S | aminoglycoside; fluoroquinolone; lactam; other | LVX | fluoroquinolone |
| 16 | AZT; CAX; CFT; CP; GM; LVX; P/T; TO; T/S | aminoglycoside; fluoroquinolone; lactam; other | LVX | fluoroquinolone |
| 17 | AZT; CAX; CFT; CP; LVX; P/T; T/S | fluoroquinolone; lactam; other | LVX | fluoroquinolone |
| 18 | CAX; CFT; CP; GM; LVX; P/T; TO; T/S | aminoglycoside; fluoroquinolone; lactam; other | LVX | fluoroquinolone |
| 19 | AZT; CAX; CFT; CP; LVX; P/T; T/S | fluoroquinolone; lactam; other | LVX | fluoroquinolone |
| 20 | AZT; CAX; CFT; CP; GM; LVX; P/T; TO; T/S | aminoglycoside; fluoroquinolone; lactam; other | LVX | fluoroquinolone |
| 21 | CAX; CP; LVX; T/S | fluoroquinolone; lactam; other | LVX | fluoroquinolone |
| 22 | AZT; CAX; CFT; CP; GM; LVX; P/T; TO; T/S | aminoglycoside; fluoroquinolone; lactam; other | LVX | fluoroquinolone |
| 23 | CAX; CFT; CP; GM; LVX; P/T; TO; T/S | aminoglycoside; fluoroquinolone; lactam; other | CP | fluoroquinolone |
| 24 | AZT; CAX; CAZ; CFT; P/T | lactam | CAZ | lactam |
| 25 | CAX; CFT; CP; GM; LVX; P/T | aminoglycoside; fluoroquinolone; lactam | LVX | fluoroquinolone |
| 26 | AZT; CAX; CFT; CP; GM; LVX; P/T; TO; T/S | aminoglycoside; fluoroquinolone; lactam; other | LVX | fluoroquinolone |
| 27 | CAX; CP; LVX | fluoroquinolone; lactam | LVX | fluoroquinolone |
| 28 | CAX; CFT; CP; LVX; P/T; T/S | fluoroquinolone; lactam; other | P/T | lactam |
| 29 | AZT; CAX; CFT; CP; GM; LVX; P/T; TO; T/S | aminoglycoside; fluoroquinolone; lactam; other | LVX | fluoroquinolone |
| 30 | CAX; CFT; CP; LVX; T/S | fluoroquinolone; lactam; other | LVX | fluoroquinolone |
| 31 | CAX; CFT; CP; LVX; T/S | fluoroquinolone; lactam; other | LVX | fluoroquinolone |
| 32 | AZT; CAX; CFT; CP; GM; LVX; P/T; TO; T/S | aminoglycoside; fluoroquinolone; lactam; other | LVX | fluoroquinolone |
| 33 | AZT; CAX; CFT; CP; LVX; T/S | fluoroquinolone; lactam; other | CP | fluoroquinolone |
| 34 | AZT; CAX; CFT; CP; GM; LVX; P/T; TO; T/S | aminoglycoside; fluoroquinolone; lactam; other | LVX | fluoroquinolone |
| 35 | CAX; CFT; CP; LVX; P/T; T/S | fluoroquinolone; lactam; other | LVX | fluoroquinolone |
| 36 | CP; GM; LVX; TO; T/S | aminoglycoside; fluoroquinolone; other | LVX | fluoroquinolone |
| 37 | CAX; CFT; CP; LVX; P/T; T/S | fluoroquinolone; lactam; other | LVX | fluoroquinolone |
| 38 | AZT; CAX; CFT; CP; GM; LVX; P/T; TO; T/S | aminoglycoside; fluoroquinolone; lactam; other | LVX | fluoroquinolone |
| 39 | AZT; CAX; CFT; CP; GM; LVX; P/T; TO; T/S | aminoglycoside; fluoroquinolone; lactam; other | LVX | fluoroquinolone |
| 40 | CP; LVX; P/T | fluoroquinolone; lactam | LVX | fluoroquinolone |
| 41 | CAX; CFT; CP; LVX; T/S | fluoroquinolone; lactam; other | LVX | fluoroquinolone |
| 42 | AZT; CAX; CFT; CP; GM; LVX; P/T; TO; T/S | aminoglycoside; fluoroquinolone; lactam; other | LVX | fluoroquinolone |
| 43 | AZT; CAX; CFT; CP; GM; LVX; TO; T/S | aminoglycoside; fluoroquinolone; lactam; other | LVX | fluoroquinolone |
| 44 | CAX; CFT; CP; LVX; P/T | fluoroquinolone; lactam | LVX | fluoroquinolone |
| 45 | CAX; CFT; CP; LVX | fluoroquinolone; lactam | LVX | fluoroquinolone |
| 46 | AZT; CAX; CFT; CP; GM; LVX; P/T; TO; T/S | aminoglycoside; fluoroquinolone; lactam; other | LVX | fluoroquinolone |
| 47 | AZT; CAX; CFT; CP; LVX; P/T; TO | aminoglycoside; fluoroquinolone; lactam | CP | fluoroquinolone |
| 48 | CAX; CFT; CP; LVX; T/S | fluoroquinolone; lactam; other | LVX | fluoroquinolone |
| 49 | CFT; CP; LVX | fluoroquinolone; lactam | LVX | fluoroquinolone |
| 50 | AZT; CAX; CFT; CP; GM; LVX; P/T | aminoglycoside; fluoroquinolone; lactam | CAX | lactam |

TABLE 20c
Data for Serratia, particularly Serratia marcescens (continued)

| No. | num_aminoglycoside | num_fluoroquinolone | num_lactam | num_other |
|---|---|---|---|---|
| 1 | 2 | 2 | 5 | 1 |
| 2 | 0 | 2 | 0 | 1 |
| 3 | 0 | 2 | 2 | 1 |
| 4 | 0 | 2 | 4 | 1 |
| 5 | 0 | 2 | 4 | 0 |
| 6 | 0 | 2 | 5 | 0 |
| 7 | 0 | 2 | 2 | 0 |
| 8 | 0 | 2 | 2 | 1 |
| 9 | 1 | 2 | 2 | 0 |
| 10 | 0 | 2 | 5 | 0 |
| 11 | 0 | 1 | 5 | 0 |
| 12 | 2 | 2 | 3 | 1 |
| 13 | 0 | 1 | 5 | 0 |
| 14 | 0 | 2 | 0 | 0 |
| 15 | 2 | 2 | 4 | 1 |
| 16 | 2 | 2 | 4 | 1 |
| 17 | 0 | 2 | 4 | 1 |
| 18 | 2 | 2 | 3 | 1 |
| 19 | 0 | 2 | 4 | 1 |
| 20 | 2 | 2 | 4 | 1 |
| 21 | 0 | 2 | 1 | 1 |
| 22 | 2 | 2 | 4 | 1 |
| 23 | 2 | 2 | 3 | 1 |
| 24 | 0 | 0 | 5 | 0 |
| 25 | 1 | 2 | 3 | 0 |
| 26 | 2 | 2 | 4 | 1 |
| 27 | 0 | 2 | 1 | 0 |
| 28 | 0 | 2 | 3 | 1 |
| 29 | 2 | 2 | 4 | 1 |
| 30 | 0 | 2 | 2 | 1 |
| 31 | 0 | 2 | 2 | 1 |
| 32 | 2 | 2 | 4 | 1 |
| 33 | 0 | 2 | 3 | 1 |
| 34 | 2 | 2 | 4 | 1 |
| 35 | 0 | 2 | 3 | 1 |
| 36 | 2 | 2 | 0 | 1 |
| 37 | 0 | 2 | 3 | 1 |
| 38 | 2 | 2 | 4 | 1 |
| 39 | 2 | 2 | 4 | 1 |
| 40 | 0 | 2 | 1 | 0 |
| 41 | 0 | 2 | 2 | 1 |
| 42 | 2 | 2 | 4 | 1 |
| 43 | 2 | 2 | 3 | 1 |
| 44 | 0 | 2 | 3 | 0 |
| 45 | 0 | 2 | 2 | 0 |
| 46 | 2 | 2 | 4 | 1 |
| 47 | 1 | 2 | 4 | 0 |
| 48 | 0 | 2 | 2 | 1 |
| 49 | 0 | 2 | 1 | 0 |
| 50 | 1 | 2 | 4 | 0 |

TABLE 20d
Data for Serratia, particularly Serratia marcescens (continued)

| No. | AZT_pv_adj | CAX_pv_adj | CAZ_pv_adj | CFT_pv_adj | CP_pv_adj |
|---|---|---|---|---|---|
| 1 | 4.82E−04 | 5.24E−04 | 6.41E−03 | 7.29E−04 | 2.27E−05 |
| 2 | 1.58E−01 | 1.39E−01 | 2.26E−01 | 3.68E−02 | 6.01E−06 |
| 3 | 6.77E−04 | 8.30E−03 | 2.51E−02 | 1.65E−02 | 2.17E−05 |
| 4 | 8.92E−03 | 3.01E−03 | 3.16E−01 | 7.90E−03 | 2.95E−05 |
| 5 | 1.60E−02 | 4.77E−04 | 5.25E−03 | 1.36E−04 | 8.62E−06 |
| 6 | 1.22E−03 | 1.22E−03 | 1.24E−03 | 4.08E−04 | 8.62E−06 |
| 7 | 3.82E−02 | 3.55E−03 | 4.51E−02 | 2.17E−03 | 7.86E−05 |
| 8 | 1.48E−01 | 5.09E−03 | 6.93E−02 | 4.93E−03 | 1.10E−04 |
| 9 | 4.53E−02 | 5.02E−03 | 3.12E−01 | 2.64E−03 | 5.13E−04 |
| 10 | 6.43E−04 | 1.61E−04 | 7.67E−04 | 8.45E−05 | 1.56E−05 |
| 11 | 3.67E−05 | 1.56E−05 | 1.56E−05 | 5.02E−05 | 6.54E−03 |
| 12 | 1.44E−02 | 6.36E−03 | 4.83E−02 | 3.41E−03 | 2.36E−04 |
| 13 | 3.67E−05 | 1.56E−05 | 1.56E−05 | 5.02E−05 | 6.54E−03 |
| 14 | 4.07E−01 | 5.36E−02 | 7.62E−01 | 4.93E−02 | 5.86E−04 |
| 15 | 1.11E−03 | 7.67E−04 | 4.28E−02 | 1.04E−03 | 4.94E−05 |
| 16 | 6.43E−04 | 1.24E−03 | 2.38E−02 | 2.68E−03 | 1.82E−04 |
| 17 | 4.26E−03 | 1.84E−03 | 6.64E−02 | 4.93E−03 | 1.22E−04 |
| 18 | 1.73E−02 | 1.76E−04 | 2.04E−02 | 5.24E−04 | 6.30E−04 |
| 19 | 4.26E−03 | 1.84E−03 | 6.64E−02 | 4.93E−03 | 1.22E−04 |
| 20 | 9.20E−04 | 1.82E−04 | 2.34E−02 | 1.48E−04 | 1.69E−04 |
| 21 | 2.19E−02 | 6.70E−03 | 1 | 2.46E−02 | 7.82E−04 |
| 22 | 1.55E−03 | 4.30E−04 | 6.08E−02 | 5.90E−04 | 1.52E−04 |
| 23 | 4.85E−02 | 7.20E−04 | 1.01E−01 | 6.90E−04 | 3.71E−05 |
| 24 | 2.28E−03 | 2.41E−03 | 3.71E−05 | 4.23E−03 | 1.66E−01 |
| 25 | 1.01E−02 | 3.27E−04 | 5.44E−02 | 4.95E−04 | 7.62E−04 |
| 26 | 8.18E−04 | 3.20E−04 | 4.45E−02 | 3.27E−04 | 1.53E−04 |
| 27 | 2.82E−02 | 6.08E−03 | 2.88E−01 | 4.51E−02 | 1.10E−04 |
| 28 | 5.92E−02 | 3.15E−04 | 6.50E−01 | 1.11E−03 | 6.89E−03 |
| 29 | 1.66E−03 | 6.30E−04 | 8.28E−02 | 6.43E−04 | 3.33E−04 |
| 30 | 5.14E−02 | 1.60E−03 | 5.77E−02 | 2.64E−03 | 1.17E−03 |
| 31 | 5.14E−02 | 1.60E−03 | 5.77E−02 | 2.64E−03 | 1.17E−03 |
| 32 | 1.17E−03 | 3.15E−04 | 5.90E−02 | 3.27E−04 | 3.15E−04 |
| 33 | 1.07E−03 | 3.99E−03 | 2.05E−02 | 2.84E−03 | 5.02E−05 |
| 34 | 1.16E−03 | 5.89E−04 | 5.88E−02 | 6.07E−04 | 2.32E−04 |
| 35 | 4.11E−02 | 1.55E−03 | 5.76E−02 | 2.12E−03 | 1.15E−03 |
| 36 | 2.08E−02 | 2.04E−02 | 2.72E−01 | 3.09E−02 | 3.33E−04 |
| 37 | 5.14E−02 | 1.13E−03 | 5.80E−02 | 1.53E−03 | 1.50E−03 |
| 38 | 1.17E−03 | 3.15E−04 | 5.90E−02 | 3.27E−04 | 3.15E−04 |
| 39 | 4.30E−03 | 1.43E−03 | 1.74E−01 | 2.47E−03 | 9.38E−04 |
| 40 | 8.63E−02 | 1.24E−02 | 2.31E−01 | 1.12E−02 | 7.34E−04 |
| 41 | 5.14E−02 | 2.07E−03 | 5.80E−02 | 2.73E−03 | 1.50E−03 |
| 42 | 1.17E−03 | 3.15E−04 | 5.90E−02 | 3.27E−04 | 3.15E−04 |
| 43 | 7.04E−03 | 6.21E−03 | 3.82E−02 | 3.31E−03 | 1.04E−04 |
| 44 | 3.16E−01 | 1.58E−03 | 5.58E−01 | 1.57E−03 | 7.78E−04 |
| 45 | 2.46E−02 | 8.70E−03 | 2.31E−01 | 5.07E−03 | 1.80E−03 |
| 46 | 1.55E−03 | 7.82E−04 | 5.97E−02 | 8.10E−04 | 3.15E−04 |
| 47 | 2.96E−04 | 2.61E−03 | 5.49E−02 | 3.46E−03 | 7.18E−05 |
| 48 | 5.20E−02 | 2.61E−03 | 5.87E−02 | 3.46E−03 | 1.53E−03 |
| 49 | 2.15E−01 | 1.36E−02 | 2.60E−01 | 9.12E−03 | 1.92E−04 |
| 50 | 2.93E−03 | 7.18E−05 | 2.88E−02 | 7.18E−05 | 1.15E−03 |

TABLE 20e
Data for Serratia, particularly Serratia marcescens (continued)

| No. | GM_pv_adj | LVX_pv_adj | P/T_pv_adj | TO_pv_adj | T/S_pv_adj |
|---|---|---|---|---|---|
| 1 | 7.29E−04 | 3.70E−06 | 4.19E−03 | 5.65E−04 | 2.79E−04 |
| 2 | 3.92E−02 | 8.62E−06 | 5.32E−01 | 5.08E−02 | 9.68E−03 |
| 3 | 3.60E−02 | 6.01E−06 | 4.96E−02 | 9.86E−02 | 1.55E−03 |
| 4 | 2.38E−01 | 8.62E−06 | 4.51E−04 | 1.33E−01 | 5.10E−03 |
| 5 | 2.87E−01 | 4.05E−04 | 5.63E−05 | 3.66E−02 | 2.11E−02 |
| 6 | 9.01E−01 | 8.10E−04 | 6.41E−03 | 4.07E−02 | 1.63E−01 |
| 7 | 3.66E−02 | 1.33E−05 | 1.49E−02 | 1.15E−02 | 2.81E−02 |
| 8 | 1.16E−01 | 1.38E−05 | 1.31E−02 | 6.43E−02 | 5.52E−03 |
| 9 | 1.25E−02 | 1.56E−05 | 5.88E−02 | 1.37E−03 | 3.00E−02 |
| 10 | 2.58E−01 | 1.22E−04 | 9.05E−05 | 4.31E−02 | 3.97E−02 |
| 11 | 2.06E−01 | 3.60E−01 | 3.67E−05 | 1.87E−01 | 3.72E−01 |
| 12 | 5.33E−03 | 1.56E−05 | 6.87E−03 | 3.46E−03 | 8.20E−03 |
| 13 | 2.06E−01 | 3.60E−01 | 3.67E−05 | 1.87E−01 | 3.72E−01 |
| 14 | 4.95E−02 | 1.56E−05 | 1.34E−01 | 1.43E−01 | 2.07E−02 |
| 15 | 2.56E−03 | 2.17E−05 | 2.35E−03 | 3.96E−03 | 4.94E−04 |
| 16 | 3.89E−04 | 2.95E−05 | 1.52E−03 | 3.51E−03 | 2.43E−03 |
| 17 | 1.48E−01 | 3.14E−05 | 2.32E−04 | 8.78E−02 | 5.89E−04 |
| 18 | 3.24E−03 | 3.14E−05 | 2.41E−03 | 4.32E−03 | 4.63E−03 |
| 19 | 1.48E−01 | 3.14E−05 | 2.32E−04 | 8.78E−02 | 5.89E−04 |
| 20 | 2.78E−03 | 3.14E−05 | 1.10E−03 | 2.42E−03 | 6.75E−04 |
| 21 | 2.77E−02 | 3.44E−05 | 5.49E−02 | 1.12E−01 | 1.40E−03 |
| 22 | 5.63E−03 | 3.66E−05 | 3.16E−03 | 5.49E−03 | 3.24E−03 |
| 23 | 3.72E−03 | 4.07E−05 | 1.76E−03 | 4.82E−03 | 3.60E−03 |
| 24 | 2.22E−01 | 1 | 9.74E−03 | 1.18E−01 | 2.81E−01 |
| 25 | 4.82E−03 | 3.71E−05 | 7.82E−04 | 2.06E−02 | 1.08E−02 |
| 26 | 1.24E−03 | 3.78E−05 | 1.36E−03 | 2.34E−03 | 1.55E−03 |
| 27 | 5.36E−02 | 4.07E−05 | 4.63E−02 | 3.92E−02 | 1.38E−02 |
| 28 | 1.41E−01 | 7.82E−04 | 4.20E−05 | 3.04E−02 | 5.10E−03 |
| 29 | 2.77E−03 | 5.02E−05 | 1.37E−03 | 5.69E−03 | 2.21E−03 |
| 30 | 1.47E−02 | 5.02E−05 | 1.03E−02 | 3.27E−02 | 6.75E−03 |
| 31 | 1.47E−02 | 5.02E−05 | 1.03E−02 | 3.27E−02 | 6.75E−03 |
| 32 | 1.76E−03 | 5.02E−05 | 1.07E−03 | 4.09E−03 | 2.26E−03 |
| 33 | 2.27E−02 | 3.89E−04 | 4.16E−02 | 1.11E−02 | 6.71E−03 |
| 34 | 1.76E−03 | 5.02E−05 | 1.80E−03 | 3.17E−03 | 2.24E−03 |
| 35 | 1.48E−02 | 5.02E−05 | 8.05E−03 | 2.57E−02 | 6.70E−03 |
| 36 | 2.77E−03 | 5.02E−05 | 1.42E−02 | 2.41E−03 | 2.10E−04 |
| 37 | 1.48E−02 | 5.02E−05 | 5.52E−03 | 3.27E−02 | 6.89E−03 |
| 38 | 1.76E−03 | 5.02E−05 | 1.07E−03 | 4.09E−03 | 2.26E−03 |
| 39 | 1.31E−03 | 5.02E−05 | 5.93E−03 | 3.06E−03 | 2.21E−03 |
| 40 | 3.13E−02 | 5.02E−05 | 5.52E−03 | 1.02E−02 | 9.79E−02 |
| 41 | 1.48E−02 | 5.02E−05 | 1.06E−02 | 3.27E−02 | 6.89E−03 |
| 42 | 1.76E−03 | 5.02E−05 | 1.07E−03 | 4.09E−03 | 2.26E−03 |
| 43 | 9.28E−04 | 5.02E−05 | 6.47E−02 | 2.47E−03 | 1.76E−03 |
| 44 | 1.83E−02 | 5.88E−05 | 1.62E−03 | 1.34E−02 | 1.37E−02 |
| 45 | 9.13E−01 | 6.62E−05 | 6.12E−02 | 1.22E−01 | 1.54E−01 |
| 46 | 1.80E−03 | 7.16E−05 | 2.42E−03 | 4.20E−03 | 2.34E−03 |
| 47 | 4.20E−02 | 8.45E−05 | 9.22E−03 | 6.72E−03 | 1.09E−02 |
| 48 | 1.50E−02 | 7.18E−05 | 1.34E−02 | 4.14E−02 | 9.77E−03 |
| 49 | 4.30E−02 | 7.18E−05 | 3.73E−02 | 2.25E−02 | 1.57E−02 |
| 50 | 4.68E−03 | 1.51E−04 | 1.15E−03 | 2.57E−02 | 1.67E−02 |

TABLE 20f
Data for Serratia, particularly Serratia marcescens (continued)

| | | | | |
|---|---|---|---|---|
| | | Gene. | GenBank. | |
| No. | Locus.Tag | Symbol | Accession | Chromosome |

| | | | |
|---|---|---|---|
| 1 | Spro_0657 | | YP_001476891 |
| 2 | HMPREF0758_4528 | | ZP_06641192 |
| 3 | HMPREF0758_4282 | wrbA | ZP_06640946 |
| 4 | HMPREF0758_1761 | | ZP_06638425 |
| 5 | SSYM_0272 | | ZP_08040542 |
| 6 | SOD_m00050 | | ZP_06193534 |
| 7 | SOD_d02380 | | ZP_06191491 |
| 8 | Spro_3910 | | YP_001480133 |
| 9 | SOD_j00160 | | ZP_06193066 |
| 10 | SOD_m00140 | | ZP_06193543 |
| 11 | SSYM_0779 | | ZP_08038782 |
| 12 | Pat9b_2844 | | YP_004116700 |
| 13 | SSYM_0215 | | ZP_08040491 |
| 14 | Spro_1286 | | YP_001477518 |
| 15 | HMPREF0758_3087 | paaY | ZP_06639751 |
| 16 | SOD_b04490 | | ZP_06190513 |
| 17 | HMPREF0758_1765 | | ZP_06638429 |
| 18 | HMPREF0758_2630 | deoR | ZP_06639294 |
| 19 | HMPREF0758_1762 | vtaK | ZP_06638426 |
| 20 | HMPREF0758_4768 | yedI | ZP_06641432 |
| 21 | SOD_b01290 | | ZP_06190194 |
| 22 | HMPREF0758_3449 | mdtJ | ZP_06640113 |
| 23 | HMPREF0758_0340 | | ZP_06637004 |
| 24 | HMPREF0758_4122 | dnaC | ZP_06640786 |
| 25 | HMPREF0758_1641 | pepE | ZP_06638305 |
| 26 | SOD_a01780 | | ZP_06189226 |
| 27 | Spro_0998 | | YP_001477230 |
| 28 | HMPREF0758_1747 | | ZP_06638411 |
| 29 | SOD_b01600 | | ZP_06190225 |
| 30 | SOD_b01460 | | ZP_06190211 |
| 31 | SOD_c02470 | | ZP_06190898 |
| 32 | Spro_2856 | | YP_001479085 |
| 33 | Spro_1467 | | YP_001477699 |
| 34 | Spro_1011 | | YP_001477243 |
| 35 | SOD_d01640 | | ZP_06191418 |
| 36 | CSAG_04704 | | ZP_04558356 |
| 37 | Spro_0906 | | YP_001477140 |
| 38 | Spro_2853 | | YP_001479082 |
| 39 | Spro_4574 | | YP_001480795 |
| 40 | SOD_a08590 | | ZP_06189897 |
| 41 | SOD_d03490 | | ZP_06191602 |
| 42 | SOD_b05060 | | ZP_06190570 |
| 43 | HMPREF0758_1879 | yhcC | ZP_06638543 |
| 44 | HMPREF0758_0325 | apbE | ZP_06636989 |
| 45 | Spro_2867 | | YP_001479096 |
| 46 | Spro_0904 | | YP_001477138 |
| 47 | SOD_b01270 | | ZP_06190192 |
| 48 | Spro_4454 | | YP_001480676 |
| 49 | Spro_4262 | | YP_001480484 |
| 50 | HMPREF0758_2167 | hrpB | ZP_06638831 |

TABLE 20g
Data for Serratia, particularly Serratia marcescens (continued)

| No. | Start.Coord | End.Coord | Strand | Scaffold.ID |
|---|---|---|---|---|
| 1 | 721013 | 721786 | + | 640753091 |
| 2 | 113063 | 113683 | + | 647012891 |
| 3 | 196203 | 196802 | + | 647012889 |
| 4 | 14537 | 15286 | + | 647012864 |
| 5 | 1 | 622 | + | 651285392 |
| 6 | 5289 | 5756 | − | 647012829 |
| 7 | 276617 | 277270 | − | 647012820 |
| 8 | 4328564 | 4329223 | + | 640753091 |
| 9 | 10640 | 11029 | − | 647012826 |
| 10 | 12102 | 12680 | − | 647012829 |
| 11 | 54235 | 55095 | − | 651285075 |
| 12 | 3117915 | 3119003 | − | 649633106 |
| 13 | 1 | 203 | + | 651285334 |
| 14 | 1415512 | 1416153 | − | 640753091 |
| 15 | 166028 | 166624 | − | 647012881 |
| 16 | 563304 | 564224 | − | 647012818 |
| 17 | 17164 | 19063 | + | 647012864 |
| 18 | 1577 | 1921 | + | 647012878 |
| 19 | 15286 | 15990 | + | 647012864 |
| 20 | 357014 | 357931 | − | 647012891 |
| 21 | 207219 | 208373 | − | 647012818 |
| 22 | 144943 | 145311 | + | 647012883 |
| 23 | 46700 | 47314 | − | 647012846 |
| 24 | 40618 | 41358 | − | 647012889 |
| 25 | 66823 | 67548 | + | 647012859 |
| 26 | 183381 | 184292 | + | 647012817 |
| 27 | 1101888 | 1102271 | + | 640753091 |
| 28 | 1316 | 1822 | + | 647012864 |
| 29 | 243135 | 244313 | − | 647012818 |
| 30 | 227360 | 228310 | + | 647012818 |
| 31 | 276786 | 277940 | − | 647012819 |
| 32 | 3134756 | 3135724 | − | 640753091 |
| 33 | 1599118 | 1600077 | + | 640753091 |
| 34 | 1112389 | 1113570 | − | 640753091 |
| 35 | 196893 | 197789 | − | 647012820 |
| 36 | 17332 | 17544 | − | 646206740 |
| 37 | 1005118 | 1006293 | + | 640753091 |
| 38 | 3131862 | 3132830 | − | 640753091 |
| 39 | 5048349 | 5049131 | − | 640753091 |
| 40 | 927700 | 928224 | + | 647012817 |
| 41 | 395510 | 396043 | + | 647012820 |
| 42 | 616091 | 617041 | − | 647012818 |
| 43 | 107941 | 108870 | + | 647012865 |
| 44 | 30480 | 31511 | − | 647012846 |
| 45 | 3146974 | 3147780 | − | 640753091 |
| 46 | 1003277 | 1004080 | + | 640753091 |
| 47 | 202590 | 204044 | − | 647012818 |
| 48 | 4927194 | 4927727 | + | 640753091 |
| 49 | 4725710 | 4726606 | − | 640753091 |
| 50 | 70350 | 72779 | + | 647012869 |

### Example 11

### Shigella

The procedure was carried out as in Example 1, except that the following microorganisms were used:

Bacterial Strains

The inventors selected 442 Shigella strains, particularly Shigella boydii, Shigella dysenteriae, Shigella flexneri, Shigella sonnei and other Shigella species, from the microbiology strain collection at Siemens Healthcare Diagnostics (West Sacramento, Calif.) for susceptibility testing and whole genome sequencing.

From MetaRef, 730 centroids of Shigella species were used as reference sequences.

The results for Shigella species are shown in Tables 21 (corresponding to Table 1) and 22 (corresponding to Table 2).

TABLE 21a
Data for Shigella species

| | Scaffold.External. | | |
|---|---|---|---|
| No. | Accession | Scaffold.Name | best_pv |
| 1 | NZ_ADUB01000093 | Escherichia coli MS 175-1 E_coli175-1-1.0_Cont166.1: NZ_ADUB01000093 | 6.86676649351252e−56 |
| 2 | NZ_ADUD01000475 | Escherichia coli MS 196-1 E_coli196-1-1.0_Cont1343.1: NZ_ADUD01000475 | 4.14817893591733e−46 |
| 3 | N Z_ADWT01000006 | Escherichia coli MS 124-1 E_coliMS124-1-1.0.1_Cont5.1: NZ_ADWT01000006 | 4.14817893591733e−46 |
| 4 | AP011957 | Salmonella enterica subsp. enterica serovar Typhimurium str. T000240 | 1.24332752332433e−44 |
| | | DNA: AP011957 | |
| 5 | CP001383 | Shigella flexneri 2002017: CP001383 | 5.69762827081762e−37 |
| 6 | NZ_AAMJ01000001 | Shigella dysenteriae 1012, unfinished sequence: NZ_AAMJ01000001 | 3.04086824044829e−34 |
| 7 | NZ_ADUB01000227 | Escherichia coli MS 175-1 E_coli175-1-1.0_Cont422.2: NZ_ADUB01000227 | 3.04086824044829e−34 |
| 8 | NZ_AAMJ01000001 | Shigella dysenteriae 1012, unfinished sequence: NZ_AAMJ01000001 | 5.67507153238613e−34 |
| 9 | CP001383 | Shigella flexneri 2002017: CP001383 | 1.46728061376833e−26 |
| 10 | NZ_ADUB01000365 | Escherichia coli MS 175-1 E_coli175-1-1.0_Cont823.2: NZ_ADUB01000365 | 4.97695865217724e−26 |
| 11 | NZ_ADUB01000054 | Escherichia coli MS 175-1 E_coli175-1-1.0_Cont100.1: NZ_ADUB01000054 | 1.31351033204593e−24 |
| 12 | CP001383 | Shigella flexneri 2002017: CP001383 | 1.66700615646725e−24 |
| 13 | NC_013509 | Edwardsiella tarda EIB202 plasmid pEIB202: NC_013509 | 3.081692811393e−24 |
| 14 | NZ_ABKX01000001 | Escherichia albertii TW07627, unfinished sequence: NZ_ABKX01000001 | 3.45432516278107e−24 |
| 15 | NC_007384 | Shigella sonnei Ss046: NC_007384 | 4.84345172785563e−23 |
| 16 | CP001383 | Shigella flexneri 2002017: CP001383 | 1.59570448713613e−17 |
| 17 | NZ_ADUT01000002 | Shigella dysenteriae 1617 gss1617.assembly.11: NZ_ADUT01000002 | 4.66906657298136e−17 |
| 18 | NZ_ABKX01000001 | Escherichia albertii TW07627, unfinished sequence: NZ_ABKX01000001 | 5.88113462200155e−17 |
| 19 | NZ_ADTR01000390 | Escherichia coli MS 21-1 E_coli21-1-1.0_Cont744.1: NZ_ADTR01000390 | 7.05214682414966e−16 |
| 20 | NZ_ADUT01000002 | Shigella dysenteriae 1617 gss1617.assembly.11: NZ_ADUT01000002 | 3.49647006284355e−15 |
| 21 | NC_006856 | Salmonella enterica subsp. enterica serovar Choleraesuis str. SC-B67 | 3.88428414470937e−13 |
| | | plasmid pSC138: NC_006856 | |
| 22 | NC_011743 | Escherichia fergusonii ATCC 35469 plasmid pEFER: NC_011743 | 6.18164438611072e−13 |
| 23 | NZ_ADUT01000048 | Shigella dysenteriae 1617 gss1617.assembly.53: NZ_ADUT01000048 | 6.40162543140323e−12 |
| 24 | NZ_ADUT01000002 | Shigella dysenteriae 1617 gss1617.assembly.11: NZ_ADUT01000002 | 1.53869362793954e−11 |
| 25 | NC_011745 | Escherichia coli ED1a: NC_011745 | 2.16302499579491e−11 |
| 26 | NZ_ADUT01000002 | Shigella dysenteriae 1617 gss1617.assembly.11: NZ_ADUT01000002 | 2.30761086733495e−11 |
| 27 | NZ_ABKY02000005 | Escherichia coli O157:H7 str. TW14588, unfinished sequence: | 4.43701528503845e−11 |
| | | NZ_ABKY02000005 | |
| 28 | NZ_ADUT01000002 | Shigella dysenteriae 1617 gss1617.assembly.11: NZ_ADUT01000002 | 8.04473895167901e−11 |
| 29 | CP002729 | Escherichia coli UMNK88: CP002729 | 1.42165494676851e−10 |
| 30 | NC_011742 | Escherichia coli S88: NC_011742 | 2.05879614239143e−10 |
| 31 | NZ_ABAK01000021 | Salmonella enterica subsp. enterica serovar Kentucky str. CVM29188, | 2.13757750700881e−10 |
| | | unfinished sequence: NZ_ABAK01000021 | |
| 32 | NC_013509 | Edwardsiella tarda EIB202 plasmid pEIB202: NC_013509 | 4.04449595701974e−10 |
| 33 | NZ_ABHS01000025 | Escherichia coli O157:H7 str. EC4486, unfinished sequence: | 4.85503165975477e−10 |
| | | NZ_ABHS01000025 | |
| 34 | NC_012759 | Escherichia coli BW2952: NC_012759 | 4.85503165975477e−10 |
| 35 | NZ_ADUD01000718 | Escherichia coli MS 196-1 E_coli196-1-1.0_Cont2124.2: NZ_ADUD01000718 | 5.8673304681529e−10 |
| 36 | NC_002655 | Escherichia coli O157:H7 EDL933: NC_002655 | 7.67538561798816e−10 |
| 37 | CP001509 | Escherichia coli BL21(DE3): CP001509 | 8.69490265401296e−10 |
| 38 | FP929040 | Enterobacter cloacae subsp. cloacae NCTC 9394 draft genome.: FP929040 | 1.59741374269166e−09 |
| 39 | NZ_AFBO01000392 | Klebsiella sp.MS 92-3 K_spMS92-3-1.0_Cont773.2: NZ_AFBO01000392 | 1.59741374269166e−09 |
| 40 | NZ_ADTQ01000190 | Escherichia coli MS 187-1 E_coli187-1-1.0_Cont287.1: NZ_ADTQ01000190 | 1.59741374269166e−09 |
| 41 | NZ_ACZD01000015 | Klebsiella pneumoniae subsp. rhinoscleromatis ATCC 13884 contig00015: | 1.59741374269166e−09 |
| | | NZ_ACZD01000015 | |
| 42 | NC_007384 | Shigella sonnei Ss046: NC_007384 | 1.59741374269166e−09 |
| 43 | NZ_ADUT01000040 | Shigella dysenteriae 1617 gss1617.assembly.46: NZ_ADUT01000040 | 1.75588873952222e−09 |
| 44 | NZ_CAAZ01000109 | Salmonella enterica subsp. enterica serovar Typhi str. E98-3139, | 4.20666708065652e−09 |
| | | unfinished sequence: NZ_CAAZ01000109 | |
| 45 | NZ_AAMJ01000012 | Shigella dysenteriae 1012, unfinished sequence: NZ_AAMJ01000012 | 5.072437403203e−09 |
| 46 | NC_010658 | Shigella boydii CDC 3083-94: NC_010658 | 8.52438957707961e−09 |
| 47 | FN554766 | Escherichia coli 042: FN554766 | 8.7610944464379e−09 |
| 48 | NZ_ADTL01000219 | Escherichia coli MS 115-1 E_coli115-1-1.0_Cont569.1: NZ_ADTL01000219 | 8.7610944464379e−09 |
| 49 | NZ_ABJT01000012 | Escherichia coli O157:H7 str. EC4024, unfinished sequence: | 9.3358254121691e−09 |
| | | NZ_ABJT01000012 | |
| 50 | NZ_AAJV01000016 | Escherichia coli E22, unfinished sequence: NZ_AAJV01000016 | 1.17232056049228e−08 |

TABLE 21b
Data for Shigella species (continued)

| No. | sign_phenos | sign_phenos_class | best_pheno | best_pheno_class |
|---|---|---|---|---|
| 1 | AM; A/S; AUG; T/S | lactam; other | AUG | lactam |
| 2 | AM; A/S; AUG; T/S | lactam; other | AUG | lactam |
| 3 | AM; A/S; AUG; T/S | lactam; other | AUG | lactam |
| 4 | AM; A/S; AUG; T/S | lactam; other | AUG | lactam |
| 5 | AM; A/S; AUG | lactam | AUG | lactam |
| 6 | T/S | other | T/S | other |
| 7 | T/S | other | T/S | other |
| 8 | T/S | other | T/S | other |
| 9 | T/S | other | T/S | other |
| 10 | T/S | other | T/S | other |
| 11 | AM; A/S; AUG; T/S | lactam; other | AUG | lactam |
| 12 | AM; A/S; AUG; T/S | lactam; other | AUG | lactam |
| 13 | A/S; AUG; T/S | lactam; other | T/S | other |
| 14 | AM; A/S; AUG; T/S | lactam; other | AUG | lactam |
| 15 | AM; A/S; AUG; T/S | lactam; other | AUG | lactam |
| 16 | T/S | other | T/S | other |
| 17 | AM; A/S | lactam | A/S | lactam |
| 18 | AM; A/S; AUG | lactam | AUG | lactam |
| 19 | AM; A/S | lactam | A/S | lactam |
| 20 | AM; A/S | lactam | A/S | lactam |
| 21 | AM; A/S | lactam | A/S | lactam |
| 22 | T/S | other | T/S | other |
| 23 | AM; A/S; AUG | lactam | AUG | lactam |
| 24 | AM; A/S | lactam | A/S | lactam |
| 25 | AM; A/S; AUG; T/S | lactam; other | AM | lactam |
| 26 | AM; A/S | lactam | A/S | lactam |
| 27 | AM; A/S; AUG | lactam | A/S | lactam |
| 28 | AM; A/S | lactam | A/S | lactam |
| 29 | AUG | lactam | AUG | lactam |
| 30 | T/S | other | T/S | other |
| 31 | AM; A/S | lactam | A/S | lactam |
| 32 | T/S | other | T/S | other |
| 33 | AM; A/S | lactam | A/S | lactam |
| 34 | AM; A/S | lactam | AM | lactam |
| 35 | AM; A/S | lactam | A/S | lactam |
| 36 | AUG | lactam | AUG | lactam |
| 37 | AM; A/S | lactam | AM | lactam |
| 38 | AM; A/S | lactam | AM | lactam |
| 39 | AM; A/S | lactam | AM | lactam |
| 40 | AM; A/S | lactam | AM | lactam |
| 41 | AM; A/S | lactam | AM | lactam |
| 42 | AM; A/S | lactam | AM | lactam |
| 43 | AUG | lactam | AUG | lactam |
| 44 | AUG | lactam | AUG | lactam |
| 45 | AUG | lactam | AUG | lactam |
| 46 | AUG | lactam | AUG | lactam |
| 47 | AUG | lactam | AUG | lactam |
| 48 | AUG | lactam | AUG | lactam |
| 49 | AM; A/S | lactam | AM | lactam |
| 50 | AM; A/S; T/S | lactam; other | AM | lactam |

TABLE 21c
Data for Shigella species (continued)

| No. | num_lactam | num_other | AM_pv_adj | A/S_pv_adj | AUG_pv_adj | T/S_pv_adj |
|---|---|---|---|---|---|---|
| 1 | 3 | 1 | 2.50E−24 | 2.78E−27 | 6.87E−56 | 6.43E−03 |
| 2 | 3 | 1 | 3.96E−18 | 5.18E−21 | 4.15E−46 | 3.89E−03 |
| 3 | 3 | 1 | 1.61E−18 | 1.80E−21 | 4.15E−46 | 2.75E−03 |
| 4 | 3 | 1 | 1.48E−18 | 1.80E−21 | 1.24E−44 | 5.45E−03 |
| 5 | 3 | 0 | 6.38E−20 | 1.76E−12 | 5.70E−37 | 1.36E−02 |
| 6 | 0 | 1 | 4.19E−01 | 7.59E−01 | 1.18E−01 | 3.04E−34 |
| 7 | 0 | 1 | 4.19E−01 | 7.59E−01 | 1.18E−01 | 3.04E−34 |
| 8 | 0 | 1 | 4.14E−01 | 6.88E−01 | 1.42E−01 | 5.68E−34 |
| 9 | 0 | 1 | 5.86E−01 | 9.62E−01 | 1.89E−01 | 1.47E−26 |
| 10 | 0 | 1 | 6.64E−01 | 8.98E−01 | 3.47E−01 | 4.98E−26 |
| 11 | 3 | 1 | 1.30E−12 | 6.35E−09 | 1.31E−24 | 1.10E−04 |
| 12 | 3 | 1 | 8.43E−13 | 4.07E−09 | 1.67E−24 | 3.04E−05 |
| 13 | 2 | 1 | 5.59E−02 | 5.39E−03 | 8.83E−03 | 3.08E−24 |
| 14 | 3 | 1 | 6.77E−13 | 3.67E−09 | 3.45E−24 | 4.57E−05 |
| 15 | 3 | 1 | 1.11E−11 | 1.11E−08 | 4.84E−23 | 3.29E−12 |
| 16 | 0 | 1 | 7.13E−01 | 9.56E−01 | 8.48E−02 | 1.60E−17 |
| 17 | 2 | 0 | 1.56E−14 | 4.67E−17 | 6.49E−01 | 1.06E−01 |
| 18 | 3 | 0 | 7.73E−05 | 2.34E−06 | 5.88E−17 | 9.61E−01 |
| 19 | 2 | 0 | 5.63E−14 | 7.05E−16 | 9.63E−01 | 3.40E−02 |
| 20 | 2 | 0 | 4.77E−14 | 3.50E−15 | 1 | 7.62E−02 |
| 21 | 2 | 0 | 3.45E−11 | 3.88E−13 | 6.47E−01 | 3.82E−02 |
| 22 | 0 | 1 | 5.80E−01 | 2.16E−01 | 2.14E−01 | 6.18E−13 |
| 23 | 3 | 0 | 5.45E−03 | 1.12E−03 | 6.40E−12 | 1.67E−01 |
| 24 | 2 | 0 | 2.67E−09 | 1.54E−11 | 4.21E−01 | 7.26E−02 |
| 25 | 3 | 1 | 2.16E−11 | 8.03E−10 | 4.80E−05 | 1.95E−03 |
| 26 | 2 | 0 | 3.72E−09 | 2.31E−11 | 5.37E−01 | 1.18E−01 |
| 27 | 3 | 0 | 2.93E−08 | 4.44E−11 | 1.86E−03 | 1.59E−01 |
| 28 | 2 | 0 | 7.34E−09 | 8.04E−11 | 6.55E−01 | 7.07E−02 |
| 29 | 1 | 0 | 2.46E−01 | 5.71E−02 | 1.42E−10 | 5.27E−01 |
| 30 | 0 | 1 | 1.01E−01 | 3.04E−02 | 5.99E−01 | 2.06E−10 |
| 31 | 2 | 0 | 7.76E−09 | 2.14E−10 | 3.71E−01 | 1.76E−01 |
| 32 | 0 | 1 | 6.37E−01 | 2.87E−01 | 2.49E−01 | 4.04E−10 |
| 33 | 2 | 0 | 5.22E−08 | 4.86E−10 | 2.71E−01 | 2.09E−01 |
| 34 | 2 | 0 | 4.86E−10 | 3.14E−08 | 5.20E−02 | 5.27E−01 |
| 35 | 2 | 0 | 1.90E−08 | 5.87E−10 | 4.20E−01 | 1.73E−01 |
| 36 | 1 | 0 | 1.02E−01 | 2.02E−02 | 7.68E−10 | 3.60E−01 |
| 37 | 2 | 0 | 8.69E−10 | 5.22E−08 | 6.38E−02 | 5.96E−01 |
| 38 | 2 | 0 | 1.60E−09 | 8.74E−08 | 6.39E−02 | 5.96E−01 |
| 39 | 2 | 0 | 1.60E−09 | 8.74E−08 | 6.39E−02 | 5.96E−01 |
| 40 | 2 | 0 | 1.60E−09 | 8.74E−08 | 6.39E−02 | 5.96E−01 |
| 41 | 2 | 0 | 1.60E−09 | 8.74E−08 | 6.39E−02 | 5.96E−01 |
| 42 | 2 | 0 | 1.60E−09 | 8.74E−08 | 6.39E−02 | 5.96E−01 |
| 43 | 1 | 0 | 4.98E−01 | 1.34E−01 | 1.76E−09 | 3.44E−01 |
| 44 | 1 | 0 | 5.00E−01 | 1.36E−01 | 4.21E−09 | 4.06E−01 |
| 45 | 1 | 0 | 3.20E−01 | 7.93E−02 | 5.07E−09 | 6.63E−01 |
| 46 | 1 | 0 | 1 | 5.81E−01 | 8.52E−09 | 4.31E−01 |
| 47 | 1 | 0 | 2.42E−01 | 8.26E−02 | 8.76E−09 | 5.20E−01 |
| 48 | 1 | 0 | 2.42E−01 | 8.26E−02 | 8.76E−09 | 5.20E−01 |
| 49 | 2 | 0 | 9.34E−09 | 1.80E−06 | 2.16E−01 | 7.92E−02 |
| 50 | 2 | 1 | 1.17E−08 | 4.60E−05 | 3.74E−02 | 7.92E−04 |

TABLE 21d
Data for Shigella species (continued)

| No. | Locus.Tag | Gene.Symbol | GenBank.Accession | Chromosome |
|---|---|---|---|---|
| 1 | HMPREF9547_01191 | | ZP_07167682 | |
| 2 | HMPREF9551_04221 | | ZP_07191576 | |
| 3 | HMPREF9347_01086 | | ZP_07208640 | |
| 4 | STMDT12_C39170 | | BAJ38860 | |
| 5 | SFxv_1168 | | ADA73425 | |
| 6 | Sdys1_01000012 | | ZP_00922750 | |
| 7 | HMPREF9547_02735 | | ZP_07169192 | |
| 8 | Sdys1_01000013 | | ZP_00922751 | |
| 9 | SFxv_4146 | | ADA76106 | |
| 10 | HMPREF9547_04432 | | ZP_07170853 | |
| 11 | HMPREF9547_00721 | | ZP_07167228 | |
| 12 | SFxv_1141 | | ADA73399 | |
| 13 | ETAE_p041 | folP | YP_003297635 | plasmid pEIB202 |
| 14 | ESCAB7627_0644 | | ZP_02900823 | |
| 15 | SSON_3896 | aadA | YP_312670 | |
| 16 | SFxv_4148 | | ADA76108 | |
| 17 | SD1617_0118 | | ZP_07678322 | |
| 18 | ESCAB7627_0650 | | ZP_02900555 | |
| 19 | HMPREF9530_04295 | | ZP_07154150 | |
| 20 | SD1617_0032 | | ZP_07678236 | |
| 21 | SC109 | ccgA2 | YP_209411 | plasmid pSC138 |
| 22 | pEFER_0049 | | YP_002394596 | plasmid pEFER |
| 23 | SD1617_4863 | | ZP_07682961 | |
| 24 | SD1617_0123 | | ZP_07678327 | |
| 25 | ECED1_1050 | | YP_002397074 | |
| 26 | SD1617_0043 | | ZP_07678247 | |
| 27 | ESCCO14588_3315 | | ZP_03445386 | |
| 28 | SD1617_0122 | | ZP_07678326 | |
| 29 | UMNK88_1471 | | AEE56075 | |
| 30 | ECS88_2899 | rusA | YP_002392541 | |
| 31 | Sente_010100023234 | | ZP_02563153 | |
| 32 | ETAE_p040 | | YP_003297634 | plasmid pEIB202 |
| 33 | EcolO157_010100025333 | | ZP_02795604 | |
| 34 | BWG_3989 | fecR | YP_002929180 | |
| 35 | HMPREF9551_05588 | | ZP_07192915 | |
| 36 | Z1242 | dmsC | NP_286773 | |
| 37 | ECD_04153 | fecE | ACT45937 | |
| 38 | ENC_36610 | | CBK86965 | |
| 39 | HMPREF9538_03067 | | ZP_08305382 | |
| 40 | HMPREF9550_02956 | | ZP_07146070 | |
| 41 | HMPREF0484_0234 | fecA | ZP_06013219 | |
| 42 | SSON_4460 | fecD | YP_313172 | |
| 43 | SD1617_4069 | | ZP_07682183 | |
| 44 | Salmonellentericaenterica_010100012935 | | ZP_03412076 | |
| 45 | Sdys1_01002088 | | ZP_00921058 | |
| 46 | SbBS512_E0266 | | YP_001879059 | |
| 47 | EC042_2952 | | CBG35781 | |
| 48 | HMPREF9540_02755 | | ZP_07135552 | |
| 49 | EscherichcoliO157_010100012850 | | ZP_03082702 | |
| 50 | EcolE2_01002516 | | ZP_00729149 | |

TABLE 21e
Data for Shigella species (continued)

| No. | Start.Coord | End.Coord | Strand | Scaffold.ID |
|---|---|---|---|---|
| 1 | 6987 | 7817 | + | 648291751 |
| 2 | 10102 | 10662 | − | 648292991 |
| 3 | 2726 | 5692 | − | 648294279 |
| 4 | 4067283 | 4067942 | + | 651053007 |
| 5 | 1128245 | 1129249 | + | 646862363 |
| 6 | 12375 | 12701 | − | 638365528 |
| 7 | 4692 | 5189 | − | 648291885 |
| 8 | 14252 | 14776 | + | 638365528 |
| 9 | 3950954 | 3952570 | − | 646862363 |
| 10 | 8898 | 9719 | − | 648292023 |
| 11 | 5423 | 6016 | − | 648291712 |
| 12 | 1107342 | 1107968 | + | 646862363 |
| 13 | 32101 | 32916 | − | 646311959 |
| 14 | 233492 | 234697 | − | 641784575 |
| 15 | 4110458 | 4111246 | − | 640427102 |
| 16 | 3954100 | 3955767 | − | 646862363 |
| 17 | 94539 | 95570 | + | 649994652 |
| 18 | 238788 | 239996 | − | 641784575 |
| 19 | 2693 | 3169 | + | 648290614 |
| 20 | 9270 | 9803 | − | 649994652 |
| 21 | 84616 | 85080 | − | 637000354 |
| 22 | 48658 | 49503 | + | 643692014 |
| 23 | 80678 | 81835 | − | 649994698 |
| 24 | 98900 | 99016 | − | 649994652 |
| 25 | 1066134 | 1066832 | + | 643348624 |
| 26 | 17832 | 18008 | + | 649994652 |
| 27 | 383590 | 384330 | − | 642987534 |
| 28 | 98413 | 98754 | − | 649994652 |
| 29 | 1420478 | 1421209 | − | 651053088 |
| 30 | 2863316 | 2863705 | − | 644736332 |
| 31 | 17544 | 17792 | + | 641746180 |
| 32 | 31237 | 32040 | − | 646311959 |
| 33 | 65402 | 65668 | − | 641781914 |
| 34 | 4453522 | 4454475 | − | 644736346 |
| 35 | 1327 | 1764 | − | 648293234 |
| 36 | 1164368 | 1165231 | + | 637000062 |
| 37 | 4424087 | 4424854 | − | 646862369 |
| 38 | 3698592 | 3699494 | − | 650378029 |
| 39 | 2039 | 3037 | − | 651333309 |
| 40 | 271 | 792 | + | 648290104 |
| 41 | 4245 | 6485 | + | 647009762 |
| 42 | 4731572 | 4732528 | − | 640427102 |
| 43 | 500702 | 501520 | + | 649994690 |
| 44 | 43535 | 45085 | + | 643042589 |
| 45 | 19505 | 20209 | − | 638365539 |
| 46 | 266116 | 266985 | − | 641522725 |
| 47 | 3140492 | 3141238 | − | 646862421 |
| 48 | 1421 | 1912 | − | 648288078 |
| 49 | 103370 | 103972 | + | 642792886 |
| 50 | 35676 | 36281 | − | 638358744 |

TABLE 22a
Data for Shigella species

| No. | Scaffold.External.Accession | Scaffold.Name | best_pv |
|---|---|---|---|
| 1 | NZ_ADUB01000093 | Escherichia coli MS 175-1 E_coli175-1-1.0_Cont166.1: NZ_ADUB01000093 | 6.86676649351252e−56 |
| 2 | NZ_ADUD01000475 | Escherichia coli MS 196-1 E_coli196-1-1.0_Cont1343.1: NZ_ADUD01000475 | 4.14817893591733e−46 |
| 3 | NZ_ADWT01000006 | Escherichia coli MS 124-1 E_coliMS124-1-1.0.1_Cont5.1: NZ_ADWT01000006 | 4.14817893591733e−46 |
| 4 | AP011957 | Salmonella enterica subsp. enterica serovar Typhimurium str. T000240 | 1.24332752332433e−44 |
| | | DNA: AP011957 | |
| 5 | CP001383 | Shigella flexneri 2002017: CP001383 | 5.69762827081762e−37 |
| 6 | NZ_AAMJ01000001 | Shigella dysenteriae 1012, unfinished sequence: NZ_AAMJ01000001 | 3.04086824044829e−34 |
| 7 | NZ_ADUB01000227 | Escherichia coli MS 175-1 E_coli175-1-1.0_Cont422.2: NZ_ADUB01000227 | 3.04086824044829e−34 |
| 8 | NZ_AAMJ01000001 | Shigella dysenteriae 1012, unfinished sequence: NZ_AAMJ01000001 | 5.67507153238613e−34 |
| 9 | CP001383 | Shigella flexneri 2002017: CP001383 | 1.46728061376833e−26 |
| 10 | NZ_ADUB01000365 | Escherichia coli MS 175-1 E_coli175-1-1.0_Cont823.2: NZ_ADUB01000365 | 4.97695865217724e−26 |
| 11 | NZ_ADUB01000054 | Escherichia coli MS 175-1 E_coli175-1-1.0_Cont100.1: NZ_ADUB01000054 | 1.31351033204593e−24 |
| 12 | CP001383 | Shigella flexneri 2002017: CP001383 | 1.66700615646725e−24 |
| 13 | NC_013509 | Edwardsiella tarda EIB202 plasmid pEIB202: NC_013509 | 3.081692811393e−24 |
| 14 | NZ_ABKX01000001 | Escherichia albertii TW07627, unfinished sequence: NZ_ABKX01000001 | 3.45432516278107e−24 |
| 15 | NC_007384 | Shigella sonnei Ss046: NC_007384 | 4.84345172785563e−23 |
| 16 | CP001383 | Shigella flexneri 2002017: CP001383 | 1.59570448713613e−17 |
| 17 | NZ_ABKX01000001 | Escherichia albertii TW07627, unfinished sequence: NZ_ABKX01000001 | 5.88113462200155e−17 |
| 18 | NC_011743 | Escherichia fergusonii ATCC 35469 plasmid pEFER: NC_011743 | 6.18164438611072e−13 |
| 19 | NZ_ADUT01000048 | Shigella dysenteriae 1617 gss1617.assembly.53: NZ_ADUT01000048 | 6.40162543140323e−12 |
| 20 | NC_011745 | Escherichia coli ED1a: NC_011745 | 2.16302499579491e−11 |
| 21 | NZ_ABKY02000005 | Escherichia coli O157:H7 str. TW14588, unfinished sequence: | 4.43701528503845e−11 |
| | | NZ_ABKY02000005 | |
| 22 | NC_011742 | Escherichia coli S88: NC_011742 | 2.05879614239143e−10 |
| 23 | NC_013509 | Edwardsiella tarda EIB202 plasmid pEIB202: NC_013509 | 4.04449595701974e−10 |
| 24 | NZ_AAJV01000016 | Escherichia coli E22, unfinished sequence: NZ_AAJV01000016 | 1.17232056049228e−08 |
| 25 | NC_012731 | Klebsiella pneumoniae NTUH-K2044: NC_012731 | 1.74593394018129e−08 |
| 26 | NC_011740 | Escherichia fergusonii ATCC 35469: NC_011740 | 2.08793524887857e−08 |
| 27 | NC_011740 | Escherichia fergusonii ATCC 35469: NC_011740 | 3.5987076197447e−08 |
| 28 | NZ_GG749327 | Escherichia coli B354 genomic scaffold supercont1.2: NZ_GG749327 | 3.5987076197447e−08 |
| 29 | NZ_ADTP01000357 | Escherichia coli MS 69-1 E_coli69-1-1.0_Cont727.1: NZ_ADTP01000357 | 3.5987076197447e−08 |
| 30 | NC_008258 | Shigella flexneri 5 str. 8401: NC_008258 | 3.5987076197447e−08 |
| 31 | NC_008258 | Shigella flexneri 5 str. 8401: NC_008258 | 3.6014669209108e−08 |
| 32 | NC_011093 | Salmonella enterica subsp. enterica serovar Schwarzengrund str. | 4.21633659816666e−08 |
| | | CVM19633 plasmid pCVM19633_4: NC_011093 | |
| 33 | NZ_ABWL01000002 | Citrobacter youngae ATCC 29220 C_sp-1.0_Cont0.2: NZ_ABWL01000002 | 5.87968902033994e−08 |
| 34 | NC_011740 | Escherichia fergusonii ATCC 35469: NC_011740 | 5.87968902033994e−08 |
| 35 | NZ_ADUT01000013 | Shigella dysenteriae 1617 gss1617.assembly.21: NZ_ADUT01000013 | 5.87968902033994e−08 |
| 36 | NC_010658 | Shigella boydii CDC 3083-94: NC_010658 | 5.87968902033994e−08 |
| 37 | NC_011740 | Escherichia fergusonii ATCC 35469: NC_011740 | 5.87968902033994e−08 |
| 38 | NZ_ABEJ01000032 | Salmonella enterica subsp. enterica serovar Schwarzengrund str. SL480, | 6.12813910192695e−08 |
| | | unfinished sequence: NZ_ABEJ01000032 | |
| 39 | NC_009345 | Shigella sonnei Ss046 plasmid pSS046_spA: NC_009345 | 7.76943438061442e−08 |
| 40 | NC_011740 | Escherichia fergusonii ATCC 35469: NC_011740 | 8.81464918585737e−08 |
| 41 | NZ_ABKX01000001 | Escherichia albertii TW07627, unfinished sequence: NZ_ABKX01000001 | 1.19080570281449e−07 |
| 42 | CP001671 | Escherichia coli ABU 83972: CP001671 | 1.50273530777206e−07 |
| 43 | CP001637 | Escherichia coli DH1: CP001637 | 2.0405961424353e−07 |
| 44 | NC_004741 | Shigella flexneri 2a str. 2457T: NC_004741 | 2.0405961424353e−07 |
| 45 | NZ_AAKB01000013 | Escherichia coli 53638, unfinished sequence: NZ_AAKB01000013 | 2.08917442901333e−07 |
| 46 | NZ_ADUL01000041 | Escherichia coli 2362-75 gec2362.assembly.47: NZ_ADUL01000041 | 2.54562372489639e−07 |
| 47 | NZ_ABKX01000001 | Escherichia albertii TW07627, unfinished sequence: NZ_ABKX01000001 | 3.82958267583033e−07 |
| 48 | NZ_ADTR01000289 | Escherichia coli MS 21-1 E_coli21-1-1.0_Cont515.2: NZ_ADTR01000289 | 3.97520776634592e−07 |
| 49 | NC_010658 | Shigella boydii CDC 3083-94: NC_010658 | 4.00202730594618e−07 |
| 50 | NZ_GG657366 | Citrobacter sp. 30_2 genomic scaffold supercont1.1: NZ_GG657366 | 4.18196454987758e−07 |

TABLE 22b
Data for Shigella species

| No. | sign_phenos | sign_phenos_class | best_pheno | best_pheno_class |
|---|---|---|---|---|
| 1 | AM; A/S; AUG; T/S | lactam; other | AUG | lactam |
| 2 | AM; A/S; AUG; T/S | lactam; other | AUG | lactam |
| 3 | AM; A/S; AUG; T/S | lactam; other | AUG | lactam |
| 4 | AM; A/S; AUG; T/S | lactam; other | AUG | lactam |
| 5 | AM; A/S; AUG | lactam | AUG | lactam |
| 6 | T/S | other | T/S | other |
| 7 | T/S | other | T/S | other |
| 8 | T/S | other | T/S | other |
| 9 | T/S | other | T/S | other |
| 10 | T/S | other | T/S | other |
| 11 | AM; A/S; AUG; T/S | lactam; other | AUG | lactam |
| 12 | AM; A/S; AUG; T/S | lactam; other | AUG | lactam |
| 13 | A/S; AUG; T/S | lactam; other | T/S | other |
| 14 | AM; A/S; AUG; T/S | lactam; other | AUG | lactam |
| 15 | AM; A/S; AUG; T/S | lactam; other | AUG | lactam |
| 16 | T/S | other | T/S | other |
| 17 | AM; A/S; AUG | lactam | AUG | lactam |
| 18 | T/S | other | T/S | other |
| 19 | AM; A/S; AUG | lactam | AUG | lactam |
| 20 | AM; A/S; AUG; T/S | lactam; other | AM | lactam |
| 21 | AM; A/S; AUG | lactam | A/S | lactam |
| 22 | T/S | other | T/S | other |
| 23 | T/S | other | T/S | other |
| 24 | AM; A/S; T/S | lactam; other | AM | lactam |
| 25 | AM; A/S; T/S | lactam; other | AM | lactam |
| 26 | AM; A/S; AUG | lactam | AM | lactam |
| 27 | AM; A/S; T/S | lactam; other | AM | lactam |
| 28 | AM; A/S; T/S | lactam; other | AM | lactam |
| 29 | AM; A/S; T/S | lactam; other | AM | lactam |
| 30 | AM; A/S; T/S | lactam; other | AM | lactam |
| 31 | AM; A/S; AUG | lactam | AUG | lactam |
| 32 | AM; A/S; AUG | lactam | AM | lactam |
| 33 | AM; A/S; T/S | lactam; other | A/S | lactam |
| 34 | AM; A/S; T/S | lactam; other | A/S | lactam |
| 35 | AM; A/S; T/S | lactam; other | A/S | lactam |
| 36 | AM; A/S; T/S | lactam; other | A/S | lactam |
| 37 | AM; A/S; T/S | lactam; other | A/S | lactam |
| 38 | AM; A/S; AUG; T/S | lactam; other | T/S | other |
| 39 | AM; A/S; AUG; T/S | lactam; other | T/S | other |
| 40 | AM; A/S; AUG; T/S | lactam; other | AUG | lactam |
| 41 | AM; A/S; AUG | lactam | A/S | lactam |
| 42 | AM; A/S; T/S | lactam; other | AM | lactam |
| 43 | AM; A/S; AUG | lactam | A/S | lactam |
| 44 | AM; A/S; AUG | lactam | A/S | lactam |
| 45 | AM; A/S; AUG | lactam | AM | lactam |
| 46 | AM; A/S; AUG; T/S | lactam; other | A/S | lactam |
| 47 | AM; A/S; AUG | lactam | A/S | lactam |
| 48 | AM; A/S; T/S | lactam; other | AM | lactam |
| 49 | AM; A/S; T/S | lactam; other | T/S | other |
| 50 | AM; A/S; AUG | lactam | AM | lactam |

TABLE 22c
Data for Shigella species

| No . | num_lactam | num_other | AM_pv_adj | A/S_pv_adj | AUG_pv_adj | T/S_pv_adj |
|---|---|---|---|---|---|---|
| 1 | 3 | 1 | 2.50E−24 | 2.78E−27 | 6.87E−56 | 6.43E−03 |
| 2 | 3 | 1 | 3.96E−18 | 5.18E−21 | 4.15E−46 | 3.89E−03 |
| 3 | 3 | 1 | 1.61E−18 | 1.80E−21 | 4.15E−46 | 2.75E−03 |
| 4 | 3 | 1 | 1.48E−18 | 1.80E−21 | 1.24E−44 | 5.45E−03 |
| 5 | 3 | 0 | 6.38E−20 | 1.76E−12 | 5.70E−37 | 1.36E−02 |
| 6 | 0 | 1 | 4.19E−01 | 7.59E−01 | 1.18E−01 | 3.04E−34 |
| 7 | 0 | 1 | 4.19E−01 | 7.59E−01 | 1.18E−01 | 3.04E−34 |
| 8 | 0 | 1 | 4.14E−01 | 6.88E−01 | 1.42E−01 | 5.68E−34 |
| 9 | 0 | 1 | 5.86E−01 | 9.62E−01 | 1.89E−01 | 1.47E−26 |
| 10 | 0 | 1 | 6.64E−01 | 8.98E−01 | 3.47E−01 | 4.98E−26 |
| 11 | 3 | 1 | 1.30E−12 | 6.35E−09 | 1.31E−24 | 1.10E−04 |
| 12 | 3 | 1 | 8.43E−13 | 4.07E−09 | 1.67E−24 | 3.04E−05 |
| 13 | 2 | 1 | 5.59E−02 | 5.39E−03 | 8.83E−03 | 3.08E−24 |
| 14 | 3 | 1 | 6.77E−13 | 3.67E−09 | 3.45E−24 | 4.57E−05 |
| 15 | 3 | 1 | 1.11E−11 | 1.11E−08 | 4.84E−23 | 3.29E−12 |
| 16 | 0 | 1 | 7.13E−01 | 9.56E−01 | 8.48E−02 | 1.60E−17 |
| 17 | 3 | 0 | 7.73E−05 | 2.34E−06 | 5.88E−17 | 9.61E−01 |
| 18 | 0 | 1 | 5.80E−01 | 2.16E−01 | 2.14E−01 | 6.18E−13 |
| 19 | 3 | 0 | 5.45E−03 | 1.12E−03 | 6.40E−12 | 1.67E−01 |
| 20 | 3 | 1 | 2.16E−11 | 8.03E−10 | 4.80E−05 | 1.95E−03 |
| 21 | 3 | 0 | 2.93E−08 | 4.44E−11 | 1.86E−03 | 1.59E−01 |
| 22 | 0 | 1 | 1.01E−01 | 3.04E−02 | 5.99E−01 | 2.06E−10 |
| 23 | 0 | 1 | 6.37E−01 | 2.87E−01 | 2.49E−01 | 4.04E−10 |
| 24 | 2 | 1 | 1.17E−08 | 4.60E−05 | 3.74E−02 | 7.92E−04 |
| 25 | 2 | 1 | 1.75E−08 | 3.59E−04 | 9.59E−02 | 5.74E−05 |
| 26 | 3 | 0 | 2.09E−08 | 6.13E−08 | 3.86E−05 | 4.21E−01 |
| 27 | 2 | 1 | 3.60E−08 | 2.62E−04 | 7.92E−02 | 9.64E−04 |
| 28 | 2 | 1 | 3.60E−08 | 5.37E−04 | 7.92E−02 | 1.59E−04 |
| 29 | 2 | 1 | 3.60E−08 | 2.62E−04 | 7.92E−02 | 9.64E−04 |
| 30 | 2 | 1 | 3.60E−08 | 5.37E−04 | 7.92E−02 | 1.59E−04 |
| 31 | 3 | 0 | 6.06E −04 | 1.15E−05 | 3.60E−08 | 1.18E−02 |
| 32 | 3 | 0 | 4.22E−08 | 6.96E−07 | 1.35E−04 | 3.01E−02 |
| 33 | 2 | 1 | 2.70E−07 | 5.88E−08 | 1.78E−01 | 7.31E−04 |
| 34 | 2 | 1 | 2.70E−07 | 5.88E−08 | 1.78E−01 | 7.31E−04 |
| 35 | 2 | 1 | 2.70E−07 | 5.88E−08 | 1.78E−01 | 7.31E−04 |
| 36 | 2 | 1 | 2.70E−07 | 5.88E−08 | 1.78E−01 | 7.31E−04 |
| 37 | 2 | 1 | 2.70E−07 | 5.88E−08 | 1.78E−01 | 7.31E−04 |
| 38 | 3 | 1 | 3.43E−04 | 1.83E−03 | 7.05E−04 | 6.13E−08 |
| 39 | 3 | 1 | 8.13E−05 | 5.17E−04 | 3.23E−04 | 7.77E−08 |
| 40 | 3 | 1 | 2.18E−04 | 3.33E−06 | 8.81E−08 | 1.34E−03 |
| 41 | 3 | 0 | 5.77E−06 | 1.19E−07 | 6.46E−03 | 3.28E−01 |
| 42 | 2 | 1 | 1.50E−07 | 1.17E−03 | 6.39E−02 | 1.05E−04 |
| 43 | 3 | 0 | 1.24E−05 | 2.04E−07 | 6.60E−03 | 3.27E−01 |
| 44 | 3 | 0 | 1.24E−05 | 2.04E−07 | 6.60E−03 | 3.27E−01 |
| 45 | 3 | 0 | 2.09E−07 | 1.92E−06 | 4.75E−05 | 4.59E−01 |
| 46 | 3 | 1 | 1.84E−04 | 2.55E−07 | 1.33E−06 | 3.22E−03 |
| 47 | 3 | 0 | 1.32E−05 | 3.83E−07 | 8.66E−03 | 2.79E−01 |
| 48 | 2 | 1 | 3.98E−07 | 1.01E−05 | 1.08E−01 | 3.36E−03 |
| 49 | 2 | 1 | 3.82E−05 | 1.71E−03 | 2.98E−01 | 4.00E−07 |
| 50 | 3 | 0 | 4.18E−07 | 3.44E−06 | 2.44E−03 | 1.37E−01 |

TABLE 22d
Data for Shigella species

| No. | Locus.Tag | Gene.Symbol | GenBank.Accession | Chromosome |
|---|---|---|---|---|
| 1 | HMPREF9547_01191 | | ZP_07167682 | |
| 2 | HMPREF9551_04221 | | ZP_07191576 | |
| 3 | HMPREF9347_01086 | | ZP_07208640 | |
| 4 | STMDT12_C39170 | | BAJ38860 | |
| 5 | SFxv_1168 | | ADA73425 | |
| 6 | Sdys1_01000012 | | ZP_00922750 | |
| 7 | HMPREF9547_02735 | | ZP_07169192 | |
| 8 | Sdys1_01000013 | | ZP_00922751 | |
| 9 | SFxv_4146 | | ADA76106 | |
| 10 | HMPREF9547_04432 | | ZP_07170853 | |
| 11 | HMPREF9547_00721 | | ZP_07167228 | |
| 12 | SFxv_1141 | | ADA73399 | |
| 13 | ETAE_p041 | folP | YP_003297635 | plasmid pEIB202 |
| 14 | ESCAB7627_0644 | | ZP_02900823 | |
| 15 | SSON_3896 | aadA | YP_312670 | |
| 16 | SFxv_4148 | | ADA76108 | |
| 17 | ESCAB7627_0650 | | ZP_02900555 | |
| 18 | pEFER_0049 | | YP_002394596 | plasmid pEFER |
| 19 | SD1617_4863 | | ZP_07682961 | |
| 20 | ECED1_1050 | | YP_002397074 | |
| 21 | ESCCO14588_3315 | | ZP_03445386 | |
| 22 | ECS88_2899 | rusA | YP_002392541 | |
| 23 | ETAE_p040 | | YP_003297634 | plasmid pEIB202 |
| 24 | EcolE2_01002516 | | ZP_00729149 | |
| 25 | KP1_1529 | betB | YP_002918349 | |
| 26 | EFER_2751 | fhiA | YP_002383857 | |
| 27 | EFER_1964 | | YP_002383093 | |
| 28 | ECEG_03397 | | ZP_06652297 | |
| 29 | HMPREF9534_04860 | | ZP_07189724 | |
| 30 | SFV_0325 | betT | YP_687904 | |
| 31 | SFV_3136 | tsaC | YP_690497 | |
| 32 | SeSA_C0004 | | YP_002113008 | plasmid |
| | | | | pCVM19633_4 |
| 33 | CIT292_00036 | | ZP_03834370 | |
| 34 | EFER_4022 | phoU | YP_002385049 | |
| 35 | SD1617_0888 | pstA | ZP_07679073 | |
| 36 | SbBS512_E4201 | pstC | YP_001882442 | |
| 37 | EFER_4026 | pstS | YP_002385053 | |
| 38 | Senteenterica_010100025269 | | ZP_02664569 | |
| 39 | SSON_PA02 | tetA | YP_001139953 | plasmid |
| | | | | pSS046_spA |
| 40 | EFER_4455 | | YP_002385453 | |
| 41 | ESCAB7627_0589 | agaF | ZP_02900787 | |
| 42 | ECABU_c04070 | betA | ADN45010 | |
| 43 | EcDH1_0571 | | ACX38258 | |
| 44 | S3383 | agaV | NP_838641 | |
| 45 | Ecol5_01002315 | | ZP_00735973 | |
| 46 | EC236275_0400 | | ZP_07778877 | |
| 47 | ESCAB7627_0593 | | ZP_02900381 | |
| 48 | HMPREF9530_03165 | | ZP_07153036 | |
| 49 | SbBS512_E1297 | | YP_001879929 | |
| 50 | CSAG_02340 | | ZP_04563010 | |

TABLE 22e
Data for Shigella species

| No. | Start.Coord | End.Coord | Strand | Scaffold.ID |
|---|---|---|---|---|
| 1 | 6987 | 7817 | + | 648291751 |
| 2 | 10102 | 10662 | − | 648292991 |
| 3 | 2726 | 5692 | − | 648294279 |
| 4 | 4067283 | 4067942 | + | 651053007 |
| 5 | 1128245 | 1129249 | + | 646862363 |
| 6 | 12375 | 12701 | − | 638365528 |
| 7 | 4692 | 5189 | − | 648291885 |
| 8 | 14252 | 14776 | + | 638365528 |
| 9 | 3950954 | 3952570 | − | 646862363 |
| 10 | 8898 | 9719 | − | 648292023 |
| 11 | 5423 | 6016 | − | 648291712 |
| 12 | 1107342 | 1107968 | + | 646862363 |
| 13 | 32101 | 32916 | − | 646311959 |
| 14 | 233492 | 234697 | − | 641784575 |
| 15 | 4110458 | 4111246 | − | 640427102 |
| 16 | 3954100 | 3955767 | − | 646862363 |
| 17 | 238788 | 239996 | − | 641784575 |
| 18 | 48658 | 49503 | + | 643692014 |
| 19 | 80678 | 81835 | − | 649994698 |
| 20 | 1066134 | 1066832 | + | 643348624 |
| 21 | 383590 | 384330 | − | 642987534 |
| 22 | 2863316 | 2863705 | − | 644736332 |
| 23 | 31237 | 32040 | − | 646311959 |
| 24 | 35676 | 36281 | − | 638358744 |
| 25 | 1469088 | 1470560 | − | 646564530 |
| 26 | 2808563 | 2810692 | + | 643692013 |
| 27 | 2010341 | 2013862 | − | 643692013 |
| 28 | 167911 | 168516 | − | 647536674 |
| 29 | 4513 | 7977 | − | 648289846 |
| 30 | 342056 | 344089 | + | 637000590 |
| 31 | 3209986 | 3212835 | − | 637000590 |
| 32 | 2398 | 3933 | − | 642555220 |
| 33 | 7614 | 8387 | + | 643891493 |
| 34 | 4127070 | 4127795 | − | 643692013 |
| 35 | 217087 | 217971 | − | 649994663 |
| 36 | 3927571 | 3928530 | + | 641522725 |
| 37 | 4130610 | 4131650 | − | 643692013 |
| 38 | 35858 | 36535 | − | 641777693 |
| 39 | 1155 | 2429 | + | 640427123 |
| 40 | 4539896 | 4540645 | + | 643692013 |
| 41 | 182707 | 183141 | − | 641784575 |
| 42 | 425884 | 427554 | − | 648231700 |
| 43 | 602662 | 603063 | − | 646862383 |
| 44 | 3261857 | 3262366 | + | 637000219 |
| 45 | 39234 | 40157 | − | 638359205 |
| 46 | 26968 | 27516 | − | 649994608 |
| 47 | 185313 | 186593 | − | 641784575 |
| 48 | 5665 | 6162 | − | 648290513 |
| 49 | 1184368 | 1184799 | + | 641522725 |
| 50 | 2538496 | 2539857 | + | 646206732 |

### Example 12

### Staphylococcus, particularly S. aureus

The procedure was carried out as in Example 1, except for the following changes:

Bacterial Strains

The inventors selected 1001 specimens of S. aureus from the microbiology strain collection at Siemens Healthcare Diagnostics (West Sacramento, Calif.) for susceptibility testing and whole genome sequencing, of which 985 had an assembly, a unique Kiel NGS ID (NGS data and assembly ID, a unique resistance profile (no different resistance profiles with different outcomes, and at least one drug with non-missing resistance value, so that these were further analyzed.

From MetaRef, 754 centroids of S. aureus were used as reference sequences.

Resistance/susceptibility was determined for the following antibiotics as described below: Amoxicillin/Clavulanate, Ampicillin, Ampicillin/Sulbactam, Azithromycin, Cefalothin, Cefazolin, Cefepime, Cefotaxime, Cefoxitin, Ceftriaxone, Cefuroxime, Chloramphenicol, Ciprofloxacin, Clindamycin, Daptomycin, Ertapenem, Erythromycin, Fosfomycin, Fusidic acid, Gentamicin, Imipenem, Levofloxacin, Linezolid, Meropenem, Moxifloxacin, Mupirocin, Nitrofurantoin, Norfloxacin, Ofloxacin, Oxacillin, Penicillin G, Piperacillin/Tazobactam, Quinupristin/Dalfopristin, Rifampicin, Teicoplanin, Tetracycline, Tigecycline, Tobramycin, Trimethoprim/Sulfamethoxazole, and Vancomycin.

For testing, standard procedures were used, i.e. VITEK 2 system and AST cards (Biomerieux), Microscan system and AST panels (Beckmann Coulter).

DNA Extraction

DNA extraction and purification was carried out using the MagAttract HMW DNA Kit (Qiagen) procedure with the following changes. After up to 2×10⁹ bacteria (1m1 culture) were centrifuged in a 2 ml tube (10 min, 5000 ×g) and the supernatant was discharged, it was again centrifuged 1 min and the sample was taken. The resulting pellet was dispersed in 160 μl P1, 20 μl lysozyme (100 mg/ml) and 4 μl lysostaphin were added and mixed, and the suspension was incubated at 37° C. at 900 rpm for 30 mins in a thermal mixer. Afterwards 300 μl lysis buffer and proteinase K (30 μl) (both from the blood kit for Maxwell of Promega) were added and the whole again incubated for 30 mins at 56° C. and 900 rpm. The samples as a whole (˜510 μl) were then transferred to the Maxwell cartridges for further processing, using the Tissue LEV Total RNA Kit AS1220 or the XAS1220 Custom Kit (Promega).

Next generation sequencing and data analysis was carried out as in Example 1.

The results for S. aureus are shown in Tables 23 (corresponding to Table 1) and 24 (corresponding to Table 2).

TABLE 23a
Data for Staphylococcus, particularly S. aureus

| No. | Scaffold.External.Accession | Scaffold.Name | best_pv |
|---|---|---|---|
| 1 | NZ_ACJC01000132 | Staphylococcus epidermidis W23144: NZ_ACJC01000132 | 9.38479881072327e−227 |
| 2 | NZ_ADMU01000036 | Staphylococcus epidermidis M23864: W2 (grey) contig00037: | 9.38479881072327e−227 |
| | | NZ_ADMU01000036 | |
| 3 | NZ_ADMU01000036 | Staphylococcus epidermidis M23864: W2 (grey) contig00037: | 2.67435403500704e−225 |
| | | NZ_ADMU01000036 | |
| 4 | NZ_GG770515 | Staphylococcus aureus subsp. aureus EMRSA16 genomic scaffold | 1.77186061517335e−211 |
| | | supercont1.3: NZ_GG770515 | |
| 5 | NZ_ACHE01000064 | Staphylococcus epidermidis BCM-HMP0060: NZ_ACHE01000064 | 3.22333061264623e−210 |
| 6 | NZ_ACKJ01000008 | Staphylococcus aureus A9719: NZ_ACKJ01000008 | 4.28801157250818e−188 |
| 7 | FN433596 | Staphylococcus aureus subsp. aureus TW20: FN433596 | 6.72085159673615e−181 |
| 8 | FN433596 | Staphylococcus aureus subsp. aureus TW20: FN433596 | 6.72085159673615e−181 |
| 9 | NC_002976 | Staphylococcus epidermidis RP62A: NC_002976 | 6.72085159673615e−181 |
| 10 | NZ_ACKF01000035 | Staphylococcus aureus A6300: NZ_ACKF01000035 | 8.5426103876016e−179 |
| 11 | NC_002976 | Staphylococcus epidermidis RP62A: NC_002976 | 8.5426103876016e−179 |
| 12 | NC_002976 | Staphylococcus epidermidis RP62A: NC_002976 | 1.81714784068785e−175 |
| 13 | NZ_ADMU01000035 | Staphylococcus epidermidis M23864: W2 (grey) contig00036: | 3.23410033711171e−175 |
| | | NZ_ADMU01000035 | |
| 14 | NC_002976 | Staphylococcus epidermidis RP62A: NC_002976 | 9.12700235375297e−175 |
| 15 | NC_002976 | Staphylococcus epidermidis RP62A: NC_002976 | 9.12700235375297e−175 |
| 16 | NC_002976 | Staphylococcus epidermidis RP62A: NC_002976 | 9.12700235375297e−175 |
| 17 | NC_002976 | Staphylococcus epidermidis RP62A: NC_002976 | 9.12700235375297e−175 |
| 18 | NC_002976 | Staphylococcus epidermidis RP62A: NC_002976 | 2.25944344923286e−173 |
| 19 | NZ_ACKF01000007 | Staphylococcus aureus A6300: NZ_ACKF01000007 | 2.40063417489507e−172 |
| 20 | NC_002976 | Staphylococcus epidermidis RP62A: NC_002976 | 2.59802993040457e−167 |
| 21 | NC_002976 | Staphylococcus epidermidis RP62A: NC_002976 | 3.12953724144125e−166 |
| 22 | NZ_ADJJ01000031 | Staphylococcus aureus A8796 cont1.31: NZ_ADJJ01000031 | 3.37208086862656e−166 |
| 23 | NC_002745 | Staphylococcus aureus subsp. aureus N315: NC_002745 | 3.37208086862656e−166 |
| 24 | NC_002976 | Staphylococcus epidermidis RP62A: NC_002976 | 7.17319474297637e−161 |
| 25 | NZ_ACHH01000128 | Staphylococcus aureus subsp. aureus TCH70: NZ_ACHH01000128 | 4.16856451727179e−152 |
| 26 | NZ_ACKC01000026 | Staphylococcus aureus A5937: NZ_ACKC01000026 | 1.58751373713434e−135 |
| 27 | NZ_ACKC01000026 | Staphylococcus aureus A5937: NZ_ACKC01000026 | 1.69633422486642e−135 |
| 28 | CP001844 | Staphylococcus aureus 04-02981: CP001844 | 1.77656315889944e−134 |
| 29 | FR714927 | Staphylococcus aureus subsp. aureus ECT-R 2: FR714927 | 4.06264970457926e−133 |
| 30 | CP001844 | Staphylococcus aureus 04-02981: CP001844 | 1.01251954267327e−130 |
| 31 | NC_007622 | Staphylococcus aureus RF122: NC_007622 | 4.84169534955158e−126 |
| 32 | NC_009632 | Staphylococcus aureus subsp. aureus JH1: NC_009632 | 6.20485118760467e−122 |
| 33 | NC_002976 | Staphylococcus epidermidis RP62A: NC_002976 | 5.75915090813908e−120 |
| 34 | NZ_ACYO01000032 | Staphylococcus aureus A8117 cont1.32: NZ_ACYO01000032 | 1.84672228386398e−116 |
| 35 | FR714927 | Staphylococcus aureus subsp. aureus ECT-R 2: FR714927 | 7.1946440374743e−116 |
| 36 | NZ_ACHD01000273 | Staphylococcus aureus subsp. aureus TCH130: NZ_ACHD01000273 | 4.22320321058421e−113 |
| 37 | NZ_ACKE01000012 | Staphylococcus aureus A6224: NZ_ACKE01000012 | 4.22320321058421e−113 |
| 38 | CP002114 | Staphylococcus aureus subsp. aureus JKD6159: CP002114 | 4.83609713640718e−112 |
| 39 | NZ_GG730131 | Staphylococcus aureus subsp. aureus C101 genomic scaffold | 4.63942446669448e−110 |
| | | supercont1.12: NZ_GG730131 | |
| 40 | NZ_ACKF01000014 | Staphylococcus aureus A6300: NZ_ACKF01000014 | 5.76090691723747e−108 |
| 41 | NZ_ACKK01000039 | Staphylococcus aureus A9763: NZ_ACKK01000039 | 2.40583396327414e−107 |
| 42 | NZ_ACKE01000008 | Staphylococcus aureus A6224: NZ_ACKE01000008 | 2.99974766558167e−106 |
| 43 | NZ_ACKG01000031 | Staphylococcus aureus A8115: NZ_ACKG01000031 | 1.57559858677166e−102 |
| 44 | NC_009632 | Staphylococcus aureus subsp. aureus JH1: NC_009632 | 1.0725409335823e−101 |
| 45 | NZ_ACYO01000003 | Staphylococcus aureus A8117 cont1.3: NZ_ACYO01000003 | 1.0725409335823e−101 |
| 46 | FR714929 | Staphylococcus aureus subsp. aureus ECT-R 2 plasmid | 5.26123840198017e−101 |
| | | pLUH02: FR714929 | |
| 47 | CP001844 | Staphylococcus aureus 04-02981: CP001844 | 8.01600909282956e−101 |
| 48 | CP001844 | Staphylococcus aureus 04-02981: CP001844 | 1.78546333248495e−95 |
| 49 | NZ_ADJK01000020 | Staphylococcus aureus A8819 cont1.20: NZ_ADJK01000020 | 1.80469278143707e−94 |
| 50 | NZ_GL545262 | Staphylococcus hominis subsp. hominis C80 genomic scaffold | 2.82728623419544e−94 |
| | | supercont1.11: NZ_GL545262 | |

TABLE 23b
Data for Staphylococcus, particularly S. aureus (continued)

| No. | sign_phenos |
|---|---|
| 1 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 2 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 3 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 4 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 5 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 6 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 7 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 8 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 9 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 10 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 11 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 12 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 13 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 14 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 15 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 16 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 17 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 18 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 19 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 20 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 21 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 22 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 23 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 24 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 25 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 26 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 27 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 28 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 29 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 30 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 31 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 32 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tobramycin |
| 33 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 34 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tobramycin |
| 35 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 36 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 37 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 38 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 39 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 40 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 41 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 42 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 43 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 44 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 45 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 46 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 47 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 48 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 49 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 50 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |

TABLE 23c
Data for Staphylococcus, particularly S. aureus (continued)

| No. | sign_phenos_class | best_pheno | best_pheno_class |
|---|---|---|---|
| 1 | aminoglycoside; fluoroquinolone; lactam; lincosamide; macrolide | Oxacillin | lactam |
| 2 | aminoglycoside; fluoroquinolone; lactam; lincosamide; macrolide | Oxacillin | lactam |
| 3 | aminoglycoside; fluoroquinolone; lactam; lincosamide; macrolide | Oxacillin | lactam |
| 4 | aminoglycoside; fluoroquinolone; lactam; lincosamide; macrolide | Oxacillin | lactam |
| 5 | aminoglycoside; fluoroquinolone; lactam; lincosamide; macrolide | Oxacillin | lactam |
| 6 | aminoglycoside; fluoroquinolone; lactam; lincosamide; macrolide | Moxifloxacin | fluoroquinolone |
| 7 | aminoglycoside; fluoroquinolone; lactam; lincosamide; macrolide; tetracycline | Oxacillin | lactam |
| 8 | aminoglycoside; fluoroquinolone; lactam; lincosamide; macrolide; tetracycline | Oxacillin | lactam |
| 9 | aminoglycoside; fluoroquinolone; lactam; lincosamide; macrolide; tetracycline | Oxacillin | lactam |
| 10 | aminoglycoside; fluoroquinolone; lactam; lincosamide; macrolide; tetracycline | Oxacillin | lactam |
| 11 | aminoglycoside; fluoroquinolone; lactam; lincosamide; macrolide; tetracycline | Oxacillin | lactam |
| 12 | aminoglycoside; fluoroquinolone; lactam; lincosamide; macrolide; tetracycline | Oxacillin | lactam |
| 13 | aminoglycoside; fluoroquinolone; lactam; lincosamide; macrolide; tetracycline | Oxacillin | lactam |
| 14 | aminoglycoside; fluoroquinolone; lactam; lincosamide; macrolide; tetracycline | Oxacillin | lactam |
| 15 | aminoglycoside; fluoroquinolone; lactam; lincosamide; macrolide; tetracycline | Oxacillin | lactam |
| 16 | aminoglycoside; fluoroquinolone; lactam; lincosamide; macrolide; tetracycline | Oxacillin | lactam |
| 17 | aminoglycoside; fluoroquinolone; lactam; lincosamide; macrolide; tetracycline | Oxacillin | lactam |
| 18 | aminoglycoside; fluoroquinolone; lactam; lincosamide; macrolide; tetracycline | Oxacillin | lactam |
| 19 | aminoglycoside; fluoroquinolone; lactam; lincosamide; macrolide; tetracycline | Oxacillin | lactam |
| 20 | aminoglycoside; fluoroquinolone; lactam; lincosamide; macrolide; tetracycline | Moxifloxacin | fluoroquinolone |
| 21 | aminoglycoside; fluoroquinolone; lactam; lincosamide; macrolide; tetracycline | Moxifloxacin | fluoroquinolone |
| 22 | aminoglycoside; fluoroquinolone; lactam; lincosamide; macrolide; tetracycline | Moxifloxacin | fluoroquinolone |
| 23 | aminoglycoside; fluoroquinolone; lactam; lincosamide; macrolide; tetracycline | Moxifloxacin | fluoroquinolone |
| 24 | aminoglycoside; fluoroquinolone; lactam; lincosamide; macrolide; tetracycline | Moxifloxacin | fluoroquinolone |
| 25 | aminoglycoside; fluoroquinolone; lactam; lincosamide; macrolide; tetracycline | Moxifloxacin | fluoroquinolone |
| 26 | aminoglycoside; fluoroquinolone; lactam; lincosamide; macrolide; tetracycline | Moxifloxacin | fluoroquinolone |
| 27 | aminoglycoside; fluoroquinolone; lactam; lincosamide; macrolide; tetracycline | Moxifloxacin | fluoroquinolone |
| 28 | aminoglycoside; fluoroquinolone; lactam; lincosamide; macrolide; tetracycline | Moxifloxacin | fluoroquinolone |
| 29 | aminoglycoside; fluoroquinolone; lactam; lincosamide; macrolide; tetracycline | Moxifloxacin | fluoroquinolone |
| 30 | aminoglycoside; fluoroquinolone; lactam; lincosamide; macrolide; tetracycline | Moxifloxacin | fluoroquinolone |
| 31 | aminoglycoside; fluoroquinolone; lactam; lincosamide; macrolide; tetracycline | Moxifloxacin | fluoroquinolone |
| 32 | aminoglycoside; fluoroquinolone; lactam; lincosamide; macrolide | Moxifloxacin | fluoroquinolone |
| 33 | aminoglycoside; fluoroquinolone; lactam; lincosamide; macrolide; tetracycline | Moxifloxacin | fluoroquinolone |
| 34 | aminoglycoside; fluoroquinolone; lactam; lincosamide; macrolide | Moxifloxacin | fluoroquinolone |
| 35 | aminoglycoside; fluoroquinolone; lactam; lincosamide; macrolide; tetracycline | Moxifloxacin | fluoroquinolone |
| 36 | aminoglycoside; fluoroquinolone; lactam; lincosamide; macrolide; tetracycline | Moxifloxacin | fluoroquinolone |
| 37 | aminoglycoside; fluoroquinolone; lactam; lincosamide; macrolide; tetracycline | Moxifloxacin | fluoroquinolone |
| 38 | aminoglycoside; fluoroquinolone; lactam; lincosamide; macrolide; tetracycline | Moxifloxacin | fluoroquinolone |
| 39 | aminoglycoside; fluoroquinolone; lactam; lincosamide; macrolide; tetracycline | Moxifloxacin | fluoroquinolone |
| 40 | aminoglycoside; fluoroquinolone; lactam; lincosamide; macrolide; tetracycline | Moxifloxacin | fluoroquinolone |
| 41 | aminoglycoside; fluoroquinolone; lactam; lincosamide; macrolide; tetracycline | Moxifloxacin | fluoroquinolone |
| 42 | aminoglycoside; fluoroquinolone; lactam; lincosamide; macrolide; tetracycline | Moxifloxacin | fluoroquinolone |
| 43 | aminoglycoside; fluoroquinolone; lactam; lincosamide; macrolide; tetracycline | Moxifloxacin | fluoroquinolone |
| 44 | aminoglycoside; fluoroquinolone; lactam; lincosamide; macrolide; tetracycline | Moxifloxacin | fluoroquinolone |
| 45 | aminoglycoside; fluoroquinolone; lactam; lincosamide; macrolide; tetracycline | Moxifloxacin | fluoroquinolone |
| 46 | aminoglycoside; fluoroquinolone; lactam; lincosamide; macrolide; tetracycline | Moxifloxacin | fluoroquinolone |
| 47 | aminoglycoside; fluoroquinolone; lactam; lincosamide; macrolide; tetracycline | Moxifloxacin | fluoroquinolone |
| 48 | aminoglycoside; fluoroquinolone; lactam; lincosamide; macrolide; tetracycline | Moxifloxacin | fluoroquinolone |
| 49 | aminoglycoside; fluoroquinolone; lactam; lincosamide; macrolide; tetracycline | Moxifloxacin | fluoroquinolone |
| 50 | aminoglycoside; fluoroquinolone; lactam; lincosamide; macrolide; tetracycline | Oxacillin | lactam |

TABLE 23d
Data for Staphylococcus, particularly S. aureus (continued)

| No. | num_aminoglycoside | num_fluoroquinolone | num_lactam | num_lincosamide | num_macrolide | num_tetracycline |
|---|---|---|---|---|---|---|
| 1 | 1 | 3 | 9 | 1 | 1 | 0 |
| 2 | 1 | 3 | 9 | 1 | 1 | 0 |
| 3 | 1 | 3 | 9 | 1 | 1 | 0 |
| 4 | 1 | 3 | 9 | 1 | 1 | 0 |
| 5 | 1 | 3 | 9 | 1 | 1 | 0 |
| 6 | 1 | 3 | 9 | 1 | 1 | 0 |
| 7 | 1 | 3 | 9 | 1 | 1 | 1 |
| 8 | 1 | 3 | 9 | 1 | 1 | 1 |
| 9 | 1 | 3 | 9 | 1 | 1 | 1 |
| 10 | 1 | 3 | 9 | 1 | 1 | 1 |
| 11 | 1 | 3 | 9 | 1 | 1 | 1 |
| 12 | 1 | 3 | 9 | 1 | 1 | 1 |
| 13 | 1 | 3 | 9 | 1 | 1 | 1 |
| 14 | 1 | 3 | 9 | 1 | 1 | 1 |
| 15 | 1 | 3 | 9 | 1 | 1 | 1 |
| 16 | 1 | 3 | 9 | 1 | 1 | 1 |
| 17 | 1 | 3 | 9 | 1 | 1 | 1 |
| 18 | 1 | 3 | 9 | 1 | 1 | 1 |
| 19 | 1 | 3 | 9 | 1 | 1 | 1 |
| 20 | 1 | 3 | 9 | 1 | 1 | 1 |
| 21 | 1 | 3 | 9 | 1 | 1 | 1 |
| 22 | 1 | 3 | 9 | 1 | 1 | 1 |
| 23 | 1 | 3 | 9 | 1 | 1 | 1 |
| 24 | 1 | 3 | 9 | 1 | 1 | 1 |
| 25 | 1 | 3 | 9 | 1 | 1 | 1 |
| 26 | 1 | 3 | 8 | 1 | 1 | 1 |
| 27 | 1 | 3 | 8 | 1 | 1 | 1 |
| 28 | 1 | 3 | 8 | 1 | 1 | 1 |
| 29 | 1 | 3 | 8 | 1 | 1 | 1 |
| 30 | 1 | 3 | 8 | 1 | 1 | 1 |
| 31 | 1 | 3 | 9 | 1 | 1 | 1 |
| 32 | 1 | 3 | 8 | 1 | 1 | 0 |
| 33 | 1 | 3 | 9 | 1 | 1 | 1 |
| 34 | 1 | 3 | 8 | 1 | 1 | 0 |
| 35 | 1 | 3 | 8 | 1 | 1 | 1 |
| 36 | 1 | 3 | 9 | 1 | 1 | 1 |
| 37 | 1 | 3 | 9 | 1 | 1 | 1 |
| 38 | 1 | 3 | 8 | 1 | 1 | 1 |
| 39 | 1 | 3 | 8 | 1 | 1 | 1 |
| 40 | 1 | 3 | 8 | 1 | 1 | 1 |
| 41 | 1 | 3 | 8 | 1 | 1 | 1 |
| 42 | 1 | 3 | 8 | 1 | 1 | 1 |
| 43 | 1 | 3 | 8 | 1 | 1 | 1 |
| 44 | 1 | 3 | 8 | 1 | 1 | 1 |
| 45 | 1 | 3 | 8 | 1 | 1 | 1 |
| 46 | 1 | 3 | 9 | 1 | 1 | 1 |
| 47 | 1 | 3 | 8 | 1 | 1 | 1 |
| 48 | 1 | 3 | 8 | 1 | 1 | 1 |
| 49 | 1 | 3 | 9 | 1 | 1 | 1 |
| 50 | 1 | 3 | 9 | 1 | 1 | 1 |

TABLE 23e
Data for Staphylococcus, particularly S. aureus (continued)

| No. | Ampicillin_pv_adj | Ampicillin/Sulbactam_pv_adj | Cefepim_pv_adj | Cefotaxim_pv_adj |
|---|---|---|---|---|
| 1 | 2.20E−08 | 5.96E−37 | 5.96E−37 | 5.96E−37 |
| 2 | 2.20E−08 | 5.96E−37 | 5.96E−37 | 5.96E−37 |
| 3 | 2.20E−08 | 5.96E−37 | 5.96E−37 | 5.96E−37 |
| 4 | 4.64E−08 | 4.55E−35 | 4.55E−35 | 4.55E−35 |
| 5 | 2.20E−08 | 2.16E−33 | 2.16E−33 | 2.16E−33 |
| 6 | 2.22E−09 | 1.64E−31 | 1.64E−31 | 1.64E−31 |
| 7 | 3.22E−07 | 2.72E−27 | 2.72E−27 | 2.72E−27 |
| 8 | 3.22E−07 | 2.72E−27 | 2.72E−27 | 2.72E−27 |
| 9 | 3.22E−07 | 2.72E−27 | 2.72E−27 | 2.72E−27 |
| 10 | 3.22E−07 | 2.72E−27 | 2.72E−27 | 2.72E−27 |
| 11 | 3.22E−07 | 2.72E−27 | 2.72E−27 | 2.72E−27 |
| 12 | 6.85E−07 | 2.72E−27 | 2.72E−27 | 2.72E−27 |
| 13 | 2.90E−07 | 1.05E−25 | 1.05E−25 | 1.05E−25 |
| 14 | 3.22E−07 | 2.72E−27 | 2.72E−27 | 2.72E−27 |
| 15 | 3.22E−07 | 2.72E−27 | 2.72E−27 | 2.72E−27 |
| 16 | 3.22E−07 | 2.72E−27 | 2.72E−27 | 2.72E−27 |
| 17 | 3.22E−07 | 2.72E−27 | 2.72E−27 | 2.72E−27 |
| 18 | 2.90E−07 | 1.05E−25 | 1.05E−25 | 1.05E−25 |
| 19 | 2.90E−07 | 1.05E−25 | 1.05E−25 | 1.05E−25 |
| 20 | 8.65E−04 | 1.18E−21 | 1.18E−21 | 1.18E−21 |
| 21 | 8.65E−04 | 1.18E−21 | 1.18E−21 | 1.18E−21 |
| 22 | 3.21E−03 | 7.21E−21 | 7.21E−21 | 7.21E−21 |
| 23 | 3.21E−03 | 7.21E−21 | 7.21E−21 | 7.21E−21 |
| 24 | 1.55E−03 | 1.27E−19 | 1.27E−19 | 1.27E−19 |
| 25 | 1.83E−03 | 2.01E−19 | 2.01E−19 | 2.01E−19 |
| 26 | 1.26E−02 | 1.32E−20 | 1.32E−20 | 1.32E−20 |
| 27 | 1.18E−02 | 7.81E−20 | 7.81E−20 | 7.81E−20 |
| 28 | 1.18E−02 | 7.81E−20 | 7.81E−20 | 7.81E−20 |
| 29 | 1.26E−02 | 7.25E−19 | 7.25E−19 | 7.25E−19 |
| 30 | 3.28E−02 | 3.76E−18 | 3.76E−18 | 3.76E−18 |
| 31 | 4.05E−03 | 1.35E−15 | 1.35E−15 | 1.35E−15 |
| 32 | 1.24E−02 | 1.13E−19 | 1.13E−19 | 1.13E−19 |
| 33 | 1.11E−03 | 1.73E−15 | 1.73E−15 | 1.73E−15 |
| 34 | 1.27E−02 | 6.35E−19 | 6.35E−19 | 6.35E−19 |
| 35 | 2.32E−02 | 3.05E−13 | 3.05E−13 | 3.05E−13 |
| 36 | 7.10E−03 | 2.03E−18 | 2.03E−18 | 2.03E−18 |
| 37 | 7.10E−03 | 2.03E−18 | 2.03E−18 | 2.03E−18 |
| 38 | 1.24E−02 | 2.91E−15 | 2.91E−15 | 2.91E−15 |
| 39 | 3.37E−02 | 1.60E−16 | 1.60E−16 | 1.60E−16 |
| 40 | 5.70E−02 | 1.88E−12 | 1.88E−12 | 1.88E−12 |
| 41 | 1.15E−01 | 1.25E−15 | 1.25E−15 | 1.25E−15 |
| 42 | 3.84E−02 | 9.78E−11 | 9.78E−11 | 9.78E−11 |
| 43 | 3.56E−02 | 5.77E−17 | 5.77E−17 | 5.77E−17 |
| 44 | 3.37E−02 | 1.60E−16 | 1.60E−16 | 1.60E−16 |
| 45 | 3.37E−02 | 1.60E−16 | 1.60E−16 | 1.60E−16 |
| 46 | 2.90E−07 | 1.61E−15 | 1.61E−15 | 1.61E−15 |
| 47 | 7.69E−02 | 7.81E−20 | 7.81E−20 | 7.81E−20 |
| 48 | 8.07E−02 | 6.08E−12 | 6.08E−12 | 6.08E−12 |
| 49 | 2.90E−07 | 1.61E−15 | 1.61E−15 | 1.61E−15 |
| 50 | 3.04E−06 | 1.82E−17 | 1.82E−17 | 1.82E−17 |

TABLE 23f
Data for Staphylococcus, particularly S. aureus (continued)

| No. | Cefuroxim_pv_adj | Ciprofloxacin_pv_adj | Clindamycin_pv_adj | Erythromycin_pv_adj |
|---|---|---|---|---|
| 1 | 1.02E−172 | 4.82E−131 | 5.41E−111 | 1.04E−111 |
| 2 | 1.02E−172 | 4.82E−131 | 5.41E−111 | 1.04E−111 |
| 3 | 1.02E−172 | 3.02E−132 | 6.31E−112 | 7.39E−113 |
| 4 | 1.19E−170 | 1.00E−131 | 5.45E−114 | 1.72E−111 |
| 5 | 9.55E−169 | 1.65E−131 | 8.55E−113 | 3.44E−110 |
| 6 | 3.09E−155 | 2.03E−145 | 7.78E−102 | 4.54E−100 |
| 7 | 2.00E−140 | 4.08E−138 | 6.00E−125 | 2.44E−127 |
| 8 | 2.00E−140 | 5.95E−135 | 7.70E−123 | 2.44E−127 |
| 9 | 2.00E−140 | 4.08E−138 | 6.00E−125 | 2.44E−127 |
| 10 | 2.29E−138 | 2.11E−136 | 1.31E−123 | 4.65E−126 |
| 11 | 2.29E−138 | 3.02E−133 | 1.67E−121 | 4.65E−126 |
| 12 | 1.01E−138 | 1.36E−128 | 4.01E−120 | 1.01E−123 |
| 13 | 2.71E−145 | 1.43E−135 | 2.49E−122 | 2.94E−121 |
| 14 | 5.87E−142 | 6.84E−140 | 7.88E−125 | 3.61E−124 |
| 15 | 5.87E−142 | 6.84E−140 | 7.88E−125 | 3.61E−124 |
| 16 | 5.87E−142 | 6.84E−140 | 7.88E−125 | 3.61E−124 |
| 17 | 5.87E−142 | 6.84E−140 | 7.88E−125 | 3.61E−124 |
| 18 | 3.15E−140 | 6.84E−140 | 1.65E−123 | 1.01E−122 |
| 19 | 3.78E−139 | 5.24E−139 | 7.90E−123 | 4.58E−122 |
| 20 | 3.99E−125 | 5.65E−132 | 3.88E−136 | 8.83E−140 |
| 21 | 5.82E−123 | 4.09E−130 | 1.24E−134 | 2.39E−138 |
| 22 | 4.30E−124 | 5.65E−132 | 7.67E−139 | 1.30E−142 |
| 23 | 4.30E−124 | 5.65E−132 | 7.67E−139 | 1.30E−142 |
| 24 | 8.70E−122 | 4.09E−130 | 4.42E−135 | 5.01E−139 |
| 25 | 1.95E−112 | 3.48E−117 | 1.09E−121 | 8.26E−126 |
| 26 | 9.99E−102 | 2.00E−99 | 7.42E−94 | 1.88E−93 |
| 27 | 9.92E−101 | 1.25E−100 | 4.84E−95 | 2.65E−94 |
| 28 | 1.08E−99 | 6.62E−99 | 5.06E−95 | 1.80E−94 |
| 29 | 4.45E−100 | 1.46E−101 | 3.05E−98 | 1.82E−99 |
| 30 | 3.62E−99 | 6.76E−98 | 4.11E−89 | 2.04E−88 |
| 31 | 5.46E−82 | 8.46E−90 | 4.95E−69 | 2.56E−67 |
| 32 | 3.45E−93 | 1.68E−88 | 1.37E−90 | 2.27E−88 |
| 33 | 7.10E−92 | 7.29E−86 | 6.57E−78 | 1.16E−76 |
| 34 | 3.46E−90 | 5.20E−83 | 1.49E−80 | 4.89E−80 |
| 35 | 9.56E−73 | 1.94E−83 | 1.78E−63 | 2.45E−61 |
| 36 | 1.95E−83 | 3.50E−91 | 5.92E−79 | 2.14E−84 |
| 37 | 1.95E−83 | 3.50E−91 | 5.92E−79 | 2.14E−84 |
| 38 | 7.71E−76 | 4.08E−77 | 6.05E−58 | 6.49E−57 |
| 39 | 3.93E−89 | 4.20E−78 | 5.89E−71 | 3.43E−72 |
| 40 | 3.54E−69 | 3.19E−77 | 4.43E−58 | 3.86E−57 |
| 41 | 8.95E−84 | 1.49E−77 | 1.71E−74 | 1.87E−74 |
| 42 | 3.77E−75 | 8.95E−84 | 6.90E−85 | 9.85E−84 |
| 43 | 6.75E−81 | 1.60E−81 | 9.46E−72 | 9.56E−79 |
| 44 | 3.58E−81 | 2.83E−81 | 9.63E−72 | 1.15E−78 |
| 45 | 4.14E−80 | 2.83E−81 | 1.29E−70 | 1.77E−77 |
| 46 | 7.59E−62 | 5.15E−79 | 7.94E−76 | 5.98E−76 |
| 47 | 6.11E−79 | 1.32E−73 | 5.43E−98 | 2.20E−97 |
| 48 | 1.16E−77 | 7.87E−68 | 1.31E−67 | 3.92E−67 |
| 49 | 5.08E−59 | 3.55E−74 | 3.25E−71 | 5.25E−71 |
| 50 | 4.13E−78 | 4.35E−52 | 2.56E−53 | 1.24E−51 |

TABLE 23g
Data for Staphylococcus, particularly S. aureus (continued)

| No. | Imipenem_pv_adj | Levofloxacin_pv_adj | Moxifloxacin_pv_adj | Oxacillin_pv_adj |
|---|---|---|---|---|
| 1 | 2.22E−97 | 8.36E−31 | 1.85E−167 | 9.38E−227 |
| 2 | 2.22E−97 | 8.36E−31 | 1.85E−167 | 9.38E−227 |
| 3 | 2.22E−97 | 8.36E−31 | 1.24E−168 | 2.67E−225 |
| 4 | 5.09E−91 | 5.82E−30 | 1.90E−168 | 1.77E−211 |
| 5 | 2.50E−88 | 1.50E−28 | 3.37E−166 | 3.22E−210 |
| 6 | 1.02E−75 | 5.49E−34 | 4.29E−188 | 2.96E−187 |
| 7 | 5.60E−79 | 1.02E−27 | 1.24E−168 | 6.72E−181 |
| 8 | 5.60E−79 | 1.02E−27 | 8.38E−166 | 6.72E−181 |
| 9 | 5.60E−79 | 1.02E−27 | 1.24E−168 | 6.72E−181 |
| 10 | 5.60E−79 | 1.02E−27 | 9.67E−167 | 8.54E−179 |
| 11 | 7.66E−78 | 1.02E−27 | 6.64E−164 | 8.54E−179 |
| 12 | 5.60E−79 | 5.41E−27 | 4.22E−160 | 1.82E−175 |
| 13 | 1.11E−77 | 1.82E−28 | 7.77E−170 | 3.23E−175 |
| 14 | 5.16E−79 | 1.02E−27 | 1.02E−172 | 9.13E−175 |
| 15 | 5.16E−79 | 1.02E−27 | 1.02E−172 | 9.13E−175 |
| 16 | 5.16E−79 | 1.02E−27 | 1.02E−172 | 9.13E−175 |
| 17 | 5.16E−79 | 1.02E−27 | 1.02E−172 | 9.13E−175 |
| 18 | 1.11E−77 | 4.43E−26 | 5.10E−171 | 2.26E−173 |
| 19 | 1.11E−77 | 4.43E−26 | 4.94E−170 | 2.40E−172 |
| 20 | 2.49E−65 | 7.88E−28 | 2.60E−167 | 1.43E−155 |
| 21 | 7.09E−65 | 7.88E−28 | 3.13E−166 | 2.33E−153 |
| 22 | 1.38E−64 | 8.83E−27 | 3.37E−166 | 1.17E−154 |
| 23 | 1.38E−64 | 8.83E−27 | 3.37E−166 | 1.17E−154 |
| 24 | 6.20E−63 | 2.26E−24 | 7.17E−161 | 3.62E−151 |
| 25 | 1.34E−61 | 1.22E−26 | 4.17E−152 | 4.81E−142 |
| 26 | 6.28E−51 | 3.77E−23 | 1.59E−135 | 2.07E−120 |
| 27 | 6.51E−51 | 2.53E−22 | 1.70E−135 | 8.40E−122 |
| 28 | 6.51E−51 | 2.53E−22 | 1.78E−134 | 8.52E−120 |
| 29 | 5.53E−51 | 1.45E−21 | 4.06E−133 | 1.84E−119 |
| 30 | 8.78E−49 | 9.16E−21 | 1.01E−130 | 3.53E−117 |
| 31 | 8.13E−44 | 1.25E−24 | 4.84E−126 | 3.22E−102 |
| 32 | 2.98E−44 | 2.87E−19 | 6.20E−122 | 5.33E−111 |
| 33 | 6.56E−47 | 5.69E−23 | 5.76E−120 | 7.13E−108 |
| 34 | 4.99E−46 | 2.00E−18 | 1.85E−116 | 2.91E−105 |
| 35 | 1.79E−38 | 1.08E−20 | 7.19E−116 | 1.17E−92 |
| 36 | 4.65E−43 | 6.88E−21 | 4.22E−113 | 2.39E−97 |
| 37 | 4.65E−43 | 6.88E−21 | 4.22E−113 | 2.39E−97 |
| 38 | 1.60E−42 | 7.01E−24 | 4.84E−112 | 7.55E−91 |
| 39 | 1.54E−44 | 3.19E−20 | 4.64E−110 | 2.85E−100 |
| 40 | 4.20E−36 | 3.14E−19 | 5.76E−108 | 5.09E−88 |
| 41 | 1.65E−39 | 8.87E−19 | 2.41E−107 | 6.05E−97 |
| 42 | 8.79E−40 | 1.09E−14 | 3.00E−106 | 3.22E−94 |
| 43 | 1.21E−43 | 1.71E−19 | 1.58E−102 | 1.41E−92 |
| 44 | 2.98E−44 | 8.40E−19 | 1.07E−101 | 4.03E−92 |
| 45 | 8.13E−44 | 8.40E−19 | 1.07E−101 | 4.03E−92 |
| 46 | 2.94E−31 | 2.14E−17 | 5.26E−101 | 5.94E−79 |
| 47 | 1.67E−43 | 1.76E−21 | 8.02E−101 | 1.17E−94 |
| 48 | 2.19E−35 | 1.61E−13 | 1.79E−95 | 4.56E−88 |
| 49 | 4.69E−30 | 2.14E−17 | 1.80E−94 | 5.69E−74 |
| 50 | 3.32E−41 | 8.45E−15 | 1.32E−71 | 2.83E−94 |

TABLE 23h
Data for Staphylococcus, particularly S. aureus (continued)

| | Penicillin | | | |
|---|---|---|---|---|
| No. | G_pv_adj | Piperacillin/Tazobactam_pv_adj | Tetracyclin_pv_adj | Tobramycin_pv_adj |
| 1 | 1.37E−41 | 1.55E−21 | 1.22E−01 | 2.16E−15 |
| 2 | 1.37E−41 | 1.55E−21 | 1.22E−01 | 2.16E−15 |
| 3 | 1.51E−41 | 1.55E−21 | 9.63E−02 | 2.16E−15 |
| 4 | 3.46E−40 | 1.17E−19 | 2.75E−02 | 6.21E−16 |
| 5 | 6.83E−41 | 2.25E−18 | 3.70E−02 | 2.16E−15 |
| 6 | 3.80E−41 | 1.59E−17 | 1.27E−02 | 1.94E−13 |
| 7 | 6.95E−35 | 3.69E−14 | 5.90E−03 | 7.81E−15 |
| 8 | 6.95E−35 | 3.69E−14 | 5.90E−03 | 7.81E−15 |
| 9 | 6.95E−35 | 3.69E−14 | 5.90E−03 | 7.81E−15 |
| 10 | 1.50E−34 | 3.69E−14 | 8.03E−03 | 7.81E−15 |
| 11 | 1.50E−34 | 3.69E−14 | 8.03E−03 | 7.81E−15 |
| 12 | 5.97E−34 | 3.69E−14 | 8.79E−03 | 7.81E−15 |
| 13 | 5.30E−34 | 5.46E−13 | 9.97E−04 | 9.71E−16 |
| 14 | 1.23E−34 | 3.69E−14 | 1.42E−03 | 7.81E−15 |
| 15 | 1.23E−34 | 3.69E−14 | 1.42E−03 | 7.81E−15 |
| 16 | 1.23E−34 | 3.69E−14 | 1.42E−03 | 7.81E−15 |
| 17 | 1.23E−34 | 3.69E−14 | 1.42E−03 | 7.81E−15 |
| 18 | 1.23E−34 | 5.46E−13 | 1.42E−03 | 3.02E−14 |
| 19 | 1.23E−34 | 5.46E−13 | 1.42E−03 | 3.02E−14 |
| 20 | 5.10E−21 | 3.04E−11 | 5.98E−04 | 7.15E−15 |
| 21 | 9.90E−21 | 3.04E−11 | 2.52E−04 | 7.15E−15 |
| 22 | 2.82E−20 | 9.29E−11 | 5.97E−04 | 2.24E−14 |
| 23 | 2.82E−20 | 9.29E−11 | 5.97E−04 | 2.24E−14 |
| 24 | 1.92E−20 | 4.28E−09 | 8.65E−04 | 4.98E−13 |
| 25 | 1.42E−21 | 6.78E−10 | 2.40E−03 | 6.71E−15 |
| 26 | 2.07E−10 | 4.04E−10 | 5.35E−05 | 4.17E−12 |
| 27 | 1.36E−10 | 1.16E−09 | 2.27E−05 | 1.33E−11 |
| 28 | 2.18E−10 | 1.16E−09 | 3.46E−05 | 1.33E−11 |
| 29 | 4.59E−14 | 1.16E−09 | 8.29E−05 | 7.56E−11 |
| 30 | 6.54E−10 | 2.97E−08 | 3.46E−05 | 2.92E−10 |
| 31 | 4.06E−09 | 7.73E−08 | 2.02E−05 | 2.25E−08 |
| 32 | 2.64E−11 | 3.31E−11 | 2.07E−02 | 6.23E−11 |
| 33 | 2.28E−19 | 2.60E−08 | 2.52E−06 | 9.88E−10 |
| 34 | 1.20E−07 | 3.78E−10 | 1.24E−02 | 9.10E−11 |
| 35 | 4.14E−07 | 1.59E−06 | 1.78E−06 | 6.53E−07 |
| 36 | 4.05E−13 | 7.74E−09 | 1.25E−04 | 1.01E−10 |
| 37 | 4.05E−13 | 7.74E−09 | 1.25E−04 | 1.01E−10 |
| 38 | 4.22E−06 | 1.49E−07 | 1.46E−05 | 5.29E−08 |
| 39 | 1.18E−06 | 8.70E−08 | 2.19E−04 | 7.09E−11 |
| 40 | 1.87E−06 | 1.59E−06 | 3.63E−05 | 1.92E−07 |
| 41 | 4.31E−06 | 6.90E−07 | 1.08E−04 | 2.74E−09 |
| 42 | 6.34E−11 | 4.80E−05 | 2.10E−05 | 6.87E−09 |
| 43 | 2.66E−09 | 4.09E−08 | 4.92E−04 | 1.94E−10 |
| 44 | 7.22E−09 | 8.70E−08 | 3.32E−04 | 6.07E−10 |
| 45 | 2.79E−09 | 8.70E−08 | 3.32E−04 | 6.07E−10 |
| 46 | 1.54E−26 | 8.34E−08 | 2.05E−05 | 1.24E−11 |
| 47 | 4.32E−03 | 1.16E−09 | 4.05E−06 | 1.33E−11 |
| 48 | 1.28E−06 | 1.86E−05 | 1.61E−03 | 1.89E−08 |
| 49 | 3.43E−27 | 8.34E−08 | 1.11E−03 | 1.24E−11 |
| 50 | 2.12E−15 | 1.05E−10 | 6.95E−03 | 1.62E−07 |

TABLE 23i
Data for Staphylococcus, particularly S. aureus (continued)

| No. | Locus.Tag | Gene.Symbol | GenBank.Accession | Chromosome |
|---|---|---|---|---|
| 1 | HMPREF0791_1752 | mecA | ZP_04797671 | |
| 2 | HMPREF0794_1488 | ugpQ | ZP_06614206 | |
| 3 | HMPREF0794_1485 | mecR | ZP_06614203 | |
| 4 | SIAG_00762 | | ZP_06819251 | |
| 5 | HMPREF0789_1682 | | ZP_04825874 | |
| 6 | SAMG_01950 | | ZP_05682863 | |
| 7 | Missing locus_tag: FN433596.cds78.83191..82796 | | CBI47961 | |
| 8 | SATW20_00761 | | CBI67631 | |
| 9 | SERP2515 | | YP_190057 | |
| 10 | SAIG_02579 | | ZP_05694555 | |
| 11 | SERP2519 | mecI | YP_190060 | |
| 12 | SERP2505 | | YP_190047 | |
| 13 | HMPREF0794_1451 | | ZP_06614169 | |
| 14 | SERP2490 | kdeP | YP_190033 | |
| 15 | SERP2487 | kdpA | YP_190030 | |
| 16 | SERP2485 | kdpC | YP_190028 | |
| 17 | SERP2489 | kdpD | YP_190032 | |
| 18 | SERP2486 | kdpB | YP_190029 | |
| 19 | SAIG_01884 | | ZP_05693385 | |
| 20 | SERP1347 | tnpA-2 | YP_188919 | |
| 21 | SERP1223 | tnpB-1 | YP_188797 | |
| 22 | SLAG_02844 | | ZP_06930609 | |
| 23 | SA0049 | ant(9) | NP_373289 | |
| 24 | SERP1342 | | YP_188914 | |
| 25 | HMPREF0773_2426 | radC | ZP_04829125 | |
| 26 | SAFG_01021 | | ZP_05703394 | |
| 27 | SAFG_01015 | | ZP_05703388 | |
| 28 | SA2981_2149 | | ADC38360 | |
| 29 | ECTR2_1640 | | CBX35020 | |
| 30 | SA2981_1770 | | ADC37981 | |
| 31 | SAB0169 | | YP_415679 | |
| 32 | SaurJH1_0467 | | YP_001315613 | |
| 33 | SERP1345 | tnpC-2 | YP_188917 | |
| 34 | SGAG_02626 | | ZP_06303506 | |
| 35 | ECTR2_2075 | | CBX35404 | |
| 36 | HMPREF0774_2643 | | ZP_04869332 | |
| 37 | SAHG_01730 | | ZP_05696261 | |
| 38 | SAA6159_00204 | coa | ADL22160 | |
| 39 | SARG_01832 | | ZP_06332611 | |
| 40 | SAIG_01373 | | ZP_05693621 | |
| 41 | SANG_00111 | | ZP_05682471 | |
| 42 | SAHG_02693 | | ZP_05696106 | |
| 43 | SAJG_00740 | | ZP_05692517 | |
| 44 | SaurJH1_0188 | | YP_001315337 | |
| 45 | SGAG_00714 | | ZP_06301594 | |
| 46 | ECTR2_2580 | | CBX35910 | plasmid |
| | | | | pLUH02 |
| 47 | SA2981_0414 | lpl3 | ADC36625 | |
| 48 | SA2981_1977 | agrB | ADC38188 | |
| 49 | SMAG_02765 | | ZP_06817387 | |
| 50 | HMPREF0798_02109 | | ZP_07844597 | |

TABLE 23j
Data for Staphylococcus, particularly S. aureus (continued)

| No. | Start.Coord | End.Coord | Strand | Scaffold.ID |
|---|---|---|---|---|
| 1 | 1335 | 3341 | + | 645076738 |
| 2 | 7348 | 8091 | − | 647019109 |
| 3 | 3685 | 4599 | − | 647019109 |
| 4 | 3987 | 5336 | − | 647537018 |
| 5 | 1842 | 3491 | − | 645076234 |
| 6 | 89170 | 89277 | − | 645958292 |
| 7 | 82796 | 84130 | − | 646862418 |
| 8 | 81146 | 81214 | − | 646862418 |
| 9 | 2570266 | 2571330 | + | 637000098 |
| 10 | 6265 | 7374 | + | 645958144 |
| 11 | 2574428 | 2574799 | − | 637000098 |
| 12 | 2562107 | 2562424 | + | 637000098 |
| 13 | 8938 | 9252 | + | 647019108 |
| 14 | 2549489 | 2550184 | + | 637000098 |
| 15 | 2544901 | 2546577 | − | 637000098 |
| 16 | 2542288 | 2542845 | − | 637000098 |
| 17 | 2546845 | 2549514 | + | 637000098 |
| 18 | 2542861 | 2544882 | − | 637000098 |
| 19 | 76580 | 76657 | − | 645958116 |
| 20 | 1403752 | 1404837 | − | 637000098 |
| 21 | 1255425 | 1257317 | − | 637000098 |
| 22 | 4557 | 5288 | − | 647016972 |
| 23 | 56859 | 57641 | − | 637000072 |
| 24 | 1399547 | 1399606 | + | 637000098 |
| 25 | 4478 | 5164 | − | 645076413 |
| 26 | 277066 | 278082 | + | 645957989 |
| 27 | 272112 | 272231 | − | 645957989 |
| 28 | 2299803 | 2300126 | − | 646862390 |
| 29 | 1804139 | 1805368 | − | 651053129 |
| 30 | 1899111 | 1899692 | + | 646862390 |
| 31 | 209129 | 211012 | + | 637000460 |
| 32 | 487880 | 489091 | + | 640753011 |
| 33 | 1401479 | 1401856 | − | 637000098 |
| 34 | 245 | 686 | − | 647008393 |
| 35 | 2210164 | 2210319 | − | 651053129 |
| 36 | 16441 | 17124 | + | 645076159 |
| 37 | 85620 | 86303 | + | 645958072 |
| 38 | 242193 | 244169 | + | 648231718 |
| 39 | 177946 | 180372 | + | 647535981 |
| 40 | 38140 | 39216 | + | 645958123 |
| 41 | 77988 | 79490 | − | 645958383 |
| 42 | 5009 | 5929 | − | 645958068 |
| 43 | 78131 | 78853 | + | 645958189 |
| 44 | 221049 | 221729 | + | 640753011 |
| 45 | 44419 | 44616 | + | 647008364 |
| 46 | 12682 | 13458 | − | 651053131 |
| 47 | 456367 | 457155 | + | 646862390 |
| 48 | 2096829 | 2097392 | + | 646862390 |
| 49 | 14918 | 15697 | − | 647017059 |
| 50 | 54037 | 54601 | + | 651285048 |

TABLE 24a
Data for Staphylococcus, particularly S. aureus

| No. | Scaffold.External.Accession | Scaffold.Name | best_pv |
|---|---|---|---|
| 1 | NZ_ACJC01000132 | Staphylococcus epidermidis W23144: NZ_ACJC01000132 | 9.38479881072327e−227 |
| 2 | NZ_ADMU01000036 | Staphylococcus epidermidis M23864: W2 (grey) contig00037: | 9.38479881072327e−227 |
| | | NZ_ADMU01000036 | |
| 3 | NZ_ADMU01000036 | Staphylococcus epidermidis M23864: W2 (grey) contig00037: | 2.67435403500704e−225 |
| | | NZ_ADMU01000036 | |
| 4 | NZ_GG770515 | Staphylococcus aureus subsp. aureus EMRSA16 genomic scaffold | 1.77186061517335e−211 |
| | | supercont1.3: NZ_GG770515 | |
| 5 | NZ_ACHE01000064 | Staphylococcus epidermidis BCM-HMP0060: NZ_ACHE01000064 | 3.22333061264623e−210 |
| 6 | NZ_ACKJ01000008 | Staphylococcus aureus A9719: NZ_ACKJ01000008 | 4.28801157250818e−188 |
| 7 | FN433596 | Staphylococcus aureus subsp. aureus TW20: FN433596 | 6.72085159673615e−181 |
| 8 | FN433596 | Staphylococcus aureus subsp. aureus TW20: FN433596 | 6.72085159673615e−181 |
| 9 | NC_002976 | Staphylococcus epidermidis RP62A: NC_002976 | 6.72085159673615e−181 |
| 10 | NZ_ACKF01000035 | Staphylococcus aureus A6300: NZ_ACKF01000035 | 8.5426103876016e−179 |
| 11 | NC_002976 | Staphylococcus epidermidis RP62A: NC_002976 | 8.5426103876016e−179 |
| 12 | NC_002976 | Staphylococcus epidermidis RP62A: NC_002976 | 1.81714784068785e−175 |
| 13 | NZ_ADMU01000035 | Staphylococcus epidermidis M23864: W2 (grey) contig00036: | 3.23410033711171e−175 |
| | | NZ_ADMU01000035 | |
| 14 | NC_002976 | Staphylococcus epidermidis RP62A: NC_002976 | 9.12700235375297e−175 |
| 15 | NC_002976 | Staphylococcus epidermidis RP62A: NC_002976 | 9.12700235375297e−175 |
| 16 | NC_002976 | Staphylococcus epidermidis RP62A: NC_002976 | 9.12700235375297e−175 |
| 17 | NC_002976 | Staphylococcus epidermidis RP62A: NC_002976 | 9.12700235375297e−175 |
| 18 | NC_002976 | Staphylococcus epidermidis RP62A: NC_002976 | 2.25944344923286e−173 |
| 19 | NZ_ACKF01000007 | Staphylococcus aureus A6300: NZ_ACKF01000007 | 2.40063417489507e−172 |
| 20 | NC_002976 | Staphylococcus epidermidis RP62A: NC_002976 | 2.59802993040457e−167 |
| 21 | NC_002976 | Staphylococcus epidermidis RP62A: NC_002976 | 3.12953724144125e−166 |
| 22 | NZ_ADJJ01000031 | Staphylococcus aureus A8796 cont1.31: NZ_ADJJ01000031 | 3.37208086862656e−166 |
| 23 | NC_002745 | Staphylococcus aureus subsp. aureus N315: NC_002745 | 3.37208086862656e−166 |
| 24 | NC_002976 | Staphylococcus epidermidis RP62A: NC_002976 | 7.17319474297637e−161 |
| 25 | NZ_ACHH01000128 | Staphylococcus aureus subsp. aureus TCH70: NZ_ACHH01000128 | 4.16856451727179e−152 |
| 26 | NZ_ACKC01000026 | Staphylococcus aureus A5937: NZ_ACKC01000026 | 1.58751373713434e−135 |
| 27 | NZ_ACKC01000026 | Staphylococcus aureus A5937: NZ_ACKC01000026 | 1.69633422486642e−135 |
| 28 | CP001844 | Staphylococcus aureus 04-02981: CP001844 | 1.77656315889944e−134 |
| 29 | FR714927 | Staphylococcus aureus subsp. aureus ECT-R 2: FR714927 | 4.06264970457926e−133 |
| 30 | CP001844 | Staphylococcus aureus 04-02981: CP001844 | 1.01251954267327e−130 |
| 31 | NC_007622 | Staphylococcus aureus RF122: NC_007622 | 4.84169534955158e−126 |
| 32 | NC_009632 | Staphylococcus aureus subsp. aureus JH1: NC_009632 | 6.20485118760467e−122 |
| 33 | NC_002976 | Staphylococcus epidermidis RP62A: NC_002976 | 5.75915090813908e−120 |
| 34 | NZ_ACYO01000032 | Staphylococcus aureus A8117 cont1.32: NZ_ACYO01000032 | 1.84672228386398e−116 |
| 35 | FR714927 | Staphylococcus aureus subsp. aureus ECT-R 2: FR714927 | 7.1946440374743e−116 |
| 36 | NZ_ACHD01000273 | Staphylococcus aureus subsp. aureus TCH130: NZ_ACHD01000273 | 4.22320321058421e−113 |
| 37 | NZ_ACKE01000012 | Staphylococcus aureus A6224: NZ_ACKE01000012 | 4.22320321058421e−113 |
| 38 | CP002114 | Staphylococcus aureus subsp. aureus JKD6159: CP002114 | 4.83609713640718e−112 |
| 39 | NZ_GG730131 | Staphylococcus aureus subsp. aureus C101 genomic scaffold | 4.63942446669448e−110 |
| | | supercont1.12: NZ_GG730131 | |
| 40 | NZ_ACKF01000014 | Staphylococcus aureus A6300: NZ_ACKF01000014 | 5.76090691723747e−108 |
| 41 | NZ_ACKK01000039 | Staphylococcus aureus A9763: NZ_ACKK01000039 | 2.40583396327414e−107 |
| 42 | NZ_ACKE01000008 | Staphylococcus aureus A6224: NZ_ACKE01000008 | 2.99974766558167e−106 |
| 43 | NZ_ACKG01000031 | Staphylococcus aureus A8115: NZ_ACKG01000031 | 1.57559858677166e−102 |
| 44 | NC_009632 | Staphylococcus aureus subsp. aureus JH1: NC_009632 | 1.0725409335823e−101 |
| 45 | NZ_ACYO01000003 | Staphylococcus aureus A8117 cont1.3: NZ_ACYO01000003 | 1.0725409335823e−101 |
| 46 | FR714929 | Staphylococcus aureus subsp. aureus ECT-R 2 plasmid pLUH02: | 5.26123840198017e−101 |
| | | FR714929 | |
| 47 | CP001844 | Staphylococcus aureus 04-02981: CP001844 | 8.01600909282956e−101 |
| 48 | CP001844 | Staphylococcus aureus 04-02981: CP001844 | 1.78546333248495e−95 |
| 49 | NZ_ADJK01000020 | Staphylococcus aureus A8819 cont1.20: NZ_ADJK01000020 | 1.80469278143707e−94 |
| 50 | NZ_GL545262 | Staphylococcus hominis subsp. hominis C80 genomic scaffold | 2.82728623419544e−94 |
| | | supercont1.11: NZ_GL545262 | |

TABLE 24b
Data for Staphylococcus, particularly S. aureus (continued)

| No. | sign_phenos |
|---|---|
| 1 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 2 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 3 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 4 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 5 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 6 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tobramycin |
| 7 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 8 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 9 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 10 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 11 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 12 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 13 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 14 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 15 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 16 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 17 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 18 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 19 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 20 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 21 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 22 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 23 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 24 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 25 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 26 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 27 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 28 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 29 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 30 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 31 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 32 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tobramycin |
| 33 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 34 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tobramycin |
| 35 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 36 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 37 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 38 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 39 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 40 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 41 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 42 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 43 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 44 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 45 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 46 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 47 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 48 | Ampicillin/Sulbactam; Cefepim; Cefotaxim; Cefuroxim; Ciprofloxacin; Clindamycin; Erythromycin; Imipenem; Levofloxacin; |
| | Moxifloxacin; Oxacillin; Penicillin G; Piperacillin/Tazobactam; Tetracyclin; Tobramycin |
| 49 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |
| 50 | Ampicillin; Ampicillin/Sulbactam; Cefepim; Cefotaxim; Cefuroxim; Ciprofloxacin; Clindamycin; Erythromycin; Imipenem; |
| | Levofloxacin; Moxifloxacin; Oxacillin; Penicillin G; Piperacillin/Tazobactam; Tetracyclin; Tobramycin |

TABLE 24c
Data for Staphylococcus, particularly S. aureus (continued)

| No. | sign_phenos_class | best_pheno | best_pheno_class |
|---|---|---|---|
| 1 | aminoglycoside; fluoroquinolone; lactam; lincosamide; macrolide | Oxacillin | lactam |
| 2 | aminoglycoside; fluoroquinolone; lactam; lincosamide; macrolide | Oxacillin | lactam |
| 3 | aminoglycoside; fluoroquinolone; lactam; lincosamide; macrolide | Oxacillin | lactam |
| 4 | aminoglycoside; fluoroquinolone; lactam; lincosamide; macrolide | Oxacillin | lactam |
| 5 | aminoglycoside; fluoroquinolone; lactam; lincosamide; macrolide | Oxacillin | lactam |
| 6 | aminoglycoside; fluoroquinolone; lactam; lincosamide; macrolide | Moxifloxacin | fluoroquinolone |
| 7 | aminoglycoside; fluoroquinolone; lactam; lincosamide; macrolide; tetracycline | Oxacillin | lactam |
| 8 | aminoglycoside; fluoroquinolone; lactam; lincosamide; macrolide; tetracycline | Oxacillin | lactam |
| 9 | aminoglycoside; fluoroquinolone; lactam; lincosamide; macrolide; tetracycline | Oxacillin | lactam |
| 10 | aminoglycoside; fluoroquinolone; lactam; lincosamide; macrolide; tetracycline | Oxacillin | lactam |
| 11 | aminoglycoside; fluoroquinolone; lactam; lincosamide; macrolide; tetracycline | Oxacillin | lactam |
| 12 | aminoglycoside; fluoroquinolone; lactam; lincosamide; macrolide; tetracycline | Oxacillin | lactam |
| 13 | aminoglycoside; fluoroquinolone; lactam; lincosamide; macrolide; tetracycline | Oxacillin | lactam |
| 14 | aminoglycoside; fluoroquinolone; lactam; lincosamide; macrolide; tetracycline | Oxacillin | lactam |
| 15 | aminoglycoside; fluoroquinolone; lactam; lincosamide; macrolide; tetracycline | Oxacillin | lactam |
| 16 | aminoglycoside; fluoroquinolone; lactam; lincosamide; macrolide; tetracycline | Oxacillin | lactam |
| 17 | aminoglycoside; fluoroquinolone; lactam; lincosamide; macrolide; tetracycline | Oxacillin | lactam |
| 18 | aminoglycoside; fluoroquinolone; lactam; lincosamide; macrolide; tetracycline | Oxacillin | lactam |
| 19 | aminoglycoside; fluoroquinolone; lactam; lincosamide; macrolide; tetracycline | Oxacillin | lactam |
| 20 | aminoglycoside; fluoroquinolone; lactam; lincosamide; macrolide; tetracycline | Moxifloxacin | fluoroquinolone |
| 21 | aminoglycoside; fluoroquinolone; lactam; lincosamide; macrolide; tetracycline | Moxifloxacin | fluoroquinolone |
| 22 | aminoglycoside; fluoroquinolone; lactam; lincosamide; macrolide; tetracycline | Moxifloxacin | fluoroquinolone |
| 23 | aminoglycoside; fluoroquinolone; lactam; lincosamide; macrolide; tetracycline | Moxifloxacin | fluoroquinolone |
| 24 | aminoglycoside; fluoroquinolone; lactam; lincosamide; macrolide; tetracycline | Moxifloxacin | fluoroquinolone |
| 25 | aminoglycoside; fluoroquinolone; lactam; lincosamide; macrolide; tetracycline | Moxifloxacin | fluoroquinolone |
| 26 | aminoglycoside; fluoroquinolone; lactam; lincosamide; macrolide; tetracycline | Moxifloxacin | fluoroquinolone |
| 27 | aminoglycoside; fluoroquinolone; lactam; lincosamide; macrolide; tetracycline | Moxifloxacin | fluoroquinolone |
| 28 | aminoglycoside; fluoroquinolone; lactam; lincosamide; macrolide; tetracycline | Moxifloxacin | fluoroquinolone |
| 29 | aminoglycoside; fluoroquinolone; lactam; lincosamide; macrolide; tetracycline | Moxifloxacin | fluoroquinolone |
| 30 | aminoglycoside; fluoroquinolone; lactam; lincosamide; macrolide; tetracycline | Moxifloxacin | fluoroquinolone |
| 31 | aminoglycoside; fluoroquinolone; lactam; lincosamide; macrolide; tetracycline | Moxifloxacin | fluoroquinolone |
| 32 | aminoglycoside; fluoroquinolone; lactam; lincosamide; macrolide | Moxifloxacin | fluoroquinolone |
| 33 | aminoglycoside; fluoroquinolone; lactam; lincosamide; macrolide; tetracycline | Moxifloxacin | fluoroquinolone |
| 34 | aminoglycoside; fluoroquinolone; lactam; lincosamide; macrolide | Moxifloxacin | fluoroquinolone |
| 35 | aminoglycoside; fluoroquinolone; lactam; lincosamide; macrolide; tetracycline | Moxifloxacin | fluoroquinolone |
| 36 | aminoglycoside; fluoroquinolone; lactam; lincosamide; macrolide; tetracycline | Moxifloxacin | fluoroquinolone |
| 37 | aminoglycoside; fluoroquinolone; lactam; lincosamide; macrolide; tetracycline | Moxifloxacin | fluoroquinolone |
| 38 | aminoglycoside; fluoroquinolone; lactam; lincosamide; macrolide; tetracycline | Moxifloxacin | fluoroquinolone |
| 39 | aminoglycoside; fluoroquinolone; lactam; lincosamide; macrolide; tetracycline | Moxifloxacin | fluoroquinolone |
| 40 | aminoglycoside; fluoroquinolone; lactam; lincosamide; macrolide; tetracycline | Moxifloxacin | fluoroquinolone |
| 41 | aminoglycoside; fluoroquinolone; lactam; lincosamide; macrolide; tetracycline | Moxifloxacin | fluoroquinolone |
| 42 | aminoglycoside; fluoroquinolone; lactam; lincosamide; macrolide; tetracycline | Moxifloxacin | fluoroquinolone |
| 43 | aminoglycoside; fluoroquinolone; lactam; lincosamide; macrolide; tetracycline | Moxifloxacin | fluoroquinolone |
| 44 | aminoglycoside; fluoroquinolone; lactam; lincosamide; macrolide; tetracycline | Moxifloxacin | fluoroquinolone |
| 45 | aminoglycoside; fluoroquinolone; lactam; lincosamide; macrolide; tetracycline | Moxifloxacin | fluoroquinolone |
| 46 | aminoglycoside; fluoroquinolone; lactam; lincosamide; macrolide; tetracycline | Moxifloxacin | fluoroquinolone |
| 47 | aminoglycoside; fluoroquinolone; lactam; lincosamide; macrolide; tetracycline | Moxifloxacin | fluoroquinolone |
| 48 | aminoglycoside; fluoroquinolone; lactam; lincosamide; macrolide; tetracycline | Moxifloxacin | fluoroquinolone |
| 49 | aminoglycoside; fluoroquinolone; lactam; lincosamide; macrolide; tetracycline | Moxifloxacin | fluoroquinolone |
| 50 | aminoglycoside; fluoroquinolone; lactam; lincosamide; macrolide; tetracycline | Oxacillin | lactam |

TABLE 24d
Data for Staphylococcus, particularly S. aureus (continued)

| No. | num_aminoglycoside | num_fluoroquinolone | num_lactam | num_lincosamide | num_macrolide | num_tetracycline |
|---|---|---|---|---|---|---|
| 1 | 1 | 3 | 9 | 1 | 1 | 0 |
| 2 | 1 | 3 | 9 | 1 | 1 | 0 |
| 3 | 1 | 3 | 9 | 1 | 1 | 0 |
| 4 | 1 | 3 | 9 | 1 | 1 | 0 |
| 5 | 1 | 3 | 9 | 1 | 1 | 0 |
| 6 | 1 | 3 | 9 | 1 | 1 | 0 |
| 7 | 1 | 3 | 9 | 1 | 1 | 1 |
| 8 | 1 | 3 | 9 | 1 | 1 | 1 |
| 9 | 1 | 3 | 9 | 1 | 1 | 1 |
| 10 | 1 | 3 | 9 | 1 | 1 | 1 |
| 11 | 1 | 3 | 9 | 1 | 1 | 1 |
| 12 | 1 | 3 | 9 | 1 | 1 | 1 |
| 13 | 1 | 3 | 9 | 1 | 1 | 1 |
| 14 | 1 | 3 | 9 | 1 | 1 | 1 |
| 15 | 1 | 3 | 9 | 1 | 1 | 1 |
| 16 | 1 | 3 | 9 | 1 | 1 | 1 |
| 17 | 1 | 3 | 9 | 1 | 1 | 1 |
| 18 | 1 | 3 | 9 | 1 | 1 | 1 |
| 19 | 1 | 3 | 9 | 1 | 1 | 1 |
| 20 | 1 | 3 | 9 | 1 | 1 | 1 |
| 21 | 1 | 3 | 9 | 1 | 1 | 1 |
| 22 | 1 | 3 | 9 | 1 | 1 | 1 |
| 23 | 1 | 3 | 9 | 1 | 1 | 1 |
| 24 | 1 | 3 | 9 | 1 | 1 | 1 |
| 25 | 1 | 3 | 9 | 1 | 1 | 1 |
| 26 | 1 | 3 | 8 | 1 | 1 | 1 |
| 27 | 1 | 3 | 8 | 1 | 1 | 1 |
| 28 | 1 | 3 | 8 | 1 | 1 | 1 |
| 29 | 1 | 3 | 8 | 1 | 1 | 1 |
| 30 | 1 | 3 | 8 | 1 | 1 | 1 |
| 31 | 1 | 3 | 9 | 1 | 1 | 1 |
| 32 | 1 | 3 | 8 | 1 | 1 | 0 |
| 33 | 1 | 3 | 9 | 1 | 1 | 1 |
| 34 | 1 | 3 | 8 | 1 | 1 | 0 |
| 35 | 1 | 3 | 8 | 1 | 1 | 1 |
| 36 | 1 | 3 | 9 | 1 | 1 | 1 |
| 37 | 1 | 3 | 9 | 1 | 1 | 1 |
| 38 | 1 | 3 | 8 | 1 | 1 | 1 |
| 39 | 1 | 3 | 8 | 1 | 1 | 1 |
| 40 | 1 | 3 | 8 | 1 | 1 | 1 |
| 41 | 1 | 3 | 8 | 1 | 1 | 1 |
| 42 | 1 | 3 | 8 | 1 | 1 | 1 |
| 43 | 1 | 3 | 8 | 1 | 1 | 1 |
| 44 | 1 | 3 | 8 | 1 | 1 | 1 |
| 45 | 1 | 3 | 8 | 1 | 1 | 1 |
| 46 | 1 | 3 | 9 | 1 | 1 | 1 |
| 47 | 1 | 3 | 8 | 1 | 1 | 1 |
| 48 | 1 | 3 | 8 | 1 | 1 | 1 |
| 49 | 1 | 3 | 9 | 1 | 1 | 1 |
| 50 | 1 | 3 | 9 | 1 | 1 | 1 |

TABLE 24e
Data for Staphylococcus, particularly S. aureus (continued)

| No. | Ampicillin_pv_adj | Ampicillin/Sulbactam_pv_adj | Cefepim_pv_adj | Cefotaxim_pv_adj |
|---|---|---|---|---|
| 1 | 2.20E−08 | 5.96E−37 | 5.96E−37 | 5.96E−37 |
| 2 | 2.20E−08 | 5.96E−37 | 5.96E−37 | 5.96E−37 |
| 3 | 2.20E−08 | 5.96E−37 | 5.96E−37 | 5.96E−37 |
| 4 | 4.64E−08 | 4.55E−35 | 4.55E−35 | 4.55E−35 |
| 5 | 2.20E−08 | 2.16E−33 | 2.16E−33 | 2.16E−33 |
| 6 | 2.22E−09 | 1.64E−31 | 1.64E−31 | 1.64E−31 |
| 7 | 3.22E−07 | 2.72E−27 | 2.72E−27 | 2.72E−27 |
| 8 | 3.22E−07 | 2.72E−27 | 2.72E−27 | 2.72E−27 |
| 9 | 3.22E−07 | 2.72E−27 | 2.72E−27 | 2.72E−27 |
| 10 | 3.22E−07 | 2.72E−27 | 2.72E−27 | 2.72E−27 |
| 11 | 3.22E−07 | 2.72E−27 | 2.72E−27 | 2.72E−27 |
| 12 | 6.85E−07 | 2.72E−27 | 2.72E−27 | 2.72E−27 |
| 13 | 2.90E−07 | 1.05E−25 | 1.05E−25 | 1.05E−25 |
| 14 | 3.22E−07 | 2.72E−27 | 2.72E−27 | 2.72E−27 |
| 15 | 3.22E−07 | 2.72E−27 | 2.72E−27 | 2.72E−27 |
| 16 | 3.22E−07 | 2.72E−27 | 2.72E−27 | 2.72E−27 |
| 17 | 3.22E−07 | 2.72E−27 | 2.72E−27 | 2.72E−27 |
| 18 | 2.90E−07 | 1.05E−25 | 1.05E−25 | 1.05E−25 |
| 19 | 2.90E−07 | 1.05E−25 | 1.05E−25 | 1.05E−25 |
| 20 | 8.65E−04 | 1.18E−21 | 1.18E−21 | 1.18E−21 |
| 21 | 8.65E−04 | 1.18E−21 | 1.18E−21 | 1.18E−21 |
| 22 | 3.21E−03 | 7.21E−21 | 7.21E−21 | 7.21E−21 |
| 23 | 3.21E−03 | 7.21E−21 | 7.21E−21 | 7.21E−21 |
| 24 | 1.55E−03 | 1.27E−19 | 1.27E−19 | 1.27E−19 |
| 25 | 1.83E−03 | 2.01E−19 | 2.01E−19 | 2.01E−19 |
| 26 | 1.26E−02 | 1.32E−20 | 1.32E−20 | 1.32E−20 |
| 27 | 1.18E−02 | 7.81E−20 | 7.81E−20 | 7.81E−20 |
| 28 | 1.18E−02 | 7.81E−20 | 7.81E−20 | 7.81E−20 |
| 29 | 1.26E−02 | 7.25E−19 | 7.25E−19 | 7.25E−19 |
| 30 | 3.28E−02 | 3.76E−18 | 3.76E−18 | 3.76E−18 |
| 31 | 4.05E−03 | 1.35E−15 | 1.35E−15 | 1.35E−15 |
| 32 | 1.24E−02 | 1.13E−19 | 1.13E−19 | 1.13E−19 |
| 33 | 1.11E−03 | 1.73E−15 | 1.73E−15 | 1.73E−15 |
| 34 | 1.27E−02 | 6.35E−19 | 6.35E−19 | 6.35E−19 |
| 35 | 2.32E−02 | 3.05E−13 | 3.05E−13 | 3.05E−13 |
| 36 | 7.10E−03 | 2.03E−18 | 2.03E−18 | 2.03E−18 |
| 37 | 7.10E−03 | 2.03E−18 | 2.03E−18 | 2.03E−18 |
| 38 | 1.24E−02 | 2.91E−15 | 2.91E−15 | 2.91E−15 |
| 39 | 3.37E−02 | 1.60E−16 | 1.60E−16 | 1.60E−16 |
| 40 | 5.70E−02 | 1.88E−12 | 1.88E−12 | 1.88E−12 |
| 41 | 1.15E−01 | 1.25E−15 | 1.25E−15 | 1.25E−15 |
| 42 | 3.84E−02 | 9.78E−11 | 9.78E−11 | 9.78E−11 |
| 43 | 3.56E−02 | 5.77E−17 | 5.77E−17 | 5.77E−17 |
| 44 | 3.37E−02 | 1.60E−16 | 1.60E−16 | 1.60E−16 |
| 45 | 3.37E−02 | 1.60E−16 | 1.60E−16 | 1.60E−16 |
| 46 | 2.90E−07 | 1.61E−15 | 1.61E−15 | 1.61E−15 |
| 47 | 7.69E−02 | 7.81E−20 | 7.81E−20 | 7.81E−20 |
| 48 | 8.07E−02 | 6.08E−12 | 6.08E−12 | 6.08E−12 |
| 49 | 2.90E−07 | 1.61E−15 | 1.61E−15 | 1.61E−15 |
| 50 | 3.04E−06 | 1.82E−17 | 1.82E−17 | 1.82E−17 |

TABLE 24f
Data for Staphylococcus, particularly S. aureus (continued)

| No. | Cefuroxim_pv_adj | Ciprofloxacin_pv_adj | Clindamycin_pv_adj | Erythromycin_pv_adj |
|---|---|---|---|---|
| 1 | 1.02E−172 | 4.82E−131 | 5.41E−111 | 1.04E−111 |
| 2 | 1.02E−172 | 4.82E−131 | 5.41E−111 | 1.04E−111 |
| 3 | 1.02E−172 | 3.02E−132 | 6.31E−112 | 7.39E−113 |
| 4 | 1.19E−170 | 1.00E−131 | 5.45E−114 | 1.72E−111 |
| 5 | 9.55E−169 | 1.65E−131 | 8.55E−113 | 3.44E−110 |
| 6 | 3.09E−155 | 2.03E−145 | 7.78E−102 | 4.54E−100 |
| 7 | 2.00E−140 | 4.08E−138 | 6.00E−125 | 2.44E−127 |
| 8 | 2.00E−140 | 5.95E−135 | 7.70E−123 | 2.44E−127 |
| 9 | 2.00E−140 | 4.08E−138 | 6.00E−125 | 2.44E−127 |
| 10 | 2.29E−138 | 2.11E−136 | 1.31E−123 | 4.65E−126 |
| 11 | 2.29E−138 | 3.02E−133 | 1.67E−121 | 4.65E−126 |
| 12 | 1.01E−138 | 1.36E−128 | 4.01E−120 | 1.01E−123 |
| 13 | 2.71E−145 | 1.43E−135 | 2.49E−122 | 2.94E−121 |
| 14 | 5.87E−142 | 6.84E−140 | 7.88E−125 | 3.61E−124 |
| 15 | 5.87E−142 | 6.84E−140 | 7.88E−125 | 3.61E−124 |
| 16 | 5.87E−142 | 6.84E−140 | 7.88E−125 | 3.61E−124 |
| 17 | 5.87E−142 | 6.84E−140 | 7.88E−125 | 3.61E−124 |
| 18 | 3.15E−140 | 6.84E−140 | 1.65E−123 | 1.01E−122 |
| 19 | 3.78E−139 | 5.24E−139 | 7.90E−123 | 4.58E−122 |
| 20 | 3.99E−125 | 5.65E−132 | 3.88E−136 | 8.83E−140 |
| 21 | 5.82E−123 | 4.09E−130 | 1.24E−134 | 2.39E−138 |
| 22 | 4.30E−124 | 5.65E−132 | 7.67E−139 | 1.30E−142 |
| 23 | 4.30E−124 | 5.65E−132 | 7.67E−139 | 1.30E−142 |
| 24 | 8.70E−122 | 4.09E−130 | 4.42E−135 | 5.01E−139 |
| 25 | 1.95E−112 | 3.48E−117 | 1.09E−121 | 8.26E−126 |
| 26 | 9.99E−102 | 2.00E−99 | 7.42E−94 | 1.88E−93 |
| 27 | 9.92E−101 | 1.25E−100 | 4.84E−95 | 2.65E−94 |
| 28 | 1.08E−99 | 6.62E−99 | 5.06E−95 | 1.80E−94 |
| 29 | 4.45E−100 | 1.46E−101 | 3.05E−98 | 1.82E−99 |
| 30 | 3.62E−99 | 6.76E−98 | 4.11E−89 | 2.04E−88 |
| 31 | 5.46E−82 | 8.46E−90 | 4.95E−69 | 2.56E−67 |
| 32 | 3.45E−93 | 1.68E−88 | 1.37E−90 | 2.27E−88 |
| 33 | 7.10E−92 | 7.29E−86 | 6.57E−78 | 1.16E−76 |
| 34 | 3.46E−90 | 5.20E−83 | 1.49E−80 | 4.89E−80 |
| 35 | 9.56E−73 | 1.94E−83 | 1.78E−63 | 2.45E−61 |
| 36 | 1.95E−83 | 3.50E−91 | 5.92E−79 | 2.14E−84 |
| 37 | 1.95E−83 | 3.50E−91 | 5.92E−79 | 2.14E−84 |
| 38 | 7.71E−76 | 4.08E−77 | 6.05E−58 | 6.49E−57 |
| 39 | 3.93E−89 | 4.20E−78 | 5.89E−71 | 3.43E−72 |
| 40 | 3.54E−69 | 3.19E−77 | 4.43E−58 | 3.86E−57 |
| 41 | 8.95E−84 | 1.49E−77 | 1.71E−74 | 1.87E−74 |
| 42 | 3.77E−75 | 8.95E−84 | 6.90E−85 | 9.85E−84 |
| 43 | 6.75E−81 | 1.60E−81 | 9.46E−72 | 9.56E−79 |
| 44 | 3.58E−81 | 2.83E−81 | 9.63E−72 | 1.15E−78 |
| 45 | 4.14E−80 | 2.83E−81 | 1.29E−70 | 1.77E−77 |
| 46 | 7.59E−62 | 5.15E−79 | 7.94E−76 | 5.98E−76 |
| 47 | 6.11E−79 | 1.32E−73 | 5.43E−98 | 2.20E−97 |
| 48 | 1.16E−77 | 7.87E−68 | 1.31E−67 | 3.92E−67 |
| 49 | 5.08E−59 | 3.55E−74 | 3.25E−71 | 5.25E−71 |
| 50 | 4.13E−78 | 4.35E−52 | 2.56E−53 | 1.24E−51 |

TABLE 24g
Data for Staphylococcus, particularly S. aureus (continued)

| No. | Imipenem_pv_adj | Levofloxacin_pv_adj | Moxifloxacin_pv_adj | Oxacillin_pv_adj |
|---|---|---|---|---|
| 1 | 2.22E−97 | 8.36E−31 | 1.85E−167 | 9.38E−227 |
| 2 | 2.22E−97 | 8.36E−31 | 1.85E−167 | 9.38E−227 |
| 3 | 2.22E−97 | 8.36E−31 | 1.24E−168 | 2.67E−225 |
| 4 | 5.09E−91 | 5.82E−30 | 1.90E−168 | 1.77E−211 |
| 5 | 2.50E−88 | 1.50E−28 | 3.37E−166 | 3.22E−210 |
| 6 | 1.02E−75 | 5.49E−34 | 4.29E−188 | 2.96E−187 |
| 7 | 5.60E−79 | 1.02E−27 | 1.24E−168 | 6.72E−181 |
| 8 | 5.60E−79 | 1.02E−27 | 8.38E−166 | 6.72E−181 |
| 9 | 5.60E−79 | 1.02E−27 | 1.24E−168 | 6.72E−181 |
| 10 | 5.60E−79 | 1.02E−27 | 9.67E−167 | 8.54E−179 |
| 11 | 7.66E−78 | 1.02E−27 | 6.64E−164 | 8.54E−179 |
| 12 | 5.60E−79 | 5.41E−27 | 4.22E−160 | 1.82E−175 |
| 13 | 1.11E−77 | 1.82E−28 | 7.77E−170 | 3.23E−175 |
| 14 | 5.16E−79 | 1.02E−27 | 1.02E−172 | 9.13E−175 |
| 15 | 5.16E−79 | 1.02E−27 | 1.02E−172 | 9.13E−175 |
| 16 | 5.16E−79 | 1.02E−27 | 1.02E−172 | 9.13E−175 |
| 17 | 5.16E−79 | 1.02E−27 | 1.02E−172 | 9.13E−175 |
| 18 | 1.11E−77 | 4.43E−26 | 5.10E−171 | 2.26E−173 |
| 19 | 1.11E−77 | 4.43E−26 | 4.94E−170 | 2.40E−172 |
| 20 | 2.49E−65 | 7.88E−28 | 2.60E−167 | 1.43E−155 |
| 21 | 7.09E−65 | 7.88E−28 | 3.13E−166 | 2.33E−153 |
| 22 | 1.38E−64 | 8.83E−27 | 3.37E−166 | 1.17E−154 |
| 23 | 1.38E−64 | 8.83E−27 | 3.37E−166 | 1.17E−154 |
| 24 | 6.20E−63 | 2.26E−24 | 7.17E−161 | 3.62E−151 |
| 25 | 1.34E−61 | 1.22E−26 | 4.17E−152 | 4.81E−142 |
| 26 | 6.28E−51 | 3.77E−23 | 1.59E−135 | 2.07E−120 |
| 27 | 6.51E−51 | 2.53E−22 | 1.70E−135 | 8.40E−122 |
| 28 | 6.51E−51 | 2.53E−22 | 1.78E−134 | 8.52E−120 |
| 29 | 5.53E−51 | 1.45E−21 | 4.06E−133 | 1.84E−119 |
| 30 | 8.78E−49 | 9.16E−21 | 1.01E−130 | 3.53E−117 |
| 31 | 8.13E−44 | 1.25E−24 | 4.84E−126 | 3.22E−102 |
| 32 | 2.98E−44 | 2.87E−19 | 6.20E−122 | 5.33E−111 |
| 33 | 6.56E−47 | 5.69E−23 | 5.76E−120 | 7.13E−108 |
| 34 | 4.99E−46 | 2.00E−18 | 1.85E−116 | 2.91E−105 |
| 35 | 1.79E−38 | 1.08E−20 | 7.19E−116 | 1.17E−92 |
| 36 | 4.65E−43 | 6.88E−21 | 4.22E−113 | 2.39E−97 |
| 37 | 4.65E−43 | 6.88E−21 | 4.22E−113 | 2.39E−97 |
| 38 | 1.60E−42 | 7.01E−24 | 4.84E−112 | 7.55E−91 |
| 39 | 1.54E−44 | 3.19E−20 | 4.64E−110 | 2.85E−100 |
| 40 | 4.20E−36 | 3.14E−19 | 5.76E−108 | 5.09E−88 |
| 41 | 1.65E−39 | 8.87E−19 | 2.41E−107 | 6.05E−97 |
| 42 | 8.79E−40 | 1.09E−14 | 3.00E−106 | 3.22E−94 |
| 43 | 1.21E−43 | 1.71E−19 | 1.58E−102 | 1.41E−92 |
| 44 | 2.98E−44 | 8.40E−19 | 1.07E−101 | 4.03E−92 |
| 45 | 8.13E−44 | 8.40E−19 | 1.07E−101 | 4.03E−92 |
| 46 | 2.94E−31 | 2.14E−17 | 5.26E−101 | 5.94E−79 |
| 47 | 1.67E−43 | 1.76E−21 | 8.02E−101 | 1.17E−94 |
| 48 | 2.19E−35 | 1.61E−13 | 1.79E−95 | 4.56E−88 |
| 49 | 4.69E−30 | 2.14E−17 | 1.80E−94 | 5.69E−74 |
| 50 | 3.32E−41 | 8.45E−15 | 1.32E−71 | 2.83E−94 |

TABLE 24h
Data for Staphylococcus, particularly S. aureus (continued)

| No. | Penicillin G_pv_adj | Piperacillin/Tazobactam_pv_adj | Tetracyclin_pv_adj | Tobramycin_pv_adj |
|---|---|---|---|---|
| 1 | 1.37E−41 | 1.55E−21 | 1.22E−01 | 2.16E−15 |
| 2 | 1.37E−41 | 1.55E−21 | 1.22E−01 | 2.16E−15 |
| 3 | 1.51E−41 | 1.55E−21 | 9.63E−02 | 2.16E−15 |
| 4 | 3.46E−40 | 1.17E−19 | 2.75E−02 | 6.21E−16 |
| 5 | 6.83E−41 | 2.25E−18 | 3.70E−02 | 2.16E−15 |
| 6 | 3.80E−41 | 1.59E−17 | 1.27E−02 | 1.94E−13 |
| 7 | 6.95E−35 | 3.69E−14 | 5.90E−03 | 7.81E−15 |
| 8 | 6.95E−35 | 3.69E−14 | 5.90E−03 | 7.81E−15 |
| 9 | 6.95E−35 | 3.69E−14 | 5.90E−03 | 7.81E−15 |
| 10 | 1.50E−34 | 3.69E−14 | 8.03E−03 | 7.81E−15 |
| 11 | 1.50E−34 | 3.69E−14 | 8.03E−03 | 7.81E−15 |
| 12 | 5.97E−34 | 3.69E−14 | 8.79E−03 | 7.81E−15 |
| 13 | 5.30E−34 | 5.46E−13 | 9.97E−04 | 9.71E−16 |
| 14 | 1.23E−34 | 3.69E−14 | 1.42E−03 | 7.81E−15 |
| 15 | 1.23E−34 | 3.69E−14 | 1.42E−03 | 7.81E−15 |
| 16 | 1.23E−34 | 3.69E−14 | 1.42E−03 | 7.81E−15 |
| 17 | 1.23E−34 | 3.69E−14 | 1.42E−03 | 7.81E−15 |
| 18 | 1.23E−34 | 5.46E−13 | 1.42E−03 | 3.02E−14 |
| 19 | 1.23E−34 | 5.46E−13 | 1.42E−03 | 3.02E−14 |
| 20 | 5.10E−21 | 3.04E−11 | 5.98E−04 | 7.15E−15 |
| 21 | 9.90E−21 | 3.04E−11 | 2.52E−04 | 7.15E−15 |
| 22 | 2.82E−20 | 9.29E−11 | 5.97E−04 | 2.24E−14 |
| 23 | 2.82E−20 | 9.29E−11 | 5.97E−04 | 2.24E−14 |
| 24 | 1.92E−20 | 4.28E−09 | 8.65E−04 | 4.98E−13 |
| 25 | 1.42E−21 | 6.78E−10 | 2.40E−03 | 6.71E−15 |
| 26 | 2.07E−10 | 4.04E−10 | 5.35E−05 | 4.17E−12 |
| 27 | 1.36E−10 | 1.16E−09 | 2.27E−05 | 1.33E−11 |
| 28 | 2.18E−10 | 1.16E−09 | 3.46E−05 | 1.33E−11 |
| 29 | 4.59E−14 | 1.16E−09 | 8.29E−05 | 7.56E−11 |
| 30 | 6.54E−10 | 2.97E−08 | 3.46E−05 | 2.92E−10 |
| 31 | 4.06E−09 | 7.73E−08 | 2.02E−05 | 2.25E−08 |
| 32 | 2.64E−11 | 3.31E−11 | 2.07E−02 | 6.23E−11 |
| 33 | 2.28E−19 | 2.60E−08 | 2.52E−06 | 9.88E−10 |
| 34 | 1.20E−07 | 3.78E−10 | 1.24E−02 | 9.10E−11 |
| 35 | 4.14E−07 | 1.59E−06 | 1.78E−06 | 6.53E−07 |
| 36 | 4.05E−13 | 7.74E−09 | 1.25E−04 | 1.01E−10 |
| 37 | 4.05E−13 | 7.74E−09 | 1.25E−04 | 1.01E−10 |
| 38 | 4.22E−06 | 1.49E−07 | 1.46E−05 | 5.29E−08 |
| 39 | 1.18E−06 | 8.70E−08 | 2.19E−04 | 7.09E−11 |
| 40 | 1.87E−06 | 1.59E−06 | 3.63E−05 | 1.92E−07 |
| 41 | 4.31E−06 | 6.90E−07 | 1.08E−04 | 2.74E−09 |
| 42 | 6.34E−11 | 4.80E−05 | 2.10E−05 | 6.87E−09 |
| 43 | 2.66E−09 | 4.09E−08 | 4.92E−04 | 1.94E−10 |
| 44 | 7.22E−09 | 8.70E−08 | 3.32E−04 | 6.07E−10 |
| 45 | 2.79E−09 | 8.70E−08 | 3.32E−04 | 6.07E−10 |
| 46 | 1.54E−26 | 8.34E−08 | 2.05E−05 | 1.24E−11 |
| 47 | 4.32E−03 | 1.16E−09 | 4.05E−06 | 1.33E−11 |
| 48 | 1.28E−06 | 1.86E−05 | 1.61E−03 | 1.89E−08 |
| 49 | 3.43E−27 | 8.34E−08 | 1.11E−03 | 1.24E−11 |
| 50 | 2.12E−15 | 1.05E−10 | 6.95E−03 | 1.62E−07 |

TABLE 24i
Data for Staphylococcus, particularly S. aureus (continued)

| No. | Locus.Tag | Gene.Symbol | GenBank.Accession | Chromosome |
|---|---|---|---|---|
| 1 | HMPREF0791_1752 | mecA | ZP_04797671 | |
| 2 | HMPREF0794_1488 | ugpQ | ZP_06614206 | |
| 3 | HMPREF0794_1485 | mecR | ZP_06614203 | |
| 4 | SIAG_00762 | | ZP_06819251 | |
| 5 | HMPREF0789_1682 | | ZP_04825874 | |
| 6 | SAMG_01950 | | ZP_05682863 | |
| 7 | Missing locus_tag: FN433596.cds78.83191..82796 | | CBI47961 | |
| 8 | SATW20_00761 | | CBI67631 | |
| 9 | SERP2515 | | YP_190057 | |
| 10 | SAIG_02579 | | ZP_05694555 | |
| 11 | SERP2519 | mecI | YP_190060 | |
| 12 | SERP2505 | | YP_190047 | |
| 13 | HMPREF0794_1451 | | ZP_06614169 | |
| 14 | SERP2490 | kdeP | YP_190033 | |
| 15 | SERP2487 | kdpA | YP_190030 | |
| 16 | SERP2485 | kdpC | YP_190028 | |
| 17 | SERP2489 | kdpD | YP_190032 | |
| 18 | SERP2486 | kdpB | YP_190029 | |
| 19 | SAIG_01884 | | ZP_05693385 | |
| 20 | SERP1347 | tnpA-2 | YP_188919 | |
| 21 | SERP1223 | tnpB-1 | YP_188797 | |
| 22 | SLAG_02844 | | ZP_06930609 | |
| 23 | SA0049 | ant(9) | NP_373289 | |
| 24 | SERP1342 | | YP_188914 | |
| 25 | HMPREF0773_2426 | radC | ZP_04829125 | |
| 26 | SAFG_01021 | | ZP_05703394 | |
| 27 | SAFG_01015 | | ZP_05703388 | |
| 28 | SA2981_2149 | | ADC38360 | |
| 29 | ECTR2_1640 | | CBX35020 | |
| 30 | SA2981_1770 | | ADC37981 | |
| 31 | SAB0169 | | YP_415679 | |
| 32 | SaurJH1_0467 | | YP_001315613 | |
| 33 | SERP1345 | tnpC-2 | YP_188917 | |
| 34 | SGAG_02626 | | ZP_06303506 | |
| 35 | ECTR2_2075 | | CBX35404 | |
| 36 | HMPREF0774_2643 | | ZP_04869332 | |
| 37 | SAHG_01730 | | ZP_05696261 | |
| 38 | SAA6159_00204 | coa | ADL22160 | |
| 39 | SARG_01832 | | ZP_06332611 | |
| 40 | SAIG_01373 | | ZP_05693621 | |
| 41 | SANG_00111 | | ZP_05682471 | |
| 42 | SAHG_02693 | | ZP_05696106 | |
| 43 | SAJG_00740 | | ZP_05692517 | |
| 44 | SaurJH1_0188 | | YP_001315337 | |
| 45 | SGAG_00714 | | ZP_06301594 | |
| 46 | ECTR2_2580 | | CBX35910 | plasmid |
| | | | | pLUH02 |
| 47 | SA2981_0414 | lpl3 | ADC36625 | |
| 48 | SA2981_1977 | agrB | ADC38188 | |
| 49 | SMAG_02765 | | ZP_06817387 | |
| 50 | HMPREF0798_02109 | | ZP_07844597 | |

TABLE 24j
Data for Staphylococcus, particularly S. aureus (continued)

| No. | Start.Coord | End.Coord | Strand | Scaffold.ID |
|---|---|---|---|---|
| 1 | 1335 | 3341 | + | 645076738 |
| 2 | 7348 | 8091 | − | 647019109 |
| 3 | 3685 | 4599 | − | 647019109 |
| 4 | 3987 | 5336 | − | 647537018 |
| 5 | 1842 | 3491 | − | 645076234 |
| 6 | 89170 | 89277 | − | 645958292 |
| 7 | 82796 | 84130 | − | 646862418 |
| 8 | 81146 | 81214 | − | 646862418 |
| 9 | 2570266 | 2571330 | + | 637000098 |
| 10 | 6265 | 7374 | + | 645958144 |
| 11 | 2574428 | 2574799 | − | 637000098 |
| 12 | 2562107 | 2562424 | + | 637000098 |
| 13 | 8938 | 9252 | + | 647019108 |
| 14 | 2549489 | 2550184 | + | 637000098 |
| 15 | 2544901 | 2546577 | − | 637000098 |
| 16 | 2542288 | 2542845 | − | 637000098 |
| 17 | 2546845 | 2549514 | + | 637000098 |
| 18 | 2542861 | 2544882 | − | 637000098 |
| 19 | 76580 | 76657 | − | 645958116 |
| 20 | 1403752 | 1404837 | − | 637000098 |
| 21 | 1255425 | 1257317 | − | 637000098 |
| 22 | 4557 | 5288 | − | 647016972 |
| 23 | 56859 | 57641 | − | 637000072 |
| 24 | 1399547 | 1399606 | + | 637000098 |
| 25 | 4478 | 5164 | − | 645076413 |
| 26 | 277066 | 278082 | + | 645957989 |
| 27 | 272112 | 272231 | − | 645957989 |
| 28 | 2299803 | 2300126 | − | 646862390 |
| 29 | 1804139 | 1805368 | − | 651053129 |
| 30 | 1899111 | 1899692 | + | 646862390 |
| 31 | 209129 | 211012 | + | 637000460 |
| 32 | 487880 | 489091 | + | 640753011 |
| 33 | 1401479 | 1401856 | − | 637000098 |
| 34 | 245 | 686 | − | 647008393 |
| 35 | 2210164 | 2210319 | − | 651053129 |
| 36 | 16441 | 17124 | + | 645076159 |
| 37 | 85620 | 86303 | + | 645958072 |
| 38 | 242193 | 244169 | + | 648231718 |
| 39 | 177946 | 180372 | + | 647535981 |
| 40 | 38140 | 39216 | + | 645958123 |
| 41 | 77988 | 79490 | − | 645958383 |
| 42 | 5009 | 5929 | − | 645958068 |
| 43 | 78131 | 78853 | + | 645958189 |
| 44 | 221049 | 221729 | + | 640753011 |
| 45 | 44419 | 44616 | + | 647008364 |
| 46 | 12682 | 13458 | − | 651053131 |
| 47 | 456367 | 457155 | + | 646862390 |
| 48 | 2096829 | 2097392 | + | 646862390 |
| 49 | 14918 | 15697 | − | 647017059 |
| 50 | 54037 | 54601 | + | 651285048 |

For examples 1-12, as well as further testes microorganisms, a consolidated list of the centroids that were found is given in the following Table 25.

TABLE 25
List of centroids in Examples

| Organism | # samples | MetaRef centroids | MetaRef centroid notes |
|---|---|---|---|
| Acinetobacter baumannii | 448 | baumannii.1380.centroids | |
| Citrobacter koseri | 108 | Citrobacter.1608.centroids | NA for C. koseri, used centroids of genus |
| Enterobacter aerogenes | 298 | aerogenes.5220.centroids | |
| Enterobacter cloacae | 397 | cloacae.3734.centroids | |
| Escherichia coli | 1144 | coli.318.centroids | |
| Klebsiella oxytoca | 389 | Klebsiella.1640.centroids | NA for K. oxytoca, used centroids of genus |
| Klebsiella pneumoniae | 1179 | pneumoniae.1641.centroids | |
| Morganella morganii | 297 | NA | NA for M. morgannii or Morganella |
| Proteus | 582 | Proteus.2318.centroids | |
| Pseudomonas aeruginosa | 1042 | aeruginosa.640.centroids | |
| Salmonella | 634 | Salmonella.713.centroids | |
| Shigella | 442 | Shigella.730.centroids | |
| Serratia marcescens | 437 | Serratia.1671.centroids | NA for S. marcescens, used centroids of |
| | | | genus |
| Stenotrophomonas | 494 | Stenotrophomonas.2333.centroids | NA for S. maltophilia, used centroids of |
| maltophilia | | | genus |
| Staphylococcus aureus | 987 | aureus.754.centroids | |
| Staphylococcus aureus | 985 | aureus.754.centroids | |

# significant centroids
before/after
annotation filtering

| Organism | before | after |
|---|---|---|
| Acinetobacter baumannii | 2859 | 810 |
| Citrobacter koseri | 0 | 0 |
| Enterobacter aerogenes | 126 | 56 |
| Enterobacter cloacae | 1417 | 763 |
| Escherichia coli | 6739 | 1625 |
| Klebsiella oxytoca | 951 | 507 |
| Klebsiella pneumoniae | 2571 | 908 |
| Morganella morganii | NA | NA |
| Proteus | 3401 | 1504 |
| Pseudomonas aeruginosa | 1256 | 315 |
| Salmonella | 2333 | 670 |
| Shigella | 2046 | 788 |
| Serratia marcescens | 537 | 299 |
| Stenotrophomonas maltophilia | NA | NA |
| Staphylococcus aureus | 1333 | 367 |
| Staphylococcus aureus | 1435 | 395 |

In Table 25 herein the number of significant centroids (with the respective p-values) found by the present method before and after the above filtering process is given. This shows that, while the best 50 gene sequences are given for the Examples in the odd Tables (Tables 1, 3, 5, etc.), more than those 50 gene sequences were actually found for all the examples after filtering, e.g. 810 for Acinetobacter baumannii.

As noted above, for the even Tables (Tables 2, 4, 6, etc.) the data are further processed.

As can be seen from the Examples, similarities in gene sequences can be found over all the bacterial species, showing that they form clusters, which can also be seen in the fact that for several bacteria centroids from other bacterial species are found as suitable, e.g. from E.coli.

A genetic test for the combined pathogen identification and antimicrobial susceptibility testing direct from the patient sample can reduce the time-to actionable result significantly from several days to hours, thereby enabling targeted treatment. Furthermore, this approach will not be restricted to central labs, but point of care devices can be developed that allow for respective tests. Such technology along with the present methods and computer program products could revolutionize the care, e.g. in intense care units or for admissions to hospitals in general. Furthermore, even applications like real time outbreak monitoring can be achieved using the present methods.

Compared to approaches using MALDI-TOF MS, the present approach has the advantage that it covers almost the complete genome and thus enables us to identify the potential genomic structural changes that might be related to resistance. While MALDI-TOF MS can also be used to identify point mutations in bacterial proteins, this technology only detects a subset of proteins and of these not all are equally well covered. In addition, the identification and differentiation of certain related strains is not always feasible.

The present method allows computing a best breakpoint for the separation of isolates into resistant and susceptible groups. The inventors designed a flexible software tool that allows to consider—besides the best breakpoints—also values defined by different guidelines (e.g. European and US guidelines), preparing for an application of the GAST in different countries.

The current approach enables
a. Identification and validation of markers for genetic identification and susceptibility/resistance testing within one diagnostic test
b. Validation of known drug targets and modes of action
c. Detection of potentially novel resistance mechanisms leading to putative novel target/secondary target genes for new therapies

The current method is faster than classical culture-based predictions and has the potential to be faster than tests based on single bases, e.g. SNPs. Most remarkably the current method can help improve the accuracy of novel genetic tests.

With the present methods, structural variations can be detected, which particularly also include copy-number variations that can lead to gene dosage effects.

1. A method of determining structural variations in a genome of a microorganism, particularly a bacterial microorganism, comprising at least a change in the genome comprising more than one base, comprising:
obtaining or providing a first data set of gene sequences of a plurality of clinical isolates of the microorganism,
wherein optionally at least a part of the gene sequences of the first data set are assembled; and/or
obtaining or providing a first data set of gene sequences of a plurality of clinical isolates of the microorganism and aligning the gene sequences of the first data set to at least one, preferably one, reference sequence;
analyzing the gene sequences of the first data set for structural variations of the genome comprising at least a change in the genome comprising more than one base to obtain a third data set of structural variants;
providing a second data set of antimicrobial drug, e.g. antibiotic, resistance and/or susceptibility of the plurality of clinical isolates of the microorganism;
correlating the third data set with the second data set and statistically analyzing the correlation; and
determining the structural variations in the genome of the microorganism associated with antimicrobial drug, e.g. antibiotic, resistance.

2. The method of claim 1, wherein the structural variations are detected alignment-free.

3. The method of claim 1, wherein the structural variations are detected alignment-free and wherein the structural variations are annotated to a pan-genome of the microorganism and/or annotated to one or more reference sequences.

4. A diagnostic method of determining an infection of a patient with a microorganism, particularly a bacterial microorganism, potentially resistant to antimicrobial drug treatment, comprising the steps of:
a) obtaining or providing a sample containing or suspected of containing the microorganism, particularly the bacterial microorganism, from the patient;
b) determining the presence of at least one structural variation in at least one genetic sequence of the microorganism, particularly bacterial microorganism, as determined by the method of claim 1, wherein the presence of said at least one structural variation is indicative of an infection with an antimicrobial drug resistant microorganism in said patient.

5. A method of selecting a treatment of a patient suffering from an infection with a potentially resistant microorganism, particularly a bacterial microorganism, comprising the steps of:
a) obtaining or providing a sample containing or suspected of containing the microorganism, particularly the bacterial microorganism, from the patient;
b) determining the presence of at least one structural variation in at least one genetic sequence of the microorganism, particularly bacterial microorganism, as determined by the method of claims 1, wherein the presence of said at least one structural variation is indicative of a resistance to one or more antimicrobial drugs;
c) identifying said at least one or more antimicrobial drugs; and
d) selecting one or more antimicrobial drugs different from the ones identified in step c) and being suitable for the treatment of the infection with the microorganism, particularly the bacterial microorganism.

6. (canceled)

7. (canceled)

8. The diagnostic method of claim 4, wherein the structural variations are detected alignment-free.

9. The diagnostic method of claim 4, wherein the structural variations are detected alignment-free and wherein the structural variations are annotated to a pan-genome of the microorganism and/or annotated to one or more reference sequences.

10. The method of claim 5, wherein the structural variations are detected alignment free.

11. The method of claim 5, wherein the structural variations are detected alignment free and wherein the structural variations are annotated to a pan-genome of the microorganism and/or annotated to one or more reference sequences.
